SEQUENCE LISTING (171 sequences)

SEQ ID NO: 1 - protein, 1281 aa, synthetic construct
MKKSIFDQFVNQYALSKTLRFELKPVGETGRMLEEAKVFAKDETIKKKYEATKPFFNKLHREFVEEALNEVELAGLPEYFEIFKYWKRYKKKFEKDLQKKEKELRKSVVGFFNAQAKEWAKKYETLGVKKKDVGLLFEENVFAILKERYGNEEGSQIVDESTGKDVSIFDSWKGFTGYFIKFQETRKNFYKDDGTATALATRIIDQNLKRFCDNLLIFESIRDKIDFSEVEQTMGNSIDKVFSVIFYSSCLLQEGIDFYNCVLGGETLPNGEKRQGINELINLYRQKTSEKVPFLKLLDKQILSEKEKFMDEIENDEALLDTLKIFRKSAEEKTTLLKNIFGDFVMNQGKYDLAQIYISRESLNTISRKWTSETDIFEDSLYEVLKKSKIVSASVKKKDGGYAFPEFIALIYVKSALEQIPTEKFWKERYYKNIGDVLNKGFLNGKEGVWLQFLLIFDFEFNSLFEREIIDENGDKKVAGYNLFAKGFDDLLNNFKYDQKAKVVIKDFADEVLHIYQMGKYFAIEKKRSWLADYDIDSFYTDPEKGYLKFYENAYEEIIQVYNKLRNYLTKKPYSEDKWKLNFENPTLADGWDKNKEADNSTVILKKDGRYYLGLMARGRNKLFDDRNLPKILEGVENGKYEKVVYKYFPDQAKMFPKVCFSTKGLEFFQPSEEVITIYKNSEFKKGYTFNVRSMQRLIDFYKDCLVRYEGWQCYDFRNLRKTEDYRKNIEEFFSDVAMDGYKISFQDVSESYIKEKNQNGDLYLFEIKNKDWNEGANGKKNLHTIYFESLFSADNIAMNFPVKLNGQAEIFYRPRTEGLEKERIITKKGNVLEKGDKAFHKRRYTENKVFFHVPITLNRTKKNPFQFNAKINDFLAKNSDINVIGVDRGEKQLAYFSVISQRGKILDRGSLNVINGVNYAEKLEEKARGREQARKDWQQIEGIKDLKKGYISQVVRKLADLAIQYNAIIVFEDLNMRFKQIRGGIEKSVYQQLEKALIDKLTFLVEKEEKDVEKAGHLLKAYQLAAPFETFQKMGKQTGIVFYTQAAYTSRIDPVTGWRPHLYLKYSSAEKAKADLLKFKKIKFVDGRFEFTYDIKSFREQKEHPKATVWTVCSCVERFRWNRYLNSNKGGYDHYSDVTKFLVELFQEYGIDFERGDIVGQIEVLETKGNEKFFKNFVFFFNLICQIRNTNASELAKKDGKDDFILSPVEPFFDSRNSEKFGEDLPKNGDDNGAFNIARKGLVIMDKITKFADENGGCEKMKWGDLYVSNVEWDNFVANK

SEQ ID NO: 2 - protein, 1332 aa, synthetic construct
MKSIFDNFTGLYSLSKTLRFELRPVGQTLENIKNGHFLESDKKMADDYQDVKKIIDNYHKFFIDDVLKGASFDWALLEKELTDFNKNKTDDSKVEAEQKKLREQIAKTLAGDKRFKSLTASTPNDLFNKDKDFIGWLEQSSVKEIRKDALDTFKKFSSYFKGFQENRKNVYSADDIPTAVPYRIVNDNFPKFLQNISIFKTIQEKCPQVIADVENELASYLGKEKLADIFTVQAFNKYLCQGGKENQRGIDFYNQVIGGIAEKEGGVNLRGINQFLNLYWQQHPDFAKENRRIKMVPLYKQILSDRSSLSFKIETIDTDEELKTAISEYADKLESKSNDEKKSVLDVCVELFDSIKEQNLQEIYVNRKDINNISRILTGDWSWLQSRMNLYADEVFTTKAEKTRWQKSVDGDEGENKSKGAYSLAELNRVLEYASENVAETDIRITDYFCHRNRFYYEKESGLFKQGEELVALSIKESCEDILSKRKAMNEAFANISESNSLRDNSEDIAKTKTYLDSVQDLLHRIKPLKVNGLGDPSFYAVFDSIYSALSEVISIYNKTRNYITRKAESPEKYKLNFDNPTLANGWDLNKEKDNTCVLLRKNGMYYLGIMNPKDKPKFAEKYDCGTESCYEKMIYKLLPGPNKMLPKVFFSTKGKKQYNPPENILHGYEQGKHKKGVAFDINFCHELIDWFKSAINQHEDWKKFGFKFSDTKSYKDISDFYREVTEQGYKLTFINIPESEISKMVSEGKLYLFQIYNMDFAPGANGMPNMHTLYWKNLFSEENLKDVVLKLNGEAELFYRPAGIKEPVVHAKGSYLVNRITKDGEPIPEKIHDEIYRNANGNLNNLSKEASEYKESHKVVIKQASHEIIKDRHYTEPKFLFHIPLTINFKAPSMKPTVCTAIQNENVRRFLKNNPDVNIIGLDRGERNLIYLSLINQKGEIIKQFSFNDVEREQNGQTVKVNYHEKLDQREKERDAARKSWQAIGKIAELKEGYLSAVIHQLTKLMVEYNAIVVMEDLNFGFKRGRFHVEKQVYQKFEHMLIDKLNYLVFKDRGLNEPGGALNGYQLAGQFESFQKLGKQSGMLFYVPAAYTSKIDPKTGFVSMMNFKDLTNVHKKRDFFSKFEDIHFDEATCSFVFTFDYKNFNGKAKEEMKQTKWAVYSREKRIVYFSKTKSSEDIMPTEKLRALFESDGIEYKSGNNIHDSVMAVGADLKEGAKPSKEIADFWDGLLYNFKLILQMRNSNAKTGEDYIISPVMASDGTFFDSRVEAKKGKDAKLPLDADANGAYHIALKGLSLINKINLAGEDELKKFDMKISNEDWFNFAQEKKYAE

SEQ ID NO: 3 - protein, 1345 aa, synthetic construct
MTKQNKSVTQNTKRKNFGEFTNLYSLSKTLRFELKPVKATKTILEVERKDRENFKKDRKIAKNYQKLKGILNELHQEFIQDVMREFSFQEKEIKEFEERYLEALNFKEKDNYKKRTQLKNAYEKVAKKLAGKIATAFGKYNQEKYGVKFTKKNLTGENVFDILEGKYKGDKKILGIIHTFKFKPTKEEKKQGKEAVNFSTYLGGFNQNRENFYKGEMKAGQFATRTIENLIQFLKNKKLFIDKYKDNYQKIGFSQEQVEIFNLNYFNNLFLQEGLDVYNGILGAKKGEKNTENDGLNQKINLFKQKEKTRCKANGEKFNKSDYPIFKELYKQIGSIKKDNDVYVEIKSDEELVNVLQSLPEKTANTLREVQKFYENFFDKIFNDEFDLDKIYLPKSVGTHFSHLAFSDWSKLAFVFNKRWRNEKVKIKEGEDVNVQSRSLADIKKRMEEILEMDGGVSFGKTYCQKVGLEKEARTIEDVWSGFWKIIQYHINSQFIGGEKEVFDKEKKDDKTEKIQTIDDLQEEYLQATEMYRERMVESEEGLNDGEEKEIKTKLKNYLDRIKDIERIARYFDLRKHFDDIDEASKDGDFYFIYQELLQDISEAKINDHYNEIRNYLTKANVVDDKFKLNFNDGQTLSGWDLNKETEKFSLIFKRKVDGGVEYYLGIINKEKNKTIFDKKKHPEIFTENSEFEKMEYKLFPSPSKMLPKIAFTKNKEGERIKPVFLDENAGKEIAQIKKEFALFQDAKKEDKNKWSDEFDRKKLNKLIDYYKLVLEKHPEKYMQTFNFVFKSSAKYKNLGEFNDDVARQNYVTKFVSVDKDYIDQKVESGELYLFKIHNKDWNLTKAGDTKKQSKKNLHTIYFEELFSEKNIAEPVFKLSGGAEVFFRDAIEKKKQKKKKDKKGKEILEKFRFTKNKILFHVPITINYGKPSINQGQFNQKINEFIADNSRSVNILGIDRGEKHLLYYSLVDNNGKIIKSGSLNEINGVDYHEKLDKAEKERQEARKSWQKINQIKNLKAGYISQVIKKIVDLAIENNAIIVLEDLNFGFKSFRQKIEKNVYQQFEKALIDKLGFVTDKEKLNHRQAPQLSAPFESFEKMGKQTGIVFYVLATNTSKVCPQCQWKKNIFPHYSTKKSIAENLQKQYKMKMYWRENENRFEFEYKGDGDKEFSSIFSNVDRVRYDKRANNNQGGYVIYQIDSTTKEKDGRNIKEKSITNLLKELLLEKFEIDNLEGELLVKLSEKSPDVSKETIKDFFGLLNSILNIRNSMTDTEEDYIQCPACGFDTRKENKIGIKNGDDNGAYNIALRGRFLIERIKKAKKEDKKPNLTFSNNDYFQWVREFVK

SEQ ID NO: 4 - protein, 1280 aa, synthetic construct
MFNNFIKKYSLQKTLRFELKPVGETADYIEDFKSEYLKDTVLKDEQRAKDYQEIKTLIDDYHREYIEECLREPVDKKTGEILDFTQDLEDAFSYYQKLKENPTENRVGWEKEQESLRKKLVTSFVGNDGLFKKEFITRDLPEWLQKKGLWGEYKDTVENFKKFTTYFSGFHENRKNMYTAEAQSTAIANRLMNDNLPKFFNNYLAYQTIKEKHPDLVFRLDDALLQAAGVEHLDEAFQPRYFSRLFAQSGITAFNELIGGRTTENGEKIQGLNEQINLYRQQNPEKAKGFPRFMPLFKQILSDRETHSFLPDAFENDKELLQALRDYVDAATSEEGMISQLNKAMNQFVTADLKRVYIKSAALTSLSQELFHFFGVISDAIAWYAEKRLSPKKAQESFLKQEVYAIEELNQAVVGYIDQLEDQSELQQLLVDLPDPQKPVSSFILTHWQKSQEPLQAVIAKVEPLFELEELSKNKRAPKHDKDQGGEGFQQVDAIKNMLDAFMEVSHAIKPLYLVKGRKAIDMPDVDTGFYADFAEAYSAYEQVTVSLYNKTRNHLSKKPFSKDKIKINFDAPTLLNGWDLNKESDNKSIILRKDGNFYLAIMHPKHTKVFDCYSASEAAGKCYEKMNYKLLSGANKMLPKVFFSKKGIETFSPPQEILDLYKNNEHKKGATFKLESCHKLIDFFKRNIPKYKVHPTDNFGWDVFGFHFSPTSSYGDLSGFYREVEAQGYKLWFSDVSEAYINKCVEEGKLFLFQIYNKDFSPNSTGKPNLHTLYWKGLFEPENLKDVVLKLNGEAEIFYRKHSIKHEDKTIHRAKDPIANKNADNPKKQSVFDYDIIKDKRYTQDKFFFHVPISLNFKSQGVVRFNDKINGLLAAQDDVHVIGIDRGERHLLYYTVVNGKGEVVEQGSLNQVATDQGYVVDYQQKLHAKEKERDQARKNWSTIENIKELKAGYLSQVVHKLAQLIVKHNAIVCLEDLNFGFKRGRFKVEKQVYQKFEKALIDKLNYLVFKERGATQAGGYLNAYQLAAPFESFEKLGKQTGILYYVRSDYTSKIDPATGFVDFLKPKYESMAKSKVFFESFERIQWNQAKGYFEFEFDYKKMCPSRKFGDYRTRWVVCTFGDTRYQNRRNKSSGQWETETIDVTAQLKALFAAYGITYNQEDNIKDAIAAVKYTKFYKQLYWLLRLTLSLRHSVTGTDEDFILSPVADENGVFFDSRKATDKQPKDADANGAYHIALKGLWNLQQIRQHDWNVEKPKKLNLAMKNEEWFGFAQKKKFRA

SEQ ID NO: 5 - protein, 1281 aa, synthetic construct
MIKSYDDFTKLYSLQKTLRFELKPIGKTLEHIKKSEIIESDETLEEQYAIVKNIIDRFHRKHIDEALSLVDFTKHLDTFKTIQELYLKRGKTDREKKELEELSADLRKLIVSYLKGNVKQKTQHNLNPIKERFEILFGKELFTNEEFFTLAENKEEKKAIQAFKGFTTYFKGFQENRKNMYSEEDKSTAIAYRIINENMPLFIENIARFQKVLDVIEKTKLTELKQNLKTELKGHSVSDIFRIEYFNNVLTQEGISRYNTIIGGKTTTEGVKIQGLNEIINLHNQQSKDVKLPILKPLHKQILSEEYSTSFKIKAFENDNEVLKAIDTFWNEHIEKSIHPVTGKRFNILLKIENLCKKLEKYKDKEIEKLFIERKNLSTVSHQVYGQWNIIRDALRMHLEMNNKNIKEKDIDKYLDNDAFAWKEIKDSIKIYKEHVEDAKELDENGIVKYFSSMSINEEDDEKEYSISLIKNINEKYNNVKSILEEDRTGKSDLHQDKEKVAIIKEFLDSLKQLQWFLKLLYVTVPLDEKDYEFYNELEVYYEALLPLNSLYNKVRNYMTRKPYSVEKFKLNFYSPTLLDGWDKNKETANLSIILKKNGKYYLGIMNKENNTIFENFPKSKSNDYYEKMIYKLLPGPNKMLPKVFFSKKGLEYYKPSKEILRIYEKGEFKKDKSGNFKKESLHTLIDFYKEAIAKNEDWKIFKFKFKNTREYEDISQFYRDVEEQGYLIIFEKVDANYVDKLVEEGELYLFQIYNKDFSENKKSKGNPNLHTIYWESLFDNQNLKDVVYKLNGEAEVFYRKKSIDYPEEIYNNGHHKEELNGKFNYPIIKDRRYTQDKFLFHVPITMNFISKEEKRVNQLACEYLSTTKEDVHIIGIDRGERHLLYLSLIDKEGNIKKQLSLNTIKNENYDKEIDYRVKLDEKEKKRDEARKNWDVIENIKELKEGYMSQVIHIIAKMMAEEKAILIMEDLNIGFKRGRFKVEKQVYQKFEKMMIDKLNYLVFKNKEPLEPGGSLNAYQLTSKFDSFKKLGKQSGFIFYVPSAYTSKIDPTTGFYNFIRVDVPNLEKGKEFFSKFEKIIYNTKEDYFEFHCKYGKFVPEPKNKDNDRKTKESLTYYNAIKDTVWVVCSTHHERYKIVRNKAGYYESQPVDVTKNLRDIFSEANINYSDGKDIKPIIIESNNAKLLKSIAEQLKLILAMRYNNGKHDDDEKDYILSPVKNKQGKFFCSLDGDQSLPINADANGAYNIALKGLLLIEKIKKQQGKAKDLYISNLEWFMFMMSR

SEQ ID NO: 6 - protein, 1265 aa, synthetic construct
MRTTTSLDAFTNRYALSKTLRFELKPIGNTQMMLEQNNVFAKDRAIREKYEKTKPWIDLLHREFVAESLQNAQLGNLDDYYAALQNVQKITKDTNAEDKKRWKKGFEKQEKRLRKEVVALFDKAAHIWATQRYPQLKKKTKDFLFEEGVFEHVLFARYGSAPDTTVKIVTSNPETGEVIDEREESIFKGWKGFTGYFDKFFETRKNFYKDNGTATAIATRAINQNLRRFAENMQKLTDIKNNYPELLAHTDFGDFDIAHAQSLDFYARTCLLQEGIDAYNKKFVGVLKSAINEYQQKNKGVRISYPKTLDNQILGERERRLFDVIEDDRELHDVFRAFVDDGTVFAAEMRQLAQAFSAQNGTYDYTQIYISKKGFETISRKYTHDTRAWHDALADVFKAKAKKRIATTASGEKKFPAYIPVAYITQALTLVQESEDTECTWKERYASITENKTLEEGFFAIFADEFERLFVHMEATVQDTDYVVAEDKAKKLLSDGQITKNEQTTQIIKEYADALLRIYQMAKYFAVEKKSMWDDAVAIDDTFYETFKEIYGNTHSTIVASYNLLRNYLTKKPWEDVQKWKLNFENPTLLDGWDKNKEAANFGVILRDGDKFYLGIMRKGHNNIFANQHHSNFEGQGLQKMVYKFFPDPKKMFPKVCFSAKGMEFFAPSEEIVRIYKNAEFKSGDTFNVESMQKLIDFYKNALQKYDGWKIYDFKHLKDTAQYTSNIGEFYDDVAKGGYQLGWQNISKEYVEEKNANGELYLFQIKNKDWNDGATGRKNLHTLYFEYLFSEKNAAADFVFRLNGGAEVFYRPAAIESKTERRGNREVAAKKRYTQDKVFLHVPITLNRTAGDVKTSAFNDAVNRFLAGNPDINIMGIDRGEKHLAYYSIIDQNGNRIVSGSFNTIGSKDYHALLTERQGAREEARKNWQRVEQIKDLKKGYISLVVREIADLAIKHNAIIVLENLNMRFKQIRGGIEKSVYQQLEKALIEKLNFLVNKGEVDATKAGHLLRAYQLAAPFETFEKMGNQTGIIFYTTASYTSQVDPVTGWRPHVYLKYRNAQKTKEDILRIFDDIVFNDEKQRFEFAYRHNGVSWTVCSSVERHRWNRSNNAGKGGYDVFPVEGEGSITQRLQEACAQRGIDTTRNILAQIDELDESASATVSFLRDLCFYFRLICQIRNTDDGADDINAQDFLMSPVEPFFDTRNAQEQYPQNGDENGAYNIARKGIIILQKITAWGRSQDTQRRYPDTFVSQDEWDTFLTQHTT

SEQ ID NO: 7 - protein, 1281 aa, synthetic construct; identical to SEQ ID NO: 5

SEQ ID NO: 8 - protein, 1257 aa, synthetic construct
MKYTDFTGIYSVSKTLRFELIPQGSTVENMKREGILNNDMHRADSYKEMKKLIDEYHKAFIERCLSDFSLKYDDTGKHDSLEEYFFYYEQKRNDKTKKIFEDIQVALRKQISKRFTGDTAFKRLFKKELIKEDLPSFVKNDPVKTELIKEFSDFTTYFQEFHKNRKNMYTSDAKSTAIAYRIINENLPKFIDNINAFDIVAKVPEMQEHFKTIADELRSHLQVGNDIDKMFNLQFFNKVLTQSQLDVYNAVIGGKSEGNKKIQGINEYINLYNQQHKKARLPMLKLLYKQILSDRVAISWLQDEFDNDQDMLDTIEAFYNKLNSNETGVLGEGKLKQILMGLDGYNLDGVFLRNDLQLSEVSQRLCGGWNIIKDAMISDLKRSVQKKKKETDADFEERVSKLFSAQNSFSIAYINQCLGQAGIRCKIQDYFACLGAKEGENEAETTPDIFDQIAEAYHGAAPILNARPSSHNLAQDIEKVKAIKALLDALKRLQRFVKPLLGRGDEGDKDNFFYGDFMPIWEVLDQLTPLYNKVRNRMTRKPYSQEKIKLNFENSTLLNGWDLNKEHDNTSVILRREGLYYLGIMNKNYNKIFDANNVETIGDCYEKMIYKLLPGPNKMLPKVFFSKSREQEFSPSKKILEIWESKSFKKGDNFNLDDCHALIDFYKDSIAKHPDWNKFNFKFSDTQSYTNISDFYRDVSQQGYSLSFTKVSVDYVNRMVDEGKLYLFQIYNKDFSPQSKGTPNMHTLYWRMLFDERNLHNVIYKLNGEAEVFYRKASLRCDRPTHPAHQPITCKNENDSKRVCVFDYDIIKNRRYTVDKFMFHVPITINYKCTGSDNINQQVCDYLRSAGDDTHIIGIDRGERNLLYLVIIDQHGTIKEQFSLNEIVNEYKGNTYCTNYHTLLEEKEAGNKKARQDWQTIESIKELKEGYLSQVIHKISMLMQRYHAIVVLEDLNGSFMRSRQKVEKQVYQKFEHMLINKLNYLVNKQYDATEPGGLLHALQLTSRMDSFKKLGKQSGFLFYIPAWNTSKIDPVTGFVNLFDTRYCNEAKAKEFFEKFDDISYNDERDWFEFSFDYRHFTNKPTGTRTQWTLCTQGTRVRTFRNPEKSNHWDNEEFDLTQAFKDLFNKYGIDIASGLKARIVNGQLTKETSAVKDFYESLLKLLKLTLQMRNSVTGTDIDYLVSPVADKDGIFFDSRTCGSLLPANADANGAFNIARKGLMLLRQIQQSSIDAEKIQLAPIKNEDWLEFAQEKPYL

SEQ ID NO: 9 - protein, 1259 aa, synthetic construct
MLKEKQFKTFGDFTNLYELSKTLRFELRPTPETKDLLDKNKIIQTDKKIAENYQEIKKYFDKLHKKFIKEALSNTQIDFSDFCKLWEQNSKDSGKIKDLSRKLRKSIKQAFDKKGADWHKRYLEKGIKLKKKNLDILFEERVLDILKEEFKDDVDVKLFESFKGFSTYFTNFHESRKNFYKDDGTASAIATRIIDENLKRFCDNIKVKKHSKKLISELNEREAKIFEADFYNRCLLQQGIDDYNQVIGDINKKINNLRQNKIENTPTLKILYKQILGDVRRQETEQDAFIEIKNNEEVFDFLQDFIKHSDENNKYFKNLFYKFIEGKHSLDKIFLAKRFVNTISGKWFASWEVFGAELIKKFGNKKDLPDFIPFAAVKDVLQNCNIPANELFKEKIKNDEDKNIYDIFINLWKEEFDSNLKKVEESKKEVENMIAEDKVYSNKKEKRKNDNGEEIEIEIQKEKIKNYADAAMNIFRMMKYFLLEKNGKTVEGMGEDNNFYNELNIVFKGGEIDGKVYEGVKTYLYYNEFRNYLTKKPFNEEKTKLNFDCGQILSGWDKNKESEKLGVILRKDNKYYLAIINKKHNKIFDVKKNSYAYIVGDNFYEKMEYKLFPDAKRMIPKIAFAKNNKEKFGWTDEIQKIKNEYAEFQEGKKNDKNLWKDKFNKNKMEKLITYYQNCLEKGGYKDIYNFRWKSPDKYSGIGEFNDEIDRQSYCLKFVKVDFNYVFEKVKSGELYLFQIYNKDFSDKADRAQKENIHTEYFKLLFDQRNLDNVVLKLSGGAEIFYRPKTEGLPKKKDNKGNEVVRHRRYADDKYFLHLPIQLNFGRGNLSGGEFNSKINQYLSEQREIKIIGIDRGEKHLAYYSVINQDGKIEEIESLNTVNGIDYRKKLDELEKKREQERKSWQSISKIKDLKKGYISHVIKKICDLAIEHNAIIVFEDLSGGFKNSRKKIEKQIYQNLELALATKLNYLTFKDKNFGESGHYLNAYQLAPKIDNYQDIKMQTGIVFYTPAGYTSSTCPQCGFRKTLKFDYTATISKAEDLIRGSKLNIVFEKEKNRFKINYLFNPIEKKKKKIKENELFADAGAKNEFTIYSDVKRIKWHNTGTKRLEEAEGERLLENKNSRGRDKEYDINKCLTRLFRENKIDVNGDIIGQITKIKSLKLYQDLFYYLFLATLIRNNVSGSDIDYIQCPSCHFHSDGGFQKQKFNGDANGAYNIARKGILILKKIKQFAAQDKDMKNFGWKHLTVDINEWDKFTQK

SEQ ID NO: 10 - protein, 1371 aa, synthetic construct
MNKNFSNFTELYTLSKTLRFELKPVAQTKENIKKGKFLESDKKKADDYKDVKKIIDNYHKFFIDDVLKNASFDWTVLEKEMSDFNKSKADDSKVEAEQKKLRDQIAKKLTSDKRFKALTASTPSDLFNKDKDFIDWFTQNSTKDINKEALETFKRFSSYFKGFQENRKNVYSAEPIPTAVPYRLVNDNFPKFLQNIALFKIIQEKCPQVISDVEKELASYLGKEKLADIFTVQAFNKYLCQGGKENQRGIDFYNNILGGIAEKEGGINLRGINQFLNLYWQQHQDFAKQNKRIKMIPLFKQILSDRSSLSFKIESINTDQELLTSITEYADKLETKSNDEKKSVLDICSDLFASIKAQNLQEIYVNRKDINSISRILTGDWSWLQSRMNVYADEVFTTKAEKTRWQKSIDGDEGENKSKGVFSLAELNSVLEYSSENVSETDVRITDFFDHRNRFYYEKESGLFKQGDELVALSIRESCEDILAKRKAMDEAFANVSENNSLRDNSEDVAKIKIYLDCVQELLHRIKPLKVNGLGDPAFYAVFDTVYNSLSEVISLYNKTRNYITKKAANPEKYKLNFDNPTLADGWDLNKEQANTSVLMRKDGMYYLGIMNPKDKPKFAEKYECGNEACYEKMIYKQFDATKQIPKCSTQVKEVKKHFQSGATDSIILNDKSKFKLDLVITKEIWFLNNHVWNGEKFVPKRESNETRPKKFQIGYYKQTGDLGGYKEALNIWISFCKTFLQSYISSSIYDYDFKESSNYDSLDEFYNYLNATCYKLSFINIPEATISQMVSEGKLYLFQIYNKDFAPGASGMPNMHTLYWKNLFSEENLKNVVLKLNGEAELFYRPAGIKEPVIHAKGSYLVNRITKDGEPIPEKIHDEIYRNANGKLESLSKEATEYKASHKVVIKEAKHDIIKDRHYTEPKFLFHVPLTINFKASGNSYINENVRRFLKNNPDVNVIGLDRGERHLIYLSLINQKGEIIKQFTFNEVERNKNGQVIKVNYHEKLDQREKVRGAARKSWQAIGKIAELKEGYLSAVIHQLTKLMVEYNAIVVMEDLNFGFKRGRFHVEKQVYQKFEHMLIDKLNYLVFKDRGLTEAGGVLNGYQLAGQFESFQKLGKQSGMLFYVPAGYTSKIDPKTGFASMSNFKDLTNVHKKRAFFSKFDDIHFDDATGSFVFTFDYKNFDGKAKEEMKRTKWSVYSKDKRIVYLSKTKSYEDVQPTEKIKASLESVGIEYMSGNNLIDSIMVIGAELKDGAKPSKEIADFWDRLLYNFKLILQMRNSNAKTGEDYIISPVMADDGTFFDSREEFKKGENAKMPVDADANGAYHIALKGLSLLKRFDAASENELKKFDMKISNVDWFKFAQEKSYAE

SEQ ID NO: 11 - protein, 1261 aa, synthetic construct
MNFDEFTNLYSISKTLRFELKPVGKTLDWITKNKLVEEDEKRAEDYKTVKKIIDRYHKEFIEEAFESALNANSFQRDMKDLIESYSELYYKEDKSDKDKKELENISSQMRQEIVSCFEGNYSDEVKKKFSNLFKKELIKQDLLNFCQPSEKEVIEKFADFTTYFGGFHENRKNMYSSEEKSTAISHRIVHENLPRFLDNLKVFNSLKEEYQDFPWNQLEFNLKNIDPSLKLEDYFSKKGFILTFTQKGIDRYNLILGGKSLDSGQKVQGLNELINLYRQKKCLDRRKIPNLKELYKQILSDRVKFSFIPEKFLSDNSLLEAIRNFHEEVINAKIEIDSKQVNFLEALKNTIQGIKAHKLEKIYISNDLGLTNISNFLYGEWSKIKSALFYYYDEKIADKGKGDKQSKKYQEGREKWIKKDYYSVQKLNEAITLYSKYSEDGFEATPLEEYFSKFKTRDENRAELDILSNIEQSYSELKPLLEKEYLKNKNLKSDKYNIEKIKNYMDSIKILQNFLKPLTPKKILEEKDLGFYNEFETYVESLQSFNRLYNKVRNYLTEKEYSVEKFKLNFENSTLLNGWDENKESANLGIILRDEDKYYLGIMNKNYKKIFSNIPKLKSSEAYYEKMVYKLLPGANKMLPKVFFAKSNIGFFQPSQKILDIKNSESFKKGDNFNLKDCHELIDFYKESISKHEDWSKFNFHFSETSSYQDISEFFREVEAQGYKITFNQVSKDYIHSLVEYGKLYLFQIYSKDFSEKKKSNGKPNLHTIYFKALFDEENLKDVVVKLNGEAEVFYRKKSINYDPEIREKGHHYEELKDKFTYPIIKDKRYAEDKFHFHFPITLNFKPKERSNFNLEVRKFLKASKDVNIIGIDRGERHLLYLVMINQEGKILKQLSLNTIQNEKQAVNYKEILQKREIARNQARKSWRTVENIKELKEGYLSQVVHKISKMMVEHKAIVVLEDLNLGFKRGRQKVERQVYQKFEKMLIDKLNFLVLKDRKNNEPGGILKAYQLTDKFVSFENIGKQTGFLFYVPAWNTSKIDPKTGFVNFLHLKYENVQQAKDLIGKFDSIRYNKQKDWFEFQVTTDKFFTKENLPESRTWTICSTPTTRYYTEKTTNSSIVTNEINVNEKLKELLSEFHFEDGKELKEKILQKDTKDFFSKLIFYLRVLTSLRQNNGKIGDQEKDFILSPVADSKGNFFNSLEAKDDEPNDADANGAYHIALKGLMNLQVLKQTQEENLDKPNWKIKNTDWLHFVWNRQN

SEQ ID NO: 12 - protein, 1261 aa, synthetic construct
MNFDKFTNLYSISKTLRFELKPVGKTLDWITKNKLVEEDEKRAEDYKTVKKIIDRYHKEFIEEAFESALNANSFQRDMKDLIESYSELYYKEDKSDKDKKELENISSQMRQEIVSCFEGNYSDEVKKKFSNLFKKELIKQDLLNFCQPSEKEVIEKFADFTTYFGGFHENRKNMYSSEEKSTAISHRIVHENLPRFLDNLKVFNSLKEEYQDFPWNQLEFNLKNIDPSLKLEDYFSKKGFILTFTQKGIDRYNLILGGKSLDSGQKVQGLNELINLYRQKKCLDRRKIPNLKELYKQILSDRVKFSFIPEKFLSDNSLLEAIRNFHEEVINAKIEIDSKQVNFLEALKNTIQGIKAHKLEKIYISNDLGLTNISNFLYGEWSKIKSALFYYYDEKIADKGKGDKQSKKYQEGREKWIKKDYYSVQKLNEAITLYSKYSEDGFEATPLEEYFSKFKTRDENRAELDILSNIEQSYSELKPLLEKEYLKNKNLKSDEYNIEKIKNYMDSIKILQNFLKPLTPKKILEEKDLGFYNEFETYVESLQSFNGLYNKVRNYLTEKEYSVEKFKLNFENSTLLNGWDENKESANLGIILRDEDKYYLGIMNKNYKKIFSNIPKLKSSEAYYEKMVYKLLPGANKMLPKVFFAKSNIDFFQPSQKILDIKNSESFKKGDNFNLKDCHELIDFYKESISKHEDWSKFNFHFSETSSYQDISEFFREVEAQGYKITFNQVSKDYIHSLVEYGKLYLFQIYSKDFSEKKKSNGKPNLHTIYFKALFDEENLKDVVVKLNGEAEVFYRKKSINYDPEIREKGHHYEKLKDKFTYPIIKDKRYAEDKFHFHFPITLNFKPKERSNFNLEVRKFLKASKDVNIIGIDRGERHLLYLVMINQEGKILKQLSLNTIQNEKQAVNYKEILQKREIARNQARKSWGTVENIKELKEGYLSQVVHKISKMMVEHKAIVVLEDLNLGFKRGRQKVERQVYQKFEKMLIDKLNFLVLKDRKNNEPGGILKAYQLTDKFVSFENIGKQTGFLFYVPAWNTSKIDPKTGFVNFLHLKYENVQQAKDLIGKFDSIRYNKQKDWFEFQVTTDKFFTKENLPESRTWTICSTPTTRYYTEKTTNSSIVTNEINVNEKLKELLSEFHFEDGKELKEKILQKDTKDFFSKLIFYLRVLTSLRQNNGKIGDQEKDFILSPVADSKGNFFNSLEAKDDEPNDADANGAYHIALKGLMNLQVLKQTQEENLDKPNWKIKNTDWLHFVWNRQN

SEQ ID NO: 13 - protein, 1330 aa, synthetic construct
MNRTLDDFTNLYSLSKTLRFELKPIAQTEENIRKGKFLESDKRKSDDYKDVKTIIDNYHKYFIDDVLKNACLDWSQLEKEIIEYNKNKSDDSALAAEQKKLREEINKLFSTDKRFKALTAATPNDLFKSILPEWFGNNELPGVNKQAVETFQKFSSYFTGFQENRQNVYKSEPIPTAVPYRLVNDNFPKFLQNIVVFKIIQEKCPQVISDVENELASYLGNEKLKDIFAVSSFNKYLAQKGAEYQKGIEFYNQILGGIVEQESGQNLRGINQFLNLYWQQHPDFAKNNRRIKMVPLFKQILSDRSSLSFKIASIESDEELKAALLECADRLEESTDDKNSVFEECLDLFGTIKDQNLNEIYINRKDINSVSRILTGDWSWLQSRMNVYADEKFTTKAEKARWQKTIDGDEGENKSKGFYSLAELNNVLEYSSENVSETEIRITDYFEHRNRFYIEKETDNFIQGDEFVAFSIKESCSDILSKRKNMNSALENHSQEHPLRDDSGTVEIIKAYLDSVQDLLHRIKPLKVNGVGDASFYSAFDDIYASLSEVVSVYNKTRNYITKKAASPEKYKLNFDNPTLADGWDLNKEQANTSILLRKDGMYYLGIMNPKNKPKFLEKYENTNNEKCYEKMIYKLLPGPNKMLPKVFFSTKGKEVFNPPEYILQGYEQGKHKKGESFDISFCHELIDWFKSAINQHEDWKKFDFHFSDTKTYRDISDFYREVTEQGYKLTFINIPESEIQAKVSEGKLYLFQIYNKDFAKGATGMPNMHTLYWKNLFSEENLKNVVLKLNGEAELFYRPAGINEPIVHKAGSYLVNKVSEDGETIPDSIYQEIYKNANGMLNSLSEVAERYKAEHNVIIKKAAHDIIKDKHYTEAKFLFHVPLTTNFMAPSMKPAVCTAIQNENVRKFLKNNPDVNIIGLDRGERHLIYLSLINQKGEIIKQFTFNEVERKKNNQIVKVNYHDKLSQREKERDAARKSWQTIGKIAELKEGYLSAVIHQLTKLMVEYNAIVVMEDLNFGFKRGRFHVEKQVYQKFEHMLIDKLNYLVFKDRGLTEAGGVLNGYQLAGQFESFQKLGKQSGMLFYVPAGYTSKIDPKTGFASMLNFKDLTNVHKKRAFFSKFEDIHFDSVTNSFMFTFDYKNFDGKAKEEMQQTKWSVYSKDKRIVYFPKSKSYENVDVTSKLKALFDNAGIKYQSGNNILDDVMAIGADLKDGTKPSKEVAEFWDGLLYNFKLVPQMRNSNARTGEDYIISPVMADDGTFFDSREELKKCGNAKLPVDADANGAYHIALKGLSLLKRFDAADEDDLKNLKINISNSDWFKFAQEKNYAK

SEQ ID NO: 14 - protein, 1214 aa, synthetic construct
MKKFINLYPVQKTLRFELRPVGETLKNIEEKGILTADEHRSYAYGKVKKFIDEYHKAYISKSLSGLVLDASALNDVLLFSGRTDETLRNKASKAMDRLRKQIADSFDTKSLFSDKVIKEDLPAFLDDDDKTQLVAEFGNFTTYFSGFNENRKNMYTSEPKATGIAYRLINENLPKFISNMKSFGSIREPLADQIKELKSYFEPMLEGIQVEDFFSDITYYSTLLTQEMITIYNTLIGGKSEGATKTKGLNEYVNLYNQTHKDKLPKFVPLYKQILSDRDSLSWLPETFKSDGELLESVETFHRLFESRIVNGNDGTISLKELLVSMEGYDLNGIYLSTGAGLNSIMKSHTNDWDALNKAIEEDFNRKYPCTKDKDMPKFLEKRARYVKSFDSLSIADINGYIGEGKALQHYFMTLGASSDNERTLFERINESYSAIRELLNTEYNKNLIQQDEDIEKLKTYLDSIKELQWFIKPLNGTGTESGRDGRFYGDFDKLYGILDKVTPLYNMVRNYVTRKPYSEEKIKLNFDNSTLLAGWDTNKERDNSGIILRKGGRYYLAIMDKMFNKSFDLDSLPCKGECYEKMDYKLLPGPNKMLPKVFFSESRIDEFAPSKSLLEHYSKGTHKKGVDFNLNDCHELIDFFKASIEKHKDWKQFNFHFSDTVTYSDISGFYKEVEQQGYKITFRDVSCSYIDQLVNEGKLYLFQIYNKDFSEYSKGTPNMHTLYWKALFSEENLKDVVYKLNGDAEVFFRRKSIQYDSEKMKKGFHHETLKDKFSYPIIKDRRFTMDKFQLHVPITMNFKSFGINNINMMVREYIRDGEIEHILGIDRGERNLLYISLIDLNGNIVKQLSLNEIINKYNNYEYKTDYHSLLEKKEVNRLDARKNWKTIENIKEIKEGYLSQVIHIISQMIVEYKAIVVLEDLNSGFLRGRQKVEKSVYQQFEHMLINKLNYLVDKKSPNLEDGGLMRAYQFTSKFESFQKLGRQSGFLFYIPAWNTSKIDPVTGFTNFISTRYESIDSARELLGKFEDIRYNAENDWFEFVIGDYSSFNPKAVGRKEWTLCTYGTRIRSFKNEKKNNEWDCEEIHLTEEFKKLFGSHGIDYADNLMGSILIQSNKDFFSGILSLLGLMLQLRNSRTGTEEDYIISPVAGQDGRFFDSREYSSADAPLPTDADANGAYNIARKGLWAIKQISIADDVSKVNLAISNESWLKFVQG

SEQ ID NO: 15 - protein, 1321 aa, synthetic construct
MFDQFTNLYELSKTLRFELKPVGKTRENMNEHLKYDEVLKTFMADQKIEDAYQTLKPVLDALHERFINDSLELDEVKNIDFSEYLEKYRDKDLKNKDFEALERKLRVKFKKLYEKVGENWIAKAGFNDKGKPVFKEKSFKILTEKGVLDFIKKNADNFTFVSKEQLEGAIKVFENFFTYFTGFNRNRENYYETGKEAYTAVATRIIHDNLPKFCDNLIVFESRSKDYLGAYELLKGRGLSLVTKEGRPLMPIVDNIFRIDYFRKCLSQAQIEEYNKQIGNANFVVNLYNQVFSGNSDFKKLLSFKLLYKQIGCGKRRSFFVELTHDTIAEADKARGEGKKAFSVEELVLLAKQAGEIYFQGKSDDGIINTVPELLDYLEKKSDYRGIYWSKTAINTVSGKYFANWFDLKNKFKEAKVFEKNKKGSEEEVKIPDVVELEDLFSVLNEWSDWKTTLFKSSIMQDAKKRELIEKSPNASTALLSLLFADIKDHAECFIDEANEVSQVKDFKKEENKILIKLWMDHALVVCKMLKYFLVREDKVKKVKGALMDATIVEALNNILSGQVEVDKNGKKIAAEVDWFEWYNYFRNYLTKKPQDDAKKNKLKLNFECASLLGGWSDGQEKEKGAVLLKKDNFYYLGILKKKNLFDTEKENNPVYQNPTFSAGRLILANLKFSTLAGKGFLYEFGKAYGDMGAEDPDKAIKCLKKIIKDRYEEKYPLLARIAATHYSDKKVFDKDIQETLKECYVCRFEPVSWIKVEEYVDDGELYLFQIYSKDFATNNTGKNDLQTLYWKAIFDEYSSFQLNGRGEIFYRKQAIKEKIVKVGYEGKPWVIADKRFTLEEGKFAFHCPIKINYKAKNYSQPKFAFSEINKIINDGFGKFSNIYFLGIDRGEKHLAYYCLVDQNSKIIEQGSLNMPFVDASGNPRMVKTERKFIQEDGKEQLEIVECKDYNELLEARAGNRDYARKNWQVIGTIKELKEGYISQVVHKIAGLCANKEHPVFIVLEDLNTGFKRGRQKIEKSVYQKLELALAKKLNFLVDKNAKLGEVGSVTKALQLTPPVNHYADIKNKKQVGVMLYTRANYTSQTDPKTGWRKSVYLKPGSEENIKNQILEQFSDILFDGKDFVFVYADANTGTEWKLYSGINGISLDRFTVKKGVKNEWCIEKQDIVEILEGVFSKFEKNRSFLSQIKDEGRELTKISDHTAWESLRFAIELIQKIRNTGITSRDNDFLLSPVRDSAGNHFDSRVYLDKEQRGEKIDLPSCGDANGAYNIARKGIIMWEHINRGYRPYVSDDEWDAWLAGVTNWEKWREGNEKLLKYNK

SEQ ID NO: 16 - protein, 1267 aa, synthetic construct
MKKFIAKYNISKTLRFELKPIGKTLENIQKNGLISKDEMLAENYKEMKKTIDEFHKEFIHKTLSNASLDKENLKEIAELYTAPAEKRKEDNYKNQLEAYQKALRKEIVDRFKNDELFKNLFSEKLIKNHLQGFAQRKGLRFYSEFNNFTTYFVGFHQNRKNIYTDQAHSTGIAYRLIHENLPKFIHNISVFEKIQEKKIPLTILENQFMSKLGNDTLADVFRLEYYNCLLTQEGIDFINQLIGGYVNENQEKIQGLNEYINLYNQQQKDKKDRLPKMKMLYKQILSHRESISFLPQSFKESQEVLDAVRDYYLSNLLEYRAEDKEEPENILEKIQERLSHIADYDLSKIYIRNDKAITDISQTIFGHWGLIHSALEFDFRQRNVGKKTTQKQEENVEKYLKSDYYSIAEIENALISYKESTQNEKLVEVFNKTSHPIADYFKTYFKAKEKSKEGKEYTLIANIQAKYTCVEGLLNTDYPKDKKLHTDKDNLRNLKAFLDAILEFLHFVKPLILRKNDFLEKDENFYSVLETYYTQLQLLVPLYNKVRNFATQKPYSTEKIKLNFENSTLLNGWDKNKESDNTCVLFRKGKDFYLGIMNKKYNKIFENVPKPKSDTTFLKMEYKLLPGANKMLPKVFFSKKNIAYYAPDSEILRIRNHSTHTKDGEPQKGYEKQAFKLEDCHKMIDFFKSSIEKHPEWKNFNFIFSDTQHYQSIDEFYKEVEELGYSIGFVEVDEEYINQCVNEGKLYLFQIYNKDFSPNSKGKPNLHTLYWKAIFDPENLKDVIYKLNGEAEVFYRKKSISADNIIVHKAQKPVENKNPLNTKKYSTFEYDIIKDRHYTKDKFLFHVPITLNFKSKKGKENINQEVLDYLKENPDVNIIGIDRGERHLLYLTIINSKGEILHQESLNKVKNEVYGIEIETDYHLLLNKKEEERNKARKEWGTIENIKELKEGYISTITHKIAKLMVQYNAIVVMEDLNSGFKRGRIKVEKQVYQKFEKMLIEKLNYLVFKDVPNYEPGGLYNALQLTNKFESFEKLGKQSGFLFYIPAWNTSKIDPTTGFVNLFGGIKYESIHQAVNFFEKFQSIRYNTKEAYFEFAFDYNDFTTKAEGTKTQWTVCTHGERIINFRNTEKNSEWDSKTVQPTQEIKNLLNEYHIAYEQGQDIKPAITAQNDKKFFEELLKQFKNTLQMRNSVINSDVDYLISPVMNAKGEFFDTRKTDSTLPQNADANGAYHIAKKGLLYVQQIIDGVKKTDLTNKKWLKFVQNNL

SEQ ID NO: 17 - protein, 1241 aa, synthetic construct
MGFKYMQAYRCLKKFVNQYTLQKTLRFELVPNPITKKILEIESAKKSFLNDRERNKAREELKEMLKELDNEFIQEALRDFQFSKQDLQELWEKYKKDKKSIQDKDLKKLESKIEKRFSCFSSSKYGKIKFTKQNILSEKVIKILKEKYKDNQQKQKIIDLFDRFTTYITDLKQNRENYYKAEGKTSQFATRAVRDNLIRFFDNKFIFENKYSQNFGFSEEEKKIFLLSYYNGCLLQKGIDRYNELVGNLNSKINQYILKHKNNHDFKKSDYPFFKKLDKQIVSSKDVRKSIEINDDQELIECLDCFYRFAQGIIPAIKKLWQKFFEPERELGKIYLPRNKANRLSYYFFGDWSLLSNVYGRGEKKQEFVSFRKIKEYLQDFDNLEFRKLTANQDIIKKEKDNFANFIDAWKYEGEVLFSDKKEKSIFIFPEGKKEFKSLVDLEKGFLEKVNWFRKKIENKEKISDKEESKFKMAVKDYLDRVLDIFRLVDNFYLKEEQRNKIPVEELDSEFYKFFDEYQEKISDFNPYKYYDEFRNYLTKRNVVADKIRIAFDEGQLLGGWDLNKEKKKLGLIFRKNGNYYLGIINKKKDKNIFDKNKHPEAFVESSKWEKMEYKLLGDLKRQIPRIAFSEKNRDIFGYNSEIQKIKEEYDQLQNSKKENRDNWHKKFNKRKLIKLISYYQKVLEFHPERYKENYKLSWKKPKEYNSLGEFYDDIDKQMYRIKFVNVSDDYLEEKVVKGELYLFQIFNKDFSSYKESNSKENLHTIYFRELFSEENLKNPIFKLSGGAQIFFRDKLEEYQIKTKKDKKGKTVIDRPRYAKDKILFHLPIELNYKAGKANKFNQKINKFLASNPDIKILGIDRGEKHLLYYSLIDTNGKILKSGSLNKIKIGDKEIDFHQKLRERAKEREEARKSWQSITQIKNLKKGYISLVIKKIADLAINENAIIVLEDLNTPFKQKRGGIFEASIYQQFEKALIEKLNFYVDKEKKNHRRAIQLTAPFTTFERIDKQSGIIFYVPANYTSVTCPECGWRQRLYLKYKNINQAVKDFERIKVIYEPTKDRFVFEYGFEVKDNKKKNSFKGIDKIYSDVERLWWNPKYNENGKYGRIEKLGLGEITKELKNVFKEMEIDYHQEGNLSEQLTSKTDKKYSKHWRELYWLFRMIAQIRNLDRENDEENKDFILCPACHFDSRDKNTLIPNGDANGGYNIARKGIILLQRITANPQKPILTISNRDWDEFVRR

SEQ ID NO: 18 - protein, 1205 aa, synthetic construct
MIESKLSKYLNLYSISKTLRFELKPVGDTLKNFKSDNILEKDKEKALKSKEVKKILDEYYKEKIDEILNNNLFEKSILDEYIKNQNIKENIKIIKDIMKSTIVSRFESKNILENGKEVTTLKLLTSELVFKSLDIMLEKANRIEDLKKIKLFDKYTTYYTDFFKNREFIFKGKDQGSIAYRLIDENLDIFMDNLNNYNQNVPEEIKNKFNNKSIDFLVDIESFNSVLTQKGIELYNLLISGQTLEKENKVQGLNELINLHNQLNKTKIPLMKILNKQILSDKQTFSFVLDIIEDDETVFALIDEYIKNTCFINLFENKDINYDEVYITNNQNITLLSKKLLSNWNEIKNLLYSWYEKNIDHKTENKNFLEKRTRFFKNTDHFSIKFLSNLTDNRVNSSFFITESNDYILKIKSSIDEYRKIIRTNIKNLKKSEEAVLKIKALLDSIKEYQEFLKQFLMSDIRVQTLKSLEEGVNSLSSKDSLFYNDFDNSYNNIFSIVAIYDKIRNYLTKKPFKTDKIKINFNCSTLLNGWDLNKEKANLGVIFKKYDINIKKDIYFLGIIKDKKIFDKLKETDEQNCYEKMEYKLFPGANKMLPKIFISAKAYRANLSEEFLKNYSEKKHTKSNLDEKFLAEYILYMQKSLNDYYKDEFNFKFNKAESYKQIDEFYRDVEEQGYKLKFKKISESYINKCILENKLYLFQIYCKDFSNYSHGLPNTQTIYLQSLFSNDNLNNPYMKLLGNAEIFYREKSLEIIETHKANEQLQNKNVKNLKKKSQFNYSLIKDKRYTEDKFLFHFPITINYKSGNIRNINQLINANINDFEHVIGIDRGERNLVYIVVSDLKGNIKDQISLNEIVNSHNENEYRTDYNDLLEKKAKGRDEEKKNWSEIENIKELKEGYISQVVSKIKDLALKYNALIVLENLNFGFKNSRIKVEKQVYQRLETQLINKLNYVIDKKDSRTYLNGIQLTNPINNLSEIGSQSGIVIYIPAWNTSKIDPTTGFVNLIYGLKYKNIADSKEFISKIKDIRFNGKYYEFDIDFGEFNNKLKETKLNWTICTYGTRVETRRDEKNNNEWVSKEVDLTKRFEELLKKYNIHSDFKNQILLQNSSDFFEEFIHIFKLILQIRNSIVGTKIDYMISPVKNPSGKFFDTREGIKTLPLDADANGAYNIARKGIMIIDRIKNTTIGEKIDYKISNEEYLLYLQNQ

SEQ ID NO: 19 - protein, 1366 aa, synthetic construct
CTSRRRIAMKSIFDNFTGLYSLSKTLRFELKPEVETKKRFDNWIKEVESNNESTAENLIAKDYKIFNAYKTLKPILDSIHEQYITIALQSSVAKKIDFTCFLPEYSDKTKKLSKIEEKLRKEIGETFKVSEEFFFEKIKAVEPKVKSKSKDSILKDQYVLTYIEKNIDLFTSPSLSEDELKQHLKAFKGFFTYFSGYNINRENYYDCSSEKNTAIATRIVNDNLPIFCDNKIRFEKNKKCYLEAYQVLKNAGIETKIKLSDDTFSEAKPISEDIFEMSYYNNCLSQSEIETYNKMIGNANLIINLYNQHAGASDNNFKRLPVFEQLHKQIGCGKKKSLFFSLKKDKETELSLEERQSEEILSVEKLLNVTAEAGNAIFNGSKESNAYSINFFTDFLLEQTDWHGIYWSDTATAYISGRYLLNWHELKDQLKNYSSCASFDKKREEQVKLNEAVELAGLFEVIDKENVENIFKTSVLEKYSDVIKTDEPVSKNLIKIICADLKCSLDYFNTNSKKILSMDSYKTKSAYADEDDASVMQIKNWYDSIKEILRIVGLFKVKKNKMKGSLSIPEFENCVDAILSPEGIYWNKWYDGIRNYLTKKPQDDVKENMLKLNFGSSSLLGGWSDGQEKVKISTILKFENDIFLCILKNKKFFDTGKKENPVYKGEGGFRLILRNLKFQTLAGKGFVSAYKQGYSEMGKENPQKAMECLIAFIKENYVKKYPALKKIVEKKYTSKKEFDAAIQDVLKECYECSFVPIDWNIIQTAEKKGDIFLFKTSSKDFKEKSSGKKDLQTIYWESVLSENSPHQLCAGAEIFMRKPIQAKSPVVHKAGDILINKKFSDNKTVPDEFYKELMKFVSNIKQPVENEQVVKDLLFKSELAKNNTKEYLDKVKFKIAKHYIEKDRRFYGETKYFFHCPIKLNYNQKSYSKPEYAIKEMNDLINEGLSKQEAVHFIGLDRGEKHLVYLCELDENCNIIECRSLNVINGTDYMLKLQEKSDNRQKSRQNWQKIDNISNLKDGYTSHVVHAVTEKAVRTPSYIVLEDLSRQMMQGRQKIEKSLYQKFEVALAKKLNFIVEKDIPQGEPGSVSLPLQLTPPLNTFKDIENKKQFGIMMYTRANYTSVTDPVTGWRKTIYIKNGSEEDIKNQILEKFSDFGFDGKDYYFEYKDANTEYTWRLYSGKDGEPLPRFQNKKQLQNDKNIWVPEKIDVVEILDKLFSDFNKEKSFKEQIMNGINLKKIDGRSETAWQSLLYAINIIQQIRNSGISEKDNNFLYSPVRNNNGIHFDTRIQDNKTLPADADANGAYNIARKGIIMYNHIQQWKKEGALEKDLDLFVSDEEWDLWLLNKKAWKEKLTYFASKSKKQKKQ

SEQ ID NO: 20 - protein, 1324 aa, synthetic construct
MEKEKTKSIFDNFTRQYSLSKTLRFELKPIGKTRENMNANLEYDENLRTFLADQKIEDAYQILKPVFDKIHEEFITKSLESAAAKEIDFSEYFNLYKKQKNEQNKDERKKLDKLIQNEEKKLRKEFEKFYLIAGEEFKIIAGKDSNGKDILKKNGYKVLTEAGILAYIKNKLLANPAWLDDLKIPEVKVLVENKEKNKEKVEKALEQMKRFFTYFGGFNQNRENYYDVSANKNGEGKSTAVATRVIEENLPKFTDNLLSYEKNKDEYDKVFLKLQNDNKELRLRNPRWDGIDKNTEFIPAYDIKNDGTSFAIGNFVNCFSQSEIDAYNAKVANVNYLVNLYYQQTKNKLPKFKILFKQIGCGEKKDFIKLIKNNAQLKAVLEKAKLAGEKYLINDKDKDAVGAFMDYLLNLDDFKNIYWSDKAMNTISGKYFGNWGNLKDKLLKAKVFNKDKNSGEAKIPQAVSLSDLFEVLDAERDWQGKGILFRENFKENNEEKQDIIKNSRTPHDALLKMICKDIENLAQKFVDGADEILKIKEGDYQKDESKIAIKTWLDSALFVGQILKYWRVKARFSVDSNFTDILDKVKVSETVQDYDVVRNYLTQKPQNKLGRLKLNFENSTLAGGWDVNKEKDNACLILQDKKGKQYLVVTECSGAGIFEQTAQNSLYAPDASGWTKMNYKLIPGPNKMLPKVLFFSSKWIKANPTPDNIKTIYEKGTFKKGDNFSQNDLHALIDFYKDQLKKYPLPEESWNRIFDFDFSDTKKYKSIDQFYTEVERQGYKIEFVPINKGKIDELVEGGRIYLFEIKSKDSNLKNGKEKTSAKDLQTIYWNEIFGDAENKPKLNGGAEIFYRSALEEADLKKKNWKNKEIIENFRFSKEKFILHCPITLNPCLKNNRINDLVNKNFAKSKNQLFLGIDRGEKHLAYYSLVNRKGQILEQGSFNTINGQDYWDKLDKKQGDRDEARKNWTTIGNIKDLKDGYISQVVRKIVDLAVYNEGDRKKGFRETPAFIVLEDLNIGFKRGRQKIEKQVYQKLELALAKKLNFLVDKNAKVGEMASVDNALQFTPAVREFSDIKGKQFGIMLYTNPSFTSKTDPITGWRQSIFIKKGSVKYIQGQIMGSFSDFGFNGKDYYFGYEDVNTGKEWTLYSGKNGRELDRYRDKFSEKEGKKHWNPEKIEIVKKLEKIFDGFDKNKSFKEQIKSGKQINKIDEHTAWESLRFTIDVIQQIRNTGNNEKDNDFILSPVMNDNGDFFDSRDIKDNEKFPQNGDGNGAYNIACKGIIMSEHIKRGLNLFIRSEEWDAWLAGEKIWEKYIAKNLKIRKKVS

SEQ ID NO: 21 - protein, 1214 aa, synthetic construct
MKQFINLYPVQKTLRFELRPVGETMKNIEEKGILTADEHRAYAYGKVKKFIDEYHKAIITKNLSGLVLDSSALNDVLIFSGKTDVTLRNKANKAMEKLRKQIADSFDLKSLFSEKVIKIDLPAFLNDDDKNQLVAEFGNFTTYFSGFNENRRNMYTAEPKATGIAYRLINDNLPKFIGNIKAFEAVKEPLANQIEELKSQFEPMLEGIQLEDLFSDVTDYSVLLTQEMITLYNTLIGGKSEGSVKIKGLNEYINLYNQTHKDKLPKFVSLYKQILSDRDSLSWLPETFKSDSELMESIESFHRLFESRISNGDDETISLKELLVSIADYDLKGIYMSTGTGLTSVMKSHTGDWNTLNKAMEEDFDRKYPCTKEKDMPKHLEKRAKYIKSFDSLSIADINVYIGEEKALQSYFMTLGASSENGKTLFERIEENYCVIKDLLNTEYSKNLTQQDDDIKKIKTYLDSIKELQWFIKPLNGTGSESGRDGRFYGDFNELYGLLDKVTPLYNMVRNYVTRKPYSEEKIKLNFDNSTLLAGWDVNKERDNSGVILRKDGLYYLAIMDKKFNKSFDMDSYPCEGECYEKMDYKLLPGPNKMLPKVFFSKSRISEFTPSQSILDHYSRGTHKKGEDFSIDDCHKLIDFFKASIEKHEDWNKFGFHFSDTSTYNDISGFYREVEFQGYKISFRNVSCTYISRLVSEGKLYLFKIYNKDFSTFSKGTPNMHTLYWKALFSEDNLKDVVYKLNGDAEVFFRRKSIQYDDEKMKKGFHHEQLKDKFSYPIIKDRRYTVDKFQLHVPITMNFKSYGIDNINMMVREYIKDGEIDHIIGIDRGERNLLYISMIDLDGNIVKQLSLNEIINRHNSCEYRTDYHSLLAKKEGDRLDARKNWKTIENIKELKEGYLSQVIHIIAQMMVEYNAIVVLEDLNSGFMRGRQKVEKSVYQQFEHMLIDKLNYLVDKKRPVMEEGGLMRAYQLTSKFESFQKLGRQSGFLFYVPAWNTSKIDPVTGFTNFISTKYENIDSARELLGKFKDIRYNAEKGWFEFDIDDYSSFNPKAVGRQEWTLCTYGTRIRTFRNERKNNEWDSEEIHLTEEFKRLFGSFRIDYTENLLGSILIQSSKEFFSIILSLLGLTLQLRNSKTGTDEDYIISPIAGPDGRFFDSREYDSKDSTLPSDADANGAYNIARKGLWTLRQISLADDLSKVNLAISNEGWLKFVQK

SEQ ID NO: 22 - protein, 1371 aa, synthetic construct; identical to SEQ ID NO: 10

SEQ ID NO: 23 - protein, 1262 aa, synthetic construct
MENMANSLKDFTNIYQLSKTLRFELKPIGKTEEHINRKLIIMHDEKRGEDYKSVTKLIDDYHRKFIHETLDPAHFDWNPLAEALIQSGSKNNKALPAEQKEMREKIISMFTSQAVYKKLFKKELFSELLPEMIKSELVSDLEKQAQLDAVKSFDKFSTYFTGFHENRKNIYSKKDTSTSIAFRIVHQNFPKFLANVRAYTLIKERAPEVIDKAQKELSGILGGKTLDDIFSIESFNNVLTQDKIDYYNQIIGGVSGKAGDKKLRGVNEFSNLYRQQHPEVASLRIKMVPLYKQILSDRTTLSFVPEALKDDEQAINAVDGLRSELERNDIFNRIKRLFGKNNLYSLDKIWIKNSSISAFSNELFKNWSFIEDALKEFKENEFNGARSAGKKAEKWLKSKYFSFADIDAAVKSYSEQVSADISSAPSASYFAKFTNLIETAAENGRKFSYFAAESKAFRGDDGKTEIIKAYLDSLNDILHCLKPFETEDISDIDTEFYSAFAEIYDSVKDVIPVYNAVRNYTTQKPFSTEKFKLNFENPALAKGWDKNKEQNNTAIILMKDGKYYLGVIDKNNKLRADDLADDGSAYGYMKMNYKFIPTPHMELPKVFLPKRAPKRYNPSREILLIKENKTFIKDKNFNRTDCHKLIDFFKDSINKHKDWRTFGFDFSDTDSYEDISDFYMEVQDQGYKLTFTRLSAEKIDKWVEEGRLFLFQIYNKDFADGAQGSPNLHTLYWKAIFSEENLKDVVLKLNGEAELFFRRKSIDKPAVHAKGSMKVNRRDIDGNPIDEGTYVEICGYANGKRDMASLNAGARGLIESGLVRITEVKHELVKDKRYTIDKYFFHVPFTINFKAQGQGNINSDVNLFLRNNKDVNIIGIDRGERNLVYVSLIDRDGHIKLQKDFNIIGGMDYHAKLNQKEKERDTARKSWKTIGTIKELKEGYLSQVVHEIVRLAVDNNAVIVMEDLNIGFKRGRFKVEKQVYQKFEKMLIDKLNYLVFKDAGYDAPCGILKGLQLTEKFESFTKLGKQCGIIFYIPAGYTSKIDPTTGFVNLFNINDVSSKEKQKDFIGKLDSIRFDAKRDMFTFEFDYDKFRTYQTSYRKKWAVWTNGKRIVREKDKDGKFRMNDRLLTEDMKNILNKYALAYKAGEDILPDVISRDKSLASEIFYVFKNTLQMRNSKRDTGEDFIISPVLNAKGRFFDSRKTDAALPIDADANGAYHIALKGSLVLDAIDEKLKEDGRIDYKDMAVSNPKWFEFMQTRKFDF

SEQ ID NO: 24 - protein, 1243 aa, synthetic construct
MVAFVDEFVGQYPVSKTLRFEARPVPETKKWLESDQCSVLFNDQKRNEYYGVLKELLDDYYRAYIEDTLAAFTLDKALLENAYGLYCNRDNNAFSSCCEKLRKDLVKAFANLKDYLLGSDQLKDLVKLKAKVADSAGKSKKKIEVDSRLLAWLKNNAKYSAEDRVKYCEAIEAFEGFVTYLTNYKQARENMFSSEDKSTAIAFRVIDQNMVTYFGNIRIYEKIKAKYPELYSALKGFEKFFYPAGYSEILSQNKIDEYNYKCIGRPVEDADYKGVNSIINEYRQKKGIKSRELPVMSMLYKQILSDRDNDFMSEVITSNEEAIECAKAGYKKSYILFNELLQLFNNSFIEENYEYIYIKTQPLTELSQSLFGDWSVLRNALDNAKYDKEIINLAELVRYFSEYCQILDADDAVKIKNKFILEDYFVQDTLEAALPDLDKITQYKTYLDNMLQVIRKYRLFSMYNGRKKLDVPEKGMDFSNEFNDIYDKLSEFSILYDRIRNFATKRPYSDEKMKLSFNMPTMLAGWDYNNETANGCFLFIKDGKYYLGVADSKSKNIFDLKKNPHLWDKYSFENAFYKIKYKQISGAAKMLPKTFLPSKGKPLSEEKKKKYGHLVTDRIIEIRDNGLYMKSAGDRKAVIEWIDFMKSAIAIHPEWNEYFKFHFKNTEEYASANEFYEDMDKQTYSLEKVKIPAAYVDELVSQHKLYLFQLYTKDFSDKKKKKGTDNLHTMYWNGVFSDENLKAIAEGTKPIIKLNGEAEMFMRNPSIPRKITHERNKPIANKNPLNNKKESLFSYDLIKDKRYTERKFYFHCPITLNFRAAKPTKYNEKINRFVENNPDVCIIGIDRGERHLLYYTVINQTGDILEQGSLNKISSSYTNNKGEKVNKETDYHALLDRKEKGKHAAQQAWETIENIKELKAGYLSQVVYKLTRLMLKYNAVIVLENLNVGFKRGRTKVEKQVYQKFEKAMIDKLNYLVFKERGYEMNGSYAKGLQLTDKFESFEKIDIDKQTGCIYYVIQSYTSHIDPKTGFVNLLNSKLKYESVSKAQETIRKFDRISYNAKADYFEFSFDYGKFGIDMFRNEWVVCTCGDWRWEYSAKDRETKAYCVTDRLKELFKSHGIDYVGGENIVSKIADVADKRFLSTLLFYLRLVFKMRYTVSGTDNENDFILSPVEYAPGKFFDSREAAATEPMNADANGAYHIALKGLMTIRGIENGKLHKFGKGGEKAAWFKFMQNQEYKNNG

SEQ ID NO: 25 - protein, 1324 aa, synthetic construct
MLNKFTNQYSLSKTLRFELKPVGETAGYLDDFKSQHLKEFVATDKQRADDYLIIKELIDDYHRAYIEEKLSAPVDPKTGELFLNSQDFEDAYSYYQQLKVDSKDPKHRETWEKTQAELRKKLVKAFVGKADLFGEKLIKQHLPEQLKKEGRWKENKKAVENFNKFTTYFSGFHENRKNMYTDKDQSTAIAFRLMNENLPRFFNNCDKYKKLKEKYPELLFENNKDILAKLGVDSLNDVFTPSFFINLFTQTGIDNYQLLLGGKSEDTGEKLKGLNEQINLFRQQKQKEAREQAQKENKKAKKITDLTGFTGLYKQILSDRQSASFILDEFENDRELLTKLNEFITLATKPEGTLEKLENALNHLNEIDPQKVFIKTAGLTDISQTLFHSYQIITTALHEFAENNLKTKKERENYLKQKAFSLAELDERLVDYIQDLEDSDPLHQQLQALNDPLQPIKGYLQEAITRAQQETDERPNLEQTIKEVEPLLTLKELSKNRQAPKKEGDKGGKGFQQIQKIQKMLDAFMAVSHALKPLHLVDGRKPIDMPEMDSGFYGEFSEAYEEYNQSCITLYNKSRNHLTKKPFKTDKIKINFDSPTLLKGWDANKESDNASLLFEKDGLYYLGIMHPKHKRLFDYIIGIDDIGNENKTKAKTDLYNQITTNKSGYRKIVYKLLPGANKMLPKVFFSRSRIAFFAPSDEVQRIRNTASHSKNGKPQKGFKKADFNLKDCHTMIDFFKDSINKHPEWKQFGFKFSPTESYEDLSGFYREVEQQGYRIDFHTIKESYIDECIKQGKLFLFQIYNKDFSTYSKGKPNLHTLYWKGLFEAENLKDTVLKLNGEAEIFYRKHSIKPNDIITHRANETIENKSKNYDKKTSTFAYDIIKDKRYTQDKFQFHVPITLNFKAPKAPGVSHFNNQINKAVAKSKDTHIIGIDRGERHLLYYTVINQKGEIIEQDTLNKIKAHKDYEVDYQQKLHDKETERQKARRSWSSVENIKELKAGYLSHVVHKLAVLIKEHNAIVCLEDLNFGFKRGRFKVEKQVYQKFEKTLIDKLNYLVFKDAEENEAGHYLKAYQLTAPFESFEKMGKQSGILYYVQAAYTSKIDPATGFINFLYTRYQSLSRSKEFFESFDFIRYNQAQDYFEFSFDYKNFPIRQNLKNYQTRWLACTYGDERYANKRNKQNQWESEIVNVTQELKTLFNKASIDFQNGKDLKDAIAQTKDTKFYKKLYWLLGLTLSLRHSKTGTEIDFILSPIQGKDGKFFDSREASNKHPKDADANGAYNIALKGLWNLQQIKQHDWNTEKPKKLNLNMKNEDWFGFIAERASL

SEQ ID NO: 26 - protein, 1301 aa, synthetic construct
MSQDNQKQFKTFQDFTNLYELSKTLRFELRPIGKTKELLEEQRVFQKDKKILENYKIAKKYFDKLHKKFINDALNNADLNFEEYKKAYFAYIKKNKDNRKKEFAELKKQETNLRKEVKSLFNHCAKSWKEKYNKVDVETKKDDLGILFEKETLNILQYEFRAEQYPDVEYADEKGEKKNIFESFKGFTTYFTNFHNSRKNFYKDDGTVSAIPTRIINDNLRKFIENSEIYSHTQNKIEFTNEEKKIFELDYYNKCFLQGGIDEYNKIIGDINSKINKYRQDKKDRQNKLPFLKILYKQILEDIGKQETKQNGFIEISNNGEVFEVLEKFIEHNENNNKKFKEIFSSFINNQNEYELDKIYLASRFVNTISNKWFNGWELFRGLILEKLNDNKSKSKQKKKLPYFIPFSQIKEILARTDIKAEDLFKKDILNKVGYTNQNVYDVFISIWKNEFKENVKKYEETLSKVKAMIKEDKEYTNKKEKLENGVEVEVQKKKIENYADSALAIYRMMKYFLLEKGKSSIDMQGDTDNKFYNDYDNAMENAKTWLYYNEFRNYLTKKPFNEDKIKLNFENGKLFDGWDQNKEPDNFGVILKKDNRYYLGVAEKESANKIFRKKKGSSSEKIQEAYNFNGGYYEKMEYKQVPGLNKMIPKCLIAPYFKKRGNKKIIDRSKPGAKKLEKLGINPDDNFLLNYIENKHAKGDNFDREFFNEYVAFVKNSIPKYPDWECFDFNLKEIKDYDNLMDFYKDLERNSWKVWFEKVSEKYINEKIENGELYLFQIYNKDFSEKRTGNKNLHTIYFESIFNEENIKDPVIKLNGQAEIFFREGNLKIEEELRTKKRNKKPHKIIKYKRYTENKILFHCPIKLNFAKSDNKINLKVNELLANNKDINIIGIDRGEKHLAYYSVIDQDGKIIDIGSLNKINNVDYLAKLDEKEKSIEKQQKSWQTIENIKELKQGYISQVIKKICDLAIKYNAIIVFEDLNSGFKRGRQKIKKQIYQKLELALINKLNYLVNKNEENGKAGHFLSAYQLASKIDNFKDIGKQSGIVFYTQASYTSKTCPKCGFRKNITLKYINDKQVKDDLKKLKIKPVDDYFKIEYKLSDFSENNKKKKNKDNELLAEYQRKNDFIIYSNVKRMRWHNINTKLAKRELDGEKIIKKFPPKGVIKEYDINKCLKRMFKGKINIEKGDIIDQLIKANFNKDYYWELMYFLNLILEIRNSVSETDIDYIQCSKCGFHSDGGFQNKEFNGDANGAYNIARKGRLILEKINQHQDLSNLKTGDLAINIEEWDKAVNNWDKFVQK

SEQ ID NO: 27 - protein, 1335 aa, synthetic construct
MKSLAQFQNLYALQKTLRFELKPEGHTRETFNRWLEEIEKEQASENENIVYQDLLRAKKYEKIKIILDEYHKDFIEQALAYANLTELEKYEELYRKSNRTSEEEEEFENTKESLRKQIANIFIKNPNKTVQERWKFLFSKKLIQNELIVWVKGNYELLSEKLKNEFPDESSIISTIEDFKYFTTYFRNYHENRKNLYSNEDKFSTIAHRLIHENLPKFIDNIAIYQKAKAVLNINEVEKELGLPEDTLDKIFSLDFFSKALTQKGIDQYNYFLGGKTENEVKKIKGLNEFINLYNQQQQDKNQRLPFLKVLYKLPLFERTSTSFRFEPIENDRDLIERIGKFYYNDLKQYRDDSQGDTTDILSGINTLLRHVHDYRDGLYVNGGITLTQISQKIFGSWSYINNALAYFYDTYIDASGVDHQGERKPKKQKQIQEKTKWLKQKQFPVILVEKALSEYKSIETNEDLKTRISDTTLCDFFKRCGNDDNGQDLFDRIEARLREKNEEGYSLEDLLKKEFTTERKLMQDKTKTLLIKNFLDVIQGDKDDITAGLLHFVKCLIPRTEISEKNELFYSGMEKYLNILSEVTPLYNKARNYLTQKPYSIEKVKLNFENSTLLDGWDENEESDNSCVLLRKRGYYYLGIMNKKHNMIFDRKIYPKATEGEAYYEKMIYKLLPGAYKMLPKVFFSEKNIDYFKPSEEILRIRNTASYSKNGQPQEGYQKASFSIEDCRKYIDFFKKCIANHWDWQKFNFNFSPTEYYQSIDEFYREIERQGYKIDFVKIPESYINQLIKENKLYLFKIYNKDFSEKKKSKGKDNLHTLYWKMLFDEKNLKDVVLKLNGEAEVFFRQKSILYNEEIWNKGHHYSELKDRFSYPIISNKRYAEDKFFLHVPITLNFKADGINNVNNMVNEFIKDNRDIHIIGIDRGERHLLYVSVINQKGDIVEQCSLNEIVTEYNGKIFKKNYHEELDNLEKERDRARKDWQTIANIKELKEGYLSHVIHKISKLILKYNAIVVMEDLNSGFKRGRQKVEKQVYQNFEKQLIEKLNYLVLKESNVDEPGGVLRAYQLANKFETFKKLGKQSGIIFYVPAAYTSAIDPVTGYIQYLYPLKQADSVEKARKFYSQFKRISYNPHKQWFEFSFDYNDFNIIYHGKSSWTICTTNTERYMWNRLLNNGHGGEELVYVTNELELLFGEYNIIYGDGKDIKQQITDVQDIDVDRTAKQFYKRINELLNLTLKLRHNNGKKGADEEDYILSPVEPYFDSRFESRKPSMQQTLPINADANGAFNIARKGLLLLERLNQLGVEEFEKTKKSNNKKTQWLPHELWVEYAQNHTRK

SEQ ID NO: 28 - protein, 1281 aa, synthetic construct
MLKSYDYFTKLYSLQKTLRFELKPIGKTLEHIKNSGIIESDETLEEQYAIVKNIIDKLHRKHIDEALSLVDFTKHLDTLKTFQELYLKRGKTDKEKEELEKLSADLRKLIVSYLKGNVKEKTQHNLNPIKERFEILFGKELFTNEEFFLLAENEKEKKAIQAFKGFTTYFKGFQENRKNMYSEEGNSTSIAYRIINENLPLFIENIARFQKVMSTIEKTTIKKLEQNLKTELKKHNLPGIFTIEYFNNVLTQEGISRYNTIIGGKTTHEGVKIQGLNEIINLYNQQSKDVKLPILKPLHKQILSEEYSTSFKIKAFENDNEVLKAIDTFWNEHIEKSIHPVTGNKFNILSKIENLCDQLQKYKDKDLEKLFIERKNLSTVSHQVYGQWNIIRDALRMHLEMNNKNIKEKDIDKYLDNDAFSWKEIKDSIKIYKEHVEDAKELNENGIIKYFSAMSINEEDDEKEYSISLIKNINEKYNNVKSILQEDRTGKSDLHQDKEKVGIIKEFLDSLKQLQWFLRLLYVTVPLDEKDYEFYNELEVYYEALLPLNSLYNKVRNYMTRKPYSVEKFKLNFNSPTLLDGWDKNKETANLSIILRKNGKYYLGIMNKENNTIFEYYPGTKSNDYYEKMIYKLLPGPNKMLPKVFFSKKGLEYYNPPKEILNIYEKGEFKKDKSGNFKKESLHTLIDFYKEAIAKNEDWEVFNFKFKNTKEYEDISQFYRDVEEQGYLITFEKVDANYVDKLVKEGKLYLFQIYNKDFSENKKSKGNPNLHTIYWKGLYDSENLKNVVYKLNGEAEVFYRKKSIDYPEEIYNHGHHKEELLGKFNYPIIKDRRYTQDKFLFHVPITMNFISKEEKRVNQLACEYLSATKEDVHIIGIDRGERHLLYLSLIDKEGNIKKQLSLNTIKNENYDKEIDYRVKLDEKEKKRDEARKNWDVIENIKELKEGYMSQVIHIIAKMMVEEKAILIMEDLNIGFKRGRFKVEKQVYQKFEKMLIDKLNYLVFKNKNPLEPGGSLNAYQLTSKFDSFKKLGKQSGFIFYVPSAYTSKIDPTTGFYNFIQVDVPNLEKGKEFFSKFEKIIYNTKEDYFEFHCKYGKFVSEPKNKDNDRKTKESLTYYNAIKDTVWVVCSTNHERYKIVRNKAGYYESHPVDVTKNLKDIFSQANINYNEGKDIKPIIIESNNAKLLKSIAEQLKLILAMRYNNGKHGDDEKDYILSPVKNKQGKFFCTLDGNQTLPINADANGAYNIALKGLLLIEKIKKQQGKIKDLYISNLEWFMFMMSR

SEQ ID NO: 29 - protein, 16 aa, synthetic construct
KRPAATKKAGQAKKKK

SEQ ID NO: 30 - protein, 7 aa, synthetic construct
PKKKRKV

SEQ ID NO: 31 - protein, 9 aa, synthetic construct
LPPLERLTL

SEQ ID NO: 32 - DNA, 20 bp, synthetic construct
  source 1..20: mol_type = other DNA
tattaaatactcgtattgct

SEQ ID NO: 33 - DNA, 330 bp, synthetic construct
  source 1..330: mol_type = other DNA
  misc_difference 31: note = a, c, t, g, unknown, or other
  misc_difference 32: note = a, c, t, g, unknown, or other
  misc_difference 33: note = a, c, t, g, unknown, or other
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 58: note = a, c, t, g, unknown, or other
  misc_difference 59: note = a, c, t, g, unknown, or other
  misc_difference 60: note = a, c, t, g, unknown, or other
  misc_difference 61: note = a, c, t, g, unknown, or other
  misc_difference 62: note = a, c, t, g, unknown, or other
  misc_difference 63: note = a, c, t, g, unknown, or other
  misc_difference 64: note = a, c, t, g, unknown, or other
  misc_difference 90: note = a, c, t, g, unknown, or other
  misc_difference 91: note = a, c, t, g, unknown, or other
  misc_difference 92: note = a, c, t, g, unknown, or other
  misc_difference 93: note = a, c, t, g, unknown, or other
  misc_difference 94: note = a, c, t, g, unknown, or other
  misc_difference 95: note = a, c, t, g, unknown, or other
  misc_difference 96: note = a, c, t, g, unknown, or other
  misc_difference 117: note = a, c, t, g, unknown, or other
  misc_difference 118: note = a, c, t, g, unknown, or other
  misc_difference 119: note = a, c, t, g, unknown, or other
  misc_difference 120: note = a, c, t, g, unknown, or other
  misc_difference 121: note = a, c, t, g, unknown, or other
  misc_difference 122: note = a, c, t, g, unknown, or other
  misc_difference 123: note = a, c, t, g, unknown, or other
  misc_difference 149: note = a, c, t, g, unknown, or other
  misc_difference 150: note = a, c, t, g, unknown, or other
  misc_difference 151: note = a, c, t, g, unknown, or other
  misc_difference 152: note = a, c, t, g, unknown, or other
  misc_difference 153: note = a, c, t, g, unknown, or other
  misc_difference 154: note = a, c, t, g, unknown, or other
  misc_difference 155: note = a, c, t, g, unknown, or other
  misc_difference 176: note = a, c, t, g, unknown, or other
  misc_difference 177: note = a, c, t, g, unknown, or other
  misc_difference 178: note = a, c, t, g, unknown, or other
  misc_difference 179: note = a, c, t, g, unknown, or other
  misc_difference 180: note = a, c, t, g, unknown, or other
  misc_difference 181: note = a, c, t, g, unknown, or other
  misc_difference 182: note = a, c, t, g, unknown, or other
  misc_difference 208: note = a, c, t, g, unknown, or other
  misc_difference 209: note = a, c, t, g, unknown, or other
  misc_difference 210: note = a, c, t, g, unknown, or other
  misc_difference 211: note = a, c, t, g, unknown, or other
  misc_difference 212: note = a, c, t, g, unknown, or other
  misc_difference 213: note = a, c, t, g, unknown, or other
  misc_difference 214: note = a, c, t, g, unknown, or other
  misc_difference 235: note = a, c, t, g, unknown, or other
  misc_difference 236: note = a, c, t, g, unknown, or other
  misc_difference 237: note = a, c, t, g, unknown, or other
  misc_difference 238: note = a, c, t, g, unknown, or other
  misc_difference 239: note = a, c, t, g, unknown, or other
  misc_difference 240: note = a, c, t, g, unknown, or other
  misc_difference 241: note = a, c, t, g, unknown, or other
  misc_difference 267: note = a, c, t, g, unknown, or other
  misc_difference 268: note = a, c, t, g, unknown, or other
  misc_difference 269: note = a, c, t, g, unknown, or other
  misc_difference 270: note = a, c, t, g, unknown, or other
  misc_difference 271: note = a, c, t, g, unknown, or other
  misc_difference 272: note = a, c, t, g, unknown, or other
  misc_difference 273: note = a, c, t, g, unknown, or other
  misc_difference 294: note = a, c, t, g, unknown, or other
  misc_difference 295: note = a, c, t, g, unknown, or other
  misc_difference 296: note = a, c, t, g, unknown, or other
  misc_difference 297: note = a, c, t, g, unknown, or other
  misc_difference 298: note = a, c, t, g, unknown, or other
  misc_difference 299: note = a, c, t, g, unknown, or other
  misc_difference 300: note = a, c, t, g, unknown, or other
ggtggtaatgccatgtaatatgccttcgaannnnnnngcaggcatacactgaagttcnnnnnnnctacaaactcttcctgttagttagcnnnnnnnaacttgacacttaatgcttgnnnnnnntgcaaggggtgttatgagccatcctnnnnnnnccagcagtttggccagccacnnnnnnnttgacctttgttactactctagccannnnnnntattaaatactcgtattgctnnnnnnncccagtcacgacgttgtaaaacggannnnnnncacagcttgtctgtaagcggnnnnnnngtactagcctgtgtgaaattgttatccgct

SEQ ID NO: 34 - RNA, 40 nt, synthetic construct
  source 1..40: mol_type = other RNA
agatttctacttttgtagattattaaatactcgtattgct

SEQ ID NO: 35 - DNA, 12 bp, synthetic construct
  source 1..12: mol_type = other DNA
  modified_base 1: mod_base = OTHER; note = 5' 6-Fluorescein thymine
  modified_base 12: mod_base = OTHER; note = 3' Iowa Black FQ thymine
tttttttttttt

SEQ ID NO: 36 - DNA, 19 bp, synthetic construct
  source 1..19: mol_type = other DNA
acatgaggatcacccatgt

SEQ ID NO: 37 - DNA, 25 bp, synthetic construct
  source 1..25: mol_type = other DNA
ggagcagacgatatggcgtcgctcc

SEQ ID NO: 38 - DNA, 15 bp, synthetic construct
  source 1..15: mol_type = other DNA
gccctgaagaagggc

SEQ ID NO: 39 - RNA, 30 nt, synthetic construct
  source 1..30: mol_type = other RNA
tattaaatactcgtattgctgttcgattat

SEQ ID NO: 40 - RNA, 25 nt, synthetic construct
  source 1..25: mol_type = other RNA
tattaaatactcgtattgctgttcg

SEQ ID NO: 41 - RNA, 22 nt, synthetic construct
  source 1..22: mol_type = other RNA
tattaaatactcgtattgctgt

SEQ ID NO: 42 - RNA, 21 nt, synthetic construct
  source 1..21: mol_type = other RNA
tattaaatactcgtattgctg

SEQ ID NO: 43 - RNA, 20 nt, synthetic construct
  source 1..20: mol_type = other RNA
tattaaatactcgtattgct

SEQ ID NO: 44 - RNA, 19 nt, synthetic construct
  source 1..19: mol_type = other RNA
tattaaatactcgtattgc

SEQ ID NO: 45 - RNA, 18 nt, synthetic construct
  source 1..18: mol_type = other RNA
tattaaatactcgtattg

SEQ ID NO: 46 - RNA, 17 nt, synthetic construct
  source 1..17: mol_type = other RNA
tattaaatactcgtatt

SEQ ID NO: 47 - RNA, 16 nt, synthetic construct
  source 1..16: mol_type = other RNA
tattaaatactcgtat

SEQ ID NO: 48 - RNA, 30 nt, synthetic construct
  source 1..30: mol_type = other RNA
cacagcttgtctgtaagcggatgccatatg

SEQ ID NO: 49 - RNA, 25 nt, synthetic construct
  source 1..25: mol_type = other RNA
cacagcttgtctgtaagcggatgcc

SEQ ID NO: 50 - RNA, 22 nt, synthetic construct
  source 1..22: mol_type = other RNA
cacagcttgtctgtaagcggat

SEQ ID NO: 51 - RNA, 21 nt, synthetic construct
  source 1..21: mol_type = other RNA
cacagcttgtctgtaagcgga

SEQ ID NO: 52 - RNA, 20 nt, synthetic construct
  source 1..20: mol_type = other RNA
cacagcttgtctgtaagcgg

SEQ ID NO: 53 - RNA, 19 nt, synthetic construct
  source 1..19: mol_type = other RNA
cacagcttgtctgtaagcg

SEQ ID NO: 54 - RNA, 18 nt, synthetic construct
  source 1..18: mol_type = other RNA
cacagcttgtctgtaagc

SEQ ID NO: 55 - RNA, 17 nt, synthetic construct
  source 1..17: mol_type = other RNA
cacagcttgtctgtaag

SEQ ID NO: 56 - RNA, 16 nt, synthetic construct
  source 1..16: mol_type = other RNA
cacagcttgtctgtaa

SEQ ID NO: 57 - RNA, 50 nt, synthetic construct
  source 1..50: mol_type = other RNA
agatttctacttttgtagattattaaatactcgtattgctgttcgattat

SEQ ID NO: 58 - RNA, 45 nt, synthetic construct
  source 1..45: mol_type = other RNA
agatttctacttttgtagattattaaatactcgtattgctgttcg

SEQ ID NO: 59 - RNA, 42 nt, synthetic construct
  source 1..42: mol_type = other RNA
agatttctacttttgtagattattaaatactcgtattgctgt

SEQ ID NO: 60 - RNA, 41 nt, synthetic construct
  source 1..41: mol_type = other RNA
agatttctacttttgtagattattaaatactcgtattgctg

SEQ ID NO: 61 - RNA, 39 nt, synthetic construct
  source 1..39: mol_type = other RNA
agatttctacttttgtagattattaaatactcgtattgc

SEQ ID NO: 62 - RNA, 38 nt, synthetic construct
  source 1..38: mol_type = other RNA
agatttctacttttgtagattattaaatactcgtattg

SEQ ID NO: 63 - RNA, 37 nt, synthetic construct
  source 1..37: mol_type = other RNA
agatttctacttttgtagattattaaatactcgtatt

SEQ ID NO: 64 - RNA, 36 nt, synthetic construct
  source 1..36: mol_type = other RNA
agatttctacttttgtagattattaaatactcgtat

SEQ ID NO: 65 - RNA, 50 nt, synthetic construct
  source 1..50: mol_type = other RNA
agatttctacttttgtagatcacagcttgtctgtaagcggatgccatatg

SEQ ID NO: 66 - RNA, 45 nt, synthetic construct
  source 1..45: mol_type = other RNA
agatttctacttttgtagatcacagcttgtctgtaagcggatgcc

SEQ ID NO: 67 - RNA, 42 nt, synthetic construct
  source 1..42: mol_type = other RNA
agatttctacttttgtagatcacagcttgtctgtaagcggat

SEQ ID NO: 68 - RNA, 41 nt, synthetic construct
  source 1..41: mol_type = other RNA
agatttctacttttgtagatcacagcttgtctgtaagcgga

SEQ ID NO: 69 - RNA, 40 nt, synthetic construct
  source 1..40: mol_type = other RNA
agatttctacttttgtagatcacagcttgtctgtaagcgg

SEQ ID NO: 70 - RNA, 39 nt, synthetic construct
  source 1..39: mol_type = other RNA
agatttctacttttgtagatcacagcttgtctgtaagcg

SEQ ID NO: 71 - RNA, 38 nt, synthetic construct
  source 1..38: mol_type = other RNA
agatttctacttttgtagatcacagcttgtctgtaagc

SEQ ID NO: 72 - RNA, 37 nt, synthetic construct
  source 1..37: mol_type = other RNA
agatttctacttttgtagatcacagcttgtctgtaag

SEQ ID NO: 73 - RNA, 36 nt, synthetic construct
  source 1..36: mol_type = other RNA
agatttctacttttgtagatcacagcttgtctgtaa

SEQ ID NO: 74 - RNA, 20 nt, synthetic construct
  source 1..20: mol_type = other RNA
agatttctacttttgtagat

SEQ ID NO: 75 - RNA, 21 nt, synthetic construct
  source 1..21: mol_type = other RNA
agatttctactatttgtagat

SEQ ID NO: 76 - RNA, 20 nt, synthetic construct
  source 1..20: mol_type = other RNA
agatttctactgttgtagat

SEQ ID NO: 77 - RNA, 20 nt, synthetic construct
  source 1..20: mol_type = other RNA
agatttctactttcgtagat

SEQ ID NO: 78 - RNA, 21 nt, synthetic construct
  source 1..21: mol_type = other RNA
taatttctactaagtgtagat

SEQ ID NO: 79 - RNA, 20 nt, synthetic construct
  source 1..20: mol_type = other RNA
gaatttctactattgtagat

SEQ ID NO: 80 - RNA, 41 nt, synthetic construct
  source 1..41: mol_type = other RNA
agatttctactatttgtagattattaaatactcgtattgct

SEQ ID NO: 81 - RNA, 40 nt, synthetic construct
  source 1..40: mol_type = other RNA
agatttctactgttgtagattattaaatactcgtattgct

SEQ ID NO: 82 - RNA, 40 nt, synthetic construct
  source 1..40: mol_type = other RNA
agatttctactttcgtagattattaaatactcgtattgct

SEQ ID NO: 83 - RNA, 41 nt, synthetic construct
  source 1..41: mol_type = other RNA
taatttctactaagtgtagattattaaatactcgtattgct

SEQ ID NO: 84 - DNA, 20 bp, synthetic construct
  source 1..20: mol_type = other DNA
  modified_base 1: mod_base = OTHER; note = 5' 6-Fluorescein cytosine
  modified_base 20: mod_base = OTHER; note = 3' Black Hole Quencher-1 cytosine
caagcttcccgttctcagcc

SEQ ID NO: 85 - DNA, 10 bp, synthetic construct
  source 1..10: mol_type = other DNA
  modified_base 1: mod_base = OTHER; note = 5' 6-Fluorescein adenine
  modified_base 10: mod_base = OTHER; note = 3' Iowa Black FQ adenine
attattatta

SEQ ID NO: 86 - skipped

SEQ ID NO: 87 - DNA, 12 bp, synthetic construct
  source 1..12: mol_type = other DNA
  modified_base 1: mod_base = OTHER; note = 5' 6-Fluorescein cytosine
  modified_base 12: mod_base = OTHER; note = 3' Iowa Black FQ cytosine
cccccccccccc

SEQ ID NO: 88 - DNA, 12 bp, synthetic construct
  source 1..12: mol_type = other DNA
  modified_base 1: mod_base = OTHER; note = 5' 6-Fluorescein adenine
  modified_base 12: mod_base = OTHER; note = 3' Iowa Black FQ adenine
aaaaaaaaaaaa

SEQ ID NO: 89-90 - skipped (2 entries)

SEQ ID NO: 91 - DNA, 12 bp, synthetic construct
  source 1..12: mol_type = other DNA
  misc_difference 1: note = 5' 6-Fluorescein adenine, cytosine or thymine
  misc_difference 12: note = 3' Iowa Black FQ adenine, cytosine or thymine
hhhhhhhhhhhh

SEQ ID NO: 92 - skipped

SEQ ID NO: 93 - protein, 1185 aa, synthetic construct
MPQSNSQQKKPSYNGRTGYALHTARLSYVGKEGMRSAKIPLDGEDSTRNTLWDNVRADYIGQVGEMNIGDWYEGEQKGAHVYALADFWLDSLRAGVIFSHKEKEMHGVIAHLNGDETFSKKFSLALAERNPELAALADNTALYQFFISGKRPGKKTDEEFFWSVRNSVFPRFRNVEDATVDAYARRLVDTVLKQTKDDTEADTLKIVLAKEYVGFDPTPDIPYKHTCFFHPQTSLLPDASAKQILAMYKELATQKDPTLYGYDGENESDVMKQIFGLFNNQGAFANYFNTTLADFRAGGEHTEQAIVTVSPYWATHRNELHARIMFLVERARMLPEPHLTKGWHDYRSTFGGKIESWVSNVLRQEKEIAKQLFGGIEESGKGARKKETFVRGHKQILEEVLADTCLSDDTKAIAHECFDLVLRMEISGITDADLLLYRTMLGDARIGMNVDYQTAYPELKGKTEKERKQEEKEKRAYRKYATLYTNIKLIPNFLGDSKRIVYKKFIQSAEILHTSIATLASLEMTLSEAPISPKFTSDDEAEIFVTKLYETLRKKYAVLNATRFRGVIEGIFRHLKGTLNGEMFDMMNVLERADETESTQKIEYVCYTPPYARVHPRRRTVDIIIKNEDIPALLDTLLVALKPRWDDIMAGGDIGELADAVEMEKIRMGIVVALWYRHPVARILDELSEELFSSAHAFAKLKGNPETFSGEDLGRLLQSGILSEMKGAITTMSRVEYTERYTGQLMNTETHMPLCRTEDGWHILSGKYQKKEKDGMIAVPIKKGKGFEGETDYTTEYIHTQDLLHLNTSVYQLQFLDKTLPQGKRTISWWDAHDIAFSLNSYSFIAERNMRIDWDISQKKFRITPGEEKRLFVSIPFSFSPKNKATDTLEKRTRYMGIDVGEYGLAWTIIETDAVHKKVVRILKQGFIYEPLTHTVRTYVATLKNLQTKGTFGMPNTKLERLRENAVGSLRNKVHDIAMRYDAIPVYEFEISNFETGSKRTTVIYNSVKRADVGRGGSEADKAEASLVWGKTGKNFGKEIGAYATSYICSRCGYSPYEASVIEGTRATLDMEKKLSRPSLNDFLKDHSAYKTREEFAMRGNKESIWKERRGNSAIYVCQHCGHVTDADVQASYWIAVKSFAKSLRSKKEEKDNSPVSFEEMSAIHDISSGTTADNKRITRLRIDI

SEQ ID NO: 94 - protein, 990 aa, synthetic construct
LDPAVIATIDDLIETFFTSNGDTIERDKQNQYWRDQWGLDKSLYDLDGDKDLTFYPLPKLEMTPDAGAHDCLRKYRNWIDDNQDTFGLDDQEAKEAFLRLCWGNLQASGNYNAFSNYFNQIVNTLQSEDGTDDVVDDLCTASDVWSGQENHLRERIAFLADGARRLGQPKCVADWSKYRSTLGGRLASWLSNTIRQEEFLAETTQKQVEELTDIATRLDNEFYAEAFSAADDTRDRQLASEVETAQDTLRKLRDNEGFNPDALSVYRDSIGRLRSLLNRAHQILGDDTVDAYEAYPALHEQVRLVPKFVGGAKEARFSKYRNSLHMLKRGVTFLQEEAPDAPDAAGVRNSDEEFESYFLRQLNALQRFYQDANSQRFRDIVSDLFARFDIDISAVSSAKTFYISPYSNRDNRQVINVDTDNKDVSNLLARWANEIKPYWDDILATENWEEIMDAVKIDRIRYGWIGRLSADTTIHIADDLSDHFPEAETYRDLHGDTLSGTQADNFLQRVILSEMEGRVNEASREEFTVRYVVQPQDAGKKSNYPLVVSSNGGDKITAGTRMQWYIKDNDTSFAHSETNRSVWTLSDSKTFANGLEKKYVKPQTNLFAIKTSKYQLQFLDNALPGGAKRFRDDGGFDLELQDHSFIAEETLSVNWNFDTGNLHLESTDKNMYAAIPVAVIPENEDSDRDNRTRYLGIDVGEYGLAYTIIETDPEVSILKTGFEHEPTLRNIRKQVTNIKSKQRTGTFGVPSTKLARLRKQAVTKLRNRMHDLAVRFDAIPVYEWQISNFETGSARVTNVYNSVKRADTSSGMGSDAESGEAELVWGDSSWRVGREVSAFATSNMCSNCFESVYAYPDEAFSLQSVESEYAYYTVDGTQENVMAFLSDTDPEGISGNKLKQAVKDFSRPSIDTLVERQTNDFDLSKGKTTELIRQRGNSALFECPFCHAVNDADIQASLFIGLRAILKDRTGEDEEITIPGLLTFAESADV

SEQ ID NO: 95 - protein, 1192 aa, synthetic construct
MNLFMTEDTNRKKYKGITGYSLHNKRLEYTGKSSIRSIKYPLIPNNWQIYHSFCDDIINDYERINGAVNITTYAKNPKIYSLFDFWIDSLRTGVIFRESANSLKNSLEKLRSKKSTAKISFDLMKVPAKKKLDSELFINKFVLSTGVRSTSTKQSLENKLLGCFKEKYRTNADVKKIAKKISSDLLPNRKALSQTEQKKFWHENYNLQIPAKKEKSSRNLTFYILPELELDKNLSVLGCIKRLKSIFDKENAKYLGLTSNFNAFSNYLNEVFSLFYDNKLKRISAALKEISKDWEGREDGLIKRLEFLSQKSRLLGKPRLTKSWADYRSVWGGKLQSWYSGFDRQASLIRKAIKDNESELTKLKKEVVKPRSSKKKRYLFQKKNLTRLIDHLLKLSPKIKSKNNLEFAQIDLYRDLLSQLRINLNEFYQKFLQTKKDKKEGKAVDKVYKSLFKNLRLVPDFFGESKKKNYEKYIFKTIPIIKQGIAITHTIEGQLLRGVDFSLVTQDYFYRQLEKINCKLRSNALNSSQKIHQFQNIIKTYNKGILSKKDEVFYKSQYSNNKKLKLIAWKNLNYGEELKKIYFKLKEEYSLNKSPDKLLDYIEILKIRIGWLLFLSSIRINTSEFDFSLFEKAKTYSQNFGSILKGEDRSFFIMSLILSEIRGAASLFSRKFFTARYVAQIINSEKKFPLIYIPKKKNDKNITLPSRINRMPDSNKKLLNKIDTSLMFFYPHQWGVVIPRNNKEITNGKESIKTIDFTGKNSQGLKVDEHSFEQIFNIYSSKYQIHFLDRLLYKTKKWKKISLALSEPFLIVEEEVKVKWNLVTSKPELLTDKNKKLFISIPFNLNPETKWRNQNKIIEKRNNYIGIDVGEYGLAWAVVSFREKNPKIIDKGFVFDPVVRKIADRIQDIKESQTKGTFGVPSTALARVRENAIKSLRNKIHYLALKYNAKIIFEYQINNFETGSNRVSKVYRSVKVSDVKLENKADELVKNQVWGNYSKYIGNHISAYATSYTCTKCYRSIYDECSKDKRYLIVKKWKNILDIKINKSLIAKGYTKKQQINTGDKLSYKVTMNALKGYARPPANSEALSEINIQEKDLQKFVTKRGNCAIFICPFCGHVADADIQAALNIACRGYLRDNKTKKFDYLEAMKIYMKNVKFPIVSLEYIQPTLIDKKIRLTEKPKQKELRIKHS

SEQ ID NO: 96 - protein, 1198 aa, synthetic construct
VKKKYKKHKGITGYTLHNKRLVYSGNSSIRSIKLPLKFNGNPQILQDYYDSLIYDYEKMNGPVNISTFFKDPSLYSLFNFWLDCLRVGVIFASSTASLQDTVYQVFGVLSPTQKAIKAIPEHIRKELNEKNFIEKVLLSEGMRSRTEKDSFINKMKKCFDIPQNTPEQELPGEKLIILETIQKIAEKMFVNGQVVDYKTQKDIWKEELNVEFPQKPTSKSTFYIIPKLLDRSKYSDLVDLEHITPIDLINNFKSFVSETWGLDEENKAVTDKTKSLVGLDSKFNGFSNFLNFVFKLLLDADIEQIKQKLNEIFDLSHAWNDNITDLDERLIYLSQKAKKLGKPKFRNGWHEYRSVFGGKLSSWFSNLLNQEKEIREQLIKHREEIEEVLKFVINNDSSSSIVTTLDSLKGLSETILNSENILEKEKEINLYREILSEFKFNFNYFYQKNSDKKNSEKDVKKAYPNLFKNLRVIPNFFGGSKYEQYDKYVYKTFKIINEGTQITLDLEKILLKNLETNNENIQIDKEYFQKQLQTLLNKIKTDALEYKEIKNIVELILKKYNNGNLPKEFEAFYVSPYSRDKRLKQINLPDELDFTQELLNVHKKLSSFNVGEFNADIVKFLSFVEILKIRIGWVLNLTKNNDLDVSRYDFTQFEKAKTYVDNFGTILKNDQRNIFINNFILSEIRGALSKASRKEFTERFVIQYVDSEKKNLLVYVPNIANNKNIITPHGVTIPKNMNELLSEKNKETKNFRRSLFFYPHRWKVSLFELKKVKNSNFNALSLQKKNFTNLVPVNIDDDNTFDICTSKYQIHFLDRLLYQTKKWKRMHIEFSSPALILEVKNNVKWDLETQRPHLSIKQQVLYINYPINLKAEESKNHIQTQAKNYLGIDTGEYGLAWVALDFSDLSAPKLLDKGFIYDDSLRKIRDRVDEIKDTQIKGTFGIPSTKLARIRKHTITTLRNKVHDLLLKYNAKVIYEYSISNFETGSQRVTKIYRSIKVSDVFAETDADKSVKKLVWGKENFNTGNHISAYATSYTCSKCCRSIYFEQEKQNENVVVEKDNNLLKVKLREGVFVWAYSSDKSISEGSVVKNEDLIKMVREYARPPENSEALKRVNPKINDIFEKRGSSVIFICPYDDCNHLSDADIQAAQNIAIKGFLSDLRNSPKRNSAKINDEGKLQEFIEYIQNPKNKVPIIELIE

SEQ ID NO: 97 - protein, 1230 aa, synthetic construct
SRPSDVTGIMANRQISPVDSTNNFIFTLYVSFDTVLYARILAFLTLSCLLVGFLEYIYPLGVIYLIMPTNQQYDGLTGYQLHTERLRHTGKSGIRTFKLPLKTNDQELFGNFFENIKHDHEGQIGATNITTWTEEQKSSRQIYSLLDFWLDSVRAGVVFASSSAELNELLSVCGDEDAVDEQVKTAMRAINPTFFKHLKFNDFKEEIALKEGMRSSSTKNSVTRRLYKCLDCTEEDAPEEIQQAVADLIKTFFTTDGKIISRNDQDDYWQVHFGLDKSLYKLDSDIKLTFSFLPDIPFTPEADAHMCLDKYRCWIDENAEKFQLESKEDKTNFLQIHWGIEGNYNAFSNYFNQIIDLLREEEGRQKLLDALLNTSDLWEGAETELKKRFDFLVDKAKQLPKAKCVDSWSDYRTTFGGRLSSWLSNTLNQEEFIKDTVSDQKEELKQIIKNSEINLYKKAFSAADDNRAVELSHEAVIAQKTLEKLQKSSSFEPQLLGVYRDNLGRLRSLLNKAHQLLPDEIENKSAHEVYSALHERIRLMPKFIGGAKAARFEKYVKSLQILKQGASFLENFEEKVKEAMKSSVSVEVEEISEGYFLRQLNTLKRFYDNANDSRFKGKLSSLFDKDLGVNIEAVSNRETFYISPYSKHDNRAVISVEVANYQQLLKSWVNELKPYWGNIIATENWGEIIDAMQLERIRIGWIYKLYPKLTFAISDDLDELFAKAATYRDLHGHEVEGSLAANFLQRIILSEIEGRVKEASREKFTVRYVIQPVGSEKTTKYPLFVTSNKDKLSATERKQWYIRDINSSFSRSETDKKGWILKNGKDFSNGFQEKYISDNTDLFKIISSKYQLQFLDKALPNGAKKFGKDGDVNLKLSEHSFIAEETLQASWDFSTDTVSLKSVDKDLFVAIPVNIEPESQRSSREERTRYLGIDVGEYGLAYTLIETIPKVRVIKSGFLYEKTLRNIRKQVTNIKNSQRLGTFGVPSTKLARLRTQAITKLRNKVHDLVVRYDAIPVYEWQISNFETKSGRVTKIYNSVKRADTNSGANENADGDEAALVWGKRPYKIGQEVSARATSNMCSNCFKSIYQFTEQDFTEESRSGEYVEFKIAKNTVKGFVPNDEKITSKQKMINAVHDYARPSVDILVKRNDVGVDKEILVNFLEHRGASALYECPFCSHLSDADVQASLFIGLRAICRDQDDETTKVKISDLMDFAQKQNIDPISADFIQ

SEQ ID NO: 98 - protein, 1193 aa, synthetic construct
LKRIAKFRHDKPVKREAWSKGYRVHKNRIINKVTRSIKYPLVVKDEWKKRLIDDAAHDYRWLVGPINYSDWCRDPNQYSILEFWIDFLCVGGVFQSSHSNICRLAIQLSGGSVFEQEWKDLSPFVRANLIQGIKPAEFIGFLTAEFRSSSNPKNFISKFFEGSNEDLESLTNEFASIVDFIKAKDISLLRKSLPSCKKIAPNLWEKAVGSHSTNELLKLLTKYTRVMLVAEPSHSDRVFSQTVLQSNDQDDPELTGPLPSHKVGKASYLFIPEFIREVNLDKISKLDLSAKSKLAVEQVKKLSELTSDFKQIENQSEAYFGLSTSFNELSNFLGILIRTLRNAPEAILKDQIALCAPLDKDILKITLDWLCDRAQALPENPRFETNWAEYRSYLGGKIKSWFSNYENFFEIPQAASSQQNNNREKKLGNRSAIRALNLKKEAFEKARETFKGDKGTLEKIDLAYRLLGSISPEVLQCDEGLKLYQQFNDELLVLNETINQKFQDAKRDIKAKKEKESFEKLQRNLSSPLPRIPEFFGERAKKGYQKARVSPKLARHLLECLNDWLARFAKVEESAFSEKEFQRILDWLRTSDFLPVFIRKSKDPPSWLRYIARVATGKYYFWVSEYSRKRVQIIDKPIAQNPLKELISWFLLNKDAFSRDNELFKGLSSKMVTLARIMAGILRDRGEGLKELQAMTSKLDNIGLLHPSFSVPVTDSLKDAAFYRAFFSELEGLLNIGRSRLIIERITLQSQQSKNKKTRRPLMPEPFINEDKEVFLAFPKFETKNKVKGTRVVYNSPDEVNWLLSPIRSSKGQLSFMFRCLSEDAKIMTTSGGCSYIVEFKKLLEAQEEVLSIHDCDIIPRAFVSIPFTLERESEETKPDWKPNRFMGVDIGEYAVAYCVIEKGTDSIEILDCGIVRNGAHRVLKEKVDRLKRRQRSMTFGAMDTSIAAARESLVGNYRNRLHAIALKHGAKLVYEYEVSAFESGGNRIKKVYETLKKSDCTGETEADKNARKHIWGETNAVGDQIGAGWTSQTCAKCGRSFGADLKAGNFGVAVPVPEKVEDSKGHYAYHEFPFEDGLKVRGFLKPNKIISDQKELAKAVHAYMRPPLVALGKRKLPKNARYRRGNSSLFRCPFSDCGFTADADIQAAYNIAVKQLYKPKKGYPKERKWQDFVILKPKEPSKLFDKQFYRPN

SEQ ID NO: 99 - protein, 1189 aa, synthetic construct
MLPFGAGALIIILNMQKGYNLHKKRIDLLNGEILRTIKYPLKPLNENSEKNKKQFELQIINDDLKIRGDLNLNDYLNYTFKNNHIYNLFDFWIDSLKCGVIWQPSFNNLKNLIEKIYLNKTISEEIFDNSIPEIKKFFDKNKFIENFILRTGIRENKSKKSFEKDLIKSLNDEFNKEEINKEAQDLIDKIINTFFNENKLKKLTIEEQNNLWLNKFNIDKSKLKTKELKYNYTFFIIPELIKFKENNLENLLNIRYEFIKNKNLNIEILQEILGLENNFNSFSNYFNEIIKNLQNDEYNLIYEDFISFYKFTNEEKEKILQSLKYLSEKSKQLGSPQILNLSWADYRSILGGKIQSWFTNYLQRKEKLEEQLNSLKENINKTKEYLLENFQENTDLKEIIYFLDKLFELSEVSKINNLFFDIHSTCKLKINNFFQKNIKQNEDDTVESNENLKGIFEKIYKPLNFYGQSRRENNIKIINSLQTVELGIIYTKKILNFLKNDLDFNKEENFEEIYRKNLNFLWNKLKENSLNSKNFKSKYKEIIIKNLNSYSNIKFEKIIKRPSYYVFYKSIYSKGTNLQIEFNKEINFKNIFIENINELYNFLINLEDKEIITDKNLLLDWIELSKNISSYFIKFTNIEKINLKEIFKDKDLNFFSNIAKYFETFGNVVEKQGLSYFVQSIFFSEFKGAATLYTKNKYLAKYNIQIIHSNEKFPLYYYSNNLKNHNKLLNPKDLIREKHNYFVSLKKIKILKNKDLEFLLLNKDNFQKIYNSFEDNKNDLLKLESSFYQIQFLEKFIYKPKNWENSKIELSEWSFILEKEYEIKWDLENKIPILNEIQNSKKNKLYIAIPFKISSYQKEYEEIKKSKENYILGIDVGEYGIGWVLINYKNKELKIINKGFFVSKNIAKIKDEFSKIQKKSQKGIFNESSNLISEIRKNAIGELRNKIHSVFIFYHSKNICYEYSINAFETGSGKTTAIYKSIKKSDTLSIIDADKKEQMHIWGKKLNKEIEIGKNLSSYASSYICSKCGKSLYQLKETDLENNEIKIIEREGNILKIKTPYGNVYAYSLKKEHKVDFIFNKTKSSFKELQKIIKDFARPPLNNNSEVIRKFTNLKENEIDILHQKRGSSCVFVCPFCLHVSDADIQAALMMSIRGVINNETKSKGEKIDYFQKNLEFLNELKEKPVIDL

SEQ ID NO: 100 - protein, 1276 aa, synthetic construct
MGQRGERCVRRKKSFKGYRLHAERLALASEASNLETDTIRTIKFPLVGTDPLVKDAFAAVRHDYLAIHGPLTFHDWVGSQGRESYSLVDFWIDAVAIGGVFGASASKLRQLAAELAGEAPVEAFKARYAAAVNPALEHLFDWAKIAEFFSKDLRGRAEGCFRKGFRDEPAAREVLSRYNQARKELRQSESYRSSEDKGALLWECRARALFGFSAPQDQFPSRPGGATEDSLGAEMPDLKLTFLLVPSCIQESVGGGKCSAPWRIDRTEPSFSLQQGRTSAVLQLLGFVEGLKGGAGLSEEKDDLLGLTDNYNALQNFFNVFLQELREDPSSVARAMVCLYPAKLHDSIRDEIVSKCEALSRLAQRLAQPKLAANWSEYRSFLGGEILSWVSNQKRLIGELTTGLQSKVEALEEVKGRLAEQRFTGALSQPKWQLLPSQIAEVIQVLHGLQASLGAGGVVPRDFARTFDLFQDLLASLNADLNRFDQLHGLFNRASRQRAGRPGPRAARSTAQDDAEQTDSLQSKLSKRVQQVPAFFGETKKAHYPKFRAAPALISWFVDTLKTAFAAAQVTKRPLTPEEVERMLRWLADTAEYGSLEEKILKAETCPLRHLLQLRRFPRSSRPYCYKSRYVRQRREPALSAEQFSAWMGTSPELSADGVVAWFADEVNRTLSGVKGGRNELLPLFRALIKLWPGVIRLGGNPSADVRERLLPRLVEFFGSAKHGEMRKESFPRLSAYLDFVGALSGECSLEDAQADRILSKLLAECSGLLALASYSSFIARYSLQHQKGAQTNLLVQGAEDPRTARFSMELVGLRPASGGVAHANAHAPDLRLLMKAQVFNRNTSPFRGGGAVLSIQSSKYQVQFLRRALKEAQAGAWAVAPSGSFLIAETDVRVRPNWTRGEMEVITDEAALPRLYSSIPFNLSVKRDPAPEPEMFVRPGGANDERGRVIGVDIGEYALTFSVVEWSRAGERVQFRVLDTHERVLKDGAHRVLKDEVRRLKKRQVRGTFSTRSSAVADLRETLIGAYRAKLHDLVVRYRAPLVFEFNVSAFEAGGNKIKKIYDSVKISDTGGDSDADKNVRKHYWGKKFQKGFVTGREIGAKHTSQLCSSCRRVALEELDELLRSESFEDKKELDAHGRYLWHEVNVGGTKFRAFLGRDESLSKPESVKRFVRAALRPPLKRFQRKLPDAARKRRGNSALFWCPFIDCNHLSDCDVQASVVIAVKAVLNQLFKEEYKKGWLKLVQDRARVELPVVALEARYFRNPYAPREAEGLN

SEQ ID NO: 101 - protein, 1160 aa, synthetic construct
MTKDNSQHNGLTGYRLHTKRLGHTGNAGIRTFKLPIHAEDNSRHDSFFADIKNDHEKQLGATNITSWLESREGDADMYSLLDFWLDSLRAGVVFAPKSRALNKLLADIAGSSIDEQVKASMREKQPAFVQAVNFADFKEKFALKGAIRSTSTKDSVRRRFLQCFDEDEDDLDPAVIATIDDLIETFFTSNGDTIERDKQNQYWRDQWGLDKSLYDLDGDKDLTFYPLPKLEMTPDAGAHDCLRKYRNWIDDNQDTFGLDDQEAKEAFLRLCWGNLQASGNYNAFSNYFNQIVNTLQSEDGIDDVVDDLCTASDVWSGQENHLRERIAFLADGARRLGQPKCVADWSKYRSTLGGRLASWLSNTIRQEEFLAETTQKQVEELTDIATRLDNEFYAEAFSAADDTRDRQLASEVETAQDTLRKLRDNEGFDPDALSVYRDSIGHLRSLLNRAHQILGDDTVDAYEAYPALHEQVRLVPKFVGGAKEARFSKYRNSLHMLKRGVTFLQEEAPDAPDAAGVRNSDEEFESYFLRQLNALQRFYQDANSQRFRDIVSDLFARFDIDISAVSSAKTFYISPYSNRDNRQVINVDTDNKDVSKLLARWANEIKPYWDDILATENWEEIMDAVKIDRIRYGWIGRLSADTTIHIADDLSDHFPEAETYRDLHGDTLSGTQADNFLQRVILSEMEGRVNEASREEFTVRYVVQPQDAGKKSNYPLVVSSNGGDKITAGTRMQWYIKDNDTSFAHSETNRSVWTLSDSKTFANGLEKKYVKPQTNLFAIKTSKYQLQFLDNALPGGAKRFRDDGGFDLELQDHSFIAEETLSVNWNFDTGNLHLESTDKNMYAAIPIAIIPENEDSDRDNRTRYLGIDVGEYGLAYTIIETEPEVSILETGFEHEPTLRNIRKQVTNIKNKQRTGTFGVPSTKLARLRKQAVTKLRNRMHDLAVRFDAILVYEWQISNFETGSARVTNVYNSVKRADTSSGMGSDAESGEAELVWGDSSWRVGREVSAFATSNMCSNCFESVYAYPDEAFSLQSVESEYAYYTVDGTQENVMAFLPDTDPEGISGGKLKQAIKDFSRPSIDTLVERQTNDFDLSKGKTTELIRHRGNSALFECPFCHAVNDADIQASLFIGLRAILKDRTGEDEEITIPGLLTFAESADV

SEQ ID NO: 102 - protein, 1276 aa, synthetic construct
MGQRGERCVRRKKSFKGYRLHAERLALASEASNLETDTIRTIKFPLVGTDPLVKDAFAAVRHDYLAIHGPLTFHDWVGSQGQESYSVVDFWIDAVAIGGVFGSSASKLRQLAAGLAGEDPVEACKGRYVSGVGPALESTLDWAQVADFLGKDARANVKWCFKEEFREAPAAHDIIARYNQAKTELQQSEPYRSSEHKDVLLRECRERALFGFSVPQDPLPLVPGSVAEESLGLEAADLRLTFLLVPSFIQDSVEGGKCSAPWTVDSTDFSFSLNQGRTQAVLQVLGFAKRLKEGARLSQGEEELLGLTDNFNAFSNFFNLFLHKLRADPSSVARMMVSLYPARLHDSIRDEIVSKCEALSRLAQRLAQPKLATNWSEYRSFLGGEILSWVSNQKRLIDELATGLQSKVEALEEVKGRLAEQRFTGALSQPKWQLLPSQIAEVIQVLHGLQASLGAGGVVPRDFARTFDLFQDLLASLNADLNRFDQLHGLFNRASRQRAGRPGPRAARSTAQDDAEQTDSLQSKLSKRVQQVPAFFGETKKAHYPKFRAAPALISWFVDTLKTAFAAAQVTKRPLTPEEVERMLRWLADTAEYGSLEEKILKAETCPLRHLLQLRRFPRSSRPYCYKSSYARQRREPALSAEQFSAWMGTSPELSADGVVAWFADEVNRTLSGVKGGRNELLPLFRALIKLWPGVIRLGGNPSADVRERLLPRLVEFFGSAKHGEMRKESFPRLSAYLDFVGALSGECSLEDAQADRILSKLLAECSGLLALASYSSFIARYSLQHQKGAQTNLLVQGAEDPRTARFSMELVGLRPASGGVAHANAHAPDLRLLMKAQVFNRNTSPFRGGGAVLSIQSSKYQVQFLRRALKEAQAGAWAVAPSGSFLIAETDVRVRPNWTRGEMEVITDEAALPRLYSSIPFNLSVKRDPAPEPEMFVRPGGANDERGRVIGVDIGEYALTFSVVEWSRAGERVQFRVLDTHERVLKDGAHRVLKDEVRRLKKRQVRGTFSTRSSAVADLRETLIGAYRAKLHDLVVRYRAPLVFEFNVSAFEAGGNKIKKIYDSVKISDTGGDSDADKNVRKHYWGKKFQKGFVTGREIGAKHTSQLCSSCRRVALEELDELLRSESFEDKKELDAHGRYLWHEVNVGGTKFRAFLGRDESLSKPESVKRFVRAALRPPLKRFQRKLPDAARKRRGNSALFWCPFIDCNHLSDCDVQASVVIAVKAVLNQLFKEEYKKGWLKLVQDRARVELPVVALEARYFRNPYAPREAEGLN

SEQ ID NO: 103 - protein, 1218 aa, synthetic construct
MANRQISPVDSTNNFIFTLYVSFDTVLYARILAFLTLSCLLVGFLEYIYPLGVIYLIMPTNQQYDGLTGYQLHTERLRHTGKSGIRTFKLPLKTNDQELFGNFFENIKHDHEGQIGATNITTWTEEQKSSRQIYSLLDFWLDSVRAGVVFASSSAELNELLSVCGDEDAVDEQVKTAMRAINPTFFKHLKFNDFKEEIALKEGMRSSSTKNSVTRRLYKCLDCTEEDAPEEIQQAVADLIKTFFTTDGKIISRNDQDDYWQVHFGLDKSLYKLDSDIKLTFSFLPDIPFTPEADAHMCLDKYRCWIDENAEKFQLESKEDKTNFLQIHWGIEGNYNAFSNYFNQIIDLLREEEGRQKLLDALLNTSDLWEGAETELQKRFDFLVDKAKQLPKAKCVDSWSDYRTTFGGRLSSWLSNTLNQEEFIKETVSDQKKELKQIIEKNLYKKAFSAADDNRAVELSHEAVIAQKTLEKLQKSSSFEPQLLGVYRDNLGRLRSLLNKAHQLLPDEIENKSAHEVYSALHERIRLMPKFIGGAKAARFEKYVKSLQILKQGASFLENFEEKVKEAMKSSVSVEVEEISEGYFLRQLNTLKRFYDNANDSRFKGKLSSLFDKDLGVNIEAVSNRETFYISPYSKHDNRAVISVEVANYQQLLKSWVNELKPYWGNIIATENWGEIIDAMQLERIRIGWIYKLYPKLTFAISDDLDELFAKAATYRDLHGHEVEGSLAANFLQRIILSEIEGRVKEASREKFTVRYVIQPVGSEKTTKYPLFVTSNKDKLSATERKQWYIRDINSSFSRSETDKKGWILKNGKDFSNGFQEKYISDNTDLFKIISSKYQLQFLDKALPNGAKKFGKDGDVNLKLSEHSFIAEETLQASWDFSTDTVSLKSVDKDLFVAIPVNIEPESQRSSREERTRYLGIDVGEYGLAYTLIETIPKVRVIKSGFLYEKTLRNIRKQVTNIKNSQRLGTFGVPSTKLARLRTQAITKLRNKVHDLVVRYDAIPVYEWQISNFETKSGRVTKIYNSVKRADTNSGANENADGDEAALVWGKRPYKIGQEVSARATSNMCSNCFKSIYQFTEQDFTEESRSGEYVEFKIAKNTVKGFVPNDEKITSKQKMINAVHDYARPSVDILVKRNDVGVDKEILVNFLEHRGASALYECPFCSHLSDADVQASLFIGLRAICRDQDDETTKVKISDLMDFAQKQNIDPISADFIQ

SEQ ID NO: 104 - protein, 1310 aa, synthetic construct
MSVARSKIVDWRGRTGYALQSHRLLFAARVSRDKKIGTVRTIRFPVKVEDRRISSWLTGSAISEYNTLIGSPLNELLSATSNGAGRITTLLDFWLDALRMGAFFQPSARRLTDLVFIFAGRGKDSPAELIWRSVPARLRKVLRRKVFIDEFVLESEMRRSKEIKNLRLGLLNVFKEEAGSFPETERIANNLILFLKRSKSGKLREGEKREAWKKELEENFQLKQGILDQWPDTGNLSFCLHPALEPDFERKTERRELIEKRLQELVRLARGTRCEVRLSEQRKSAAKKRGERSEEKRRAKVEQGKQQQQRKAIDFLGLDVECNGLNNFFGEVFEKLRSERGLKQLKEQILVLPLWQGKEDELERRLRFLAASARSLPASKLTGKWSDYRGTLGGKIKSWWSNYMGFKLKLAKLLPAEEAASACLVKLLSSSREEEGDLEELRRNTLVCLRKNLGYLEEITASGGGAEALVKLYRKYGYLRGEIRRNLIRLIYQGKIFETEREKLIKKAGGGPISPRKCLPSQARKILDNLPRLPRFLGEAAGEEVKLMQEGPRALKIAYELALDADRQLREGSFCPPARDRYLHVLNSLLSLYHRLNSNILTATVERIIGEATRKKEVPPSGRLFYVSPKAPLHVRERLVDAPGISADGSKELAKQWERLITLTSPSSPERFLQHTTEQQMDEVELLKIRLAFLLHCYPKTSIAVERWRDLACRVPQTSASLLSALESPSESRLRREERSVVLNELGSVLRGIVTRKSRRAITLRSTIQLMGAEKKLQLIAAPVNPRDARWKRWERKFAREKSLSGQALSRFYRIPHRFGYYLKVGAGFRGRDGRGDNKREAGAQVKCLAFKEKGNEAELRSIPAARIAWIRTSREQLQFLEWLLVKPRRKRAILLKRREPMLVVERDLVLKWKREKLEAEAGTSPKLFINQPFQIIPQFEDENVLRLPYRRFLGVDIGEYGVGWCVLEEKGEKLRIVRRGFLYEPLVREVREYVAAWRESQVFGLFASPETKLARLRENAAHKIRNRLHDIALRYHAQPVYEYQVSAFEAGGDRVKRLYDSIKRADKIFDSRAPRALKSERVSVWGDNSKWGEEEVIRPGGAEVSARATSRTCTSCWRSVLDEGLVPREEKFRVEWLGQGVIRLNKEGSGRRQVSFFGFLSGKYKGVTEVIGQEARALAEKFSRPPLPEGKKGVPRLSVALRFAFRERRRRGFSVPSLEQLAQFYGKRGSRALFICPFCLHIADADQQAALLVALTRWYSLEYGGGRLSGKLGRKAQGAKYFDLLDLLRRIEGSSPLRKKIELLTVSFP

SEQ ID NO: 105 - protein, 1228 aa, synthetic construct
MANRQISPVDSTNNFIFTLYVSFDTVLYARILAFLTLSCLLVGFLEYIYPLGVIYLIMPTNQQYDGLTGYQLHTERLRHTGKSGIRTFKLPLKTNDQELFGNFFENIKHDHEGQIGATNITTWTEEQKSSRQIYSLLDFWLDSVRAGVVFASSSAELNELLSVCGDEDAVDEQVKTAMRAINPTFFKHLKFNDFKEEIALREGMRSSSTENSVTRRLYKCLDCTEEDAPEEIQQAVADLIKTFFTTDGKIISRNDQDDYWQVHFGLDKSLYKLDSDIKLTFSFLPDIPFTPEADAHMCLDKYRCWIDENAEKFQLESKEDKTNFLQIHWGIEGNYNAFSNYFNQIIDLLREEEGRQKLLDALLNTSDLWEGAETELQKRFDFLVDKAKQLPKAKCVDSWSDYRTTFGGRLSSWLSNTLNQEEFIKETVSDQKKELKQIIEKNLYKKAFSAADDNRAVELSHEAVIAQKTLEKLQKSSSFEPQLLGVYRDNLGRLRSLLNKAHQLLPDLNKAHQLLPDEIENKSAHEVYSALHERIRLMPKFIGGAKAARFEKYVKSLQILKQGASFLENFEEKVKEAMKSSVSVEVEEISEGYFLRQLNTLKRFYDNANDSRFKGKLSSLFDKDLGVNIEAVSNRETFYISPYSKHDNRAVISVEVANYQQLLKSWVNELKPYWGNIIATENWGEIIDAMQLERIRIGWIYKLYPKLTFAISDDLDELFAKAATYRDLHGHEVEGSLAANFLQRIILSEIEGRVKEASREKFTVRYVIQPVGSEKTTKYPLFVTSNKDKLSATERKQWYIRDINSSFSRSETDKKGWILKNGKDFSNGFQEKYISDNTDLFKIISSKYQLQFLDKALPNGAKKFGKDGDVNLKLSEHSFIAEETLQASWDFSTDTVSLKSVDKDLFVAIPVNIEPESQRSSREERTRYLGIDVGEYGLAYTLIETIPKVRVIKSGFLYEKTLRNIRKQVTNIKNSQRLGTFGVPSTKLARLRTQAITKLRNKVHDLVVRYDAIPVYEWQISNFETKSGRVTKIYNSVKRADTNSGANENADGDEAALVWGKRPYKIGQEVSARATSNMCSNCFKSIYQFTEQDFTEESRSGEYVEFKIAKNTVKGFVPNDEKITSKQKMINAVHDYARPSVDILVKRNDVGVDKEILVNFLEHRGASALYECPFCSHLSDADVQASLFIGLRAICRDQDDETTKVKISDLMDFAQKQNIDPISADFIQ

SEQ ID NO: 106 - protein, 1083 aa, synthetic construct
VELIVNSFFNNDGQLILDGESQFDFWEKEYNLDKGIIESAKPKGKYTDITFVIIPELISNLNTKQTLEDLINKRATWLSNRELDEDNKLLASILGFSDNANGFSNYLGVVLRGLQEEDGDKEALYEAQKTIFPRLENYRDKVLESLDFLSQKAKLLGETSLPLVNCWHDYRSVFGGKLQSWFTNFQKRKEELDGQISQFKESLFKAKSYLQKENFGEEADKEKEDILSFLSLLGNFFTDEQKSIKIEENYQLFEPLIALIRRRLNFFYQKYIQKEGDETKVNKFKEFEGLYKTIYKPVAFYGYSAKRANEKFVNKTVPILEDGIENIKKIIFYLQDSFSVQKTFEDVKQEREEIDDPYRKFLQFFWNKYLDASINSYLFLGKYEEIIKENVEDNKWKEISDKNKKGRYVFYKSPYTKGTLEKIPIKANSYLDKLQVSVLELVEFILSFKKDTLLSDSALLLDWVELSKNIISLLLKFNTKETYKLDGLLLENFSQAKKYQELFKREDYPKNEFSFIIQSLVLSEIRGAATLYSKREYIASYSVQVVNSDEKFKLYYIPKEEIPITPDTIKSKPDSIERKQLMKPHCFVVALDKVSEEKKNQNFNAILLSKSNTTAVFLPDESLQKLCKLSSSPYQLQFLDKYLFRPAGWENIDISLGEWNFVVEKGYKINWDLQTKKPKLLPITDSEQAKKNKIYLAIPFNLSPNNQIKQSAPLKSIAKGNEKKEKDLSRLNYPILGVDVGEYGLAYCLVKFDFDKNTYKITGIKLGSDEKETFGFIEDKNIGNIKDKFSEIQQKARQGAFDEEDTIVARVRENAIGHLRNRIHIVVTQEKSSSVYEDSITNFETGSGRTTKIYNSVKRSDTESGTKADEQIHNHIWGEKTKLVGRNVSAYASSYICVNCLKSLYQIKKEDLTKMTITQKDGRSRVVTISSPYGDIKGYVSKEEKYDVGYQFRGNDEDLKKLRKIVQDFARPPVSNRSEVLEKYAKPILETGRIEEWRKRRGNSSIFVCPFCQYVADADIQAAFMMAVRGYLRFSGIVPSLSNKNKQDQEKEKTAGESFLEQTQKQLKNIDINKIKSAFSIRI

SEQ ID NO: 107 - protein, 1113 aa, synthetic construct
MLEVLRKDVEHDSRHLYGASNLSDYQNKTVTYSVIDFWLDAIKAGGLFQPSAIKLKNFVSEENIKADSKLIAFMDQENLSTNLKSLPRAQTLENKDRLAKKVAMWFIEEFREKDFVLSAINEIVAGLTSSTGNQTKQKEFLLSYLGLNKDFFDKETTLTYFLHPDIPNLIDKGITVNELLEQRYKFLQTKTGENEVDIKKLQELLGLSDNFNALSHFLGKLWMTLKNEGVEEIYNQMLGISNLWQDQEDQLKTRLNALSTKAQLLPYPKMANHWGDYRSSLGGKLQSWSTNYFDYTQKSLQKLTAYKKDINLIISSLEKQKDNTEYMMLLKTAKESALMLNNLSDVPDKDRLNVLSATLAELRIGVNEVFQKSPSVFGYTKDEELLTFDQAFPVLTKPTPKYPEFFGDSKVNKFKSFERAKQTIENGVAVYNNLDVIMAKPNNLDETRILKFLDGWITYHSGCNIETIKSLVAELITSYSREPLENNRCRFYVNPNNPRNKLLKTVDVGKPDLSILLQKMVVLKENINRHIPESVTNNNCDYLIDWVEFCKRYYSFLLVGSTQTIDALSLCTDKFPQAEAYLKLNGTNLLPAPAVAKLINRFVSPEIKGAINLKSRKNITERNTLQMVGALDKWRLIVKLVDIKSKIANLPPQISTELQNLKTTDDVIQAKQRSIRLAKFLPHHFMLYVATRTPVPQETHALVYHKSSGSYRPGAISGDSLFKIASTPEQLQFLEMTLGLPKRYTNPQTQQDLLNIRIEEPSLITEHEYTDQTQAIHKPRVFVSVPFKIWPVYKNNVGHKTNKLGVDLGEYGVAYSVITPKEQGGKMDFVCLDKGFLYEPQFRAISAEIAETKARQLRGTFSMASSALADLRESAHKSIKNRLHFLATKHNAQLVYERQVDAFETAGNRVKKLYRTIKRGDVYPKQLTDEPIFKQYWGSYDKTNPPATEIGAYGTSYFCPKCRKSIYQLDKTIVVVELEKQNGYPGVYLVHTEPKDISFYIYCEKDLPNETRGLIRCGKLFARPPLESVAVDLCRHKGSLDRQFSSKALANRGSQPLFVCPFCLSCYDADIQASINIAIKRVLFENSGKNKDFNFYKESTADKVKNYPEAKLD

SEQ ID NO: 108 - protein, 1070 aa, synthetic construct
MAQQIRRSTYATGSLAHAQRVAYSGGNESVKTIKFPTEMQLKADSPEILNGIAEAIMQDDIAILGARTEYSHYYLAQQGKEVYSALDFWIDAVRLGMVFAQSAASLLQTVGVLSNAQTPIALMLGQSSVKELLNMHGAESFLAKRTFGKGSSVSGVSKNIIKCFVQGHITEEAAEAKAKKLLALGPAEQKQYKKQLLGIAGIAQPEGYRNTLGITACLLPGFETPDAYRAGRDVTLLYHVQRMQRLLSLDEVKEAYEFVGMHDSALSNFLNGNSNKGFLALLLRGEFDTLARGMMDMTPLWNEHNHDVLMNRLQALGRNAQKLSFTKPRFGNSFADHRKQISGKATAWFSGYCNKLDIAKEQIPLVLEDAKMFMEMLCAVEIDYEKEEFMHLQLSAFIERMERAHERLDADGVVALKKAQYVLPIIREFANPIVQREEAQQWLSLNIELVPDKRFTFKQAFPYLSDQGGADTEKGEQTKFQSVPRFFAEGAHAEYVAFSQAPMIFSVYMENLRLLWDRLIGMERRTPRNWEEHLSKLLYYLYVDIYVECRTDACKKYVRGILETYAHVSRIPEHVPGIKRFTIKPGYTPQGGLTKDQLVQCVLQIAQRLSNAGFEWKRISALERLDLVEVHKKAFAFLVGITHGSVDVSAYNWLNNKSVVQYLDVLKTTDLGGIRLARFLQSCVCATLRGSATRMSEQMLTARFSVQTATTIPQCELVYAVSEYMRKRRFVTPPSHVSRDVLDRKPASIVAQAIYNPEQMAGSMRFIPHRFGYQIQLPELARHESLNNALVVQKPTSTRRYSFARFDAEKGPVLWVESSHYQQQYFDWFFFAPNNRPADVIPQGVSLVVEQDIALQWDFDALVVHMQPVHEPRMYCPQPFKFVPRVESSVMQNRYMGIAPGTNEVAYAVIEVNGTRVSVINCGTFPLAGFANLSRAEEKKQKRERRVGSHAFSPDKRAIVLDNSANKLANQIHACAIQNRARLVWQWTPQTVQIAKGREVDMVYARARKSCAPKNDSDFDAMKTKWGKIWSAKWEDGKHTLSTEVYYAESLFQSACNAPVPPECATALMV

SEQ ID NO: 109 - protein, 1136 aa, synthetic construct
MAGFGPAICLIQFLCSPVYPKTNIIFSMPEEKDRPKKYKGLTGYKLHLKRYEYAGGGSTRTIKFPINCENNEYRQELIKKVKHDYKRLFGGMNITTWIQNPSCYSLLDFWLDTLRAGFIFGNTARELQKNIYYILYGKDVTEVEIKPKMVAKKPDFVSWLDWKSFCQEFVQKEGVRSRDGVTIETIKKKIKKCLKKEYQQTEDQQTSLELEKIAKGIFEKSIAEQEKFWRENYGLERLKKTGEITIFPLPDLDFDKNSKVETLIIKYIKLGKKMRVSVENLIGVGNNASGLSNYLNDIFNKLLNNKNEEIINNINSLNSAWKNKQTDLDLRLQFLSNKAKLLGKPKLVNNWSDYRTDIGGKIQSWLSNRDNQIKDIGEKLERQKAGLEKLKNEKVIGKIASNCLEYVTNSLGSNEIQTEDKLTIYREMVSDLRERVNFWQQENREVGEKYKTEIEAIKEKIKLVPLFVGKAKAEKYNNYLNCQNLINEGLAFLSEIEKITSKNNQVILTTEEKKKEINKLFSKYHQLNTSRFKGIIKKLFNRIGVDDISQYDPKKETFWENKHAKREKKIIEINSEKLNNFDIFQIAKDIAPRWEPKTMIDEEMIDAIEIEKIRWGWLTKTGKVKLIDKNLVYLTRARELIKSVAKVEPGLLIQRFVLAEIKGAISQKSRKFFTVRYVINPGKFNEKFPLVYVPKNGKKISRTNAKYWGYVKKEDISCSKNSEKIAYYWQDKSNRPKEGRLTTTDSLLKIQTSKYQLRFLEDVFCDNRSKTWIKHAPTISSPALILEETVKVKWANNQVKLEIAKQQLYMSQPIVFGYVDNKTRERLENQTRVLGIDVGEYGLAYSIIEKRESKIKLIKKGFIYEPALRLIRDNIAKNKLSQIKGTFSMPNTKLQRMREQAISGLRNKVHDLVVRYKARPVYEFQISAFETGSNKTAKIYNSVKRSDVSESKPNNIDKTIKNTIWGSTFVKVGSSVDARGTSRLCSSCKKSFYLYLHDDKKSKTKYKVENSQIKLDNQNWNIYNKTGANVLNSKEASTGLYDYMRPPMKTFLKRQERSTIKYTDKELEYFQKRRGDSAIFTCPKCGHVSDADIQASLIIGLKEFFNREYSSQKQNFEKFMKYVSGLEIPTVGLEVD

SEQ ID NO: 110 - protein, 1246 aa, synthetic construct
VKAITGYRLHKERLLLSGGGVVRTIKYPIVPLNHASQKSKEFLKQFERGIIEDDLKIRGSLNLNDYLRDKDCYTLFDFWEDSLRAGVLWMPKVNDLIDFFNQEHIDHIWQNASSKITYYFEKDKFKEILKRIPVRKTKKRANFLKDLLSSVKKDFLQKGKDGEYEVINQEVNTLIGSIVNTFYDKDGRLVLEGEKNQSKFWKEEYGLDVYYLKQIQPAGDYQKITFVIVPELIELEKNSSLENLLDKRREWLLSCFKKEEVKKEELEKILGLSDNFNAFSNYFGVVLQGLQNGKTTEIFEAHKTIYPEVEKDSDKIKKALDFLAEKAKKIGKTKLSGVNGWHEYRSIFGGKLQSWFSNYQRRKNELGEQIDKLKIGLDKTKNYLEDTKFDSDEESRQKKEHILSLLARLKEVTTEEGGKSIENEKDYLLFNDLLAQVRSNLKFFYEKYIKEEGEEKPVNQFGEFKYIYQTIHKPVAFYGDVAKTKNEKIINKTFPILKDGIENIKKIVIYLKESFFPEDFLDQKSSQETYKKSLQFFWNKYKENALNSLEFRNYYREILKEATADDQWQELQKKENKGRYVYYKSPYAKGTLREIKTKEVKDFSERVTELYFRLAEKVLSYEKNNLLKDEKILLDWLEIAKHIVAKLLDYNTKERFHLKNLSVDNLEKLKRYVDLFRGDGYSKSEYGFITQNLIFSEIRGAAALFSKKEYTAKYSVQVVSSDNKFKIFYFKKGENEIDKSYLEERKKLMLPHHYAVALREVKTKKKGGGNSIVLEKNGFYPVFVNHDSKDYLFRLSSSPYQLQFLDKFLYHPKGWEDIDIALSEWSFVLEKRFSIKWDLQEEKPKFNPINEGKKAQKNKLYIAIPFNLTPSKQAQKNAPLKIIAKKGNKRDLSRIDYPILGVDVGEYGLAYCLIKIIYEQKPQNVKAIEILKDEKEKPVCGFIEDRNIANIKDKFSEIQKRSKLGSFDEADTVVSRVRENAVGNLRNKVHAVITINKGLSLVYEDSISNFETGSGRTTKIYNSVKRADTKFETEADKAEHSHVWGKKTAIIGRDLSAYASSYTCIKCSHSLYEIKKSDLPSMKVIDVDGRILAITTPYGNKTTIRAYTSEKEKYKTGYQFCPNDEEFKIFRKMVRDFARPPLSRNSEVLLKFASGLTEKNKIEMFKKSRGNSSVFLCPFCGFIADADIQAAFFMALRGYLRFSNTVSPSGKENNEKTTWENFLEKTQKILQKVEGKEITKYISLRF

SEQ ID NO: 111 - protein, 1153 aa, synthetic construct
MGNSRKYRGKTGYTLHAKRIEYSGQSKALRTIKFPLITNSGVLFSALHDRVQHDHLYLTGSTNIDDWINHGEIYSLVDFWVDALRLGGFFQPDAKSMGRLLDLMEGRNTIYIQMVETMPTSLRRQIDTHQLINDLLKTTGFRKSSSPERIFARVVQAFKEEYRESQIARQTASIISKGVFEKDYGEEQVRFIAQAFKMKYIADDRSLTFFLNPYLEVDQESSPALLVRMLEDYFTPYIEDGITNLDDLLGYGNEANAYGNFLGQVVPLLQAGKVGEIVDAFGKHSALWLGRERELRQRLQYLAERAQRFHKPLLVNSWADYRSVFGGKLQSWYSNYKRHMLQVRERLELQKPEFERVEFEINKALRENDDRYLRETKAAVEELGKILEALLEKYQIGGETIYEDDLMMYRDLLGQLRVRINSLWQRRRIFKDKKDGSESKPIHKQLRAIFRNMRVVPSFFGRSKFERNRRFLDSVRRLEEGFAILAVLRDRMLAKPLDWEGVDDKYLCRQLDTMARMYRRGGTNLFTSKLTKILVDYGGKVPGERERFYVSPYSRERFHLVAPAGRKKLVPEALVDWWEELKFKGNWKSITDKQLVDLLEIEKVRMGWLLRLHPDDEWKLDDLRPKYFEKARSLIEINNGRLSSWQFGQFVQAFILGEMRGTASLMTRRRFAVHHVVQPLESEKKFVLQAVFKNGETNEFGPGEQYVFAVTVPRLETGVPAEESGLTSDYVVPIREKEVSNKEALCRNGGPKKLTDTFRIGGSRYQRQFLEWFVMPHRKKGRVSLKRSEPMVVAETDYLSVWDKTSGGVKLTPRARRLFYNQPFAFTPRGLDTMIDHLPGRYLGVDIGEYGIAFAVIQVQEEDGKVELIDRGFFFDRAVRKIRDRVNELRKRQMTGTFAIPDTAVARVRENAIHTLRNRIHDLVLRYKAKPIYEFEVSAFEVGSGRVRKIYHSVKRSDVPSDIKADGLERKAIWGNQYKDGLVGNHIGAYATSQTCSRCRRSVLDWVDREVNYRVEIDPRTRRGKVWLSDGVKIEIFVDDSVDPDEVTKISGKEVNKYAYKFMRPPKDSPFLENRGVAVSEAFKQHRGNQAIFVCPFFDCHHISDADIQAAVHIAFKGYLKDKEKRNKTRYDFFDELGNLSLYPEVGFDDSLF

SEQ ID NO: 112 - protein, 1146 aa, synthetic construct
MEKSKRITGYHLHIARSLYSTKTIIRSIKFPLKSPSHDAGILFAENIRNDYLKQVGNIHPSAYFASTIEYSLLDLYVEFLRQGFLFAPTATKLKSKVYKITYGKSLAEVDIFPNISQEFLNLVSKDLFDKTILQNSTVRNTSTKASLDKRYTSIFKKEVLRETVLEQVTKMVDGVYRKNYEEQKQFWKNFINLEFSEKESNLTCYPLPNKGDKFSGKTPIQEIILSHFESKKKDLFGTQRQDLTDEELKLARIELLEDIGLSANFNALSNFLGSIVPLLLTEKGIRELISKILNYSTVWKDKEEILEGKIAILALTARKISLPSLTASWADYRSSFGGKIQSWVSNFLTQNVKLREALNEQNKEIQELKKEYTIDSSRVIEINRQLLGNLTNQSQYQLYVDSLAYLNTEINMLRQERDVDFKLFQDKYKALSSNLPKIPNFIVQRKFTLFQDFQNSLVKLEQGFSYIHSVYNELLGDIRVSQNPDIDGVLVRKTLDSLKRFYCYLEEGSYYKNVFKEFFLSIGHTVSEIEGTDKRFFIAPQSRDKQLSLIIVSESTFEIIKDKLLKIPIDFSWKHQSISELLAITEVMKIITAFYLKMIPNTKEFTLDENTILHFPAGQRYLKTLGKSLNIKHFSFFLQRYIFAELRGCLNLLSRETYIERYVVQTLGSDKSFPLVLFPKDKEQKPSITNRAYWGIVSQRPSRNQKDQDDTLNGYIFKSKEYNQGAGSFKENTKIFPNEAVFQIRSSKYQLQFLDNPLNDLGDRSLWKRVSVKLSEHSFIVEDTMRVTWDREKMRPNISCIKTDIYVSIPINISPNIKPHAVNPSRYMGIDVGEYGLAYTIFETNPKFKILKQGFLYNANLRKIKSHIKRGKEKQVRGTFSSPSTSIQLIREQAITSIRNTVHDLVIRYKAKPVYEWQISNFETGSGKISKIYNSIKTSDTNSDTDAEKGAKELVWGKGTKTIGKEINAYATSTICSKCFKSYLLEIGLKDSDKFTLEKIGNETFIKINNTLIRYFSRKPITNTDDAKSAIYGTMRPPQYSEIVKSLDIEDRFFSTRGNSAIFYCPFCKHISDADIQASLFIGLKGYLKDLEKKTETTKNNTQVDRKTKSLDETRRMIKEARLLVKDPILFDRNNFLSFGEHLVNT

SEQ ID NO: 113 - protein, 1026 aa, synthetic construct
LILEGKKQDNFWLNEFGIDKSIIQKTKPEGELKDITFVIIPELIVDSFNKEYEVDSLIEKRRVWLKKRFNKEKEIEKNLQLILGLSNNFNGFSNFLGKGLRAFQGGKILMIFEAMSKINPSIKNQENKEKVLEALNFLSDKSKFFPLRPSLNIVKSWADYRTFFGGKLQSWYSNGIRRKNELKVQVKEIYEFLTKAQDYLAAKISFNDENKRYARKELESINLKINRLKQFIENENFDISSEDRYLIFDTLLSSLRTQLNLYYQKYLSSEEDNIRENKDLKGIYQKIYKPIAFFGKATKRKNKKVIEETVPIIESGINNLFSLMKKLEKTFLPKNTFSKVKNKNEDEETNLRNLVDYYQNKVSHKMLNSLTFVNKLEEVMKSVIEENDWDKLHSNKYVFYKSEYQKGALELIPLKKGSWIDIFEKIILEMTSYLLTFDLTDLLKDKKILLDWIEIAKNTLAKLIKFNTCDVFTLDELNLDLRNFPKAMDYIKIFKITKVDKNELNFIVQSFILSELKGAATLFSKEKYLAKYNVQVINADKKFKLFYKPNDGFIEREVDRKNLLKPHQYFVALDKIEDKKIKEKANFLLITKEDIKPVFIKEENFSKLYKISSSFYQIQFLDKFIYMPEEFKDLGIRLSEWNFVLEREYKIDWDLCTKRPKMTFIENSKKNKLYLSIPFNVFYKSKKKDVSLSKVISNRLSYPILGIDVGEYGLAYLLAEFSDKKIKILKKGFIEDRNIANIKDKFAEIQLKARTGIFNEEDTTIARVRENAIGNLRNKVHYILTSDRGASIIYERSISNFETGSGRTTKIYDSVKRADTEFETEADKFIHNHVWGKNTKYVGRSLSAYGSSYICSKCHRSIYQFKKEDLREIKLLMREGNILTFLTPIGKVWGYSKDEKFKKDYQFKPTEKDFKEFIKILKDFARPPVGKNKTEVLEKFFLKDNDKKAKIDEFRKKRGASAIFICPFCGFIADADIQAAFVMAVRGYIRFKESQIKEENKSLILEKTINYLKEVQFKPEDIFLPF

SEQ ID NO: 114 - protein, 1043 aa, synthetic construct
MSKNLTGYRLHLKRILFSGGEITRTIKFPLLTHNDDVFIKNFKKNLIDDDLKIRGDLNLNDYLKYTALGNPIYTIFDFWTDSLRAGVIWADKPSSLIDFIGDYYQNEKSPCNLVWEKANEEIKEYFDKVKFKEILISDPLRSTSQKDSFKNRLIKCLKNEHFEKKEKNFLVKSLAAKDIIERIANAFFDDKGNLILEGKEQNKFWLDNFGIDKSIIEKAKPKGELADITFTVIPELIEDLEKECDVEYLVEKRRFWLKKRFNDTKEVEEKLEKILCLSNNFNGFSNFFGKALRLFQKGEIEIIFRAITKIYPQLESPENKKRVFKCLSFLSEKSKLIYKEPSLNMVKNWADYRSFFGGKLQSWYGNTLRRKKELDDQIKNFKNSLNIAKKYLFDTNLNQEVDEDQLKIIRLKIERIEEFLNKYNIVSEKNYSLFYEILSSLKSHLNLYYQKYILKGDEEKSIEKDDHFSGIYEKIYKPIAFFGKASREKNKKIVESTIPIIEDGINNILLLINYLKPSFSPRDTFEKVKNKDETEEIILRKLLDYYWNKRKNKIINSIDFLNKIEEIIRENIDEKKWQNLQIKENWNKYVFYKSRYQKGLVNQIEIKTSNFLEVFEKTVLGLTSYLLNFNLEQLLGDKKLLLDWVETAKNILSKLIRFNNKESFVFDTLGINLKNFPKARDYIEAFNIKEVSKNELGFIIQSLIFSEIRGAATLFSKEKYLAKYNVQVIGPEEKFKLFYQPKDNLIEDDRKKLMKPHQYLIALDEIKEKNLKDKFNFLKLLKNEIKPVFIDEKDFSKLYKISSSVYQIQFLDKFIYLPEKWKDIKVSLSEWNFIVEEEYQPKWNLRDKRPEFKLIEDSKRNKLYVAIPFKISFKGKKDNVVIKRVIDKRLEYPILGIDVGEYGLAYALVKFFEDKIEILDKGFIEDKNIAKIKDRFREIQQRAKSGIFNEVDTVVSRVRENAVGNLRNKVHFVLTDKEGASIVYEYSISNFESGSGRTTKIYDSVKRADTEFESEADKQIHAHIWGKNSKYIGRNLSAYGSSY

SEQ ID NO: 115 - protein, 1095 aa, synthetic construct
MSKKVSLSGYRLHVKRLHFAADTPTMRTFKFPLVRKCGKKADFTDVPQELIEAIKHDDLYIRGPSNLVDYLRFRNKKIYTLIDFWIDSLRAGVIFMPSTRALADFVTRIKEGKTIFEKIVEEADLRFKQYFNVSKFINEFVAKKEMRKGKSRDSFVNTLAKILREDLSEREKIGTERFIAELADVFFDARELKILGFNAQNEVWQARFGLNKEAVDLDFDLTFFIFPELASKQKVFITPENALEIFEQLIQKRISWFVAHGYSKEKAEREVDRVIGLSDNFNAFSNYFGEVLSILKNNRVEDVFKLLTKLYPKIGEIEKRQLAWLAERLQGLEYPLMPGTNGWHEYRTLIGGKLQSWWSNHKNRRQEQMEQIERLKRELPNLQEFVKHYPENELEIDEEVMISRKIEIEELVEKISEVIKQPDNIFTDLYRYEFIETLIRQLRQRLNEYFQLYAKGDDDKSPEDNKYLKEFWKIKIYKPRSFYGYAQRERLKKIVETTIPTIKQGIKLTQHLLLTLPSFVEYERASKEKEGGKKEARSDIGLRRFLEALRRKIKDDKLNTKIFKDLYINILQPYLPNEDISLLTDKRKLNRYTFYKSSYSTSTQKELELPAIDYSKEAENIIKEIVEFLVELKEEKFLKEPGLLLDFIETSKVVIARMFRWMDQEEEWHLHQFKFDIFPAAQKFVKELKEEKVKTPMLRFIIMSQVFSEFRGAATVFSKKYVTAKWNVQVVGSDKKFKLAITPKDKHIDVNNLHKVLAVNDPEVLKNIKWNVVISSKVGNLQRMITLKNKNGEFGITKEKIDEGNLLQICTSKHHLQFLERLIYKPQNWKNIKIKVSEPSLIVEERYQLTWDLKTALPITTSHNVNKSRLYYALPLQFEVERSKRSQLAEIKFHFEVNPSLALKKYYFLGADIGEYGIAWVVLQVDEGTREIIVKDWGFEYDKQIKKIQDEFREIQQKARLGVFLEDTTLLAKLRENAIGYLRNKLHDRLIDYGGHIVYEANISGFEVGVGRITKIYDSVKRADVGNHPDMGRDVGKKLVQHIWGLKIGGKKNSPGWQVGAAGTSYTCICCGQSIYQLGNNKKNKLKEDKDKKDK

SEQ ID NO: 116 - protein, 938 aa, synthetic construct
SQLIDERKKYFSEKDLEEILGLSDNFNAFSHYFNKFFLLLYQDKQEKIFECYQKIFSFSGEDRKRIKDALDFLSEKSKLLGLPKIVNSWSDYRSVFGGKIKSWFSNYLNREDKAKKQEKKIKEGLEKVNKFLLDFIQKNQVDSDLQQEIKFYYDKLNQFINSYQNQEFFHQQELFLLFSDLLAEYREKLNRFYQKYLSDKEKEEKKVDEFPLFKDLFEKYEGPISFYGKTKLEDNKKIIDLTFKTIKVGLNLIRRLLLDLYNSFDFKNSDNNNQERDLRRIFELLLNKIPATKTFREKYLSILKDNFDQKTYKDITLKPSQYTFEENIYSRENRKLIELPSKNFEELLSKIIKDLTDFSLSFKNDDLFADIYLLSDLVELAKTLISLVINYSNKSQFDSYKNELIDDTYQKAKKYLETFKISFFNSKKEANYFYQTRVLSELKGAVPLFSKKYYQAKYNIQILKSNEIFPLFVKFSDLLKKEEINDINKLKLIFKKPYRYLIALKKIKFKKNQQQSSVIHLDKKNKDLVLISPQDEDFLFKLTSSFYQLQFLDRFVYPVKKWLDVDITLSEWSFILEKKYKINWDFNNGKPEFSEIDSRLYLNIPFKIKAINQQKILKPKELFLGIDVGEYGVGYALVNFKDEEIKIIKSGFIRSKNIASIRDKYRLLQDRSKKGVYFSSTNVVQEVRENAIGEIRNQIHDILIKNNADLIYEYNISNFETGSGRITKIYDSIKKSDVYAENEADKSVIQHVWGIKKSIASHLSAYGSSYTCSNCGRSIFSFSENDICSSKVIKRDGNIITIQTPKGEVFAYSKDKKFNVGYSFSQEKSKEEMKNLFMKIVKAYARPPLLKSEVFLAQKKLDKEFLEKFKKERGNSAIFVCPFVDCQSLADADIQAAFIMALRGYLKKKKGKDINYLEESLNYLQNFKGKINFSNLLH

SEQ ID NO: 117 - protein, 934 aa, synthetic construct
EPDLDPIACFDRLKNIMSVEEWKDVYSLLGIDDNCNALSNYFREVLEDFQQENTQSLKKKFKNVIHVWNEDQSGLEERLHWLEKQAQKLSKSSLAKSWADYRSNFGGKLKSWLSNYHRQKQLIEDGLKNQKEEFQKIDTQKDSPDFQQEGSVKIAQLSKQLLKIINETLNKEINLTYQRFFLYRDLLAELRTQLNYYHQAQDKSEELVREVYPHIFKDLELVPQFFGKAKTQRYQKYIKTAELIEQGFNFLNNNFTSFSTNQKQIETDVLRKKLESLHRYYFTANTNRYKPLIETILTKYTETIPPENHKYFKHPRSTDPRTKQIELNLENTNTINDLERIQKNLNLESDWKHLSAAQLIDYIELLKIKWSIAVLLSDQKILVKPDELSLFDKASAYLSIFGNSIPSEKISQFIQSFVLSELKGAVNLLSRKEFITRSTSQFIESHEKFPIVYLPLDKKFGKDHIPEELRTEFPKTAEELLGDKSKIPDFKILSYYPHKWAVIDRDHIKNTPVENYIPALVIQGKKRDEFKVKYVDKNELLQIRTSTYQLQFLEKFLRPPAKWRDIQLRISEPSLILEEKFKVSWDRQNLQVKLDPAGSRQLFYSLPFNLEPQIDTAARSKAIDNRNAYLGIDVGEYGLAWSCLKFNQESKCEQLGNGFIFSHELREIREHVANMKNRQLKGTFGMPSTSLARIRESVIHSLRNKLHDLALRYNAKLIYEMQISNFETGSARVSKVYRSVKTADVHPENDADRAVMEQTWGKGTKYMGTHLSAYATSQTCSKCHKTIADDLDWESSFEVKRQVEGSIFELQNNQEIFKVYSDRKLEIGQQLDEKTAKNLALKFMRPPAESDVLNWKNVSVPEEFTATRGNSAIFVCPFCDHVADADIQAATNIAFRGYLRDKYKEEKSDERFDYIGKIKKHAEKNEYPLNELRV

SEQ ID NO: 118 - protein, 1139 aa, synthetic construct
MPTGYSLQTKRLLYSGGKATRSFKFPLVPFSQKSEAAYKDFIGEVRQDAVNMMGALQLFDWKQKGETYSLADFYLDSLRAGVIFAHKFDAMKSVVLALGGGKDPQAFFAEQLRAHHPDLADLVDMGAFYNFCTSNSRQRNKTKEELLKILAKSLFTKTSWKDQSDRVAIYVGQITEWVYDKTNAQKSDAAKEFLGFNPKPESNFDFTAVISLDLPFVPDMSAYNLVERIGAYIEGQAAYTQYKAEDRLSAVLGLYNKQGAFSNFFNEILSDLKKEKGHTWFTAMTQALPAWQKREPELRERITFLAERAKQLTAPALARGWHDYRTSLGGKIESWASNFVRQKDEIRKQLFGHETITERGHDKRKKIWRNGHKQTLENILEDDIVKKHSDAVALACAELKCIATMELHITDALLSLYSDQLGQLRIALNEAYQAEYPENLEKNGKERKQWESGHRANRKYAQIYDDIKLIPRFPGEAKYEKYRKLVLAPKLVKTAVAYLQQSSEMLRKQSCPEHADEECLQRMLETLRRKYFVLNGTIAAHMIRNALKPFACSVDGKPVTMDDVLNEKQRENNKDYWYFWRAKQARARFGRKIDMKYDKNVDAALTKIAVAIAPRWTDRIASGDIGDLLDACELEKIRMGILVDATVGDTWPLGLDTLPGEMFEKAHAFKEVIGDRPATRAEFGRFLQSCVLSELKGALNKMSRKEYTERYVVQPIASHEKYPLFNHEKQWGIALDKKTEGAIKISQKSKAKGFDDKAFIMEPQREERLLTIRTSKHHLQFLQKTLGGKDSWWHRKKMGIENGEYSFIAEVPMRLRWDFGAETLQIEEADKPRLFVSIPFTLTPEQKKEESHLAAGKRKRYMGIDVGEFGLAWTVLEMRGDAPHTVGHGFIYERLTRNIRNYVQTLKTRQMRGTFGMPSTKLERLRENAITSLRNQVHAIAMKYDTIPVYEAEISAFETGGNKVKAIYDSVKRADTGRGGIEAEKMEADLVWGKWNGKGNAGKQIGAYATSYLCTKCLHSLYDKTPEADRSAEKKTTRPSLDDERVRKMGLPETWKNQRGNSAVYVCKCGHVSDADMQASYWIALKRALRDKKYGEQTDDSNEKKTVEIDDLRAAHRENPTVITLQSDGSVHYAPSSAL

SEQ ID NO: 119 - protein, 1256 aa, synthetic construct
MTKRKSRLAGYRLHRERILFSGGQVIRTIKFPLAPLNPSEETALFLKDFERTTIADDLKIRGDLNINDYLAYTAEGKPLYTLFDFWVDSLRAGVIWMPKGTKLIDFLASQYNTTSPFDRIWEKASPRITSIFKKDKFKGIFLSDPIRNTSTKNSFYKRILESLKDDLVKENIIISTDAKKEVELIVNSFFNDDGKLILDGESQFNFWEKEYNLDKDIIESAKPKGRYADITFVIIPELICDLNTKQSLDNLINKREAWLSDKKLLSSILGLSDNFNGFSNYLGVVLRGLQEEDGDKEALYEAQKTIFPRLENYRNKVLESLDFLSQKAKLLGETSLPLVNGWHDYRSAFGGKLQSWFTNFQKRKEELDGQISQFKENLFKAKNYLQKENFGEESDKEKEDILSFLSLLENFFTDEQKSIKIEENYQLFEPLLALIKRRLNFFYQKYIQKEGDETKVNKFKEFEGLYKTIYKPVAFYGYSAKRANEKFVNKTTPILEDGIENIKRLISYLQDSFSVQKTFEDIKQEKEEIDDPYRKFLQFFWNKYVDASINSPLFLEKYEEIIKENIEDNKWKEISDKNKKGRYVFYKSPYAKGTLEKIPIKANSYLDKLQVSVLELAEFILSFKKDTLLFDSALLLDWIELSKNIISLLLKFNTKEAYKFDGLLLENFSQAKKYQELFKREDYPKNEFSFIIQSLVLSETRGAATLYSKKEYIASYSVQVVGADGRYKLYYIPKEEIPITTDTIKSKPESIGRRQLMKPHCFAVALNKVLEKKKNQNFNAILLSKSNITAVFLPDESLQKLFKLSSSPYQLQFLDKYLYRPAGWENIDISLGEWSFVVEKKYQINWDLQSKKPKLLPITDSEQAKENKIYLAIPFNLSPNNQIKQSAPLKSIAKGNEKIEKDLSRLNYPILGVDVGEYGLAYCLIKFDFDKNTYKITGIKLGSDEKETFGFIEDKNIGNIKDKFSEIQQKARQGAFDEEDTIVARVRENAIGHLRNRIHIVVTQEKSSSVYEDSITNFETGSGRTTKIYNSVKRADTESGTDADKQIHNHIWGDKTKWVGRNVSAYASSYTCVNCLKSLYQIKKEDLAKMIIIQKDGRVVIISSPYGDMKGYVSKEEKYDVGYQFRGTDEDLKKLRKIVQDFARPPVSNRSEVLEKYAKPILETGRVEEWRKRRGNSSIFVCPFCQYVADADIQAAFMMAVRGYLRFSGIVPSQSNKKKQDQEEEKTAGESFLEQTQKQLKNIDRSRIKNAFLLKI

SEQ ID NO: 120 - protein, 1184 aa, synthetic construct
MFNQKKGYRLHLERIIYSGGEITRSIKYLLASYSDSQKHKEILNNFSQGLYNDDLKIRGCLNLNDLVNNNQIYNLADFWIDSLRAGVIWQSSASSLIDLIKRLNHQETIGEKIFNNANERIKRFFNSEKFIKEIILSDPKRIGSKKQAFYNSLFDILKDEFKKQEKNEKIILDNQAEQLIKEIIDAFYSNDGTFIMEGEEKQNNFWQEKFNIDKNIIKKEKEDILKDVGDITAFIHPSLLSLKSDVSQLIDERKKYFSEKDLEEILGLSDNFNAFSHYFNKFFLLLYQDKQEKIFECYQKIFSFSQEDRKRIKDALDFLSEKSKLLGLPKIVNSWSDYRSVFGGKIKSWFSNYLNREDKAKKQEKKIKEGLDKVNKFLLNFIQKNQIDPDLQQEIKFYHDKLNQFINGYQNQEFFHQQEIFLLFSDLLAEYKTRLNFFYQKYLSDKEKEEKKVDEFPLFKDLFEKYEGPISFYGKTKLEDNKKIIDLTFKTIKVGLNLIRRLLLDLYNSFDFKNSDNNNQERDLRRIFEFLLNKIPATKTFREKYLSILKDNFDQQTYKEMTLKPSRYTFVENIYSRENRKLIELPSKNFEESLSKIIKDLTDFSLSFKNGDLFVDIYLLSDLVELAKTLISLLINYSNKSQFDSYKNELIDDTYQKAKKYLETFKISFFNSKKEANYFYQTRVLSELKGAVALFSKKYYQAKYNIQILKSNEIFPLFVKFSDLLKKEEINDINKLKLIFKKPYRYLIALKKIKFKKNQQQSSVLHLDKKNKELVLISSQDEDFLCKLTSSFYQLQFLDRFVYPVKKWLDVDIALSEWSFILEKKYKINWDFNSGKPEFSEIDSRLYLNIPFKIKAINQQKILKPKELFLGIDVGEYGVGYALVNFKDEEIKIIKSGFIGSKNIASIRDKYRLLQDRSKKGVYFSSTNVVQEVRENAIGEIRNQIHDILIKNNADLIYEYNISNFETGSGRITKIYDSIKKSDVYAENEADKSVIQHVWGIKKPIASHLSAYGSSYTCSNCGRSIFSFSEDDICSSKVIKRDGNIITIQTPKGEVFAYSKDKKFNVGYSFSQEKSKEEMKNLFMKIVKAYARPPLLKSEVFLAQKKLDKEFLEKFKKERGNSAIFVCPFVECQFLADADIQAAFIMALRGYLKKKKGKDINYLEESLNYLQNFKGKINFSNLLH

SEQ ID NO: 121 - protein, 1184 aa, synthetic construct
MFNQKKGYRLHLERIIYSGGEITRSIKYLLASYSDSQKNKELLNNFSQDLYNDDLKIRGCLNLNDLVNNNQIYNLADFWIDSLRAGVIWQSSASSLIDLIKRSNHQETIGEKIFNNANERIKRFFNSEKFIKEIILSDPKRIGSKKQAFYNSLFDILKDEFKKQEKNEKIILDNQAEQLIKEIIDAFYSNDGTFIMEGEEKQNNFWQEKFNIDKNMIKKEKEDILKDVGDITAFIHPSLLSLKSDVSQLIDERKKYFSEKDLEEILGLSDNFNAFSHYFNKFFLLLYQDKQEKIFECYQKIFYFSQEDRKRIKGALDFLSEKSKLLGLPKIVNSWSDYRSVFGGKIKSWFSNYLNREDKAKKQEKKIKEGLDKVNKFLLNFIQKNQIDPDLQQEIKFYHDKLNQFINSYQNQEFFHQQEIFLLFSDLLAEYKTRLNFFYQKYLSDKEKEEKKVDEFPLFKDLFEKYEGPISFYGKTKLEDNKKIIDLTFKTIKVGLNLIRRLLLDLYNSFDFKNSDNNNQERDLRRIFEFLLNKIPATKTFREKYLSILKDNFDQQTYKEMTLKPSRYTFVENIYSRENRKLIELPSKNFEESLSKIIKDLTDFSLSFKNGDLFVDIYLLSDLVELAKTLISLLINYSNKSQFDSYKNELIDDTYQKAKKYLETFKISFFNSKKEANYFYQTRVLSELKGAVSLFSKKYYQAKYNIQILKSNEIFPLFVKFSDLLKKEEINDNNKLKLIFKKPYRYLIALKKIKFKKNQQQSSVLHLDKKNKELVLISSQDEDFLFKLTSSFYQLQFLDRFVYPVKKWLDVDIALSEWSFILEKKYKINWDFNSGKPEFSEIDSRLYLNIPFKIKAINQQKILKPKELFLGIDVGEYGVGYALVNFKDEEIKIIKSGFIGSKNIASIRDKYRLLQDRSKKGVYFSSTNVVQEVRENAIGEIRNQIHDILIKNNADLIYEYNISNFETGSGRITKIYDSIKRSDVYAENEADKSVIQHVWGIKKPIASHLSAYGSSYTCSNCGRSIFSFSENDICSSKVIKRDGNIITIQTPKGEVFAYSKDKKFNVGYSFSQEKSKEEMKNLFMKIVKAYARPPLLKSEVFLAQKKLDKEFLEKFKKERGNSAIFVCPFVECQFLADADIQAAFIMALRGYLKKKKGKDINYLEESLNYLQNFKGKINFSNLLN

SEQ ID NO: 122 - protein, 1184 aa, synthetic construct
MFNQKKGYRLHLERIIYSGGEITRSIKYLLASHSDSQKNKELLNNFSQDLYNDDLKIRGCLNLNDLVNNNQIYNLADFWIDSLRAGVIWQSSASSLIDFIKRLNHQETIGEKIFNNANERIKRFFNSEKFIKEIILSEPKRISSKKQAFYNSLFDILKDEFKKQEKNEKIIIDNKAEQLIKEIVDAFYSNDGVFLMEGEEKQNKFWQEKFNIDKNMIKKEKEDILKDLGDITAFIHPPLIILKGDVSQLIDERKKYFSEKDLEEILGLSDNFNAFSHYFNKFFLLLCQDKQEKIFECYQKIFSFSQEDRKRIKDALDFLSEKSKLLGLPKIVNSWSDYRSVFGGKIKSWFSNYLNREDKAKKQEKKIKEGLEKVNKFLLDFIQKNQVDSDLQQEIKFYYDKLNQFINSYQNQEFFHQQELFLLFSDLLAEYREKLNRFYQKYLSDKEKEEKKVDEFPLFKDLFEKYEGPISFYGKTKLEDNKKIIDLTFKTIKVGLNLIRRLLLDLYNSSDFKNSDNNNQERDLRRIFEFLLNKIPATKTFREKYLSILKDNFDQQTYKEMTLKPSRYTFVENIYSRENRKLIELPSKNFEELLSKIIKDLTDFSLSFKNDDLFVDIYLLSDLVELAKTLISLVINYSNKSQFDSYKNELIDNTYQKAEKYLETFKISFFNSKKEANYFYQTRVLSELKGAVALFSKKYYQAKYNIQILKSNEIFPLFVKFSDLLKKEEINDINKLKLIFKKPYRYLIALKKIKFKKNQQQSSVIHLDKKNKDLVLISPQDEDFLFKLTSSFYQLQFLDRFVYPVKKWLDVDITLSEWSFILEKKYKINWDFNSGKPEFSEIDSRLYLNIPFKIKAINQQKILKPKELFLGIDVGEYGVGYALVNFKDEEIKIIKSGFIRSKNIASIRDKYRLLQDRSKKGVYFSSTNVVQEVRENAIGEIRNQIHDILIKNNADLIYEYNISNFETGSGRITKIYDSIKKSDVYAENEADKSVIQHVWGIKKSIASHLSAYGSSYTCSNCGRSIFSFSENDICSSKVIKRDGNIITIQTPKGEVFAYSKDKKFNIGYSFSQEKSKEEMKNLFMKIVKAYARPPLLKSEVLLTQKKLDREFLEKFKKERGNSAIFVCPFVDCQSLADSDIQAAFIMALRGYLKKKKGKDINYLEESLNYLQNFKGKINFSNLLH

SEQ ID NO: 123 - protein, 1184 aa, synthetic construct
MFNQKKGYRLHLERIIYSGGEITRSIKYLLASYSDSQKNKELLNNFSQDLYNDDLKIRGCLNLNDLVNNNQIYNLADFWIDSLRAGVIWQSSASSIIDLIKRLNHQETIGEKIFNNANERIKRFFNSEKFIKEIILSEPKRISSKKQAFYNSLFDILKDEFKKQEKNEKIIIDNKAEQLIKEIVDAFYSNDGVFLMEGEEKQNKFWQEKFNIDKNMIKKEKEDILKDVGDITAFIHPPLIILKGDVSQLIDERKKYFSEKDLEEILGLSDNFNAFSHYFNKFFLLLYQYKQEKIFECYQKIFSFSQEDRKRIKDALDFLSEKSKLLGLPKIVNSWSDYRSVFGGKIKSWFSNYLNREDKAKKQEKKIKEGLEKVNKFLLDFIQKNQVDSDLQQEIKFYYDKLNQFINSYQNQEFFHQQELFLLFSDLLAEYREKLNRFYQKYLSDKEKEEKKVDEFPLFKDLFEKYEGPISFYGKTKLEDNKKIIDLTFKTIKVGLNLICRLLLDLYNSSDFKNSDNNNQERDLRRIFEFLLNKIPATKTFREKYLSILKDNFDQQTYKEMTLKPSRYTFVENIYSRENRKLIELPSKNFEELLSKIIKDLTDFSLSFKNDDLFADIYLLSDLVELAKILISLLINYSNKSQFDSYKNELIDDTYQKAKKYLEIFKISFFNSKKEANYFYQTRVLSELKGAVALFSKKYYQAKYNIQILKSNEIFPLFVKFSDLLKKEEINDINKLKLIFKKPYRYLIALKKIKFKKNQQQSSVIHLDKKNKELVLISPQDEDFLFKLTSSFYQLQFLDRFVYPVKKWLDVDITLSEWSFILEKKYKINWDFNSGKPEFSEIDSRLYINIPFKIKAINQQKILKPKELFLGIDVGEYGVGYALVNFKDEEIKIIKSGFIRSKNIASIRDKYRLLQDRSKKGVYFFSTNVVQEVRENAIGEIRNQIHDILIKNNADLIYEYNISNFETGSGRITKIYDSIKKSDVYAENEADKFVIQHVWGIKKSIASHLSAYGSSYTCSNCGRSIFSFYENDICSSKVIKRDGNIITIKTPKGEVFAYSKDKKFNIGYSFSQEKSKEEMKNLFMKIVKAYARPPLLKSEVLLTQKKLDREFLEKFKKERGNSAIFVCPFVDCQSLADSDIQAAFIMALRGYLKKKKGKDINYLEESLNYLQNFKGKINFSNLLN

SEQ ID NO: 124 - protein, 1227 aa, synthetic construct
AIIAFDHVLKLSQWLSKQIESANSPSSSGSTHTYSPKSLFGLENNFNALSNTLGSVLNSLRGVRSKSAHNTHPEEIVKYMIKICRPLEKHKDKLDTIFKTLSQRVKDLEPKFVANWAVYRSFLGGKIQSWFSNFENFFFVDSPDLFTEELGGLKANFSDASNVTVKSSNGSLQTPEPLENQKSERTTAKKDRDKEKLNRSGVVRLCAKKVAVEECMRYLEKIGCLLRECPESVKTKIKVDFEGRINQVLVFLENLHNQIKSTCAFLNRGAALIYFGQDLGLSTEMNVSSPFSEAFSAYEATIQQIKALTPELNQLTQALDGFFVENFSRTTRRRKSNKKTKQVPKELSGFQTSFEANLKKLKELVQEIADELPKVPYFFGEAASRRHLKAIFAPELAKTLFEGVLTWFEQVNEQLETNSNLELYLQRELSLKEKQRILQIFKSSDDFRVFAQKQLKSRFNNQSLANLSLRKAWLQLLHDAKRDPKGQANNNRVYYKSEYSRSHSEVISAPSLNLRQVLNFVLFLKSSLLNSFESVPPTFDIPQKAEEGAVLGQVWTRLQKFVVLIRSVLPMFFETSSDSNDSSISSQENNSECLSPQMSEFQVPRPLLTLMFKMLKDCLYRVGENLSQKFEAIEEILVQPNLLEDLIGIGCKQLITMNFEASAAVGHGGTHAAAPLADEDSLHSLSPFLKDLQTYYFSLIEGLLKVARPRRFLERYVVQFINGGSRESRLAKSKEDEEKYITLLPSVPHFFRKASSPKADNFAQELRVTLFDWLFSWLERLYEKSKQSANFEAYFNRGLTPKEKSRLLKILTNSQGFVDFAEKVLEASRSPELGFQLEDLTGVWLPCFETVRRLNQHGVSHNLQNKSSNEVDLLLSEIDLKLKDVLAFLITLKQAVFKLLIGREKAVEGQSTSSFDEMALLQKFWLVARSSISTILGISGISSSRGKKRRFARRQSSSAEQSFTSPLQTFPVDVFHGILWLLEKSLKEFPSKLKERFKEQDFRSLQNLLAPSFSWVSEDGKLMGDNELDLQSLKEQALQTESTELYLQFLAKYFKVFEARFLVLFEGLLWPAVLSSKGDVVSLIARLKKVDTQGRKNLWDFFVELKKFALGSKNLKGFEEHLEPRIRTSRYQLIFLNRFFNRVLRERWPSSKETEWIEPEIVLNEPSLILERWCFVRPKKFEPPEQFENLKKFGDLYLLDLAEQIDNPILFLAQPFTISVSVKDKQV

SEQ ID NO: 125 - protein, 1184 aa, synthetic construct
MFNQKKGYRLHLERIIYSGGEITRSIKYLLASYSDSQKNKELLNNFSQDLYNDDLKIRGCLNLNDLVNNNQIYNLADFWIDSLRAGVIWQSSASSLIDLIKRLNHQETIGEKIFNNANERIKRFFNSEKFIKEIILSEPKRISSKKQAFYNSLFDILKDEFKKQEKNEKIIIDNKAEQLIKEIVDAFYSNDGVFLMEGEEKQNNFWQEKFNIDKNMIKKEKEDILKDVGDITAFIHPPLIILKGDFSQLIDERKKYFSEKDLEEILGLSDNFNAFSHYFNKFFLLLYQDKQEKIFECYQKIFSFSQEDRKRIKDALDFLSEKSKLLGLPKIVNSWSDYRSVFGGKIKSWFSNYLNREDKAKKQEKKIKEGLEKVNKFLLDFIQKNQVDSDLQQEIKFYYDKLNQFINSYQNQEFFHQQELFLLFSDLLAEYREKLNRFYQKYLSDKEKEEKKVDEFPLFKDLFEKYEGPISFYGKTKLEDNKKIIDLTFKTIKVGLNLIRRLLLDLYNSSDFKNSDNNNQERDLRRIFEFLLNKIPATKTFREKYLSILKDNFDQQTYKEMTLKPSRYTFVENIYSRENRKLIELPSKNFEELLSKIIKDLTDFSLSFKNDDLFVDIYLLSDLVELAKTLISLVINYSNKSQFDSYKNELIDDTYQKAKKYLETFKISFFNSKKEANYFYQTRVLSELKGAVALFSKKYYQAKYNIQILKSNEIFPLFVKFSDLLKKEEINDINKLKLIFKKPYRYLIALKKIKFKKKQQQSSVIHLDKKNKDLVLISPQDEDFLFKLTSSFYQLQFLDRFVYPVKKWLDVDITLSEWSFILEKKYKINWDFNNGKPEFSEIDSRLYLNIPFKIKAINQQKILKPKELFLGIDVGEYGVGYALVNFKDEEIKIIKSGFIRSKNIASIRDKYRLLQDRSKKGVYFSSTNVVQEVRENAIGEIRNQIHDILIKNNADLIYEYNISNFETGSGRITKIYDSIKKSDVYAENEADKSVIQHVWGIKKSIASHLSAYGSSYTCSNCGRSIFSFSENDICSSKVIKKDGNIITIQTPKGEVFAYSKDKKFNIGYSFSQEKNKEEMKNLFMKIVKAYARPPLLKSEVLLTQKKLDREFLEKFKKERGNSAIFVCPFVDCQSLADSDIQAAFIMALRGYLKKKKGKDINYLEESLNYLQNFKGKINFSNLLH

SEQ ID NO: 126 - protein, 1191 aa, synthetic construct
MPPIQPAQKKHPKAVKYLGQTGYTLHASRFSYTGMGVMRSIKFPLYHSERKVYDTFCQQIISDYKLVQGATNLESFLVDEQSYSLIDFWVDSLRAGVVFQPTEAQMSAFLRQHYQMTDVLEIAFSNMDRELRDKLDYLAFRQKIMLSTGLRPTSKFPGFLKCFKPDLQKDEAVIQLANQIFNHLVHDGVALTQSEQKEYWLSNWKLNLPEKSSTGESSTFFILPELQLAQDMTVGQCLDQRRQLFPDQMLTILGFENTFSAFSNYYGKVITELRSGDVENIEKTIKSMSSSWQGNEVELKKRLEFLSARAQLLPVSYLAKYPDYRTSFGGKLQGWYSNYEAQIKRIGEQIGQFGEAIQKAIDDVRVVGVHENDVGFQESHEQRKSVLLTNLDQLKQLQLQLADADNRDFVTLELFRDLLSYVRTDLNWIYQTYLQTDKEKKEEKMVESRYRGLFVKLKLVPEFFGDSRKKQFAKYRDQVVPILRDGQAIVDKLKTELLTSPDYSFVDQQFFVKQLQKLLDKLQTKSWNTKKYQQMVEEVVKKYNNGLMPGRQQAFFLSPYARNKHCQVIQIGVSLDWGSELSDLIKQLNHSFSLDQNPTELQDWLETQKWLIGWLLRANPNLEIDSAEWPTQVFTKARTFQQQFGTKLSGGGRNYFVNSLIFSEMRGAASSFSRREIAMRYVVQPIASLNKYPLVLKTKQENPDLKDKHGLVNQPHRWYLASQKPISKKESTFESEVGLVTEDNDGKRFGLLRSAYFDPGTLLEIRTSKYQIHFLDRLLYAGRKWCDLDVTASDHSLIVEDTYRVNWNLQTGQPGVQKIDENRRLYVSVPVNIKPVLDNDEKKVKKQAIKDGRQRYLGIDVGEYGLAWVVLDFASSLNPRRIAQGFIFNQSLRKIKDKAEEIKDTQVTGTFTVPSTKLARVRENAITSLRNQVHDLVIKYNAKPIYEHSISNFETGSNKTTKIYRSVKVSDVVAENPADDAVKSHVWGSKYITVGNHISSYATSYTCSQCHQSIYDQGLESQSEVKRHVVKEIYGRVIIVQLANNRAVYGYVPPKRDEVCFPYHTGSQLSDKEVYTLVKNFARPPLDKSEAVSFVKNKKPNQLPEKELSLLQESRGNSSIFICPFVNCLHVADADMQAAQNIALRGYLKDQEPESIRKKREKNREAFDYMGAVNMYFKTVKLESMNLEIRT

SEQ ID NO: 127 - protein, 1189 aa, synthetic construct
MAKRVSLSGYRLHVKRLHFASEAPTVRTFKFPLVRKCGKKTDFTDVPQELIEAIKHDDLYIRGPSNLVDYLRFKDRKIYTLIDFWIDSLRAGVIFWQDARRLRTDITRIYSVSFLDEYLKEKSPKLFKYFKPADFLNKVVIKRIDLRKQGKKEKLKKLLISLIRDEYKNNNRSEGELGGEDIVVDLEAKNLVDDLTSNLLKNGQFLPEKDRVSYWLTKWNIYVQELKEITEYDFTALIFPEISLDLRVEVNEENVLEILNQILIQRLIWIAKTPGTVKEDTRSLLLKYSDELRKAVAHPNLLSEILNEAWEDIYKEVYKEEARNSQRTKNDKKDELEEKVIKKTNSEFKKKIVDPVANALKELLGLSNKASALSNYFNEFLEYLKKSNTELIINYYASLGLPKKKLVEIREKLFWLSEKAKQFHKSAGKPKLSKLLGKGINNWHDLRTVFGGRLESWLTNHIERLAKQYRQIVILKKSLPKIIEFTNKYPDDYRDINPDEINNRKQEIQGLIEEIKAKVGNPSVVQDNLEDYEVLEHLVKALRQRVNEYFQLYFRDDLERRNTKLSQVKELKDFWSCRVYSPRSFYGYAQRKRLRKIIETTIPTIKTGISLTRYFLKTLKELPDYTKATQEKDKRKVREDAGIRRLLEILRSKYKDKKLNSDKFKDFYRELLLRYATGKDLASNSKTFYRSEYSTSTQQEIELTVSDYAREVEKIVTEITEFVLSVTDKELLNDAFLLLDWIETARLVVARFFRWMDQEKEWDVTKFRLSTFPAANKFIGRLKNKQVKTPMFRFIIMSFVFSEFRGAATVFSKKYLISKWNVQVVGSDKKFKLALAPKNQDVDIERMYQLIVRRRPEVLKNIRWQVIIYTKQGNSQQLITLKNKSGSFSLSKEKINDTNLLQICTSKYHLQFLERLIYQPQNWQDIEIKISEPSLIVEEVDEILGWDLNSGRPELKKLEKNRPILYYALPMQFAVKRAENTITQLQKIKQAKPEDVYHLGADIGEYGIAWAVIKIENGQVVIRDYGFEYDKQIRKIQDKFNEVQQKARQGVFLEASTALAELRENAIGYLRNKLHNKLIDYGAQIIYEANISGFEVGVGRVTKIYDSVKRADVGNHPDVSTKIYKSLIKHIWGLEAPRAKGDRMRDKSPGWQVGAAGTSYTCICCGQSIYQLGNNKKNKLKEDKDKKDK

SEQ ID NO: 128 - protein, 1187 aa, synthetic construct
MPRSPKRKPAYNGRTGYALHTARLSYVGKEGTRSVKIPLDGHQETVDTFWDHVKGDYIGQVGEMNLADWIDGERSGTHGYSLADFWLDTLRVGGIFSHKEHDMRAIIAHMNGDTSFEKRFVDAVTERHPTLASLADTRALYNFFISGSRNGNKTPDQIFQKVRNSVFPAYRNIEHTEVDAHTRRLVVASSIEGKTGDGDSGKIIYAKELVGFDPTPDIPYANTFFIHPGIPVRFGASAKELLFSYRTFVTQIHPELYGFDGENENDTLKKAFGLFNNQGAFSQYFNAILADLRTGGSTLEHAIKAVSPYWRDHDEDLHVRIAYLAEKAHMLPEPYLAKGWHEYRSTFGGKIESWVSNVLRQEQEIRGQLFGEMKKSKKRNDKKEAFVPGHKQVLESALADAILSDAVKKNINDCALLVSRMETSGVTDADLLLYRAILGDVRIGMNTDYQMAYPVLDGKYEKERREEEKTKRAYRKYAPLYKDIKLIPNFLGDSKRIIYKKFVRSAELLHTGVAAMAYAEDVLATAPFSPRFVSDTDADAFLCKLYDVLRRKYGTLNATRFRGVIEGIFRNLKVSIDGESFNSASALLPDNAERNGITYVCYMSPYARPNPRLKYADIAITTEEIPKVLDTLLVALKPYWNDVMQSGDMGELADAVEMEKIRAGIIVALWYKNTLSTQDILAKLPEELFESAHAFARMKGNPETVSGDDMGRLLQTGILSEMKGAIAKMSRMEYVERYTNQRMNSEEEMPLCKTDTGWHIASEKTNKKELAGLVPVSAKKGKVFSGEGDYVSAFARPDTLFRLNTSAYQLQFLDKTLPHGNRATSWWNAHGVDFSLASYSFIAERPVYIHWNVAGGTFSLERSENKRLFVSIPFSLSPEKKPADVLEHRTRYMGIDVGEYGLAWTIIETDREQKKISRILKQGFIYEPLTHAIRTYVGSLKQQQVKGTFGMPNTKLERLRENAVGSLRNKVHHIAMRYDAIPVYEFEISNFETGSKRTTVIYNSVKRADVGRGGSEADKADANLVWGKNGKNFGKQIGAYATSYICSRCGYSPYEAFQTSGVRETLNDEKKRSRPSLRDFLTTHPAFATHEAFVSRGDKESAWKERRGNSGIYVCQRCGHITDADVQASYWIAAKSFANNLRGKKEQTDADSEKIPPVSFKELLLVHGIDDDHVADNKHITRLNTRL

SEQ ID NO: 129 - protein, 1184 aa, synthetic construct
MFNQKKGYRLHLERIIYSGGEITRSIKYLLAGYSDSQEHKELLNNFSQDLYNDDLKIRGRLNLNDLVNNNQIYNLADFWIDSLRAGVIWQSSASSLIDLIKRLNHQETIGEKIFNNANERIKRFFNSEKFIKEIILSDPKRIGSKKQAFYNSLFDILKDEFKKQEKNEKIILDNQAEQLIKEIIDAFYSNDGTFIMEGEKKQNKFWQEKFNIDKNMIKKEKEDILKDVGDITAFIHPSLVFLKGDISQLIDKRKKYFSEKDLEEILGLSDNFNAFSHYFNKFFLLLYQDKQEKIFQCYQKIFSFSQEDRKRIKDALDFLSEKSKLLGLPKIVNSWSDYRSVFGGKIKSWFSNYLNREDKAKKQEKKIKEGLEKVNKFLLDFIQKNQVDPDLQQEIKFYYDKLNQFINSYQNQEFFHQQEIFLLFSDLLAEYKEKLNRFYQKYLSDKEKEEKKVDEFPLFKDLFEKYEGPISFYGKTKLEDNKKIIDLTFKTIKVGLNLIRRLLLDLYNSFDFKNSDNNNQERDLRRIFEFLLTKNPSVKIFREKYLSILKDNFDQKTYKDIILKPSQYTFEKNIYSRENKKLIELPSKNFEELLSKIIKDLTDFSLSFKNDDLFPDVYLLSDLVELAKTLISLLINYSNKSQFDSYKNELIDDTYQKAKKYLETFKISFFNSKKEVNYFYQTRIFSELKGAVALFSKKYYQAKYNIQILKSNEIFPLFVKFSDLLKKEEINDNNKLKLIFKKPYRYLIALKKIKFKKNQQQSSVLHLDKKNKELVLISSQDEDFLCKLTSSFYQLQFLDRFVYPVKKWLDVDIALSEWSFILEKKYKINWDFNSGKPEFSEINSRLYLNIPFKIKAINQQKILKPKELFLGIDVGEYGVGYALVNFKDEEIKIIKSGFIRSKNIASIRDKYRLLQDRSKKGVYFSSTNVVQEVRENAIGEIRNQIHDILIKNNADLIYEYNISNFETGSGRITKIYDSIKKSDVYAENEADKSVIQHVWGIKKPIASHLSAYGSSYTCSNCGRSIFSFSEDDICSSKVIKRDGNIITIQTPKGEVFAYSKDKKFNVDYSFSQEKSKEEMKNLFMKIVKAYARPPLLKSEVFLAQKKLDKEFLEKFKKERGNSAIFVCPFVDCQSLADADIQAAFIMALRGYLKKKKGKDINYLEESLNYLQNFKGKINFSNLLH

SEQ ID NO: 130 - protein, 1187 aa, synthetic construct
MLPFGAGALIIILNMQKGYNLHKKRIDLLKGEILRTIKYPLKPLNENSEKNKKQFELQIINDDLKIRGDLNLNDYLNYTFKNNHIYNLFDFWIDSLKCGVIWQPSFNNLKKLIEKIYLNKTISEEIFDNSIPEIKKFFDKNKFIENFILKTGIRENRSKKSFEKDLIKSLNDEFNKEEINKEAQDLIDKIINTFFNENKLKKLTIEEQNNLWLNKFNIDKSKLKTKKLKYNYTFFIIPELINFEENNLENLLNIRCEFIKNKNLNIKILQEILGLENKFNSFSNYFNKIIKNLQNDKYNLIYEDFISFYKFTNEEKEKILQSLKYLSEKSKQLGSPQILNLSWADYRSILGGKIQSWFTNYLQRKEKLEEQLNRLKENINKTKEYLLKNFQKNTDLKEIIYFLDKLLELSEVSKINNLFFDIHSTCKLKINNFFQKNIKQNEDDTIESNENLKGIFEKIYKPLNFYGQSRKENNIKIINSLQTVELGIIYTKKILNFLKNDLDFNKEENFEEIYRKNLNFLWNKLKENSLNSKNFKSKYKEIIIKNLNSYSNIKFEKIIKRPSYYVFYKSIYSKGTNLQIEFNKEINFKNIFIENINELYNFLINLEDKEIIIDKNLLLDWVELSKNISSYFIKFTNIEKINLKEIFKDKDLNFFSNIAKYFETFGNVVEKQGLSYFIQSIFFSEFKGAATLYTKNKYLAKYNIQIIDSNKKFPLYYYSNNLKNHNKLLNPKDLIKEKHNYFVSLKKIKSKNKDLEFLLLNKDNFEKIYNSFEDNKNDLLKLESSFYQIQFLEKFIYKPKNWKNSKIKLSEWIFILEKEYEIKWDLENKIPILNEIQNSKKNKLYIAIPFKISSDQKEYEEIKKSKENYILGVDVGEYGIGWVLINYKNKELKIINKGFFVSKNIAKIKDEFSKIQKKSQKGIFNESSNLISEIRKNAIGELRNKIHSVFIFYHSKNICYEYSINAFETGSGKTTTIYKSIKKSDTLSGIDADKKEQKHIWGKELNKIEIGKNLSSYASSYICSKCGKSLYQLKETDLENNEIKIIEREGNILKIKTPYGNVYAYSSKKEHQVDFIFNKTKSSFKELQKIIKDFARPPLNNNSEVIRKFTNLKENEIDILHQKRGSSCVFVCPFCLHVSDADIQAALMMSIRGVINNETKNKGEKIDYFQKNLEFLNELKEKPVIDL

SEQ ID NO: 131 - protein, 1227 aa, synthetic construct
MANRQISPVDSTNNFIFTLYVSFDTVLYARILAFLTLSCLLVGFLEYIYPLGVIYLIMPTNQQYDGLTGYQLHTERLRHTGKSGIRTFKLPLKTNDQELFGNFFENIKHDHEGQIGATNITTWTEEQKSSRQIYSLLDFWLDSVRAGVVFASSSAELNELLSVCGDEDAVDEQVKTAMRAINPTFFKHLKFNDFKEEIALKEGMRSSSTKNSVTRRLYKCLDCTEEDAPEEIQQAVADLIKTFFTTDGKIISRNDQDDYWQVHFGLDKSLYKLDSDIKLTFSFLPDIPFTPEADAHKCLDKYRCWIDENAEKFQLESKEDKTNFLQIHWGIEGNYNAFSNYFNQIIDLLREEEGRQKLLDALLNTSDLWEGAETELQKRFDFLVDKAKQLPKAKCVDSWSDYRTTFGGRLSSWLSNTLNQEEFIKDTVSDQKEELNQKEELKQIIKNSEINLYKKAFSAADDNRAVELSHEAVIAQKTLEKLQKSSSFEPQLLGVYRDNLGRLRSLLNKAHQLLPDEIENKSAHEVYSALHERIRLMPKFIGGAKAARFEKYVKSLQILKQGASFLENFEEKVKEAMKSSVSVEVEEISEGYFLRQLNTLKRFYDNANDSRFKGKLSSLFDKDLGVNIEAVSNRETFYISPYSKHDNRAVISVEVANYQQLLKSWVNELKPYWGNIIATENWGEIIDAMQLERIRIGWIYKLYPKLTFAISDDLDELFAKAATYRDLHGHEVEGSLAANFLQRIILSEIEGRVKEASREKFTVRYVIQPVGSEKTTKYPLFVTSNKDKLSATERKQWYIRDINSSFSRSETDKKGWILKNGKDFSNGFQEKYISDNTDLFKIISSKYQLQFLDKALPNGAKKFGKDGDVNLKLSEHSFIAEETLQASWDFSTDTVSLKSVDKDLFVAIPVNIEPESQRSSREERTRYLGIDVGEYGLAYTLIETIPKVRVIKSGFLYEKTLRNIRKQVTNIKNSQRLGTFGVPSTKLARLRTQAITKLRNKVHDLVVRYDAIPVYEWQISNFETKSGRVTKIYNSVKRADTNSGANENADGDEAALVWGKRPYKIGQEVSARATSNMCSNCFKSIYQFTEQDFTEESRSGEYVEFKIAKNTVKGFVPNDEKITSKQKMINAVHDYARPSVDILVKRNDVGVDKEILVNFLEHRGASALYECPFCSHLSDADVQASLFIGLRAICRDQDDETTKVKISDLMDFAQKQNIDPISADFIQ

SEQ ID NO: 132 - protein, 1262 aa, synthetic construct
MGNVQSTRAEQNATAPSRQPKIKHPPSEKGYTLHDRRIAAAGGDKAIRSIKYEVVFADQDRQRVIEAIRADHLAINGPVNFRDFVRHEIEGTPRKVSFVEAWFAMLRAGAVFQPATGALTNLAARVRGGSPLKKFRDTFRPLLIAEYAELIDWAELFDFVSKGTRDTTNGQMGGKILRCFSADAKESTDPATVAIIKRAMDSFERVYPLIRVFKGDGEAKKRHELRQKIAIFLKEPPPAEDGRGAASVAIQDSPTVLLARGPGVPAAASRMITATAIEKTLSEIEGWASALSAGDVEEEAANALSLSTLLGLGGAGFSSLANFGNDFLPGLADQTKLPNLAATQAALLGQDAAGLAQRLAAVQASLGDVDWNMQLANGPGDYRTDIGGKLQSWLSNRDRCRKELATALDELLVELQDKAAVLLEHYSDATKVGQEVARWAARTTVVKAAVTGLTTRIPLAKGAIEQNQENAQELLDLLADDIAALNQELNEWFQVVVESRRRQGPPAGRRSRSPGGRRGEASFSVEWFESRPADELDFRKIFKQLSKRLQRIPLFLGLQQQRTYVKAYWAPRLVRVLHNFLLADLKALKEGATPSALSAPEKQRVLAWWRKVRDQCIGAEQARFARMVGAAAPLHPFQKVPDDAVIYKNPRSRARVRVLAESDHPALGSHDIIDWLLENLSLGLPALTQHGWDLYLALVELAKSFIPILLERSSAIDRRIDAARLAVDVEGRWWGPAPFEKLKVALALRGGETGALSTQDYQYLVNLLCTELRGLVGILSRKEITQNYTLQSINGSQSDLLVAAAQAVDVAPDKARFMLHLPRLLRGAAAEPTPPLLRWIKGNESTVIVPTAPRGVVGEIRSSKYQVQFLRWALAKNLEKRWGKKRCQLEVSGTSLVLEERVPLKLDWSTERLTPLHDQVTSRIFSVVPFSFIPPTSQEGLLPISSNRFIGVDVGEFALCYSGIEVAGDKGSADVKVLGSGVIFDPQLRKLSKRVRELREAQRGGNFSYRSTRVARLRESLISSLRSRLHAVLLRYGATLVFEFDISAFEVGGNRIAKVYDSVKRTDTIPDNDAEKAERKHCWGRTNFRSGAEVGARGTSQTCTKCGRWAKAHVKEHLSGPAIKASALVREDSNVYAAKVGAASLRLFLKQPPGPEALLDEGALTTAIHDYMRPPLAQASAQIGDPVAAAAIRGNSSFFRCPFADCNHVADADLQAAFNIAMKGACAQLWKHKGDDWLLQFAAEGRPAVGIDSKDYAWPKGS

SEQ ID NO: 133 - protein, 1195 aa, synthetic construct
MQKARNGSNGWKTSSRHARTGYRLQLARLQRTGKEGVRSFKFPLMPYGGTGSELSAFAKSVEADYRNSVGLLNLSDWYENEKSGGGHYSLVDFWLDSLRMGVIFAHKKVEMKNTLLLLNGDTSLRKRVLDAVWRRDARLYGIADKENLYQFFISPSRPGKKTKEVFAKKLNGDLFPSVPESDVAHASEYVSRFSDWVFSGTKQAQVTALLGFDPKANHSYPYMFFFSPHLSRQEEASPKQLLATYHSYLRDDRSPYREHDQEEQIKRVLGLYNNHGALCNYFNKALKKLQSKDSGAIVQAVRAASPYWSEHERELCTRVEYLARAAQKLPKATLDERGWHQYCSSFGGKLESWVSNTIRQERLLRAELFSGEAAGEMSTEQDAQRFKKKKRKEKEEKARSSHEAALSAALADPILKEETRELVRQCQSALTQLRTEISDDLLAAYRLLLGELRTALMQDFQLATFDDSEPGEEIKKLQAPYKPLFARVKLIPNFMGDTKRSVYEKFIRAPRQIKEGIRCVTACADTLKQSACDPRISDAELVGHARKVYETLRRKYPALNATRYKHIVESLFAARRGVLEDGTPFALSRLLASADDRRNRLDANRFVLWKSTFNRSQAARVTLDISADAAPAILKQLAVALAPRWDDIVVTNDFGELLDACEMEKIRLGILVTLHHKTAVSIDIDALSPDLFGSAHAYFSFLGKPASLAGETLGRFLQAFILSELRGALSKMSRTEHVERYVVQTLRSEEHFPLCLREHAGKRQWCIAAERPAVLRDRHTQCPEGKVAVQLKNGKDFGNKNEFAAACADPTSLLAIRTSVYHLQFLDKALGGSSSWWSLRNFNLKMGEPSFIVERTVRTTWDIVKEQVSFERDEPRVYASIPFTVQGATHTAPLDERPRYMGIDVGEYGLAWAIVERGSGAKVGKIVASGFLFEPLTHEVRNFVQTLKAKQSLGTFGVPTTKLERLRENAVTSLRNQVHDIALRYRATPVYEEEISNFESGGNRVKVIYNSVKRADVERSYDDDAGTMEADLVWGKKAKQFGKQVRAHATSYICTKCGRSPYEEQDKAQKEKMRGDSRPSVKDFKNLESKQFTDWQQHPLLRQNSEGVSPWEERRGNSALYVCQYCNHVSDADRQAAYWIALKRILRDKDKRDKDKKADADKEATNEKKFDLKKDVAALAAAHRKSGNPPVELKL

SEQ ID NO: 134 - protein, 1176 aa, synthetic construct
MIISKKSKKHLGQTGYTLHSHRLSHTGRSAMRSIKFPLFHTDGFASISGTGHKLITSIKSDSELVQGIGSIDSYIADTKAYSLINFWEDSLRAGVVFQPTEATLQDFLGKFSGSQDENQLIFKNFRYDFKYALDFEQFKKKILLSVGLRSTSKKSSLINCFKPEHRKDHRATQWVDTIFNDLFKDGKSMSGDAQIIYWREKWGLEKKNASSSSIKLTFTILPIFAFSATKTLEECLSERRKLLSKESSYDINSILGFANTFSAFSNYYGEVVSAFRTDNFEGIYNALCDQPLGWLKKQKDLRERLNYLSLQAKKLKEPRLATSYAAYRTSFGGKIQSWFSNYDSQLEKIYKQLHDHAQDLAQAQKDLSQIEIEENEAIEQRYYFLHDSLAQLTLLQKKLTGQKSMNITLIDTYNDLLSLVRGDLNWFYQHYLQTEVEIKQEKNIENRYKYLFSKLRIVPDFFGHSLAEKSNKYICKTIPQIEGGLLMISVLKQKLLDTSNFSFADEIFIRQQLNTLLRKVTTKSINTKKYIVIFEKALEVFSGRKSFNNDEVFYISQYSRQKNLKIIQLSASISKDSLLHLISILDNPFLLNQDNSAELVDYLETQKIITSWMCRGYTEAEIDISGLDFENFKKAKTYLVQFGSKIPKNRLSYFLSTTIFAELRGSASQFSRKYILSRAVVQPMETLRNYKLLLQTAMDSSCSRLDIVQNPHRWYISTDKQLNTGDAVSKNFIMSGENSGKKFSTLKEGNYNPESLFAIQSSKYQIQFLDRLIYQSRKWHDVDLLASDFSFILEQTCEIQWSLTLKRPTLKIKNELTKLYASIPFTISPIVDKKELDEKKSFLKSRKKYLGLDVGEYGLAWVIMDFTQSKTPVRIKSGFIFDGALRKIKDRANEIKDLQMKGTFSVPYTKLARVREQAITSIRNKVHDLVLKHGAKPIYEFSISNFETGSNKTTKIYRSVKTSDVSTSNDTDADRAVASHVWGKNAVKIGNHISAYATSYTCSECWQTLYELDLSGKKEKTLEKVRQVEKNIYCATVNDKQVYAYLPGISSKNLEKILSEELLKGIRSFARPPVEINEALSFAIKSSKKSISKELLSHFKSKRGNSALFICPFCLHISDADIQAAQNIAIRGYLKDVQLLSKPTEKTNDFNYISEVKKFSQNSKLPSVDLEDFEYS

SEQ ID NO: 135 - protein, 1338 aa, synthetic construct
MDKKSSNSTGYRLHKDRILFSGGEIMRTIKYPLVVEKNNLNSEEIVEKIRQAIINDDRVIRSDINLNDYIEYTKKGNRLYTLIDFWQDCLRAGVIWQPSTSFLLYLINKLYSKPKAIELIENAKPDISRFFDVDKFSKCFILPGEIREGKILKTFKRELIEALKGEFKKGKKEKIKDEDDYLEKFVEKDARKLIREIADCFFSNDILVTHDLKEGKKEYQDRLWEEKFGIKKGKLLENFKLPDHLRNFKNISFFIIPELSDKSKNFDELIELRRKWLLERKICVREDGDYLENEKKLDEELRNLVGLSDNCNPLSNFLGTVFCELLVPNNLNEDNALEKFYDVFTIVEPKIAELNIKDQIMGSLEFLRLRAKQLGSPNLVNFSKSQNLKANESIKLDGWSLYRQNFGSKMQSWFTSYIERNKLLEDSLKNFKEKIKKAQNFIKNLKNISEEPQQEEEAQQEKEEIVELFEKIFSSLEKVNRENFEVFDSLLSSLRKRLNFFYQQYLYNEAKEGDDVKKHKILGPIFKNIEKPIAFYGETQRKKNEKIVKDTIPILEEGTVFLTTLISNLLDSFSPKQVFPDVRKKDETEEIIYRKELQFFWNKLKDLAVNSKEFEKEYQDIIESAVDESELSKLKELFVNKKKNGSKYNKYTFYKSKYTKGSIEEIKLKGSKEEYLLRFEKLIKSLTNFLTQFNRNKLLQDKDLLLDWVELAKNIVSVLIRFSTNTEFSLNEIKAQSQFKKAKNYLELFKLKKAKKKEFGFIIQSFILSEIKGAATLYSKRKYIASYSVQIVGSNNKFKLFYQPLDSSINISGGPKDFVTKKHKYLIVFQDLKNVKNKDATENRINLLRLNKERKIPLVAYKDDLVSKSLLLSSSPYQLQFLDKYLYRPRGWENIDIKLNEWSFVVEEAYDIEWDLNSKTPKLIPSPKSNRNKLYLAIPFTLKGNVKEPPLDKIVLKSETKKDHSRDKNRLNYPILGVDVGEYGVAWCLTKFDYNQDFSLRDIDIQGKGFIEDRNIGKIKDYFAEIQQKSRKGAYDEDDTTIAKVRENAIGKLRNAIHSILTGSLEGASPVYEDAISNFETGSGKTIKIYNSVKRADTEFKSEADKAEHSLVWGKKDRNQETKYIGRNVSAYASSYTCVNCLHTLFKVKKEDLSNIKILEKDGRIVTMSSPYGPDKKVRGYLSEKEKYEIGYQFKESEEDLKAFRKIVRDFARPPVNKNSEVLEKYAKEILAGNKIEEFRKKRGNSAIFVCPFCQFKADADIQAAFMMAMRGYLRFSGIVPSKENSKNNPQESEDKSLKNSKKQSETGDTFLTKTAEYLQQLRFEIKEKIKEAVKVDF

SEQ ID NO: 136 - protein, 1162 aa, synthetic construct
MSDQGSAQYDGLTGYRLQTKRLGYTGKSGIRTFKLPLKTDDRELFDTFFADIRHDHERQLGATNITSWLDSKRNDRDCYNLLDFWLDSLRAGVVFAPKASALNQLLAQLSGSSVDERVKESMRAANSAFLDALDWNTFREEIALKGAKRSTATPDSFRRDLIGCLQESEEKTPDTVIADVEAIVAQFFTDSGDTIEGKKQKQYWQEKFGLDKSLYDLGGDTALTFYPVADIEFTPEDEAPDILQKYRQWVSNNADTFSLESPEAKQEFLRRRLGNLQESGNYNAFSNYFNEIINTLQDEGGLDKLTQDLLAVSNMWSDQKEELRNRLRFLQERARRLGQPKCTKSWADYRSVLGGRLASWLSNTFRQEQFINETIKKQKAELADLQNECANGVFVDIFSADDDDRDDGLENYLNTARDLLDDLQGQYGFKPEVLKVYRDTLGRLRSLLNRASQISKQKDFTADSYPGLYERVRLIPRFVGAAKRERFQKYRRSLITVKKGIDYLQSVSVAIDSAEADIDQGYFLRQLNALKRFYTQANSERFRHIVADLFTAVGVDADALGADDTFYISPYSNSTRGRNLIEINDLPDTPTATLQAWSDKTAPYWEDILQTQNWGELMDAARLDRIRWGWITKILGDKLQVSLIDNLDTDFPQTKVLRNLYGETVSGQTAGDFLQRVILSEMGGRIKEASREQFTVRYVVQPMKTNEQYPLWVTADDDGLTAQSRDQWYVRYFQQDFSGDESDQSGWTIGAKGKKPQEGFEQAFLPDDADVFAINTSKYQLQFLDNVLPGGAKRFRDDGGFSISFSEHSFIAEETMQADWDFTADRVELESVDKDIYTAVPVQIDPKESASARDQRSRYIGIDVGEYGLAYTLLETEPELKVVKTGFIHEPTLRNIRTQMTNIKESQRKGTFGIPSTKLARIRSQAVTKLRSRIHDLVVRYDAKPVYEWQIQHFETGSGRVTKIYNSVKRADVSVTVQDDADSAEAELVWGSNTWRVGMDINAYATSNMCSSCYRSIYAYESPSFSLIDTDEDYAQYEIETDVSAWAHLPSPDPETADSDEIIRGIKEFSRPGIDVVANRSMNSCDFDHKQVQKLVGKRGNSCLFTCPFCNHVADADVQASLFIALRGLLRDKKSDEESVDVEELLASAQQEGLQVAGDIGS

SEQ ID NO: 137 - protein, 1338 aa, synthetic construct
MNKKSSNSTGYRLHKDRILFSGGEIMRTIKYPLVVEKNNLNSEEIVEKIRQAIINDDRVIRSDINLNDYIEYTKKGNRLYTLIDFWQDCLRAGVIWQPSTSFLLYLINKLYSKPKAIELIENAKPDISRFFDVDKFSKCFILPGEIREGKILKTFKRELIEALKGEFKKGKKEKIKDEDDYLEKFVEKDARKLIREIADCFFSNDILVTHDLKEGKKEYQDRLWEEKFGIKKGKLLENFKLPDHLRNFKNISFFIIPELSDKSKNFDELIELRRKWLLERKICVREDGDYLENEKKLDEELRNLVGLSDNCNPLSNFLGTVFCELLVPNNLNEDNALEKFYDVFTIVEPKIAELNIKDQIMGSLEFLRLRAKQLGSPNLVNFSKSQNLKANESIKLDGWSLYRQNFGSKMQSWFTSYIERNKLLEDSLKNFKEKIKKAQNFIKNLKNISEEPQQEEEAQQEKEEIVELFEKIFSSLEKVNRENFEVFDSLLSSLRKRLNFFYQQYLYNEAKEGDDVKKHKILGPIFKNIEKPIAFYGETQRKKNEKIVKDTIPILEEGTVFLTTLISNLLDSFSPKQVFPDVRKKDETEEIIYRKELQFFWNKLKDLAVNSKEFEKEYQDIIESAVDESELSKLKELFVNKKKNGSKYNKYTFYKSKYTKGSIEEIKLKGSKEEYLLRFEKLIKSLTNFLTQFNRNKLLQDKDLLLDWVELAKNIVSVLIRFSTNTEFSLNEIKAQSQFKKAKNYLELFKLKKAKKKEFGFIIQSFILSEIKGAATLYSKRKYIASYSVQIVGSNNKFKLFYQPLDSSINISGGPKDFVTKKHKYLIVFQDLKNVKNKDATENRINLLRLNKERKIPLVAYKDDLVSKSLLLSSSPYQLQFLDKYLYRPRGWENIDIKLNEWSFVVEEAYDIEWDLNSKTPKLIPSPKSNRNKLYLAIPFTLKGNVKEPPLDKIVLKSETKKDHSRDKNRLNYPILGVDVGEYGVAWCLTKFDYNQDFSLRDIDIQGKGFIEDRNIGKIKDYFAEIQQKSRKGAYDEDDTTIAKVRENAIGKLRNAIHSILTGSLEGASPVYEDAISNFETGSGKTIKIYNSVKRADTEFKSEADKAEHSLVWGKKDRNQETKYIGRNVSAYASSYTCVNCLHTLFKVKKEDLSNIKILEKDGRIVTMSSPYGPDKKVRGYLSEKEKYEIGYQFKESEEDLKAFRKIVRDFARPPVNKNSEVLEKYAKEILAGNKIEEFRKKRGNSAIFVCPFCQFKADADIQAAFMMAMRGYLRFSGIVPSKENSKNNPQESEDKSLKNSKKQSETGDTFLTKTAEYLQQLRFEIKEKIKEAVKVDF

SEQ ID NO: 138 - protein, 1278 aa, synthetic construct
VQGRAQYMDKKAKKIVGYKLHKNRIIFSGGNIIRTIKYPLVTDEKSKQFLEDFKAKVISDDRVVRGDVNLNDYLNYSYQGNGFYTLFDFWLDSLRAGVVWAPKSVKLKEYSQVFFGLKSNLDELWNGVNDEIKRVFDKEKFLNNFILKKEIRSTATRNSFQKKLKDSLIKDVKTDDEEVENLINSLVSKFFDSNGHIKLNGRDEQNKAWKCFGIDKEKIFSKKADNQSRNSQLGDITFLIIPKLISFNKEENEEKIEDLVEKRRDWLKNLRIVKKQQEDYLNNILGISENFNGFSNYFGSVLRFLKKGKTEEVVTALMSYLPQDDYFRNNIRNALEFLSQKAKKIPNVSMPKLVSGWADYRSVFGGKIQSWFSNFNKRQSELEKKLDKFKKSLSDTKKFFLKDERIKSKEETKTAVIFIERLESFSNKFIKSEKGFKVFETLLSALKKQLNLLYQQYLFDEKKEGDNVRKDRVLGALYQKIDKPIAFYAETQKSRNKKFIEDTISILEEGIDFLEKLIRDFVGTFAVNYAFMGEKRKDETEESVYRKQLQFLWNKWKDAEINSSEFKNEYKKLLTSVLGEKEAKDIFNRKNLGRYVFYKSKYAKGNVIEIKLSNGDEKNYLQEFDNLVKHLTKFLLSFSKDKLISNEKLLLDWLELGKNIISNLIRFNQKTEFDLTSIKSLVKFEKARNYIECFKIEKIEKKEYGFIIQSLLLAELRGAATLYSKKSYIASYSVQVIGSDNKFKLYYRPLENIGSLEEMLNKPKTERKDLVRIRHRYLISPFGFRKIGNKNFPENTLETIRLGKKKAEVETLFLNDLNSRAFYLQSSYYQLQFLDKYLYASKDWQNVDVELNEWSFAVEEAYRIKWNLDQKRPVFERDLKNRHNKLYLAIPFSLKSRGERDLFLLNKMLKSQGEKDKQRLERDKNRLNYPILGVDVGEYGLAWALVKFNLDPDLQGMSILGRGFIEDKNVGKIKDYYVELQQRSRLGTYDSYDTTIVRVRENAIGKLRNYLHAVLLNLDEGGTVVYEDSISNFETGSGKTVRIYNSVKRADTEFEAKADKSAHEQVWGKGTKYVGRALSAYASSYTCVNCCRSIYQFKAGDQDKILAERKEGNIVFLKTPLGEALAYSKNKLSAKSGKNRFKEMMKILKDFARPPLEKSEVIRKYVFEKGIISEAKINSLKRKRGNSAVFVCPFCQFVADADIQAALMMAIRGYLRFSGLVSSLAKTEDKSLEVKDSLSETGNSFLKHTIFTLEGIPAEDKTQIKETVKLQNTILLAN

SEQ ID NO: 139 - protein, 1192 aa, synthetic construct
MAESKQMQCRKCGASMKYEVIGLGKKSCRYMCPDCGNHTSARKIQNKKKRDKKYGSASKAQSQRIAVAGALYPDKKVQTIKTYKYPADLNGEVHDSGVAEKIAQAIQEDEIGLLGPSSEYACWIASQKQSEPYSVVDFWFDAVCAGGVFAYSGARLLSTVLQLSGEESVLRAALASSPFVDDINLAQAEKFLAVSRRTGQDKLGKRIGECFAEGRLEALGIKDRMREFVQAIDVAQTAGQRFAAKLKIFGISQMPEAKQWNNDSGLTVCILPDYYVPEENRADQLVVLLRRLREIAYCMGIEDEAGFEHLGIDPGALSNFSNGNPKRGFLGRLLNNDIIALANNMSAMTPYWEGRKGELIERLAWLKHRAEGLYLKEPHFGNSWADHRSRIFSRIAGWLSGCAGKLKIAKDQISGVRTDLFLLKRLLDAVPQSAPSPDFIASISALDRFLEAAESSQDPAEQVRALYAFHLNAPAVRSIANKAVQRSDSQEWLIKELDAVDHLEFNKAFPFFSDTGKKKKKGANSNGAPSEEEYTETESIQQPEDAEQEVNGQEGNGASKNQKKFQRIPRFFGEGSRSEYRILTEAPQYFDMFCNNMRAIFMQLESQPRKAPRDFKCFLKNRLQKLYKQTFLNARSNKCRALLESVLISWGEFYTYGANEKKFRLRHEASERSSDPDYVVQQALEIARRLFLFGFEWRDCSAGERVDLVEIHKKAISFLLAITQAEVSVGSYNWLGNSTVSRYLSVAGTDTLYGTQLEEFLNATVLSQMRGLAIRLSSQELKDGFDVQLESSCQDNLQHLLVYRASRDLAACKRATCPAELDPKILVLPAGAFIASVMKMIERGDEPLAGAYLRHRPHSFGWQIRVRGVAEVGMDQGTALAFQKPTESEPFKIKPFSAQYGPVLWLNSSSYSQSQYLDGFLSQPKNWSMRVLPQAGSVRVEQRVALIWNLQAGKMRLERSGARAFFMPVPFSFRPSGSGDEAVLAPNRYLGLFPHSGGIEYAVVDVLDSAGFKILERGTIAVNGFSQKRGERQEEAHREKQRRGISDIGRKKPVQAEVDAANELHRKYTDVATRLGCRIVVQWAPQPKPGTAPTAQTVYARAVRTEAPRSGNQEDHARMKSSWGYTWSTYWEKRKPEDILGISTQVYWTGGIGESCPAVAVALLGHIRATSTQTEWEKEEVVFGRLKKFFPS

SEQ ID NO: 140 - protein, 1224 aa, synthetic construct
MTKDCKTGYKLHLQRLIFSGGEVTRSVKFPLKTIHDSSAPLKECFENKIIADDLKLRGGINLPDYQQNPNNYGLAQFWIDSLRAGVIWQPTASKLTDFVRRYFKEPSKGDEIFAKAARVNTKLRFFEKETFVETIILKGPKRSGGSSGGFRNELIGTVKMEYKEGKNKLSPQAQPVIDQILNDFYDQNGKLKLDANKQNEYWLKEFDIDKSLLEKEQSGAANKELAKLTFFIIPQLVNLDKELTPSQLLEQRKSLFLNPDADSLIEDGKLAELLGLKNNFNALSHYLNYAFQLLYSDKTDEIFQAFTERAPTIAPQEKEVKEALEFLAHKAKKLGSPQTGRGWHEYRSVFGGKIQSWFSNYQRREKELEKQTQKLQQGIKNAQILIDERVRDDSLLKQEAQAVTDLLNQISHVLGKNTDNLQESKEYEVFLDLLAQARRELNFFYQKYLKEEDDETAINKHKELKYFYSAVYKPMTFFAQKRLEQNEKTLRKTIAIIEEGLQIAYRFLDDNQANFDPSSTFEKVKRKNETPEFLYRRSLQRIWGRLKDKRINSAEFRQKYSQILKKALEDESRWNKICADPNRYCFYKSPYAKGTSTEIALKSAEFLAEFRSLVEDLYGFAVSLKDTSIFDNEGQLQDKNELLKTLLADLVRFSTKERFDIAGMDLSQFEKAQKYLELLNRDKLDKNEAAYFYQSLIFSELRGAITVFTNKKYKSRYVLQLMRSPNKFPLFYYSRIKALDAKALKLNRKDIRGQDLLSQNPHYYCIASSQKVEPAKKVQSSPFLRLLRKGTEIVGLEEADHKYLLKISTSPYQLQFLERFVSPIGDWKKVDITLSEWSFIVEKNYAVDWNRKTKQPTFTQNGETKLFVSIPFQISAEKLKEEAGLTNLSAEHLAHPILGIDVGEYGVGWVLAKFSDGIADILRRGFIRDRNIAAIQDNFASIQARAKKGVFNASSNTVAQVRENAIGAVRNQIHDILTQETASVVYEYSISNFETGGGRVTRVYNSVKRADVYPETGADKLEHAHVWGDKYANPGTHLSAYASSYTCASCARSIYKFNQKEIGQARIIAREGNILTIETPKGKVCGYGRDKKFREGYHFKNSKEGFKEFMKLVKDFGRPPVSTRSETLSRFYAHLSKKQLDQFAQQRGNSAIFVCPFVDCHNVADADIQAAFMMAIRGYLNWSEQVKTDEKEKKDFLGKTLEFLHSLKNRHQLSKEFSVSADVS

SEQ ID NO: 141 - protein, 1268 aa, synthetic construct
RTFKYPLVKTCKKHGKQKATNFLDVPHDLIEAIKHDDLYIRGPSNLVDYIKYKDAKIYTLIDFWLDSLRAGVVFWPDNKSMQMDLMQINDFNLYEKFLHNAPMILRIFEPATFVKEILFLDIRGGNKKSKLREKLEKSVKNNINNNRELALQAKELINKISGALIKGQNLLAPKDRKVYWLSEWNIDIDKIKNFFNLNITAIPHPDVTSFMENELTPENIISIINSIIISHIEFAFRYPNLLPTTLKNIFEKIKIENKEFFTKDLKIKVSLNEILLKIKSLATNKTNKRGNSEEKSSRKIIEDLTDKYLKKTFGLSKKAAGLSNYLNKFLRYLKQQKTKKLLDYYKDKLGFNQKSQLKTIELRLIWLSKRADKFLSAADKPKVFGVKGWHDYRQIINGKIASWLTNHIKRLSEQTEQLKNLIKSFNKIVEFINKYPKEEQKYNMQSNPAHKENILKLIKQIRELAKNVSQVQENIKQYKDVASLVKALRQELNEYSQLYYRNQEGQKVKPSEISYLKDFWDLRIYKPRSFYGYAQREKLEKIARSTIPTIKSGINLVQGLLKQLQNLPNYANAEKETDKNSIRKSAGFRRLLEALARKIRDHKINTKPFRDKYLQILQNYANSYELKNLNKYVFFKSNYSTSTQHEIELKNINYADEAKSVVTNLIEFLSSFTQKDLLKDPQLLLDWVETAKLVIARMFKWMDQQKEWDLNNFNLEVFQATKDFIKSLKSNDVKTRMFRYVIINLMFSELRGASIMFSRKLIKTKWNISFVDSLSKYKLMAISQKDNNKIQLSQNLPQRLKWYVIIPKKLAKSIKKLEQPLLYFTKISTKKIKGENIVYERFIKLGKDKLGYRIIQVPNIINNMTLQICTSKYQLQFLERLVYQPKNWQNIIIHISNPSLIVEETHEISWEHNKNKITLSKIQTTNSPILYYALPLQLIVNRPNKKNTPLEAIKKRLHKIKTISNDNKRQKAWYELNNDILYYLGADIGEYGIAWVITKINEKNNIISVEEYGFEFDNQIRKIQDKFNEIQQKARKGVFLEASTELAKLRENAIGYLRNKLHDKLIKYKGHIIYEANISGFESGVGRTTKIYDSVKRADIGNHPDVTKDIGKQLANHIWGLKKGNKENSPGWQVSATGTSYTCINCGQTIYGLGREKDNWERTGTIAGNIHKFKHPIQNITILGYKENFKGQQIDYKTLRKIAKNFARPPLVSIDKNTGKLKPSAVSEQFIINKDVLTEKQLEVVKKKRGNSAIFVCPFEGCHAVTDA

SEQ ID NO: 142 - protein, 1211 aa, synthetic construct
VHTPKVSGIKAFQVVFNSESIGDKDMNLVHSTPKVSGIKAIRHSGNSESIGDKGFPVERLLPSTLFRFYWYKVMSKKLTGYTLHNKRLEYTGKSAIRSIKYPLKPPTQEELDSFSEKIVWDYEHLVGPLNISSYNKNPNMYSLVDFWIDSLRAGVIWQSKSNLLINLVRKLERDILPTEKIFEQIDFKLKSKLDREKVIATIILNTGIRSLSTHKSLRNRFLKCFKEEFWDNTDVLACVDRWSQALIVKGKSIPSKEQRLYWEKEFNIKISSFSEGVKLSPKFTFYINPSLEFNPNLSLDKCLERVQNIEDNPESLLGLDNNFSAFSNYFNEVFAILYKGEVKNLVDTVVEMCPKWSDKKEALEERLKFLSRRAKELGLPKLSNSWADYRMVIGGKLKSWFSNYRGQLEKIKEDLEEHREALKTLRKDIEKIEVIKDDPFTKTMKGDLTEQRVVLLKLLETLLVGDKIAIDDFELYQFLLADFKGLLNTAYQKSIQTEKEKKEDKDVSKKYNALYKKLHNMSRFFGDAKKERYQKIVNSSLPAIDIGLKILADILEKLETTKEQDFSLITEEYVKKQLEKLNKKYKSRAFNYNKFNQITEQILKKYNNGNLPKFNEVFYKHPREKSINNKELPIEISNPPETFRYLIDKYKIDLDWKTLNPNEVADFVEVYKFTLGWLLTLNKSLKVDFSRYDMPLFPKASAFIKRFGGELEGYSLNKVIFSFIASEIKGFVTLYTRETFVVRYVIQTIASEKKFPLLCIQEDKKEETQDFSRKWGVLIRGEEPKRESKIKCLKIVGKKNVTSAKGEYVSPDFIWHIKTSKYQIQFLNRLFCKTKKWNSMDIELSEPCLILEEKWKILWDKEKMKPVFEKEENDQKVYYSMPFDLKPVANQKQRKQRAEEIENRTAYLGLDVGEFGVAYVAMKVDDNKIKILEKGFITDPALRKIRTHIQSFKERQKAATFANPSTKIARVREMAIHSLRNQIHNLALKYNAKIIYEISISNFETGGNKIAKIYRSLKVSDVYPENGADRAVVKAVWGERSKLMGNHISSYATSYTCIKCFRTPFELDLDKKYSFSKEGGKVVVDINNTKVSGFVRKSTDNDSKSNKLEVKGKVALGWIKDYARPPISEVLLPESNLEQKLHNRGNSYIYRCPFCGYVTDADIQAAFNIACRGYISEIKKGKKGKGFEYIKEVKTLWSKNNNPWKEFEFPI

SEQ ID NO: 143 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 144 - protein, 32 aa, synthetic construct
SGGSSGGSSGSETPGTSESATPESSGGSSGGS

SEQ ID NO: 145 - protein, 20 aa, synthetic construct
  VARIANT 4: note = Any naturally occurring amino acid
  VARIANT 8: note = Any naturally occurring amino acid
  VARIANT 11: note = Any naturally occurring amino acid
  VARIANT 15: note = Any naturally occurring amino acid
  VARIANT 19: note = Any naturally occurring amino acid
GLFXALLXLLXSLWXLLLXA

SEQ ID NO: 146-147 - skipped (2 entries)

SEQ ID NO: 148 - protein, 8 aa, synthetic construct
  VARIANT 3: note = Any naturally occurring amino acid
  VARIANT 4: note = Tryptophan, phenylalanine or Tyrosine
  VARIANT 5: note = Any naturally occurring amino acid
  VARIANT 6: note = Any naturally occurring amino acid
KRXXXXAF

SEQ ID NO: 149 - skipped

SEQ ID NO: 150 - protein, 6 aa, synthetic construct
  VARIANT 5: note = Lysine or Arginine
  VARIANT 6: note = Tryptophan, phenylalanine or Tyrosine
LGKRXX

SEQ ID NO: 151-157 - skipped (7 entries)

SEQ ID NO: 158 - protein, 16 aa, synthetic construct
APKKKRKVGIHGVPAA

SEQ ID NO: 159 - DNA, 20 bp, synthetic construct
  source 1..20: mol_type = other DNA
  misc_difference 1..20: note = This sequence may encompass 5-20 nucleotides
aaaaaaaaaaaaaaaaaaaa

SEQ ID NO: 160 - DNA, 20 bp, synthetic construct
  source 1..20: mol_type = other DNA
  misc_difference 1..20: note = This sequence may encompass 5-20 nucleotides
tttttttttttttttttttt

SEQ ID NO: 161 - DNA, 20 bp, synthetic construct
  source 1..20: mol_type = other DNA
  misc_difference 1..20: note = This sequence may encompass 5-20 nucleotides
cccccccccccccccccccc

SEQ ID NO: 162 - DNA, 20 bp, synthetic construct
  source 1..20: mol_type = other DNA
  misc_difference 1..20: note = This sequence may encompass 5-20 nucleotides
gggggggggggggggggggg

SEQ ID NO: 163 - RNA, 41 nt, synthetic construct
  source 1..41: mol_type = other RNA
taatttctactaagtgtagatagctgctcgaattggctttg

SEQ ID NO: 164 - RNA, 40 nt, synthetic construct
  source 1..40: mol_type = other RNA
gaatttctactattgtagataagccaattcgagcagctga

SEQ ID NO: 165 - DNA, 20 bp, synthetic construct
  source 1..20: mol_type = other DNA
tgctaaacttgttgctactg

SEQ ID NO: 166 - DNA, 18 bp, synthetic construct
  source 1..18: mol_type = other DNA
ttcttccgtgaccagtct

SEQ ID NO: 167 - DNA, 42 bp, synthetic construct
  source 1..42: mol_type = other DNA
cgctcgaagagtgaattgaggatgatcttacagtggaggatg

SEQ ID NO: 168 - DNA, 39 bp, synthetic construct
  source 1..39: mol_type = other DNA
ctgcggtgggagtcttatccgtctccctcttctggtgat

SEQ ID NO: 169 - DNA, 19 bp, synthetic construct
  source 1..19: mol_type = other DNA
atccgatggccatcttctt

SEQ ID NO: 170 - DNA, 20 bp, synthetic construct
  source 1..20: mol_type = other DNA
caatttggtcaagagcaccg

SEQ ID NO: 171 - protein, 26 aa, synthetic construct
GSGGSPAGSPTSTEEGTSESATPGSG

(19) United States
(12) Patent Application Publication
(10) Pub. No.: US 2025/0101498 A1
(43) Pub. Date: Mar. 27, 2025

(54) EFFECTOR PROTEINS, COMPOSITIONS, SYSTEMS, DEVICES, KITS AND METHODS OF USE THEREOF

(71) Applicant: Mammoth Biosciences, Inc., Brisbane, CA (US)

(72) Inventors: William Douglass WRIGHT, Fairfield, CA (US); Stepan TYMOSHENKO, Sacramento, CA (US); David PAEZ-ESPINO, Concord, CA (US); Ashley Dorothy AMADO, San Francisco, CA (US); Janice Sha CHEN, San Francisco, CA (US); James Paul BROUGHTON, South San Francisco, CA (US); Xin MIAO, Mountain View, CA (US); Sara ANSALONI, Mountain View, CA (US); Yining ZHANG, Oakland, CA (US); Sierra Hirose LEE, San Francisco, CA (US); Ryan HONG, Hillsborough, CA (US)

(21) Appl. No.: 18/643,955

(22) Filed: Apr. 23, 2024

Related U.S. Application Data
(63) Continuation of application No. PCT/US22/78923, filed on Oct. 28, 2022.
(60) Provisional application No. 63/349,390, filed on Jun. 6, 2022.
(60) Provisional application No. 63/316,822, filed on Mar. 4, 2022.
(60) Provisional application No. 63/282,121, filed on Nov. 22, 2021.
(60) Provisional application No. 63/273,661, filed on Oct. 29, 2021.

(51) Int. Cl. C12Q 1/6818; C12N 9/22; C12N 15/11

(52) U.S. Cl. CPC C12Q 1/6818; C12N 9/22; C12N 15/11; C12N 2310/20

(57) ABSTRACT

Provided herein are compositions, systems, and methods comprising effector proteins and uses thereof. These effector proteins may be characterized as CRISPR-associated (Cas) proteins. Various compositions, systems, and methods of the present disclosure may leverage the activities of these effector proteins for the modification, detection, and engineering of nucleic acids.

### CROSS-REFERENCE

This application is a continuation of International Application No. PCT/US22/78923, filed Oct. 28, 2022, which claims the benefit of U.S. Provisional Application No. 63/273,661, filed Oct. 29, 2021, U.S. Provisional Application No. 63/282,121, filed Nov. 22, 2021, U.S. Provisional Application No. 63/316,822, filed Mar. 4, 2022, and U.S. Provisional Application No. 63/349,390, filed Jun. 6, 2022; the disclosures of which are incorporated herein by reference in their entirety.

### SEQUENCE LISTING

The instant application contains a Sequence Listing, which has been submitted via Patent Center. The Sequence Listing titled 203477-703301-US_SL.xml, which was created on Apr. 17, 2024, and is 276,449 bytes size, is hereby incorporated by reference in its entirety.

### BACKGROUND

Programmable nucleases are proteins that bind and cleave nucleic acids in a sequence-specific manner. A programmable nuclease may bind a target region of a nucleic acid and cleave the nucleic acid within the target region or at a position adjacent to the target region. In some instances, a programmable nuclease is activated when it binds a target region of a nucleic acid to cleave regions of the nucleic acid that are near, but not adjacent to the target region. A programmable nuclease, such as a CRISPR-associated (Cas) protein, may be coupled to a guide nucleic acid that imparts activity or sequence selectivity to the programmable nuclease. In general, guide nucleic acids comprise a CRISPR RNA (crRNA) that is at least partially complementary to a target nucleic acid. In some cases, guide nucleic acids comprise a trans-activating crRNA (tracrRNA) sequence, at least a portion of which interacts with the programmable nuclease. In some cases, a tracrRNA is provided separately from the crRNA and hybridizes to a portion of the crRNA that does not hybridize to the target nucleic acid. In other cases, the tracrRNA and crRNA are linked as a single guide RNA. In other instances, a tracrRNA is not required for Cas protein function.

Programmable nucleases may cleave nucleic acids, including single stranded RNA (ssRNA), double stranded DNA (dsDNA), and single-stranded DNA (ssDNA). Programmable nucleases may provide cis cleavage activity, trans cleavage activity, nickase activity, or a combination thereof. Cis cleavage activity is cleavage of a target nucleic acid that is hybridized to a guide nucleic acid, wherein cleavage occurs within or directly adjacent to the region of the target nucleic acid that is hybridized to guide RNA. Trans cleavage activity is cleavage of ssDNA or ssRNA that is near, but not hybridized to the guide RNA. Trans cleavage activity may be triggered by the hybridization of guide RNA to the target nucleic acid. Nickase activity is the selective cleavage of one strand of a dsDNA molecule.

Programmable nucleases may be modified to have reduced nuclease or nickase activity relative to its unmodified version but retain their sequence selectivity. For instance, amino acid residues of the programmable nuclease that impart catalytic activity to the programmable nuclease may be substituted with an alternative amino acid that does not impart catalytic activity to the programmable nuclease.

While certain programmable nucleases may be used to edit and detect nucleic acid molecules in a sequence specific manner, challenging biological and sample conditions (e.g., high viscosity, metal chelating) may limit their accuracy and effectiveness. There is thus a need for systems and methods that employ programmable nucleases having specificity and efficiency across a wide range of biological and sample conditions.

### SUMMARY

The present disclosure provides compositions, systems, devices, kits, and methods comprising effector proteins and uses thereof. Compositions, systems, devices, kits, and methods disclosed herein leverage nucleic acid modifying activities (e.g., cis cleavage activity and trans cleavage activity) of these effector proteins for the modification, detection, and engineering of target nucleic acids.

Provided herein are systems comprising: a) a polypeptide, or a nucleic acid encoding the polypeptide, wherein the polypeptide comprises an amino acid sequence that is at least 75% identical to any one of the sequences set forth in TABLE 1; and b) an engineered guide nucleic acid, or a nucleic acid that encodes the engineered guide nucleic acid, wherein the engineered guide nucleic acid comprises a first region and a second region, wherein the first region comprises a nucleic acid sequence that is complementary to the target sequence in the target nucleic acid, wherein the first region and the second region are heterologous to each other.

Provided herein are systems comprising: a) a polypeptide, or a nucleic acid encoding the polypeptide, wherein the polypeptide comprises about 100, about 120, about 140, about 160, about 180, about 200, about 220, about 240, about 260, about 280, about 300, about 320, about 340, about 360, about 380, about 400, about 420, about 440, about 460, about 480, about 500, about 520, about 540, about 560, about 580, about 600, about 620, about 640, about 660, about 680, about 700, about 720, about 740, about 760, about 780, about 800, about 820, about 840, about 860, about 880, about 900, about 920, about 940, about 960, about 980, about 1000, about 1020, about 1040, about 1060, about 1080, about 1100, about 1120, about 1140, about 1160, about 1180, about 1200, about 1220, about 1240, about 1260, about 1280, about 1300, about 1320, about 1340, or about 1360 contiguous amino acids of an amino acid sequence selected from any one of the sequences set forth in TABLE 1; and b) an engineered guide nucleic acid, or a nucleic acid that encodes the engineered guide nucleic acid, wherein the engineered guide nucleic acid comprises a first region and a second region, wherein the first region comprises a nucleic acid sequence that is complementary to the target sequence in the target nucleic acid, wherein the first region and the second region are heterologous to each other.

Provided herein are systems comprising: a) a polypeptide, or a nucleic acid encoding the polypeptide, wherein the polypeptide comprises the amino acid sequence located at positions 1-100, 150-250, 101-200, 250-350, 201-300, 350-450, 301-400, 350-450, 401-500, 450-550, 501-600, 550-650, 601-700, 650-750, 701-800, 750-850, 801-900, 850-950, 901-1000, 950-1050, 1001-1100, 1050-1150, 1101-1200, 1150-1250, 1201-1300, or 1250-1350 of a sequence selected from any one of the sequences set forth in TABLE 1; and b) an engineered guide nucleic acid, or a nucleic acid that encodes the engineered guide nucleic acid, wherein the engineered guide nucleic acid comprises a first region and a second region, wherein the first region comprises a nucleic acid sequence that is complementary to the target sequence in the target nucleic acid, wherein the first region and the second region are heterologous to each other.

In some aspects, the polypeptide comprises an amino acid sequence that is at least 80% identical to any one of the sequences set forth in TABLE 1. In some embodiments, the polypeptide comprises an amino acid sequence that is at least 85% identical to any one of the sequences set forth in TABLE 1. In some embodiments, the polypeptide comprises an amino acid sequence that is at least 90% identical to any one of the sequences set forth in TABLE 1. In some embodiments, the polypeptide comprises an amino acid sequence that is at least 95% identical to any one of the sequences set forth in TABLE 1. In some embodiments, the polypeptide comprises an amino acid sequence that is identical to any one of the sequences set forth in TABLE 1. In some embodiments, the sequence of TABLE 1 is selected from the group consisting of SEQ ID NOS: 1-28. In some embodiments, the sequence of TABLE 1 is selected from the group consisting of SEQ ID NOS: 93-142.

In some embodiments, the second region comprises a repeat sequence. In some embodiments, engineered guide nucleic comprises a repeat sequence, wherein the repeat sequence comprises a nucleotide sequence that is at least 75% identical to any one of the sequences set forth in TABLE 4. In some embodiments, the repeat sequence of the engineered guide nucleic acid comprises a nucleotide sequence that is at least 80% identical to any one of the sequences set forth in TABLE 4. In some embodiments, the repeat sequence of the engineered guide nucleic acid comprises a nucleotide sequence that is at least 85% identical to any one of the sequences set forth in TABLE 4. In some embodiments, the repeat sequence of the engineered guide nucleic acid comprises a nucleotide sequence that is at least 90% identical to any one of the sequences set forth in TABLE 4. In some embodiments, the repeat sequence of the engineered guide nucleic acid comprises a nucleotide sequence that is at least 95% identical to any one of the sequences set forth in TABLE 4. In some embodiments, the repeat sequence of the engineered guide nucleic acid comprises a nucleotide sequence that is identical to any one of the sequences set forth in TABLE 4. In some embodiments, the first region of the engineered guide nucleic acid, at least partially, comprises a crRNA. In some embodiments, the crRNA comprises a repeat sequence. In some embodiments, the crRNA comprises a nucleotide sequence that is at least 75%, or at least 80%, or at least 85%, or at least 90% identical to any one of the sequences set forth in TABLE 5. In some embodiments, the engineered guide nucleic acid comprises a spacer sequence. In some embodiments, the first region of the engineered guide nucleic acid comprises the spacer sequence. In some embodiments, the first region comprises at least 10 contiguous nucleotides that are reverse complementary to a eukaryotic sequence. In some embodiments, the engineered guide nucleic acid comprises one or more phosphorothioate (PS) backbone modifications, 2′-fluoro (2′-F) sugar modifications, or 2′-O-Methyl (2′OMe) sugar modifications. In some embodiments, the first region is covalently linked to the second region. In some embodiments, the guide nucleic acid is a single guide nucleic acid, optionally wherein the single guide nucleic acid comprises a nucleotide sequence that is at least 75%, or at least 80%, or at least 85%, or at least 90% identical to any one of the sequences set forth in TABLE 6. In some embodiments, the eukaryotic sequence is a target sequence in a target nucleic acid. In some embodiments, the polypeptide recognizes a PAM. In some embodiments, the target sequence is located adjacent to a protospacer adjacent motif (PAM) sequence in a target nucleic acid. In some embodiments, the PAM comprises any one of the sequences of TABLE 3. In some embodiments, the target nucleic acid is selected from any one of the target nucleic acids set forth in TABLE 7. In some embodiments, the polypeptide is fused to at least one heterologous sequence. In some embodiments, the polypeptide is fused to at least one nuclear localization signal. In some embodiments, the polypeptide is capable of cleaving the target nucleic acid. In some embodiments, the polypeptide is a nuclease that is capable of cleaving at least one strand of a target nucleic acid. In some embodiments, the polypeptide comprises at least one mutation that reduces its nuclease activity, relative to an otherwise comparable polypeptide without the mutation, as measured in a cleavage assay. In some embodiments, the system further comprises a fusion partner fused to the polypeptide or a nucleic acid encodes a fusion partner fused to the polypeptide. In some embodiments, the fusion partner protein is directly fused to the N terminus or C terminus of the polypeptide by an amide bond or by a covalent linker. In some embodiments, the fusion partner protein comprises a polypeptide selected from a deaminase, a transcriptional activator, a transcriptional repressor, or a functional domain thereof. In some embodiments, the system further comprises an additional guide nucleic acid that binds a different loci of the target nucleic acid than the guide nucleic acid. In some embodiments, the system further comprises a donor nucleic acid. In some embodiments, the donor nucleic acid comprises linear double-stranded DNA. In some embodiments, the donor nucleic acid comprises single-stranded DNA. In some embodiments, the donor nucleic acid comprises a nucleotide sequence encoding a functional polypeptide and/or wherein the donor nucleic acid comprises a wildtype sequence. In some embodiments, the donor nucleic acid comprises a protein coding sequence, a gene, a gene fragment, an exon, an intron, an exon fragment, an intron fragment, a gene regulatory region, a gene regulatory region fragment, coding sequences thereof, or combinations thereof. In some embodiments, the polypeptide comprises an activity in a solution comprising salt, wherein the concentration of a salt in the solution is from about 0.001 mM to 200 mM. In some embodiments, the polypeptide comprises an activity in a solution, wherein a temperature of the solution is from about 37° C. to about 65° C. In some embodiments, the activity is modification activity. In some embodiments, the modification activity comprises cleaving at least one strand of a target nucleic acid, deleting or excising one or more nucleotides of the target nucleic acid, inserting one or more nucleotides into the target nucleic acid, substituting one or more nucleotides of the target nucleic acid with one or more alternative nucleotides, or combinations thereof. In some embodiments, the modification activity comprises cleaving at least one strand of a non-target nucleic acid, deleting or excising one or more nucleotides of a non-target nucleic acid, or both. In some embodiments, the system modifies a target nucleic acid. In some embodiments, the system modifies a non-target nucleic acid. In some embodiments, the system modifies a target nucleic acid when a complex comprising the polypeptide and the engineered guide nucleic acid hybridizes to a target sequence in a target nucleic acid. In some embodiments, the engineered guide nucleic acid or a portion thereof hybridizes to a target strand of the target nucleic acid, wherein a PAM is located on a non-target strand of the target nucleic acid, optionally, wherein the PAM is located 5′ of the target sequence on the non-target strand. In some embodiments, the polypeptide comprises an enhanced activity compared to a Cas12 protein. In some embodiments, the system comprises a salt in a solution comprising the polypeptide. In some embodiments, the salt is potassium acetate, sodium chloride, or ammonium sulfate. In some embodiments, the concentration of the salt in the solution is 0.001 mM to 200 mM. In some embodiments, the concentration of the salt in the solution is about 100 mM to about 200 mM. In some embodiments, the system comprises a solution comprising the polypeptide wherein the solution is from about 37° C. to about 65° C. In some embodiments, the solution is from about 40° C. to about 60° C. In some embodiments, the system further comprises one or more of: a detection reagent; and/or an amplification reagent. In some embodiments, the one or more detection reagent is selected from a nucleic acid, optionally wherein the nucleic acid is a detection nucleic acid, a detection moiety, an additional polypeptide, or a combination thereof, optionally wherein the reporter nucleic acid comprises a fluorophore, a quencher, or a combination thereof. In some embodiments, the one or more amplification reagent is selected from the group consisting of a primer, a polymerase, a deoxynucleoside triphosphate (dNTP), a ribonucleoside triphosphate (rNTP), and combinations thereof. In some embodiments, the one or more detection reagent is operably linked to a polypeptide, such that a detection event occurs upon contacting the system with a target nucleic acid.

Also provided herein is a system for detecting a target nucleic acid, comprising any system described herein, and one or more detection reagents, wherein the detection reagent comprises a reporter comprising a reporter nucleic acid and a detection moiety. In some embodiments, cleavage of the reporter generates a detectable product or detectable signal from the detectable moiety. In some embodiments, cleavage of the reporter reduces a detectable signal from the detectable moiety. In some embodiments, cleavage of the reporter is effective to produce a detectable product comprising a detectable moiety. In some embodiments, the detectable moiety comprises a fluorophore, a quencher, a fluorescence resonance energy transfer (FRET) pair, a fluorescent protein, a colorimetric signal, an antigen or a combination thereof. In some embodiments, the reporter comprises a fluorophore which is attached to a quencher by a detector nucleic acid, and wherein, upon cleavage of the detector nucleic acid, the fluorophore generates a signal, wherein the signal is detected as a positive signal, indicating the presence of the target nucleic acid. In some embodiments, the reporter is configured to generate a signal indicative of a presence or absence of the target nucleic acid. In some embodiments, the polypeptide is effective to cleave the reporter in response to formation of a complex comprising the polypeptide, the engineered guide nucleic acid, and the target nucleic acid. In some embodiments, the reporter is configured to release a detection moiety when cleaved by the polypeptide following hybridizing of the guide nucleic acid to the target nucleic acid, and wherein release of the detection moiety is indicative of a presence or absence of the target nucleic acid. In some embodiments, the reporter is operably linked to a polypeptide. In some embodiments, the engineered guide nucleic acid is capable of hybridizing to a target sequence in a target nucleic acid, wherein the target nucleic acid is any one of: a naturally occurring eukaryotic sequence, an engineered eukaryotic sequence, a fragment of a naturally occurring eukaryotic sequence, a fragment of an engineered eukaryotic sequence, and combinations thereof. In some embodiments, the target nucleic acid is isolated from a human cell. In some embodiments, the nucleic acid encoding the polypeptide is a nucleic acid expression vector. In some embodiments, the nucleic acid expression vector is a viral vector. In some embodiments, the nucleic acid expression vector is an adeno associated viral (AAV) vector. In some embodiments, the nucleic acid expression vector encodes at least one guide nucleic acid. In some embodiments, the system is present in a single composition. In some embodiments, the system comprises a device with a chamber or solid support for containing the composition, target nucleic acid, detection reagent or combination thereof. In some embodiments, the system comprises Thermostable Inorganic Pyrophosphatase (TIPP).

Also provided herein are pharmaceutical compositions, comprising a system described herein and a pharmaceutically acceptable excipient.

Also provided herein are methods of detecting a presence of a target nucleic acid in a sample, comprising the steps of: contacting the sample with: any system described herein; and cleaving a reporter with the polypeptide in response to formation of a complex comprising the polypeptide, an engineered guide nucleic acid, and a target sequence in a target nucleic acid, thereby producing a detectable product; and detecting the detectable product, thereby detecting the presence of the target nucleic acid in the sample. In some embodiments, the reporter nucleic acid comprises a fluorophore, a quencher, or a combination thereof, and wherein the detecting comprises detecting a fluorescent signal. In some embodiments, the method comprises reverse transcribing the target nucleic acid, amplifying the target nucleic acid, in vitro transcribing the target nucleic acid, or any combination thereof. In some embodiments, the method comprises reverse transcribing the target nucleic acid and/or amplifying the target nucleic acid before contacting the sample with the composition. In some embodiments, the method comprises reverse transcribing the target nucleic acid and/or amplifying the target nucleic acid after contacting the sample with the composition. In some embodiments, the amplifying comprises isothermal amplification. In some embodiments, the detectable product further comprises a detectable label or a nucleic acid encoding a detectable label selected from a reporter nucleic acid, a detection moiety, an additional polypeptide, or a combination thereof, optionally wherein the reporter nucleic acid comprises a fluorophore, a quencher, or a combination thereof. In some embodiments, the method occurs at a temperature of about 37° C. to about 70° C. In some embodiments, the method occurs at a temperature of about 37° C. to about 65° C. In some embodiments, the method occurs at a temperature of about 37° C. to about 60° C. In some embodiments, the method occurs at a temperature of about 37° C. to about 55° C. In some embodiments, the method occurs at a temperature of about 37° C. to about 50° C. In some embodiments, the method occurs at a temperature of about 37° C. to about 45° C. In some embodiments, the method occurs in a solution, and wherein the solution comprises a salt. In some embodiments, the salt is a potassium salt, ammonium sulfate, or a sodium salt. In some embodiments, the salt is a potassium salt, optionally wherein the potassium salt is potassium acetate. In some embodiments, the salt is a sodium salt, optionally wherein the sodium salt is sodium chloride In some embodiments, the concentration of the salt in the sample is selected from 0.001 mM to 200 mM, 0.01 mM to 200 mM, 0.1 mM to 200 mM, 1 mM to 200 mM, or 10 mM to 200 mM. In some embodiments, the concentration of the salt in the sample is selected from 0.001 mM to 100 mM, 0.01 mM to 100 mM, 0.1 mM to 100 mM, 1 mM to 100 mM, or 10 mM to 100 mM. In some embodiments, the concentration of the target nucleic acid in the sample is selected from 0.001 nM to 100 nM, 0.01 nM to 10 nM, or 0.1 nM to 1 nM. In some embodiments, the target nucleic acid can be detected in less than 20 minutes. In some embodiments, the target nucleic acid can be detected in less than 15 minutes. In some embodiments, the target nucleic acid can be detected in less than 10 minutes. In some embodiments, the target nucleic acid can be detected in less than 5 minutes. In some embodiments, the contacting occurs in vitro. In some embodiments, the contacting occurs ex vivo. In some embodiments, the method comprises contacting the target nucleic acid with the system of any one of claims 1-84, or the pharmaceutical composition of claim 85 thereby producing a modified target nucleic acid. In some embodiments, the method comprises contacting the target nucleic acid with a donor nucleic acid. In some embodiments, the modifying the target nucleic acid comprises insertion or deletion of a sequence of interest, a gene regulatory region, a gene regulatory region fragment, an exon, an intron, an exon fragment, an intron fragment, or any combinations thereof. In some embodiments, the contacting occurs in vivo. In some embodiments, the target sequence is any one of: a naturally occurring eukaryotic sequence, a naturally occurring prokaryotic sequence, a naturally occurring viral sequence, a naturally occurring bacterial sequence, a naturally occurring fungal sequence, an engineered eukaryotic sequence, an engineered prokaryotic sequence, an engineered viral sequence, an engineered bacterial sequence, an engineered fungal sequence, a fragment of a naturally occurring sequence, a fragment of an engineered sequence, and combinations thereof. In some embodiments, the target nucleic acid is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell. In some embodiments, the target nucleic acid comprises RNA. In some embodiments, the target nucleic acid comprises DNA. In some embodiments, the target nucleic acid is from a pathogen. In some embodiments, the pathogen is a virus. In some embodiments, the target nucleic acid comprises a mutation associated with a disease or disorder. In some embodiments, the target nucleic acid comprises one or more mutations. In some embodiments, the one or more mutations comprise a point mutation, a single nucleotide polymorphism (SNP), a chromosomal mutation, a copy number mutation, or any combination thereof. In some embodiments, the disease or disorder is any one of the diseases or disorders recited in TABLE 7. In some embodiments, the modified target nucleic acid no longer comprises a mutation associated with a disease or disorder as compared to an unmodified target nucleic acid. In some embodiments, the modified target nucleic acid no longer comprises sequence markers associated with a disease or disorder as compared to an unmodified target nucleic acid. In some embodiments, the modified target nucleic acid comprises an engineered nucleic acid sequence that expresses a polypeptide having new activity as compared to an unmodified target nucleic acid, or alters expression of an endogenous polypeptide as compared to an unmodified target nucleic acid. In some embodiments, the contacting occurs in vitro.

Also provided herein are methods of treating a disease or disorder associated with a mutation or aberrant expression of a gene in a subject in need thereof, the method comprising administering to the subject a pharmaceutical composition described herein. In some embodiments, contacting the target nucleic acid comprises contacting a cell, wherein the target nucleic acid is located in the cell.

Also provided herein is a cell comprising a target nucleic acid, wherein the cell is contacted by: a system described herein; a pharmaceutical composition described herein; or a method described herein. In some embodiments, upon contacting the cell, the target nucleic acid is thereby modified. In some embodiments, the cell is a eukaryotic cell. In some embodiments, the cell is a mammalian cell. In some embodiments, the cell is a prokaryotic cell. In some embodiments, the cell is a plant cell. In some embodiments, the cell is an animal cell.

Also provided herein is a population of cells comprising at least one cell described herein.

Also provided herein are methods of producing a protein, the method comprising, contacting a cell as described herein, thereby modifying a target nucleic acid; and producing a protein from the cell that is encoded, transcriptionally affected, or translationally affected by the modified target nucleic acid.

Also provided herein are methods of treating a disease comprising administering to a subject in need thereof: a system described herein; a pharmaceutical composition described herein; or cell described herein; or a population of cells described herein.

Also provided herein are systems comprising: (a) a polypeptide, or a nucleic acid encoding the polypeptide, and an engineered guide nucleic acid, or a nucleic acid that encodes the engineered guide nucleic acid; (b) a polypeptide, or a nucleic acid encoding the polypeptide, and an engineered guide nucleic acid comprising a single guide nucleic acid; (c) a polypeptide, or a nucleic acid encoding the polypeptide, an engineered guide nucleic acid, and a donor nucleic acid; (d) a polypeptide, or a nucleic acid encoding the polypeptide, an engineered guide nucleic acid comprising a single guide nucleic acid, and a donor nucleic acid; (e) an mRNA encoding a polypeptide, and an engineered guide nucleic acid; (f) an mRNA encoding a polypeptide, an engineered guide RNA, and a donor nucleic acid; (g) one or more recombinant expression vectors comprising: i) a nucleic acid encoding a polypeptide; and ii) an engineered guide nucleic acid; (h) one or more recombinant expression vectors comprising: i) a nucleic acid encoding a polypeptide; ii) an engineered guide nucleic acid; and iii) and a donor nucleic acid; (i) one or more recombinant expression vectors comprising: i) a nucleic acid encoding a polypeptide; and ii) an engineered guide nucleic acid comprising a single guide nucleic acid; or (j) one or more recombinant expression vectors comprising: i) a nucleic acid encoding a polypeptide; ii) an engineered guide nucleic acid comprising a single guide nucleic acid; and iii) and a donor nucleic acid; wherein the polypeptide comprises an amino acid sequence that is at least 85% identical to any one of the sequences set forth in TABLE 1.

Also provided herein are kits comprising: (a) a polypeptide, or a nucleic acid encoding the polypeptide, and an engineered guide nucleic acid, or a nucleic acid that encodes the engineered guide nucleic acid; (b) a polypeptide, or a nucleic acid encoding the polypeptide, and an engineered guide nucleic acid comprising a single guide nucleic acid; (c) a polypeptide, or a nucleic acid encoding the polypeptide, an engineered guide nucleic acid, and a donor nucleic acid; (d) a polypeptide, or a nucleic acid encoding the polypeptide, an engineered guide nucleic acid comprising a single guide nucleic acid, and a donor nucleic acid; (e) an mRNA encoding a polypeptide, and an engineered guide nucleic acid; (f) an mRNA encoding a polypeptide, an engineered guide RNA, and a donor nucleic acid; (g) one or more recombinant expression vectors comprising: i) a nucleic acid encoding a polypeptide; and ii) an engineered guide nucleic acid; (h) one or more recombinant expression vectors comprising: i) a nucleic acid encoding a polypeptide; ii) an engineered guide nucleic acid; and iii) and a donor nucleic acid; (i) one or more recombinant expression vectors comprising: i) a nucleic acid encoding a polypeptide; and ii) an engineered guide nucleic acid comprising a single guide nucleic acid; or (j) one or more recombinant expression vectors comprising: i) a nucleic acid encoding a polypeptide; ii) an engineered guide nucleic acid comprising a single guide nucleic acid; and iii) and a donor nucleic acid; wherein the polypeptide comprises an amino acid sequence that is at least 85% identical to any one of the sequences set forth in TABLE 1. In some embodiments, the components of the kit are in same container. In some embodiments, the components of the kit are in separate containers.

Also provided herein are containers comprising: (a) a polypeptide, or a nucleic acid encoding the polypeptide, and an engineered guide nucleic acid, or a nucleic acid that encodes the engineered guide nucleic acid; (b) a polypeptide, or a nucleic acid encoding the polypeptide, and an engineered guide nucleic acid comprising a single guide nucleic acid; (c) a polypeptide, or a nucleic acid encoding the polypeptide, an engineered guide nucleic acid, and a donor nucleic acid; (d) a polypeptide, or a nucleic acid encoding the polypeptide, an engineered guide nucleic acid comprising a single guide nucleic acid, and a donor nucleic acid; (e) an mRNA encoding a polypeptide, and an engineered guide nucleic acid; (f) an mRNA encoding a polypeptide, an engineered guide RNA, and a donor nucleic acid; (g) one or more recombinant expression vectors comprising: i) a nucleic acid encoding a polypeptide; and ii) an engineered guide nucleic acid; (h) one or more recombinant expression vectors comprising: i) a nucleic acid encoding a polypeptide; ii) an engineered guide nucleic acid; and iii) and a donor nucleic acid; (i) one or more recombinant expression vectors comprising: i) a nucleic acid encoding a polypeptide; and ii) an engineered guide nucleic acid comprising a single guide nucleic acid; or (j) one or more recombinant expression vectors comprising: i) a nucleic acid encoding a polypeptide; ii) an engineered guide nucleic acid comprising a single guide nucleic acid; and iii) and a donor nucleic acid; wherein the polypeptide comprises an amino acid sequence that is at least 85% identical to any one of the sequences set forth in TABLE 1. In some embodiments, the container is selected from a syringe, well, bottle, vial, and test tubes, chamber, and channel.

Also provided herein are devices comprising: (a) a polypeptide, or a nucleic acid encoding the polypeptide, and an engineered guide nucleic acid, or a nucleic acid that encodes the engineered guide nucleic acid; (b) a polypeptide, or a nucleic acid encoding the polypeptide, and an engineered guide nucleic acid comprising a single guide nucleic acid; (c) a polypeptide, or a nucleic acid encoding the polypeptide, an engineered guide nucleic acid, and a donor nucleic acid; (d) a polypeptide, or a nucleic acid encoding the polypeptide, an engineered guide nucleic acid comprising a single guide nucleic acid, and a donor nucleic acid; (e) an mRNA encoding a polypeptide, and an engineered guide nucleic acid; (f) an mRNA encoding a polypeptide, an engineered guide RNA, and a donor nucleic acid; (g) one or more recombinant expression vectors comprising: i) a nucleic acid encoding a polypeptide; and ii) an engineered guide nucleic acid; (h) one or more recombinant expression vectors comprising: i) a nucleic acid encoding a polypeptide; ii) an engineered guide nucleic acid; and iii) and a donor nucleic acid; (i) one or more recombinant expression vectors comprising: i) a nucleic acid encoding a polypeptide; and ii) an engineered guide nucleic acid comprising a single guide nucleic acid; or (j) one or more recombinant expression vectors comprising: i) a nucleic acid encoding a polypeptide; ii) an engineered guide nucleic acid comprising a single guide nucleic acid; and iii) and a donor nucleic acid; wherein the polypeptide comprises an amino acid sequence that is at least 85% identical to any one of the sequences set forth in TABLE 1. In some embodiments, the device is used in diagnosis of a disease or disorder associated with a nucleic acid sequence modification in a disease or disorder associated gene selected from a viral genome, a prokaryotic genome, or a eukaryotic genome. In some embodiments, the device is used in diagnosis of a disease or disorder associated with a non-wild type gene, a gene comprising a non-wild type reading frame; a gene comprising one or more mutations, or abnormal processing upon transcription of a gene.

Also provided herein are microfluidic devices comprising: a) a sample interface configured to receive a sample comprising nucleic acids; b) a chamber fluidically connected to the sample interface; wherein the chamber comprises a polypeptide and an engineered guide nucleic acid, wherein the polypeptide comprises an amino acid sequence that is at least 85% identical to any one of the sequences set forth in TABLE 1. In some embodiments, the chamber further comprises a reporter comprising a nucleic acid and a detection moiety. In some embodiments, the polypeptide is effective to form an activated complex with the engineered guide nucleic acid upon hybridization of the engineered guide nucleic acid to a target sequence of a target nucleic acid and wherein the nucleic acid of the reporter is a cleavage substrate of the activated complex. In some embodiments, the reporter is immobilized to a surface within the chamber. In some embodiments, the nucleic acid of the reporter comprises at least one ribonucleotide, at least one deoxyribonucleotide, or at least one ribonucleotide and at least one deoxyribonucleotide. In some embodiments, microfluidic devices further comprise a valve disposed between the sample interface and the chamber, optionally wherein the valve is configured to selectively resist flow, or permit flow. In some embodiments, the chamber further comprises one or more reagents for amplification, one or more cell lysis reagents, one or more nucleic acid purification reagents. In some embodiments, the chamber further comprises a polymerase. In some embodiments, the chamber is a first chamber and the microfluidic device further comprising a second chamber comprising one or more reagents for amplification, one or more cell lysis reagents, one or more nucleic acid purification reagents. In some embodiments, microfluidic devices further comprise a channel comprising one or more reagents for amplification, one or more cell lysis reagents, one or more nucleic acid purification reagents. In some embodiments, the second chamber or channel is disposed between the sample interface and the first chamber, wherein the second chamber or channel is disposed downstream of the sample interface and the first chamber, wherein the second chamber or channel is disposed upstream of the sample interface and the first chamber. In some embodiments, microfluidic devices further comprise a detection region fluidically connected to the first chamber. In some embodiments, the detection region comprises an array, one or more lateral flow strips, a detection tray, a detection region comprising a capture antibody, or combinations thereof.

Also provided herein is the use of the components of any of the systems described herein, any of the kits described herein, any of the containers described herein, any of the devices described herein and/or any of the microfluidic devices described herein, wherein the components of the system, kit, container, device, or microfluidic device are used in the diagnosis of a disease or disorder.

Also provided herein is the use of the components of any of the systems described herein, any of the kits described herein, any of the containers described herein, any of the devices described herein and/or any of the microfluidic devices described herein, wherein the components of the system, kit, container, device, or microfluidic device are used in the diagnosis of a disease or disorder, and wherein the disease or disorder is associated with a nucleic acid sequence modification in a disease or disorder associated gene selected from a viral genome, a prokaryotic genome, or an eukaryotic genome.

Also provided herein is the use of the components of any of the systems described herein, any of the kits described herein, any of the containers described herein, any of the devices described herein and/or any of the microfluidic devices described herein, wherein the components of the system, kit, container, device, or microfluidic device are used in the diagnosis of a disease or disorder, and wherein the disease or disorder is associated with a non-wild type gene, a gene comprising a non-wild type reading frame; a gene comprising one or more mutations, or abnormal processing upon transcription of a gene.

Also provided herein are methods for diagnosis comprising the use of any of the systems described herein, any of the pharmaceutical compositions described herein, any of the kits described herein, any of the containers described herein, any of the devices described herein and/or any of the microfluidic devices described herein, wherein the components of the system, kit, container, device, or microfluidic device further comprises a detectable label or a nucleic acid encoding a detectable label capable of hybridizing to a target nucleic acid. In some embodiments, the hybridizing to a target nucleic acid results in modification of a detectable label and wherein the detectable label emits a detectable signal upon modification. In some embodiments, the target nucleic acid is in one or more of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell.

Also provided herein are compositions comprising: a polypeptide, or a nucleic acid encoding the polypeptide, and an engineered guide nucleic acid, or a nucleic acid that encodes the engineered guide nucleic acid; a polypeptide, or a nucleic acid encoding the polypeptide, and an engineered guide nucleic acid comprising a single guide nucleic acid; a polypeptide, or a nucleic acid encoding the polypeptide, an engineered guide nucleic acid, and a donor nucleic acid; a polypeptide, or a nucleic acid encoding the polypeptide, an engineered guide nucleic acid comprising a single guide nucleic acid, and a donor nucleic acid; an mRNA encoding a polypeptide, and an engineered guide nucleic acid; an mRNA encoding a polypeptide, an engineered guide RNA, and a donor nucleic acid; one or more recombinant expression vectors comprising: i) a nucleic acid encoding a polypeptide; and ii) an engineered guide nucleic acid; one or more recombinant expression vectors comprising: i) a nucleic acid encoding a polypeptide; ii) an engineered guide nucleic acid; and iii) and a donor nucleic acid; one or more recombinant expression vectors comprising: i) a nucleic acid encoding a polypeptide; and ii) an engineered guide nucleic acid comprising a single guide nucleic acid; or one or more recombinant expression vectors comprising: i) a nucleic acid encoding a polypeptide; ii) an engineered guide nucleic acid comprising a single guide nucleic acid; and iii) and a donor nucleic acid; wherein the polypeptide comprises an amino acid sequence that is at least 85% identical to any one of the sequences set forth in TABLE 1.

Also provided herein is the use of any of the systems described herein, any of the pharmaceutical compositions described herein, any of the kits described herein, any of the containers described herein, any of the devices described herein and/or any of the microfluidic devices described herein, wherein the polypeptide comprises an amino acid sequence that is 100% identical to SEQ ID NO: 4. In some embodiments, the target nucleic acid is in a solution, wherein the solution has a sodium chloride concentration of 100 mM to 200 mM, a temperature of about 40° C. to about 60° C., and a 1 nM concentration of the target nucleic acid.

Also provided herein is the use of any of the systems described herein, any of the pharmaceutical compositions described herein, any of the kits described herein, any of the containers described herein, any of the devices described herein and/or any of the microfluidic devices described herein, wherein the polypeptide comprises an amino acid sequence that is 100% identical to SEQ ID NO: 3. In some embodiments, the target nucleic acid is in a solution, wherein the solution has a sodium chloride concentration of 100 mM to 200 mM and a temperature of about 50° C. to about 65° C., and a 0.1 nM concentration of the target nucleic.

Also provided herein is the use of any of the systems described herein, any of the pharmaceutical compositions described herein, any of the kits described herein, any of the containers described herein, any of the devices described herein and/or any of the microfluidic devices described herein, wherein the polypeptide comprises an amino acid sequence that is 100% identical to SEQ ID NO: 6. In some embodiments, the target nucleic acid is in a solution, wherein the solution has a sodium chloride concentration of 100 mM to 200 mM and a temperature of about 55° C. to about 60° C., and a 0.1 nM concentration of the target nucleic.

Also provided herein is the use of any of the systems described herein, any of the pharmaceutical compositions described herein, any of the kits described herein, any of the containers described herein, any of the devices described herein and/or any of the microfluidic devices described herein, wherein the polypeptide comprises an amino acid sequence that is 100% identical to SEQ ID NO: 7. In some embodiments, the target nucleic acid is in a solution, wherein the solution has a sodium chloride concentration of 100 mM to 200 mM and a temperature of about 60° C. to about 70° C., and a 0.1 nM concentration of the target nucleic.

Also provided herein is the use of any of the systems described herein, any of the pharmaceutical compositions described herein, any of the kits described herein, any of the containers described herein, any of the devices described herein and/or any of the microfluidic devices described herein, wherein the polypeptide comprises an amino acid sequence that is 100% identical to SEQ ID NO: 5. In some embodiments, the target nucleic acid is in a solution, wherein the solution has an ammonium sulfate concentration of 100 mM to 200 mM and a temperature of about 50° C. to about 65° C., and 2 μL of the target nucleic.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows exemplary effector protein trans cleavage activity at 37° C.

FIG. 2 shows exemplary maximum rates of trans cleavage activity of effector proteins at 37° C.

FIG. 3A shows exemplary normalized rates of trans cleavage activity of effector proteins at temperatures ranging from 40° C. to 90° C.

FIG. 3B shows exemplary non-normalized rates of trans cleavage activity of effector proteins at temperatures ranging from 40° C. to 90° C.

FIG. 4A shows exemplary normalized rates of trans cleavage activity of effector proteins varying concentrations of potassium acetate.

FIG. 4B shows exemplary non-normalized rates of trans cleavage activity of effector proteins in varying concentrations of potassium acetate.

FIG. 5A shows exemplary maximum rate of trans cleavage activity of effector proteins in the presence of 100 mM sodium chloride at 40° C.

FIG. 5B shows exemplary maximum rate of trans cleavage activity of effector proteins in the presence of 100 mM sodium chloride at 45° C.

FIG. 5C shows exemplary maximum rate of trans cleavage activity of effector proteins in the presence of 100 mM sodium chloride at 50° C.

FIG. 5D shows exemplary maximum rate of trans cleavage activity of effector proteins in the presence of 100 mM sodium chloride at 55° C.

FIG. 5E shows exemplary maximum rate of trans cleavage activity of effector proteins in the presence of 100 mM sodium chloride at 60° C.

FIG. 6A shows exemplary PAM sequence preferences of effector proteins under high stringency conditions (1% cutoff).

FIG. 6B shows exemplary PAM sequence preferences of effector proteins under low stringency conditions (10% cutoff).

FIG. 7 shows exemplary spacer length preferences of effector proteins at 50° C.

FIG. 8A shows exemplary repeat preferences of effector proteins at 50° C. after 10 minutes.

FIG. 8B shows exemplary repeats and effector protein systems detection of the target at different time points at 50° C. All systems can detect targets after 10 minutes.

FIG. 9A shows exemplary effector protein limit of detection of various concentration of targets at 50° C.

FIG. 9B shows exemplary effector protein detection of target over time at 50° C. All effector proteins can detect target as low at 0.01 nm in less than 10 minutes.

FIG. 10A shows exemplary reporter preferences of effector proteins at 50° C.

FIG. 10B shows effector protein cleavage of exemplary reporters over time at 50° C.

FIG. 11A shows exemplary results of three effector protein-detection assays where 1 μL (top) or 2 μL (bottom) of amplification product from an RT-LAMP amplification assay, containing 0 copies (0 cp) or 100 copies (100 cp) of the Influenza B (IVB) target, was titrated into the detection reaction.

FIG. 11B shows exemplary results of three effector protein-detection assays where 3 μL (top) or 4 μL (bottom) of amplification product from an RT-LAMP amplification assay, containing 0 copies (0 cp) or 100 copies (100 cp) of the IVB target, was titrated into the detection reaction.

FIG. 11C shows exemplary results of three effector protein-detection assays where 5 μL (top) or 6 μL (bottom) of amplification product from an RT-LAMP amplification assay, containing 0 copies (0 cp) or 100 copies (100 cp) of the IVB target, was titrated into the detection reaction.

FIG. 11D shows exemplary results of three effector protein-detection assays where 7 μL (top) or 8 μL (bottom) of amplification product from an RT-LAMP amplification assay, containing 0 copies (0 cp) or 100 copies (100 cp) of the IVB target, was titrated into the detection reaction.

FIG. 12A shows exemplary results from the generation of the RT-LAMP amplification product of various targets in RT-LAMP-DETECTR one-pot assays. Amplification was monitored via generation of a SYTO9 fluorescent signal.

FIG. 12B shows exemplary results from the concurrent detection of the amplified target nucleic acids in the RT-LAMP-DETECTR one-pot assays of FIG. 12A. Effector protein-based detection was monitored via generation of an Alexa594 fluorescent signal.

FIG. 13A shows exemplary results from the generation of the RT-LAMP amplification product from varying starting concentrations (0 copies (0 cp), 10 copies (10 cp), 25 copies (25 cp), 50 copies (50 cps) or 100 copies (100 cp)) of a RSVB target in RT-LAMP-DETECTR one-pot assays. Amplification was monitored via generation of a SYTO9 fluorescent signal.

FIG. 13B shows exemplary results from the concurrent detection of the target nucleic acid amplification product generated from varying starting concentrations (0 copies (0 cp), 10 copies (10 cp), 25 copies (25 cp), 50 copies (50 cps) or 100 copies (100 cp)) of a RSVB target in the RT-LAMP-DETECTR one-pot assays of FIG. 13A. Effector protein-based detection was monitored via generation of an Alexa594 fluorescent signal.

FIG. 14A shows exemplary results from the generation of the RT-LAMP amplification product from varying starting concentrations (0 copies (0 cp), 10 copies (10 cp), 25 copies (25 cp), 50 copies (50 cps) or 100 copies (100 cp)) of a RNaseP target in RT-LAMP-DETECTR one-pot assays. Amplification was monitored via generation of a SYTO9 fluorescent signal.

FIG. 14B shows exemplary results from the concurrent detection of the target nucleic acid amplification product generated from varying starting concentrations (0 copies (0 cp), 10 copies (10 cp), 25 copies (25 cp), 50 copies (50 cps) or 100 copies (100 cp)) of a RNaseP target in the RT-LAMP-DETECTR one-pot assays of FIG. 14A. Effector protein-based detection was monitored via generation of an Alexa594 fluorescent signal.

FIG. 15A shows exemplary results from the generation of the RT-LAMP amplification product from varying starting concentrations of a RNaseP target (0 copies (0 cp) or 300 copies (300 cp)) and/or a RSVB target (0 copies (0 cp), 75 copies (75 cp), 150 copies (150 cp), or 300 copies (300 cp)) from a nasal fluid sample in RT-LAMP-DETECTR one-pot assays. Amplification was monitored via generation of a SYTO9 fluorescent signal.

FIG. 15B shows exemplary results from the concurrent detection of the amplification product generated from varying starting concentrations of a RNaseP target (0 copies (0 cp) or 300 copies (300 cp)) and a RSVB target (0 copies (0 cp), 75 copies (75 cp), 150 copies (150 cp), or 300 copies (300 cp)) from a nasal fluid sample in the RT-LAMP-DETECTR one-pot assays of FIG. 15A. Effector protein-based detection was monitored via generation of an Alexa594 fluorescent signal.

### DETAILED DESCRIPTION

It is to be understood that both the foregoing general description and the following detailed description are exemplary, and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

### Definitions

Unless otherwise indicated, all technical terms used herein have the same meaning as commonly understood by one of ordinary skill in the art to which this invention belongs. Unless otherwise indicated or obvious from context, the following terms have the following meanings:

As used in this specification and the appended claims, the singular forms “a,” “an,” and “the” include plural references unless the context clearly dictates otherwise.

Any reference to “or” herein is intended to encompass “and/or” unless otherwise stated. As used herein, the term “and/or” includes any and all combinations of one or more of the associated listed items.

Use of the term “including” as well as other forms, such as “includes” and “included,” is not limiting.

As used herein, the term “comprise” and its grammatical equivalents specifies the presence of stated features, integers, steps, operations, elements, and/or components, but does not preclude the presence or addition of one or more other features, integers, steps, operations, elements, components, and/or groups thereof.

As used herein, the term “about” in reference to a number or range of numbers is understood to mean the stated number and numbers +/−10% thereof, or 10% below the lower listed limit and 10% above the higher listed limit for the values listed for a range.

The terms “percent identity,” “% identity,” and % “identical,” as used herein, refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 March; 4 (1): 11-7), FASTA (Pearson and Lipman, Proc Natl Acad Sci USA. 1988 April; 85 (8): 2444-8; Pearson, Methods Enzymol. 1990; 183:63-98) and gapped BLAST (Altschul et al., Nucleic Acids Res. 1997 Sep. 1; 25 (17): 3389-40), BLASTP, BLASTN, or GCG (Devereux et al., Nucleic Acids Res. 1984 Jan. 11; 12 (1 Pt 1): 387-95).

The term, “% similarity,” as used herein, in the context of an amino acid sequence, refers to a value that is calculated by dividing a similarity score by the length of the alignment. The similarity of two amino acid sequences can be calculated by using a BLOSUM62 similarity matrix (Henikoff and Henikoff, Proc. Natl. Acad. Sci. USA., 89:10915-10919 (1992)) that is transformed so that any value ≥1 is replaced with +1 and any value ≤0 is replaced with 0. For example, an Ile (I) to Leu (L) substitution is scored at +2.0 by the BLOSUM62 similarity matrix, which in the transformed matrix is scored at +1. This transformation allows the calculation of percent similarity, rather than a similarity score. Alternately, when comparing two full protein sequences, the proteins can be aligned using pairwise MUSCLE alignment. Then, the % similarity can be scored at each residue and divided by the length of the alignment. For determining % similarity over a protein domain or motif, a multilevel consensus sequence (or PROSITE motif sequence) can be used to identify how strongly each domain or motif is conserved. In calculating the similarity of a domain or motif, the second and third levels of the multilevel sequence are treated as equivalent to the top level. Additionally, if a substitution could be treated as conservative with any of the amino acids in that position of the multilevel consensus sequence, +1 point is assigned. For example, given the multilevel consensus sequence: RLG and YCK, the test sequence QIQ would receive three points. This is because in the transformed BLOSUM62 matrix, each combination is scored as: Q-R: +1; Q-Y: +0; I-L: +1; I-C: +0; Q-G: +0; Q-K: +1. For each position, the highest score is used when calculating similarity. The % similarity can also be calculated using commercially available programs, such as the Geneious Prime software given the parameters matrix=BLOSUM62 and threshold ≥1.

The term “amplification” and “amplifying,” as used herein, refers to a process by which a nucleic acid molecule is enzymatically copied to generate a plurality of nucleic acid molecules containing the same sequence as the original nucleic acid molecule or a distinguishable portion thereof.

The term “cancer,” as used herein, can refer to a disease state characterized by the presence in a subject of cells demonstrating abnormal uncontrolled replication. The term cancer may be used interchangeably with the terms “carcino-,” “onco-,” and “tumor.”

The term “capture molecule”, “capture antibody” and the like, as used herein, generally refers to a molecule that selectively binds to a target nucleic acid and only nonspecifically binds to other nucleic acids that can be washed away.

The term, “chamber,” and “channel,” when used interchangeably herein, in reference to a device, such as a microfluidic device, refers to a compartment, which is at least partially enclosed, in the device, where an activity, such as a reaction, can occur. A chamber or channel is generally connected or communicating with another component of the device. A chamber or channel may contain or have the ability to contain matter, such as reagents. Alternatively or in addition, a chamber or channel can also direct or vent air or gases. By way of non-limiting example, the chamber or channels may comprise one or more hydrogels, a well, a flow strip, a heating element, or combinations thereof. Also, by way of non-limiting example, the chamber or channels may be in fluid communication, optical communication, or thermal communication. As another non-limiting example, the chamber or channels may be arranged in a sequence, in parallel, or both.

As used herein, the term “cis cleavage” when used in reference to cleavage (hydrolysis of a phosphodiester bond) of a target nucleic acid by an effector protein complexed with a guide nucleic acid refers to cleavage of a target nucleic acid that is hybridized to a guide nucleic acid, wherein cleavage occurs within or directly adjacent to the region of the target nucleic acid that is hybridized to the guide nucleic acid.

The terms “cleave,” “cleaving,” and “cleavage,” as used herein, with reference to a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The term “cleavage assay,” as used herein, refers to an assay designed to visualize, quantitate, or identify cleavage of a nucleic acid. In some instances, the cleavage activity may be cis cleavage activity. In some instances, the cleavage activity may be trans-cleavage activity.

The term “complementary,” as used herein with reference to a nucleic acid, refers to the characteristic of a polynucleotide having nucleotides that base pair with their Watson-Crick counterparts (C with G; or A with T) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The term “CRISPR-RNA” or “crRNA” refers to a nucleic acid comprising a sequence, often referred to as a “spacer sequence,” with sufficient complementarity to a target nucleic acid sequence to direct sequence-specific binding of a complex of an effector protein and a guide nucleic acid to the target nucleic acid sequence. In some instances, crRNAs contain a sequence that mediates target recognition and a sequence that duplexes with a tracrRNA. In some instances, the crRNA and tracrRNA duplex are present as parts of a single larger guide RNA molecule. In some instances, the crRNA comprises a sequence that is recognized by and bound by an effector protein. In some instances, the crRNA comprises a repeat sequence.

The term “detectable signal,” as used herein, refers to a signal that can be discovered, identified, perceived or noticed using optical, fluorescent, chemiluminescent, electrochemical, or other detection methods known in the art.

The term, “detectable product” refers to a molecule produced after the cleavage of a reporter that is capable of being discovered, identified, perceived or noticed. A detectable product can comprise a detectable label and/or moiety that emits a detectable signal. A detectable product may include other components that are not capable of being readily discovered, identified, perceived or noticed at the same time as the detectable signal. For example, a detectable product may comprise remnants of the reporter. Accordingly, in some instances, the detectable product comprises RNA and/or DNA.

The term, “detection event” refers to the activity (e.g., cleavage) that occurs between a target (e.g., a target nucleic acid) and one or more components for detection (e.g., a reporter, detectable moiety, and/or detectable label), which causes the generation of a signal (e.g., a detectable signal or detectable product) that indicates that the activity has occurred.

The term, “detection region,” as used herein, refers to an array, one or more lateral flow strips, a detection tray, a capture antibody, or combinations thereof.

The term “DETECTR,” or “DNA endonuclease targeted CRISPR trans reporter (DETECTR)” as used herein, refers to an assay that determines the presence of a target nucleic acid sequence is a sample by detecting effector protein-based reporter cleavage (directly or indirectly). Such assays can leverage the trans cleavage properties of effector protein enzymes (e.g., CRISPR-Cas enzymes).

The terms “determining,” “measuring,” “evaluating,” “assessing,” “assaying,” and “analyzing” are used interchangeably herein, unless otherwise indicated, to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “donor nucleic acid” refers to a sequence of nucleotides that will be or has been introduced or incorporated into a target nucleic acid or cell. For example, when used in reference to a viral vector, the term donor nucleic acid refers to a sequence of nucleotides that will be or has been introduced into a cell following transfection of the viral vector. The donor nucleic acid can be introduced into the cell by any mechanism of the transfecting viral vector, including, but not limited to, integration into the genome of the cell or introduction of an episomal plasmid or viral genome. As another example, when used in reference to the activity of an effector protein, the term donor nucleic acid refers to a sequence of nucleotides that will be or has been inserted at the site of cleavage by the effector protein (cleaving (hydrolysis of a phosphodiester bond) of a nucleic acid molecule resulting in a nick or double strand break-endonuclease activity). As yet another example, when used in reference to homologous recombination, the term donor nucleic acid refers to a sequence of DNA that serves as a template in the process of homologous recombination, which can carry the modification that is to be or has been introduced into the target nucleic acid. By using this donor nucleic acid as a template, the genetic information, including the modification, is copied into the target nucleic acid by way of homologous recombination.

As used herein, the term “donor nucleotide” refers to a single nucleotide that will be or has been introduced or incorporated into a target nucleic acid or cell. The donor nucleotide can be part of a larger sequence of nucleotides, such as a doner nucleic acid, or is a single nucleotide. Like a donor nucleic acid, the donor nucleotide, when used in reference to the activity of an effector protein, the term donor nucleotide refers to a nucleotide that will be or has been inserted at the site of cleavage by the effector protein (e.g., cleaving (hydrolysis of a phosphodiester bond) of a nucleic acid molecule resulting in a nick or double strand break-endonuclease activity).

The term “dual nucleic acid system” as used herein refers to a system that uses a transactivated or transactivating tracrRNA-crRNA duplex complexed with one or more polypeptides described herein, wherein the complex is capable of interacting with a target nucleic acid in a sequence selective manner.

The term, “edited target nucleic acid,” as used herein, refers to a target nucleic acid, wherein the target nucleic acid has undergone an editing, for example, after contact with an effector protein. In some instances, the editing is an alteration in the sequence of the target nucleic acid. In some instances, the edited target nucleic acid comprises an insertion, deletion, or substitution of one or more nucleotides compared to the unedited target nucleic acid.

The term “effector protein,” as used herein, refers to a protein, polypeptide, or peptide that is capable of binding to a guide nucleic acid and/or modifying a nucleic acid molecule (e.g., cleaving (hydrolysis of a phosphodiester bond) of a nucleic acid molecule resulting in a nick or double strand break-nuclease activity; or breaking of hydrogen bonds between annealed nucleotide bases of a nucleic acid molecule-helicase activity). A nucleic acid molecule that an effector protein can modify includes, for example, a target nucleic acid molecule or a pre-crRNA. An effector protein can modify a nucleic acid molecule by cis cleavage or trans cleavage. An effector protein can also be capable of binding to a target nucleic acid molecule in the presence of a guide nucleic acid, wherein the guide nucleic acid includes a sequence that is complementary with an equal length portion of the target nucleic acid. The ability of an effector protein to modify a nucleic acid molecule can be dependent upon the effector protein being bound to a guide nucleic acid and the guide nucleic acid being hybridized to a target nucleic acid molecule. The modification of the target nucleic acid molecule generated by an effector protein can, as a non-limiting example, result in modulation of the expression of the nucleic acid molecule (e.g., increasing or decreasing expression of the nucleic acid molecule) or modulation of the activity of a translation product of the target nucleic acid (e.g., inactivation of a protein binding to an RNA molecule or hybridization). An effector protein can be a CRISPR-associated (“Cas”) protein. An effector protein can function as a single protein, including a single protein that is capable of binding to a guide nucleic acid and modifying a nucleic acid molecule (e.g., a Type II, Type V, or Type VI effector complex). Alternatively, an effector protein can function as part of a multiprotein complex, including, for example, a complex having two or more effector proteins (e.g., a Type I, Type III, Type IV effector complex). An effector protein when functioning in a multiprotein complex can have only one functional activity (e.g., binding to a guide nucleic acid), while other effector proteins present in the multiprotein complex are capable of the other functional activity (e.g., modifying a nucleic acid molecule). An effector protein can be a modified effector protein having reduced (e.g., a catalytically defective effector protein) or no nuclease activity (e.g., a catalytically inactive effector protein). Accordingly, an effector protein as used herein encompasses a modified or effector protein that does not have nuclease activity.

The term “endonuclease activity” can refer to the enzymatic activity of an enzyme which allows the enzyme to cleave the phosphodiester bond within a polynucleotide chain.

The term, “engineered modification,” as used herein, refers to a structural change of one or more nucleic acid residues of a nucleotide sequence or one or more amino acid residue of an amino acid sequence, such as chemical modification of one or more nucleobases; or a chemical change to the phosphate backbone, a nucleotide, a nucleobase, or a nucleoside. Such modifications can be made to an effector protein amino acid sequence or guide nucleic acid nucleotide sequence, or any sequence disclosed herein (e.g., a nucleic acid encoding an effector protein or a nucleic acid that encodes a guide nucleic acid). Methods of modifying a nucleic acid or amino acid sequence are known. One of ordinary skill in the art will appreciate that the engineered modification(s) may be located at any position(s) of a nucleic acid such that the function of the nucleic acid, protein, composition, device, kit or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some instances, the nucleic acids provided herein are not uniformly modified along the entire length of the molecule. Different nucleotide modifications and/or backbone structures can exist at various positions within the nucleic acid.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “functional domain,” as used herein, refers to a region of one or more amino acids in a protein that is required for an activity of the protein, or the full extent of that activity, as measured in an in vitro assay. Activities include, but are not limited to nucleic acid binding, nucleic acid modification, nucleic acid cleavage, protein binding. The absence of the functional domain, including mutations of the functional domain, would abolish or reduce activity.

The term “fusion effector protein,” “fusion protein,” and “fusion polypeptide,” as used herein, refers to a protein comprising at least two heterologous polypeptides. Often a fusion effector protein comprises an effector protein and a fusion partner protein. In general, the fusion partner protein is not an effector protein. Examples of fusion partner proteins are provided herein.

The term “guide nucleic acid,” as used herein, refers to a nucleic acid molecule having: 1) a sequence of nucleotides that are sufficiently complementary to a sequence of nucleotides in a target nucleic acid to allow the nucleic acid molecule to hybridize to the target nucleic acid; and 2) a sequence of nucleotides that are sufficient for an effector protein to bind to the nucleic acid molecule. A guide nucleic acid, when complexed with an effector protein, can also direct binding of the effector protein a target nucleic acid. Sufficient conditions for hybridization of a guide nucleic acid to a target nucleic acid and/or for binding of a guide nucleic acid to an effector protein include in vivo physiological conditions of a desired cell type or in vitro conditions sufficient for assaying catalytic activity of a protein, polypeptide or peptide described herein, such as an effector protein. A guide nucleic acid can be DNA or RNA. When a guide nucleic acid is RNA, it can be referred to herein as a “gRNA.” Such a gRNA includes, but is not limited to, a crRNA or a crRNA in combination with an associated tracrRNA are attached (e.g., covalently) by an artificial linker. A gRNA may include deoxyribonucleotides and chemically modified nucleotides. A guide nucleic acid may include a naturally occurring guide nucleic acid or non-naturally occurring guide nucleic acid molecule, including a guide nucleic acid that is designed to contain a chemical or biochemical modification.

The term, “handle sequence,” as used herein, refers to a sequence of nucleotides in a single guide RNA (sgRNA), that is: 1) capable of being non-covalently bound by an effector protein and 2) connects the portion of the sgRNA capable of being non-covalently bound by an effector protein to a nucleotide sequence that is hybridizable to a target nucleic acid. In general, the handle sequence comprises an intermediary RNA sequence, that is capable of being non-covalently bound by an effector protein. In some instances, the handle sequence further comprises a repeat sequence. In such instances, the intermediary RNA sequence or a combination of the intermediary RNA and the repeat sequence is capable of being non-covalently bound by an effector protein.

The terms “heater”, “heating unit”, “heating element”, “heat source”, and the like, as used herein in reference to a device, generally refers to an element that is configured to produce heat and is in thermal communication with a portion of a device.

The term “heterologous,” as used herein, means a nucleotide or polypeptide sequence that is not found in the native nucleic acid or protein, respectively. In some instances, the fusion partner protein may be heterologous to the effector protein, and thus, referred to herein as a “heterologous protein.” A protein that is heterologous to the effector protein is a protein that is not covalently linked via an amide bond to the effector protein in nature. A heterologous protein may not be encoded by a species that encodes the effector protein. In some instances, the heterologous protein exhibits an activity (e.g., enzymatic activity) that it exhibits when it is fused to the effector protein. In some instances, the heterologous protein exhibits increased or reduced activity (e.g., enzymatic activity) when it is fused to the effector protein, relative to when it is not fused to the effector protein. In some instances, the heterologous protein exhibits an activity (e.g., enzymatic activity) that it does not exhibit when it is fused to the effector protein.

As used herein, “HotPot” refers to a one-pot reaction in which both amplification (e.g., RT-LAMP) and detection (e.g., DETECTR) reactions occur simultaneously. In many embodiments, a HotPot reaction may utilize a thermostable effector protein which exhibits trans cleavage at elevated temperatures (e.g., greater than 37C).

The term, “indel,” as used herein, refers to an insertion-deletion or indel mutation, which is a type of genetic mutation that results from the insertion and/or deletion of one or more nucleotide in a target nucleic acid. An indel can vary in length (e.g., 1 to 1,000 nucleotides in length) and be detected by any suitable method, including sequencing.

The term, “indel percentage,” as used herein, refers to a percentage of sequencing reads that show at least one nucleotide has been edited from the insertion and/or deletion of nucleotides regardless of the size of insertion or deletion, or number of nucleotides edited. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are edited by a given effector protein.

The terms, “intermediary RNA” and “intermediary sequence,” as used herein, in a context of a single nucleic acid system, refers to a nucleotide sequence in a handle sequence, wherein the nucleotide sequence is capable of, at least partially, being non-covalently bound to an effector protein to form a complex (e.g., an RNP complex). An intermediary sequence is not a transactivating nucleic acid in systems, methods, and compositions described herein.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The term “in vivo” is used to describe an event that takes place in a subject's body.

The term “linker,” as used herein, refers to a bond or molecule that links a first polypeptide to a second polypeptide. A “peptide linker” comprises at least two amino acids linked by an amide bond.

The term “linked amino acids” refers to at least two amino acids linked by an amide bond.

The term “modified target nucleic acid,” as used herein, refers to a target nucleic acid has undergone a change (e.g., chemical or physical). Such a change can be, for example, after contact with an effector protein. In some instances, the modification is an alteration in the sequence of the target nucleic acid. In some instances, the modified target nucleic acid comprises an insertion, deletion, or replacement of one or more nucleotides compared to the unmodified target nucleic acid.

The term “mutation associated with a disease,” as used herein, refers to the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

The terms “non-naturally occurring” or “engineered,” as used herein, are used interchangeably, and indicate the involvement of the hand of man. The terms, when referring to a nucleic acid molecule, nucleotide, protein, polypeptide, peptide or amino acid, refer to a nucleic acid molecule, nucleotide, protein, polypeptide, peptide or amino acid that is at least substantially free from at least one other component with which it is naturally associated in nature and as found in nature, or contains a modification (e.g., chemical modification, nucleotide sequence, or amino acid sequence) that is not present in the naturally occurring nucleic acid molecule, nucleotide, protein, polypeptide, peptide, amino acid. The terms, when referring to a composition or system described herein, refer to a composition or system having at least one component that is not naturally associated with the other components of the composition or system. By way of a non-limiting example, a composition can include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by a human or machine.

The term “nuclear localization signal” refers to an entity (e.g., peptide) that facilitates localization of a nucleic acid, protein, or small molecule to the nucleus, when present in a cell that contains a nuclear compartment.

The term “nuclease activity,” as used herein, refers to the enzymatic activity of an enzyme that allows the enzyme to cleave (hydrolyze) the phosphodiester bonds between the nucleotide subunits of a nucleic acid molecule. Nuclease activity can also be specified as endonuclease activity, which refers to the enzymatic activity of an enzyme that allows the enzyme to cleave the phosphodiester bond within the nucleic acid molecule, whereas exonuclease activity refers to the enzymatic activity of an enzyme that allows the enzyme to cleave the bond between nucleotides at the 3′ or 5′ ends of the nucleic acid molecule. An enzyme with nuclease activity may be referred to as a “nuclease.”

The term “nucleic acid expression vector,” as used herein, refers to a segment of nucleic acids (DNA or RNA) that allows expression (transcription and/or translation) of the inserted nucleotide sequence of interest. An expression vector can include a promoter (e.g., constitutive or inducible) or other regulatory element and a transcription termination sequence operably linked to the inserted nucleotide sequence of interest. An expression vector may also carry a ribosome binding sequence (for bacterial expression) and a start codon, depending on the nature of the inserted nucleotide sequence. An expression vector can be episomal (e.g., a plasmid) or integrated into the genome of a host organism.

The terms “nucleotide” and “nucleoside” when used in the context of a nucleic acid molecule having multiple residues are used interchangeably and mean the sugar and base of the residue contained in the nucleic acid molecule. The term “nucleobase” when used in the context of a nucleic acid molecule can refer to the base of the residue contained in the nucleic acid molecule, for example, the base of a nucleotide or a nucleoside.

As used herein, a “one-pot” reaction refers to a reaction in which more than one reaction occurs in a single volume alongside an effector protein-based detection (e.g., DETECTR) assay. For example, in a one-pot assay, sample preparation, reverse transcription, amplification, in vitro transcription, or any combination thereof, and effector protein-based detection (e.g., DETECTR) assays (optionally including signal amplification) are carried out in a single volume. In some embodiments, amplification and detection are carried out within a same volume or region of a device (e.g., within a detection region). Readout of the detection (e.g., DETECTR) assay may occur in the single volume or in a second volume. For example, the product of the one-pot DETECTR reaction (e.g., a cleaved detection moiety comprising an enzyme) may be transferred to another volume (e.g., a volume comprising an enzyme substrate) for signal generation and indirect detection of reporter cleavage by a sensor or detector (or by eye in the case of a colorimetric signal).

The term “PAM” or “protospacer adjacent motif,” as used herein, refers to a short nucleotide sequence found in a target nucleic acid molecule, such as a target DNA, that allows an effector protein to bind the target nucleic acid molecule and modify the target nucleic acid molecule at a specific location. A PAM can be specifically recognized and bound by an effector protein complexed with a guide nucleic acid and result in the effector protein modifying the target nucleic acid molecule (e.g., cleaving (hydrolysis of a phosphodiester bond) of a nucleic acid molecule resulting in a nick or double strand break-endonuclease activity) adjacent to the PAM. A given effector protein may or may not require a PAM being present in a target nucleic acid molecule for modifying the target nucleic acid molecule.

The term “pharmaceutically acceptable excipient, carrier or diluent,” as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

The terms “reagent mix,” “reagent master mix,” “reagents,” and the like, as used herein, generally refers to a formulation comprising one or more chemicals that partake in a reaction that the formulation is intended for

The term “recombinant,” as used herein, as applied to proteins, polypeptides, peptides and nucleic acids, can refer to proteins, polypeptides, peptides and nucleic acids that are products of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may act to modulate production of a desired product by various mechanisms. Thus, for example, the term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. Similarly, the term “recombinant polypeptide” or “recombinant protein” refers to one which is not naturally occurring, e.g., is made by the artificial combination of two otherwise separated segments of amino sequences through human intervention. Thus, for example, a polypeptide that includes a heterologous amino acid sequence is a recombinant polypeptide.

The term “reporter” and “reporter nucleic acid,” as used herein, refers generally to a non-target nucleic acid molecule that is capable of providing a detectable signal upon cleavage by an effector protein. Examples of detectable signals and detectable moieties that generate detectable signals are provided herein.

The terms, “ribonucleotide protein complex” and “RNP” as used herein, refer to a complex of one or more nucleic acids and one or more polypeptides described herein. While the term utilizes “ribonucleotides” it is understood that the one or more nucleic acid may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The term “sample,” as used herein, generally refers to something comprising a target nucleic acid. In some instances, the sample is a biological sample, such as a biological fluid or tissue sample. In some instances, the sample is an environmental sample. The sample may be a biological sample or environmental sample that is modified or manipulated. By way of non-limiting example, samples may be modified or manipulated with purification techniques, heat, nucleic acid amplification, salts, and buffers.

The terms “sample interface,” “sample input,” “input port,” “input” and “port,” as used herein in reference to a device, generally refers to a compartment that is configured to receive a sample, and optionally contain or hold a sample, for assaying purposes. The sample interface may be connected or communicative with the other components of the device for the assay (e.g., a detection reaction) to occur.

The term, “single nucleic acid system,” as used herein, refers to a system that uses a guide nucleic acid complexed with one or more polypeptides described herein, wherein the complex is capable of interacting with a target nucleic acid in a sequence specific manner, and wherein the guide nucleic acid is capable of non-covalently interacting with the one or more polypeptides described herein, and wherein the guide nucleic acid is capable of hybridizing with a target sequence of the target nucleic acid. A single nucleic acid system lacks a duplex of a guide nucleic acid as hybridized to a second nucleic acid, wherein in such a duplex the second nucleic acid, and not the guide nucleic acid, is capable of interacting with the effector protein. In a single nucleic system, the guide nucleic acid is not transactivating or transactivated. In a single nucleic acid system, the guide nucleic acid-polypeptide complex (e.g., an RNP complex) is not transactivated or transactivating.

The term “subject,” as used herein, refers a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some instances, the subject is not necessarily diagnosed or suspected of being at high risk for the disease.

The term, “sufficiently complementary,” as used herein, refers to a first nucleotide sequence that is partially complementarity to a second nucleotide sequence while still allowing the first nucleotide sequence to hybridize to the second nucleotide sequence with enough affinity to permit a biological activity to occur. Depending on the context, a biological activity may be the formation of a complex between two or more components described herein, such as an effector protein and a guide nucleic acid. A biological activity may also be bringing one or more components described herein into proximity of another component, such as bringing an effector protein-guide nucleic acid complex into proximity of a target nucleic acid. A biological activity may additionally be permitting a component described herein to act on another component described herein, such as permitting an effector protein to cleave a target nucleic acid. In some instances, sequences are said to be sufficiently complementary when at least 85% of the residues of a nucleotide sequence are complementary to residues in a reference nucleotide sequence.

A “syndrome”, as used herein, can refer to a group of symptoms which, taken together, characterize a condition.

The term “target nucleic acid,” as used herein, refers to a nucleic acid molecule that is selected as the nucleic acid molecule for modification, binding, hybridization, or any other activity of or interaction with a nucleic acid, protein, polypeptide, peptide described herein. A target nucleic acid can be RNA or DNA. A target nucleic acid can be single-stranded (e.g., single-stranded RNA or single-stranded DNA) or double-stranded (e.g., double-stranded DNA). The target nucleic acid can be from any organism, including, but not limited to, a bacterium, a virus, a parasite, a protozoon, a fungus, a mammal, a plant, and an insect. As another non-limiting example, the target nucleic acid can be responsible for a disease, contain a mutation (e.g., single strand polymorphism, point mutation, insertion, or deletion), be contained in an amplicon, or be uniquely identifiable from the surrounding nucleic acid molecules (e.g., contain a unique sequence of nucleotides). A target nucleic acid can have a “target sequence” that is complementary to a guide nucleic acid, where hybridization between the target nucleic acid and the guide nucleic acid promotes the association of an effector protein with the target nucleic acid.

As used herein, the terms “thermostable” and “thermostability” refer to the stability of a composition disclosed herein at one or more temperatures, such as an elevated operating temperature for a given reaction. Stability may be assessed by the ability of the composition to perform an activity, e.g., cleaving a target nucleic acid or reporter. Improving thermostability means improving the quantity or quality of the activity at one or more temperatures.

The term “threshold of detection” is used herein to describe the minimal amount of target nucleic acid that must be present in a sample in order for detection to occur.

The term “transactivating” or “transactivate,” as used herein, refers to the ability of a tracrRNA to (1) hybridize to a crRNA, wherein the tracrRNA and the crRNA are not covalently linked, and wherein the crRNA comprises a region that hybridizes to a target nucleic acid; and (2) interact with an effector protein, thereby bringing the effector protein into the proximity of the target nucleic acid where the effector protein provides a modifying activity on the target nucleic acid. In general, a tracrRNA is a feature of a dual-guide system.

The term “trans-activating crRNA” or “tracrRNA”, as used herein, an RNA molecule that serves as a binding scaffold for an effector protein that allows for association of the effector protein with a guide nucleic acid (e.g., crRNA). A tracrRNA can include deoxyribonucleosides in addition to ribonucleosides. A tracrRNA can be separate from, but form a complex with, a crRNA. The tracrRNA sequence may be attached (e.g., covalently) by an artificial linker to a crRNA to form an “sgRNA” or “single guide RNA.” A tracrRNA can also form a secondary structure (e.g., one or more hairpin loops) that facilitates the binding of an effector protein to a specific target nucleic acid. A tracrRNA can include a repeat hybridization region and a hairpin region. The repeat hybridization region can hybridize to all or part of the sequence of the repeat of a crRNA. The repeat hybridization region can be positioned 3′ of the hairpin region. The hairpin region can include a first sequence, a second sequence that is reverse complementary to the first sequence, and a stem-loop linking the first sequence and the second sequence.

As used herein, the term “trans cleavage” when used in reference to cleavage (hydrolysis of a phosphodiester bond) of a target nucleic acid by an effector protein complexed with a guide nucleic acid refers to cleavage of a target nucleic acid that is hybridized to a guide nucleic acid, wherein cleavage occurs near, but not within or directly adjacent to, the region of the target nucleic acid that is hybridized to the guide nucleic acid. trans cleavage activity can be triggered by the hybridization of the guide nucleic acid to the target nucleic acid.

The term “transcriptional activator,” as used herein, refers to a polypeptide or a fragment thereof that is capable of activating or increasing transcription of a target nucleic acid molecule.

The term “transcriptional repressor,” as used herein, refers to a polypeptide or a fragment thereof that is capable of arresting, preventing, or reducing transcription of a target nucleic acid.

The term, “transgene,” as used herein, refers to a nucleotide sequence that is inserted into a cell for expression of said nucleotide sequence in the cell. A transgene is meant to include (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. A donor nucleic acid can comprise a transgene. The cell in which transgene expression occurs can be a target cell, such as a host cell.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term, “valve,” as used herein, refers to a mechanism or device for directing, regulating, controlling, or obstructing the passage of fluid, gas, or loose materials through an opening or passageway. A valve may regulate the movement of fluid through an opening in one direction only. A valve may operate automatically, pneumatically, hydraulically, mechanically, electrically, chemically or combinations thereof.

As used herein, the term “viral vector” refers to a recombinantly produced virus or viral particle that includes a nucleic acid (DNA or RNA, single-stranded or double stranded, linear or circular, segmented or non-segmented) to be delivered into a host cell. Non-limiting examples of viral vectors include retroviral vectors (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector can be replication competent, replication deficient or replication defective.

### INTRODUCTION

Disclosed herein are compositions, systems, devices, kits and methods comprising at least one of:
a) a polypeptide or a nucleic acid encoding the polypeptide; and
b) a guide nucleic acid or a nucleic acid encoding the guide nucleic acid.

Polypeptides described herein may bind and, optionally, cleave nucleic acids in a sequence-specific manner. Polypeptides described herein may also cleave the target nucleic acid within a target sequence or at a position adjacent to the target sequence. In some embodiments, a polypeptide is activated when it binds a certain sequence of a nucleic acid described herein, allowing the polypeptide to cleave a region of a target nucleic acid that is near, but not adjacent to the target sequence. A polypeptide may be an effector protein, such as a CRISPR-associated (Cas) protein, which may bind a guide nucleic acid that imparts activity or sequence selectivity to the polypeptide. An effector protein may also be referred to as a programmable nuclease because the nuclease activity of the protein may be directed to different target nucleic acids by way of revising the guide nucleic acid that the protein binds.

Also disclosed herein are non-naturally occurring compositions, methods, devices, kits and systems comprising an effector protein and an engineered guide nucleic acid, which may simply be referred to herein as a guide nucleic acid. In some embodiments, compositions, systems, devices, kits and methods comprise a guide nucleic acid or a use thereof. In some embodiments, compositions, systems, devices, kits and methods comprise an engineered protein or a use thereof. In some embodiments, compositions, systems, devices, kits and methods comprise an isolated polypeptide or a use thereof.

In some embodiments, compositions, systems, devices, kits and methods comprising effector proteins and guide nucleic acids comprise a first sequence, at least a portion of which interacts with a polypeptide. In some embodiments, the first sequence comprises a sequence that is similar or identical to an intermediary nucleic acid sequence, a repeat sequence, or a combination thereof. In some embodiments, the guide nucleic acid does not comprise an intermediary nucleic acid. In some embodiments, compositions, systems, devices, kits and methods comprising effector proteins and guide nucleic acids comprise a second sequence that is at least partially complementary to a target nucleic acid, and which may be referred to as a spacer sequence.

Effector proteins disclosed herein may cleave nucleic acids, including single stranded RNA (ssRNA), double stranded DNA (dsDNA), and single-stranded DNA (ssDNA). Polypeptides disclosed herein may provide cis cleavage activity, trans cleavage activity, nickase activity, nuclease activity, or a combination thereof.

The compositions, systems, devices, kits and methods described herein are non-naturally occurring. In general, an engineered effector protein and an engineered guide nucleic acid refer to an effector protein and a guide nucleic acid, respectively, that are not found in nature. In some embodiments, methods, systems, devices, kits and compositions described herein comprise at least one non-naturally occurring component. For example, disclosed methods, compositions and systems may comprise a guide nucleic acid, wherein the sequence of the guide nucleic acid is different or modified from that of a naturally occurring guide nucleic acid. In some embodiments, methods, compositions, devices, kits and systems described herein comprise at least two components that do not naturally occur together. For example, disclosed methods, compositions, devices, kits and systems may comprise a guide nucleic acid comprising a repeat sequence and a spacer sequence which do not naturally occur together. Also, by way of example, disclosed methods, composition and systems may comprise a guide nucleic acid and an effector protein that do not naturally occur together. Likewise, by way of non-limiting example, disclosed compositions, systems, devices, kits and methods may comprise a ribonucleotide-protein (RNP) complex comprising an effector protein and a guide nucleic acid that do not occur together in nature. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some embodiments, the guide nucleic acid comprises a non-natural nucleotide sequence. In some embodiments, the non-natural sequence is a nucleotide sequence that is not found in nature. The non-natural nucleotide sequence may comprise a portion of a naturally occurring sequence, wherein the portion of the naturally occurring sequence is not present in nature, absent the remainder of the naturally occurring sequence. In some embodiments, the guide nucleic acid comprises two naturally occurring sequences arranged in an order or proximity that is not observed in nature. In some embodiments, compositions, devices, kits, methods and systems comprise a ribonucleotide complex comprising an effector protein and a guide nucleic acid that do not occur together in nature. Engineered guide nucleic acids may comprise a first sequence and a second sequence that do not occur naturally together. For example, an engineered guide nucleic acid may comprise a sequence of a naturally occurring repeat sequence, comprising a repeat sequence, and a spacer sequence, comprising a spacer sequence, that is complementary to a naturally occurring eukaryotic sequence. The engineered guide nucleic acid may comprise a sequence of a repeat sequence that occurs naturally in an organism and a spacer sequence that does not occur naturally in that organism. An engineered guide nucleic acid may comprise a first sequence that occurs in a first organism and a second sequence that occurs in a second organism, wherein the first organism and the second organism are different. The guide nucleic acid may comprise a third sequence located at a 3′ or 5′ end of the guide nucleic acid, or between the first and second sequences of the guide nucleic acid. For example, an engineered guide nucleic acid may comprise a naturally occurring CRISPR RNA (crRNA) and trans-activating crRNA (tracrRNA) sequence coupled by a linker sequence. In some embodiments, the guide nucleic acid comprises two heterologous sequences arranged in an order or proximity that is not observed in nature. Therefore, compositions, methods, devices, kits and systems described herein are not naturally occurring.

In some embodiments, compositions, methods, devices, kits and systems described herein comprise an engineered effector protein that is similar to a naturally occurring effector protein. The engineered effector protein (referred to herein as an engineered protein) may lack a portion of the naturally occurring effector protein. The effector protein may comprise a mutation relative to the naturally occurring effector protein, wherein the mutation is not found in nature. The effector protein may also comprise at least one additional amino acid relative to the naturally occurring effector protein. In some embodiments, the effector protein may comprise a heterologous polypeptide. For example, the effector protein may comprise an addition of a nuclear localization signal relative to the natural occurring effector protein. In certain embodiments, the nucleotide sequence encoding the effector protein is codon optimized (e.g., for expression in a eukaryotic cell) relative to the naturally occurring sequence.

### I. Polypeptide Systems

Provided herein are compositions, methods, devices, kits and systems that comprise one or more polypeptides or proteins, and/or uses thereof. A polypeptide or protein describes a polymeric form of amino acids. A polypeptide may include coded and non-coded amino acids, chemically or biochemically modified or derivatized amino acids, and polypeptides having modified peptide backbones. Accordingly, polypeptides as described herein may comprise one or more mutations, one or more engineered modifications, or both. It is understood that when describing coding sequences of polypeptides described herein, said coding sequences do not necessarily require a codon encoding an N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some embodiments, when a heterologous peptide, such as a fusion partner protein, protein tag or NLS, is located at the N terminus of the effector protein, a start codon for the heterologous peptide serves as a start codon for the effector protein as well. Thus, the natural start codon encoding an amino acid residue sufficient for initiating translation (e.g., Methionine (M) or a Valine (V)) of the effector protein may be removed or absent.

### Effector Proteins

Provided herein, in certain embodiments, are compositions, methods, devices, kits and systems that comprise one or more effector proteins and/or uses thereof.

An effector protein may be brought into proximity of a target nucleic acid in the presence of a guide nucleic acid when the guide nucleic acid includes a nucleotide sequence that is complementary with a target sequence in the target nucleic acid. An effector protein provided herein interacts with a guide nucleic acid to form a complex. In some embodiments, the effector protein non-covalently binds to a guide nucleic acid to form a complex that contacts a target nucleic acid, wherein at least a portion of the guide nucleic acid hybridizes to a target sequence of the target nucleic acid. In some embodiments, the complex interacts with a target nucleic acid, a non-target nucleic acid, or both. In some embodiments, an interaction between the complex and a target nucleic acid, a non-target nucleic acid, or both comprises one or more of: recognition of a protospacer adjacent motif (PAM) sequence within the target nucleic acid by the effector protein, hybridization of the guide nucleic acid to the target nucleic acid, modification of the target nucleic acid and/or the non-target nucleic acid by the effector protein, or combinations thereof.

A complex between an effector protein and a guide nucleic acid can include multiple effector proteins or a single effector protein. In some embodiments, a complex having two or more effector proteins can include two or more of the same effector proteins (e.g., dimer or multimer).

In some embodiments, the effector protein modifies the target nucleic acid when the complex contacts the target nucleic acid. In some embodiments, the effector protein does not modify the target nucleic acid, but it is fused to a fusion partner protein that modifies the target nucleic acid when the complex contacts the target nucleic acid. In some embodiments, an effector protein as used herein encompasses a modified or effector protein that does not have modification activity. An effector protein may also recognize a protospacer adjacent motif (PAM) sequence present in the target nucleic acid, which may direct the modification activity of the effector protein.

Modification activity of an effector protein or an engineered protein described herein may be cleavage activity, binding activity, insertion activity, substitution activity, and the like. Modification activity of an effector protein may result in: cleavage of at least one strand of a target nucleic acid, deletion of one or more nucleotides of a target nucleic acid, insertion of one or more nucleotides into a target nucleic acid, substitution of one or more nucleotides of a target nucleic acid with an alternative nucleotide, more than one of the foregoing, or any combination thereof. In some embodiments, an ability of an effector protein to edit a target nucleic acid may depend upon the effector protein being complexed with a guide nucleic acid, the guide nucleic acid being hybridized to a target sequence of the target nucleic acid, the distance between the target sequence and a PAM sequence, or combinations thereof. A target nucleic acid comprises a target strand and a non-target strand. Accordingly, in some embodiments, the effector protein may edit a target strand and/or a non-target strand of a target nucleic acid.

The modification of the target nucleic acid generated by an effector protein may, as a non-limiting example, result in modulation of the expression of the target nucleic acid (e.g., increasing or decreasing expression of the nucleic acid) or modulation of the activity of a translation product of the target nucleic acid (e.g., inactivation of a protein binding to an RNA molecule or hybridization). Accordingly, in some embodiments, provided herein are methods of editing a target nucleic acid using an effector protein of the present disclosure, or compositions, devices, kits or systems thereof. Also provided herein are methods of modulating expression of a target nucleic acid using an effector protein of the present disclosure, or compositions, devices, kits or systems thereof. Further provided herein are methods of modulating the activity of a translation product of a target nucleic acid using an effector protein of the present disclosure, or compositions, devices, kits or systems thereof.

In some embodiments, effector proteins disclosed herein may provide cleavage activity, such as cis cleavage activity, trans cleavage activity, nickase activity, nuclease activity, or a combination thereof. In general, effector proteins described herein edit a target nucleic acid by cis cleavage activity on the target nucleic acid. In general, effector proteins described herein edit a target nucleic acid by trans cleavage activity on the target nucleic acid. Effector proteins disclosed herein may cleave nucleic acids, including single stranded RNA (ssRNA), double stranded DNA (dsDNA), and single-stranded DNA (ssDNA).

In some embodiments, effector proteins catalyze cleavage of a target nucleic acid in a cell or a sample. In some embodiments, the target nucleic acid is single stranded (ss). In some embodiments, the target nucleic acid is double stranded (ds). In some embodiments, the target nucleic acid is dsDNA. In some embodiments, the target nucleic acid is ssDNA. In some embodiments, the target nucleic acid is RNA. In some embodiments, effector proteins cleave the target nucleic acid within a target sequence of the target nucleic acid. In some embodiments, effector proteins cleave the target nucleic acid, as well as additional nucleic acids in the cell or the sample, which may be referred to as trans cleavage activity or simply trans cleavage activity. In some embodiments, effector proteins catalyze cis cleavage activity. In some embodiments, effector proteins cleave both strands of dsDNA. A non-limiting example of an effector protein modifying a target nucleic acid is cleaving of a phosphodiester bond of the target nucleic acid. An effector protein may modify a nucleic acid by cis cleavage or trans cleavage. Additional examples are as described above and throughout.

An effector protein may be a CRISPR-associated (“Cas”) protein. An effector protein may function as a single protein, including a single protein that is capable of binding to a guide nucleic acid and editing a target nucleic acid. Alternatively, an effector protein may function as part of a multiprotein complex, including, for example, a complex having two or more effector proteins, including two or more of the same effector proteins (e.g., dimer or multimer). An effector protein, when functioning in a multiprotein complex, may have only one functional activity (e.g., binding to a guide nucleic acid), while other effector proteins present in the multiprotein complex are capable of the other functional activity (e.g., editing a target nucleic acid). In some embodiments, an effector protein, when functioning in a multiprotein complex, may have differing and/or complementary functional activity to other effector proteins in the multiprotein complex. Multimeric complexes, and functions thereof, are described in further detail below. An effector protein may be a modified effector protein having increased modification activity and/or increased substrate binding activity (e.g., substrate selectivity, specificity, and/or affinity). Alternatively, or in addition, an effector protein may be a catalytically inactive effector protein having reduced modification activity or no modification activity.

In some embodiments, effector proteins comprise a functional domain. The functional domain may comprise nucleic acid binding activity. The functional domain may comprise catalytic activity, also referred to as enzymatic activity. The catalytic activity may be nuclease activity. The nuclease activity may comprise cleaving a strand of a nucleic acid. The nuclease activity may comprise cleaving only one strand of a double stranded nucleic acid, also referred to as nicking. In some embodiments, the functional domain is an HNH domain. In some embodiments, the functional domain is a RuvC domain. In some embodiments, the RuvC domain comprises multiple subdomains. In some embodiments, the functional domain is a zinc finger binding domain. In some embodiments, the functional domain is a HEPN domain. In some embodiments, effector proteins lack a certain functional domain. In some embodiments, the effector protein lacks an HNH domain. In some embodiments, effector proteins lack a zinc finger binding domain. The nuclease activity can be endonuclease activity.

Also provided herein are compositions, devices, kits, methods and systems that comprise a nucleic acid, wherein the nucleic acid encodes any of one the effector proteins described herein. The nucleic acid may be a nucleic acid expression vector. By way of non-limiting example, the nucleic acid expression vector may be contained within a viral vector, such as an AAV vector. In another example, the one or more effector proteins and/or the expression vector may be contained in a lipid vector or a lipid particle.

TABLE 1 provides illustrative amino acid sequences of effector proteins that are useful in the compositions, systems, devices, kits and methods described herein. In some embodiments, the effector protein comprises an amino acid sequence that is at least 65%, at least 70%, at least 75%, at least 80%, at least 85%, at least 90%, at least 95%, at least 97%, or at least 98%, at least 99%, or 100% identical to any one of the sequences set forth in TABLE 1, e.g., any one of the sequences set forth in SEQ ID NOs: 1-28, or in SEQ ID NOs: 93-142. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences set forth in TABLE 1, e.g., any one of the sequences set forth in SEQ ID NOs: 1-28, or in SEQ ID NOs: 93-142. In some embodiments, the amino acid sequence of the effector protein is at least 80%, at least 85%, at least 90%, at least 95%, at least 97%, or at least 98%, or at least 99% similar to any one of the sequences recited in TABLE 1.

In some embodiments, compositions, systems, devices, kits and methods described herein comprise an effector protein, or a nucleic acid encoding the effector protein, wherein effector protein comprises an amino acid sequence that is at least 65%, at least 70%, at least 75%, at least 80%, at least 85%, at least 90%, at least 95%, at least 97%, or at least 98%, at least 99%, or 100% identical to any one of the sequences set forth in TABLE 1. In some embodiments, the amino acid sequence is at least 80% identical to any one of the sequences set forth in TABLE 1. In some embodiments, the amino acid sequence is at least 85% identical to any one of the sequences set forth in TABLE 1. In some embodiments, the amino acid sequence is at least 90% identical to any one of the sequences set forth in TABLE 1. In some embodiments, the amino acid sequence is at least 95% identical to any one of the sequences set forth in TABLE 1. In some embodiments, the amino acid sequence is at least 97% identical to any one of the sequences set forth in TABLE 1. In some embodiments, the amino acid sequence is at least 99% identical to any one of the sequences set forth in TABLE 1. In some embodiments, the amino acid sequence is 100% identical to any one of the sequences set forth in TABLE 1.

In some embodiments, compositions, systems, devices, kits and methods described herein comprise an effector protein, or a nucleic acid encoding the effector protein, wherein the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences set forth in TABLE 1. In some embodiments, the amino acid sequence of the effector protein is at least 80% identical to any one of the sequences set forth in TABLE 1. In some embodiments, the amino acid sequence of the effector protein is at least 85% identical to any one of the sequences set forth in TABLE 1. In some embodiments, the amino acid sequence of the effector protein is at least 90% identical to any one of the sequences set forth in TABLE 1. In some embodiments, the amino acid sequence of the effector protein is at least 95% identical to any one of the sequences set forth in TABLE 1. In some embodiments, the amino acid sequence of the effector protein is at least 97% identical to any one of the sequences set forth in TABLE 1. In some embodiments, the amino acid sequence of the effector protein is at least 99% identical to any one of the sequences set forth in TABLE 1. In some embodiments, the amino acid sequence of the effector protein is 100% identical to any one of the sequences set forth in TABLE 1.

In some embodiments, compositions, systems, devices, kits and methods described herein comprise an effector protein, or a nucleic acid encoding the effector protein, wherein a portion of the amino acid sequence of the effector protein is at least 65%, at least 70%, at least 75%, at least 80%, at least 85%, at least 90%, at least 95%, at least 97%, or at least 98%, at least 99%, or 100% identical to an equal length portion of a sequence selected from any one of the sequences set forth in TABLE 1. In some embodiments, the length of the portion is selected from: 20 to 40, 40 to 60, 60 to 80, 80 to 100, 100 to 120, 120 to 140, 140 to 160, 160 to 180, 180 to 200, 200 to 220, 220 to 240, 240 to 260, 260 to 280, 280 to 300, 320 to 340, 340 to 360, 360 to 380, and 380 to 400 linked amino acids. In some embodiments, the length of the portion is selected from: 400 to 420, 420 to 440, 440 to 460, 460 to 480, 480 to 500, 520 to 540, 540 to 560, 560 to 580, 580 to 600, 600 to 620, 620 to 640, 640 to 660, 660 to 680, and 680 to 700, 700 to 720, 720 to 740, 740 to 760, 760 to 780, 780 to 800, 820 to 840, 840 to 860, 860 to 880, 880 to 900, 900 to 920, 920 to 940, 940 to 960, 960 to 980, and 980 to 1000. In some embodiments, the length of the portion is selected from: 1000 to 1020, 1020 to 1040, 1040 to 1060, 1060 to 1080, 1080 to 1100, 1100 to 1120, 1120 to 1140, 1140 to 1160, 1160 to 1180, 1180 to 1200, 1220 to 1240, 1240 to 1260, 1260 to 1280, 1280 to 1300, 1300 to 1320, and 1320 to 1340.

In some embodiments, compositions, systems, devices, kits and methods described herein comprise an effector protein or a nucleic acid encoding the effector protein, wherein the effector protein comprises an amino acid sequence that is at least 80%, at least 85%, at least 90%, at least 95%, at least 97%, or at least 98%, or at least 99% similar to any one of the sequences recited in TABLE 1. In some embodiments, the amino acid sequence of the effector protein is at least 80% similar to any one of the sequences set forth in TABLE 1. In some embodiments, the amino acid sequence of the effector protein is at least 85% similar to any one of the sequences set forth in TABLE 1. In some embodiments, the amino acid sequence of the effector protein is at least 90% similar to any one of the sequences set forth in TABLE 1. In some embodiments, the amino acid sequence of the effector protein is at least 95% similar to any one of the sequences set forth in TABLE 1. In some embodiments, the amino acid sequence of the effector protein is at least 97% similar to any one of the sequences set forth in TABLE 1. In some embodiments, the amino acid sequence of the effector protein is at least 99% similar to any one of the sequences set forth in TABLE 1. In some embodiments, the amino acid sequence of the effector protein is 100% similar to any one of the sequences set forth in TABLE 1.

In some embodiments, compositions, systems, devices, kits and methods described herein comprise an effector protein or a nucleic acid encoding the effector protein, wherein the effector protein comprises a portion of any one of the sequences recited in TABLE 1. In some embodiments, the effector protein comprises a portion of any one of the sequences recited in TABLE 1, wherein the portion does not comprise at least the first 10 amino acids, first 20 amino acids, 40 amino acids, 60 amino acids, 80 amino acids, 100 amino acids, 120 amino acids, 140 amino acids, 160 amino acids, 180 amino acids, or 200 amino acids of any one of the sequences recited in TABLE 1. In some embodiments, the effector protein comprises a portion of any one of the sequences recited in TABLE 1, wherein the portion does not comprise the last 10 amino acids, the last 20 amino acids, 40 amino acids, 60 amino acids, 80 amino acids, 100 amino acids, 120 amino acids, 140 amino acids, 160 amino acids, 180 amino acids, or 200 amino acids of any one of the sequences recited in TABLE 1.

In some embodiments, compositions, systems, devices, kits and methods described herein comprise an effector protein, or a nucleic acid encoding the effector protein, wherein the amino acid sequence of the effector protein comprises at least about 200 contiguous amino acids or more of any one of the sequences recited in TABLE 1. In some embodiments, the amino acid sequence of an effector protein provided herein comprises at least about 200, at least about 220, at least about 240, at least about 260, at least about 280, at least about 300, at least about 320, at least about 340, at least about 360, at least about 380, at least about 400 contiguous amino acids, at least about 420 contiguous amino acids, at least about 440 contiguous amino acids, at least about 460 contiguous amino acids, at least about 480 contiguous amino acids, at least about 500 contiguous amino acids, at least about 520 contiguous amino acids, at least about 540 contiguous amino acids, at least about 560 contiguous amino acids, at least about 580 contiguous amino acids, at least about 600 contiguous amino acids, at least about 620 contiguous amino acids, at least about 640 contiguous amino acids, at least about 660 contiguous amino acids, at least about 680 contiguous amino acids, at least about 700 contiguous amino acids, or more of any one of the sequences of TABLE 1.

In some embodiments, compositions, systems, and methods described herein comprise an effector protein, or a nucleic acid encoding the effector protein, wherein the effector protein comprises one or more amino acid alterations relative to any one of the sequences recited in TABLE 1. In some embodiments, the effector protein comprising one or more amino acid alterations is a variant of an effector protein described herein. It is understood that any reference to an effector protein herein also refers to an effector protein variant as described herein. In some embodiments, the one or more amino acid alterations comprises conservative substitutions, non-conservative substitutions, conservative deletions, non-conservative deletions, or combinations thereof. In some embodiments, an effector protein or a nucleic acid encoding the effector protein comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to any one of the sequences recited in TABLE 1.

A conservative substitution describes the replacement of one amino acid for another such that the replacement takes place within a family of amino acids that are related in their side chains. Conversely, the term “non-conservative substitution” as used herein refers to the replacement of one amino acid residue for another that does not have a related side chain. Genetically encoded amino acids can be divided into four families having related side chains: (1) acidic (negatively charged): Asp (D), Glu (E); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser(S), Thr (T). Amino acids may be related by aliphatic side chains: Gly (G), Ala (A), Val (V), Leu (L), Ile (I), Ser(S), Thr (T), with Ser(S) and Thr (T) optionally being grouped separately as aliphatic-hydroxyl; Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M).

In some embodiments, the one or more amino acid alterations may result in a change in activity of the effector protein relative to a naturally-occurring counterpart. For example, and as described in further detail below, the one or more amino acid alteration increases or decreases catalytic activity of the effector protein relative to a naturally-occurring counterpart. In some embodiments, the one or more amino acid alterations results in a catalytically inactive effector protein variant.

### Engineered Proteins

In some embodiments, effector proteins described herein have been modified (also referred to as an engineered protein). In some embodiments, effector proteins disclosed herein are engineered proteins. Engineered proteins are not identical to a naturally occurring protein. Engineered proteins may not comprise an amino acid sequence that is identical to that of a naturally occurring protein. In some embodiments, the amino acid sequence of an engineered protein is not identical to that of a naturally occurring protein. In some embodiments, a modification of the effector proteins may include addition of one or more amino acids, deletion of one or more amino acids, substitution of one or more amino acids, or combinations thereof. In some embodiments, effector proteins disclosed herein are engineered proteins. Unless otherwise indicated, reference to effector proteins throughout the present disclosure include engineered proteins thereof.

In some embodiments, effector proteins described herein can be modified with the addition of one or more heterologous peptides or heterologous polypeptides (referred to collectively herein as a heterologous polypeptide). In some embodiments, an effector protein modified with the addition of one or more heterologous peptides or heterologous polypeptides may be referred to herein as a fusion protein. Such fusion proteins are described herein and throughout.

In some embodiments, a heterologous peptide or heterologous polypeptide comprises a subcellular localization signal. In some embodiments, a subcellular localization signal can be a nuclear localization signal (NLS). In some embodiments, the NLS facilitates localization of a nucleic acid, protein, or small molecule to the nucleus, when present in a cell that contains a nuclear compartment. TABLE 2 lists exemplary NLS sequences. An effector protein disclosed herein or fusion effector protein may comprise a nuclear localization signal (NLS). The NLS may be located at a variety of locations, including, but not limited to 5′ of the effector protein, 5′ of the fusion partner, 3′ of the effector protein, 3′ of the fusion partner, between the effector protein and the fusion partner, within the fusion partner, within the effector protein.

In some embodiments, the subcellular localization signal is a nuclear export signal (NES), a sequence to keep an effector protein retained in the cytoplasm, a mitochondrial localization signal for targeting to the mitochondria, a chloroplast localization signal for targeting to a chloroplast, an ER retention signal, and the like. In some embodiments, an effector protein described herein is not modified with a subcellular localization signal so that the polypeptide is not targeted to the nucleus, which can be advantageous depending on the circumstance (e.g., when the target nucleic acid is an RNA that is present in the cytosol).

In some embodiments, a heterologous peptide or heterologous polypeptide comprises a chloroplast transit peptide (CTP), also referred to as a chloroplast localization signal or a plastid transit peptide, which targets the effector protein to a chloroplast. Chromosomal transgenes from bacterial sources may require a sequence encoding a CTP sequence fused to a sequence encoding an expressed protein (e.g., the effector protein) if the expressed protein is to be compartmentalized in the plant plastid (e.g., chloroplast). The CTP may be removed in a processing step during translocation into the plastid. Accordingly, localization of an effector protein to a chloroplast is often accomplished by means of operably linking a polynucleotide sequence encoding a CTP sequence to the 5′ region of a polynucleotide encoding the exogenous protein.

In some embodiments, the heterologous polypeptide is an endosomal escape peptide (EEP). An EEP is an agent that quickly disrupts the endosome in order to minimize the amount of time that a delivered molecule, such an effector protein, spends in the endosome-like environment, and to avoid getting trapped in the endosomal vesicles and degraded in the lysosomal compartment. An exemplary EEP is set forth in TABLE 2.

In some embodiments, the heterologous polypeptide is a cell penetrating peptide (CPP), also known as a Protein Transduction Domain (PTD). A CPP or PTD is a polypeptide, polynucleotide, carbohydrate, or organic or inorganic compound that facilitates traversing a lipid bilayer, micelle, cell membrane, organelle membrane, or vesicle membrane.

Further suitable heterologous polypeptides include, but are not limited to, proteins (or fragments/domains thereof) that are boundary elements (e.g., CTCF), proteins and fragments thereof that provide periphery recruitment (e.g., Lamin A, Lamin B, etc.), and protein docking elements (e.g., FKBP/FRB, Pil1/Aby1, etc.).

In some embodiments, a heterologous peptide or heterologous polypeptide comprises a protein tag. In some embodiments, the protein tag is referred to as purification tag or a fluorescent protein. The protein tag may be detectable for use in detection of the effector protein and/or purification of the effector protein. Accordingly, in some embodiments, compositions, systems, devices, kits and methods comprise a protein tag or use thereof. Any suitable protein tag may be used depending on the purpose of its use. Non-limiting examples of protein tags include a fluorescent protein, a histidine tag, e.g., a 6XHis tag; a hemagglutinin (HA) tag; a FLAG tag; a Myc tag; and maltose binding protein (MBP). In some embodiments, the protein tag is a portion of MBP that can be detected and/or purified. Non-limiting examples of fluorescent proteins include green fluorescent protein (GFP), yellow fluorescent protein (YFP), red fluorescent protein (RFP), cyan fluorescent protein (CFP), mCherry, and tdTomato.

A heterologous polypeptide may be located at or near the amino terminus (N-terminus) of the effector protein disclosed herein. A heterologous polypeptide may be located at or near the carboxy terminus (C-terminus) of the effector proteins disclosed herein. In some embodiments, a heterologous polypeptide is located internally in an effector protein described herein (i.e., is not at the N- or C-terminus of an effector protein described herein) at a suitable insertion site.

In some embodiments, an effector protein described herein comprises 1, 2, 3, 4, 5, 6, 7, 8, 9, 10, or more heterologous polypeptides at or near the N-terminus, 1, 2, 3, 4, 5, 6, 7, 8, 9, 10, or more heterologous polypeptides at or near the C-terminus, or a combination of these (e.g., one or more heterologous polypeptides at the amino-terminus and one or more heterologous polypeptides at the carboxy terminus). When more than one heterologous polypeptide is present, each may be selected independently of the others, such that a single heterologous polypeptide may be present in more than one copy and/or in combination with one or more other heterologous polypeptides present in one or more copies. In some embodiments, a heterologous polypeptide is considered near the N- or C-terminus when the nearest amino acid of the heterologous polypeptide is within about 1, 2, 3, 4, 5, 10, 15, 20, 25, 30, 40, 50, or more amino acids along the polypeptide chain from the N- or C-terminus.

In some embodiments, a heterologous polypeptide described herein comprises a heterologous polypeptide sequence recited in TABLE 2. In some embodiments, effector proteins described herein comprise an amino acid sequence that is at least about 65%, at least about 70%, at least about 75%, at least about 80%, at least about 85%, at least about 90%, at least about 95%, at least about 97%, at least about 98%, at least about 99%, or about 100% identical to any one of the sequences recited in TABLE 1 and further comprises one or more of the sequences set forth in TABLE 2. In some embodiments, a heterologous peptide described herein may be a fusion partner as described en supra.

In some embodiments, effector proteins described herein are encoded by a codon optimized nucleic acid. In some embodiments, a nucleic acid sequence encoding an effector protein described herein, is codon optimized. An effector protein may be codon optimized for expression in a specific cell, for example, a bacterial cell, a plant cell, a eukaryotic cell, an animal cell, a mammalian cell, or a human cell. In some embodiments, the effector protein is codon optimized for a human cell.

A codon optimized polypeptide describes a mutation of a nucleotide sequence encoding a polypeptide, such as a nucleotide sequence encoding an effector protein, to mimic the codon preferences of the intended host organism or cell while encoding the same polypeptide. Thus, the codons can be changed, but the encoded polypeptide remains unchanged. For example, if the intended target cell was a human cell, a human codon-optimized nucleotide sequence encoding an effector protein could be used. As another non-limiting example, if the intended host cell were a mouse cell, then a mouse codon-optimized nucleotide sequence encoding an effector protein could be generated. As another non-limiting example, if the intended host cell were a eukaryotic cell, then a eukaryote codon-optimized nucleotide sequence encoding an effector protein could be generated. As another non-limiting example, if the intended host cell were a prokaryotic cell, then a prokaryote codon-optimized nucleotide sequence encoding an effector protein could be generated. Codon usage tables are readily available, for example, at the “Codon Usage Database” available at www.kazusa.or.jp/codon.

In some embodiments, compositions, systems, devices, kits and methods described herein comprise an engineered protein, or a nucleic acid encoding the engineered protein, wherein the engineered protein comprises one or more amino acid differences relative to any one of the sequences recited in TABLE 1. In some embodiments, the engineered protein comprising one or more amino acid differences is a variant of an effector protein described herein. It is understood that any reference to an effector protein or engineered protein herein also refers to an effector protein variant as described herein. In some embodiments, the amino acid sequence of an engineered protein comprises at least one residue that is different from that of a naturally occurring protein. In some embodiments, the amino acid sequence of an engineered protein comprises at least 2, at least 3, at least 4, at least 5, at least 6, at least 7, at least 8, at least 9 or at least 10 residues that are different from that of a naturally occurring protein. The residues in the engineered protein that differ from those at corresponding positions of the naturally occurring protein (when the engineered and naturally occurring proteins are aligned for maximal identity) may be referred to as substituted residues or amino acid substitutions. Alternative differences include deletions, additions, and combinations thereof. In some embodiments, the one or more amino acid differences comprises conservative substitutions, non-conservative substitutions, conservative deletions, non-conservative deletions, or combinations thereof. In some embodiments, the substituted residues are non-conserved residues relative to the residues at corresponding positions of the naturally occurring protein. A non-conserved residue has a different physicochemical property from the amino acid for which it substitutes. Physicochemical properties include aliphatic, cyclic, aromatic, basic, acidic and hydroxyl-containing amino acid. Glycine, alanine, valine, leucine, and isoleucine are aliphatic amino acids. Serine, Cysteine, threonine, and methionine are hydroxyl-containing. Proline is a cyclic amino acid. Phenylalanine, tyrosine, and tryptophan are basic amino acids. Aspartate, Glutamate, Asparagine, and glutamine are acidic amino acids. Conservative and non-conservative amino acid differences (e.g., substitutions) are further described herein.

In some embodiments, the one or more amino acid differences may result in a change in activity of the effector protein relative to a naturally-occurring counterpart. For example, and as described in further detail below, the one or more amino acid difference increases or decreases catalytic activity of the effector protein relative to a naturally-occurring counterpart. In some embodiments, the one or more amino acid difference results in a catalytically inactive effector protein variant.

In some embodiments, engineered proteins are designed to be catalytically inactive or to have reduced catalytic activity relative to a naturally occurring protein. A catalytically inactive effector protein can refer to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some embodiments, the catalytically inactive effector protein is referred to as a catalytically inactive variant of an effector protein, e.g., a Cas effector protein.

A catalytically inactive effector protein may be generated by substituting an amino acid that confers a catalytic activity (also referred to as a “catalytic residue”) with a substituted residue that does not support the catalytic activity. In some embodiments, the substituted residue has an aliphatic side chain. In some embodiments, the substituted residue is glycine. In some embodiments, the substituted residue is valine. In some embodiments, the substituted residue is leucine. In some embodiments, the substituted residue is alanine. In some embodiments, the amino acid is aspartate, and it is substituted with asparagine. In some embodiments, the amino acid is glutamate, and it is substituted with glutamine. An amino acid that confers catalytic activity may be identified by performing sequence alignment of an unmodified effector protein with a similar enzyme having at least one identified catalytic residue; selecting at least one putative catalytic residue in the unmodified effector protein within the portion of the unmodified effector protein that aligns with a portion of the similar enzyme that comprises the identified catalytic residue; substituting the at least one putative catalytic residue of the unmodified effector protein with the different amino acid; and comparing the catalytic activity of the unmodified effector protein to the modified effector protein. A similar enzyme may be an enzyme that is at least 50%, at least 60%, at least 70%, at least 80%, or at least 90% identical to the unmodified effector protein. A similar enzyme may be an enzyme that is not greater than 99.9% identical to the unmodified effector protein. In some embodiments, the portion of the unmodified effector protein that aligns with a portion of the similar enzyme is at least 10 amino acids, at least 20 amino acids, at least 30 amino acids, at least 40 amino acids, at least 50 amino acids, at least 60 amino acids, at least 70 amino acids, at least 80 amino acids, at least 90 amino acids, or at least 100 amino acids in length. In some embodiments, the portion of the unmodified effector protein that aligns with a portion of the similar enzyme is not greater than 200 amino acids. In some embodiments, the portion of the unmodified effector protein that aligns with a portion of the similar enzyme comprises a functional domain (e.g., HEPN, HNH, RuvC, zinc finger binding). In some embodiments, comparing the catalytic activity comprises performing a cleavage assay. An example of generating a catalytically inactive effector protein is provided in Example 7.

### Fusion Proteins

In some embodiments, compositions, devices, kits, methods and systems described herein comprise a fusion effector protein, wherein the fusion effector protein comprises an effector protein described herein. In some embodiments, compositions, devices, kits, methods and systems described herein comprise a nucleic acid encoding the fusion effector protein. In general, fusion effector proteins comprise an effector protein or a portion thereof, and a fusion partner protein. A fusion partner protein may also simply be referred to herein as a fusion partner. The terms “fusion partner protein” or “fusion partner,” as used herein, can refer to a protein, polypeptide or peptide that is fused to an effector protein.

In some embodiments, the fusion partner protein is fused to the N-terminus of the effector protein. In some embodiments, the fusion partner protein is fused to the C-terminus of the effector protein. In some embodiments, the amino terminus of the fusion partner is linked/fused to the carboxy terminus of the effector protein. In some embodiments, the carboxy terminus of the fusion partner protein is linked/fused to the amino terminus of the effector protein by the linker. In some embodiments, the effector protein is located at an internal location of the fusion partner protein. In some embodiments, the fusion partner protein is located at an internal location of the Cas effector protein. For example, a base editing enzyme (e.g., a deaminase enzyme) is inserted at an internal location of a Cas effector protein. The effector protein may be fused directly or indirectly (e.g., via a linker) to the fusion partner protein. Exemplary linkers are described herein.

In some embodiments, compositions, devices, kits, methods and systems described herein comprise a fusion effector protein and a guide nucleic acid, wherein at least a portion of the guide nucleic acid hybridizes to a target nucleic acid, and the fusion partner modulates the target nucleic acid or expression thereof. In general, the effector protein and the fusion partner protein are heterologous proteins. In some embodiments, fusion proteins comprise an effector protein and a fusion partner protein, wherein the fusion partner protein is heterologous to an effector protein. These fusion proteins may be referred to as a “heterologous protein.”

In some embodiments, the fusion partner is not an effector protein as described herein. In some embodiments, a fusion partner comprises a second effector protein, or a multimeric form thereof. Accordingly, in some embodiments, a fusion protein comprises more tan one effector protein. In such embodiments, the fusion protein can comprise at least two effector protein that are the same. In some embodiments, the fusion protein can comprise at least two effector protein that are different. In some embodiments, the multimeric form is a homomeric form. In some embodiments, the multimeric form is a heteromeric form. Unless otherwise indicated, reference to effector proteins throughout the present disclosure include fusion proteins comprising the effector protein described herein and a fusion partner.

The fusion partner generally imparts some function to the fusion protein that is not provided by the effector protein. Such activities may include but are not limited to nuclease activity, methyltransferase activity, demethylase activity, DNA repair activity, DNA damage activity, deamination activity, dismutase activity, alkylation activity, depurination activity, oxidation activity, dimer forming activity (e.g., pyrimidine dimer forming activity), integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity, modification of a polypeptide associated with target nucleic acid (e.g., a histone), and/or signaling activity.

In some embodiments, a fusion partner may provide signaling activity. The fusion partner may provide a detectable signal. In some embodiments, a fusion partner may inhibit or promote the formation of multimeric complex of an effector protein. The fusion partner may modify a target nucleic acid, including changing a nucleobase of the target nucleic acid and making a chemical modification to one or more nucleotides of the target nucleic acid. In an additional example, the fusion partner may directly or indirectly edit a target nucleic acid. In some embodiments, a fusion partner may modulate transcription (e.g., inhibits transcription, increases transcription) of a target nucleic acid. The fusion partner may be capable of modulating the expression of a target nucleic acid. In another example, a fusion partner may directly or indirectly inhibit, reduce, activate or increase expression of a target nucleic acid. The fusion partner may inhibit, reduce, activate or increase expression of a target nucleic acid via additional proteins or nucleic acid modifications to the target sequence. In some embodiments, the fusion partner may interact with additional proteins, or functional fragments thereof, to make modifications to a target nucleic acid. In other embodiments, the fusion partner may modify proteins associated with a target nucleic acid.

In some embodiments, fusion effector proteins modify a target nucleic acid or the expression thereof. In some embodiments, the modifications are transient (e.g., transcription repression or activation). In some embodiments, the modifications are inheritable. For embodiment, epigenetic modifications made to a target nucleic acid, or to proteins associated with the target nucleic acid, e.g., nucleosomal histones, in a cell, are observed in cells produced by proliferation of the cell.

### Multimeric Complex Formation Modification Activity

In some embodiments, a fusion partner may inhibit the formation of a multimeric complex of an effector protein. Alternatively, the fusion partner promotes the formation of a multimeric complex of the effector protein. By way of non-limiting example, the fusion protein may comprise an effector protein described herein and a fusion partner comprising a Calcineurin A tag, wherein the fusion protein dimerizes in the presence of Tacrolimus (FK506). Also, by way of non-limiting example, the fusion protein may comprise an effector protein described herein and a SpyTag configured to dimerize or associate with another effector protein in a multimeric complex. Multimeric complex formation is further described herein.

### Nucleic Acid Modification Activity

In some embodiments, fusion partners have enzymatic activity that modifies a nucleic acid, such as a target nucleic acid. In some embodiments, the target nucleic acid may comprise or consist of a ssRNA, dsRNA, ssDNA, or a dsDNA. Examples of enzymatic activity that modifies the target nucleic acid include, but are not limited to: nuclease activity, which comprises the enzymatic activity of an enzyme which allows the enzyme to cleave the phosphodiester bonds between the nucleotide subunits of nucleic acids, such as that provided by a restriction enzyme, or a nuclease (e.g., FokI nuclease); methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y, human immunodeficiency virus type 1 integrase (IN), Tn3 resolvase); transposase activity; recombinase activity such as that provided by a recombinase (e.g., catalytic domain of Gin recombinase); polymerase activity; ligase activity; helicase activity; photolyase activity; and glycosylase activity.

In some embodiments, fusion effector proteins modify a target nucleic acid or the expression thereof, wherein the target nucleic acid comprises a deoxyribonucleoside, a ribonucleoside or a combination thereof. The target nucleic acid may comprise or consist of a single stranded RNA (ssRNA), a double-stranded RNA (dsRNA), a single-stranded DNA (ssDNA), or a double stranded DNA (dsDNA). In some embodiments, fusion partners target a ssRNA, dsRNA, ssDNA, or a dsDNA. In some embodiments, fusion partners target ssRNA. Non-limiting examples of fusion partners for modifying ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins.

It is understood that a fusion partner may include an entire protein, or in some embodiments, may include a fragment of the protein (e.g., a functional domain). In some embodiments, the functional domain binds or interacts with a nucleic acid, such as ssRNA, including intramolecular and/or intermolecular secondary structures thereof (e.g., hairpins, stem-loops, etc.). The functional domain may interact transiently or irreversibly, directly, or indirectly. In some embodiments, a functional domain comprises a region of one or more amino acids in a protein that is required for an activity of the protein, or the full extent of that activity, as measured in an in vitro assay. Activities include but are not limited to nucleic acid binding, nucleic acid editing, nucleic acid mutating, nucleic acid modifying, nucleic acid cleaving, protein binding or combinations thereof. The absence of the functional domain, including mutations of the functional domain, would abolish or reduce activity.

Accordingly, fusion partners may comprise a protein or domain thereof selected from: endonucleases (e.g., RNase III, the CRR22 DYW domain, Dicer, and PIN (PilT N-terminus); SMG5 and SMG6; domains responsible for stimulating RNA cleavage (e.g., CPSF, CstF, CFIm and CFIIm); exonucleases such as XRN-1 or Exonuclease T; deadenylases such as HNT3; protein domains responsible for nonsense mediated RNA decay (e.g., UPF1, UPF2, UPF3, UPF3b, RNP S1, Y14, DEK, REF2, and SRm160); protein domains responsible for stabilizing RNA (e.g., PABP); proteins and protein domains responsible for polyadenylation of RNA (e.g., PAP1, GLD-2, and Star-PAP); proteins and protein domains responsible for polyuridinylation of RNA (e.g., CI D1 and terminal uridylate transferase); and other suitable domains that affect nucleic acid modifications.

In some embodiments, an effector protein is a fusion protein, wherein the effector protein is fused to a chromatin-modifying enzyme. In some embodiments, the fusion protein chemically modifies a target nucleic acid, for example by methylating, demethylating, or acetylating the target nucleic acid in a sequence specific or non-specific manner.

### Base Editors

In some embodiments, fusion partners edit a nucleobase of a target nucleic acid. Fusion proteins comprising such a fusion partner and an effector protein may be referred to as base editors, wherein the fusion partner is a base editing enzyme. A base editor can refer to a fusion protein comprising a base editing enzyme fused to an effector protein. Fusion proteins comprising such fusion partners and a catalytically inactive Cas effector protein may be referred to as base editors. The base editor is functional when the effector protein is coupled to a guide nucleic acid. In some embodiments, the base editor is functional when the effector protein is coupled to a target nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

A base editing enzyme can refer to a protein, polypeptide or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). It is understood that any reference to a base editing enzyme herein also refers to a base editing enzyme variant.

In some embodiments, fusion partners modify a nucleobase of a target nucleic acid. Non-limiting examples of the type of modification that a base editing enzyme is capable of catalyzing includes converting an existing nucleobase to a different nucleobase, such as: converting a cytosine to a guanine or thymine or converting an adenine to a guanine, hydrolytic deamination of an adenine or adenosine; or methylation of cytosine (e.g., CpG, CpA, CpT or CpC). In some embodiments, base editors edit a nucleobase on a ssDNA. In some embodiments, base editors edit a nucleobase on both strands of dsDNA. In some embodiments, base editors edit a nucleobase of an RNA.

In some embodiments, base editors modify a sequence of a target nucleic acid. In some embodiments, base editors provide a nucleobase change in a DNA molecule. In some embodiments, the nucleobase change in the DNA molecule is selected from: an adenine (A) to guanine (G); cytosine (C) to thymine (T); and cytosine (C) to guanine (G). In some embodiments, base editors provide a nucleobase change in an RNA molecule. In some embodiments, the nucleobase change in the RNA molecule is selected from: adenine (A) to guanine (G); uracil (U) to cytosine (C); cytosine (C) to guanine (G); and guanine (G) to adenine (A).

A base editing enzyme itself may or may not bind to the nucleic acid molecule containing the nucleobase. In some embodiments, upon binding to its target locus in the target nucleic acid (e.g., a DNA molecule), base pairing between the guide nucleic acid and target strand leads to displacement of a small segment of ssDNA in an “R-loop”. In some embodiments, DNA bases within the R-loop are edited by the base editor having the deaminase enzyme activity. In some embodiments, base editors for improved efficiency in eukaryotic cells comprise a catalytically inactive effector protein that may generate a nick in the non-edited strand, inducing repair of the non-edited strand using the edited strand as a template.

In some embodiments, a base editing enzyme comprises a deaminase enzyme. Exemplary deaminases are described in US20210198330, WO2021041945, WO2021050571A1, and WO2020123887, all of which are incorporated herein by reference in their entirety. Exemplary deaminase domains are described WO 2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3: eaao4774 (2017), and Rees et al., Nat Rev Genet. 2018 December; 19 (12): 770-788. doi: 10.1038/s41576-018-0059-1, which are hereby incorporated by reference in their entirety. In some embodiments, the deaminase functions as a monomer. In some embodiments, the deaminase functions as heterodimer with an additional protein. In some embodiments, base editors comprise a DNA glycosylase inhibitor (e.g., an uracil glycosylase inhibitor (UGI) or uracil N-glycosylase (UNG)). In some embodiments, the fusion partner is a deaminase, e.g., ADAR1/2, ADAR-2, AID, or any functional variant thereof.

In some embodiments, a base editor is a cytosine base editor (CBE). In some embodiments, the CBE may convert a cytosine to a thymine. In some embodiments, a cytosine base editing enzyme may accept ssDNA as a substrate but may not be capable of cleaving dsDNA, as fused to a catalytically inactive effector protein. In some embodiments, when bound to its cognate DNA, the catalytically inactive effector protein of the CBE may perform local denaturation of the DNA duplex to generate an R-loop in which the DNA strand not paired with a guide nucleic acid exists as a disordered single-stranded bubble. In some embodiments, the catalytically inactive effector protein generated ssDNA R-loop may enable the CBE to perform efficient and localized cytosine deamination in vitro. In some embodiments, deamination activity is exhibited in a window of about 4 to about 10 base pairs. In some embodiments, fusion to the catalytically inactive effector protein presents a target site to the cytosine base editing enzyme in high effective molarity, which may enable the CBE to deaminate cytosines located in a variety of different sequence motifs, with differing efficacies. In some embodiments, the CBE is capable of mediating RNA-programmed deamination of target cytosines in vitro or in vivo. In some embodiments, the cytosine base editing enzyme is a cytidine deaminase. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C·G-to-G·C base editors developed using CRISPRi screens, target-library analysis, and machine learning,” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments, CBEs comprise a uracil glycosylase inhibitor (UGI) or uracil N-glycosylase (UNG). In some embodiments, base excision repair (BER) of U·G in DNA is initiated by a UNG, which recognizes a U·G mismatch and cleaves the glyosidic bond between a uracil and a deoxyribose backbone of DNA. In some embodiments, BER results in the reversion of the U·G intermediate created by the first CBE back to a C·G base pair. In some embodiments, the UNG may be inhibited by fusion of a UGI. In some embodiments, the CBE comprises a UGI. In some embodiments, a C-terminus of the CBE comprises the UGI. In some embodiments, the UGI is a small protein from bacteriophage PBS. In some embodiments, the UGI is a DNA mimic that potently inhibits both human and bacterial UNG. In some embodiments, the UGI inhibitor is any protein or polypeptide that inhibits UNG. In some embodiments, the CBE may mediate efficient base editing in bacterial cells and moderately efficient editing in mammalian cells, enabling conversion of a C·G base pair to a T·A base pair through a U·G intermediate. In some embodiments, the CBE is modified to increase base editing efficiency while editing more than one strand of DNA.

In some embodiments, a CBE nicks a non-edited DNA strand. In some embodiments, the non-edited DNA strand nicked by the CBE biases cellular repair of a U·G mismatch to favor a U·A outcome, elevating base editing efficiency. In some embodiments, a APOBEC1-nickase-UGI fusion efficiently edits in mammalian cells, while minimizing frequency of non-target indels. In some embodiments, base editors do not comprise a functional fragment of the base editing enzyme. In some embodiments, base editors do not comprise a function fragment of a UGI, where such a fragment may be capable of excising a uracil residue from DNA by cleaving an N-glycosidic bond.

In some embodiments, the fusion protein further comprises a non-protein uracil-DNA glycosylase inhibitor (npUGI). In some embodiments, the npUGI is selected from a group of small molecule inhibitors of uracil-DNA glycosylase (UDG), or a nucleic acid inhibitor of UDG. In some embodiments, the npUGI is a small molecule derived from uracil. Examples of small molecule non-protein uracil-DNA glycosylase inhibitors, fusion proteins, and Cas-CRISPR systems comprising base editing activity are described in WO2021087246, which is incorporated by reference in its entirety.

In some embodiments, a cytosine base editing enzyme, and therefore a cytosine base editor, is a cytidine deaminase. In some embodiments, the cytidine deaminase base editor is generated by ancestral sequence reconstruction as described in WO2019226953, which is hereby incorporated by reference in its entirety. Non-limiting exemplary cytidine deaminases suitable for use with effector proteins described herein include: APOBEC1, APOBEC2, APOBEC3C, APOBEC3D, APOBEC3G, APOBEC3H, APOBEC4, APOBEC3A, BE1 (APOBEC1-XTEN-dCas9), BE2 (APOBEC1-XTEN-dCas9-UGI), BE3 (APOBEC1-XTEN-dCas9 (A840H)-UGI), BE3-Gam, saBE3, saBE4-Gam, BE4, BE4-Gam, saBE4, and saBE4-Gam as described in WO2021163587, WO2021087246, WO2021062227, and WO2020123887, which are incorporated herein by reference in their entirety.

In some embodiments, a base editor is a cytosine to guanine base editor (CGBE). A CGBE may convert a cytosine to a guanine.

In some embodiments, a base editor is an adenine base editor (ABE). An ABE may convert an adenine to a guanine. In some embodiments, an ABE converts an A·T base pair to a G·C base pair. In some embodiments, the ABE converts a target A·T base pair to G·C in vivo or in vitro. In some embodiments, ABEs provided herein reverse spontaneous cytosine deamination, which has been linked to pathogenic point mutations. In some embodiments, ABEs provided herein enable correction of pathogenic SNPs (˜47% of disease-associated point mutations). In some embodiments, the adenine comprises exocyclic amine that has been deaminated (e.g., resulting in altering its base pairing preferences). In some embodiments, deamination of adenosine yields inosine. In some embodiments, inosine exhibits the base-pairing preference of guanine in the context of a polymerase active site, although inosine in the third position of a tRNA anticodon is capable of pairing with A, U, or C in mRNA during translation. Non-limiting exemplary adenine base editing enzymes suitable for use with effector proteins described herein include: ABE8e, ABE8.20m, APOBEC3A, Anc APOBEC (a.k.a. AncBE4Max), and BtAPOBEC2. Non-limiting exemplary ABEs suitable for use herein include: ABE7, ABE8.1m, ABE8.2m, ABE8.3m, ABE8.4m, ABE8.5m, ABE8.6m, ABE8.7m, ABE8.8m, ABE8.9m, ABE8.10m, ABE8.11m, ABE8.12m, ABE8.13m, ABE8.14m, ABE8.15m, ABE8.16m, ABE8.17m, ABE8.18m, ABE8.19m, ABE8.20m, ABE8.21m, ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d, and ABE8.24d. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871.

In some embodiments, an adenine base editing enzyme of an ABE is an adenosine deaminase. Non-limiting exemplary adenosine base editors suitable for use herein include ABE9. In some embodiments, the ABE comprises an engineered adenosine deaminase enzyme capable of acting on ssDNA. The engineered adenosine deaminase enzyme may be an adenosine deaminase variant that differs from a naturally occurring deaminase. Relative to the naturally occurring deaminase, the adenosine deaminase variant may comprise one or more amino acid alteration, including a V82S alteration, a T166R alteration, a Y147T alteration, a Y147R alteration, a Q154S alteration, a Y123H alteration, a Q154R alteration, or a combination thereof.

In some embodiments, a base editor comprises a deaminase dimer. In some embodiments, the base editor further comprising a base editing enzyme and an adenine deaminase (e.g., TadA). In some embodiments, the adenosine deaminase is a TadA monomer (e.g., Tad*7.10, TadA*8 or TadA*9). In some embodiments, the adenosine deaminase is a TadA*8 variant (e.g., any one of TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entirety). In some embodiments, the base editor comprises a base editing enzyme fused to TadA by a linker (e.g., wherein the base editing enzyme is fused to TadA at N-terminus or C-terminus by a linker).

In some embodiments, TadA comprises or consists of at least a portion of the sequence:

(SEQ ID NO: 143)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRM
RRQEIKAQKKAQSSTD.

In some embodiments, a base editing enzyme is a deaminase dimer comprising an ABE. In some embodiments, the deaminase dimer comprises an adenosine deaminase. In some embodiments, the deaminase dimer comprises TadA fused to a suitable adenine base editing enzyme including an: ABE8e, ABE8.20m, APOBEC3A, Anc APOBEC (a.k.a. AncBE4Max), BtAPOBEC2, and variants thereof. In some embodiments, the adenine base editing enzyme is fused to amino-terminus or the carboxy-terminus of TadA.

Some base editors modify a nucleobase of an RNA. In some embodiments, RNA base editors comprise an adenosine deaminase. In some embodiments, ADAR proteins bind to RNAs and alter their sequence by changing an adenosine into an inosine. In some embodiments, RNA base editors comprise a Cas effector protein that is activated by or binds RNA.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions, devices, kits, methods and systems described herein comprise a base editor and a guide nucleic acid, wherein the guide nucleic acid directs the base editor to a sequence in a target gene. The target gene may be associated with a disease. In some embodiments, the guide nucleic acid directs that base editor to or near a mutation in the sequence of a target gene. The mutation may be the deletion of one or more nucleotides. The mutation may be the addition of one or more nucleotides. The mutation may be the substitution of one or more nucleotides. The mutation may be the insertion, deletion, or substitution of a single nucleotide, also referred to as a point mutation. The point mutation may be a SNP. The mutation may be associated with a disease. In some embodiments, the guide nucleic acid directs the base editor to bind a target sequence within the target nucleic acid that is within 0, 1, 2, 3, 4, 5, 6, 7, 8, 9, 10, 11, 12, 13, 14, 15, 16, 17, 18, 19, or 20 nucleotides of the mutation. In some embodiments, the guide nucleic acid comprises a sequence that is identical, complementary, or reverse complementary to a target sequence of a target nucleic acid that comprises the mutation. In some embodiments, the guide nucleic acid comprises a sequence that is identical, complementary, or reverse complementary to a target sequence of a target nucleic acid that is within 1, 2, 3, 4, 5, 6, 7, 8, 9, 10, 11, 12, 13, 14, 15, 16, 17, 18, 19, or 20 nucleotides of the mutation.

### Prime Editing

In some embodiments, a fusion protein and/or a fusion partner can comprise a prime editing enzyme. In some embodiments, a prime editing enzyme comprises a reverse transcriptase. A non-limiting example of a reverse transcriptase is an M-MLV RT enzyme and variants thereof having polymerase activity. In some embodiments, the M-MLV RT enzyme comprises at least one mutation selected from D200N, L603W, T330P, T306K, and W313F relative to wildtype M-MLV RT enzyme.

In some embodiments, a prime editing enzyme may require a prime editing guide RNA (pegRNA) to catalyze an editing. Such a pegRNA may be capable of identifying a target nucleotide or target sequence in a target nucleic acid to be edited and encoding a new genetic information that replaces the target nucleotide or target sequence in the target nucleic acid. A prime editing enzyme may require a pegRNA and a single guide RNA to catalyze the editing. In some embodiments, the target nucleic acid is a dsDNA molecule. In some embodiments, the pegRNA comprises a guide RNA comprising a first region that is bound by the effector protein, and a second region comprising a spacer sequence that is complementary to a target sequence of the dsDNA molecule; a template RNA comprising a primer binding sequence that hybridizes to a primer sequence of the dsDNA molecule that is formed when target nucleic acid is cleaved, and a template sequence that is complementary to at least a portion of the target sequence of the dsDNA molecule with the exception of at least one nucleotide. In some embodiments, the spacer sequence is complementary to the target sequence on a target strand of the dsDNA molecule. In some embodiments, the spacer sequence is complementary to the target sequence on a non-target strand of the dsDNA molecule. In some embodiments, the primer binding sequence hybridizes to a primer sequence on the non-target strand of the dsDNA molecule. In some embodiments, the primer binding sequence hybridizes to a primer sequence on the target strand of the dsDNA molecule. In some embodiments, the target strand is cleaved. In some embodiments, the non-target strand is cleaved.

### Protein Modification Activity

In some embodiments, a fusion partner provides enzymatic activity that modifies a protein associated with a target nucleic acid. The protein may be a histone, an RNA binding protein, or a DNA binding protein. Examples of such protein modification activities include: methyltransferase activity, such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity; phosphatase activity; ubiquitin ligase activity; deubiquitinating activity; adenylation activity; deadenylation activity; SUMOylating activity; deSUMOylating activity; ribosylation activity; deribosylation activity; myristoylation activity; and demyristoylation activity.

### CRISPRa Fusions and CRISPRi Fusions

In some embodiments, fusion partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some embodiments, fusion partners that increase or decrease transcription include a transcription activator domain or a transcription repressor domain, respectively.

In some embodiments, fusion partners activate or increase expression of a target nucleic acid. Such fusion proteins comprising the described fusion partners and an effector protein may be referred to as CRISPRa fusions. In some embodiments, fusion partners increase expression of the target nucleic acid relative to its expression in the absence of the fusion effector protein. Relative expression, including transcription and RNA levels, may be assessed, quantified, and compared, e.g., by RT-qPCR. In some embodiments, fusion partners comprise a transcriptional activator. Transcriptional activators may promote transcription via: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof. In some embodiments, the fusion partner is a reverse transcriptase.

Non-limiting examples of fusion partners that promote or increase transcription include: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2, and ROS1; and functional domains thereof. Other non-limiting examples of suitable fusion partners include: proteins and protein domains responsible for stimulating translation (e.g., Staufen); proteins and protein domains responsible for (e.g., capable of) modulating translation (e.g., translation factors such as initiation factors, elongation factors, release factors, etc., e.g., eIF4G); proteins and protein domains responsible for stimulation of RNA splicing (e.g., Serine/Arginine-rich (SR) domains); and proteins and protein domains responsible for stimulating transcription (e.g., CDK7 and HIV Tat).

In some embodiments, fusion partners inhibit or reduce expression of a target nucleic acid. Such fusion proteins comprising described fusion partners and an effector protein may be referred to as CRISPRi fusions. In some embodiments, fusion partners reduce expression of the target nucleic acid relative to its expression in the absence of the fusion effector protein. Relative expression, including transcription and RNA levels, may be assessed, quantified, and compared, e.g., by RT-qPCR. In some embodiments, fusion partners may comprise a transcriptional repressor. Transcriptional repressors may inhibit transcription via: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof.

Non-limiting examples of fusion partners that decrease or inhibit transcription include, but are not limited to: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY; histone lysine deacetylases such as HDAC1, HDAC2, HDAC3, HDAC8, HDAC4, HDAC5, HDAC7, HDAC9, SIRT1, SIRT2, HDAC11; and DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A, and Lamin B; and functional domains thereof. Other non-limiting examples of suitable fusion partners include: proteins and protein domains responsible for repressing translation (e.g., Ago2 and Ago4); proteins and protein domains responsible for repression of RNA splicing (e.g., PTB, Sam68, and hnRNP A1); proteins and protein domains responsible for reducing the efficiency of transcription (e.g., FUS (TLS)).

In some embodiments, fusion proteins are targeted by a guide nucleic acid (e.g., guide RNA) to a specific location in a target nucleic acid and exert locus-specific regulation such as blocking RNA polymerase binding to a promoter (which selectively inhibits transcription activator function), and/or changes a local chromatin status (e.g., when a fusion sequence is used that edits the target nucleic acid or modifies a protein associated with the target nucleic acid). In some embodiments, the modifications are transient (e.g., transcription repression or activation). In some embodiments, the modifications are inheritable. For example, epigenetic modifications made to a target nucleic acid, or to proteins associated with the target nucleic acid, e.g., nucleosomal histones, in a cell, can be observed in a successive generation.

In some embodiments, fusion partners comprise an RNA splicing factor. The RNA splicing factor may be used (in whole or as fragments thereof) for modular organization, with separate sequence-specific RNA binding modules and splicing effector domains. Non-limiting examples of RNA splicing factors include members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP Al binds to exonic splicing silencers (ESSs) through its RRM domains and inhibits exon inclusion through a C-terminal Glycine-rich domain. Some splicing factors may regulate alternative use of splice site (ss) by binding to regulatory sequences between the two alternative sites. For example, ASF/SF2 may recognize ESEs and promote the use of intron proximal sites, whereas hnRNP Al may bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple c\acute{ω}-elements that are located in either the core exon region or the exon extension region (i.e., between the two alternative 5′ splice sites). For more examples, see WO 2010/075303, which is hereby incorporated by reference in its entirety.

### Recombinases

In some embodiments, fusion partners comprise a recombinase. In some embodiments, effector proteins described herein are fused with the recombinase. In some embodiments, the effector proteins have reduced nuclease activity or no nuclease activity. In some embodiments, the recombinase is a site-specific recombinase.

In some embodiments, a catalytically inactive effector protein is fused with a recombinase, wherein the recombinase can be a site-specific recombinase. Such polypeptides can be used for site-directed transgene insertion. Non-limiting examples of site-specific recombinases include a tyrosine recombinase (e.g., Cre, Flp or lambda integrase), a serine recombinase (e.g., gamma-delta resolvase, Tn3 resolvase, Sin resolvase, Gin invertase, Hin invertase, Tn5044 resolvase, IS607 transposase and integrase), or mutants or variants thereof. In some embodiments, the recombinase is a serine recombinase. Non-limiting examples of serine recombinases include gamma-delta resolvase, Tn3 resolvase, Sin resolvase, Gin invertase, Hin invertase, Tn5044 resolvase, IS607 transposase, and IS607 integrase. In some embodiments, the site-specific recombinase is an integrase. Non-limiting examples of integrases include: Bxb1, wBeta, BL3, phiR4, A118, TG1, MR11, phi370, SPBc, TP901-1, phiRV, FC1, K38, phiBT1, and phiC31. Further discussion and examples of suitable recombinase fusion partners are described in U.S. Pat. No. 10,975,392, which is incorporated herein by reference in its entirety. In some embodiments, the fusion protein comprises a linker that links the recombinase to the Cas-CRISPR domain of the effector protein. In some embodiments, the linker is The-Ser.

### Linkers for Peptides

In some embodiments, a linker comprises a bond or molecule that links a first polypeptide to a second polypeptide. In general, effector proteins and fusion partners of a fusion effector protein are connected via a linker. The linker may comprise or consist of a covalent bond. The linker may comprise or consist of a chemical group. In some embodiments, the linker comprises an amino acid. In some embodiments, a peptide linker comprises at least two amino acids linked by an amide bond. In some embodiments, the effector protein and the fusion partner are directly linked by a covalent bond.

In general, the linker connects a terminus of the effector protein to a terminus of the fusion partner. In some embodiments, the carboxy terminus of the effector protein is linked to the amino terminus of the fusion partner. In some embodiments, the carboxy terminus of the fusion partner is linked to the amino terminus of the effector protein.

In some embodiments, linkers comprise one or more amino acids. In some embodiments, linker is a protein. In some embodiments, a terminus of the effector protein is linked to a terminus of the fusion partner through an amide bond. In some embodiments, a terminus of the effector protein is linked to a terminus of the fusion partner through a peptide bond. In some embodiments, linkers comprise an amino acid. In some embodiments, linkers comprise a peptide. In some embodiments, an effector protein is coupled to a fusion partner by a linker protein. In some embodiments, the linker may have any of a variety of amino acid sequences. In some embodiments, fusion effector proteins disclosed herein comprise a linker, wherein the linker comprises or consists of a peptide. The peptide may comprise a region of rigidity (e.g., beta sheet, alpha helix), a region of flexibility, or any combination thereof. In some embodiments, the linker comprises small amino acids, such as glycine and alanine, that impart linker flexibility. In some embodiments, the linker comprises amino acids that impart linker rigidity, such as valine and isoleucine. The ordinarily skilled artisan will recognize that design of a peptide conjugated to any desired element may include linkers that are all or partially flexible, such that the linker may include a flexible linker as well as one or more portions that confer less flexible structure. Suitable linkers include proteins of 4 linked amino acids to 40 linked amino acids in length, or between 4 linked amino acids and 25 linked amino acids in length. In some embodiments, linked amino acids described herein comprise at least two amino acids linked by an amide bond.

These linkers may be produced by using synthetic, linker-encoding oligonucleotides to couple the proteins, or may be encoded by a nucleic acid sequence encoding a fusion effector protein (e.g., an effector protein coupled to a fusion partner). Linkers may comprise glycine(s), serine(s), and combinations thereof. In some embodiments, the linker is from 1 to 100 amino acids in length. In some embodiments, the linker is more 100 amino acids in length. In some embodiments, the linker is from 10 to 27 amino acids in length. In some embodiments, linker proteins include glycine polymers (G)n, glycine-serine polymers (including, for example, (GS)n, GSGGSn, GGSGGSn, and GGGSn, where n is an integer of at least one), glycine-alanine polymers, and alanine-serine polymers. In some embodiments, linkers may comprise amino acid sequences including, but not limited to, GGSG, GGSGG, GSGSG, GSGGG, GGGSG, and GSSSG. In some embodiments, the linker comprises one or more repeats a tri-peptide GGS. In some embodiments, the linker is an XTEN linker. In some embodiments, the XTEN linker is an XTEN80 linker. In some embodiments, the XTEN linker is an XTEN20 linker. In some embodiments, the XTEN20 linker has an amino acid sequence of GSGGSPAGSPTSTEEGTSESATPGSG (SEQ ID NO: 171). In some embodiments the linker comprises or consists of at least a portion of the sequence: SGGSSGGSSGSETPGTSESATPESSGGSSGGS (SEQ ID NO: 144). In some embodiments, the amino acid sequence of the linker is 70%, 75%, 80%, 85%, 90%, or 95% identical to SGGSSGGSSGSETPGTSESATPESSGGSSGGS (SEQ ID NO: 144).

In some embodiments, linkers do not comprise an amino acid. In some embodiments, linkers do not comprise a peptide. In some embodiments, linkers comprise or consist of a non-peptide linker. In some embodiments, linkers comprise a nucleotide, a polynucleotide, a polymer, or a lipid. Non-limiting examples of non-peptide linkers are linkers comprising polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, an alkyl linker, or a combination thereof.

In some embodiments, linkers comprise or consist of a nucleic acid. In some embodiments, the nucleic acid comprises DNA. In some embodiments, the nucleic acid comprises RNA. In some embodiments, the effector protein and the fusion partner each interact with the nucleic acid, the nucleic acid thereby linking the effector protein and the fusion partner. In some embodiments, the nucleic acid serves as a scaffold for both the effector protein and the fusion partner to interact with, thereby linking the effector protein and the fusion partner. Such nucleic acids include those described by Tadakuma et al., (2016), Progress in Molecular Biology and Translational Science, Volume 139, 2016, Pages 121-163, incorporated herein by reference.

In some embodiments, the fusion effector protein or the guide nucleic acid comprises a chemical modification that allows for direct crosslinking between the guide nucleic acid or the effector protein and the fusion partner. By way of non-limiting example, the chemical modification may comprise any one of a SNAP-tag, CLIP-tag, ACP-tag, Halo-tag, and an MCP-tag. In some embodiments, modifications are introduced with a Click Reaction, also known as Click Chemistry. The Click reaction may be copper dependent or copper independent.

In some embodiments, guide nucleic acids comprise an aptamer. The aptamer may serve as a linker between the effector protein and the fusion partner by interacting non-covalently with both. In some embodiments, the aptamer binds a fusion partner, wherein the fusion partner is a transcriptional activator. In some embodiments, the aptamer binds a fusion partner, wherein the fusion partner is a transcriptional inhibitor. In some embodiments, the aptamer binds a fusion partner, wherein the fusion partner comprises a base editor. In some embodiments, the aptamer binds the fusion partner directly. In some embodiments, the aptamer binds the fusion partner indirectly. Aptamers may bind the fusion partner indirectly through an aptamer binding protein. By way of non-limiting example, the aptamer binding protein may be MS2 and the aptamer sequence may be ACATGAGGATCACCCATGT (SEQ ID NO: 36); the aptamer binding protein may be PP7 and the aptamer sequence may be GGAGCAGACGATATGGCGTCGCTCC (SEQ ID NO: 37); or the aptamer binding protein may be BoxB and the aptamer sequence may be GCCCTGAAGAAGGGC (SEQ ID NO: 38).

In some embodiments, the fusion partner is located within effector protein. For example, the fusion partner may be a domain of a fusion partner protein that is internally integrated into the effector protein. In other words, the fusion partner may be located between the 5′ and 3′ ends of the effector protein without disrupting the ability of the fusion effector protein to recognize/bind a target nucleic acid. In some embodiments, the fusion partner replaces a portion of the effector protein. In some embodiments, the fusion partner replaces a domain of the effector protein. In some embodiments, the fusion partner does not replace a portion of the effector protein.

### Effector Protein Activity

Engineered proteins (i.e., effector proteins) of the present disclosure may provide an increased or enhanced activity relative to a naturally occurring protein. Engineered proteins (i.e., effector proteins) of the present disclosure may provide a reduced activity relative to a naturally occurring protein. Engineered proteins of the present disclosure may show an enhanced activity or reduced activity, when measured in a cleavage assay or a reporter assay, under certain conditions relative to a control condition. The activity may be nuclease activity. The activity may be nickase activity. The activity may be nucleic acid binding activity. Engineered proteins may provide an increased or reduced activity relative to a naturally occurring protein under a given condition of a cell or sample in which the activity occurs. For example, the effector proteins of the present disclosure may have variable levels of activity based on conditions such as buffer formulation, pH level, temperature, or salt. Buffers consistent with the present disclosure include phosphate buffers, Tris buffers, and HEPES buffers.

In some embodiments, effector proteins of the present disclosure exhibit enhanced or increased activity at under certain conditions relative to a control condition. For example, the condition may be temperature. In some embodiments, the temperature may be at least about 25° C., at least about 30° C., at least about 35° C., at least 37° C., at least about 40° C., at least about 50° C., at least about 65° C., at least about 70° C., at least about 75° C. In some embodiments, the temperature is not greater than 80° C. In some embodiments, the temperature is about 25° C., about 30° C., about 35° C., about 37° C., about 40° C., about 45° C., about 50° C., about 55° C., about 60° C., about 65° C., about 70° C., about 75° C., about 80° C., about 85° C. or about 90° C. In some embodiments, the temperature is about 25° C. to about 45° C., about 35° C. to about 55° C., about 37° C. to about 60° C. or about 55° C. to about 65° C. In some embodiments, the temperature is about 37° C. to about 45° C., about 37° C. to about 50° C., about 37° C. to about 55° C., about 37° C. to about 60° C., or about 37° C. to about 65° C.

As another example, the condition may be the presence of one or more salt, including a combination of salts. Accordingly, in some embodiments, the salt may be one or more salt selected from a magnesium salt, a zinc salt, a potassium salt, a calcium salt, and a sodium salt. In some embodiments, the salt is a combination of two or more salts. For example, in some embodiments, the salt is a combination of two or more salts selected from a magnesium salt, a zinc salt, a potassium salt, a calcium salt and a sodium salt. In some embodiments, the salt is magnesium acetate. In some embodiments, the salt is magnesium chloride. In some embodiments, the salt is potassium acetate. In some embodiments, the salt is potassium nitrate. In some embodiments, the salt is zinc chloride. In embodiments, the salt is sodium chloride. In some embodiments, the salt is potassium chloride.

As yet another example, the condition may be the concentration of the one or more salt. Accordingly, in some embodiments, the concentration of the salt can be about 0.001 mM to about 500 mM. In some embodiments, the concentration of the salt is about 0.001 mM to about 400 mM. In some embodiments, the concentration of the salt is about 0.001 mM to about 300 mM. In some embodiments, the concentration of the salt is about 0.001 mM to about 200 mM. In some embodiments, the concentration of the salt is about 0.001 mM to about 100 mM. In some embodiments, the concentration of the salt is about 0.001 mM to about 10 mM. In some embodiments, the concentration of the salt is about 0.01 mM to about 500 mM. In some embodiments, the concentration of the salt is about 0.01 mM to about 400 mM. In some embodiments, the concentration of the salt is about 0.01 mM to about 300 mM. In some embodiments, the concentration of the salt is about 0.01 mM to about 200 mM. In some embodiments, the concentration of the salt is about 0.01 mM to about 100 mM. In some embodiments, the concentration of the salt is about 0.01 mM to about 10 mM. In some embodiments, the concentration of the salt is about 0.1 mM to about 500 mM. In some embodiments, the concentration of the salt is about 0.1 mM to about 400 mM. In some embodiments, the concentration of the salt is about 0.1 mM to about 300 mM. In some embodiments, the concentration of the salt is about 0.1 mM to about 200 mM. In some embodiments, the concentration of the salt is about 0.1 mM to about 100 mM. In some embodiments, the concentration of the salt is about 0.1 mM to about 10 mM. In some embodiments, the concentration of the salt is about 1 mM to about 500 mM. In some embodiments, the concentration of the salt is about 1 mM to about 400 mM. In some embodiments, the concentration of the salt is about 1 mM to about 300 mM. In some embodiments, the concentration of the salt is about 1 mM to about 200 mM. In some embodiments, the concentration of the salt is about 1 mM to about 100 mM. In some embodiments, the concentration of the salt is about 1 mM to about 10 mM. In some embodiments, the concentration of the salt is about 10 mM to about 500 mM. In some embodiments, the concentration of the salt is about 10 mM to about 400 mM. In some embodiments, the concentration of the salt is about 10 mM to about 300 mM. In some embodiments, the concentration of the salt is about 10 mM to about 200 mM. In some embodiments, the concentration of the salt is about 10 mM to about 100 mM. In some embodiments, the concentration of the salt is about 100 mM to about 500 mM. In some embodiments, the concentration of the salt is about 100 mM to about 400 mM. In some embodiments, the concentration of the salt is about 100 mM to about 300 mM. In some embodiments, the concentration of the salt is about 100 mM to about 200 mM.

In some embodiments, the salt is potassium acetate and the concentration of salt in the solution is about 100 mM. In some embodiments, the salt is potassium acetate or sodium chloride and the concentration of salt in the solution is about 200 mM. In some embodiments, the salt is potassium acetate or sodium chloride and the concentration of salt in the solution is about 100 mM to about 200 mM.

In another example, the condition may be in the presence of PH levels between about pH 7 to about pH 9. Accordingly, in some embodiments, the condition is the presence of pH level at about pH 7, about pH 7.1, about pH 7.2, about pH 7.3, about pH 7.4, about pH 7.5, about pH 7.6, about pH 7.7, about pH 7.8, about pH 7.9, about pH 8, about pH 8.1, about pH 8.2, about pH 8.3, about pH 8.4, about pH 8.5, about pH 8.6, about pH 8.7, about pH 8.8, about pH 8.9, about pH 9, from pH 7 to 7.5, from pH 7.5 to 8, from pH 8 to 8.5, from pH 8.5 to 9, or from pH 7 to 8.5.

In some embodiments, effector proteins of the present disclosure may exhibit activity or enhanced activity in a solution at a room temperature viscosity of less than about 15 centipoise, less than about 12 centipoise, less than about 10 centipoise, less than about 8 centipoise, less than about 6 centipoise, less than about 5 centipoise, less than about 4 centipoise, less than about 3 centipoise, less than about 2 centipoise, or less than about 1.5 centipoise.

In some embodiments, effector proteins of the present disclosure may exhibit activity or enhanced activity in a solution comprising an ionic strength of less than about 500 mM, less than about 400 mM, less than about 300 mM, less than about 250 mM, less than about 200 mM, less than about 150 mM, less than about 100 mM, less than about 80 mM, less than about 60 mM, or less than about 50 mM. In some embodiments, effector proteins may exhibit activity or enhanced activity with an assay excipient, which may stabilize a reagent or product, prevent aggregation or precipitation, or enhance or stabilize a detectable signal (e.g., a fluorescent signal). Examples of assay excipients include, but are not limited to, saccharides and saccharide derivatives (e.g., sodium carboxymethyl cellulose and cellulose acetate), detergents, glycols, polyols, esters, buffering agents, alginic acid, and organic solvents (e.g., DMSO).

In some embodiments, effector proteins of the present disclosure may exhibit activity or enhanced activity in the presence of a co-factor. In some embodiments, the co-factor allows the effector proteins to perform a function. In some embodiments, the function is pre-crRNA processing and/or target nucleic acid cleavage. As discussed in Jiang F. and Doudna J. A. (Annu. Rev. Biophys. 2017. 46:505-29), Cas9 uses divalent metal ions as co-factors. The suitability of a divalent metal ion as a cofactor can easily be assessed, such as by methods based on those described by Sundaresan et al. (Cell Rep. 2017 Dec. 26; 21 (13): 3728-3739). In some embodiments, the co-factor is a divalent metal ion. Non-limiting exemplary divalent metal ions include: Mg²⁺, Mn²⁺, Zn²⁺, Ca²⁺, and Cu²⁺. In some embodiments, the effector protein forms a complex with a divalent metal ion. In some embodiments, the effector protein forms a complex with Mg²⁺, Mn²⁺, Zn²⁺, Ca²⁺, or Cu².

### Thermostable Effector Proteins

In some embodiments, an effector protein may be thermostable. In some embodiments, a thermostable effector protein may have an enhanced activity as described herein. In some embodiments, known effector proteins (e.g., Cas12 nucleases) are relatively thermo-sensitive and only exhibit activity (e.g., cis and/or trans cleavage) sufficient to produce a detectable signal in a diagnostic assay at temperatures less than 40° C., and optimally at about 37° C. A thermostable protein may have enzymatic activity, stability, or folding comparable to those at 37° C. In some embodiments, the trans cleavage activity (e.g., the maximum trans cleavage rate as measured by fluorescent signal generation) of an effector protein in a trans cleavage assay at 40° C. may be at least 50% of that at 37° C. (e.g., at least 55%, 60%, 65%, 70%, 75%, 80%, 85%, 90%, 95%, or 100% of that at 37° C.). In some embodiments, the trans cleavage activity of an effector protein in a trans cleavage assay at 40° C. may be at least 1-fold of that at 37° C. (e.g., at least 2-, 3-, 4-, 5-, 6-, 7-, 8-, 9-, or 10-fold of that at 37° C.). In some embodiments, the trans cleavage activity of an effector protein in a trans cleavage assay at 40° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some embodiments, the trans cleavage activity of an effector protein in a trans cleavage assay at 45° C. may be at least 50% of that at 37° C. (e.g., at least 55%, 60%, 65%, 70%, 75%, 80%, 85%, 90%, 95%, or 100% of that at 37° C.). In some embodiments, the trans cleavage activity of an effector protein in a trans cleavage assay at 45° C. may be at least 1-fold of that at 37° C. (e.g., at least 2-, 3-, 4-, 5-, 6-, 7-, 8-, 9-, or 10-fold of that at 37° C.). In some embodiments, the trans cleavage activity of an effector protein in a trans cleavage assay at 45° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some embodiments, the trans cleavage activity of an effector protein in a trans cleavage assay at 50° C. may be at least 50% of that at 37° C. (e.g., at least 55%, 60%, 65%, 70%, 75%, 80%, 85%, 90%, 95%, or 100% of that at 37° C.). In some embodiments, the trans cleavage activity of an effector protein in a trans cleavage assay at 50° C. may be at least 1-fold of that at 37° C. (e.g., at least 2-, 3-, 4-, 5-, 6-, 7-, 8-, 9-, or 10-fold of that at 37° C.). In some embodiments, the trans cleavage activity of an effector protein in a trans cleavage assay at 50° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some embodiments, the trans cleavage activity of an effector protein in a trans cleavage assay at 55° C. may be at least 50% of that at 37° C. (e.g., at least 55%, 60%, 65%, 70%, 75%, 80%, 85%, 90%, 95%, or 100% of that at 37° C.). In some embodiments, the trans cleavage activity of an effector protein in a trans cleavage assay at 55° C. may be at least 1-fold of that at 37° C. (e.g., at least 2-, 3-, 4-, 5-, 6-, 7-, 8-, 9-, or 10-fold of that at 37° C.). In some embodiments, the trans cleavage activity of an effector protein in a trans cleavage assay at 55° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some embodiments, the trans cleavage activity of an effector protein in a trans cleavage assay at 60° C. may be at least 50% of that at 37° C. (e.g., at least 55%, 60%, 65%, 70%, 75%, 80%, 85%, 90%, 95%, or 100% of that at 37° C.). In some embodiments, the trans cleavage activity of an effector protein in a trans cleavage assay at 60° C. may be at least 1-fold of that at 37° C. (e.g., at least 2-, 3-, 4-, 5-, 6-, 7-, 8-, 9-, or 10-fold of that at 37° C.). In some embodiments, the trans cleavage activity of an effector protein in a trans cleavage assay at 60° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some embodiments, the trans cleavage activity of an effector protein in a trans cleavage assay at 65° C. may be at least 50% of that at 37° C. (e.g., at least 55%, 60%, 65%, 70%, 75%, 80%, 85%, 90%, 95%, or 100% of that at 37° C.). In some embodiments, the trans cleavage activity of an effector protein in a trans cleavage assay at 65° C. may be at least 1-fold of that at 37° C. (e.g., at least 2-, 3-, 4-, 5-, 6-, 7-, 8-, 9-, or 10-fold of that at 37° C.). In some embodiments, the trans cleavage activity of an effector protein in a trans cleavage assay at 65° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that at 37° C.

In some embodiments, the trans cleavage activity may be measured against a negative control in a trans cleavage assay. In some embodiments, the trans cleavage activity of an effector protein against a nucleic acid in a trans cleavage assay at 37° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some embodiments, the trans cleavage activity of an effector protein against a nucleic acid in a trans cleavage assay at 37° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some embodiments, the trans cleavage activity of an effector protein against a nucleic acid in a trans cleavage assay at 40° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some embodiments, the trans cleavage activity of an effector protein against a nucleic acid in a trans cleavage assay at 40° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some embodiments, the trans cleavage activity of an effector protein against a nucleic acid in a trans cleavage assay at 45° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some embodiments, the trans cleavage activity of an effector protein against a nucleic acid in a trans cleavage assay at 45° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some embodiments, the trans cleavage activity of an effector protein against a nucleic acid in a trans cleavage assay at 50° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some embodiments, the trans cleavage activity of an effector protein against a nucleic acid in a trans cleavage assay at 50° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some embodiments, the trans cleavage activity of an effector protein against a nucleic acid in a trans cleavage assay at 55° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some embodiments, the trans cleavage activity of an effector protein against a nucleic acid in a trans cleavage assay at 55° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some embodiments, the trans cleavage activity of an effector protein against a nucleic acid in a trans cleavage assay at 60° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some embodiments, the trans cleavage activity of an effector protein against a nucleic acid in a trans cleavage assay at 60° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some embodiments, the trans cleavage activity of an effector protein against a nucleic acid in a trans cleavage assay at 65° C. may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some embodiments, the trans cleavage activity of an effector protein against a nucleic acid in a trans cleavage assay at 65° C. may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid. In some embodiments, the trans cleavage activity of an effector protein against a nucleic acid in a trans cleavage assay at 70° C., 75° C., 80° C., or more may be at least 50%, at least 55%, at least 60%, at least 65%, at least 70%, at least 75%, at least 80%, at least 85%, at least 90%, at least 95%, at least 100%, at least 1-fold, at least 2-fold, at least 3-fold, at least 4-fold, at least 5-fold, at least 6-fold, at least 7-fold, at least 8-fold, at least 9-fold, or at least 10-fold of that against a negative control nucleic acid. In some embodiments, the trans cleavage activity of an effector protein against a nucleic acid in a trans cleavage assay at 70° C., 75° C., 80° C., or more may be at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold or more of that against a negative control nucleic acid.

### Multimeric Complexes

Compositions, systems, devices, kits and methods of the present disclosure may comprise a multimeric complex or uses thereof, wherein the multimeric complex comprises one or more effector proteins that non-covalently interact with one another. A multimeric complex may comprise enhanced activity relative to the activity of any one of its effector proteins alone. For example, a multimeric complex comprising two effector proteins (e.g., in dimeric form) may comprise greater nucleic acid binding affinity and/or nuclease activity than that of either of the effector proteins provided in monomeric form. In another example, a multimeric complex comprising an effector protein and an effector partner may comprise greater nucleic acid binding affinity and/or nuclease activity than that of either of the effector protein or effector partner provided in monomeric form. A multimeric complex may have an affinity for a target sequence of a target nucleic acid and is capable of catalytic activity (e.g., cleaving, nicking, inserting or otherwise editing the nucleic acid) at or near the target sequence. A multimeric complex may have an affinity for a donor nucleic acid and is capable of catalytic activity (e.g., cleaving, nicking, editing or otherwise modifying the nucleic acid by creating cuts) at or near one or more ends of the donor nucleic acid. Multimeric complexes may be activated when complexed with a guide nucleic acid. Multimeric complexes may be activated when complexed with a target nucleic acid. Multimeric complexes may be activated when complexed with a guide nucleic acid, a target nucleic acid, and/or a donor nucleic acid. In some embodiments, the multimeric complex cleaves the target nucleic acid. In some embodiments, the multimeric complex nicks the target nucleic acid.

Various aspects of the present disclosure include compositions, devices, kits, systems and methods comprising multiple effector proteins, and uses thereof, respectively. An effector protein comprising at least 65%, at least 70%, at least 75%, at least 80%, at least 85%, at least 90%, at least 95% or 100% sequence identity to any one of the sequences of TABLE 1 may be provided with a second effector protein. Two effector proteins may target different nucleic acid sequences. Two effector proteins may target different types of nucleic acids (e.g., a first effector protein may target double- and single-stranded nucleic acids, and a second effector protein may only target single-stranded nucleic acids). It is understood that when discussing the use of more than one effector protein in compositions, systems, devices, kits and methods provided herein, the multimeric complex form is also described.

In some embodiments, multimeric complexes comprise at least one effector protein comprising an amino acid sequence with at least 65%, at least 70%, at least 75%, at least 80%, at least 85%, at least 90%, at least 95% or 100% identity to any one of the sequences of TABLE 1. In some embodiments, the multimeric complex is a dimer comprising two effector proteins of identical amino acid sequences. In some embodiments, the multimeric complex comprises a first effector protein and a second effector protein, wherein the amino acid sequence of the first effector protein is at least 90%, at least 92%, at least 94%, at least 96%, at least 98% identical, or at least 99% identical to the amino acid sequence of the second effector protein.

In some embodiments, the multimeric complex is a heterodimeric complex comprising at least two effector proteins of different amino acid sequences. In some embodiments, the multimeric complex is a heterodimeric complex comprising a first effector protein and a second effector protein, wherein the amino acid sequence of the first effector protein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% identical to the amino acid sequence of the second effector protein.

In some embodiments, a multimeric complex comprises at least two effector proteins. In some embodiments, a multimeric complex comprises more than two effector proteins. In some embodiments, a multimeric complex comprises two, three or four effector proteins. In some embodiments, at least one effector protein of the multimeric complex comprises an amino acid sequence with at least 65%, at least 70%, at least 75%, at least 80%, at least 85%, at least 90%, at least 95% or 100% identity to any one of the sequences of TABLE 1. In some embodiments, each effector protein of the multimeric complex independently comprises an amino acid sequence with at least 65%, at least 70%, at least 75%, at least 80%, at least 85%, at least 90%, at least 95% or 100% identity to any one of the sequences of TABLE 1.

### Synthesis, Isolation and Assaying

Effector proteins of the present disclosure may be synthesized, using any suitable method. In some embodiments, the effector proteins may be produced in vitro or by eukaryotic cells or by prokaryotic cells. In some embodiments, the effector proteins may be further processed by unfolding (e.g., heat denaturation, dithiothreitol reduction, etc.) and may be further refolded, using any suitable method.

Any suitable method of generating and assaying the effector proteins described herein may be used. Such methods include, but are not limited to, site-directed mutagenesis, random mutagenesis, combinatorial libraries, and other mutagenesis methods described herein (see, e.g., Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Gillman et al., Directed Evolution Library Creation: Methods and Protocols (Methods in Molecular Biology) Springer, 2nd ed (2014)). One non-limiting example of a method for preparing an effector protein is to express recombinant nucleic acids encoding the effector protein in a suitable microbial organism, such as a bacterial cell, a yeast cell, or other suitable cell, using methods well known in the art. Exemplary methods are also described in the Examples provided herein.

In some embodiments, an effector protein provided herein is an isolated effector protein. In some embodiments, the effector proteins may be isolated and purified for use in compositions, systems, devices, kits and/or methods described herein. In some embodiments, methods described here may include the step of isolating effector proteins described herein. Any suitable method to provide isolated effector proteins described herein may be used in the present disclosure, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present disclosure can be obtained using well-known recombinant methods (see, e.g., Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). The methods and conditions for biochemical purification of a polypeptide described herein can be chosen by those skilled in the art, and purification monitored, for example, by a functional assay.

In some embodiments, compositions, systems, devices, kits and methods described herein may further comprise a purification tag that can be attached to an effector protein, or a nucleic acid encoding the purification tag that can be attached to a nucleic acid encoding the effector protein as described herein. In some embodiments, the purification tag may be an amino acid sequence which can attach or bind with high affinity to a separation substrate and assist in isolating the protein of interest from its environment, which may be its biological source, such as a cell lysate. Attachment of the purification tag may be at the N or C terminus of the effector protein. Furthermore, an amino acid sequence recognized by a protease or a nucleic acid encoding for an amino acid sequence recognized by a protease, such as TEV protease or the HRV3C protease may be inserted between the purification tag and the effector protein, such that biochemical cleavage of the sequence with the protease after initial purification liberates the purification tag. Purification and/or isolation may be performed through high performance liquid chromatography (HPLC), exclusion chromatography, gel electrophoresis, affinity chromatography, or other purification technique. Non-limiting examples of purification tags are as described herein.

In some embodiments, effector proteins described herein are isolated from cell lysate. In some embodiments, the compositions described herein may comprise 20% or more by weight, 75% or more by weight, 95% or more by weight, or 99.5% or more by weight of an effector protein, related to the method of preparation of compositions described herein and its purification thereof, wherein percentages may be upon total protein content in relation to contaminants. Thus, in some embodiments, the effector protein is at least 80% pure, at least 85% pure, at least 90% pure, at least 95% pure, at least 98% pure, or at least 99% pure (e.g., free of contaminants, non-engineered proteins or other macromolecules, etc.).

### Protospacer Adjacent Motif (PAM) Sequences

In some embodiments, effector proteins cleave or nick a target nucleic acid within or near a protospacer adjacent motif (PAM) sequence of the target nucleic acid. In some embodiments, cleavage occurs within 1, 2, 3, 4, 5, 6, 7, 8, 9, 10, 15, 20, 25, 30, 35, 40, 45, or 50 nucleotides of a 5′ or 3′ terminus of a PAM sequence. In some embodiments, effector proteins described herein recognize a PAM sequence. In some embodiments, recognizing a PAM sequence comprises interacting with a sequence adjacent to the PAM. In some embodiments, a target nucleic acid comprises a target sequence that is adjacent to a PAM sequence. A target nucleic acid may comprise a PAM sequence adjacent to a sequence that is complementary to a guide nucleic acid spacer sequence. In some embodiments, effector proteins do not require a PAM sequence to cleave or a nick a target nucleic acid.

In some embodiments, a target nucleic acid is a single stranded target nucleic acid comprising a target sequence. Accordingly, in some embodiments, the single stranded target nucleic acid comprises a PAM sequence described herein that is adjacent (e.g., within 1, 2, 3, 4, 5, 6, 7, 8, 9, 10, 15, 20, 25, 30, 35, 40, 45, or 50 nucleotides) or directly adjacent to the target sequence. In some embodiments, an RNP cleaves the single stranded target nucleic acid.

In some embodiments, a target nucleic acid is a double stranded nucleic acid comprising a target strand and a non-target strand, wherein the target strand comprises a target sequence. In some embodiments, the PAM sequence is located on the target strand. In some embodiments, the PAM sequence is located on the non-target strand. In some embodiments, the PAM sequence described herein is adjacent (e.g., within 1, 2, 3, 4, 5, 6, 7, 8, 9, 10, 15, 20, 25, 30, 35, 40, 45, or 50 nucleotides) to the target sequence on the target strand or the non-target strand. In some embodiments, such a PAM described herein is directly adjacent to the target sequence on the target strand or the non-target strand. In some embodiments, an RNP cleaves the target strand or the non-target strand. In some embodiments, the RNP cleaves both, the target strand and the non-target strand. In some embodiments, an RNP recognizes the PAM sequence, and hybridizes to a target sequence of the target nucleic acid. In some embodiments, the RNP cleaves the target nucleic acid, wherein the RNP has recognized the PAM sequence and is hybridized to the target sequence.

In some embodiments, an effector protein described herein, or a multimeric complex thereof, recognizes a PAM on a target nucleic acid. In some embodiments, multiple effector proteins of the multimeric complex recognize a PAM on a target nucleic acid. In some embodiments, at least two of the multiple effector proteins recognize the same PAM sequence. In some embodiments, at least two of the multiple effector proteins recognize different PAM sequences. In some embodiments, only one effector protein of the multimeric complex recognizes a PAM on a target nucleic acid.

An effector protein of the present disclosure, or a multimeric complex thereof, may cleave or nick a target nucleic acid within or near a protospacer adjacent motif (PAM) sequence of the target nucleic acid. In some embodiments, cleavage occurs within 1, 2, 3, 4, 5, 6, 7, 8, 9 or 10 nucleotides of a 5′ or 3′ terminus of a PAM sequence.

In some embodiments, a PAM sequence comprises NNN, NNNN, NNNNN, NNNNNN, or NNNNNNN wherein each Nis independently any one of A, C, G, or T. In some embodiments, a PAM sequence comprises YTTN or TTYN wherein Y is C or T and wherein Nis A, C, G or T. In some embodiments, a PAM sequence comprises TTTN, TTCN, or CTTN wherein Nis A, C, G or T. For example, in some embodiments, a PAM sequence comprises: TTTN wherein Nis A, C, G or T; TTCN wherein Nis A, C, G or T; or CTTN wherein Nis A, C, G or T. In some embodiments, a PAM sequence provided herein comprises any one of the nucleotide sequences recited in TABLE 3. PAMs used in compositions, systems, and methods herein are further described throughout the application.

### II. Nucleic Acid Systems

### Guide Nucleic Acids

The compositions, systems, devices, kits and methods of the present disclosure may comprise a guide nucleic acid or a use thereof. Also provided herein are compositions, systems and methods that comprise at least one of: one or more guide nucleic acids and DNA molecule(s) encoding the guide nucleic acids. A person of ordinary skill in the art understands that a DNA molecule that “encodes” a nucleic acid, such as a guide nucleic acid, refers to a DNA molecule having a nucleic acid that produces an RNA molecule (e.g., a guide nucleic acid) when transcribed. It is understood that when referring to a guide nucleic acid as described herein, a DNA molecule encoding the guide nucleic acid is also described.

Guide nucleic acids are often referred to as “guide RNA.” However, a guide nucleic acid may comprise deoxyribonucleotides. The term “guide RNA,” as well as any components thereof (e.g., crRNA, repeat sequence, intermediary RNA sequence, spacer sequence, handle sequence, tracrRNA sequence, and etc.) includes guide nucleic acids comprising DNA bases, RNA bases, chemically modified nucleobases (e.g., one or more engineered modifications as described herein). A guide nucleic acid may comprise one or more deoxyribonucleotides, one or more ribonucleotides, one or more chemically modified nucleotides, or a combination thereof. A guide nucleic acid can also include a combination of DNA or RNA (e.g., RNA with a thymine base). A guide nucleic acid can also include a chemically modified nucleobase or phosphate backbone. Accordingly, guide nucleic acid, as interchangeably referred to herein as a guide RNA or gRNA, is not limited to ribonucleotides, but may comprise deoxyribonucleotides and other chemically modified nucleotides. In some embodiments, a guide nucleic acid of the present disclosure comprises one or more of the following: a) a single nucleic acid molecule; b) a DNA base; c) an RNA base; d) a modified base; e) a modified sugar; f) a modified backbone; and the like. Modifications are described herein and throughout the present disclosure (e.g., in the section entitled “Engineered Modifications”). The guide RNA may be chemically synthesized or recombinantly produced. The sequence of the guide nucleic acid, or a portion thereof, may be different from the sequence of a naturally occurring nucleic acid. Guide nucleic acids and portions thereof may be found in or identified from a CRISPR array present in the genome of a host organism or cell. The sequence of the guide nucleic acid may comprise two or more heterologous sequences.

Such nucleotide sequences described herein may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein also discloses the complementary nucleotide sequence, the reverse nucleotide sequence, and the reverse complement nucleotide sequence, any one of which can be a nucleotide sequence for use in a guide nucleic acid as described herein. In some embodiments, a guide nucleic acid sequence(s) comprises one or more nucleotide alterations at one or more positions in any one of the sequences described herein. Alternative nucleotides can be any one or more of A, C, G, T or U, or a deletion, or an insertion.

A guide nucleic acid, or at least a portion thereof, may interact with an effector protein. A guide nucleic acid, or at least a portion thereof, may bind to an effector protein. In some embodiments, where a portion of a guide nucleic acid binds to an effector protein, such binding is non-covalent binding.

In some embodiments, the guide nucleic acid comprises a CRISPR RNA (crRNA), at least a portion of which is complementary to a target sequence of a target nucleic acid. In some embodiments, a crRNA comprises a sequence that interacts with an effector protein. In some embodiments, the crRNA comprises a repeat sequence that interacts with an effector protein. In some embodiments, the guide nucleic acid comprises a trans-activating CRISPR RNA (tracrRNA) sequence that interacts with the effector protein. In some embodiments, a crRNA and tracrRNA function as two separate, unlinked molecules. In some embodiments, a crRNA and tracrRNA function as two separate, unlinked molecules, wherein the tracrRNA hybridizes with the crRNA and interacts with an effector protein. Accordingly, in this context, the tracrRNA is transacting.

On the other hand, and in some embodiments, the guide nucleic acid, compositions, devices, kits, methods, or systems described herein do not comprise a nucleotide sequence that is transactivating. In some embodiments, the guide nucleic acid does not comprise a tracrRNA. In some embodiments, the composition, devices, kits, methods, or systems described herein do not comprise a tracrRNA. In some embodiments, the guide nucleic acid comprises an intermediary RNA. In some embodiments, the guide RNA is a single guide RNA (sgRNA) (e.g., a crRNA linked to an intermediary RNA). In some embodiments, the crRNA and the intermediary RNA are covalently linked (e.g., by a phosphodiester bond), also referred to herein as a sgRNA. In some embodiments, the crRNA and the intermediary RNA are linked by one or more nucleotides. In some embodiments, a guide nucleic acid is an sgRNA.

In some embodiments, effector proteins, namely, fusion effector proteins are targeted by a guide nucleic acid (e.g., a guide RNA) to a specific location in the target nucleic acid where they exert locus-specific regulation. Non-limiting examples of locus-specific regulation include blocking RNA polymerase binding to a promoter (which selectively inhibits transcription activator function), and/or modifying local chromatin (e.g., when a fusion sequence is used that modifies the target nucleic acid or modifies a protein associated with the target nucleic acid).

The guide nucleic acid may also form complexes as described through herein. For example, a guide nucleic acid may bind or hybridize to another nucleic acid, such as target nucleic acid, or a portion thereof. The guide RNA may bind to a target nucleic acid (e.g., a single strand of a target nucleic acid) or a portion thereof, an amplicon thereof, or a portion thereof. By way of non-limiting example, a guide nucleic acid may bind to a target nucleic acid, such as DNA or RNA, from a cancer gene or gene associated with a genetic disorder, or an amplicon thereof, as described herein. In some embodiments, a guide nucleic acid may complex with an effector protein. In such embodiments, a guide nucleic acid-effector protein complex may be described herein as an RNP. In some embodiments, when in a complex, at least a portion of the complex may bind, recognize, and/or hybridize to a target nucleic acid. For example, when a guide nucleic acid and an effector protein are complexed to form an RNP, at least a portion of the guide nucleic acid hybridizes to a target sequence in a target nucleic acid. Those skilled in the art in reading the below specific examples of guide nucleic acids as used in RNPs described herein, will understand that in some embodiments, a RNP may hybridize to one or more target sequences in a target nucleic acid, thereby allowing the RNP to modify and/or recognize a target nucleic acid or sequence contained therein (e.g., PAM) or to modify and/or recognize non-target sequences depending on the guide nucleic acid, and in some embodiments, the effector protein, used.

In some embodiments, a guide nucleic acid may comprise or form intramolecular secondary structure (e.g., hairpins, stem-loops, etc.). In some embodiments, a guide nucleic acid comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the guide nucleic acid comprises a pseudoknot (e.g., a secondary structure comprising a stem, at least partially, hybridized to a second stem or half-stem secondary structure). An effector protein may recognize a guide nucleic acid comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the guide nucleic acid comprises at least 2, at least 3, at least 4, or at least 5 stem regions.

In some embodiments, the compositions, systems, and methods of the present disclosure comprise two or more guide nucleic acids (e.g., 2, 3, 4, 5, 6, 7, 9, 10 or more guide nucleic acids), and/or uses thereof. Multiple guide nucleic acids may target an effector protein to different locations in the target nucleic acid by hybridizing to different target sequences. In some embodiments, a first guide nucleic acid may hybridize within a location of the target nucleic acid that is different from where a second guide nucleic acid may hybridize the target nucleic acid. In some embodiments, the first loci and the second loci of the target nucleic acid may be located at least 1, at least 10, at least 20, at least 30, at least 40, at least 50, at least 60, at least 70, at least 80, at least 90 or at least 100 nucleotides apart. In some embodiments, the first loci and the second loci of the target nucleic acid may be located between 100 and 200, 200 and 300, 300 and 400, 400 and 500, 500 and 600, 600 and 700, 700 and 800, 800 and 900 or 900 and 1000 nucleotides apart.

In some embodiments, the first loci and/or the second loci of the target nucleic acid are located in an intron of a gene. In some embodiments, the first loci and/or the second loci of the target nucleic acid are located in an exon of a gene. In some embodiments, the first loci and/or the second loci of the target nucleic acid span an exon-intron junction of a gene. In some embodiments, the first portion and/or the second portion of the target nucleic acid are located on either side of an exon and cutting at both sites results in deletion of the exon. In some embodiments, composition, systems, and methods comprise a donor nucleic acid that may be inserted in replacement of a deleted or cleaved sequence of the target nucleic acid. In some embodiments, compositions, systems, and methods comprising multiple guide nucleic acids or uses thereof comprise multiple effector proteins, wherein the effector proteins may be identical, non-identical, or combinations thereof.

In some embodiments, an effector protein cleaves a precursor RNA (“pre-crRNA”) to produce a guide RNA, also referred to as a “mature guide RNA.” An effector protein that cleaves pre-crRNA to produce a mature guide RNA is said to have pre-crRNA processing activity. In some embodiments, a repeat sequence of a guide RNA comprises mutations or truncations relative to respective regions in a corresponding pre-crRNA.

The guide nucleic acid may comprise a first region complementary to a target nucleic acid (FR1) and a second region that is not complementary to the target nucleic acid (FR2). In some embodiments, FR1 is located 5′ to FR2 (FR1-FR2). In some embodiments, FR2 is located 5′ to FR1 (FR2-FR1). In some embodiments, the FR2 comprises one or more repeat sequences or intermediary sequence. In some embodiments, an effector protein binds to at least a portion of the FR2. In some embodiments, the FR1 comprises a spacer sequence, wherein the spacer sequence can interact in a sequence-specific manner with (e.g., has complementarity with, or can hybridize to a target sequence in) a target nucleic acid.

In some embodiments, the guide nucleic acid comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In general, a guide nucleic acid comprises at least linked nucleotides. In some embodiments, a guide nucleic acid comprises at least 25 linked nucleotides. A guide nucleic acid may comprise 10 to 50 linked nucleotides. In some embodiments, the guide nucleic acid comprises or consists essentially of about 12 to about 80 linked nucleotides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleotides. In some embodiments, the guide nucleic acid has about 10 to about 60, about 20 to about 50, or about 30 to about 40 linked nucleotides.

In some embodiments, a guide nucleic acid comprises at least 10, 11, 12, 13, 14, 15, 16, 17, 18, 19, 20, 21, 22, 23, 24, 25, 26, 27, 28, 29, or 30 contiguous nucleotides that are complementary to a eukaryotic sequence. Such a eukaryotic sequence is a nucleotide sequence that is present in a host eukaryotic cell. Such a nucleotide sequence is distinguished from nucleotide sequences present in other host cells, such as prokaryotic cells, or viruses. Said sequences present in a eukaryotic cell can be located in a gene, an exon, an intron, a non-coding (e.g., promoter or enhancer) region, a selectable marker, tag, signal, and the like. In some embodiments, a target sequence is a eukaryotic sequence.

In some embodiments, guide nucleic acids comprise additional elements that contribute additional functionality (e.g., stability, heat resistance, etc.) to the guide nucleic acid. Such elements may be one or more nucleotide alterations, nucleotide sequences, intermolecular secondary structures, or intramolecular secondary structures (e.g., one or more hair pin regions, one or more bulges, etc.).

In some embodiments, guide nucleic acids comprise one or more linkers connecting different nucleotide sequences as described herein. A linker may comprise 1, 2, 3, 4, 5, 6, 7, 8, 9, 10, or more nucleotides. A linker may be any suitable linker, examples of which are described herein.

In some embodiments, the guide nucleic acid comprises a nucleotide sequence as described herein (e.g., TABLE 4, TABLE 5, TABLE 6, TABLE 9, TABLE 10, and SEQ ID NO: 32). Such nucleotide sequences described herein (e.g., TABLE 4, TABLE 5, TABLE 6, TABLE 9, TABLE 10, and SEQ ID NO: 32) may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein (e.g., TABLE 4, TABLE 5, TABLE 6, TABLE 9, TABLE 10, and SEQ ID NO: 32) also discloses the complementary nucleotide sequence, the reverse nucleotide sequence, and the reverse complement nucleotide sequence, any one of which can be a nucleotide sequence for use in a guide nucleic acid as described herein.

In some embodiments, a spacer sequence comprises a nucleotide sequence that hybridizes to a target sequence of a target nucleic acid. In some embodiments, the spacer sequence comprises a nucleotide sequence as described herein (e.g., TABLE 9 and SEQ ID NO: 32). Such nucleotide sequences described herein (e.g., TABLE 9 and SEQ ID NO: 32) may be described as a nucleotide sequence of either DNA or RNA, however, no matter the form the sequence is described, it is readily understood that such nucleotide sequences can be revised to be RNA or DNA, as needed, for describing a sequence within a spacer sequence itself or the sequence that encodes a spacer sequence, such as a nucleotide sequence described herein for a vector. Similarly, disclosure of the nucleotide sequences described herein (e.g., TABLE 9 and SEQ ID NO: 32) also discloses the complementary nucleotide sequence, the reverse nucleotide sequence, and the reverse complement nucleotide sequence, any one of which can be a nucleotide sequence for use in a spacer sequence as described herein.

### Repeat Sequence

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that may interact with an effector protein. In some embodiments, a repeat sequence is connected to another sequence of a guide nucleic acid, such as an intermediary sequence, that is capable of non-covalently interacting with an effector protein. In some embodiments, a repeat sequence includes a nucleotide sequence that is capable of forming a guide nucleic acid-effector protein complex (e.g., a RNP complex). A repeat sequence may also be referred to as a repeat region, which is understood to be equivalent to a repeat sequence as described herein, and thus the terms are used interchangeably.

In some embodiments, the repeat sequence is between 10 and 50, 12 and 48, 14 and 46, 16 and 44, and 18 and 42 nucleotides in length. The repeat sequence may also be referred to as a “protein-binding segment.” Typically, the repeat sequence is adjacent to the spacer sequence. For example, a guide RNA that interacts with an effector protein comprises a repeat sequence that is 5′ of the spacer sequence. In some embodiments, a repeat sequence is followed by a spacer sequence in the 5′ to 3′ direction. In some embodiments, a repeat sequence is preceded by a spacer sequence in the 5′ to 3′ direction. In some embodiments, a repeat sequence is adjacent to an intermediary sequence. In some embodiments, a repeat sequence is 3′ to an intermediary sequence. In some embodiments, an intermediary sequence is followed by a repeat sequence, which is followed by a spacer sequence in the 5′ to 3′ direction. In some embodiments, a repeat sequence is linked to a spacer sequence and/or an intermediary sequence. In some embodiments, a guide nucleic acid comprises a repeat sequence linked to a spacer sequence and/or to an intermediary sequence, which may be a direct link or by any suitable linker, examples of which are described herein.

In some embodiments, guide nucleic acids comprise more than one repeat sequence (e.g., two or more, three or more, or four or more repeat sequences). In some embodiments, a guide nucleic acid comprises more than one repeat sequence separated by another sequence of the guide nucleic acid. For example, in some embodiments, a guide nucleic acid comprises two repeat sequences, wherein the first repeat sequence is followed by a spacer sequence, and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence may include a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is, at least partially, complementary. In some embodiments, such sequences may have 65% to 100% complementarity (e.g., 65%, 70%, 75%, 80%, 85%, 90%, 95%, or 100% complementarity). In some embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

In some embodiments, the repeat sequence comprises a nucleotide sequence that interacts with the effector protein. In some embodiments, the repeat sequence comprises a nucleotide sequence as described herein (e.g., TABLE 4). Such nucleotide sequences described herein (e.g., TABLE 4) may be described as a nucleotide sequence of either DNA or RNA, however, no matter the form the sequence is described, it is readily understood that such nucleotide sequences can be revised to be RNA or DNA, as needed, for describing a sequence within a repeat sequence itself or the sequence that encodes a repeat sequence, such as a nucleotide sequence described herein for a vector. Similarly, disclosure of the nucleotide sequences described herein (e.g., TABLE 4) also discloses the complementary nucleotide sequence, the reverse nucleotide sequence, and the reverse complement nucleotide sequence, any one of which can be a nucleotide sequence for use in a repeat sequence as described herein.

In some embodiments, the repeat sequence comprises a nucleotide sequence that is at least 65%, 70%, 75%, 80%, 85%, 90%, 95%, 97%, 99% or 100% identical to any one of the sequences as set forth in TABLE 4. In some embodiments, a repeat sequence comprises a nucleotide sequence that is at least 65% identical to any one of the sequences as set forth in TABLE 4. In some embodiments, a repeat sequence comprises a nucleotide sequence that is at least 70% identical to any one of the sequences as set forth in TABLE 4. In some embodiments, a repeat sequence comprises a nucleotide sequence that is at least 75% identical to any one of the sequences as set forth in TABLE 4. In some embodiments, a repeat sequence comprises a nucleotide sequence that is at least 80% identical to any one of the sequences as set forth in TABLE 4. In some embodiments, a repeat sequence comprises a nucleotide sequence that is at least 85% identical to any one of the sequences as set forth in TABLE 4. In some embodiments, a repeat sequence comprises a nucleotide sequence that is at least 90% identical to any one of the sequences as set forth in TABLE 4. In some embodiments, a repeat sequence comprises a nucleotide sequence that is at least 95% identical to any one of the sequences as set forth in TABLE 4. In some embodiments, a repeat sequence comprises a nucleotide sequence that is at least 97% identical to any one of the sequences as set forth in TABLE 4. In some embodiments, a repeat sequence comprises a nucleotide sequence that is at least 99% identical to any one of the sequences as set forth in TABLE 4. In some embodiments, a repeat sequence comprises a nucleotide sequence that is identical to any one of the sequences as set forth in TABLE 4.

In some embodiments, a repeat sequence comprises one or more nucleotide alterations at one or more positions in the sequence recited in TABLE 4. Alternative nucleotides can be any one or more of A, C, G, T or U, or a deletion, or an insertion.

### Spacer Sequence

Guide nucleic acids described herein may comprise one or more spacer sequences. In some embodiments, a spacer sequence is capable of hybridizing to a target sequence of a target nucleic acid. In some embodiments, a spacer sequence comprises a nucleotide sequence that is, at least partially, hybridizable to an equal length of a sequence (e.g., a target sequence) of a target nucleic acid. Exemplary hybridization conditions are described herein. In some embodiments, the spacer sequence may function to direct an RNP complex comprising the guide nucleic acid to the target nucleic acid for detection and/or modification. The spacer sequence may function to direct a RNP to the target nucleic acid for detection and/or modification. A spacer sequence may be complementary to a target sequence that is adjacent to a PAM that is recognizable by an effector protein described herein. A spacer sequence may also be referred to as a spacer region, which is understood to be equivalent to a spacer sequence as described herein, and thus the terms are used interchangeably.

The spacer sequence may comprise complementarity with (e.g., hybridize to) a target sequence of a target nucleic acid. In some embodiments, the spacer sequence is 15-28 linked nucleotides in length. In some embodiments, the spacer sequence is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides in length. In some embodiments, the spacer sequence is 18-24 linked nucleotides in length. In some embodiments, the spacer sequence is at least 15 linked nucleotides in length. In some embodiments, the spacer sequence is at least 16, 18, 20, or 22 linked nucleotides in length. In some embodiments, the spacer sequence comprises at least 10, 11, 12, 13, 14, 15, 16, 17, 18, 19, 20, 21, 22, 23, 24, 25, 26, 27, 28, 29, or 30 nucleotides. In some embodiments, the spacer sequence is at least 17 linked nucleotides in length. In some embodiments, the spacer sequence is at least 18 linked nucleotides in length. In some embodiments, the spacer sequence is at least 20 linked nucleotides in length.

In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. Linkers may be any suitable linker. In some embodiments, the spacer sequence(s) and the repeat sequence(s) of the guide nucleic acid are present in separate molecules, which are joined to one another by base pairing interactions.

In some embodiments, the spacer sequence is at least 80%, at least 85%, at least 90%, at least 95% or 100% complementary to a target sequence of the target nucleic acid. In some embodiments, the spacer sequence is 100% complementary to the target sequence of the target nucleic acid. In some embodiments, the spacer sequence comprises at least 15 contiguous nucleobases that are complementary to the target nucleic acid. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are complementary to the target sequence.

A spacer sequence is capable of hybridizing to an equal length portion of a target nucleic acid (e.g., a target sequence). In some embodiments, a target nucleic acid, such as DNA or RNA, may be a cancer gene or gene associated with a genetic disorder, or an amplicon thereof, as described herein. In some embodiments, a target nucleic acid is a gene selected from TABLE 7. In some embodiments, a spacer sequence comprises a sequence that is at least 65%, at least 70%, at least 75%, at least 80%, at least 85%, at least 90%, at least 95% or 100% complementary to a target sequence of a target nucleic acid selected from TABLE 7. In some embodiments, a target nucleic acid is a nucleic acid associated with a disease or syndrome set forth in TABLE 8. In some embodiments, a spacer sequence comprises a sequence that is at least 65%, at least 70%, at least 75%, at least 80%, at least 85%, at least 90%, at least 95% or 100% complementary to a target sequence of a target nucleic acid associated with a disease or syndrome set forth in TABLE 8. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are capable of hybridizing to the target sequence.

It is understood that the sequence of a spacer sequence need not be 100% complementary to that of a target sequence of a target nucleic acid to hybridize or hybridize specifically to the target sequence. The guide nucleic acid may comprise at least one uracil between nucleic acid residues 5 to 20 of the spacer sequence that is not complementary to the corresponding nucleoside of the target sequence. The guide nucleic acid may comprise at least one uracil between nucleic acid residues 5 to 9, 10 to 14, or 15 to 20 of the spacer sequence that is not complementary to the corresponding nucleoside of the target sequence. In some embodiments, the region of the target nucleic acid that is complementary to the spacer sequence comprises an epigenetic modification or a post-transcriptional modification. In some embodiments, the epigenetic modification comprises an acetylation, methylation, or thiol modification.

In some embodiments, a spacer sequence comprises a nucleotide sequence that is at least 65%, 70%, 75%, 80%, 85%, 90%, 95%, 97%, 99% or 100% identical to any one of the sequences as set forth in TABLE 9, or SEQ ID NO: 32. In some embodiments, a spacer sequence comprises a nucleotide sequence that is at least 65% identical to any one of the sequences set forth in TABLE 9 or to SEQ ID NO: 32. In some embodiments, a spacer sequence comprises a nucleotide sequence that is at least 70% identical to any one of the sequences set forth in TABLE 9 or to SEQ ID NO: 32. In some embodiments, a spacer sequence comprises a nucleotide sequence that is at least 75% identical to any one of the sequences set forth in TABLE 9 or to SEQ ID NO: 32. In some embodiments, a spacer sequence comprises a nucleotide sequence that is at least 80% identical to any one of the sequences set forth in TABLE 9 or to SEQ ID NO: 32. In some embodiments, a spacer sequence comprises a nucleotide sequence that is at least 85% identical to any one of the sequences set forth in TABLE 9 or to SEQ ID NO: 32. In some embodiments, a spacer sequence comprises a nucleotide sequence that is at least 90% identical to any one of the sequences set forth in TABLE 9 or to SEQ ID NO: 32. In some embodiments, a spacer sequence comprises a nucleotide sequence that is at least 95% identical to any one of the sequences set forth in TABLE 9 or to SEQ ID NO: 32. In some embodiments, a spacer sequence comprises a nucleotide sequence that is at least 97% identical to any one of the sequences set forth in TABLE 9 or to SEQ ID NO: 32. In some embodiments, a spacer sequence comprises a nucleotide sequence that is at least 99% identical to any one of the sequences set forth in TABLE 9 or to SEQ ID NO: 32. In some embodiments, a spacer sequence comprises a nucleotide sequence that is identical to any one of the sequences set forth in TABLE 9 or to SEQ ID NO: 32.

Spacer sequences are further described throughout herein, for example, in the Examples section.

### Linker for Nucleic Acids

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises more than one linker. In some embodiments, at least two of the more than one linker are the same. In some embodiments, at least two of the more than one linker are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′.

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

### Intermediary Sequence

Guide nucleic acids described herein may comprise one or more intermediary sequences. In general, an intermediary sequence used in the present disclosure is not transactivated or transactivating. An intermediary sequence may also be referred to as an intermediary RNA, although it may comprise deoxyribonucleotides instead of or in addition to ribonucleotides, and/or modified bases. In general, the intermediary sequence non-covalently binds to an effector protein. In some embodiments, the intermediary sequence forms a secondary structure, for example in a cell, and an effector protein binds the secondary structure.

In some embodiments, a length of the intermediary sequence is at least 30, 50, 70, 90, 110, 130, 150, 170, 190, or 210 linked nucleotides. In some embodiments, a length of the intermediary sequence is not greater than 30, 50, 70, 90, 110, 130, 150, 170, 190, or 210 linked nucleotides. In some embodiments, the length of the intermediary sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

An intermediary sequence may also comprise or form a secondary structure (e.g., one or more hairpin loops) that facilitates the binding of an effector protein to a guide nucleic acid and/or modification activity of an effector protein on a target nucleic acid (e.g., a hairpin region). An intermediary sequence may comprise from 5′ to 3′, a 5′ region, a hairpin region, and a 3′ region. In some embodiments, the 5′ region may hybridize to the 3′ region. In some embodiments, the 5′ region of the intermediary sequence does not hybridize to the 3′ region.

In some embodiments, the hairpin region may comprise a first sequence, a second sequence that is reverse complementary to the first sequence, and a stem-loop linking the first sequence and the second sequence. In some embodiments, an intermediary sequence comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, an intermediary sequence comprises a pseudoknot (e.g., a secondary structure comprising a stem at least partially hybridized to a second stem or half-stem secondary structure). An effector protein may interact with an intermediary sequence comprising a single stem region or multiple stem regions. In some embodiments, the nucleotide sequences of the multiple stem regions are identical to one another. In some embodiments, the nucleotide sequences of at least one of the multiple stem regions is not identical to those of the others. In some embodiments, an intermediary sequence comprises 1, 2, 3, 4, 5 or more stem regions.

### Handle Sequence

Guide nucleic acids described herein may comprise one or more handle sequences. In some embodiments, the handle sequence comprises an intermediary sequence. In such embodiments, at least a portion of an intermediary sequence non-covalently bonds with an effector protein. In some embodiments, the intermediary sequence is at the 3′-end of the handle sequence. In some embodiments, the intermediary sequence is at the 5′-end of the handle sequence. Additionally, or alternatively, in some embodiments, the handle sequence further comprises one or more of linkers and repeat sequences. In such embodiments, at least a portion of an intermediary sequence, or both of at least a portion of the intermediary sequence and at least a portion of repeat sequence, non-covalently interacts with an effector protein. In some embodiments, an intermediary sequence and repeat sequence are directly linked (e.g., covalently linked, such as through a phosphodiester bond). In some embodiments, the intermediary sequence and repeat sequence are linked by a suitable linker, examples of which are provided herein. In some embodiments, the linker comprises a sequence of 5′-GAAA-3′. In some embodiments, the intermediary sequence is 5′ to the repeat sequence. In some embodiments, the intermediary sequence is 5′ to the linker. In some embodiments, the intermediary sequence is 3′ to the repeat sequence. In some embodiments, the intermediary sequence is 3′ to the linker. In some embodiments, the repeat sequence is 3′ to the linker. In some embodiments, the repeat sequence is 5′ to the linker. In general, a single guide nucleic acid, also referred to as a single guide RNA (sgRNA), comprises a handle sequence comprising an intermediary sequence, and optionally one or more of a repeat sequence and a linker.

A handle sequence may comprise or form a secondary structure (e.g., one or more hairpin loops) that facilitates the binding of an effector protein to a guide nucleic acid and/or modification activity of an effector protein on a target nucleic acid (e.g., a hairpin region). In some embodiments, handle sequences comprise a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the handle sequence comprises a pseudoknot (e.g., a secondary structure comprising a stem at least partially hybridized to a second stem or half-stem secondary structure). An effector protein may recognize a handle sequence comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the handle sequence comprises at least 2, at least 3, at least 4, or at least 5 stem regions.

In some embodiments, a length of the handle sequence is at least 30, 50, 70, 90, 110, 130, 150, 170, 190, or 210 linked nucleotides. In some embodiments, a length of the handle sequence is not greater than 30, 50, 70, 90, 110, 130, 150, 170, 190, or 210 linked nucleotides. In some embodiments, the length of the handle sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

### A Single Nucleic Acid System

In some embodiments, compositions, systems, devices, kits and methods described herein comprise a single nucleic acid system comprising a guide nucleic acid or a nucleotide sequence encoding the guide nucleic acid, and one or more effector proteins or a nucleotide sequence encoding the one or more effector proteins. In some embodiments, a FR2 of the guide nucleic acid non-covalently interacts with the one or more polypeptides described herein. In some embodiments, a FR1 of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a crRNA or a sgRNA.

crRNA

In some embodiments, a guide nucleic acid comprises a crRNA. In general, a crRNA comprises a spacer sequence that hybridizes to a target sequence of a target nucleic acid, and a repeat sequence that interacts with the effector protein. In some embodiments, the guide nucleic acid is the crRNA. In general, a crRNA comprises a first region (FR1) and a second region (FR2), wherein the FR2 of the crRNA comprises a repeat sequence, and the FR1 of the crRNA comprises a spacer sequence. In some embodiments, the repeat sequence and the spacer sequences are directly connected to each other (e.g., covalent bond (phosphodiester bond)). In some embodiments, the repeat sequence and the spacer sequence are connected by a linker.

In some embodiments, a crRNA is useful as a single nucleic acid system for compositions, methods, and systems described herein or as part of a single nucleic acid system for compositions, methods, and systems described herein. In some embodiments, a crRNA is useful as part of a single nucleic acid system for compositions, methods, and systems described herein. In such embodiments, a single nucleic acid system comprises a guide nucleic acid comprising a crRNA wherein, a repeat sequence of a crRNA is capable of connecting a crRNA to an effector protein. In some embodiments, a single nucleic acid system comprises a guide nucleic acid comprising a crRNA linked to another nucleotide sequence that is capable of being non-covalently bond by an effector protein. In such embodiments, a repeat sequence of a crRNA can be linked to an intermediary sequence. In some embodiments, a single nucleic acid system comprises a guide nucleic acid comprising a crRNA and an intermediary sequence.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

In some embodiments, a crRNA comprises a nucleotide sequence that is at least 65%, 70%, 75%, 80%, 85%, 90%, 95%, 97%, 99% or 100% identical to any one of the sequences set forth in TABLE 5. In some embodiments, a crRNA comprises a nucleotide sequence that is at least 65% identical to any one of the sequences set forth in TABLE 5. In some embodiments, a crRNA comprises a nucleotide sequence that is at least 70% identical to any one of the sequences set forth in TABLE 5. In some embodiments, a crRNA comprises a nucleotide sequence that is at least 75% identical to any one of the sequences set forth in TABLE 5. In some embodiments, a crRNA comprises a nucleotide sequence that is at least 80% identical to any one of the sequences set forth in TABLE 5. In some embodiments, a crRNA comprises a nucleotide sequence that is at least 85% identical to any one of the sequences set forth in TABLE 5. In some embodiments, a crRNA comprises a nucleotide sequence that is at least 90% identical to any one of the sequences set forth in TABLE 5. In some embodiments, a crRNA comprises a nucleotide sequence that is at least 95% identical to any one of the sequences set forth in TABLE 5. In some embodiments, a crRNA comprises a nucleotide sequence that is at least 97% identical to any one of the sequences set forth in TABLE 5. In some embodiments, a crRNA comprises a nucleotide sequence that is at least 99% identical to any one of the sequences set forth in TABLE 5. In some embodiments, a crRNA comprises a nucleotide sequence that is identical to any one of the sequences set forth in TABLE 5.

sgRNA

In some embodiments, a guide nucleic acid comprises a sgRNA (a “single guide nucleic acid” or a “single guide RNA”). In some embodiments, a sgRNA in the context of a single nucleic acid system, describes a guide nucleic acid, wherein the guide nucleic acid is a single polynucleotide chain having all the required sequence for a functional complex with an effector protein (e.g., being bound by an effector protein, including in some embodiments activating the effector protein, and hybridizing to a target nucleic acid, without the need for a second nucleic acid molecule). For example, an sgRNA can have two or more linked guide nucleic acid components (e.g., an intermediary sequence, a repeat sequence, a spacer sequence and optionally a linker, or a handle sequence and a spacer sequence). In some embodiments, a guide nucleic acid is a sgRNA. In some embodiments, a sgRNA comprises a first region (FR) and a second region (SR), wherein the FR comprises a handle sequence and the SR comprises a spacer sequence. In some embodiments, the handle sequence and the spacer sequences are directly connected to each other (e.g., covalent bond (phosphodiester bond)). In some embodiments, the handle sequence and the spacer sequence are connected by a linker.

In some embodiments, a sgRNA comprises one or more of one or more of a handle sequence, an intermediary sequence, a crRNA, a repeat sequence, a spacer sequence, a linker, or combinations thereof. For example, a sgRNA comprises a handle sequence and a spacer sequence; an intermediary sequence and an crRNA; an intermediary sequence, a repeat sequence and a spacer sequence; and the like.

In some embodiments, a sgRNA comprises an intermediary sequence and an crRNA. In some embodiments, an intermediary sequence is 5′ to a crRNA in an sgRNA. In some embodiments, a sgRNA comprises a linked intermediary sequence and crRNA. In some embodiments, an intermediary sequence and a crRNA are linked in an sgRNA directly (e.g., covalently linked, such as through a phosphodiester bond) In some embodiments, an intermediary sequence and a crRNA are linked in an sgRNA by any suitable linker, examples of which are provided herein.

In some embodiments, a sgRNA comprises a handle sequence and a spacer sequence. In some embodiments, a handle sequence is 5′ to a spacer sequence in an sgRNA. In some embodiments, a sgRNA comprises a linked handle sequence and spacer sequence. In some embodiments, a handle sequence and a spacer sequence are linked in an sgRNA directly (e.g., covalently linked, such as through a phosphodiester bond) In some embodiments, a handle sequence and a spacer sequence are linked in an sgRNA by any suitable linker, examples of which are provided herein.

In some embodiments, a sgRNA comprises an intermediary sequence, a repeat sequence, and a spacer sequence. In some embodiments, an intermediary sequence is 5′ to a repeat sequence in an sgRNA. In some embodiments, a sgRNA comprises a linked intermediary sequence and repeat sequence. In some embodiments, an intermediary sequence and a repeat sequence are linked in an sgRNA directly (e.g., covalently linked, such as through a phosphodiester bond). In some embodiments, an intermediary sequence and a repeat sequence are linked in an sgRNA by any suitable linker, examples of which are provided herein. In some embodiments, a repeat sequence is 5′ to a spacer sequence in an sgRNA. In some embodiments, a sgRNA comprises a linked repeat sequence and spacer sequence. In some embodiments, a repeat sequence and a spacer sequence are linked in an sgRNA directly (e.g, covalently linked, such as through a phosphodiester bond) In some embodiments, a repeat sequence and a spacer sequence are linked in an sgRNA by any suitable linker, examples of which are provided herein.

In some embodiments, a guide nucleic acid comprises an sgRNA. In some embodiments, a guide nucleic acid is an sgRNA. In some embodiments, a sgRNA comprises a crRNA. In some embodiments, a sgRNA is a crRNA. In some embodiments, a sgRNA comprises a nucleotide sequence as described herein (e.g., TABLE 6). Such nucleotide sequences described herein (e.g., TABLE 6) may be described as a nucleotide sequence of either DNA or RNA, however, no matter the form the sequence is described, it is readily understood that such nucleotide sequences can be revised to be RNA or DNA, as needed, for describing a sequence within an sgRNA itself or the sequence that encodes an sgRNA, such as a nucleotide sequence described herein for a vector. Similarly, disclosure of the nucleotide sequences described herein (e.g., TABLE 6) also discloses the complementary nucleotide sequence, the reverse nucleotide sequence, and the reverse complement nucleotide sequence, any one of which can be a nucleotide sequence for use in a sgRNA as described herein.

In some embodiments, an sgRNA comprises a nucleotide sequence that is at least 65%, 70%, 75%, 80%, 85%, 90%, 95%, 97%, 99% or 100% identical to any one of the sequences as set forth in TABLE 6. In some embodiments, an sgRNA comprises a nucleotide sequence that is at least 65% identical to any one of the sequences as set forth in TABLE 6. In some embodiments, an sgRNA comprises a nucleotide sequence that is at least 70% identical to any one of the sequences as set forth in TABLE 6. In some embodiments, an sgRNA comprises a nucleotide sequence that is at least 75% identical to any one of the sequences as set forth in TABLE 6. In some embodiments, an sgRNA comprises a nucleotide sequence that is at least 80% identical to any one of the sequences as set forth in TABLE 6. In some embodiments, an sgRNA comprises a nucleotide sequence that is at least 85% identical to any one of the sequences as set forth in TABLE 6. In some embodiments, an sgRNA comprises a nucleotide sequence that is at least 90% identical to any one of the sequences as set forth in TABLE 6. In some embodiments, an sgRNA comprises a nucleotide sequence that is at least 95% identical to any one of the sequences as set forth in TABLE 6. In some embodiments, an sgRNA comprises a nucleotide sequence that is at least 97% identical to any one of the sequences as set forth in TABLE 6. In some embodiments, an sgRNA comprises a nucleotide sequence that is at least 99% identical to any one of the sequences as set forth in TABLE 6. In some embodiments, an sgRNA comprises a nucleotide sequence that is identical to any one of the sequences as set forth in TABLE 6.

### A Dual Nucleic Acid System

In some embodiments, compositions, systems, devices, kits and methods described herein comprise a dual nucleic acid system comprising a crRNA or a nucleotide sequence encoding the crRNA, a tracrRNA or a nucleotide sequence encoding the tracrRNA, and one or more effector protein or a nucleotide sequence encoding the one or more effector protein, wherein the crRNA and the tracrRNA are separate, unlinked molecules, wherein a repeat hybridization region of the tracrRNA is capable of hybridizing with an equal length portion of the crRNA to form a tracrRNA-crRNA duplex, wherein the equal length portion of the crRNA does not include a spacer sequence of the crRNA, and wherein the spacer sequence is capable of hybridizing to a target sequence of the target nucleic acid. In the dual nucleic acid system having a complex of the guide nucleic acid, tracrRNA, and the effector protein, the effector protein is transactivated by the tracrRNA. In other words, activity of effector protein requires binding to a tracrRNA molecule.

tracrRNA

A tracrRNA can refer to a nucleic acid that comprises a sequence that is capable of being bound (e.g., non-covalently) by an effector protein. A tracrRNA may include chemically modified nucleotides, or any combination of deoxyribonucleotides, ribonucleotides, and chemically modified nucleotides.

The tracrRNA sequence may be linked to a crRNA to form a composite gRNA. In some embodiments, the crRNA and the tracrRNA sequence are provided as a single nucleic acid (e.g., covalently linked). In some embodiments, the crRNA and tracrRNA sequence are linked by a phosphodiester bond. In some embodiments, the crRNA and tracrRNA sequence are linked by one or more linked nucleotides.

In some embodiments, a guide nucleic acid may comprise a crRNA, a short-complementarity untranslated RNA (scoutRNA), a tracrRNA, or any combination thereof. In some embodiments, compositions, devices, kits, methods, and systems described herein comprise a tracrRNA that is separate from, but forms a complex with a crRNA to form a gRNA system. In some embodiments, such a system is a dual nucleic acid system.

In some embodiments, the crRNA and the tracrRNA are separate polynucleotides. A tracrRNA and/or tracrRNA-crRNA duplex may form a secondary structure that facilitates the binding of an effector protein to a tracrRNA or a tracrRNA-crRNA. In some embodiments, the secondary structure modifies activity of the effector protein on a target nucleic acid.

A tracrRNA may comprise a repeat hybridization region and a hairpin region. The repeat hybridization region may hybridize to all or part of the sequence of the repeat of a crRNA. TracrRNAs may comprise a sequence that hybridizes to a portion of a crRNA, which may be referred to herein as a repeat hybridization sequence. In some embodiments, tracrRNAs are covalently linked to a crRNA. A tracrRNA may be separate from, but form a complex with a guide nucleic acid and an effector protein. A tracrRNA may be attached (e.g., covalently) by an artificial linker to a guide nucleic acid. A tracrRNA may include a nucleotide sequence that hybridizes with a portion of a guide nucleic acid.

In some embodiments, a tracrRNA may form a secondary structure (e.g., one or more hairpin loops) that facilitates the: binding of an effector protein to a guide nucleic acid and/or modification activity of an effector protein on a target nucleic acid. The repeat hybridization region may be positioned 3′ of the hairpin region. The hairpin region may comprise a first sequence, a second sequence that is reverse complementary to the first sequence, and a stem-loop linking the first sequence and the second sequence.

In some embodiments, tracrRNAs comprise a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the tracrRNA comprises a pseudoknot (e.g., a secondary structure comprising a stem at least partially hybridized to a second stem or half-stem secondary structure). An effector protein may recognize a tracrRNA comprising multiple stem regions. In some embodiments, the amino acid sequences of the multiple stem regions are identical to one another. In some embodiments, the amino acid sequences of at least one of the multiple stem regions is not identical to those of the others. In some embodiments, the tracrRNA comprises at least 2, at least 3, at least 4, or at least 5 stem regions.

In some embodiments, the length of a tracrRNA is not greater than 50, 56, 68, 71, 73, 95, or 105 linked nucleotides. In some embodiments, the length of a tracrRNA is about 30 to about 120 linked nucleotides. In some embodiments, the length of a tracrRNA is about 50 to about 105, about 50 to about 95, about 50 to about 73, about 50 to about 71, about 50 to about 68, or about 50 to about 56 linked nucleotides. In some embodiments, the length of a tracrRNA is 56 to 105 linked nucleotides, from 56 to 105 linked nucleotides, 68 to 105 linked nucleotides, 71 to 105 linked nucleotides, 73 to 105 linked nucleotides, or 95 to 105 linked nucleotides. In some embodiments, the length of a tracrRNA is 40 to 60 nucleotides. In some embodiments, the length of a tracrRNA is 50, 56, 68, 71, 73, 95, or 105 linked nucleotides. In some embodiments, the length of a tracrRNA is 50 nucleotides.

An exemplary tracrRNA may comprise, from 5′ to 3′, a 5′ region, a hairpin region, a repeat hybridization region, and a 3′ region. In some embodiments, the 5′ region may hybridize to the 3′ region. In some embodiments, the 5′ region does not hybridize to the 3′ region. In some embodiments, the 3′ region is covalently linked to the crRNA (e.g., through a phosphodiester bond). In some embodiments, a tracrRNA may comprise an unhybridized region at the 3′ end of the tracrRNA. The unhybridized region may have a length of about 1, about 2, about 3, about 4, about 5, about 6, about 7, about 8, about 9, about 10, about 12, about 14, about 16, about 18, or about 20 linked nucleotides. In some embodiments, the length of the un-hybridized region is 0 to 20 linked nucleotides.

In some embodiments, the guide RNA does not comprise a tracrRNA. In some embodiments, an effector protein does not require a tracrRNA to locate and/or cleave a target nucleic acid. In some embodiments, the crRNA of the guide nucleic acid comprises a repeat sequence and a spacer sequence, wherein the repeat sequence binds to the effector protein and the spacer sequence hybridizes to a target sequence of the target nucleic acid. The repeat sequence of the crRNA may interact with an effector protein, allowing for the guide nucleic acid and the effector protein to form a complex.

### III. Engineered Modifications

Polypeptides (e.g., effector proteins) and nucleic acids (e.g., engineered guide nucleic acids) can be further modified as described herein. Examples are modifications that do not alter the primary sequence of the polypeptides or nucleic acids, such as chemical derivatization of polypeptides (e.g., acylation, acetylation, carboxylation, amidation, etc.), or modifications that do alter the primary sequence of the polypeptide or nucleic acid. Also included are polypeptides that have a modified glycosylation pattern (e.g., those made by: modifying the glycosylation patterns of a polypeptide during its synthesis and processing or in further processing steps; by exposing the polypeptide to enzymes which affect glycosylation, such as mammalian glycosylating or deglycosylating enzymes). Also embraced are polypeptides that have phosphorylated amino acid residues or (e.g., phosphotyrosine, phosphoserine, phosphothreonine).

Modifications disclosed herein can also include modification of described polypeptides and/or guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable for their intended purpose (e.g., in vivo administration, in vitro methods, or ex vivo applications). Analogs of such polypeptides include those containing residues other than naturally occurring L-amino acids, e.g. D-amino acids or non-naturally occurring synthetic amino acids. D-amino acids may be substituted for some or all of the amino acid residues. Modifications can also include modifications with non-naturally occurring unnatural amino acids. The particular sequence and the manner of preparation will be determined by convenience, economics, purity required, and the like.

Modifications can further include the introduction of various groups to polypeptides and/or guide nucleic acids described herein. For example, groups can be introduced during synthesis or during expression of a polypeptide (e.g., an effector protein), which allow for linking to other molecules or to a surface. Thus, e.g., cysteines may be used to make thioethers, histidines for linking to a metal ion complex, carboxyl groups for forming amides or esters, amino groups for forming amides, and the like.

Modifications can further include changing of nucleic acids described herein (e.g., engineered guide nucleic acids) to provide the nucleic acid with a new or enhanced feature, such as improved stability. Such modifications of a nucleic acid include a base editing, a base modification, a backbone modification, a sugar modification, or combinations thereof. In some embodiments, the modifications can be of one or more nucleotides, nucleosides, or nucleobases in a nucleic acid.

In some embodiments, nucleic acids (e.g., nucleic acids encoding effector proteins, engineered guide nucleic acids, or nucleic acids encoding engineered guide nucleic acids) described herein comprise one or more modifications comprising: 2′O-methyl modified nucleotides, 2′ fluoro modified nucleotides; locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH₂ component parts; and combinations thereof.

### IV. Vectors and Multiplexed Expression Vectors

Compositions, systems, and methods described herein comprise a vector or a use thereof. A vector can comprise a nucleic acid of interest. In some embodiments, the nucleic acid of interest comprises one or more components of a composition or system described herein. In some embodiments, the nucleic acid of interest comprises a nucleotide sequence that encodes one or more components of the composition or system described herein. In some embodiments, one or more components comprises a polypeptide(s), guide nucleic acid(s), target nucleic acid(s), and donor nucleic acid(s). In some embodiments, the component comprises a nucleic acid encoding an effector protein, a donor nucleic acid, and a guide nucleic acid or a nucleic acid encoding the guide nucleic acid. The vector may be part of a vector system, wherein a vector system comprises a library of vectors each encoding one or more component of a composition or system described herein. In some embodiments, components described herein (e.g., an effector protein, a guide nucleic acid, and/or a target nucleic acid) are encoded by the same vector. In some embodiments, components described herein (e.g., an effector protein, a guide nucleic acid, and/or a target nucleic acid) are each encoded by different vectors of the system.

In some embodiments, a vector comprises a nucleotide sequence encoding one or more effector proteins as described herein. In some embodiments, the one or more effector proteins comprise at least two effector proteins. In some embodiments, the at least two effector protein are the same. In some embodiments, the at least two effector proteins are different from each other. In some embodiments, the nucleotide sequence is operably linked to a promoter that is operable in a target cell, such as a eukaryotic cell. In some embodiments, the vector comprises the nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more effector proteins.

In some embodiments, a vector may encode one or more of any system components, including but not limited to effector proteins, guide nucleic acids, donor nucleic acids, and target nucleic acids as described herein. In some embodiments, a system component encoding sequence is operably linked to a promoter that is operable in a target cell, such as a eukaryotic cell. In some embodiments, a vector may encode 1, 2, 3, 4 or more of any system components. For example, a vector may encode two or more guide nucleic acids, wherein each guide nucleic acid comprises a different sequence. A vector may encode an effector protein and a guide nucleic acid. A vector may encode an effector protein, a guide nucleic acid, and a donor nucleic acid.

In some embodiments, a vector comprises one or more guide nucleic acids, or a nucleotide sequence encoding the one or more guide nucleic acids as described herein. In some embodiments, the one or more guide nucleic acids comprise at least two guide nucleic acids. In some embodiments, the at least two guide nucleic acids are the same. In some embodiments, the at least two guide nucleic acids are different from each other. In some embodiments, the guide nucleic acid or the nucleotide sequence encoding the guide nucleic acid is operably linked to a promoter that is operable in a target cell, such as a eukaryotic cell. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids. In some embodiments, the vector comprises a nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids.

In some embodiments, a vector comprises one or more donor nucleic acids as described herein. In some embodiments, the one or more donor nucleic acids comprise at least two donor nucleic acids. In some embodiments, the at least two donor nucleic acids are the same. In some embodiments, the at least two donor nucleic acids are different from each other. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more donor nucleic acids.

In some embodiments, a vector may comprise or encode one or more regulatory elements. Regulatory elements may refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector may comprise or encode for one or more additional elements, such as, for example, replication origins, antibiotic resistance (or a nucleic acid encoding the same), a tag (or a nucleic acid encoding the same), selectable markers, and the like. In some embodiments, a vector comprises or encodes for one or more elements, such as, for example, ribosome binding sites, and RNA splice sites.

Vectors described herein can encode a promoter-a regulatory region on a nucleic acid, such as a DNA sequence, capable of initiating transcription of a downstream (3′ direction) coding or non-coding sequence. A promoter can be linked at its 3′ terminus to a nucleic acid, the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. The promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter.

Promotors may be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human Hl promoter (Hl). By transcriptional activation, it is intended that transcription will be increased above basal levels in the target cell by 2 fold, 5 fold, 10 fold, 50 fold, by 100 fold, 500 fold, or by 1000 fold, or more. In addition, vectors used for providing a nucleic acid that, when transcribed, produces a guide nucleic acid and/or a nucleic acid that encodes an effector protein to a cell may include nucleic acid sequences that encode for selectable markers in the target cells, so as to identify cells that have taken up the guide nucleic acid and/or the effector protein.

In general, vectors provided herein comprise at least one promotor or a combination of promoters driving expression or transcription of one or more genome editing tools described herein. In some embodiments, the vector comprises a nucleotide sequence of a promoter. In some embodiments, the vector comprises two promoters. In some embodiments, the vector comprises three promoters. In some embodiments, a length of the promoter is less than about 500, less than about 400, less than about 300, or less than about 200 linked nucleotides. In some embodiments, a length of the promoter is at least 100, at least 200, at least 300, at least 400, or at least 500 linked nucleotides. Non-limiting examples of promoters include CMV, 7SK, EF1a, RPBSA, hPGK, EFS, SV40, PGK1, Ubc, human beta actin, CAG, TRE, UAS, Ac5, Polyhedrin, CaMKIIa, GAL1-10, H1, TEF1, GDS, ADH1, CaMV35S, HSV TK, Ubi, U6, MNDU3, MSCV, MND and CAG.

In some embodiments, the promoter is a constitutive promoter. In some embodiments, the promoter is an inducible promoter. In some embodiments, the inducible promoter only drives expression of its corresponding coding sequence (e.g., polypeptide or guide nucleic acid) when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter. In some embodiments, the promoter for expressing effector protein is a ubiquitous promoter. In some embodiments, the ubiquitous promoter comprises MND or CAG promoter sequence.

In some embodiments, the promoters are prokaryotic promoters (e.g., drive expression of a gene in a prokaryotic cell). In some embodiments, the promoters are eukaryotic promoters, (e.g., drive expression of a gene in a eukaryotic cell). In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, a vector described herein is a nucleic acid expression vector. In some embodiments, a vector described herein is a recombinant expression vector. In some embodiments, a vector described herein is a messenger RNA.

In some embodiments, a vector described herein is a delivery vector. In some embodiments, the delivery vector is a eukaryotic vector, a prokaryotic vector (e.g., a bacterial vector) a viral vector, or any combination thereof. In some embodiments, the delivery vehicle is a non-viral vector. In some embodiments, the delivery vector is a plasmid. In some embodiments, the plasmid comprises DNA. In some embodiments, the plasmid comprises RNA. In some embodiments, the plasmid comprises circular double-stranded DNA. In some embodiments, the plasmid is linear. In some embodiments, the plasmid comprises one or more coding sequences of interest and one or more regulatory elements. In some embodiments, the plasmid comprises a bacterial backbone containing an origin of replication and an antibiotic resistance gene or other selectable marker for plasmid amplification in bacteria. In some embodiments, the plasmid is a minicircle plasmid. In some embodiments, the plasmid contains one or more genes that provide a selective marker to induce a target cell to retain the plasmid. In some examples, the plasmids are engineered through synthetic or other suitable means known in the art. For example, in some embodiments, the genetic elements are assembled by restriction digest of the desired genetic sequence from a donor plasmid or organism to produce ends of the DNA which is then be readily ligated to another genetic sequence.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R-U5′ segment in LTR of HTLV-I.

### Administration of a Non-Viral Vector

In some embodiments, an administration of a non-viral vector comprises contacting a cell, such as a host cell, with the non-viral vector. In some embodiments, a physical method or a chemical method is employed for delivering the vector into the cell. Exemplary physical methods include electroporation, gene gun, sonoporation, magnetofection, or hydrodynamic delivery. Exemplary chemical methods include delivery of the recombinant polynucleotide by liposomes such as, cationic lipids or neutral lipids; lipofection; dendrimers; lipid nanoparticle (LNP); or cell-penetrating peptides.

In some embodiments, a vector is administered as part of a method of nucleic acid detection, editing, and/or treatment as described herein. In some embodiments, a vector is administered in a single vehicle, such as a single expression vector. In some embodiments, at least two of the three components, a nucleic acid encoding one or more effector proteins, one or more donor nucleic acids, and one or more guide nucleic acids or a nucleic acid encoding the one or more guide nucleic acid, are provided in the single expression vector. In some embodiments, components, such as a guide nucleic acid and an effector protein, are encoded by the same vector. In some embodiments, an effector protein (or a nucleic acid encoding same) and/or an engineered guide nucleic acid (or a nucleic acid that, when transcribed, produces same) are not co-administered with donor nucleic acid in a single vehicle. In some embodiments, an effector protein (or a nucleic acid encoding same), an engineered guide nucleic acid (or a nucleic acid that, when transcribed, produces same), and/or donor nucleic acid are administered in one or more or two or more vehicles, such as one or more, or two or more expression vectors.

In some embodiments, a vector may be part of a vector system. In some embodiments, the vector system comprises a library of vectors each encoding one or more components of a composition or system described herein. In some embodiments, a vector system is administered as part of a method of nucleic acid detection, editing, and/or treatment as described herein, wherein at least two vectors are co-administered. In some embodiments, the at least two vectors comprise different components. In some embodiments, the at least two vectors comprise the same component having different sequences. In some embodiments, at least one of the three components, a nucleic acid encoding one or more effector proteins, one or more donor nucleic acids, and one or more guide nucleic acids or a nucleic acid encoding the one or more guide nucleic acids, or a variant thereof is provided in a different vector. In some embodiments, the nucleic acid encoding the effector protein, and a guide nucleic acid or a nucleic acid encoding the guide nucleic acid are provided in different vectors. In some embodiments, the donor nucleic acid is encoded by a different vector than the vector encoding the effector protein and the guide nucleic acid.

In some embodiments, the vector is a non-viral vector, and a physical method or a chemical method is employed for delivery into the somatic cell. Exemplary physical methods include electroporation, gene gun, sonoporation, magnetofection, or hydrodynamic delivery. Exemplary chemical methods include delivery of the recombinant polynucleotide via liposomes such as, cationic lipids or neutral lipids; dendrimers; nanoparticles; or cell-penetrating peptides.

In some embodiments, a fusion effector protein as described herein is inserted into a vector. In some embodiments, the vector optionally comprises one or more promoters, enhancers, ribosome binding sites, RNA splice sites, polyadenylation sites, a replication origin, and/or transcriptional terminator sequences.

In general, plasmids and vectors described herein comprise at least one promoter. In some embodiments, the promoters are constitutive promoters. In other embodiments, the promoters are inducible promoters. In additional embodiments, the promoters are prokaryotic promoters (e.g., drive expression of a gene in a prokaryotic cell). In some embodiments, the promoters are eukaryotic promoters, (e.g., drive expression of a gene in a eukaryotic cell). Exemplary promoters include, but are not limited to, CMV, EF1a, SV40, PGK1, Ubc, human beta actin, CAG, TRE, UAS, Ac5, polyhedron, CaMKIIa, GAL1-10, TEF1, GDS, ADH1, CaMV35S, Ubi, H1, U6, CaMV35S, SV40, CMV, and HSV TK promoter. In some embodiments, the promoter is CMV. In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R-U5′ segment in LTR of HTLV-I (Mol. Cell. Biol., Vol. 8 (1), p. 466-472, 1988); SV40 enhancer; the intron sequence between exons 2 and 3 of rabbit β-globin (Proc. Natl. Acad. Sci. USA., Vol. 78 (3), p. 1527-31, 1981); and the genome region of human growth hormone (J Immunol., Vol. 155 (3), p. 1286-95, 1995).

In some examples, the delivery vector may be a eukaryotic vector, a prokaryotic vector (e.g., a bacterial vector), a viral vector, or any combination thereof. In some embodiments, the delivery vehicle may be a non-viral vector. In some embodiments, the delivery vehicle may be a plasmid. In some embodiments, the plasmid comprises DNA. In some embodiments, the plasmid comprises RNA. In some examples, the plasmid comprises circular double-stranded DNA. In some examples, the plasmid may be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid may be a minicircle plasmid. In some examples, the plasmid contains one or more genes that provide a selective marker to induce a target cell to retain the plasmid. In some examples, the plasmid may be formulated for delivery through injection by a needle carrying syringe. In some examples, the plasmid may be formulated for delivery via electroporation. In some examples, the plasmids may be engineered through synthetic or other suitable means known in the art. For example, in some embodiments, the genetic elements may be assembled by restriction digest of the desired genetic sequence from a donor plasmid or organism to produce ends of the DNA which may then be readily ligated to another genetic sequence.

### Lipid Particles and Non-Viral Vectors

In some embodiments, compositions and systems provided herein comprise a lipid particle. In some embodiments, a lipid particle is a lipid nanoparticle (LNP). In some embodiments, a lipid or a lipid nanoparticle can encapsulate an expression vector as described herein. LNPs are a non-viral delivery system for delivery of the composition and/or system components described herein. LNPs are particularly effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28 (3): 146-157). In some embodiments, compositions and methods comprise a lipid, polymer, nanoparticle, or a combination thereof, or use thereof, to introduce one or more effector proteins, one or more guide nucleic acids, one or more donor nucleic acids, or any combinations thereof to a cell. Non-limiting examples of lipids and polymers are cationic polymers, cationic lipids, ionizable lipids, or bio-responsive polymers. In some embodiments, the ionizable lipids exploits chemical-physical properties of the endosomal environment (e.g., pH) offering improved delivery of nucleic acids. In some embodiments, the ionizable lipids are neutral at physiological pH. In some embodiments, the ionizable lipids are protonated under acidic pH. In some embodiments, the bio-responsive polymer exploits chemical-physical properties of the endosomal environment (e.g., pH) to preferentially release the genetic material in the intracellular space.

In some embodiments, a LNP comprises an outer shell and an inner core. In some embodiments, the outer shell comprises lipids. In some embodiments, the lipids comprise modified lipids. In some embodiments, the modified lipids comprise pegylated lipids. In some embodiments, the lipids comprise one or more of cationic lipids, anionic lipids, ionizable lipids, and non-ionic lipids. In some embodiments, the LNP comprises one or more of N1,N3,N5-tris(3-(didodecylamino)propyl)benzene-1,3,5-tricarboxamide (TT3), 2-dioleoyl-sn-glycero-3-phosphoethanolamine (DOPE), 1-palmitoyl-2-oleoylsn-glycero-3-phosphoethanolamine (POPE), 1,2-distearoyl-sn-glycero-3-phosphocholine (DSPC), cholesterol (Chol), 1,2-dimyristoyl-sn-glycerol, and methoxypolyethylene glycol (DMG-PEChooo), derivatives, analogs, or variants thereof. In some embodiments, the LNP has a negative net overall charge prior to complexation with one or more of a guide nucleic acid, a nucleic acid encoding the one or more guide nucleic acid, a nucleic acid encoding the effector protein, and/or a donor nucleic acid. In some embodiments, the inner core is a hydrophobic core. In some embodiments, the one or more of a guide nucleic acid, the nucleic acid encoding the one or more guide nucleic acid, the nucleic acid encoding the effector protein, and/or the donor nucleic acid forms a complex with one or more of the cationic lipids and the ionizable lipids. In some embodiments, the nucleic acid encoding the effector protein or the nucleic acid encoding the guide nucleic acid is self-replicating.

In some embodiments, a LNP comprises one or more of cationic lipids, ionizable lipids, and modified versions thereof. In some embodiments, the ionizable lipid comprises TT3 or a derivative thereof. Accordingly, in some embodiments, the LNP comprises one or more of TT3 and pegylated TT3. The publication WO2016187531 is hereby incorporated by reference in its entirety, which describes representative LNP formulations in Table 2 and Table 3, and representative methods of delivering LNP formulations in Example 7.

In some embodiments, a LNP comprises a lipid composition targeting to a specific organ. In some embodiments, the lipid composition comprises lipids having a specific alkyl chain length that controls accumulation of the LNP in the specific organ (e.g., liver or spleen). In some embodiments, the lipid composition comprises a biomimetic lipid that controls accumulation of the LNP in the specific organ (e.g., brain). In some embodiments, the lipid composition comprises lipid derivatives (e.g., cholesterol derivatives) that controls accumulation of the LNP in a specific cell (e.g., liver endothelial cells, Kupffer cells, hepatocytes).

### Delivery of Viral Vectors

In some embodiments, a vector described herein comprises a viral vector. In some embodiments, compositions, including pharmaceutical compositions, comprise a viral vector encoding a fusion effector protein and a guide nucleic acid, wherein at least a portion of the guide nucleic acid binds to the effector protein of the fusion effector protein. In some embodiments, pharmaceutical compositions comprise a viral vector encoding a fusion effector protein, an effector protein, a fusion partner, a guide nucleic acid, or a combination thereof, and a pharmaceutically acceptable excipient, carrier, or diluent.

A viral vector can be a nucleic acid to be delivered into a host cell via a recombinantly produced virus or viral particle. The nucleic acid may be single stranded, double stranded, linear or circular, segmented or non-segmented. The nucleic acid may comprise DNA, RNA, or a combination thereof. In some embodiments, the viral vector is a lentiviral vector. In some embodiments, the viral vector is a retroviral vector. A retroviral vector can also be referred to as a retrovirus. In some embodiments, the vector is an adeno-associated viral (AAV) vector. A viral vector delivered by such viruses or viral particles may be referred to by the type of virus to deliver the viral vector (e.g., an AAV viral vector is a viral vector that is to be delivered by an adeno-associated virus). A viral vector referred to by the type of virus to be delivered by the viral vector can contain viral elements (e.g., nucleotide sequences) necessary for packaging of the viral vector into the virus or viral particle, replicating the virus, or other desired viral activities. The virus may be a lentivirus. The virus may be an adenovirus. A virus containing a viral vector may be replication competent, replication deficient or replication defective. The virus may be a non-replicating virus. The virus may be an adeno-associated virus (AAV). The viral vector may be a retroviral vector. Retroviral vectors may include gamma-retroviral vectors such as vectors derived from the Moloney Murine Keukemia Virus (MoMLV, MMLV, MuLV, or MLV) or the Murine Steam cell Virus (MSCV) genome. Retroviral vectors may include lentiviral vectors such as those derived from the human immunodeficiency virus (HIV) genome. In some embodiments, the viral vector is a chimeric viral vector, comprising viral portions from two or more viruses. In some embodiments, the viral vector is a recombinant viral vector.

In some embodiments, the viral vector is an AAV. The AAV may be any AAV known in the art. In some embodiments, the viral vector corresponds to a virus of a specific serotype. In some examples, the serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, an AAV12 serotype, AAV-rh10 serotype, and any combination, derivative, or variant thereof. In some embodiments the AAV vector is a recombinant vector, a hybrid AAV vector, a chimeric AAV vector, a self-complementary AAV (scAAV) vector, a single-stranded AAV or any combination thereof. scAAV genomes are generally known in the art and contain both DNA strands which can anneal together to form double-stranded DNA.

In some embodiments, the AAV vector may be a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some embodiments, an AAV vector described herein is a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some embodiments, AAV vector described herein comprises two inverted terminal repeats (ITRs). According, in some embodiments, the viral vector provided herein comprises two inverted terminal repeats of AAV. A nucleotide sequence between the ITRs of an AAV vector provided herein comprises a sequence encoding genome editing tools. In some embodiments, the genome editing tools comprise a nucleic acid encoding one or more effector proteins, a nucleic acid encoding one or more fusion proteins (e.g., a nuclear localization signal (NLS), polyA tail), one or more guide nucleic acids, a nucleic acid encoding the one or more guide nucleic acids, respective promoter(s), one or more donor nucleic acid, or any combinations thereof. In some embodiments, viral vectors provided herein comprise at least one promotor or a combination of promoters driving expression or transcription of one or more genome editing tools described herein. In some embodiments, a coding region of the AAV vector forms an intramolecular double-stranded DNA template thereby generating the AAV vector that is a self-complementary AAV (scAAV) vector. In some embodiments, the scAAV vector comprises the sequence encoding genome editing tools that has a length of about 2 kb to about 3 kb. In some embodiments, the AAV vector provided herein is a self-inactivating AAV vector. In some embodiments, the AAV vector provided herein comprises a modification, such as an insertion, deletion, chemical alteration, or synthetic modification, relative to a wild-type AAV vector.

### Producing AAV Delivery Vectors

In some embodiments, methods of producing delivery vectors herein comprise packaging a nucleic acid encoding an effector protein and a guide nucleic acid, or a combination thereof, into an AAV vector. In some embodiments, methods of producing the delivery vector comprises, (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging a Cas effector encoding nucleic acid into the AAV particle, thereby generating an AAV delivery vector. In some embodiments, promoters, stuffer sequences, and any combination thereof may be packaged in the AAV vector. In some examples, the AAV vector can package 1, 2, 3, 4, or 5 guide nucleic acids or copies thereof. In some embodiments, the AAV vector comprises inverted terminal repeats, e.g., a 5′ inverted terminal repeat and a 3′ inverted terminal repeat. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

### V. Target Nucleic Acids

Disclosed herein are compositions, systems, devices, kits and methods for modifying and detecting target nucleic acids. In some embodiments, the target nucleic acid is a double stranded nucleic acid. In some embodiments, the target nucleic acid is a single stranded nucleic acid. In some embodiments, the target nucleic acid is a double stranded nucleic acid that is prepared into single stranded nucleic acids before or upon contacting a reagent or sample. In some embodiments, the target nucleic acid comprises DNA. In some embodiments, the target nucleic acid comprises RNA. The target nucleic acids include but are not limited to mRNA, rRNA, tRNA, non-coding RNA, long non-coding RNA, and microRNA (miRNA). In some embodiments, the target nucleic acid is complementary DNA (cDNA) synthesized from a single-stranded RNA template in a reaction catalyzed by a reverse transcriptase. In some embodiments, the target nucleic acid is single-stranded RNA (ssRNA) or mRNA. In some embodiments, a target nucleic acid can comprise a combination of RNA and DNA.

In some embodiments, a target nucleic acid comprising a target sequence comprises a PAM sequence. In some embodiments, the PAM sequence is 3′ to the target sequence. In some embodiments, the PAM sequence is directly 3′ to the target sequence. In some embodiments, the PAM sequence 5′ to the target sequence. In some embodiments, the PAM sequence is directly 5′ to the target sequence. In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by an effector protein system.

In some embodiments, a target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleotides. In some embodiments, the target nucleic acid comprises 10 to 90, 20 to 80, 30 to 70, or 40 to 60 linked nucleotides. In some embodiments, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleotides. In some embodiments, the target nucleic acid comprises at least 10, at least 20, at least 30, at least 40, at least 50, at least 60, at least 70, at least 80, at least 90, or at least 100 linked nucleotides.

In some embodiments, compositions, systems, devices, kits and methods described herein comprise a target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides). In some embodiments, the target nucleic acid has undergone a modification (e.g., an editing) after contacting with an RNP. In some embodiments, the editing is a change in the sequence of the target nucleic acid. In some embodiments, the change comprises an insertion, deletion, or substitution of one or more nucleotides compared to the target nucleic acid that has not undergone any modification.

In some embodiments, the target nucleic acid comprises a nucleic acid sequence from a pathogen responsible for a disease. Non-limiting examples of pathogens are bacteria, a virus and a fungus. The target nucleic acid, in some embodiments, is a portion of a nucleic acid from a sexually transmitted infection or a contagious disease. In some embodiments, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), Chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. A pathogenic virus can be a DNA virus or an RNA virus. Pathogenic viruses include but are not limited to coronavirus (e.g., SARS-CoV-2); immunodeficiency virus (e.g., HIV); Orthopoxvirus (e.g., monkeypox virus, cowpox virus, camelpox virus, horsepox virus, vaccinia virus, and variola virus); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some embodiments, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

In some embodiments, the target nucleic acid comprises a nucleic acid sequence of a virus, a bacterium, or other pathogen responsible for a disease in a plant (e.g., a crop). Methods and compositions of the disclosure may be used to treat or detect a disease in a plant. For example, the methods of the disclosure may be used to target a viral nucleic acid sequence in a plant. An effector protein of the disclosure may cleave the viral nucleic acid. In some embodiments, the target nucleic acid comprises a nucleic acid sequence of a virus or a bacterium or other agents (e.g., any pathogen) responsible for a disease in the plant (e.g., a crop). In some embodiments, the target nucleic acid comprises RNA. The target nucleic acid, in some embodiments, is a portion of a nucleic acid from a virus or a bacterium or other agents responsible for a disease in the plant (e.g., a crop). In some embodiments, the target nucleic acid is a portion of a nucleic acid from a genomic locus, or any NA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant may be an RNA virus. A virus infecting the plant may be a DNA virus. Non-limiting examples of viruses that may be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

In some embodiments, a target nucleic acid comprises a portion or a specific region of a nucleic acid from a genomic locus, any DNA amplicon of, a reverse transcribed mRNA, or a cDNA from a gene described herein. In some embodiments, the target nucleic acid is an amplicon of at least a portion of a gene. Non-limiting examples of genes are recited in TABLE 7. Nucleic acid sequences of target nucleic acids and/or corresponding genes are readily available in public databases as known and used in the art. In some embodiments, the target nucleic acid is selected from TABLE 7. In some embodiments, the target nucleic acid comprises one or more target sequences. In some embodiments, the one or more target sequence is within any one of the target nucleic acids set forth in TABLE 7.

Nucleic acids, such as DNA and pre-mRNA, described herein can contain at least one intron and at least one exon, wherein as read in the 5′ to the 3′ direction of a nucleic acid strand, the 3′ end of an intron can be adjacent to the 5′ end of an exon, and wherein said intron and exon correspond for transcription purposes. If a nucleic acid strand contains more than one intron and exon, the 5′ end of the second intron is adjacent to the 3′ end of the first exon, and 5′ end of the second exon is adjacent to the 3′ end of the second intron. The junction between an intron and an exon can be referred to herein as a splice junction, wherein a 5′ splice site (SS) can refer to the +1/+2 position at the 5′ end of intron and a 3′SS can refer to the last two positions at the 3′ end of an intron. Alternatively, a 5′ SS can refer to the 5′ end of an exon and a 3′SS can refer to the 3′ end of an exon. In some embodiments, nucleic acids can contain one or more elements that act as a signal during transcription, splicing, and/or translation. In some embodiments, signaling elements include a 5′SS, a 3′SS, a premature stop codon, U1 and/or U2 binding sequences, and cis acting elements such as branch site (BS), polypyridine tract (PYT), exonic and intronic splicing enhancers (ESEs and ISEs) or silencers (ESSs and ISSs). In some embodiments, nucleic acids may also comprise a untranslated region (UTR), such as a 5′ UTR or a 3′ UTR. In some embodiments, the start of an exon or intron is referred to interchangeably herein as the 5′ end of an exon or intron, respectively. Likewise, in some embodiments, the end of an exon or intron is referred to interchangeably herein as the 3′ end of an exon or intron, respectively.

In some embodiments, at least a portion of at least one target sequence is within about 1, about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 to about 300 nucleotides adjacent to: the 5′ end of an exon; the 3′ end of an exon; the 5′ end of an intron; the 3′ end of an intron; one or more signaling element comprising a 5′SS, a 3′SS, a premature stop codon, U1 binding sequence, U2 binding sequence, a BS, a PYT, ESE, an ISE, an ESS, an ISS; a 5′ UTR; a 3′ UTR; more than one of the foregoing, or any combination thereof. In some embodiments, the target nucleic acid comprises a target locus. In some embodiments, the target nucleic acid comprises more than one target loci. In some embodiments, the target nucleic acid comprises two target loci. Accordingly, in some embodiments, the target nucleic acid can comprise one or more target sequences.

In some embodiments, compositions, systems, and methods described herein comprise an edited target nucleic acid which can describe a target nucleic acid wherein the target nucleic acid has undergone a change, for example, after contact with an effector protein. In some embodiments, the editing is an alteration in the sequence of the target nucleic acid. In some embodiments, the edited target nucleic acid comprises an insertion, deletion, or replacement of one or more nucleotides compared to the unedited target nucleic acid. In some embodiments, the editing is a mutation.

In some embodiments, the target nucleic acid is any one of: a naturally occurring eukaryotic sequence, a naturally occurring prokaryotic sequence, a naturally occurring viral sequence, a naturally occurring bacterial sequence, a naturally occurring fungal sequence, an engineered eukaryotic sequence, an engineered prokaryotic sequence, an engineered viral sequence, an engineered bacterial sequence, an engineered fungal sequence, a fragment of a naturally occurring sequence, a fragment of an engineered sequence, and combinations thereof.

In some embodiments, the target nucleic acid is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell.

### Mutations

In some embodiments, target nucleic acids comprise a mutation. In some embodiments, a composition, system, devices, kits or method described herein can be used to modify a target nucleic acid comprising a mutation such that the mutation is modified to be a wild-type nucleotide or nucleotide sequence. In some embodiments, a composition, system, devices, kits or method described herein can be used to detect a target nucleic acid comprising a mutation. The mutation may be a mutation of 1, 2, 3, 4, 5, 6, 7, 8, 9, 10, 11, 12, 13, 14, 15, 16, 17, 18, 19, 20 or more nucleotides. The mutation may be in an open reading frame of a target nucleic acid. The mutation may result in the insertion of at least one amino acid in a polypeptide encoded by the target nucleic acid. The mutation may result in the deletion of at least one amino acid in a polypeptide encoded by the target nucleic acid. The mutation may result in the substitution of at least one amino acid in a polypeptide encoded by the target nucleic acid. The mutation may result in misfolding of the polypeptide. A mutation that results in the deletion, insertion, or substitution of one or more amino acids of a protein encoded by the target nucleic acid may result in misfolding of a protein encoded by the target nucleic acid. The mutation may result in a premature stop codon. The mutation may result in a truncation of the protein. A mutation may result in a premature stop codon, thereby resulting in a truncation of the encoded protein.

In some embodiments, mutations comprise an insertion-deletion (indel), a point mutation, a chromosomal mutation, a copy number mutation, a single nucleotide polymorphism (SNP), a variation, a frameshift mutation or any combination thereof. In some embodiments, an indel mutation is an insertion or deletion of one or more nucleotides. A point mutation may be a substitution, insertion, or deletion of a single nucleotide. In some embodiments, mutations comprise a chromosomal mutation. A chromosomal mutation may comprise an inversion, a deletion, a duplication, or a translocation of one or more nucleotides. In some embodiments, mutations comprise a copy number variation. A copy number variation may comprise a gene amplification or an expanding trinucleotide repeat. In some embodiments, a frameshift mutation occurs when the number of nucleotides in the insertion/deletion is not divisible by three, and it occurs in a protein coding region. In some embodiments, guide nucleic acids described herein hybridize to a target sequence of a target nucleic acid comprising the mutation. In some embodiments, mutations are located in a non-coding region of a gene.

In some embodiments, at least a portion of a guide nucleic acid of a composition, device, kit, method, or system described herein hybridizes to a region of the target nucleic acid comprising the mutation. In some embodiments, at least a portion of a guide nucleic acid of a composition, device, kit, method or system described herein hybridizes to a region of the target nucleic acid that is within 10 nucleotides, within 50 nucleotides, within 100 nucleotides, or within 200 nucleotides of the mutation. The mutation may be located in a non-coding region or a coding region of a gene. The mutation may be located in a non-coding region or a coding region of a gene, wherein the gene is a target nucleic acid. A mutation may be in an open reading frame of a target nucleic acid. In some embodiments, guide nucleic acids described herein hybridize to a portion of the target nucleic acid comprising or adjacent to the mutation.

In some embodiments, the mutation is an autosomal dominant mutation. In some embodiments, the mutation is a dominant negative mutation. In some embodiments, the mutation is a loss of function mutation. In some embodiments, the mutation is a single nucleotide polymorphism (SNP). In some embodiments, the SNP is associated with a phenotype of the sample or a phenotype of the organism from which the sample was taken. The SNP, in some embodiments, is associated with altered phenotype from wild type phenotype. The SNP may be a synonymous substitution or a nonsynonymous substitution. The nonsynonymous substitution may be a missense substitution, or a nonsense point mutation. The synonymous substitution may be a silent substitution. The mutation may be a deletion of one or more nucleotides. Often, the single nucleotide mutation, SNP, or deletion is associated with a disease such as cancer or a genetic disorder. The mutation, such as a single nucleotide mutation, a SNP, or a deletion, may be encoded in the sequence of a target nucleic acid from the germline of an organism or may be encoded in a target nucleic acid from a diseased cell, such as a cancer cell.

In some embodiments, the target nucleic acid comprises a mutation associated with a disease. In some examples, a mutation associated with a disease refers to a mutation whose presence in a subject indicates that the subject is susceptible to, or suffers from, a disease, disorder, or pathological state. In some examples, a mutation associated with a disease refers to a mutation which causes the disease, contributes to the development of the disease, or indicates the existence of the disease. A mutation associated with a disease may also refer to any mutation which generates transcription or translation products at an abnormal level, or in an abnormal form, in cells affected by a disease relative to a control without the disease. In some embodiments, the mutation causes the disease.

Non-limiting examples of diseases associated with genetic mutations are cystic fibrosis, Duchenne muscular dystrophy, β-thalassemia, hemophilia, sickle cell anemia, amyotrophic lateral sclerosis (ALS), severe combined immunodeficiency, Huntington's disease, Alzheimer's Disease, alpha-1 antitrypsin deficiency, myotonic dystrophy Type 1, and Usher syndrome. The disease may comprise, at least in part, a cancer, an inherited disorder, an ophthalmological disorder, a neurological disorder, a blood disorder, a metabolic disorder, or a combination thereof.

In some embodiments, a target nucleic acid comprises a mutation of 1, 2, 3, 4, 5, 6, 7, 8, 9, 10, 11, 12, 13, 14, 15, 16, 17, 18, 19, 20 or more nucleotides. The mutation may comprise a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may comprise a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides.

In some embodiments, target nucleic acids comprise a mutation, wherein the mutation is a SNP. In some embodiments, the single nucleotide mutation or SNP is associated with a phenotype of the sample or a phenotype of the organism from which the sample was taken. In some embodiments, the SNP is associated with altered phenotype from wild type phenotype. In some embodiments, a single nucleotide mutation, SNP, or deletion described herein is associated with a disease, such as a genetic disease. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution. In some embodiments, the mutation is a deletion of one or more nucleotides. In some embodiments, the single nucleotide mutation, SNP, or deletion is associated with a disease such as a genetic disorder. In some embodiments, the mutation, such as a single nucleotide mutation, a SNP, or a deletion, may be encoded in the sequence of a target nucleic acid from the germline of an organism or may be encoded in a target nucleic acid from a diseased cell.

In some embodiments, the mutation is associated with a disease, such as a genetic disorder. In some embodiments, the mutation may be encoded in the sequence of a target nucleic acid from the germline of an organism or may be encoded in a target nucleic acid from a diseased cell. In some embodiments, a target nucleic acid described herein comprises a mutation associated with a disease. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. A mutation associated with a disease may also refer to any mutation which generates transcription or translation products at an abnormal level, or in an abnormal form, in cells affected by a disease relative to a control without the disease. In some examples, a mutation associated with a disease refers to a mutation whose presence in a subject indicates that the subject is susceptible to, or suffers from, a disease, disorder, or pathological state. In some embodiments, a mutation associated with a disease, comprises the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation. In some embodiments, a target nucleic acid described herein comprises a mutation associated with a disease, wherein the target nucleic acid is any one of the target nucleic acids set forth in TABLE 7. In some embodiments, a target nucleic acid described herein comprises a mutation associated with a disease, wherein the disease is any one of the diseases set forth in TABLE 8.

### Certain Target Nucleic Acids

The target nucleic acid, in some embodiments, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker, such as a prostate cancer biomarker or non-small cell lung cancer. In some embodiments, the assay may be used to detect “hotspots” in target nucleic acids that may be predictive of lung cancer. In some embodiments, the target nucleic acid comprises a portion of a nucleic acid that is associated with a blood fever.

Described herein are compositions, devices, kits, systems and methods for editing or detecting a target nucleic acid, wherein the target nucleic acid is a gene, a portion thereof, a transcript thereof. In some embodiments, the target nucleic acid is a reverse transcript (e.g., a cDNA) of an mRNA transcribed from the gene, or an amplicon thereof. In some embodiments, the target nucleic acid is an amplicon of at least a portion of a gene. Non-limiting examples of target nucleic acids (e.g., genes) are as set forth in TABLE 7. Any region of the aforementioned gene loci may be probed for a mutation or deletion using the compositions, devices, kits, systems and methods disclosed herein. For example, in the EGFR gene locus, the compositions, devices, kits, systems and methods for detection disclosed herein may be used to detect a single nucleotide polymorphism or a deletion.

In some embodiments, a target nucleic acid is in a cell. In some embodiments, the cell is a single-cell eukaryotic organism; a plant cell an algal cell; a fungal cell; an animal cell; a cell of an invertebrate animal; a cell of a vertebrate animal such as fish, amphibian, reptile, bird, and mammal; or a cell of a mammal such as a human, a non-human primate, an ungulate, a feline, a bovine, an ovine, and a caprine. In some embodiments, the cell is a eukaryotic cell. In some embodiments, the cell is a mammalian cell, a human cell, or a plant cell. In some embodiments, the cell is a human cell. In some embodiments, the human cell is a: muscle cell, liver cell, lung cell, cardiac cell, visceral cell, cardiac muscle cell, smooth muscle cell, cardiomyocyte, nodal cardiac muscle cell, smooth muscle cell, visceral muscle cell, skeletal muscle cell, myocyte, red (or slow) skeletal muscle cell, white (fast) skeletal muscle cell, intermediate skeletal muscle, muscle satellite cell, muscle stem cell, myoblast, muscle progenitor cell, induced pluripotent stem cell (iPS), or a cell derived from an iPS cell, modified to have its gene edited and differentiated into myoblasts, muscle progenitor cells, muscle satellite cells, muscle stem cells, skeletal muscle cells, cardiac muscle cells or smooth muscle cells.

### VI. Compositions

Disclosed herein are compositions comprising one or more effector proteins described herein or nucleic acids encoding the one or more effector proteins, one or more guide nucleic acids described herein or nucleic acids encoding the one or more guide nucleic acids described herein, or combinations thereof. In some embodiments, one or more of a repeat sequence, a crRNA, or both of the one or more guide nucleic acids are capable of interacting with the one or more of the effector proteins. In some embodiments, spacer sequences of the one or more guide nucleic acids hybridizes with a target sequence of a target nucleic acid. In some embodiments, the compositions comprise one or more donor nucleic acids described herein. In some embodiments, the compositions are capable of editing a target nucleic acid in a cell or a subject. In some embodiments, the compositions are capable of editing a target nucleic acid or the expression thereof in a cell, in a tissue, in an organ, in vitro, in vivo, or ex vivo. In some embodiments, the compositions are capable of editing a target nucleic acid in a sample comprising the target nucleic.

In some embodiments, compositions described herein comprise plasmids described herein, viral vectors described herein, non-viral vectors described herein, or combinations thereof. In some embodiments, compositions described herein comprise the viral vectors. In some embodiments, compositions described herein comprise an AAV. In some embodiments, compositions described herein comprise liposomes (e.g., cationic lipids or neutral lipids), dendrimers, lipid nanoparticle (LNP), or cell-penetrating peptides. In some embodiments, compositions described herein comprise an LNP.

Disclosed herein, in some embodiments, are compositions comprising an effector protein and a guide nucleic acid, wherein the effector protein comprises an amino acid sequence that is at least 75%, at least 80%, at least 85%, at least 90%, at least 95%, at least 98%, at least 99% or 100% identical to any one of SEQ ID NOs: 1-28 and 93-142. Also disclosed herein, in some embodiments, are compositions comprising an effector protein and a guide nucleic acid, wherein the amino acid sequence of the effector protein is at least 75%, at least 80%, at least 85%, at least 90%, at least 95%, at least 98%, at least 99% or 100% identical to any one of SEQ ID NOs: 1-28 and 93-142. Also disclosed herein, in some embodiments, are compositions comprising an effector protein and a guide nucleic acid, wherein the effector protein comprises about 100, about 120, about 140, about 160, about 180, about 200, about 220, about 240, about 260, about 280, about 300, about 320, about 340, about 360, about 380, about 400, about 420, about 440, about 460, about 480, about 500, about 520, about 540, about 560, about 580, about 600, about 620, about 640, about 660, about 680, about 700, about 720, about 740, about 760, about 780, about 800, about 820, about 840, about 860, about 880, about 900, about 920, about 940, about 960, about 980, about 1000, about 1020, about 1040, about 1060, about 1080, about 1100, about 1120, about 1140, about 1160, about 1180, about 1200, about 1220, about 1240, about 1260, about 1280, about 1300, about 1320, about 1340, or about 1360 contiguous amino acids of a sequence selected from any one of SEQ ID NOS: 1-28 and 93-142. Also disclosed herein, in some embodiments, are compositions comprising an effector protein and a guide nucleic acid, wherein the effector protein comprises the amino acid sequence located at positions 1-100, 150-250, 101-200, 250-350, 201-300, 350-450, 301-400, 350-450, 401-500, 450-550, 501-600, 550-650, 601-700, 650-750, 701-800, 750-850, 801-900, 850-950, 901-1000, 950-1050, 1001-1100, 1050-1150, 1101-1200, 1150-1250, 1201-1300, or 1250-1350 of a sequence selected from any one of SEQ ID NOS: 1-28 and 93-142. Also disclosed herein, in some embodiments, are compositions comprising an effector protein and a guide nucleic acid, wherein the effector protein comprises an amino acid sequence that is at least 90%, at least 95%, or 100% identical to a portion of a sequence selected from any one of SEQ ID NOS: 1-28 and 93-142, wherein the portion of the sequence is about 30%, about 40% about 50%, about 60%, about 70%, about 80%, or about 90% of a sequence selected from any one of SEQ ID NOS: 1-28 and 93-142. In some embodiments, at least a portion of the guide nucleic acid binds the effector protein. In some embodiments, the guide nucleic acid comprises a crRNA. In some embodiments, the guide nucleic acid comprises a tracrRNA sequence. In some embodiments, the guide nucleic acid does not comprise a tracrRNA. In some embodiments, the guide nucleic acid comprises a crRNA and a tracrRNA sequence. In some embodiments, the guide nucleic acid comprises a first sequence and a second sequence, wherein the first sequence is heterologous with the second sequence. In some embodiments, the first sequence comprises at least five nucleotides and the second sequence comprises at least five nucleotides. In some embodiments, the effector protein comprises a nuclear localization signal. In some embodiments, the length of the effector protein is at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, or at least 1300 linked amino acid residues. In some embodiments, the length of the effector protein is less than about 1900 linked amino acids. In some embodiments, the length of the effector protein is about 300 to about 400, about 350 to about 450, about 400 to about 500, about 450 to about 550, about 500 to about 600, about 550 to about 650, about 600 to about 700, about 650 to about 750, about 700 to about 800, about 750 to about 850, about 800 to about 900, about 850 to about 950, about 900 to about 1000, about 950 to about 1050, about 1000 to about 1100, about 1050 to about 1150, about 1100 to about 1200, about 1150 to about 1250, about 1200 to about 1300, or about 1250 to about 1350 linked amino acids. In some embodiments, compositions comprise a donor nucleic acid. In some embodiments, compositions comprise a fusion partner protein linked to the effector protein. In some embodiments, the fusion partner protein is directly fused to the N terminus or C terminus of the effector protein via an amide bond. In some embodiments, the fusion partner protein is directly fused to the N terminus or C terminus of the effector protein via a peptide linker. In some embodiments, the fusion partner protein comprises a polypeptide selected from a deaminase, a transcriptional activator, a transcriptional repressor, or a functional domain thereof. In some embodiments, the effector protein comprises at least one mutation that reduces its nuclease activity relative to the effector protein without the mutation as measured in a cleavage assay. In some embodiments, the effector protein is a catalytically inactive nuclease. In some embodiments, the effector protein and the guide nucleic acid do not occur together in nature.

Disclosed herein, in some embodiments are compositions comprising a nucleic acid expression vector encoding an effector protein, wherein the effector protein comprises an amino acid sequence that is at least 75%, at least 80%, at least 85%, at least 90%, at least 95%, at least 98%, at least 99% or 100% identical to any one of SEQ ID NOs: 1-28, or in SEQ ID NOs: 93-142. Also disclosed herein, in some embodiments, are compositions comprising a nucleic acid expression vector encoding an effector protein, wherein the amino acid sequence of the effector protein is at least 75%, at least 80%, at least 85%, at least 90%, at least 95%, at least 98%, at least 99% or 100% identical to any one of SEQ ID NOs: 1-28, or in SEQ ID NOs: 93-142. Also disclosed herein, in some embodiments, are compositions comprising a nucleic acid expression vector encoding an effector protein, wherein the effector protein comprises about 100, about 120, about 140, about 160, about 180, about 200, about 220, about 240, about 260, about 280, about 300, about 320, about 340, about 360, about 380, about 400, about 420, about 440, about 460, about 480, about 500, about 520, about 540, about 560, about 580, about 600, about 620, about 640, about 660, about 680, about 700, about 720, about 740, about 760, about 780, about 800, about 820, about 840, about 860, about 880, about 900, about 920, about 940, about 960, about 980, about 1000, about 1020, about 1040, about 1060, about 1080, about 1100, about 1120, about 1140, about 1160, about 1180, about 1200, about 1220, about 1240, about 1260, about 1280, about 1300, about 1320, or about 1340 contiguous amino acids of a sequence selected from any one of SEQ ID NOS: 1-28 and 93-142. Also disclosed herein, in some embodiments, are compositions comprising a nucleic acid expression vector encoding an effector protein, wherein the effector protein comprises the amino acid sequence located at positions 1-100, 150-250, 101-200, 250-350, 201-300, 350-450, 301-400, 350-450, 401-500, 450-550, 501-600, 550-650, 601-700, 650-750, 701-800, 750-850, 801-900, 850-950, 901-1000, 950-1050, 1001-1100, 1050-1150, 1101-1200, 1150-1250, 1201-1300, or 1250-1350 of a sequence selected from any one of SEQ ID NOS: 1-28 and 93-142. Also disclosed herein, in some embodiments, are compositions comprising a nucleic acid expression vector encoding an effector protein, wherein the effector protein comprises an amino acid sequence that is at least 90%, at least 95%, or 100% identical to a portion of a sequence selected from any one of SEQ ID NOS: 1-28 and 93-142, wherein the portion of the sequence is about 30%, about 40% about 50%, about 60%, about 70%, about 80%, or about 90% of a sequence selected from any one of SEQ ID NOS: 1-28 and 93-142. In some embodiments, the nucleic acid expression vector encodes at least one guide nucleic acid. In some embodiments, compositions comprise an additional nucleic acid expression vector encoding an engineered guide nucleic acid. In some embodiments, compositions comprise a donor nucleic acid, optionally wherein the donor nucleic acid is encoded by the nucleic acid expression vector or additional nucleic acid expression vector. In some embodiments, the nucleic acid expression vector is contained within a viral vector. In some embodiments, the viral vector is an adeno associated viral (AAV) vector.

Disclosed herein, in some embodiments, are compositions comprising a virus, wherein the virus comprises a nucleic acid expression vector encoding an effector protein, wherein the effector protein comprises an amino acid sequence that is at least 75%, at least 80%, at least 85%, at least 90%, at least 95%, at least 98%, at least 99% or 100% identical to any one of SEQ ID NOs: 1-28 and 93-142. In some embodiments, the composition comprises a salt, such as potassium acetate. In some embodiments, the concentration of the salt in the composition is 0.001 mM to 200 mM. In some embodiments, the effector protein comprises or consists of an amino acid sequence that is at least 75%, at least 80%, at least 85%, at least 90%, at least 95%, at least 98%, at least 99% or 100% identical to any one of SEQ ID NOs: 1, 3, 4, 7, 9, 18, 19, 20, 21, 23, 24, 25, and 26, and the concentration of the salt in the composition is about 100 mM to about 200 mM.

### Pharmaceutical Compositions and Modes of Administration

Disclosed herein, in some aspects, are pharmaceutical compositions for modifying a target nucleic acid in a cell or a subject, comprising any one of the effector proteins, engineered effector proteins, or fusion effector proteins described herein. Also disclosed herein, in some aspects, are pharmaceutical compositions comprising a nucleic acid encoding any one of the effector proteins, engineered effector proteins, or fusion effector proteins described herein. In some embodiments, pharmaceutical compositions comprise a guide nucleic acid. In some embodiments, pharmaceutical compositions comprise a plurality of guide nucleic acids. Pharmaceutical compositions may be used to modify a target nucleic acid or the expression thereof in a cell in vitro, in vivo or ex vivo.

In some embodiments, pharmaceutical compositions comprise one or more nucleic acids encoding an effector protein, fusion effector protein, fusion partner, a guide nucleic acid, or a combination thereof, and a pharmaceutically acceptable excipient, carrier, or diluent. The effector protein, fusion effector protein, fusion partner protein, or combination thereof may be any one of those described herein. The one or more nucleic acids may comprise a plasmid. The one or more nucleic acids may comprise a nucleic acid expression vector. A nucleic acid expression vector can refer to a plasmid that can be used to express a nucleic acid of interest. The one or more nucleic acids may be contained within a viral vector.

Also disclosed herein, in some embodiments, are pharmaceutical compositions comprising: an effector protein or a nucleic acid expression vector encoding the effector protein, wherein the effector protein comprises an amino acid sequence that is at least 75%, at least 80%, at least 85%, at least 90%, at least 95%, at least 98%, at least 99% or 100% identical to any one of SEQ ID NOs: 1-28 and 93-142; and a pharmaceutically acceptable excipient, carrier or diluent.

Pharmaceutical compositions described herein may comprise a salt. In some embodiments, the salt is a sodium salt. In some embodiments, the salt is a potassium salt. In some embodiments, the salt is a magnesium salt. In some embodiments, the salt is sodium chloride. In some embodiments, the salt is potassium nitrate. In some embodiments, the salt is Mg²⁺SO₄²⁻.

Non-limiting examples of pharmaceutically acceptable excipients, carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethyleneglycol; and preservatives.

In some embodiments, pharmaceutical compositions are in the form of a solution (e.g., a liquid). The solution may be formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH of the solution is less than 7. In some embodiments, the pH is greater than 7.

VII. Methods and Formulations for Introducing System Components and Compositions into a Target Cell

A guide nucleic acid (or a nucleic acid comprising a nucleotide sequence encoding same) and/or an effector protein described herein may be introduced into a host cell by any of a variety of well-known methods. As a non-limiting example, a guide nucleic acid and/or effector protein may be combined with a lipid. As another non-limiting example, a guide nucleic acid and/or effector protein may be combined with a particle or formulated into a particle.

### Methods for Introducing System Components and Compositions to a Host

Described herein are methods of introducing various components described herein to a host. A host may be any suitable host, such as a host cell. When described herein, a host cell may be an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells may be, or have been, used as recipients for methods of introduction described herein, and include the progeny of the original cell which has been transformed by the methods of introduction described herein. It is understood that the progeny of a single cell may not necessarily be completely identical in morphology or in genomic or total DNA complement as the original parent, due to natural, accidental, or deliberate mutation. A host cell may be a recombinant host cell or a genetically modified host cell, if a heterologous nucleic acid, e.g., an expression vector, has been introduced into the cell.

Methods of introducing a nucleic acid and/or protein into a host cell are known in the art, and any convenient method may be used to introduce a subject nucleic acid (e.g., an expression construct/vector) into a target cell (e.g., a human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: S0169-409X(12)00283-9. doi: 10.1016/j.addr.2012.09.023), and the like. In some embodiments, the nucleic acid and/or protein are introduced into a disease cell comprised in a pharmaceutical composition comprising the guide nucleic acid and/or effector protein and a pharmaceutically acceptable excipient.

In some embodiments, molecules of interest, such as nucleic acids of interest, are introduced to a host. In some embodiments, polypeptides, such as an effector protein are introduced to a host. In some embodiments, vectors, such as lipid particles and/or viral vectors may be introduced to a host. Introduction may be for contact with a host or for assimilation into the host, for example, introduction into a host cell.

In some embodiments, described herein are methods of introducing one or more nucleic acids, such as a nucleic acid encoding an effector protein, a nucleic acid that, when transcribed, produces an engineered guide nucleic acid, and/or a donor nucleic acid, or combinations thereof, into a host cell. Any suitable method may be used to introduce a nucleic acid into a cell. Suitable methods include, for example, viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. Further methods are described throughout.

Introducing one or more nucleic acids into a host cell may occur in any culture media and under any culture conditions that promote the survival of the cells. Introducing one or more nucleic acids into a host cell may be carried out in vivo or ex vivo. Introducing one or more nucleic acids into a host cell may be carried out in vitro.

In some embodiments, an effector protein may be provided as RNA. The RNA may be provided by direct chemical synthesis or may be transcribed in vitro from a DNA (e.g., encoding the effector protein). Once synthesized, the RNA may be introduced into a cell by way of any suitable technique for introducing nucleic acids into cells (e.g., microinjection, electroporation, transfection, etc.). In some embodiments, introduction of one or more nucleic acid may be through the use of a vector and/or a vector system, accordingly, in some embodiments, compositions, devices, kits, methods and systems described herein comprise a vector and/or a vector system.

Vectors may be introduced directly to a host. In some embodiments, host cells may be contacted with one or more vectors as described herein, and in some embodiments, said vectors are taken up by the cells. Methods for contacting cells with vectors include but are not limited to electroporation, calcium chloride transfection, microinjection, lipofection, micro-injection, contact with the cell or particle that comprises a molecule of interest, or a package of cells or particles that comprise molecules of interest.

Components described herein may also be introduced directly to a host. For example, an engineered guide nucleic acid may be introduced to a host, specifically introduced into a host cell. Methods of introducing nucleic acids, such as RNA into cells include, but are not limited to direct injection, transfection, or any other method used for the introduction of nucleic acids.

Polypeptides (e.g., effector proteins) described herein may also be introduced directly to a host. In some embodiments, polypeptides described herein may be modified to promote introduction to a host. For example, polypeptides described herein may be modified to increase the solubility of the polypeptide. Such a polypeptide may optionally be fused to a polypeptide domain that increases solubility. The domain may be linked to the polypeptide through a defined protease cleavage site, such as TEV sequence which is cleaved by TEV protease. The linker may also include one or more flexible sequences, e.g., from 1 to 10 glycine residues. In some embodiments, the cleavage of the polypeptide is performed in a buffer that maintains solubility of the product, e.g., in the presence of from 0.5 to 2 M urea, in the presence of polypeptides and/or polynucleotides that increase solubility, and the like. Domains of interest include endosomolytic domains, e.g., influenza HA domain; and other polypeptides that aid in production, e.g., IF2 domain, GST domain, GRPE domain, and the like. In another example, the polypeptide may be modified to improve stability. For example, the polypeptides may be PEGylated, where the polyethyleneoxy group provides for enhanced lifetime in the blood stream. Polypeptides may also be modified to promote uptake by a host, such as a host cell. For example, a polypeptide described herein may be fused to a polypeptide permeant domain to promote uptake by a host cell. Any suitable permeant domains may be used in the non-integrating polypeptides of the present disclosure, including peptides, peptidomimetics, and non-peptide carriers. Examples include penetratin, a permeant peptide may be derived from the third alpha helix of Drosophila melanogaster transcription factor Antennapaedia; the HIV-1 tat basic region amino acid sequence, e.g., amino acids 49-57 of a naturally-occurring tat protein; and poly-arginine motifs, for example, the region of amino acids 34-56 of HIV-1 rev protein, nonaarginine, octa-arginine, and the like. The site at which the fusion is made may be selected in order to optimize the biological activity, secretion or binding characteristics of the polypeptide. The optimal site may be determined by suitable methods.

### Formulations for Introducing System Components and Compositions to a Host

Described herein are formulations of introducing compositions or components of a system described herein to a host. In some embodiments, such formulations, systems and compositions described herein comprise an effector protein and a carrier (e.g., excipient, diluent, vehicle, or filling agent). In some aspects of the present disclosure, the effector protein is provided in a pharmaceutical composition comprising the effector protein and any pharmaceutically acceptable excipient, carrier, or diluent.

### VIII. Modification of Target Nucleic Acids

Provided herein are methods of modifying (e.g., editing) target nucleic acids or the expression thereof. Also disclosed herein are compositions, systems, devices, and kits for modifying a target nucleic acid. In general, modifying refers to changing the physical composition of a target nucleic acid. However, compositions, methods, and systems disclosed herein may also be capable of modifying target nucleic acids, such as making epigenetic modifications of target nucleic acids, which does not change the nucleotide sequence of the target nucleic acids per se. Effector proteins, compositions and systems described herein may be used for modifying a target nucleic acid, which includes editing a target nucleic acid sequence. In some embodiments, methods comprise editing a target nucleic acid. In general, editing refers to modifying the nucleobase sequence of a target nucleic acid. Modifying a target nucleic acid may comprise one or more of: cleaving the target nucleic acid, deleting one or more nucleotides of the target nucleic acid, inserting one or more nucleotides into the target nucleic acid, mutating one or more nucleotides of the target nucleic acid, or otherwise changing one or more nucleotides of the target nucleic acid. Modifying a target nucleic acid may comprise one or more of: methylating, demethylating, deaminating, or oxidizing one or more nucleotides of the target nucleic acid.

Also provided herein are methods, compositions, systems, kits and devices of modulating the expression of a target nucleic acid. Fusion effector proteins and systems described herein may be used for such methods. Methods of editing a target nucleic acid may comprise one or more of cleaving the target nucleic acid, deleting one or more nucleotides of the target nucleic acid, inserting one or more nucleotides into the target nucleic acid, modifying one or more nucleotides of the target nucleic acid. Methods of modulating expression of target nucleic acids may comprise modifying the target nucleic acid or a protein associated with the target nucleic acid, e.g., a histone. Methods of modulating expression of target nucleic acids may comprise insertion of one or more nucleotides into the target nucleic acid, wherein the one or more nucleotide can be referred to as a donor nucleotide. Therefore, the term “donor nucleotide” as used herein can refer to a single nucleotide that is incorporated into a target nucleic acid. A nucleotide is typically inserted at a site of cleavage by an effector protein.

The target nucleic acid may be a gene or a portion thereof. Methods, devices, kits, systems and compositions may modify a coding portion of a gene, a non-coding portion of a gene, or a combination thereof. Modifying at least one gene using the compositions, devices, kits, systems, and methods described herein may reduce or increase expression of one or more genes. In some embodiments, compositions, devices, kits, systems and methods reduce expression of one or more genes by at least 10%, at least 20%, at least 30%, at least 40%, at least 50%, at least 60%, at least 70%, at least 80%, at least 90%, or at least 95%. In some embodiments, compositions, systems, devices, kits and methods remove all expression of a gene, also referred to as genetic knock out. In some embodiments, compositions, devices, kits, systems, and methods increase expression of one or more genes by at least 10%, at least 20%, at least 30%, at least 40%, at least 50%, at least 60%, at least 70%, at least 80%, at least 90%, or at least 100%.

In some embodiments, compositions, devices, kits, systems and methods use effector proteins that are fused to a heterologous protein. Heterologous proteins include, but are not limited to, transcriptional activators, transcriptional repressors, deaminases, methyltransferases, acetyltransferases, and other nucleic acid modifying proteins. In some cases, effector proteins need not be fused to a partner protein to accomplish the required protein (expression) modification.

In some embodiments, compositions, devices, kits, systems and methods comprise a nucleic acid expression vector, or use thereof, to introduce an effector protein, guide nucleic acid, donor template or any combination thereof to a cell. In some embodiments, the nucleic acid expression vector is a viral vector. Viral vectors include, but are not limited to, retroviruses, adenoviruses, adeno-associated viruses, and herpes simplex viruses. In some embodiments, the viral vector is a replication-defective viral vector, comprising an insertion of a therapeutic gene inserted in genes essential to the lytic cycle, preventing the virus from replicating and exerting cytotoxic effects. In some embodiments, the viral vector is an adeno associated viral (AAV) vector. In some embodiments, the nucleic acid expression vector is a non-viral vector. In some embodiments, compositions, devices, kits, systems and methods comprise a lipid, polymer, nanoparticle, or a combination thereof, or use thereof, to introduce an effector protein, guide nucleic acid, donor template or any combination thereof to a cell. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

In some embodiments, methods comprise contacting a target nucleic acid with a composition, device, kit, or system described herein. In some embodiments, methods comprise contacting a target nucleic acid with an effector protein described herein. In some embodiments, methods comprise contacting a target nucleic acid with a fusion effector protein described herein. The effector protein may be an effector protein provided in TABLE 1 or a catalytically inactive variant thereof. The effector protein may comprise an amino acid sequence that is at least 70%, at least 75%, at least 80%, at least 85%, at least 90%, at least 95%, at least 97%, at least 98%, at least 99% or 100% identical to a sequence described in TABLE 1. In some embodiments, the amino acid sequence of the effector protein is at least 70%, at least 75%, at least 80%, at least 85%, at least 90%, at least 95%, at least 97%, at least 98%, at least 99% or 100% identical to a sequence described in TABLE 1.

Methods of modifying may comprise contacting a target nucleic acid with one or more components, compositions or systems described herein. In some embodiments, a method of modifying comprises contacting a target nucleic acid with at least one of: a) one or more effector proteins, or one or more nucleic acids encoding one or more effector proteins; or b) one or more guide nucleic acids, or one or more nucleic acids encoding one or more guide nucleic acids. In some embodiments, a method of modifying comprises contacting a target nucleic acid with a system described herein wherein the system comprises components comprising at least one of: a) one or more effector proteins, or one or more nucleic acids encoding one or more effector proteins; or b) one or more guide nucleic acids, or one or more nucleic acids encoding one or more guide nucleic acids. In some embodiments, a method of modifying comprises contacting a target nucleic acid with a composition described herein comprising at least one of: a) one or more effector proteins, or one or more nucleic acids encoding one or more effector proteins; or b) one or more guide nucleic acids, or one or more nucleic acids encoding one or more guide nucleic acids; in a composition.

In some embodiments, methods comprise base editing. In some embodiments, base editing comprises contacting a target nucleic acid with a fusion effector protein comprising an effector protein fused to a base editing enzyme, such as a deaminase, thereby changing a nucleobase of the target nucleic acid to an alternative nucleobase. In some embodiments, the nucleobase of the target nucleic acid is adenine (A), and the method comprises changing A to guanine (G). In some embodiments, the nucleobase of the target nucleic acid is cytosine (C), and the method comprises changing C to thymine (T). In some embodiments, the nucleobase of the target nucleic acid is C, and the method comprises changing C to G. In some embodiments, the nucleobase of the target nucleic acid is A, and the method comprises changing A to G.

In some embodiments, methods introduce a nucleobase change in a target nucleic acid relative to a corresponding wildtype or mutant nucleobase sequence. In some embodiments, methods remove or correct a disease-causing mutation in a nucleic acid sequence, e.g., to produce a corresponding wildtype nucleobase sequence. In some embodiments, methods remove/correct point mutations, deletions, null mutations, or tissue-specific mutations in a target nucleic acid. In some embodiments, methods generate gene knock-out, gene knock-in, gene editing, gene tagging, or a combination thereof. Methods of the disclosure may be targeted to a locus in a genome of a cell.

Editing a target nucleic acid sequence may introduce a mutation (e.g., point mutations, deletions) in a target nucleic acid relative to a corresponding wildtype nucleotide sequence. Editing may remove or correct a disease-causing mutation in a nucleic acid sequence to produce a corresponding wildtype nucleotide sequence. Editing a target nucleic acid sequence may remove/correct point mutations, deletions, null mutations, or tissue-specific mutations in a target nucleic acid. Editing a target nucleic acid sequence may be used to generate gene knock-out, gene knock-in, gene editing, gene tagging, or a combination thereof. Methods of the disclosure may be targeted to any locus in a genome of a cell.

Modifying may comprise single stranded cleavage, double stranded cleavage, donor nucleic acid insertion, epigenetic modification (e.g., methylation, demethylation, acetylation, or deacetylation), or a combination thereof. In some embodiments, cleavage (single-stranded or double-stranded) is site-specific, meaning cleavage occurs at a specific site in the target nucleic acid, often within the region of the target nucleic acid that hybridizes with the guide nucleic acid spacer sequence. In some embodiments, the effector proteins introduce a single-stranded break in a target nucleic acid to produce a cleaved nucleic acid. In some embodiments, the effector protein is capable of introducing a break in a single stranded RNA (ssRNA). The effector protein may be coupled to a guide nucleic acid that targets a particular region of interest in the ssRNA. In some embodiments, the target nucleic acid, and the resulting cleaved nucleic acid is contacted with a nucleic acid for homologous recombination (e.g., homology directed repair (HDR)) or non-homologous end joining (NHEJ). In some embodiments, a double-stranded break in the target nucleic acid may be repaired (e.g., by NHEJ or HDR) without insertion of a donor template, such that the repair results in an indel in the target nucleic acid at or near the site of the double-stranded break. In some embodiments, an indel, sometimes referred to as an insertion-deletion or indel mutation, is a type of genetic mutation that results from the insertion and/or deletion of one or more nucleotide in a target nucleic acid. An indel may vary in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation. Indel percentage is the percentage of sequencing reads that show at least one nucleotide has been mutation that results from the insertion and/or deletion of nucleotides regardless of the size of insertion or deletion, or number of nucleotides mutated. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are edited by a given effector protein.

In some embodiments, methods of modifying described herein cleave a target nucleic acid at one or more locations to generate a cleaved target nucleic acid. In some embodiments, the cleaved target nucleic acid undergoes recombination (e.g., NHEJ or HDR). In some embodiments, cleavage in the target nucleic acid may be repaired (e.g., by NHEJ or HDR) without insertion of a donor nucleic acid, such that the repair results in an indel in the target nucleic acid at or near the site of the cleavage site. In some embodiments, cleavage in the target nucleic acid may be repaired (e.g., by NHEJ or HDR) with insertion of a donor nucleic acid, such that the repair results in an indel in the target nucleic acid at or near the site of the cleavage site.

In some embodiments, wherein the compositions, systems, and methods of the present disclosure comprise an additional guide nucleic acid or a use thereof, and such dual-guided compositions, systems, and methods described herein may modify the target nucleic acid in two locations. In some embodiments, dual-guided modifying may comprise cleavage of the target nucleic acid in the two locations targeted by the guide nucleic acids. In some embodiments, upon removal of the sequence between the guide nucleic acids, the wild-type reading frame is restored. A wild-type reading frame may be a reading frame that produces at least a partially, or fully, functional protein. A non-wild-type reading frame may be a reading frame that produces a non-functional or partially non-functional protein.

Accordingly, in some embodiments, compositions, systems, and methods described herein may edit 1 to 1,000 nucleotides or any integer in between, in a target nucleic acid. In some embodiments, 1 to 1,000, 2 to 900, 3 to 800, 4 to 700, 5 to 600, 6 to 500, 7 to 400, 8 to 300, 9 to 200, or 10 to 100 nucleotides, or any integer in between, may be edited by the compositions, systems, and methods described herein. In some embodiments, 1, 2, 3, 4, 5, 6, 7, 8, 9, 10, or more nucleotides may be edited by the compositions, systems, and methods described herein. In some embodiments, 10, 20, 30, 40, 50, 60, 70, 80 90, 100 or more nucleotides, or any integer in between, may be edited by the compositions, systems, and methods described herein. In some embodiments, 100, 200, 300, 400, 500, 600, 700, 800, 900 or more nucleotides, or any integer in between, may be edited by the compositions, systems, and methods described herein.

Methods may comprise use of two or more effector proteins. An illustrative method for introducing a break in a target nucleic acid comprises contacting the target nucleic acid with: (a) a first engineered guide nucleic acid comprising a region that binds to a first effector protein described herein; and (b) a second engineered guide nucleic acid comprising a region that binds to a second effector protein described herein, wherein the first engineered guide nucleic acid comprises an additional region that hybridizes to the target nucleic acid and wherein the second engineered guide nucleic acid comprises an additional region that hybridizes to the target nucleic acid. In some embodiments, the first and second effector protein are identical. In some embodiments, the first and second effector protein are not identical.

In some embodiments, editing a target nucleic acid comprises genome editing. Genome editing may comprise editing a genome, chromosome, plasmid, or other genetic material of a cell or organism. In some embodiments, the genome, chromosome, plasmid, or other genetic material of the cell or organism is modified in vivo. In some embodiments, the genome, chromosome, plasmid, or other genetic material of the cell or organism is modified in a cell. In some embodiments, the genome, chromosome, plasmid, or other genetic material of the cell or organism is modified in vitro. For example, a plasmid may be edited in vitro using a composition described herein and introduced into a cell or organism.

In some embodiments, editing a target nucleic acid may comprise deleting a sequence from a target nucleic acid. For example, a mutated sequence or a sequence associated with a disease may be removed from a target nucleic acid. In some embodiments, editing a target nucleic acid may comprise replacing a sequence in a target nucleic acid with a second sequence. For example, a mutated sequence or a sequence associated with a disease may be replaced with a second sequence lacking the mutation or that is not associated with the disease. In some embodiments, editing a target nucleic acid may comprise introducing a sequence into a target nucleic acid. For example, a beneficial sequence or a sequence that may reduce or eliminate a disease may be inserted into the target nucleic acid.

In some embodiments, methods comprise inserting a donor nucleic acid into a cleaved target nucleic acid. The donor nucleic acid may be inserted at a specified (e.g., effector protein targeted) point within the target nucleic acid. In some embodiments, the cleaved target nucleic acid is cleaved at a single location. In such embodiments, the methods comprise contacting a target nucleic acid with an effector protein described herein, thereby introducing a single-stranded break in the target nucleic acid; and contacting the target nucleic acid with a donor nucleic acid for homologous recombination, optionally by HDR or NHEJ, thereby introducing a new sequence into the target nucleic acid (e.g., at a cleavage site). In some embodiments, the cleaved target nucleic acid is cleaved at two locations. In such embodiments, the methods comprise contacting a target nucleic acid with an effector protein described herein, thereby introducing a single-stranded break in the target nucleic acid; contacting the target nucleic acid with a second effector protein described herein, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally by HDR or NHEJ, thereby introducing a new sequence into the target nucleic acid (e.g., in between two cleavage sites).

In some embodiments, methods comprise editing a target nucleic acid with two or more effector proteins. Editing a target nucleic acid may comprise introducing a two or more single-stranded breaks in a target nucleic acid. In some embodiments, a break may be introduced by contacting a target nucleic acid with an effector protein and a guide nucleic acid. The guide nucleic acid may bind to the effector protein and hybridize to a region of the target nucleic acid, thereby recruiting the effector protein to the region of the target nucleic acid. Binding of the effector protein to the guide nucleic acid and the region of the target nucleic acid may activate the effector protein, and the effector protein may introduce a break (e.g., a single stranded break) in the region of the target nucleic acid. In some embodiments, editing a target nucleic acid may comprise introducing a first break in a first region of the target nucleic acid and a second break in a second region of the target nucleic acid. For example, editing a target nucleic acid may comprise contacting a target nucleic acid with a first guide nucleic acid that binds to a first effector protein and hybridizes to a first region of the target nucleic acid and a second guide nucleic acid that binds to a second programmable nickase and hybridizes to a second region of the target nucleic acid. The first effector protein may introduce a first break in a first strand at the first region of the target nucleic acid, and the second effector protein may introduce a second break in a second strand at the second region of the target nucleic acid. In some embodiments, a segment of the target nucleic acid between the first break and the second break may be removed, thereby editing the target nucleic acid. In some embodiments, a segment of the target nucleic acid between the first break and the second break may be replaced (e.g., with donor nucleic acid), thereby editing the target nucleic acid.

Methods, systems and compositions described herein may edit a target nucleic acid wherein such editing may effect one or more indels. In some embodiments, where compositions, systems, and/or methods described herein effect one or more indels, the impact on the transcription and/or translation of the target nucleic acid may be predicted depending on: 1) the amount of indels generated; and 2) the location of the indel on the target nucleic acid. For example, as described herein, in some embodiments, if the amount of indels is not divisible by three, and the indels occur within or along a protein coding region, then the edit or mutation may be a frameshift mutation. In some embodiments, if the amount of indels is divisible by three, then a frameshift mutation may not be effected, but a splicing disruption mutation and/or sequence skip mutation may be effected, such as an exon skip mutation. In some embodiments, if the amount of indels is not evenly divisible by three, then a frameshift mutation may be effected.

Methods, systems and compositions described herein may edit a target nucleic acid wherein such editing may be measured by indel activity. Indel activity measures the amount of change in a target nucleic acid (e.g., nucleotide deletion(s) and/or insertion(s)) compared to a target nucleic acid that has not been contacted by a polypeptide described in compositions, systems, and methods described herein. For example, indel activity may be detected by next generation sequencing of one or more target loci of a target nucleic acid where indel percentage is calculated as the fraction of sequencing reads containing insertions or deletions relative to an unedited reference sequence. In some embodiments, methods, systems, and compositions comprising an effector protein and guide nucleic acid described herein may exhibit about 0.0001% to about 65% or more indel activity upon contact to a target nucleic acid compared to a target nucleic acid non-contacted with compositions, systems, or by methods described herein. For example, methods, systems, and compositions comprising an effector protein and guide nucleic acid described herein may exhibit about 0.0001%, about 0.001%, about 0.01%, about 0.1%, about 1%, about 5%, about 10%, about 15%, about 20%, about 25%, about 30%, about 35%, about 40%, about 45%, about 50%, about 55%, about 60%, about 65% or more indel activity.

In some embodiments, editing of a target nucleic acid as described herein effects one or more mutations comprising splicing disruption mutations, frameshift mutations (e.g., 1+ or 2+ frameshift mutation), sequence deletion, sequence skipping, sequence reframing, sequence knock-in, or any combination thereof. In some embodiments, the splicing disruption can be an editing that disrupts a splicing of a target nucleic acid or a splicing of a sequence that is transcribed from a target nucleic acid relative to a target nucleic acid without the splicing disruption. In some embodiments, the frameshift mutation can be an editing that alters the reading frame of a target nucleic acid relative to a target nucleic acid without the frameshift mutation. In some embodiments, the frameshift mutation can be a +2 frameshift mutation, wherein a reading frame is edited by 2 bases. In some embodiments, the frameshift mutation can be a +1 frameshift mutation, wherein a reading frame is edited by 1 base. In some embodiments, the frameshift mutation is an editing that alters the number of bases in a target nucleic acid so that it is not divisible by three. In some embodiments, the frameshift mutation can be an editing that is not a splicing disruption. In some embodiments a sequence as described in reference to the sequence deletion, sequence skipping, sequence reframing, and sequence knock-in can be a DNA sequence, a RNA sequence, an edited DNA or RNA sequence, a mutated sequence, a wild-type sequence, a coding sequence, a non-coding sequence, an exonic sequence (exon), an intronic sequence (intron), or any combination thereof. In some embodiments, the sequence deletion is an editing where one or more sequences in a target nucleic acid are deleted relative to a target nucleic acid without the sequence deletion. In some embodiments, the sequence deletion can result in or effect a splicing disruption or a frameshift mutation. In some embodiments, the sequence deletion result in or effect a splicing disruption. In some embodiments, the sequence skipping is an editing where one or more sequences in a target nucleic acid are skipped upon transcription or translation of the target nucleic acid relative to a target nucleic acid without the sequence skipping. In some embodiments, the sequence skipping can result in or effect a splicing disruption or a frameshift mutation. In some embodiments, the sequence skipping can result in or effect a splicing disruption. In some embodiments, the sequence reframing is an editing where one or more bases in a target are edited so that the reading frame of the sequence is reframed relative to a target nucleic acid without the sequence reframing. In some embodiments, the sequence reframing can result in or effect a splicing disruption or a frameshift mutation. In some embodiments, the sequence reframing can result in or effect a frameshift mutation. In some embodiments, the sequence knock-in is an editing where one or more sequences is inserted into a target nucleic acid relative to a target nucleic acid without the sequence knock-in. In some embodiments, the sequence knock-in can result in or effect a splicing disruption or a frameshift mutation. In some embodiments, the sequence knock-in can result in or effect a splicing disruption.

In some embodiments, editing of a target nucleic acid can be locus specific, wherein compositions, systems, and methods described herein can edit a target nucleic acid at one or more specific loci to effect one or more specific mutations comprising splicing disruption mutations, frameshift mutations, sequence deletion, sequence skipping, sequence reframing, sequence knock-in, or any combination thereof. For example, editing of a specific locus can affect any one of a splicing disruption, frameshift (e.g., 1+ or 2+ frameshift), sequence deletion, sequence skipping, sequence reframing, sequence knock-in, or any combination thereof. In some embodiments, editing of a target nucleic acid can be locus specific, modification specific, or both. In some embodiments, editing of a target nucleic acid can be locus specific, modification specific, or both, wherein compositions, systems, and methods described herein comprise an effector protein described herein and a guide nucleic acid described herein.

In some embodiments, methods of editing a target nucleic acid or modulating the expression of a target nucleic acid are performed in vivo. In some embodiments, methods of editing a target nucleic acid or modulating the expression of a target nucleic acid are performed in vitro. For example, a plasmid may be modified in vitro using a composition, devices, kits, or system, or method described herein and introduced into a cell or organism. In some embodiments, methods of editing a target nucleic acid or modulating the expression of a target nucleic acid are performed ex vivo. For example, methods may comprise obtaining a cell from a subject, modifying a target nucleic acid in the cell with methods, devices, kits, systems and compositions described herein, and returning the cell to the subject. Methods of editing performed ex vivo may be particularly advantageous to produce CAR T-cells. In some embodiments, methods comprise editing a target nucleic acid or modulating the expression of the target nucleic acid in a cell or a subject. The cell may be a dividing cell. The cell may be a terminally differentiated cell. In some embodiments, the target nucleic acid is a gene.

In some embodiments, methods of modifying described herein comprise contacting a target nucleic acid with one or more components, compositions or systems described herein. In some embodiments, the one or more components, compositions or systems described herein comprise at least one of: a) one or more effector proteins, or one or more nucleic acids encoding one or more effector proteins; and b) one or more guide nucleic acids, or one or more nucleic acids encoding one or more guide nucleic acids. In some embodiments, the one or more effector proteins introduce a single-stranded break or a double-stranded break in the target nucleic acid.

In some embodiments, methods of modifying described herein comprise using one or more guide nucleic acids or uses thereof, wherein the methods modify a target nucleic acid at a single location. In some embodiments, the methods comprise contacting an RNP comprising an effector protein and a guide nucleic acid to the target nucleic acid. In some embodiments, the methods introduce a mutation (e.g., point mutations, deletions) in the target nucleic acid relative to a corresponding wildtype nucleotide sequence. In some embodiments, the methods remove or correct a disease-causing mutation in a nucleic acid sequence to produce a corresponding wildtype nucleotide sequence. In some embodiments, the methods remove/correct point mutations, deletions, null mutations, or tissue-specific mutations in a target nucleic acid. In some embodiments, the methods introduce a single stranded cleavage, a nick, a deletion of one or two nucleotides, an insertion of one or two nucleotides, a substitution of one or two nucleotides, an epigenetic modification (e.g., methylation, demethylation, acetylation, or deacetylation), or a combination thereof to the target nucleic acid. In some embodiments, the methods comprise using an effector protein and two guide nucleic acids, wherein two RNPs cleave the target nucleic acid at the same location, wherein a first RNP comprises the effector protein and a first guide nucleic acid, and wherein a second RNP comprises the effector protein and a second guide nucleic acid. In some embodiments, methods comprising using two effector protein and two guide nucleic acids, wherein both RNPs cleave the target nucleic acid at the same location, wherein a first RNP comprises a first effector protein and a first target nucleic acid, and wherein a second RNP comprises a second effector protein and a second target nucleic acid.

In some embodiments, methods of modifying described herein comprise using one or more guide nucleic acids or uses thereof, wherein the methods modify a target nucleic acid at two different locations. In some embodiments, the methods introduce two cleavage sites in the target nucleic acid, wherein a first cleavage site and a second cleavage site comprise one or more nucleotides therebetween. In some embodiments, the methods cause deletion of the one or more nucleotides. In some embodiments, the deletion restores a wild-type reading frame. In some embodiments, the wild-type reading frame produces at least a partially functional protein. In some embodiments, the deletion causes a non-wild-type reading frame. In some embodiments, a non-wild-type reading frame produces a partially functional protein or non-functional protein. In some embodiments, the at least partially functional protein has at least 50%, at least 60%, at least 70%, at least 80%, at least 90%, at least 100%, at least 110%, at least 120%, at least 130%, at least 140%, at least 150%, at least 180%, at least 200%, at least 300%, at least 400% activity compared to a corresponding wildtype protein. In some embodiments, the methods comprise using an effector protein and two guide nucleic acids, wherein two RNPs cleave the target nucleic acid at different locations, wherein a first RNP comprises the effector protein and a first guide nucleic acid, and wherein a second RNP comprises the effector protein and a second guide nucleic acid. In some embodiments, methods comprising using two effector protein and two guide nucleic acids, wherein both RNPs cleave the target nucleic acid at the same location, wherein a first RNP comprises a first effector protein and a first target nucleic acid, and wherein a second RNP comprises a second effector protein and a second target nucleic acid.

In some embodiments, methods of editing described herein comprise inserting a donor nucleic acid into a cleaved target nucleic acid. In some embodiments, the cleaved target nucleic acid formed by introducing a single-stranded break into a target nucleic acid. The donor nucleic acid may be inserted at a specified (e.g., effector protein targeted) point within the target nucleic acid. In some embodiments, the cleaved target nucleic acid is cleaved at a single location. In such embodiments, the methods comprise contacting a target nucleic acid with an effector protein described herein, thereby introducing a single-stranded break in the target nucleic acid; and contacting the target nucleic acid with a donor nucleic acid for homologous recombination, optionally by HDR or NHEJ, thereby introducing a new sequence into the target nucleic acid (e.g., at a cleavage site). In some embodiments, the cleaved target nucleic acid is cleaved at two locations. In such embodiments, the methods comprise contacting a target nucleic acid with an effector protein described herein, thereby introducing a single-stranded break in the target nucleic acid; contacting the target nucleic acid with a second effector protein described herein, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally by HDR or NHEJ, thereby introducing a new sequence into the target nucleic acid (e.g., in between two cleavage sites).

### Donor Nucleic Acids

In some embodiments, a donor nucleic acid comprises a nucleic acid that is incorporated into a target nucleic acid or genome. In some embodiments, a donor nucleic acid comprises a sequence that is derived from a plant, bacteria, fungi, virus, or an animal. In some embodiments, the animal is a non-human animal, such as, by way of non-limiting example, a mouse, rat, hamster, rabbit, pig, bovine, deer, sheep, goat, chicken, cat, dog, ferret, a bird, non-human primate (e.g., marmoset, rhesus monkey). In some embodiments, the non-human animal is a domesticated mammal or an agricultural mammal. In some embodiments, the animal is a human. In some embodiments, the sequence comprises a human wild-type (WT) gene or a portion thereof. In some embodiments, the human WT gene or the portion thereof comprises a nucleotide sequence that is at least 70%, at least 80%, at least 90%, at least 92%, at least 95%, at least 97%, at least 99%, or 100% identical to an equal length portion of the WT sequence of any one of the sequences recited in TABLE 7. In some embodiments, the donor nucleic acid is incorporated into an insertion site of a target nucleic acid.

In some embodiments, the donor nucleic acid comprises single-stranded DNA or linear double-stranded DNA. In some embodiments, the donor nucleic acid comprises a nucleotide sequence encoding a functional polypeptide and/or wherein the donor nucleic acid comprises a wildtype sequence. In some embodiments, the donor nucleic acid comprises a protein coding sequence, a gene, a gene fragment, an exon, an intron, an exon fragment, an intron fragment, a gene regulatory fragment, a gene regulatory region fragment, coding sequences thereof, or combinations thereof. In some embodiments, the donor nucleic acid comprises a naturally occurring sequence. In some embodiments, the naturally occurring sequence does not contain a mutation.

In some embodiments, the donor nucleic acid comprises a gene fragment, an exon fragment, an intron fragment, a gene regulatory region fragment, or combinations thereof. In some embodiments, the fragment is at least 10, at least 20, at least 30, at least 40, at least 50, at least 60, at least 70, or at least 80 contiguous nucleotides.

In some embodiments, a donor nucleic acid of any suitable size is integrated into a target nucleic acid or a genome. In some embodiments, the donor nucleic acid integrated into the target nucleic acid or the genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 kilobases in length. In some embodiments, the donor nucleic acid is more than 500 kilobases (kb) in length.

In some embodiments, a viral vector comprising a donor nucleic acid introduces the donor nucleic acid into a cell following transfection. In some embodiments, the donor nucleic acid is introduced into the cell by any mechanism of the transfecting viral vector, including, but not limited to, integration into the genome of the cell or introduction of an episomal plasmid or viral genome.

In some embodiments, an effector protein as described herein facilitates insertion of a donor nucleic acid at a site of cleavage or between two cleavage sites by cleaving (hydrolysis of a phosphodiester bond) of a nucleic acid resulting in a nick or double strand break-nuclease activity.

In some embodiments, a donor nucleic acid serves as a template in the process of homologous recombination, which may carry an alteration that is to be or has been introduced into a target nucleic acid. By using the donor nucleic acid as a template, the genetic information, including the alteration, is copied into the target nucleic acid by way of homologous recombination.

### Genetically Modified Cells and Organisms

Methods of editing a target nucleic acid or modulating the expression of a target nucleic acid described herein may be employed to generate a genetically modified cell. The cell may be a prokaryotic cell. The cell may be an archaeal cell. The cell may be a eukaryotic cell. The cell may be a mammalian cell. The cell may be a human cell. The cell may be a T cell. The cell may be a hematopoietic stem cell. The cell may be a bone marrow derived cell, a white blood cell, a blood cell progenitor, or a combination thereof. In some embodiments, the cell is derived from a multicellular organism and cultured as a unicellular entity. In some embodiments, the cell comprises a heritable genetic modification, such that progeny cells derived therefrom comprise the heritable genetic mutation. In some embodiments, the cell is progeny of a genetically modified cell comprising a genetic modification of the genetically modified parent cell. In some embodiments, the genetically modified cell comprises a deletion, insertion, mutation, or non-native sequence relative to a wild-type version of the cell or the organism from which the cell was derived.

Methods of editing described herein may be performed in a cell. In some embodiments, the cell is in vitro. In some embodiments, the cell is in vivo. In some embodiments, the cell is ex vivo. In some embodiments, the cell is an isolated cell. In some embodiments, the cell is inside of an organism. In some embodiments, the cell is an organism. In some embodiments, the cell is in a cell culture. In some embodiments, the cell is one of a collection of cells. In some embodiments, the cell is a mammalian cell or derived there from. In some embodiments, the cell is a rodent cell or derived there from. In some embodiments, the cell is a human cell or derived there from. In some embodiments, the cell is a eukaryotic cell or derived there from. In some embodiments, the cell is a progenitor cell or derived there from. In some embodiments, the cell is a pluripotent stem cell or derived there from. In some embodiments, the cell is an animal cell or derived there from. In some embodiments, the cell is an invertebrate cell or derived there from. In some embodiments, the cell is a vertebrate cell or derived there from. In some embodiments, the cell is from a specific organ or tissue. In some embodiments, the cell is a hepatocyte. In some embodiments, the tissue is a subject's blood, bone marrow, or cord blood. In some embodiments, the tissue is a heterologous donor blood, cord blood, or bone marrow. In some embodiments, the tissue is an allogenic blood, cord blood, or bone marrow. In some embodiments, the tissue may be muscle. In some embodiments, the muscle may be a skeletal muscle. In some embodiments, skeletal muscles include the following: abductor digiti minimi (foot), abductor digiti minimi (hand), abductor hallucis, abductor pollicis brevis, abductor pollicis longus, adductor brevis, adductor hallucis, adductor longus, adductor magnus, adductor pollicis, anconeus, articularis cubiti, articularis genu, aryepiglotticus, auricularis, biceps brachii, biceps femoris, brachialis, brachioradialis, buccinator, bulbospongiosus, constrictor of pharynx-inferior, constrictor of pharynx-middle, constrictor of pharynx-superior, coracobrachialis, corrugator supercilii, cremaster, cricothyroid, dartos, deep transverse perinei, deltoid, depressor anguli oris, depressor labii inferioris, diaphragm, digastric, digastric (anterior view), erector spinae-spinalis, erector spinae-iliocostalis, erector spinae-longissimus, extensor carpi radialis brevis, extensor carpi radialis longus, extensor carpi ulnaris, extensor digiti minimi (hand), extensor digitorum (hand), extensor digitorum brevis (foot), extensor digitorum longus (foot), extensor hallucis brevis, extensor hallucis longus, extensor indicis, extensor pollicis brevis, extensor pollicis longus, external oblique abdominis, flexor carpi radialis, flexor carpi ulnaris, flexor digiti minimi brevis (foot), flexor digiti minimi brevis (hand), flexor digitorum brevis, flexor digitorum longus (foot), flexor digitorum profundus, flexor digitorum superficialis, flexor hallucis brevis, flexor hallucis longus, flexor pollicis brevis, flexor pollicis longus, frontalis, gastrocnemius, gemellus inferior, gemellus superior, genioglossus, geniohyoid, gluteus maximus, gluteus medius, gluteus minimus, gracilis, hyoglossus, iliacus, inferior oblique, inferior rectus, infraspinatus, intercostals external, intercostals innermost, intercostals internal, internal oblique abdominis, interossei-dorsal of hand, interossei-dorsal of foot, interossei-palmar of hand, interossei-plantar of foot, interspinales, intertransversarii, intrinsic muscles of tongue, ishiocavernosus, lateral cricoarytenoid, lateral pterygoid, lateral rectus, latissimus dorsi, levator anguli oris, levator ani-coccygeus, levator ani-iliococcygeus, levator ani-pubococcygeus, levator ani-puborectalis, levator ani-pubovaginalis, levator labii superioris, levator labii superioris, alaeque nasi, levator palpebrae superioris, levator scapulae, levator veli palatini, levatores costarum, longus capitis, longus colli, lumbricals of foot, lumbricals of hand, masseter, medial pterygoid, medial rectus, mentalis, m. uvulae, mylohyoid, nasalis, oblique arytenoid, obliquus capitis inferior, obliquus capitis superior, obturator externus, obturator internus (A), obturator internus (B), omohyoid, opponens digiti minimi (hand), opponens pollicis, orbicularis oculi, orbicularis oris, palatoglossus, palatopharyngeus, palmaris brevis, palmaris longus, pectineus, pectoralis major, pectoralis minor, peroneus brevis, peroneus longus, peroneus tertius, piriformis (A), piriformis (B), plantaris, platysma, popliteus, posterior cricoarytenoid, procerus, pronator quadratus, pronator teres, psoas major, psoas minor, pyramidalis, quadratus femoris, quadratus lumborum, quadratus plantae, rectus abdominis, rectus capitus anterior, rectus capitus lateralis, rectus capitus posterior major, rectus capitus posterior minor, rectus femoris, rhomboid major, rhomboid minor, risorius, salpingopharyngeus, sartorius, scalenus anterior, scalenus medius, scalenus minimus, scalenus posterior, semimembranosus, semitendinosus, serratus anterior, serratus posterior inferior, serratus posterior superior, soleus, sphincter ani, sphincter urethrae, splenius capitis, splenius cervicis, stapedius, sternocleidomastoideohyoid, sternothyroid, styloglossus, stylohyoid, stylohyoid (anterior view), stylopharyngeus, subclavius, subcostalis, subscapularis, superficial transverse perinei, superior oblique, superior rectus, supinator, supraspinatus, temporalis, temporoparietalis, tensor fasciae lata, tensor tympani, tensor veli palatini, teres major, teres minor, thyro-arytenoid & vocalis, thyro-epiglotticus, thyrohyoid, tibialis anterior, tibialis posterior, transverse arytenoid, transversospinalis-multifidus, transversospinalis-rotatores, transversospinalis-semispinalis, transversus abdominis, transversus thoracis, trapezius, triceps, vastus intermedius, vastus lateralis, vastus medialis, zygomaticus major, or zygomaticus minor. In some embodiments, the cell is a myocyte. In some embodiments, the cell is a muscle cell. In some embodiments, the muscle cell is a skeletal muscle cell. In some embodiments, the skeletal muscle cell is a red (slow) skeletal muscle cell, a white (fast) skeletal muscle cell or an intermediate skeletal muscle cell.

In some aspects, disclosed herein are modified cells or populations of modified cells, wherein the modified cell comprises an effector protein described herein, a nucleic acid encoding an effector protein described herein, or a combination thereof. In some embodiments, the modified cell comprises a fusion effector protein described herein, a nucleic acid encoding an effector protein described herein, or a combination thereof. In some embodiments, the modified cell is a modified prokaryotic cell. In some embodiments, the modified cell is a modified eukaryotic cell. A modified cell may be a modified fungal cell. In some embodiments, the modified cell is a modified vertebrate cell. In some embodiments, the modified cell is a modified invertebrate cell. In some embodiments, the modified cell is a modified mammalian cell. In some embodiments, the modified cell is a modified human cell. In some embodiments, the modified cell is in a subject. A modified cell may be in vitro. A modified cell may be in vivo. A modified cell may be ex vivo. A modified cell may be a cell in a cell culture. A modified cell may be a cell obtained from a biological fluid, organ, or tissue of a subject and modified with a composition, device, kit, system and/or method described herein. Non-limiting examples of biological fluids are blood, plasma, serum, and cerebrospinal fluid. Non-limiting examples of tissues and organs are bone marrow, adipose tissue, skeletal muscle, smooth muscle, spleen, thymus, brain, lymph node, adrenal gland, prostate gland, intestine, colon, liver, kidney, pancreas, heart, lung, bladder, ovary, uterus, breast, and testes. Non-limiting examples of cells that may be obtained from a subject are hepatocytes, epithelial cells, endothelial cells, neurons, cardiomyocytes, muscle cells and adipocytes. Non-limiting examples of cells that may be modified with compositions, devices, kits, systems and methods described herein include immune cells, such as CAR T-cells, T-cells, B-cells, NK cells, granulocytes, basophils, eosinophils, neutrophils, mast cells, monocytes, macrophages, dendritic cells, microglia, Kuppfer cells, antigen-presenting cells (APC), or adaptive cells.

Non-limiting examples of cells that may be engineered or modified with compositions, devices, kits, systems and methods described herein include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells. A cell may be a pluripotent cell.

Non-limiting examples of cells that may be engineered or modified with compositions, devices, kits, systems and methods described herein include plant cells, such as parenchyma, sclerenchyma, collenchyma, xylem, phloem, germline (e.g., pollen). Cells from lycophytes, ferns, gymnosperms, angiosperms, bryophytes, charophytes, chlorophytes, rhodophytes, or glaucophytes.

Methods may comprise cell line engineering. Generally, cell line engineering comprises modifying a pre-existing cell (e.g., naturally occurring or engineered) or pre-existing cell line to produce a novel cell line or modified cell line. In some embodiments, modifying the pre-existing cell or cell line comprises contacting the pre-existing cell or cell line with an effector protein or fusion effector protein described herein and a guide nucleic acid. The resulting modified cell line may be useful for production of a protein of interest. Non-limiting examples of cell lines includes: 132-d5 human fetal fibroblasts, 10.1 mouse fibroblasts, 293-T, 3T3, 3T3 Swiss, 3T3-L1, 721, 9L, A-549, A10, A172, A20, A253, A2780, A2780ADR, A2780cis, A375, A431, ALC, ARH-77, B16, B35, BALB/3T3 mouse embryo fibroblast, BC-3, BCP-1 cells, BEAS-2B, BHK-21, BR 293, BS-C-1 monkey kidney epithelial, Bcl-1, bEnd.3, BxPC3, C3H-10T1/2, C6/36, C8161, CCRF-CEM, CHO, CHO Dhfr−/−, CHO-7, CHO-IR, CHO-K1, CHO-K2, CHO-T, CIR, CML T1, CMT, COR-L23, COR-L23/5010, COR-L23/CPR, COR-L23/R23, COS, COS-1, COS-6, COS-7, COS-M6A, COV-434, CT26, CTLL-2, CV1, CaCo2, Cal-27, Calu1, D17, DH82, DLD2, DU145, DuCaP, EL4, EM2, EM3, EMT6/AR1, EMT6/AR10.0, FM3, H1299, H69, HASMC, HB54, HB55, HB56, HCA2, HEK-293, HEKa, HEKn, HL-60, HMEC, HT-29, HUVEC, HeLa, HeLa B, HeLa T4, HeLa-S3, Hep G2, Hepa1c1c7, Huh1, Huh4, Huh7, IC21, J45.01, J82, JY cells, Jurkat, Jurkat, K562 cells, KCL22, KG1, KYO1, Ku812, LNCap, LRMB, MC-38, MCF-10A, MCF-7, MDA-MB-231, MDA-MB-435, MDA-MB-468, MDCK II, MDCK II, MEF, mIMCD-3C8161, MOLT, MONO-MAC 6, MOR/0.2R, MRC5, MTD-1A, Ma-Mel 1-48, MiaPaCell, MyEnd, NALM-1, NCI-H69/CPR, NCI-H69/LX10, NCI-H69/LX20, NCI-H69/LX4, NHDF, NIH-3T3, NRK, NRK-52E, NW-145, OPCN/OPCT cell lines, P388D1, PC-3, PNT-1A/PNT 2, Panc1, Peer, RIN-5F, RMA/RMAS, RPTE, Rat6, Raw264.7, RenCa, SEM-K2, SK-UT, SKOV3, SW480, SW620, Saos-2 cells, Sf-9, SkBr3, T-47D, T2, T24, T84, TF1, THP1 cell line, TIB55, U373, U87, U937, VCaP, Vero cells, WEHI-231, WM39, WT-49, X63, YAC-1, and YAR.

Generating a genetically modified cell may comprise contacting a target cell with an effector protein or a fusion effector protein described herein and a guide nucleic acid. Contacting may comprise electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof. In some embodiments, the nanoparticle delivery comprises lipid nanoparticle delivery or gold nanoparticle delivery. In some embodiments, the nanoparticle delivery comprises lipid nanoparticle delivery. In some embodiments, the nanoparticle delivery comprises gold nanoparticle delivery.

Methods of editing described herein may be performed in a subject. In some embodiments, the methods comprise administering compositions described herein to the subject. In some embodiments, the subject is a human. In some embodiments, the subject is a mammal (e.g., rat, mouse, cow, dog, pig, sheep, horse). In some embodiments, the subject is a vertebrate or an invertebrate. In some embodiments, the subject is a laboratory animal. In some embodiments, the subject is a patient. In some embodiments, the subject is at risk of developing, suffering from, or displaying symptoms of a disease. In some embodiments, the subject may have a mutation associated with a gene described herein. In some embodiments, the subject may display symptoms associated with a mutation of a gene described herein.

### IX. Detection of Target Nucleic Acids

Described herein are devices, systems, fluidic devices, kits, and methods for detecting the presence of a target nucleic acid in a sample. In some embodiments, an effector protein-guide nucleic acid complex may comprise high selectivity for a target sequence. In some embodiments, an RNP comprise a selectivity of at least 200:1, 100:1, 50:1, 20:1, 10:1, or 5:1 for a target nucleic acid over a single nucleotide variant of the target nucleic acid. In some embodiments, an RNP may comprise a selectivity of at least 5:1 for a target nucleic acid over a single nucleotide variant of the target nucleic acid.

By leveraging such effector protein selectivity, some methods described herein may detect a target nucleic acid present in the sample in various concentrations or amounts as a target nucleic acid population. In some embodiments, the method detects at least 2 target nucleic acid populations. In some embodiments, the method detects at least 3, 4, 5, 6, 7, 8, 9, 10, 20, 30, 40, or 50 target nucleic acid populations. In some embodiments, the method detects 3 to 50, 5 to 40, or 10 to 25 target nucleic acid populations. In some embodiments, the method detects at least 2 individual target nucleic acids. In some embodiments, the method detects at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 individual target nucleic acids. In some embodiments, the method detects 1 to 10,000, 100 to 8000, 400 to 6000, 500 to 5000, 1000 to 4000, or 2000 to 3000 individual target nucleic acids. In some embodiments, the method detects target nucleic acid present at least at one copy per 10 non-target nucleic acids, 102 non-target nucleic acids, 103 non-target nucleic acids, 104 non-target nucleic acids, 105 non-target nucleic acids, 106 non-target nucleic acids, 107 non-target nucleic acids, 108 non-target nucleic acids, 109 non-target nucleic acids, or 1010 non-target nucleic acids.

In some embodiments, compositions described herein exhibit indiscriminate trans-cleavage of a nucleic acid (e.g., a ssDNA), enabling their use for detection of a nucleic acid (e.g., DNA) in samples. In some embodiments, target nucleic acids are generated from many nucleic acid templates (RNA) in order to achieve cleavage of a reporter (e.g., a FQ reporter) in a device (e.g., a DETECTR platform). Certain effector proteins may be activated by a nucleic acid (e.g., a ssDNA), upon which they may exhibit trans-cleavage of the nucleic acid (e.g., ssDNA) and may, thereby, be used to cleave the reporter molecules (e.g., ssDNA FQ reporter molecules) in a device (e.g., a DETECTR system). These effector proteins may target nucleic acids present in the sample or nucleic acids generated and/or amplified from any number of nucleic acid templates (e.g., RNA). Described herein are reagents comprising a single stranded reporter nucleic acid comprising a detection moiety, wherein the reporter nucleic acid (e.g., a ssDNA-FQ reporter described herein) is capable of being cleaved by the effector protein, upon generation (e.g., cDNA) and amplification of nucleic acids from a nucleic acid template (e.g., ssRNA) using the methods disclosed herein, thereby generating a first detectable signal. While DNA is used as an exemplary reporter in the foregoing, any suitable reporter may be used, including the exemplary reporters described in TABLE 12 herein.

In some embodiments, a target nucleic acid is an amplified nucleic acid of interest. In some embodiments, the nucleic acid of interest is any nucleic acid disclosed herein or from any sample as disclosed herein. In some embodiments, the nucleic acid of interest is DNA. In some embodiments, the nucleic acid of interest is an RNA. In some embodiments, the nucleic acid of interest is an RNA that is reverse transcribed before amplification. In some embodiments, the target nucleic acid is an amplicon of a target nucleic acid (DNA or RNA) generated via amplification (with or without reverse transcription). In some embodiments, the target nucleic acid is an amplicon of a target nucleic acid (DNA or RNA) generated via amplification that is reverse transcribed before amplification.

In some embodiments, target nucleic acids may activate an effector protein to initiate sequence-independent cleavage of a nucleic acid-based reporter (e.g., a reporter comprising a DNA sequence, or a reporter comprising DNA and RNA). For example, an effector protein of the present disclosure is activated by a target nucleic acid to cleave reporters having a DNA (also referred to herein as a “DNA reporter”). The DNA reporter may comprise a single-stranded DNA labelled with a detection moiety or may be any DNA reporter as disclosed herein.

### Samples

Various sample types comprising a target nucleic acid of interest are consistent with the present disclosure. These samples may comprise a target nucleic acid for detection. In some embodiments, the detection of the target nucleic indicates an ailment, such as a disease, cancer, or genetic disorder, or genetic information, such as for phenotyping, genotyping, or determining ancestry and are compatible with the reagents and support mediums as described herein. Generally, a sample from an individual or an animal or an environmental sample may be obtained to test for presence of a disease, cancer, genetic disorder, or any mutation of interest.

In some embodiments, a sample comprises a target nucleic acid from 0.05% to 20% of total nucleic acids in the sample. In some embodiments, the target nucleic acid is 0.1% to 10% of the total nucleic acids in the sample. In some embodiments, the target nucleic acid is 0.1% to 5% of the total nucleic acids in the sample. In some embodiments, the target nucleic acid is 0.1% to 1% of the total nucleic acids in the sample. In some embodiments, the target nucleic acid is in any amount less than 100% of the total nucleic acids in the sample. In some embodiments, the target nucleic acid is 100% of the total nucleic acids in the sample. In some embodiments, the sample comprises a portion of the target nucleic acid and at least one nucleic acid comprising less than 100% sequence identity to the portion of the target nucleic acid but no less than 50% sequence identity to the portion of the target nucleic acid. For example, the portion of the target nucleic acid comprises a mutation as compared to at least one nucleic acid comprising less than 100% sequence identity to the portion of the target nucleic acid but no less than 50% sequence identity to the portion of the target nucleic acid. In some embodiments, the portion of the target nucleic acid comprises a single nucleotide mutation as compared to at least one nucleic acid comprising less than 100% sequence identity to the portion of the target nucleic acid but no less than 50% sequence identity to the portion of the target nucleic acid.

In some embodiments, a sample comprises target nucleic acid populations at different concentrations or amounts. In some embodiments, the sample has at least 2 target nucleic acid populations. In some embodiments, the sample has at least 3, 4, 5, 6, 7, 8, 9, 10, 20, 30, 40, or 50 target nucleic acid populations. In some embodiments, the sample has 3 to 50, 5 to 40, or 10 to 25 target nucleic acid populations.

In some embodiments, a sample has at least 2 individual target nucleic acids. In some embodiments, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 individual target nucleic acids. In some embodiments, the sample comprises 1 to 10,000, 100 to 8000, 400 to 6000, 500 to 5000, 1000 to 4000, or 2000 to 3000 individual target nucleic acids.

In some embodiments, a sample comprises one copy of target nucleic acid per 10 non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

In some embodiments, samples comprise a target nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 μM, less than 7 μM, less than 8 μM, less than 9 μM, less than 10 μM, less than 100 μM, or less than 1 mM. In some embodiments, the sample comprises a target nucleic acid at a concentration of 1 nM to 2 nM, 2 nM to 3 nM, 3 nM to 4 nM, 4 nM to 5 nM, 5 nM to 6 nM, 6 nM to 7 nM, 7 nM to 8 nM, 8 nM to 9 nM, 9 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1 μM, 1 μM to 2 μM, 2 μM to 3 μM, 3 μM to 4 μM, 4 μM to 5 μM, 5 μM to 6 μM, 6 μM to 7 μM, 7 μM to 8μ, 8 μM to 9μ, 9 μM to 10μ, 10 μM to 100 μM, 100 μM to 1 mM, 1 nM to 10 nM, 1 nM to 100 nM, 1 nM to 1 μM, 1 nM to 10 μM, 1 nM to 100 μM, 1 nM to 1 mM, 10 nM to 100 nM, 10 nM to 1 μM, 10 nM to 10 μM, 10 nM to 100 μM, 10 nM to 1 mM, 100 nM to 1 μM, 100 nM to 10 μM, 100 nM to 100 μM, 100 nM to 1 mM, 1 μM to 10 μM, 1 μM to 100 μM, 1 μM to 1 mM, 10 μM to 100 μM, 10 μM to 1 mM, or 100 μM to 1 mM. In some embodiments, the sample comprises a target nucleic acid at a concentration of 20 nM to 200 μM, 50 nM to 100 μM, 200 nM to 50 μM, 500 nM to 20 μM, or 2 μM to 10 μM. In some embodiments, the target nucleic acid is not present in the sample.

In some embodiments, samples comprise fewer than 10 copies, fewer than 100 copies, fewer than 1000 copies, fewer than 10,000 copies, fewer than 100,000 copies, or fewer than 1,000,000 copies of a target nucleic acid. In some embodiments, the sample comprises 10 copies to 100 copies, 100 copies to 1000 copies, 1000 copies to 10,000 copies, 10,000 copies to 100,000 copies, 100,000 copies to 1,000,000 copies, 10 copies to 1000 copies, 10 copies to 10,000 copies, 10 copies to 100,000 copies, 10 copies to 1,000,000 copies, 100 copies to 10,000 copies, 100 copies to 100,000 copies, 100 copies to 1,000,000 copies, 1,000 copies to 100,000 copies, or 1,000 copies to 1,000,000 copies of a target nucleic acid. In some embodiments, the sample comprises 10 copies to 500,000 copies, 200 copies to 200,000 copies, 500 copies to 100,000 copies, 1000 copies to 50,000 copies, 2000 copies to 20,000 copies, 3000 copies to 10,000 copies, or 4000 copies to 8000 copies. In some embodiments, the target nucleic acid is not present in the sample.

In some embodiments, the sample is a biological sample, an environmental sample, or a combination thereof. Non-limiting examples of biological samples are blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, and a tissue sample (e.g., a biopsy sample). A tissue sample from a subject may be dissociated or liquified prior to application to detection system of the present disclosure. Non-limiting examples of environmental samples are soil, air, or water. In some embodiments, an environmental sample is taken as a swab from a surface of interest or taken directly from the surface of interest.

In some embodiments, the sample is a raw (unprocessed, unedited, unmodified) sample. Raw samples may be applied to a system for detecting or editing a target nucleic acid, such as those described herein. In some embodiments, the sample is diluted with a buffer or a fluid or concentrated prior to its application to the system or be applied neat to the detection system. Sometimes, the sample contains no more 20 μl of buffer or fluid. The sample, in some embodiments, is contained in no more than 0.01, 0.1, 0.5, 1, 5, 10, 15, 20, 25, 30, 35 40, 45, 50, 55, 60, 65, 70, 75, 80, 90, 100, 200, 300, 400, 500 μl, or any of value 0.01 μl to 500 μl, 0.1 μL to 100 μL, or more preferably 1 μL to 50 μL of buffer or fluid. Sometimes, the sample is contained in more than 500 μl. In some embodiments, the systems, devices, kits, and methods disclosed herein are compatible with the buffers or fluid disclosed herein.

In some embodiments, the sample is taken from a single-cell eukaryotic organism; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some embodiments, the sample is taken from nematodes, protozoans, helminths, or malarial parasites. In some embodiments, the sample comprises nucleic acids from a cell lysate from a eukaryotic cell, a mammalian cell, a human cell, a prokaryotic cell, or a plant cell. In some embodiments, the sample comprises nucleic acids expressed from a cell.

In some embodiments, samples are used for diagnosing a disease. In some embodiments the disease is cancer. The sample used for cancer testing may comprise at least one target nucleic acid that may hybridize to a guide nucleic acid of the reagents described herein. The target nucleic acid, in some embodiments, comprises a portion of a gene comprising a mutation associated with a disease, such as cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker. In some embodiments, the assay may be used to detect “hotspots” in target nucleic acids that may be predictive of a cancer. In some embodiments, the target nucleic acid comprises a portion of a nucleic acid that is associated with a cancer. In some embodiments, the target nucleic acid is a portion of a nucleic acid from a genomic locus, any DNA amplicon of, a reverse transcribed mRNA, or a cDNA from a locus of at least one of a gene set forth in TABLE 7. Any region of the aforementioned gene loci may be probed for a mutation or deletion using the compositions and methods disclosed herein. For example, in the EGFR gene locus, the compositions and methods for detection disclosed herein may be used to detect a single nucleotide polymorphism or a deletion.

In some embodiments, samples are used to diagnose a genetic disorder, also referred to as genetic disorder testing. The sample used for genetic disorder testing may comprise at least one target nucleic acid that may hybridize to a guide nucleic acid of the reagents described herein. The target nucleic acid, in some embodiments, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some embodiments, the target nucleic acid is a nucleic acid from a genomic locus, a transcribed mRNA, or a reverse transcribed mRNA, a DNA amplicon of or a cDNA from a locus of at least one of a gene set forth in TABLE 7.

A sample used for phenotyping testing may comprise at least one target nucleic acid that may hybridize to a guide nucleic acid of the reagents described herein. The target nucleic acid, in some embodiments, is a nucleic acid encoding a sequence associated with a phenotypic trait. A sample used for genotyping testing may comprise at least one target nucleic acid that may hybridize to a guide nucleic acid of the reagents described herein. A target nucleic acid, in some embodiments, is a nucleic acid encoding a sequence associated with a genotype of interest. A sample used for ancestral testing may comprise at least one target nucleic acid that may hybridize to a guide nucleic acid of the reagents described herein. A target nucleic acid, in some embodiments, is a nucleic acid encoding a sequence associated with a geographic region of origin or ethnic group. A sample may be used for identifying a disease status. For example, a sample is any sample described herein, and is obtained from a subject for use in identifying a disease status of a subject. In some embodiments, the disease is cancer. In some embodiments, the disease is a genetic disorder. In some embodiments, a method comprises obtaining a serum sample from a subject; and identifying a disease status of the subject.

### Systems for Detection

Disclosed herein, in some aspects, are systems for detecting a target nucleic acid, comprising any one of the effector proteins described herein. In some embodiments, systems comprise a guide nucleic acid. Systems may be used to detect a target nucleic acid. In some embodiments, systems comprise an effector protein described herein, one or more guide nucleic acids, a reagent, support medium, or a combination thereof.

In some embodiments, systems comprise a fusion protein described herein. In some embodiments, effector proteins comprise an amino acid sequence that is at least 70%, at least 75%, at least 80%, at least 85%, at least 90%, at least 95%, or 100% identical to any one of the amino acid sequences selected from TABLE 1, e.g., any one of the sequences set forth in SEQ ID NOS: 1-28, or in SEQ ID NOS: 93-142. In some embodiments, the amino acid sequence of the effector protein is at least 70%, at least 75%, at least 80%, at least 85%, at least 90%, at least 95%, or 100% identical to any one of the amino acid sequences selected from TABLE 1, e.g., any one of the sequences set forth in SEQ ID NOS: 1-28, or in SEQ ID NOS: 93-142.

In some embodiments, systems, compositions, methods, kits, devices, and solutions comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more, effector proteins, or nucleic acids encoding the effector proteins, as described herein. In some embodiments, systems, compositions, methods, kits, devices, and solutions comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more, effector proteins, or nucleic acids encoding the effector proteins, as described herein. In some embodiments, systems, compositions, methods, kits, devices, and solutions comprise 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 M, 450 μM, 500 μM, or more, effector proteins, or nucleic acids encoding the effector proteins, as described herein. In some embodiments, systems, compositions, methods, kits, devices, and solutions comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more, effector proteins, or nucleic acids encoding the effector proteins, as described herein.

In some embodiments, systems, compositions, methods, kits, devices, and solutions comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more, guide nucleic acids, or nucleic acids encoding the guide nucleic acids, as described herein. In some embodiments, systems, compositions, methods, kits, devices, and solutions comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more, guide nucleic acids, or nucleic acids encoding the guide nucleic acids, as described herein. In some embodiments, systems, compositions, methods, kits, devices, and solutions comprise 1 μM, 2 μM, 3 μM, 4 μM, 5 μM, 6 μM, 7 μM, 8 μM, 9 μM, 10 μM, 20 M, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM, or more, guide nucleic acids, or nucleic acids encoding the guide nucleic acids, as described herein. In some embodiments, systems, compositions, methods, kits, devices, and solutions comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more, guide nucleic acids, or nucleic acids encoding the guide nucleic acids, as described herein.

Systems may be used for detecting the presence of a target nucleic acid associated with or causative of a disease, such as cancer, a genetic disorder, or an infection. In some embodiments, systems are useful for phenotyping, genotyping, or determining ancestry. Unless specified otherwise, systems include kits and may be referred to as kits. Unless specified otherwise, systems include devices and may also be referred to as devices. Systems described herein may be provided in the form of a companion diagnostic assay or device, a point-of-care assay or device, or an over-the-counter diagnostic assay/device.

Reagents and effector proteins of various systems may be provided in a reagent chamber or on a support medium. Alternatively, the reagent and/or effector protein may be contacted with the reagent chamber or the support medium by the individual using the system. An exemplary reagent chamber is a test well or container. The opening of the reagent chamber may be large enough to accommodate the support medium. Optionally, the system comprises a buffer and a dropper. The buffer may be provided in a dropper bottle for ease of dispensing. The dropper may be disposable and transfer a fixed volume. The dropper may be used to place a sample into the reagent chamber or on the support medium.

### System Solutions

In general, systems comprise a solution in which the activity of an effector protein occurs. Often, the solution comprises or consists essentially of a buffer. The solution or buffer may comprise a buffering agent, a salt, a crowding agent, a detergent, a reducing agent, a competitor, or a combination thereof. Often the buffer is the primary component or the basis for the solution in which the activity occurs. Thus, concentrations for components of buffers described herein (e.g., buffering agents, salts, crowding agents, detergents, reducing agents, and competitors) are the same or essentially the same as the concentration of these components in the solution in which the activity occurs. In some embodiments, a buffer is required for cell lysis activity or viral lysis activity.

In some embodiments, systems comprise a buffer, wherein the buffer comprise at least one buffering agent. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, IB1, TCEP, EGTA, Tween 20, KCl, KOH, MgCl2, glycerol, or any combination thereof. In some instances, a buffer may comprise Tris-HCl pH 8.8, VLB, EGTA, CH3COOH, TCEP, IsoAmp®, (NH4)2SO4, KCl, MgSO4, Tween20, KOAc, MgOAc, BSA, phosphate, citrate, acetate, imidazole, or any combination thereof. In some embodiments, the concentration of the buffering agent in the buffer is 1 mM to 200 mM. A buffer compatible with an effector protein may comprise a buffering agent at a concentration of 10 mM to 30 mM. A buffer compatible with an effector protein may comprise a buffering agent at a concentration of about 20 mM. A buffering agent may provide a pH for the buffer or the solution in which the activity of the effector protein occurs. The pH may be 3 to 4, 3.5 to 4.5, 4 to 5, 4.5 to 5.5, 5 to 6, 5.5 to 6.5, 6 to 7, 6.5 to 7.5, 7 to 8, 7.5 to 8.5, 8 to 9, 8.5 to 9.5, 9 to 10, or 9.5 to 10.5.

In some embodiments, systems comprise a solution, wherein the solution comprises one or more salt. Accordingly, in some embodiments, the salt may be one or more salt selected from a magnesium salt, a zinc salt, a potassium salt, a calcium salt, and a sodium salt. In some embodiments, the salt is a combination of two or more salts. For example, in some embodiments, the salt is a combination of two or more salts selected from a magnesium salt, a zinc salt, a potassium salt, a calcium salt and a sodium salt. In some embodiments, the salt is magnesium acetate. In some embodiments, the salt is magnesium chloride. In some embodiments, the salt is potassium acetate. In some embodiments, the salt is potassium nitrate. In some embodiments, the salt is zinc chloride. In embodiments, the salt is sodium chloride. In some embodiments, the salt is potassium chloride. In some embodiments, the concentration of the one or more salt in the solution is about 0.001 mM to about 500 mM. In some embodiments, the concentration of the salt is about 0.001 mM to about 400 mM. In some embodiments, the concentration of the salt is about 0.001 mM to about 300 mM. In some embodiments, the concentration of the salt is about 0.001 mM to about 200 mM. In some embodiments, the concentration of the salt is about 0.001 mM to about 100 mM. In some embodiments, the concentration of the salt is about 0.001 mM to about 10 mM. In some embodiments, the concentration of the salt is about 0.01 mM to about 500 mM. In some embodiments, the concentration of the salt is about 0.01 mM to about 400 mM. In some embodiments, the concentration of the salt is about 0.01 mM to about 300 mM. In some embodiments, the concentration of the salt is about 0.01 mM to about 200 mM. In some embodiments, the concentration of the salt is about 0.01 mM to about 100 mM. In some embodiments, the concentration of the salt is about 0.01 mM to about 10 mM. In some embodiments, the concentration of the salt is about 0.1 mM to about 500 mM. In some embodiments, the concentration of the salt is about 0.1 mM to about 400 mM. In some embodiments, the concentration of the salt is about 0.1 mM to about 300 mM. In some embodiments, the concentration of the salt is about 0.1 mM to about 200 mM. In some embodiments, the concentration of the salt is about 0.1 mM to about 100 mM. In some embodiments, the concentration of the salt is about 0.1 mM to about 10 mM. In some embodiments, the concentration of the salt is about 1 mM to about 500 mM. In some embodiments, the concentration of the salt is about 1 mM to about 400 mM. In some embodiments, the concentration of the salt is about 1 mM to about 300 mM. In some embodiments, the concentration of the salt is about 1 mM to about 200 mM. In some embodiments, the concentration of the salt is about 1 mM to about 100 mM. In some embodiments, the concentration of the salt is about 1 mM to about 10 mM. In some embodiments, the concentration of the salt is about 10 mM to about 500 mM. In some embodiments, the concentration of the salt is about 10 mM to about 400 mM. In some embodiments, the concentration of the salt is about 10 mM to about 300 mM. In some embodiments, the concentration of the salt is about 10 mM to about 200 mM. In some embodiments, the concentration of the salt is about 10 mM to about 100 mM. In some embodiments, the concentration of the salt is about 100 mM to about 500 mM. In some embodiments, the concentration of the salt is about 100 mM to about 400 mM. In some embodiments, the concentration of the salt is about 100 mM to about 300 mM. In some embodiments, the concentration of the salt is about 100 mM to about 200 mM. In some embodiments, the salt is potassium acetate and the concentration of salt in the solution is about 100 mM. In some embodiments, the salt is potassium acetate or sodium chloride and the concentration of salt in the solution is about 200 mM. In some embodiments, the salt is potassium acetate or sodium chloride and the concentration of salt in the solution is about 100 mM to about 200 mM.

In some embodiments, systems comprise a solution, wherein the solution comprises at least one crowding agent. A crowding agent may reduce the volume of solvent available for other molecules in the solution, thereby increasing the effective concentrations of said molecules. Exemplary crowding agents include glycerol and bovine serum albumin. In some embodiments, the crowding agent is glycerol. In some embodiments, the concentration of the crowding agent in the solution is 0.01% (v/v) to 10% (v/v). In some embodiments, the concentration of the crowding agent in the solution is 0.5% (v/v) to 10% (v/v).

In some embodiments, systems comprise a solution, wherein the solution comprises at least one detergent. Exemplary detergents include Tween, Triton-X, and IGEPAL. A solution may comprise Tween, Triton-X, or any combination thereof. A solution may comprise Triton-X. A solution may comprise IGEPAL CA-630. In some embodiments, the concentration of the detergent in the solution is 2% (v/v) or less. In some embodiments, the concentration of the detergent in the solution is 1% (v/v) or less. In some embodiments, the concentration of the detergent in the solution is 0.00001% (v/v) to 0.01% (v/v). In some embodiments, the concentration of the detergent in the solution is about 0.01% (v/v).

In some embodiments, systems comprise a solution, wherein the solution comprises at least one reducing agent. Exemplary reducing agents comprise dithiothreitol (DTT), β-mercaptoethanol (BME), or tris(2-carboxyethyl) phosphine (TCEP). In some embodiments, the reducing agent is DTT. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is 0.5 mM to 2 mM. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is about 1 mM.

In some embodiments, systems comprise a solution, wherein the solution comprises a competitor. In general, competitors compete with the target nucleic acid or the reporter nucleic acid for cleavage by the effector protein or a dimer thereof. Exemplary competitors include heparin, and imidazole, and salmon sperm DNA. In some embodiments, the concentration of the competitor in the solution is 1 μg/mL to 100 μg/mL. In some embodiments, the concentration of the competitor in the solution is 40 μg/mL to 60 μg/mL.

In some embodiments, systems comprise a solution, wherein the solution comprises a co-factor. In some embodiments, the co-factor allows an effector protein or a multimeric complex thereof to perform a function, including pre-crRNA processing and/or target nucleic acid cleavage. The suitability of a cofactor for an effector protein or a multimeric complex thereof may be assessed, such as by methods based on those described by Sundaresan et al. (Cell Rep. 2017 Dec. 26; 21(13): 3728-3739). In some embodiments, an effector or a multimeric complex thereof forms a complex with a co-factor. In some embodiments, the co-factor is a divalent metal ion. In some embodiments, the divalent metal ion is selected from Mg2+, Mn2+, Zn2+, Ca2+, Cu2+. In some embodiments, the divalent metal ion is Mg2+. In some embodiments, the co-factor is Mg2+.

In some embodiments, systems, and compositions for use with systems comprise a catalytic reagent for signal improvement or enhancement. In some embodiments, the catalytic reagent enhances signal generation via hydrolysis of inorganic pyrophosphates. In some embodiments, catalytic reagents enhance signal generation via enhancement of DNA replication. In some embodiments, catalytic reagents enhance signal amplification via revival of Mg2+ ions in the buffer solution which may otherwise be taken up by the phosphates produced from usage of dNTPs during the LAMP reaction. In some embodiments, catalytic reagents enhance signal generation by reviving the concentration of Mg2+ ions in the buffer thereby enhancing the function of an effector protein. In some embodiments, the catalytic reagent for signal improvement may be an enzyme. In some embodiments, the catalytic reagent for signal improvement may be a Thermostable Inorganic Pyrophosphatase (TIPP). In some embodiments, the catalytic reagent for signal improvement are particularly useful in amplification and/or detection reactions as described herein. Other exemplary reagents useful for amplification and/or detection reactions (i.e., amplification and detection reagents, respectively) are described throughout herein.

In some embodiments, systems comprise TIPP. TIPP may be useful for enhancing a detectable signal relative to a system that does not comprise TIPP. Any of the systems, methods, or compositions described herein may comprise TIPP or the use thereof. In some embodiments, compositions useful for a system disclosed herein comprise TIPP. In some embodiments, compositions comprise about 0.5 enzyme unit (U) TIPP per 10 μL of solution. In some embodiments, compositions comprise at least about 0.1 U TIPP per 10 μL of solution. In some embodiments, compositions comprise at most about 2 U TIPP per 10 μL of solution. In some embodiments, compositions comprise at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 U TIPP per 10 μL of solution. In some embodiments, compositions comprise at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 U TIPP per 10 μL of solution. In some embodiments, TIPP is present in a composition at a concentration of 0.125 Units, 0.5 Units, 0.25 Units, 1.0 Units, 2.0 Units, 2.5 Units, or 4 Units per discrete reaction volume. In some embodiments, TIPP is provided in a system separately from a buffer provided in the system. In some embodiments, systems comprise a buffer, wherein TIPP is provided in the buffer.

In some embodiments, TIPP improves the signal to noise ratio of an effector protein-based detection reaction. In some embodiments, TIPP improves overall signal (e.g., fluorescence of a cleaved reporter. TIPP may improve signal by a factor, wherein the signal is indicative of the presence of a target nucleic acid. In some embodiments, the factor may be at least about 1.1, at least about 2, at least about 3, at least about 4, at least about 5, at least about 6, at least about 7, at least about 8, at least about 9, or at least about 10.

Also provided herein are reagents for: detection reactions, nuclease purification, cell lysis, in vitro transcription reactions, amplification reactions, reverse transcription reactions, and the like. In some embodiments, systems, compositions, and/or solutions described herein comprise one or more of: detection reagents, nuclease purification reagents, cell lysis reagents, in vitro transcription reagents, amplification reagents, reverse transcription reagents, and combinations thereof. In some embodiments, any such reagents suitable with the compositions, methods, systems, devices, and/or kits described herein may be used to achieve one or more of the foregoing described reactions. Reagents provided herein may be used with any other solution components described herein, including buffers, amino acids or derivatives thereof, chaotropes, chelators, cyclodextrins, inhibitors, ionic liquids, linkers, metals, non-detergent sulfobetaines, organic acids, osmolytes, peptides, polyamides, polymers, polyols, polyols and salts, salts, or combinations thereof.

### Detection Reagents/Components and Reporters

In some embodiments, systems disclosed herein comprise detection reagents to facilitate detection of nucleic acids as described herein. In some embodiments, the detection reagent is operably linked to an effector protein described herein such that a detection event occurs upon contacting the detection reagent and effector protein with a target nucleic acid. Upon the occurrence of the detection event, a signal (e.g., a detectable signal or detectable product) can be generated thereby indicating detection of the target nucleic acid. Any suitable detection reagent may be used, including: a nucleic acid (which may be referred to herein as a detection or reporter nucleic acid), a detection moiety, an additional polypeptide, or a combination thereof. Other detection reagents include buffers, reverse transcriptase mix, TIPP, a stain, and the like. Any reagents suitable with the detection reactions, events, and signals described herein are useful as detection reagents for the systems, compositions, methods, kits, devices, and solutions provided herein, including a buffer, stain (e.g., SYTO9), TIPP (e.g., 0.2 U), a reporter (e.g., a C12 FQ reporter) and the like.

In some embodiments, systems, compositions, methods, kits, devices, and solutions comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more of each detection reagent as described herein. In some embodiments, systems, compositions, methods, kits, devices, and solutions comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more of each detection reagent as described herein. In some embodiments, systems, compositions, methods, kits, devices, and solutions comprise 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM, or more of each detection reagent as described herein. In some embodiments, systems, compositions, methods, kits, devices, and solutions comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more of each detection reagent as described herein.

In some embodiments, systems disclosed herein comprise a reporter. By way of non-limiting and illustrative example, a reporter may comprise a single stranded nucleic acid and a detection moiety (e.g., a labeled single stranded RNA reporter), wherein the nucleic acid is capable of being cleaved by an effector protein (e.g., a CRISPR/Cas protein as disclosed herein) or a multimeric complex thereof, releasing the detection moiety, and generating a detectable signal. Cleavage of a reporter (e.g., a protein-nucleic acid) can produce a signal. The signal can indicate a presence of the target nucleic acid in the sample, and an absence of the signal can indicate an absence of the target nucleic acid in the sample. In some cases, cleavage of the protein-nucleic acid can produce a calorimetric signal, a potentiometric signal, an amperometric signal, an optical signal, or a piezo-electric signal. Various devices and/or sensors can be used to detect these different types of signals, which indicate whether a target nucleic acid is present in the sample. The sensors usable to detect such signals can include, for example, optical sensors (e.g., imaging devices for detecting fluorescence or optical signals with various wavelengths and frequencies), electric potential sensors, surface plasmon resonance (SPR) sensors, interferometric sensors, or any other type of sensor suitable for detecting calorimetric signals, potentiometric signals, amperometric signals, optical signals, or piezo-electric signals.

As used herein, “reporter” is used interchangeably with “reporter nucleic acid” or “reporter molecule”. The effector proteins disclosed herein, activated upon hybridization of a guide nucleic acid to a target nucleic acid, may cleave the reporter. Cleaving the “reporter” may be referred to herein as cleaving the “reporter nucleic acid,” the “reporter molecule,” or the “nucleic acid of the reporter.” Reporters may comprise RNA. Reporters may comprise DNA. Reporters may be double-stranded. Reporters may be single-stranded.

In some embodiments, reporters comprise a protein capable of generating a signal. A signal may be a calorimetric, potentiometric, amperometric, optical (e.g., fluorescent, colorimetric, etc.), or piezo-electric signal. In some embodiments, the reporter comprises a detection moiety. Suitable detectable labels and/or moieties that may provide a signal include, but are not limited to, an enzyme, a radioisotope, a member of a specific binding pair; a fluorophore; a fluorescent protein; a quantum dot; and the like.

In some embodiments, the reporter comprises a detection moiety and a quenching moiety. In some embodiments, the reporter comprises a cleavage site, wherein the detection moiety is located at a first site on the reporter and the quenching moiety is located at a second site on the reporter, wherein the first site and the second site are separated by the cleavage site. Sometimes the quenching moiety is a fluorescence quenching moiety. In some embodiments, the quenching moiety is 5′ to the cleavage site and the detection moiety is 3′ to the cleavage site. In some embodiments, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some embodiments, the detection moiety is at the 5′ terminus of the nucleic acid of a reporter. In some embodiments, the quenching moiety is at the 3′ terminus of the nucleic acid of a reporter.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, Ypet, mKO, HcRed, t-HcRed, DsRed, DsRed2, DsRed-monomer, J-Red, dimer2, t-dimer2 (12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, and glucose oxidase (GO).

In some embodiments, the detection moiety comprises an invertase. The substrate of the invertase may be sucrose. A DNS reagent may be included in the system to produce a colorimetric change when the invertase converts sucrose to glucose. In some embodiments, the reporter nucleic acid and invertase are conjugated using a heterobifunctional linker via sulfo-SMCC chemistry.

Suitable fluorophores may provide a detectable fluorescence signal in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Non-limiting examples of fluorophores are fluorescein amidite, 6-Fluorescein, IRDye 700, TYE 665, Alex Fluor 594, or ATTO TM 633 (NHS Ester). The fluorophore may be an infrared fluorophore. The fluorophore may emit fluorescence in the range of 500 nm and 720 nm. In some embodiments, the fluorophore emits fluorescence at a wavelength of 700 nm or higher. In other embodiments, the fluorophore emits fluorescence at about 665 nm. In some embodiments, the fluorophore emits fluorescence in the range of 500 nm to 520 nm, 500 nm to 540 nm, 500 nm to 590 nm, 590 nm to 600 nm, 600 nm to 610 nm, 610 nm to 620 nm, 620 nm to 630 nm, 630 nm to 640 nm, 640 nm to 650 nm, 650 nm to 660 nm, 660 nm to 670 nm, 670 nm to 680 nm, 690 nm to 690 nm, 690 nm to 700 nm, 700 nm to 710 nm, 710 nm to 720 nm, or 720 nm to 730 nm. In some embodiments, the fluorophore emits fluorescence in the range 450 nm to 750 nm, 500 nm to 650 nm, or 550 to 650 nm.

Systems may comprise a quenching moiety. A quenching moiety may be chosen based on its ability to quench the detection moiety. A quenching moiety may be a non-fluorescent fluorescence quencher. A quenching moiety may quench a detection moiety that emits fluorescence in the range of 500 nm and 720 nm. A quenching moiety may quench a detection moiety that emits fluorescence in the range of 500 nm and 720 nm. In some embodiments, the quenching moiety quenches a detection moiety that emits fluorescence at a wavelength of 700 nm or higher. In other embodiments, the quenching moiety quenches a detection moiety that emits fluorescence at about 660 nm or about 670 nm. In some embodiments, the quenching moiety quenches a detection moiety that emits fluorescence in the range of 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some embodiments, the quenching moiety quenches a detection moiety that emits fluorescence in the range 450 nm to 750 nm, 500 nm to 650 nm, or 550 to 650 nm. A quenching moiety may quench fluorescein amidite, 6-Fluorescein, IRDye 700, TYE 665, Alex Fluor 594, or ATTO TM 633 (NHS Ester). A quenching moiety may be Iowa Black RQ, Iowa Black FQ or IRDye QC-1 Quencher. A quenching moiety may quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety may be Iowa Black RQ (Integrated DNA Technologies), Iowa Black FQ (Integrated DNA Technologies) or IRDye QC-1 Quencher (LiCor). Any of the quenching moieties described herein may be from any commercially available source, may be an alternative with a similar function, a generic, or a non-tradename of the quenching moieties listed.

The generation of the detectable signal from the release of the detection moiety may indicate that cleavage by the effector protein has occurred and that the sample contains the target nucleic acid. In some embodiments, the detection moiety comprises a fluorescent dye. Sometimes the detection moiety comprises a fluorescence resonance energy transfer (FRET) pair. In some embodiments, the detection moiety comprises an infrared (IR) dye. In some embodiments, the detection moiety comprises an ultraviolet (UV) dye. Alternatively, or in combination, the detection moiety comprises a protein. Sometimes the detection moiety comprises a biotin. Sometimes the detection moiety comprises at least one of avidin or streptavidin. In some embodiments, the detection moiety comprises a polysaccharide, a polymer, or a nanoparticle. In some embodiments, the detection moiety comprises a gold nanoparticle or a latex nanoparticle.

A detection moiety may be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal may be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter.

The detectable signal may be a colorimetric signal or a signal visible by eye. In some embodiments, the detectable signal may be fluorescent, electrical, chemical, electrochemical, or magnetic. In some embodiments, the first detection signal may be generated by binding of the detection moiety to the capture molecule in the detection region, where the first detection signal indicates that the sample contained the target nucleic acid. Sometimes systems are capable of detecting more than one type of target nucleic acid, wherein the system comprises more than one type of guide nucleic acid and more than one type of reporter nucleic acid. In some embodiments, the detectable signal may be generated directly by the cleavage event. Alternatively, or in combination, the detectable signal may be generated indirectly by the signal event. Sometimes the detectable signal is not a fluorescent signal. In some embodiments, the detectable signal may be a colorimetric or color-based signal. In some embodiments, the detected target nucleic acid may be identified based on its spatial location on the detection region of the support medium. In some embodiments, the second detectable signal may be generated in a spatially distinct location than the first generated signal.

In some embodiments, the reporter nucleic acid is a single-stranded nucleic acid sequence comprising ribonucleotides. The nucleic acid of a reporter may be a single-stranded nucleic acid sequence comprising at least one ribonucleotide. In some embodiments, the nucleic acid of a reporter is a single-stranded nucleic acid comprising at least one ribonucleotide residue at an internal position that functions as a cleavage site. In some embodiments, the nucleic acid of a reporter comprises at least 2, at least 3, at least 4, at least 5, at least 6, at least 7, at least 8, at least 9, or at least 10 ribonucleotide residues at an internal position. In some embodiments, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some embodiments, the nucleic acid of a reporter has only ribonucleotide residues. In some embodiments, the nucleic acid of a reporter has only deoxyribonucleotide residues. In some embodiments, the nucleic acid comprises nucleotides resistant to cleavage by the effector protein described herein. In some embodiments, the nucleic acid of a reporter comprises synthetic nucleotides. In some embodiments, the nucleic acid of a reporter comprises at least one ribonucleotide residue and at least one non-ribonucleotide residue.

In some embodiments, the nucleic acid of a reporter comprises at least one uracil ribonucleotide. In some embodiments, the nucleic acid of a reporter comprises at least two uracil ribonucleotides. Sometimes the nucleic acid of a reporter has only uracil ribonucleotides. In some embodiments, the nucleic acid of a reporter comprises at least one adenine ribonucleotide. In some embodiments, the nucleic acid of a reporter comprises at least two adenine ribonucleotides. In some embodiments, the nucleic acid of a reporter has only adenine ribonucleotides. In some embodiments, the nucleic acid of a reporter comprises at least one cytosine ribonucleotide. In some embodiments, the nucleic acid of a reporter comprises at least two cytosine ribonucleotides. In some embodiments, the nucleic acid of a reporter comprises at least one guanine ribonucleotide. In some embodiments, the nucleic acid of a reporter comprises at least two guanine ribonucleotides. In some embodiments, a nucleic acid of a reporter comprises a single unmodified ribonucleotide. In some embodiments, a nucleic acid of a reporter comprises only unmodified deoxyribonucleotides.

In some embodiments, the nucleic acid of a reporter is 5 to 20, 5 to 15, 5 to 10, 7 to 20, 7 to 15, or 7 to 10 nucleotides in length. In some embodiments, the nucleic acid of a reporter is 3 to 20, 4 to 10, 5 to 10, or 5 to 8 nucleotides in length. In some embodiments, the nucleic acid of a reporter is 5 to 12 nucleotides in length. In some embodiments, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some embodiments, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length.

In some embodiments, systems comprise a plurality of reporters. The plurality of reporters may comprise a plurality of signals. In some embodiments, systems comprise at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 reporters. In some embodiments, there are 2 to 50, 3 to 40, 4 to 30, 5 to 20, or 6 to 10 different reporters.

In some embodiments, systems comprise an effector protein and a reporter nucleic acid configured to undergo trans cleavage by the effector protein. Trans cleavage of the reporter may generate a signal from the reporter or alter a signal from the reporter. In some embodiments, the signal is an optical signal, such as a fluorescence signal or absorbance band. Trans cleavage of the reporter may alter the wavelength, intensity, or polarization of the optical signal. For example, the reporter may comprise a fluorophore and a quencher, such that trans cleavage of the reporter separates the fluorophore and the quencher thereby increasing a fluorescence signal from the fluorophore. In some embodiments described herein is a method of assaying for a target nucleic acid in a sample comprising contacting the target nucleic acid with an effector protein, a non-naturally occurring guide nucleic acid that hybridizes to a segment of the target nucleic acid, and a reporter nucleic acid, and assaying for a change in a signal, wherein the change in the signal is produced by cleavage of the reporter nucleic acid.

In the presence of a large amount of non-target nucleic acids, an activity of an effector protein (e.g., an effector protein as disclosed herein) may be inhibited. This is because the activated effector proteins collaterally cleave all available nucleic acids. If total nucleic acids are present in large amounts, they may outcompete reporters for the effector proteins. In some embodiments, systems comprise an excess of reporter(s), such that when the system is operated and a solution of the system comprising the reporter is combined with a sample comprising a target nucleic acid, the concentration of the reporter in the combined solution-sample is greater than the concentration of the target nucleic acid. In some embodiments, the sample comprises amplified target nucleic acid. In some embodiments, the sample comprises an unamplified target nucleic acid. In some embodiments, the concentration of the reporter is greater than the concentration of target nucleic acids and non-target nucleic acids. The non-target nucleic acids may be from the original sample, either lysed or unlysed. The non-target nucleic acids may comprise byproducts of amplification. In some embodiments, systems comprise a reporter wherein the concentration of the reporter in a solution is at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids. In some embodiments, systems comprise a reporter wherein the concentration of the reporter in a solution is 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold excess of total nucleic acids.

Exemplary reporter nucleic acids are set forth in TABLE 12. In some embodiments, a reporter nucleic acid comprises a nucleotide sequence that is at least 65%, 70%, 75%, 80%, 85%, 90%, 95%, 97%, 99% or 100% identical to any one of the sequences as set forth in TABLE 12. In some embodiments, a reporter nucleic acid comprises a nucleotide sequence that is at least 65% identical to any one of the sequences as set forth in TABLE 12. In some embodiments, a reporter nucleic acid comprises a nucleotide sequence that is at least 70% identical to any one of the sequences as set forth in TABLE 12. In some embodiments, a reporter nucleic acid comprises a nucleotide sequence that is at least 75% identical to any one of the sequences as set forth in TABLE 12. In some embodiments, a reporter nucleic acid comprises a nucleotide sequence that is at least 80% identical to any one of the sequences as set forth in TABLE 12. In some embodiments, a reporter nucleic acid comprises a nucleotide sequence that is at least 85% identical to any one of the sequences as set forth in TABLE 12. In some embodiments, a reporter nucleic acid comprises a nucleotide sequence that is at least 90% identical to any one of the sequences as set forth in TABLE 12. In some embodiments, a reporter nucleic acid comprises a nucleotide sequence that is at least 95% identical to any one of the sequences as set forth in TABLE 12. In some embodiments, a reporter nucleic acid comprises a nucleotide sequence that is at least 97% identical to any one of the sequences as set forth in TABLE 12. In some embodiments, a reporter nucleic acid comprises a nucleotide sequence that is at least 99% identical to any one of the sequences as set forth in TABLE 12. In some embodiments, a reporter nucleic acid comprises a sequence that is identical to any one of the sequences as set forth in TABLE 12.

### Amplification Reagents/Components

In some embodiments, systems described herein comprise a reagent or component for amplifying a nucleic acid. Non-limiting examples of reagents for amplifying a nucleic acid include polymerases, primers, and nucleotides. In some embodiments, systems comprise reagents for nucleic acid amplification of a target nucleic acid in a sample. Nucleic acid amplification of the target nucleic acid may improve at least one of sensitivity, specificity, or accuracy of the assay in detecting the target nucleic acid. In some embodiments, nucleic acid amplification is isothermal nucleic acid amplification, providing for the use of the system or system in remote regions or low resource settings without specialized equipment for amplification. In some embodiments, amplification of the target nucleic acid increases the concentration of the target nucleic acid in the sample relative to the concentration of nucleic acids that do not correspond to the target nucleic acid.

The reagents for nucleic acid amplification may comprise a recombinase, a primer, an oligonucleotide primer, deoxynucleoside triphosphates (dNTPs), a single-stranded DNA binding (SSB) protein, Rnase inhibitor, water, a polymerase, reverse transcriptase mix, or a combination thereof that is suitable for an amplification reaction. Non-limiting examples of amplification reactions are transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA). Such amplification reactions may also be used in combination with reverse transcription (RT) of an RNA of interest, such as reverse transcription loop-mediated isothermal amplification (RT-LAMP). Accordingly, also provided herein are reagents for both the reverse transcription and amplification of nucleic acids.

Any reagents suitable with the described amplification reactions are useful as amplification reagents for the systems, compositions, methods, kits, devices, and solutions provided herein, including primers (e.g., LAMP primers), a polymerase (e.g., a DNA polymerase), water (e.g., nuclease free water), dNTPs, reverse transcriptase mix (e.g., master mix) and the like.

In some embodiments, systems, compositions, methods, kits, devices, and solutions comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more of each amplification described herein. In some embodiments, systems, compositions, methods, kits, devices, and solutions comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more of each amplification reagent as described herein. In some embodiments, systems, compositions, methods, kits, devices, and solutions comprise 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM, or more of each amplification reagent as described herein. In some embodiments, systems, compositions, methods, kits, devices, and solutions comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more of each amplification reagent as described herein.

In some embodiments, systems comprise a PCR tube, a PCR well or a PCR plate. The wells of the PCR plate may be pre-aliquoted with the reagent for amplifying a nucleic acid, as well as a guide nucleic acid, an effector protein, a multimeric complex, or any combination thereof. The wells of the PCR plate may be pre-aliquoted with a guide nucleic acid targeting a target sequence, an effector protein capable of being activated when complexed with the guide nucleic acid and the target sequence, and at least one population of a single stranded reporter nucleic acid comprising a detection moiety. A user may thus add the biological sample of interest to a well of the pre-aliquoted PCR plate and measure for the detectable signal with a fluorescent light reader or a visible light reader.

In some embodiments, systems comprise a PCR plate; a guide nucleic acid targeting a target sequence; an effector protein capable of being activated when complexed with the guide nucleic acid and the target sequence; and a single stranded reporter nucleic acid comprising a detection moiety, wherein the reporter nucleic acid is capable of being cleaved by the activated nuclease, thereby generating a detectable signal.

In some embodiments, systems comprise a support medium; a guide nucleic acid targeting a target sequence; and an effector protein capable of being activated when complexed with the guide nucleic acid and the target sequence. In some embodiments, nucleic acid amplification is performed in a nucleic acid amplification region on the support medium. Alternatively, or in combination, the nucleic acid amplification is performed in a reagent chamber, and the resulting sample is applied to the support medium.

In some embodiments, a system for modifying a target nucleic acid comprises a PCR plate; a guide nucleic acid targeting a target sequence; and an effector protein capable of being activated when complexed with the guide nucleic acid and the target sequence. The wells of the PCR plate may be pre-aliquoted with the guide nucleic acid targeting a target sequence, and an effector protein capable of being activated when complexed with the guide nucleic acid and the target sequence. A user may thus add the biological sample of interest to a well of the pre-aliquoted PCR plate.

Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. Sometimes, the nucleic acid amplification is performed for 1 to 60, 5 to 55, 10 to 50, 15 to 45, 20 to 40, or 25 to 35 minutes. Sometimes, the nucleic acid amplification reaction is performed at a temperature of around 20-45° C. In some embodiments, the nucleic acid amplification reaction is performed at a temperature no greater than 20° C., 25° C., 30° C., 35° C., 37° C., 40° C., 45° C., 50° C., 55° C., 60° C. or any value 20° C. to 60° C. In some embodiments, the nucleic acid amplification reaction is performed at a temperature of at least 20° C., 25° C., 30° C., 35° C., 37° C., 40° C., 45° C., 50° C., 55° C., 60° C. or any value 20° C. to 60° C. In some embodiments, the nucleic acid amplification reaction is performed at a temperature of 20° C. to 45° C., 25° C. to 40° C., 30° C. to 40° C., 35° C. to 40° C., 40° C. to 45° C., 45° C. to 50° C., 50° C. to 55° C., 55° C. to 60° C.

Often, systems comprise primers for amplifying a target nucleic acid to produce an amplification product comprising the target nucleic acid and a PAM. For embodiment, at least one of the primers may comprise the PAM that is incorporated into the amplification product during amplification. The compositions for amplification of target nucleic acids and methods of use thereof, as described herein, are compatible with any of the methods disclosed herein including methods of assaying for at least one base difference (e.g., assaying for a SNP or a base mutation) in a target nucleic acid sequence, methods of assaying for a target nucleic acid that lacks a PAM by amplifying the target nucleic acid sequence to introduce a PAM, and compositions used in introducing a PAM via amplification into the target nucleic acid sequence.

### Certain System Conditions

In some embodiments, systems and methods are employed under certain conditions that enhance an activity of the effector protein relative to alternative conditions, as measured by a detectable signal released from cleavage of a reporter in the presence of the target nucleic acid. The detectable signal may be generated at about the rate of trans cleavage of a reporter nucleic acid. In some embodiments, the reporter nucleic acid is a homopolymeric reporter nucleic acid comprising 5 to 20 consecutive adenines (SEQ ID NO: 159), 5 to 20 consecutive thymines (SEQ ID NO: 160), 5 to 20 consecutive cytosines (SEQ ID NO: 161), 5 to 20 consecutive guanines (SEQ ID NO: 162), or any other suitable reporter set forth in TABLE 12.

In some embodiments, effector proteins disclosed herein recognize, bind, or are activated by, different target nucleic acids having different sequences, but are active toward the same reporter nucleic acid, allowing for facile multiplexing in a single assay.

In some embodiments, systems are employed under certain conditions that enhance trans cleavage activity of an effector protein. In some embodiments, under certain conditions, trans-colatteral cleavage occurs at a rate of at least 0.005 mmol/min, at least 0.01 mmol/min, at least 0.05 mmol/min, at least 0.1 mmol/min, at least 0.2 mmol/min, at least 0.5 mmol/min, or at least 1 mmol/min. In some embodiments, systems and methods are employed under certain conditions that enhance cis cleavage activity of the effector protein.

Certain conditions that may enhance the activity of an effector protein include a certain salt presence or salt concentration of the solution in which the activity occurs. For example, cis cleavage activity of an effector protein may be inhibited or halted by a high salt concentration. The salt may be a magnesium salt, a zinc salt, a potassium salt, a calcium salt, a lithium salt, an ammonium salt, or a sodium salt. In some embodiments, the salt is magnesium acetate. In some embodiments, the salt is magnesium chloride. In some embodiments, the salt is potassium acetate. In some embodiments, the salt is potassium nitrate. In some embodiments, the salt is zinc chloride. In embodiments, the salt is sodium chloride. In some embodiments, the salt is potassium chloride. In some embodiments, the salt is lithium acetate. In some embodiments, the salt is ammonium sulfate. In some embodiments, the salt concentration is less than 150 mM, less than 125 mM, less than 100 mM, less than 75 mM, less than 50 mM, or less than 25 mM. In some embodiments, the salt concentration is more than 1 mM, but less than 150 mM, less than 125 mM, less than 100 mM, less than 75 mM, less than 50 mM, or less than 25 mM. In some embodiments, the salt concentration is more than 10 mM, but less than 150 mM, less than 125 mM, less than 100 mM, less than 75 mM, less than 50 mM, or less than 25 mM. In some embodiments, the salt is potassium acetate or sodium chloride and the concentration of salt in the solution is about 200 mM. In some embodiments, the salt is potassium acetate, sodium chloride, lithium acetate, or ammonium sulfate and the concentration of salt in the solution is about 100 mM to about 200 mM.

Certain conditions that may enhance the activity of an effector protein include the pH of a solution in which the activity. For example, increasing pH may enhance trans cleavage activity. For example, the rate of trans cleavage activity may increase with increase in pH up to pH 9. In some embodiments, the pH is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH is less than 7. In some embodiments, the pH is greater than 7.

Certain conditions that may enhance the activity of an effector protein includes the temperature at which the activity is performed. In some embodiments, the temperature is about 25° C. to about 50° C. In some embodiments, the temperature is about 20° C. to about 40° C., about 30° C. to about 50° C., or about 40° C. to about 60° C. In some embodiments, the temperature is about 25° C., about 30° C., about 35° C., about 40° C., about 45° C., 50° C., about 55° C., or about 60° C.

### Kits for Detection

In some embodiments, system components are assembled in a kit. Accordingly, disclosed herein are kits for modifying, and/or detecting target nucleic acid. In general, kit components comprise structural components as well as sample components, including compositions and systems described herein. Often, kits comprise one or more containers compatible for containing the samples, compositions, and systems described herein. In some embodiments, components of the samples, compositions, and systems are contained in the same container or in separate containers. In some embodiments, a container is a syringe, test wells, bottles, chambers, channels, vials, or test tubes. In some embodiments, kits comprise components comprising one or more of: compositions described herein; systems described herein; components thereof; other components as described herein; or combinations thereof. In some embodiments, kits are compatible with any methods disclosed herein, including methods used for detection, treatment, and/or diagnosis of a disease or disorder.

### Kit Components

In some embodiments, kits described herein include a package, carrier, or container that is compartmentalized to receive one or more containers such as syringes, vials, tubes, and the like, each of the container(s) comprising one of the separate elements to be used in a method described herein. Suitable containers include, for example, syringes, test wells, chambers, channels, bottles, vials, and test tubes. In one embodiment, the containers are formed from a variety of materials such as glass, plastic, or polymers. The system or systems described herein contain packaging materials. Examples of packaging materials include, but are not limited to, pouches, blister packs, bottles, tubes, bags, containers, bottles, and any packaging material suitable for intended mode of use.

A kit described herein may include labels listing contents and/or instructions for use, or package inserts with instructions for use. A set of instructions will also typically be included. In one embodiment, a label is on or associated with the container. In some embodiments, a label is on a container when letters, numbers or other characters forming the label are attached, molded, or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein. After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

In some embodiments, kits described herein comprise a solid support. An RNP or effector protein may be attached to a solid support. The solid support may, for example, be an electrode or a bead. The bead may be a magnetic bead. Upon cleavage, the RNP is liberated from the solid support and interacts with other mixtures. For example, upon cleavage of the nucleic acid of the RNP, the effector protein of the RNP flows through a chamber into a mixture comprising a substrate. When the effector protein meets the substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

### Methods of Detection

Provided herein are methods of detecting target nucleic acids. Methods may comprise detecting target nucleic acids with compositions or systems described herein. Methods may comprise detecting a target nucleic acid in a sample, e.g., a cell lysate, a biological fluid, or environmental sample. Methods may comprise detecting a target nucleic acid in a cell. In some embodiments, methods of detecting a target nucleic acid in a sample or cell comprises contacting the sample or cell with an effector protein or a multimeric complex thereof, a guide nucleic acid, wherein at least a portion of the guide nucleic acid is complementary to at least a portion of the target nucleic acid, and a reporter nucleic acid that is cleaved in the presence of the effector protein, the guide nucleic acid, and the target nucleic acid, and detecting a signal produced by cleavage of the reporter nucleic acid, thereby detecting the target nucleic acid in the sample. In some embodiments, methods result in trans cleavage of the reporter nucleic acid. In some embodiments, methods result in cis cleavage of the reporter nucleic acid. In some embodiments, methods of detecting a target nucleic acid include a reporter nucleic acid comprising a detectable moiety that produces a detectable signal in the presence of the target nucleic acid, the effector protein, and the guide nucleic acid.

In some embodiments, the methods of detecting a target nucleic acid comprising: a) contacting the target nucleic acid with a composition comprising an effector protein as described herein, a guide nucleic acid as described herein, and a reporter nucleic acid that is cleaved in the presence of the effector protein, the guide nucleic acid, and the target nucleic acid; and b) detecting a signal produced by cleavage of the reporter nucleic acid, thereby detecting the target nucleic acid in the sample. In some embodiments, the methods result in trans cleavage of the reporter nucleic acid. In some embodiments, the methods result in cis cleavage of the reporter nucleic acid. In some embodiments, the reporter nucleic acid is a single stranded nucleic acid. In some embodiments, the reporter comprises a detection moiety. In some embodiments, the reporter nucleic acid is capable of being cleaved by the effector protein. In some embodiments, a cleaved reporter nucleic acid generates a detectable product or a first detectable signal. In some embodiments, the first detectable signal is a change in color. In some embodiments, the change is color is measured indicating presence of the target nucleic acid. In some embodiments, the first detectable signal is measured on a support medium.

In some embodiments, methods of detecting comprise contacting a target nucleic acid, a cell comprising the target nucleic acid, or a sample comprising a target nucleic acid with an effector protein that comprises an amino acid sequence that is at least is at least 70%, at least 75%, at least 80%, at least 85%, at least 90%, at least 92%, at least 95%, at least 97%, at least 98%, at least 99%, or 100% identical to any one of the sequences set forth in TABLE 1. In some embodiments, the amino acid sequence of the effector protein is at least 70%, at least 75%, at least 80%, at least 85%, at least 90%, at least 92%, at least 95%, at least 97%, at least 98%, at least 99%, or 100% identical to any one of the sequences set forth in TABLE 1. In some embodiments, the effector protein comprising an amino acid sequence that is at least 90% identical to a sequence selected from any one of the sequences set forth in TABLE 1.

Methods may comprise contacting the sample to a complex comprising a guide nucleic acid comprising a segment that is reverse complementary to a segment of the target nucleic acid and a effector protein that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample.

Methods may comprise contacting the sample comprising the target nucleic acid with a guide nucleic acid targeting a target nucleic acid segment, an effector protein capable of being activated when complexed with the guide nucleic acid and the target nucleic acid segment, a single stranded nucleic acid of a reporter comprising a detection moiety, wherein the nucleic acid of a reporter is capable of being cleaved by the activated effector protein, thereby generating a first detectable signal, cleaving the single stranded nucleic acid of a reporter using the effector protein that cleaves as measured by a change in color, and measuring the first detectable signal on the support medium.

Methods may comprise contacting the sample or cell with an effector protein or a multimeric complex thereof and a guide nucleic acid at a temperature of at least about 25° C., at least about 30° C., at least about 35° C., at least 37° C., at least about 40° C., at least about 50° C., at least about 65° C., at least about 70° C., at least about 75° C. In some embodiments, the temperature is not greater than 80° C. In some embodiments, the temperature is about 25° C., about 30° C., about 35° C., about 37° C., about 40° C., about 45° C., about 50° C., about 55° C., about 60° C., about 65° C., about 70° C., about 75° C., about 80° C., about 85° C. or about 90° C. In some embodiments, the temperature is about 25° C. to about 45° C., about 35° C. to about 55° C., about 37° C. to about 60° C. or about 55° C. to about 65° C. In some embodiments, the temperature is about 37° C. to about 45° C., about 37° C. to about 50° C., about 37° C. to about 55° C., about 37° C. to about 60° C., or about 37° C. to about 65° C.

Methods may comprise contacting the sample or cell with an effector protein or a multimeric complex thereof and a guide nucleic acid in the presence of salts. In some embodiments, the method may comprise a solution, wherein the solution comprises one or more salt. Accordingly, in some embodiments, the salt may be one or more salt selected from a magnesium salt, a zinc salt, a potassium salt, a calcium salt, and a sodium salt. In some embodiments, the salt is a combination of two or more salts. For example, in some embodiments, the salt is a combination of two or more salts selected from a magnesium salt, a zinc salt, a potassium salt, a calcium salt and a sodium salt. In some embodiments, the salt is magnesium acetate. In some embodiments, the salt is magnesium chloride. In some embodiments, the salt is potassium acetate. In some embodiments, the salt is potassium nitrate. In some embodiments, the salt is zinc chloride. In embodiments, the salt is sodium chloride. In some embodiments, the salt is potassium chloride. In some embodiments, the concentration of the one or more salt in the solution is about 0.001 mM to about 500 mM. In some embodiments, the concentration of the salt is about 0.001 mM to about 400 mM. In some embodiments, the concentration of the salt is about 0.001 mM to about 300 mM. In some embodiments, the concentration of the salt is about 0.001 mM to about 200 mM. In some embodiments, the concentration of the salt is about 0.001 mM to about 100 mM. In some embodiments, the concentration of the salt is about 0.001 mM to about 10 mM. In some embodiments, the concentration of the salt is about 0.01 mM to about 500 mM. In some embodiments, the concentration of the salt is about 0.01 mM to about 400 mM. In some embodiments, the concentration of the salt is about 0.01 mM to about 300 mM. In some embodiments, the concentration of the salt is about 0.01 mM to about 200 mM. In some embodiments, the concentration of the salt is about 0.01 mM to about 100 mM. In some embodiments, the concentration of the salt is about 0.01 mM to about 10 mM. In some embodiments, the concentration of the salt is about 0.1 mM to about 500 mM. In some embodiments, the concentration of the salt is about 0.1 mM to about 400 mM. In some embodiments, the concentration of the salt is about 0.1 mM to about 300 mM. In some embodiments, the concentration of the salt is about 0.1 mM to about 200 mM. In some embodiments, the concentration of the salt is about 0.1 mM to about 100 mM. In some embodiments, the concentration of the salt is about 0.1 mM to about 10 mM. In some embodiments, the concentration of the salt is about 1 mM to about 500 mM. In some embodiments, the concentration of the salt is about 1 mM to about 400 mM. In some embodiments, the concentration of the salt is about 1 mM to about 300 mM. In some embodiments, the concentration of the salt is about 1 mM to about 200 mM. In some embodiments, the concentration of the salt is about 1 mM to about 100 mM. In some embodiments, the concentration of the salt is about 1 mM to about 10 mM. In some embodiments, the concentration of the salt is about 10 mM to about 500 mM. In some embodiments, the concentration of the salt is about 10 mM to about 400 mM. In some embodiments, the concentration of the salt is about 10 mM to about 300 mM. In some embodiments, the concentration of the salt is about 10 mM to about 200 mM. In some embodiments, the concentration of the salt is about 10 mM to about 100 mM. In some embodiments, the concentration of the salt is about 100 mM to about 500 mM. In some embodiments, the concentration of the salt is about 100 mM to about 400 mM. In some embodiments, the concentration of the salt is about 100 mM to about 300 mM. In some embodiments, the concentration of the salt is about 100 mM to about 200 mM. In some embodiments, the salt is potassium acetate and the concentration of salt in the solution is about 100 mM. In some embodiments, the salt is potassium acetate or sodium chloride and the concentration of salt in the solution is about 200 mM. In some embodiments, the salt is potassium acetate or sodium chloride and the salt of potassium in the solution is about 100 mM to about 200 mM.

In some embodiments, methods of detecting a target nucleic acid by a cleavage assay. In some embodiments, the target nucleic acid is a single-stranded target nucleic acid. In some embodiments, the cleavage assay comprises: a) contacting the target nucleic acid with a composition comprising an effector protein as described; and b) cleaving the target nucleic acid. In some embodiments, the cleavage assay comprises an assay designed to visualize, quantitate or identify cleavage of a nucleic acid. In some embodiments, the method is an in vitro trans-cleavage assay. In some embodiments, a cleavage activity is a trans-cleavage activity. In some embodiments, the method is an in vitro cis-cleavage assay. In some embodiments, a cleavage activity is a cis-cleavage activity. In some embodiments, the cleavage assay follows a procedure comprising: (i) providing a composition comprising an equimolar amounts of an effector protein as described herein, and a guide nucleic acid described herein, under conditions to form an RNP complex; (ii) adding a plasmid comprising a target nucleic acid, wherein the target nucleic acid is a linear dsDNA, wherein the target nucleic acid comprises a target sequence and a PAM (iii) incubating the mixture under conditions to enable cleavage of the plasmid; (iv) quenching the reaction with EDTA and a protease; and (v) analyzing the reaction products (e.g., viewing the cleaved and uncleaved linear dsDNA with gel electrophoresis).

In some embodiments, there is a threshold of detection for methods of detecting target nucleic acids. In some embodiments, methods are not capable of detecting target nucleic acids that are present in a sample or solution at a concentration less than or equal to 10 nM. For example, when a threshold of detection is 10 nM, then a signal can be detected when a target nucleic acid is present in the sample at a concentration of 10 nM or more. In some embodiments, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some embodiments, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 pM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, 1 pM to 200 pM, 1 pM to 100 pM, or 1 pM to 10 pM. In some embodiments, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some embodiments, the threshold of detection is in a range of from 2 aM to 100 pM, from 20 aM to 50 pM, from 50 aM to 20 pM, from 200 aM to 5 pM, or from 500 aM to 2 pM.

In some embodiments, the target nucleic acid is present in a cleavage reaction at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, the target nucleic acid is present in the cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, the target nucleic acid is present in the cleavage reaction at a concentration of from 20 nM to 50 μM, from 50 nM to 20 μM, or from 200 nM to 5 μM.

In some embodiments, methods detect a target nucleic acid in less than 60 minutes. In some embodiments, methods detect a target nucleic acid in less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 5 minutes, less than about 4 minutes, less than about 3 minutes, less than about 2 minutes, or less than about 1 minute.

Methods may comprise detecting a detectable signal within 5 minutes of contacting the sample and/or the target nucleic acid with the guide nucleic acid and/or the effector protein. In some embodiments, detecting occurs within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the target nucleic acid. In some embodiments, detecting occurs within 1 to 120, 5 to 100, 10 to 90, 15 to 80, 20 to 60, or 30 to 45 minutes of contacting the target nucleic acid.

In some embodiments, methods of detecting as disclosed herein are compatible with methods for diagnosis of a disease or disorder.

### Amplification of a Target Nucleic Acid

Methods may comprise amplifying a target nucleic acid for detection using any of the compositions or systems described herein. Amplifying may comprise changing the temperature of the amplification reaction, also known as thermal amplification (e.g., PCR). Amplifying may be performed at essentially one temperature, also known as isothermal amplification. Amplifying may improve at least one of sensitivity, specificity, or accuracy of the detection of the target nucleic acid.

Amplifying may comprise subjecting a target nucleic acid to an amplification reaction selected from transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

In some embodiments, amplification of the target nucleic acid comprises modifying the sequence of the target nucleic acid. For example, amplification may be used to insert a PAM sequence into a target nucleic acid that lacks a PAM sequence. In some embodiments, amplification may be used to increase the homogeneity of a target nucleic acid in a sample. For example, amplification may be used to remove a nucleic acid variation that is not of interest in the target nucleic acid sequence.

Amplifying may take 1, 2, 3, 4, 5, 6, 7, 8, 9, 10, 11, 12, 13, 14, 15, 16, 17, 18, 19, 20, 25, 30, 40, 50, or 60 minutes. Amplifying may be performed at a temperature of around 20-45° C. Amplifying may be performed at a temperature of less than about 20° C., less than about 25° C., less than about 30° C., 35° C., less than about 37° C., less than about 40° C., or less than about 45° C. The nucleic acid amplification reaction may be performed at a temperature of at least about 20° C., at least about 25° C., at least about 30° C., at least about 35° C., at least about 37° C., at least about 40° C., or at least about 45° C.

### Amplification and Detection of a Target Nucleic Acid

Described herein are various methods of sample amplification and detection in a single reaction volume. Any of the devices described herein may be configured to perform amplification and detection in a same well, chamber, channel, or volume in the device. In some embodiments, methods include simultaneous amplification and detection in the same volume. In some embodiments, methods include sequential amplification and detection in the same volume. In some embodiments, amplification and detection may occur in a single pot or a one-pot reaction, where reverse transcription, amplification, in vitro transcription, or any combination thereof, and detection are carried out in a single volume. Any suitable method of reverse transcription, amplification, in vitro transcription, and detection can be used in such a reaction, such as methods of reverse transcription, amplification, in vitro transcription, and detection described herein. In some embodiments, amplification and detection may occur in a HotPot reaction.

In some embodiments, a DETECTR reaction may be used to detect the presence of a specific target gene in the same. The DETECTR reaction may produce a detectable signal, as described elsewhere herein, in the presence of a target nucleic acid sequence comprising a target gene. The DETECTR reaction may not produce a signal in the absence of the target nucleic acid or in the presence of a nucleic acid sequence that does not comprise the specific SNP allele or comprises a different SNP allele. In some embodiments, a DETECTR reaction may comprise a guide RNA reverse complementary to a portion of a target nucleic acid sequence comprising a specific SNP allele. The guide RNA and the target nucleic acid comprising the specific SNP allele may bind to and activate a effector protein, thereby producing a detectable signal as described elsewhere herein. The guide RNA and a nucleic acid sequence that does not comprise the specific SNP allele may not bind to or activate the effector protein and may not produce a detectable signal. In some embodiments, a target nucleic acid sequence that may or may not comprise a specific SNP allele may be amplified using, for example, a LAMP amplification reaction, an RPA amplification reaction, an SDA amplification reaction, a NEAR amplification reaction, or any other amplification method. In some embodiments, the amplification reaction may be combined with a reverse transcription reaction, a DETECTR reaction, or both. For example, the amplification reaction may be an RT-NEAR reaction, a NEAR DETECTR reaction, or an RT-NEAR DETECTR reaction. In some embodiments, the target nucleic acid sequence can comprise a SNP. In some embodiments, the target nucleic acid sequence can comprise a sequence indicative of a human disease.

A DETECTR reaction, as described elsewhere herein, may produce a detectable signal specifically in the presence of a target nucleic acid sequence comprising a target gene. In addition to the DETECTR reaction, the target nucleic acid having the target gene may be concurrently, sequentially, concurrently together in a sample, or sequentially together in a sample be carried out alongside NEAR or RT-NEAR. For example, the reactions can comprise NEAR and DETECTR reactions, or RT-NEAR and DETECTR reactions. Performing a DETECTR reaction in combination with a NEAR reaction may result in an increased detectable signal as compared to the DETECTR reaction in the absence of the NEAR reaction. In some embodiments, the target nucleic acid sequence can comprise a SNP. In some embodiments, the target nucleic acid sequence can comprise a sequence indicative of a human disease.

In some embodiments, the detectable signal produced in the DETECTR reaction may be higher in the presence of a target nucleic acid comprising target nucleic acid than in the presence of a nucleic acid that does not comprise the target nucleic acid. In some embodiments, the DETECTR reaction may produce a detectable signal that is at least 1-fold, at least 2-fold, at least 3-fold, at least 4-fold, at least 5-fold, at least 10-fold, at least 20-fold, at least 30-fold, at least 40-fold, at least 50-fold, at least 100-fold, at least 200-fold, at least 300-fold, at last 400-fold, at least 500-fold, at least 1000-fold, at least 2000-fold, at least 3000-fold, at least 4000-fold, at least 5000-fold, at least 6000-fold, at least 7000-fold, at least 8000-fold, at least 9000-fold, at least 10000-fold, at least 50000-fold, at least 100000-fold, at least 500000-fold, or at least 1000000-fold greater in the presence of a target nucleic acid comprising a target nucleic acid than in the presence of a nucleic acid that does not comprise the target nucleic acid. In some embodiments, the DETECTR reaction may produce a detectable signal that is from 1-fold to 2-fold, from 2-fold to 3-fold, from 3-fold to 4-fold, from 4-fold to 5-fold, from 5-fold to 10-fold, from 10-fold to 20-fold, from 20-fold to 30-fold, from 30-fold to 40-fold, from 40-fold to 50-fold, from 50-fold to 100-fold, from 100-fold to 500-fold, from 500-fold to 1000-fold, from 1000-fold to 10,000-fold, from 10,000-fold to 100,000-fold, or from 100,000-fold to 1,000,000-fold greater in the presence of a target nucleic acid comprising a specific SNP allele than in the presence of a nucleic acid that does not comprise the specific SNP allele. In some embodiments, the target nucleic acid sequence can comprise a SNP. In some embodiments, the target nucleic acid sequence can comprise a sequence indicative of a human disease.

A DETECTR reaction may be used to detect the presence of a target nucleic acid associated with a disease or a condition in a nucleic acid sample. The DETECTR reaction may reach signal saturation within about 30 seconds, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 75 minutes, about 80 minutes, or about 85 minutes and be used to detect the presence of a target gene associated with an increased likelihood of developing a disease or a condition in a nucleic acid sample. The DETECTR reaction may be used to detect the presence of a target gene associated with a phenotype in a nucleic acid sample. For example, a DETECTR reaction may be used to detect target gene associated with a disease such as phenylketonuria (PKU), cystic fibrosis, sickle-cell anemia, albinism, Huntington's disease, myotonic dystrophy type 1, hypercholesterolemia, neurofibromatosis, polycystic kidney disease, hemophilia, muscular dystrophy, hypophosphatemic rickets, Rett's syndrome, or spermatogenic failure. A DETECTR reaction may be used to detect a SNP allele associated with an increased risk of cancer, for example bladder cancer, brain cancer, breast cancer, cervical cancer, colon cancer, colorectal cancer, gallbladder cancer, stomach cancer, leukemia, liver cancer, lung cancer, oral cancer, esophageal cancer, ovarian cancer, pancreatic cancer, prostate cancer, skin cancer, testicular cancer, thyroid cancer, neuroblastoma, or lymphoma. A DETECTR reaction may be used to detect a SNP allele associated with an increased risk of a disease, for example Alzheimer's disease, Parkinson's disease, amyloidosis, heterochromatosis, celiac disease, macular degeneration, or hypercholesterolemia. A DETECTR reaction may be used to detect a SNP allele associated with a phenotype, for example, eye color, hair color, height, skin color, race, alcohol flush reaction, caffeine consumption, deep sleep, genetic weight, lactose intolerance, muscle composition, saturated fat and weight, or sleep movement. A DETECTR reaction may also be used to detect the presence of a pathological organism. In some embodiments, the pathological organism is a prokaryote, eukaryote, or a protozoa. In some embodiments, the pathological organism is a virus, an opportunistic pathogen, a parasite, a bacterium, or any combination thereof. In some embodiments, the pathological organism is SARS-CoV-2 or Streptococcus pyogenes.

### Devices

Disclosed herein are devices for modifying and/or detecting target nucleic acid. In some embodiments, devices comprise components comprising one or more of: compositions described herein; systems described herein; other components or appurtenances as described herein; or combinations thereof.

### Device Components

In general, device components comprise a structural component as well as sample components, including compositions, solutions, and systems described herein. Often, a sample component comprises or consists essentially of compositions, systems, or components thereof as described herein. Additional device components may comprise one or more hydrogels or surfaces with immobilized reporters. In some embodiments, a device's sample component may be contained in at least one structural device component, such as a sample interface, which may be in fluid communication with a chamber. In some embodiments, the sample interface is fluidically connected to a chamber. In some embodiments, by being in fluid communication with a chamber, a device's sample component may flow from the sample interface into a chamber. In some embodiments, a device's sample component may flow from the sample interface into a chamber by way of the fluid connection. By way of non-limiting example, a device's sample component may be simultaneously contained in a sample interface and a chamber.

In some embodiments, the devices described herein may comprise a plurality of hydrogels each comprising reporter molecules (e.g., in order to facilitate multiplexing and/or improve signal). In some embodiments, a first hydrogel may comprise a shape different from a shape of a second hydrogel. In some embodiments, the first hydrogel may comprise a plurality of first reporter molecules different from a plurality of second reporter molecules of the second hydrogel. In some embodiments, the reporters are the same in the first and second hydrogels. In some embodiments, the first hydrogel may comprise a circular shape, a square shape, a star shape, or any other shape distinguishable from a shape of the second hydrogel. In some embodiments, the plurality of first reporter molecules may each comprise a sequence cleavable by an effector protein-guide nucleic acid complex comprising a first effector protein and a first guide nucleic acid. In some embodiments, the plurality of second reporter molecules may each comprise a sequence not cleavable by the first effector protein-guide nucleic acid complex.

Any of the devices described herein may comprise a plurality of hydrogels each comprising reporter molecules. For example, a first hydrogel may comprise a plurality of first reporter molecules different from a plurality of second reporter molecules of a second hydrogel. In some embodiments, the plurality of first reporter molecules may each comprise a first fluorescent moiety, wherein the first fluorescent moiety is different than second fluorescent moieties of in each of the plurality of second reporter molecules. In some embodiments, the plurality of first reporter molecules may each comprise a sequence cleavable by a first effector protein complex comprising a first effector protein and a first guide nucleic acid. In some embodiments, the plurality of second reporter molecules may each comprise a sequence cleavable by a second effector protein complex comprising a second effector protein and a second guide nucleic acid.

Any of the devices described herein may comprise at least about 2 hydrogels, at least about 3 hydrogels, at least about 4 hydrogels, at least about 5 hydrogels, at least about 6 hydrogels, at least about 7 hydrogels, at least about 8 hydrogels, at least about 9 hydrogels, at least about 10 hydrogels, at least about 20 hydrogels, at least about 30 hydrogels, at least about 40 hydrogels, at least about 50 hydrogels, at least about 60 hydrogels, at least about 70 hydrogels, at least about 80 hydrogels, at least about 90 hydrogels, at least about 100 hydrogels, at least about 200 hydrogels, at least about 300 hydrogels, at least about 400 hydrogels, at least about 500 hydrogels, at least about 600 hydrogels, at least about 700 hydrogels, at least about 800 hydrogels, at least about 900 hydrogels, at least about 1000 hydrogels,

Any of the devices described herein may comprise one or more compartments, chambers, channels, or locations comprising the one or more hydrogels or surfaces. In some embodiments, two or more of the compartments or chambers may be in fluid communication, optical communication, thermal communication, or any combination thereof with one another. In some embodiments, two or more compartments or chambers may be arranged in a sequence. In some embodiments, two or more compartments or chambers may be arranged in parallel. In some embodiments, two or more compartments or chambers may be arranged in sequence, parallel, or both. In some embodiments, one or more compartments or chambers may comprise a well. In some embodiments, one or more compartments or chambers may comprise a flow strip. In some embodiments, one or more compartments or chambers may comprise a heating element.

Any of the devices described herein may comprise a sample interface, which may be in fluid communication with a valve and/or a chamber, such as comprising configuration to be fluidically connected to a valve and/or chamber. In some embodiments, a device's sample component may flow from the sample interface, through a valve, and into a chamber. In some embodiments, a valve disposed between the sample interface and the chamber comprise configuration to selectively resist flow or permit flow. In some embodiments, a chamber comprise configuration to comprise compositions, systems, one or more reagents for amplification (i.e., amplification reagents), one or more reagents for detection (i.e., detection reagents), one or more cell lysis reagents, one or more nucleic acid purification reagents, or combinations thereof. In some embodiments, a chamber and/or a valve comprise configuration to be thermally connected to a heating element.

Any of the devices described herein may comprise a plurality of chambers and/or a plurality of valves configured to be fluidically connected. In some embodiments, a plurality of valves comprise configuration to restrict flow in a first direction through channels and/or sample interface. In some embodiments, a plurality of valves comprise configuration to restrict flow in a second direction through channels and/or a reaction chamber. In some embodiments, a plurality of valves comprise configuration to comprise a valve inlet channel and/or a valve outlet channel. In some embodiments, a plurality of valves comprise configuration to simultaneously or independently be in an open state or a closed state. In some embodiments, a plurality of valves comprise configuration to physically, fluidically, or thermally isolate a first portion of a sample from a second portion of a sample when a first valve and a second valve are in a closed state.

In some embodiments, a plurality of chambers may comprise a first chamber and a second chamber, wherein the second chamber is disposed between the sample interface and the first chamber. In some embodiments, a second chamber is disposed fluidically downstream of the sample interface and the first chamber. In some embodiments, a second chamber is disposed upstream of the sample interface and the first chamber. In some embodiments, a first chamber is disposed to be fluidically connected to a detection region.

In some embodiments, the device may comprise one or more lateral flow assay strips in a detection region disposed downstream of a reaction chamber. In some embodiments, the device may comprise one or more lateral flow assay strips in a detection region which may be brought into fluid communication with the reaction chamber. Each lateral flow assay strip contains one or more detection regions or spots, where each detection region or spot contains a different type of capture antibody. In some embodiments, each lateral flow assay strip contains a different type of capture antibody. In some embodiments, each capture antibody type specifically binds to a particular label type of a reporter. In some embodiments, the reaction chamber may comprise one or more guide nucleic acids (e.g., sgRNAs), and/or effector proteins described herein.

In general, the buffers described herein are compatible for use in the devices described herein (e.g., pneumatic valve devices, sliding valve devices, rotating valve devices, lateral flow devices, and microfluidic devices). In some embodiments, the device may be a microfluidic device. In some embodiments, the device may be a handheld device. In some embodiments, the device may be point-of-need device. In some embodiments, the device may comprise any one of the device configurations described herein. In some embodiments, the device may comprise one or more parts of any one of the device configurations described herein.

Generally, a sample comprises one or more target nucleic acids and a chamber (e.g., a reaction chamber) comprises one or more of: effector proteins, guide nucleic acids, and reporters comprising a nucleic acid and a detection moiety. In some embodiments, a sample may flow from a sample interface into a chamber by way of the fluid connection wherein the sample may interact with the components of the compositions, systems, and solutions contained therein. Upon interacting, an effector protein and a guide nucleic acid are capable of forming an effector protein-guide nucleic acid complex (e.g., an RNP). An effector protein may become activated after binding of a guide nucleic acid, that is complexed with the effector protein, with a target nucleic acid, and the activated effector protein may cleave the target nucleic acid, which can result in a trans cleavage activity. Trans cleavage activity can be non-specific cleavage of nearby single-stranded nucleic acids by the activated effector protein, such as trans cleavage of the nucleic acid (e.g., a detector nucleic acid) with a detection moiety of the reporter. Once the target nucleic acid is cleaved by the activated effector protein, the detection moiety can be released or separated from the reporter and can directly or indirectly generate a detectable signal. The reporter and/or the detection moiety can be immobilized on a support medium, such as a surface within the device. Often the detection moiety is at least one of a fluorophore, a dye, a polypeptide, or a nucleic acid. Sometimes the detection moiety binds to a capture molecule on the support medium to be immobilized. The detectable signal can be visualized on the support medium to assess the presence or concentration of one or more target nucleic acids associated with an ailment, such as a disease, cancer, or genetic disorder.

Any of the devices described herein are compatible with any of the compositions, systems, kits, or methods disclosed herein, including methods of detecting and treating a disease or disorder. By way of non-limiting example, the devices described herein may be used in diagnosis of a disease or disorder, for example a disease or disorder set forth in TABLE 9. In some embodiments, the devices described herein may be used in detection of a modified nucleic acid sequence associated with a disease or disorder associated gene. Also, by way of non-limiting example, the devices described herein are compatible with detection of a nucleic acid sequence selected from a viral genome, a prokaryotic genome, or a eukaryotic genome.

### Microfluidic Devices

Disclosed herein are microfluidic devices and uses thereof, e.g., use for detection of target nucleic acids. Devices described herein can be used for an effector protein-based detection (e.g., DETECTR) assay. In some embodiments, the devices are compatible with multiplex lateral flow detection. In some embodiments, the devices are configured to perform one or more of the reactions described herein (e.g., amplification, detection, etc.) in separate chambers. In some embodiments, isolating portions of a liquid sample for detection of different target nucleic acids may facilitate multiplexing (e.g., by air gaps separating the liquid contents of various chambers during a reaction). In some embodiments, the devices described herein can be used in combination with enzyme-based methods for signal amplification of a binding event between one or more effector protein probes and one or more target nucleic acids. In some embodiments, signal detection is performed on the device (e.g., in a reaction chamber, or in a detection chamber connected to the reaction chamber). In some embodiments, the device is configured to allow removal of the contents of a reaction chamber to perform a signal detection step. Methods for signal detection compatible with the devices are also disclosed herein.

In some embodiments, described herein is a microfluidic device comprising: a sample interface configured to receive a sample; and a chamber fluidically connected to the sample interface. In some embodiments, the sample for use in the microfluidic device comprises one or more nucleic acids, for example, one or more target nucleic acids. In some embodiments, the sample for use in the microfluidic device comprises one or more target nucleic acids, for example, one or more target nucleic acids as set forth in TABLE 7 herein. Suitable sample conditions are also described herein and include suitable target copy numbers, solutions, and the like.

In some embodiments, the chamber of the microfluidic device comprises one or more components of the compositions, systems, or solutions described herein. In some embodiments, the chamber of the microfluidic device comprises one or more of: an effector protein, a guide nucleic acid, a reporter, or any combination thereof. In some embodiments, the chamber comprises a reporter comprising a nucleic acid and a detection moiety. Suitable reporters are described herein, for example in TABLE 12.

In some embodiments, microfluidic devices described herein comprise a sample interface configured to receive a sample, wherein the sample comprises one or more target nucleic acids; and a chamber fluidically connected to the sample interface, wherein the chamber comprises an effector protein described herein, an engineered guide nucleic acid described herein, a reporter comprising a nucleic acid and a detection moiety, and reagents (e.g., detection reagents); wherein the sample comprising the target nucleic acids, the effector protein, the engineered guide nucleic acid, and the reporter are able to interact by way of the fluid connection. As described herein, the effector protein and the engineered guide nucleic acid contained in the chamber are capable of forming an activated complex upon hybridization of the engineered guide nucleic acid to a target sequence of a target nucleic acid and wherein the nucleic acid of the reporter is a cleavage substrate of the activated complex. The activated complex is capable cleaving the nucleic acid of the reporter (i.e., the detection event), releasing the detection moiety and thereby allowing it to generate a detectable signal. Accordingly, in some embodiments, a target nucleic acid is detected in the form of a signal (i.e., a detectable signal or detectable product) as a result of the reaction between the sample liquid, or a portion thereof, and the effector protein-based reagents, as described herein.

In some embodiments, the microfluidic device further comprises a valve disposed between the sample interface and the chamber. In some embodiments, the valve is configured to selectively resist flow, or permit flow of the sample components and the chamber components as described herein. Nonlimiting examples of valves include phase-change valves, wax valves, capillary valves, electrostatic valves, check valves, sliding valves, rotary valves, pneumatic valves, vacuum valves, pinch valves, and burst valves.

In some embodiments, the chamber further comprises one or more: amplification reagents, detection reagents, cell lysis reagents, and/or nucleic acid purification reagents. Amplification reagents, detection reagents, cell lysis reagents, and/or nucleic acid purification reagents are described herein, for example, in the Examples. In some embodiments, the chamber further comprises a polymerase, for example a DNA polymerase or an RNA polymerase. Other suitable polymerases are described herein.

In some embodiments, the chamber is a first chamber and the microfluidic device further comprising a second chamber comprising one or more: amplification reagents, detection reagents, cell lysis reagents, and/or nucleic acid purification reagents. In some embodiments, the second chamber or channel is disposed between the sample interface and the first chamber, wherein the second chamber or channel is disposed downstream of the sample interface and the first chamber, wherein the second chamber or channel is disposed upstream of the sample interface and the first chamber.

In some embodiments, the microfluidic device further comprises a detection region fluidically connected to the first chamber. In some embodiments, the detection region comprises an array, one or more lateral flow strips, a detection tray, a detection region comprising a capture antibody, or combinations thereof.

It is understood that description of device configurations or components herein, also describes microfluidic device configuration or components provided herein and vice versa. Accordingly, in some embodiments, the microfluidic device may comprise any one of the device configurations described herein. In still some embodiments, the microfluidic device may comprise one or more parts of any one of the device configurations described herein.

Also disclosed herein is a method of microfluidic devices described herein for target nucleic acid detection. In some embodiments, the method comprises applying a sample to the sample interface. In some embodiments, said applying forms a sample liquid. In some embodiments, the method can comprise sample collection. The method can further comprise sample preparation. In some embodiments, the method may comprise using a physical filter to filter one or more particles from the sample that do not comprise the at least one analyte of interest (e.g., a target nucleic acid). In some embodiments, the method may comprise lysing the sample before detecting the analyte. In some embodiments, the method may comprise performing enzyme (e.g., Proteinase K or savinase) inactivation on the sample. In some embodiments, the method may comprise performing heat inactivation on the sample. In some embodiments, the method may comprise performing nucleic acid purification on the sample. In some embodiments, the method may comprise contacting a plurality of sub-samples with a plurality of effector protein probes comprising different guide RNAs. In some embodiments, the sample is diluted with a buffer or a fluid or concentrated prior to application to the detection system.

In some embodiments, the sample can be provided manually to the device of the present disclosure. For example, a swab sample can be dipped into a solution and the sample/solution can be pipetted into the device. In other embodiments, the sample can be provided via an automated syringe. The automated syringe can be configured to control a flow rate at which the sample is provided to the device. The automated syringe can be configured to control a volume of the sample that is provided to the device over a predetermined period.

In some embodiments, the sample can be provided directly to the device of the present disclosure. For example, a swab sample can be inserted into a sample chamber on the device.

The sample can be prepared before one or more targets are detected within the sample. The sample preparation steps described herein can process a crude sample to generate a pure or purer sample. Sample preparation may comprise one or more physical or chemical processes, including, for example, nucleic acid purification, lysis, binding, washing, and/or eluting. In certain embodiments, sample preparation can comprise the following steps, including sample collection, nucleic acid purification, heat inactivation, enzyme inactivation, and/or base/acid lysis.

In some embodiments, nucleic acid purification can be performed on the sample. Purification can comprise disrupting a biological matrix of a cell to release nucleic acids, denaturing structural proteins associated with the nucleic acids (nucleoproteins), inactivating nucleases that can degrade the isolated product (RNase and/or DNase), and/or removing contaminants (e.g., proteins, carbohydrates, lipids, biological or environmental elements, unwanted nucleic acids, and/or other cellular debris).

In some embodiments, lysis of a collected sample can be performed. Lysis can be performed using a protease (e.g., a Proteinase K or PK enzyme). Exemplary proteases include serine proteases (e.g., Proteinase K, Savinase®, trypsin, Protamex®, etc.), metalloproteinases (e.g., MMP-3, etc.), cysteine proteases (e.g., cathepsin B, papin, etc.), threonine proteases, aspartic proteases (e.g., renin, pepsin, cathepsin D, etc.), glutamic proteases, asparagine peptide lyases, or the like. In some cases, a solution of reagents can be used to lyse the cells in the sample and release the nucleic acids so that they are accessible to the effector protein. Active ingredients of the solution can be chaotropic agents, detergents, salts, and can be of high osmolality, ionic strength, and pH. Chaotropic agents or chaotropes are substances that disrupt the three-dimensional structure in macromolecules such as proteins, DNA, or RNA. One example protocol may comprise a 4 M guanidinium isothiocyanate, 25 mM sodium citrate.2H20, 0.5% (w/v) sodium lauryl sarcosinate, and 0.1 M β-mercaptoethanol), but numerous commercial buffers for different cellular targets can also be used. Alkaline buffers can also be used for cells with hard shells, particularly for environmental samples. Detergents such as sodium dodecyl sulphate (SDS) and cetyl trimethylammonium bromide (CTAB) can also be implemented to chemical lysis buffers. Cell lysis can also be performed by physical, mechanical, thermal or enzymatic means, in addition to chemically-induced cell lysis mentioned previously. In some cases, depending on the type of sample, nanoscale barbs, nanowires, acoustic generators, integrated lasers, integrated heaters, and/or microcapillary probes can be used to perform lysis.

In certain instances, heat inactivation can be performed on the sample. In some embodiments, a processed/lysed sample can undergo heat inactivation to inactivate, in the lysed sample, the proteins used during lysing (e.g., a PK enzyme or a lysing reagent) and/or other residual proteins in the sample (e.g., RNases, DNases, viral proteins, etc.). In some cases, a heating element integrated into the nucleic acid detection device can be used for heat-inactivation. The heating element can be powered by a battery or another source of thermal or electric energy that is integrated with the nucleic acid detection device.

In certain instances, enzyme inactivation can be performed on the sample. In some embodiments, a processed/lysed sample can undergo enzyme inactivation to inhibit or inactivate, in the lysed sample, the proteins used during lysing (e.g., a PK enzyme or a lysing reagent) and/or other residual proteins in the sample (e.g., RNases, DNases, etc.). In some cases, a solution of reagents can be used to inactivate one or more enzymes present in the sample. Enzyme inactivation can occur before, during, or after lysis, when lysis is performed. For example, an RNase inhibitor may be included as a lysis reagent to inhibit native RNases within the sample (which might otherwise impair target and/or reporter detection downstream). Exemplary RNase inhibitors include RNAse Inhibitor, Murine (NEB), RnaseIn Plus (Promega), Protector Rnase Inhibitor (Roche), SuperaseIn (Ambion), RiboLock (Thermo), Ribosafe (Bioline), or the like. Alternatively, or in combination, when a protease is used for sample lysis, a protease inhibitor can be applied to the lysed sample to inactivate the protease prior to contacting the sample nucleic acids to the effector protein. Additional application of heat may not be required to inhibit the protease (e.g., proteinase K) sufficiently to prevent additional activity of the protease (which could potentially impair effector protein activity downstream, in some embodiments). Exemplary protease inhibitors include AEBSF, antipain, aprotinin, bestatin, chymostatin, EDTA, leupeptin, pepstatin A, phosphoramidon, PMSF, soybean trypsin inhibitor, TPCK, or the like. In some instances, enzyme inactivation may occur before, during, after, or instead of heat inactivation.

In some cases, a target nucleic acid within the sample can undergo amplification before binding to a guide nucleic acid, for example a crRNA of an effector protein. The target nucleic acid within a purified sample can be amplified. In some instances, amplification can be accomplished using loop mediated amplification (LAMP), isothermal recombinase polymerase amplification (RPA), and/or polymerase chain reaction (PCR). In some instances, digital droplet amplification can used. Such nucleic acid amplification of the sample can improve at least one of a sensitivity, specificity, or accuracy of the detection of the target RNA. The reagents for nucleic acid amplification can comprise a recombinase, an oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence-based amplification (NASBA), hinge-initiated primer-dependent amplification of nucleic acids (HIP), nicking enzyme amplification reaction (NEAR), or improved multiple displacement amplification (IMDA).

Additionally, target nucleic acid can optionally be amplified before binding to the guide nucleic acid (e.g., crRNA) of the effector protein. This amplification can be PCR amplification or isothermal amplification. This nucleic acid amplification of the sample can improve at least one of sensitivity, specificity, or accuracy of the detection the target RNA. The reagents for nucleic acid amplification can comprise a recombinase, a oligonucleotide primer, a single-stranded DNA binding (SSB) protein, and a polymerase. Such reagents are exemplary amplification reagents useful for the systems, compositions, methods, kits, devices, and solutions described herein. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA).

In some embodiments, the method further comprises actuating flow of the sample liquid through the heating channel to each of the reaction chambers. In some embodiments, the method of actuating comprises actuating using a plunger, a spring-actuated plunger, or a spring mechanism. In some embodiments, the actuation is manual. In some embodiments, actuation is configured to move the sample from the sample interface to the heating region via manual actuation of the first actuator. In some embodiments, the device is configured to be operated manually without electrical power. In some embodiments, actuation is achieved using a pneumatic pump, a sliding device, a rotary device, and/or a lateral flow device. In some embodiments, the method further comprises reacting the sample liquid with the effector protein, the guide nucleic acid, and the reporter. In some embodiments, the reagents described herein may include a composition for improving detection signal strength, detection reaction time, detection reaction efficiency, stability, solubility, or the like. In some embodiments, the reaction may generate a colorimetric signal, a fluorescent signal, an electrochemical signal, a chemiluminescent signal, or another type of signal. In some embodiments, the reaction may induce color-change in substances.

In some embodiments, the method further comprises detecting a detectable signal when a target nucleic acid is present in the sample. The method can further comprise using an effector protein-based detection module to detect one or more targets (e.g., target sequences or target nucleic acids) in the sample. In some cases, the sample can be divided into a plurality of aliquots or subsamples to facilitate sample preparation and to enhance the detection capabilities of the devices of the present disclosure. In some cases, the sample is not divided into subsamples. In some embodiments, the detectable signal is a colorimetric signal, a fluorescent signal, an electrochemical signal, a chemiluminescent signal, or another type of signal. In some embodiments, the detectable signal may be a color-change in substances. In some embodiments, detection is achieved using a sensor or detector. In some embodiments, detection is achieved either directly or indirectly. Additional illustrative embodiments for detecting a target nucleic acid using devices described herein are provided herein.

Further illustrative embodiments for devices (e.g., microfluidic devices) and methods of detecting target nucleic acids are described in further detail for example in WO2021207702, WO2021236850, WO2020142739, WO2021159020, WO2020028729, WO2020142754, WO2022133108, WO2020257356, WO2021243308, WO 2022061166, WO2022132833, and WO2022020393, the entire contents of each of which are incorporated herein by reference.

### X. Methods of Treating a Disease or Disorder

Described herein are methods for treating a disease in a subject by editing a target nucleic acid associated with a gene or expression of a gene related to the disease. In some embodiments, the methods comprise methods of editing nucleic acid described herein.

In some embodiments, methods for treating a disease in a subject comprises administration of a composition(s) or component(s) of a system described herein. In some embodiments, the composition(s) or component(s) of the system comprises use of a recombinant nucleic acid (DNA or RNA), administered for the purpose to edit a nucleic acid. In some embodiments, the composition or component of the system comprises use of a vector to introduce a functional gene or transgene. In some embodiments, vectors comprise nonviral vectors, including cationic polymers, cationic lipids, or bio-responsive polymers. In some embodiments, the bio-responsive polymer exploits chemical-physical properties of the endosomal environment (e.g., pH) to preferentially release the genetic material in the intracellular space. In some embodiments, vectors comprise viral vectors, including retroviruses, adenoviruses, adeno-associated viruses, and herpes simplex viruses. In some embodiments, the vector comprises a replication-defective viral vector, comprising an insertion of a therapeutic gene inserted in genes essential to the lytic cycle, preventing the virus from replicating and exerting cytotoxic effects. Methods of gene therapy that are applicable to the compositions and systems described herein are described in more detail in Ingusci et al., “Gene Therapy Tools for Brain Diseases”, Front. Pharmacol. 10:724 (2019), which is hereby incorporated by reference in its entirety.

In some embodiments, treating, preventing, or inhibiting disease or disorder in a subject may comprise contacting a target nucleic acid associated with a particular ailment with a composition described herein. In some aspects, the methods of treating, preventing, or inhibiting a disease or disorder may involve removing, editing, modifying, replacing, transposing, or affecting the regulation of a genomic sequence of a patient in need thereof. In some embodiments, the methods of treating, preventing, or inhibiting a disease or disorder may involve modulating gene expression.

Described herein are compositions and methods for treating a disease in a subject by editing a target nucleic acid associated with a gene or expression of a gene related to the disease. In some embodiments, methods comprise administering a composition or cell described herein to a subject. By way of non-limiting example, the disease may be a cancer, an ophthalmological disorder, a neurological disorder, a neurodegenerative disease, a blood disorder, or a metabolic disorder, or a combination thereof. The disease may be an inherited disorder, also referred to as a genetic disorder. The disease may be the result of an infection or associated with an infection. Also, by way of non-limiting example, the compositions are pharmaceutical compositions described herein.

The compositions and methods described herein may be used to treat, prevent, or inhibit a disease or syndrome in a subject. In some embodiments, the disease is a liver disease, a lung disease, an eye disease, or a muscle disease. Exemplary diseases and syndromes include but are not limited to the diseases and syndromes listed in TABLE 7.

### Diseases

Described herein are compositions and methods for treating a disease in a subject by modifying a target nucleic acid associated with a gene or expression of a gene related to the disease. In some embodiments, methods comprise administering a composition or cell described herein to a subject. By way of non-limiting example, the disease may be a cancer, an ophthalmological disorder, a neurological disorder, a neurodegenerative disease, a blood disorder, or a metabolic disorder, or a combination thereof. The disease may be an inherited disorder, also referred to as a genetic disorder. The disease may be the result of an infection or associated with an infection. The disease may be the result of or cause a syndrome.

A genetic disorder can be a genetic disease. A “genetic disease”, as used herein, can refer to a disease caused by one or more mutations in the DNA of an organism. In some embodiments, a disease is referred to as a “disorder.” Mutations may be due to several different cellular mechanisms, including, but not limited to, an error in DNA replication, recombination, or repair, or due to environmental factors. Mutations may be encoded in the sequence of a target nucleic acid from the germline of an organism. A genetic disease may comprise a single mutation, multiple mutations, or a chromosomal aberration.

The compositions and methods described herein may be used to treat, prevent, or inhibit a disease or syndrome in a subject. In some embodiments, the disease is a liver disease, a lung disease, an eye disease, or a muscle disease. Exemplary diseases and syndromes include, but are not limited to any one of the diseases or syndromes are set forth in TABLE 8.

In some embodiments, compositions and methods modify at least one gene associated with the disease or the expression thereof. In some embodiments, the disease is Alzheimer's disease and the gene is selected from APP, BACE-1, PSD95, MAPT, PSEN1, PSEN2, and APOEε4. In some embodiments, the disease is Parkinson's disease and the gene is selected from SNCA, GDNF, and LRRK2. In some embodiments, the disease comprises Centronuclear myopathy and the gene is DNM2. In some embodiments, the disease is Huntington's disease and the gene is HTT. In some embodiments, the disease is Alpha-1 antitrypsin deficiency (AATD) and the gene is SERPINA1. In some embodiments, the disease is amyotrophic lateral sclerosis (ALS) and the gene is selected from SOD1, FUS, C9ORF72, ATXN2, TARDBP, and CHCHD10. In some embodiments, the disease comprises Alexander Disease and the gene is GFAP. In some embodiments, the disease comprises anaplastic large cell lymphoma and the gene is CD30. In some embodiments, the disease comprises Angelman Syndrome and the gene is UBE3A. In some embodiments, the disease comprises Calcific Aortic Stenosis and the gene is Apo(a). In some embodiments, the disease comprises CD3Z-associated primary T-cell immunodeficiency and the gene is CD3Z or CD247. In some embodiments, the disease comprises CD18 deficiency and the gene is ITGB2. In some embodiments, the disease comprises CD40L deficiency and the gene is CD40L. In some embodiments, the disease comprises CNS trauma and the gene is VEGF. In some embodiments, the disease comprises coronary heart disease and the gene is selected from FGA, FGB, and FGG. In some embodiments, the disease comprises MECP2 Duplication syndrome and Rett syndrome and the gene is MECP2. In some embodiments, the disease comprises a bleeding disorder (coagulation) and the gene is FXI. In some embodiments, the disease comprises fragile X syndrome and the gene is FMR1. In some embodiments, the disease comprises Fuchs Corneal Dystrophy and the gene is selected from ZEB1, SLC4A11, and LOXHD1. In some embodiments, the disease comprises GM2-Gangliosidoses (e.g., Tay Sachs Disease, Sandhoff disease) and the gene is selected from HEXA and HEXB. In some embodiments, the disease comprises Hearing loss disorders and the gene is DFNA36. In some embodiments, the disease is Pompe disease, including infantile onset Pompe Disease (IOPD) and late onset Pompe Disease (LOPD) and the gene is GAA. In some embodiments, the disease is Retinitis pigmentosa and the gene is selected from PDE6B, RHO, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROM1, KLHL7, CNGB1, TTC8, ARL6, DHDDS, BEST1, LRAT, SPARA7, CRX, CLRN1, RPE65, and WDR19. In some embodiments, the disease comprises Leber Congenital Amaurosis Type 10 and the gene is CEP290. In some embodiments, the disease is cardiovascular disease and/or lipodystrophies and the gene is selected from ABCG5, ABCG8, AGT, ANGPTL3, APOCIII, APOA1, APOL1, ARH, CDKN2B, CFB, CXCL12, FXI, FXII, GATA-4, MIA3, MKL2, MTHFD1L, MYH7, NKX2-5, NOTCH1, PKK, PCSK9, PSRC1, SMAD3, and TTR. In some embodiments, the disease comprises acromegaly and the gene is GHR. In some embodiments, the disease comprises acute myeloid leukemia and the gene is CD22. In some embodiments, the disease is diabetes and the gene is GCGR. In some embodiments, the disease is NAFLD/NASH and the gene is selected from DGAT2 and PNPLA3. In some embodiments, the disease is cancer and the gene is selected from STAT3, YAP1, FOXP3, AR (Prostate cancer), and IRF4 (multiple myeloma). In some embodiments, the disease is cystic fibrosis and the gene is CFTR. In some embodiments, the disease is Duchenne Muscular Dystrophy and the gene is DMD. In some embodiments, the disease comprises angioedema and the gene is PKK. In some embodiments, the disease comprises thalassemia and the gene is TMPRSS6. In some embodiments, the disease comprises achondroplasia and the gene is FGFR3. In some embodiments, the disease comprises Cri du chat syndrome and the gene is selected from CTNND2. In some embodiments, the disease comprises sickle cell anemia and the gene is Beta globin gene. In some embodiments, the disease comprises Alagille Syndrome and the gene is selected from JAG1 and NOTCH2. In some embodiments, the disease comprises Charcot Marie Tooth Disease and the gene is selected from PMP22 and MFN2. In some embodiments, the disease comprises Crouzon syndrome and the gene is selected from FGFR2, FGFR3, and FGFR3. In some embodiments, the disease comprises Dravet Syndrome and the gene is selected from SCN1A and SCN2A. In some embodiments, the disease comprises Emery-Dreifuss syndrome and the gene is selected from EMD, LMNA, SYNE1, SYNE2, FHL1, and TMEM43. In some embodiments, the disease comprises Factor V Leiden Thrombophilia and the gene is F5. In some embodiments, the disease comprises Fanconi anemia and the gene is selected from FANCA, FANCB, FANCC, FANCD1, FANCD2, FANCE, FANCF, FANCG, FANCI, FANCJ, FANCL, FANCM, FANCN, FANCP, FANCS, RAD51C, and XPF. In some embodiments, the disease comprises Familial Creutzfeld-Jakob Disease and the gene is PRNP. In some embodiments, the disease comprises Familial Mediterranean Fever and the gene is MEFV. In some embodiments, the disease comprises Friedreich's ataxia and the gene is FXN. In some embodiments, the disease comprises Gaucher disease and the gene is GBA. In some embodiments, the disease comprises human papilloma virus (HPV) infection and the gene is HPV E7. In some embodiments, the disease comprises hemochromatosis and the gene is HFE, optionally comprising a C282Y mutation. In some embodiments, the disease comprises Hemophilia A and the gene is FVIII. In some embodiments, the disease comprises histiocytosis and the gene is CD1. In some embodiments, the disease comprises immunodeficiency 17 and the gene is CD3D. In some embodiments, the disease comprises immunodeficiency 13 and the gene is CD4. In some embodiments, the disease comprises Common Variable Immunodeficiency and the gene is selected from CD19 and CD81. In some embodiments, the disease comprises Joubert syndrome and the gene is selected from INPP5E, TMEM216, AHI1, NPHP1, CEP290, TMEM67, RPGRIP1L, ARL13B, CC2D2A, OFD1, TMEM138, TCTN3, ZNF423, and AMRC9. In some embodiments, the disease comprises leukocyte adhesion deficiency and the gene is CD18. In some embodiments, the disease comprises Li-Fraumeni syndrome and the gene is TP53. In some embodiments, the disease comprises lymphoproliferative syndrome and the gene is CD27. In some embodiments, the disease comprises Lynch syndrome and the gene is selected from MSH2, MLH1, MSH6, PMS2, PMS1, TGFBR2, and MLH3. In some embodiments, the disease comprises mantle cell lymphoma and the gene is CD5. In some embodiments, the disease comprises Marfan syndrome and the gene is FBN1. In some embodiments, the disease comprises mastocytosis and the gene is CD2. In some embodiments, the disease comprises methylmalonic acidemia and the gene is selected from MMAA, MMAB, and MUT. In some embodiments, the disease is mycosis fungoides and the gene is CD7. In some embodiments, the disease is myotonic dystrophy and the gene is selected from CNBP and DMPK. In some embodiments, the disease comprises neurofibromatosis and the gene is selected from NF1, and NF2. In some embodiments, the disease comprises osteogenesis imperfecta and the gene is selected from COLIA1, COLIA2, and IFITM5. In some embodiments, the disease is non-small cell lung cancer and the gene is selected from KRAS, EGFR, ALK, METex14, BRAF V600E, ROS1, RET, and NTRK. In some embodiments, the disease comprises Peutz-Jeghers syndrome and the gene is STK11. In some embodiments, the disease comprises polycystic kidney disease and the gene is selected from PKD1 and PKD2. In some embodiments, the disease comprises Severe Combined Immune Deficiency and the gene is selected from IL7R, RAG1, JAK3. In some embodiments, the disease comprises PRKAG2 cardiac syndrome and the gene is PRKAG2. In some embodiments, the disease comprises Spinocerebellar ataxia and the gene is selected from ATXN1, ATXN2, ATXN3, PLEKHG4, SPTBN2, CACNA1A, ATXN7, ATXN8OS, ATXN10, TTBK2, PPP2R2B, KCNC3, PRKCG, ITPR1, TBP, KCND3, and FGF14. In some embodiments, the disease comprises Usher Syndrome and the gene is selected from MYO7A, USH1C, CDH23, PCDH15, USH1G, USH2A, GPR98, DFNB31, and CLRN1. In some embodiments, the disease comprises von Willebrand disease and the gene is VWF. In some embodiments, the disease comprises Waardenburg syndrome and the gene is selected from PAX3, MITF, WS2B, WS2C, SNAI2, EDNRB, EDN3, and SOX10. In some embodiments, the disease comprises Wiskott-Aldrich Syndrome and the gene is WAS. In some embodiments, the disease comprises von Hippel-Lindau disease and the gene is VHL. In some embodiments, the disease comprises Wilson disease and the gene is ATP7B. In some embodiments, the disease comprises Zellweger syndrome and the gene is selected from PEX1, PEX2, PEX3, PEX5, PEX6, PEX10, PEX12, PEX13, PEX14, PEX16, PEX19, and PEX26. In some embodiments, the disease comprises infantile myofibromatosis and the gene is CD34. In some embodiments, the disease comprises platelet glycoprotein IV deficiency and the gene is CD36. In some embodiments, the disease comprises immunodeficiency with hyper-IgM type 3 and the gene is CD40. In some embodiments, the disease comprises hemolytic uremic syndrome and the gene is CD46. In some embodiments, the disease comprises complement hyperactivation, angiopathic thrombosis, or protein-losing enteropathy and the gene is CD55. In some embodiments, the disease comprises hemolytic anemia and the gene is CD59. In some embodiments, the disease comprises calcification of joints and arteries and the gene is CD73. In some embodiments, the disease comprises immunoglobulin alpha deficiency and the gene is CD79A. In some embodiments, the disease comprises C syndrome and the gene is CD96. In some embodiments, the disease comprises hairy cell leukemia and the gene is CD123. In some embodiments, the disease comprises histiocytic sarcoma and the gene is CD163. In some embodiments, the disease comprises autosomal dominant deafness and the gene is CD164. In some embodiments, the disease comprises immunodeficiency 25 and the gene is CD247. In some embodiments, the disease comprises methymalonic acidemia due to transcobalamin receptor defect and the gene is CD320.

### Cancer

In some embodiments, the disease is cancer. Non-limiting examples of cancers include: acute lymphoblastic leukemia; acute lymphoblastic lymphoma; acute lymphocytic leukemia; acute myelogenous leukemia; acute myeloid leukemia (adult/childhood); adrenocortical carcinoma; AIDS-related cancers; AIDS-related lymphoma; anal cancer; appendix cancer; astrocytoma; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; Ewing sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; Hodgkin's lymphoma; hypopharyngeal cancer; hypothalamic and visual pathway glioma; intraocular melanoma; islet cell carcinoma (endocrine pancreas); Kaposi Sarcoma; kidney cancer (renal cell cancer); Langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenström; male breast cancer; malignant fibrous histiocytoma of bone/osteosarcoma; medulloblastoma; medulloepithelioma; melanoma; melanoma, intraocular (eye); Merkel cell cancer; Merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-Hodgkin's lymphoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; NUT midline carcinoma; retinoblastoma; rhabdomyosarcoma, childhood; salivary gland cancer; sarcoma, Ewing family of tumors; Sézary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sézary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In some embodiments, the cancer is a solid cancer (i.e., a tumor). In some embodiments, the cancer is selected from a blood cell cancer, a leukemia, and a lymphoma. The cancer can be a leukemia, such as, by way of non-limiting example, acute myeloid (or myelogenous) leukemia (AML), chronic myeloid (or myelogenous) leukemia (CML), acute lymphocytic (or lymphoblastic) leukemia (ALL), and chronic lymphocytic leukemia (CLL). In some embodiments, the cancer is any one of colon cancer, rectal cancer, renal-cell carcinoma, liver cancer, bladder cancer, cancer of the kidney or ureter, lung cancer, non-small cell lung cancer, cancer of the small intestine, esophageal cancer, melanoma, bone cancer, pancreatic cancer, skin cancer, brain cancer (e.g., glioblastoma), cancer of the head or neck, melanoma, uterine cancer, ovarian cancer, breast cancer, testicular cancer, cervical cancer, stomach cancer, Hodgkin's Disease, non-Hodgkin's lymphoma, and thyroid cancer.

In some embodiments, mutations are associated with cancer or are causative of cancer. The target nucleic acid, in some embodiments, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, a gene associated with cell cycle, or a combination thereof. Non-limiting examples of genes comprising a mutation associated with cancer are ABL, ACE, AF4/HRX, AKT-2, ALK, ALK/NPM, AML1, AML1/MTG8, APC, ATM, AXIN2, AXL, BAPI, BARD1, BCL-2, BCL-3, BCL-6, BCR/ABL, BLM, BMPR1A, BRCA1, BRCA2, BRIP1, c-MYC, CASR, CCR5, CDC73, CDH1, CDK4, CDKN1B, CDKN1C, CDKN2A, CEBPA, CHEK2, CREBBP, CTNNA1, DBL, DEK/CAN, DICER1, DIS3L2, E2A/PBX1, EGFR, ENL/HRX, EPCAM, ERG/TLS, ERBB, ERBB-2, ETS-1, EWS/FLI-1, FH, FKRP, FLCN, FMS, FOS, FPS, GATA2, GCG, GLI, GPC3, GPGSP, GREM1, HER2/neu, HOX11, HOXB13, HRAS, HST, IL-3, INT-2, JAK1, JUN, KIT, KS3, K-SAM, LBC, LCK, LMO1, LMO2, L-MYC, LYL-1, LYT-10, LYT-10/Cα1, MAS, MAX, MDM-2, MEN1, MET, MITF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PCDC1, PDGFRA, PHOX2B, PIM-1, PMS2, POLDI, POLE, POT1, PPARG, PRAD-1, PRKAR1A, PTCH1, PTEN, RAD50, RAD51C, RAD51D, RAF, RAR/PML, RAS-H, RAS-K, RAS-N, RB1, RECQL4, REL/NRG, RET, RHOM1, RHOM2, ROS, RUNX1, SDHA, SDHAF, SDHAF2, SDHB, SDHC, SDHD, SET/CAN, SIS, SKI, SMAD4, SMARCA4, SMARCB1, SMARCE1, SRC, STK11, SUFU, TAL1, TAL2, TAN-1, TIAM1, TERC, TERT, TIMP3, TMEM127, TNF, TP53, TRAC, TSC1, TSC2, TRK, VHL, WRN, and WT1. Non-limiting examples of oncogenes are KRAS, NRAS, BRAF, MYC, CTNNB1, and EGFR. In some embodiments, the oncogene is a gene that encodes a cyclin dependent kinase (CDK). Non-limiting examples of CDKs are Cdk1, Cdk4, Cdk5, Cdk7, Cdk8, Cdk9, Cdk11 and Cdk20. Non-limiting examples of tumor suppressor genes are TP53, RB1, and PTEN.

### Infections

Described herein are compositions and methods for treating an infection in a subject. Infections may be caused by a pathogen, e.g., bacteria, viruses, fungi, and parasites. Compositions and methods may modify a target nucleic acid associated with the pathogen or parasite causing the infection. In some embodiments, the target nucleic acid may be in the pathogen or parasite itself or in a cell, tissue or organ of the subject that the pathogen or parasite infects. In some embodiments, the methods described herein include treating an infection caused by one or more bacterial pathogens. Non-limiting examples of bacterial pathogens include Acholeplasma laidlawii, Brucella abortus, Chlamydia psittaci, Chlamydia trachomatis, Cryptococcus neoformans, Escherichia coli, Legionella pneumophila, Lyme disease spirochetes, methicillin-resistant Staphylococcus aureus, Mycobacterium leprae, Mycobacterium tuberculosis, Mycoplasma arginini, Mycoplasma arthritidis, Mycoplasma genitalium, Mycoplasma hyorhinis, Mycoplasma orale, Mycoplasma pneumoniae, Mycoplasma salivarium, Neisseria gonorrhoeae, Neisseria meningitidis, Pneumococcus, Pseudomonas aeruginosa, sexually transmitted infection, Streptococcus agalactiae, Streptococcus pyogenes, and Treponema pallidum.

In some embodiments, methods described herein include treating an infection caused by one or more viral pathogens. Non-limiting examples of viral pathogens include adenovirus, blue tongue virus, chikungunya, coronavirus (e.g., SARS-CoV-2), cytomegalovirus, Dengue virus, Ebola, Epstein-Barr virus, feline leukemia virus, Hemophilus influenzae B, Hepatitis Virus A, Hepatitis Virus B, Hepatitis Virus C, herpes simplex virus I, herpes simplex virus II, human papillomavirus (HPV) including HPV16 and HPV18, human serum parvo-like virus, human T-cell leukemia viruses, immunodeficiency virus (e.g., HIV), influenza virus, lymphocytic choriomeningitis virus, measles virus, mouse mammary tumor virus, mumps virus, murine leukemia virus, polio virus, rabies virus, Reovirus, respiratory syncytial virus (RSV), rubella virus, Sendai virus, simian virus 40, Sindbis virus, varicella-zoster virus, vesicular stomatitis virus, wart virus, West Nile virus, yellow fever virus, or any combination thereof.

In some embodiments, methods described herein include treating an infection caused by one or more parasites. Non-limiting examples of parasites include helminths, annelids, platyhelminthes, nematodes, and thorny-headed worms. In some embodiments, parasitic pathogens comprise, without limitation, Babesia bovis, Echinococcus granulosus, Eimeria tenella, Leishmania tropica, Mesocestoides corti, Onchocerca volvulus, Plasmodium falciparum, Plasmodium vivax, Schistosoma japonicum, Schistosoma mansoni, Schistosoma spp., Taenia hydatigena, Taenia ovis, Taenia saginata, Theileria parva, Toxoplasma gondii, Toxoplasma spp., Trichinella spiralis, Trichomonas vaginalis, Trypanosoma brucei, Trypanosoma cruzi, Trypanosoma rangeli, Trypanosoma rhodesiense, Balantidium coli, Entamoeba histolytica, Giardia spp., Isospora spp., Trichomonas spp., or any combination thereof.

### Sequences and Tables

TABLE 1 provides illustrative amino acid sequences of effector proteins that are useful in the compositions, systems and methods described herein.

TABLE 1
Effector Proteins

| SEQ ID | Protein | |
|---|---|---|
| NO: | ID NO | Effector Protein Amino Acid Sequence |
| 1 | 273656 | MKKSIFDQFVNQYALSKTLRFELKPVGETGRMLEEAKVFAKDETIKKKYEATKPFFNKLHREFVEEALNEVE |
| | | LAGLPEYFEIFKYWKRYKKKFEKDLQKKEKELRKSVVGFFNAQAKEWAKKYETLGVKKKDVGLLFEENVF |
| | | AILKERYGNEEGSQIVDESTGKDVSIFDSWKGFTGYFIKFQETRKNFYKDDGTATALATRIIDQNLKRFCDNL |
| | | LIFESIRDKIDFSEVEQTMGNSIDKVFSVIFYSSCLLQEGIDFYNCVLGGETLPNGEKRQGINELINLYRQKTSEK |
| | | VPFLKLLDKQILSEKEKFMDEIENDEALLDTLKIFRKSAEEKTTLLKNIFGDFVMNQGKYDLAQIYISRESLNTI |
| | | SRKWTSETDIFEDSLYEVLKKSKIVSASVKKKDGGYAFPEFIALIYVKSALEQIPTEKFWKERYYKNIGDVLN |
| | | KGFLNGKEGVWLQFLLIFDFEFNSLFEREIIDENGDKKVAGYNLFAKGFDDLLNNFKYDQKAKVVIKDFADE |
| | | VLHIYQMGKYFAIEKKRSWLADYDIDSFYTDPEKGYLKFYENAYEEIIQVYNKLRNYLTKKPYSEDKWKLNF |
| | | ENPTLADGWDKNKEADNSTVILKKDGRYYLGLMARGRNKLFDDRNLPKILEGVENGKYEKVVYKYFPDQA |
| | | KMFPKVCFSTKGLEFFQPSEEVITIYKNSEFKKGYTFNVRSMQRLIDFYKDCLVRYEGWQCYDFRNLRKTED |
| | | YRKNIEEFFSDVAMDGYKISFQDVSESYIKEKNQNGDLYLFEIKNKDWNEGANGKKNLHTIYFESLFSADNIA |
| | | MNFPVKLNGQAEIFYRPRTEGLEKERIITKKGNVLEKGDKAFHKRRYTENKVFFHVPITLNRTKKNPFQFNAK |
| | | INDFLAKNSDINVIGVDRGEKQLAYFSVISQRGKILDRGSLNVINGVNYAEKLEEKARGREQARKDWQQIEGI |
| | | KDLKKGYISQVVRKLADLAIQYNAIIVFEDLNMRFKQIRGGIEKSVYQQLEKALIDKLTFLVEKEEKDVEKAG |
| | | HLLKAYQLAAPFETFQKMGKQTGIVFYTQAAYTSRIDPVTGWRPHLYLKYSSAEKAKADLLKFKKIKFVDG |
| | | RFEFTYDIKSFREQKEHPKATVWTVCSCVERFRWNRYLNSNKGGYDHYSDVTKFLVELFQEYGIDFERGDIV |
| | | GQIEVLETKGNEKFFKNFVFFFNLICQIRNTNASELAKKDGKDDFILSPVEPFFDSRNSEKFGEDLPKNGDDNG |
| | | AFNIARKGLVIMDKITKFADENGGCEKMKWGDLYVSNVEWDNFVANK |
| 2 | 286498 | MKSIFDNFTGLYSLSKTLRFELRPVGQTLENIKNGHFLESDKKMADDYQDVKKIIDNYHKFFIDDVLKGASFD |
| | | WALLEKELTDFNKNKTDDSKVEAEQKKLREQIAKTLAGDKRFKSLTASTPNDLFNKDKDFIGWLEQSSVKEI |
| | | RKDALDTFKKFSSYFKGFQENRKNVYSADDIPTAVPYRIVNDNFPKFLQNISIFKTIQEKCPQVIADVENELAS |
| | | YLGKEKLADIFTVQAFNKYLCQGGKENQRGIDFYNQVIGGIAEKEGGVNLRGINQFLNLYWQQHPDFAKEN |
| | | RRIKMVPLYKQILSDRSSLSFKIETIDTDEELKTAISEYADKLESKSNDEKKSVLDVCVELFDSIKEQNLQEIYV |
| | | NRKDINNISRILTGDWSWLQSRMNLYADEVFTTKAEKTRWQKSVDGDEGENKSKGAYSLAELNRVLEYASE |
| | | NVAETDIRITDYFCHRNRFYYEKESGLFKQGEELVALSIKESCEDILSKRKAMNEAFANISESNSLRDNSEDIA |
| | | KTKTYLDSVQDLLHRIKPLKVNGLGDPSFYAVFDSIYSALSEVISIYNKTRNYITRKAESPEKYKLNFDNPTLA |
| | | NGWDLNKEKDNTCVLLRKNGMYYLGIMNPKDKPKFAEKYDCGTESCYEKMIYKLLPGPNKMLPKVFFSTK |
| | | GKKQYNPPENILHGYEQGKHKKGVAFDINFCHELIDWFKSAINQHEDWKKFGFKFSDTKSYKDISDFYREVT |
| | | EQGYKLTFINIPESEISKMVSEGKLYLFQIYNMDFAPGANGMPNMHTLYWKNLFSEENLKDVVLKLNGEAEL |
| | | FYRPAGIKEPVVHAKGSYLVNRITKDGEPIPEKIHDEIYRNANGNLNNLSKEASEYKESHKVVIKQASHEIIKD |
| | | RHYTEPKFLFHIPLTINFKAPSMKPTVCTAIQNENVRRFLKNNPDVNIIGLDRGERNLIYLSLINQKGEIIKQFSF |
| | | NDVEREQNGQTVKVNYHEKLDQREKERDAARKSWQAIGKIAELKEGYLSAVIHQLTKLMVEYNAIVVMED |
| | | LNFGFKRGRFHVEKQVYQKFEHMLIDKLNYLVFKDRGLNEPGGALNGYQLAGQFESFQKLGKQSGMLFYV |
| | | PAAYTSKIDPKTGFVSMMNFKDLTNVHKKRDFFSKFEDIHFDEATCSFVFTFDYKNFNGKAKEEMKQTKWA |
| | | VYSREKRIVYFSKTKSSEDIMPTEKLRALFESDGIEYKSGNNIHDSVMAVGADLKEGAKPSKEIADFWDGLLY |
| | | NFKLILQMRNSNAKTGEDYIISPVMASDGTFFDSRVEAKKGKDAKLPLDADANGAYHIALKGLSLINKINLA |
| | | GEDELKKFDMKISNEDWFNFAQEKKYAE |
| 3 | 279793 | MTKQNKSVTQNTKRKNFGEFTNLYSLSKTLRFELKPVKATKTILEVERKDRENFKKDRKIAKNYQKLKGILN |
| | | ELHQEFIQDVMREFSFQEKEIKEFEERYLEALNFKEKDNYKKRTQLKNAYEKVAKKLAGKIATAFGKYNQEK |
| | | YGVKFTKKNLTGENVFDILEGKYKGDKKILGIIHTFKFKPTKEEKKQGKEAVNFSTYLGGFNQNRENFYKGE |
| | | MKAGQFATRTIENLIQFLKNKKLFIDKYKDNYQKIGFSQEQVEIFNLNYFNNLFLQEGLDVYNGILGAKKGEK |
| | | NTENDGLNQKINLFKQKEKTRCKANGEKFNKSDYPIFKELYKQIGSIKKDNDVYVEIKSDEELVNVLQSLPEK |
| | | TANTLREVQKFYENFFDKIFNDEFDLDKIYLPKSVGTHFSHLAFSDWSKLAFVFNKRWRNEKVKIKEGEDVN |
| | | VQSRSLADIKKRMEEILEMDGGVSFGKTYCQKVGLEKEARTIEDVWSGFWKIIQYHINSQFIGGEKEVFDKEK |
| | | KDDKTEKIQTIDDLQEEYLQATEMYRERMVESEEGLNDGEEKEIKTKLKNYLDRIKDIERIARYFDLRKHFDD |
| | | IDEASKDGDFYFIYQELLQDISEAKINDHYNEIRNYLTKANVVDDKFKLNFNDGQTLSGWDLNKETEKFSLIF |
| | | KRKVDGGVEYYLGIINKEKNKTIFDKKKHPEIFTENSEFEKMEYKLFPSPSKMLPKIAFTKNKEGERIKPVFLD |
| | | ENAGKEIAQIKKEFALFQDAKKEDKNKWSDEFDRKKLNKLIDYYKLVLEKHPEKYMQTFNFVFKSSAKYKN |
| | | LGEFNDDVARQNYVTKFVSVDKDYIDQKVESGELYLFKIHNKDWNLTKAGDTKKQSKKNLHTIYFEELFSE |
| | | KNIAEPVFKLSGGAEVFFRDAIEKKKQKKKKDKKGKEILEKFRFTKNKILFHVPITINYGKPSINQGQFNQKIN |
| | | EFIADNSRSVNILGIDRGEKHLLYYSLVDNNGKIIKSGSLNEINGVDYHEKLDKAEKERQEARKSWQKINQIK |
| | | NLKAGYISQVIKKIVDLAIENNAIIVLEDLNFGFKSFRQKIEKNVYQQFEKALIDKLGFVTDKEKLNHRQAPQL |
| | | SAPFESFEKMGKQTGIVFYVLATNTSKVCPQCQWKKNIFPHYSTKKSIAENLQKQYKMKMYWRENENRFEF |
| | | EYKGDGDKEFSSIFSNVDRVRYDKRANNNQGGYVIYQIDSTTKEKDGRNIKEKSITNLLKELLLEKFEIDNLE |
| | | GELLVKLSEKSPDVSKETIKDFFGLLNSILNIRNSMTDTEEDYIQCPACGFDTRKENKIGIKNGDDNGAYNIAL |
| | | RGRFLIERIKKAKKEDKKPNLTFSNNDYFQWVREFVK |
| 4 | 288512 | MFNNFIKKYSLQKTLRFELKPVGETADYIEDFKSEYLKDTVLKDEQRAKDYQEIKTLIDDYHREYIEECLREP |
| | | VDKKTGEILDFTQDLEDAFSYYQKLKENPTENRVGWEKEQESLRKKLVTSFVGNDGLFKKEFITRDLPEWLQ |
| | | KKGLWGEYKDTVENFKKFTTYFSGFHENRKNMYTAEAQSTAIANRLMNDNLPKFFNNYLAYQTIKEKHPDL |
| | | VFRLDDALLQAAGVEHLDEAFQPRYFSRLFAQSGITAFNELIGGRTTENGEKIQGLNEQINLYRQQNPEKAKG |
| | | FPRFMPLFKQILSDRETHSFLPDAFENDKELLQALRDYVDAATSEEGMISQLNKAMNQFVTADLKRVYIKSA |
| | | ALTSLSQELFHFFGVISDAIAWYAEKRLSPKKAQESFLKQEVYAIEELNQAVVGYIDQLEDQSELQQLLVDLP |
| | | DPQKPVSSFILTHWQKSQEPLQAVIAKVEPLFELEELSKNKRAPKHDKDQGGEGFQQVDAIKNMLDAFMEVS |
| | | HAIKPLYLVKGRKAIDMPDVDTGFYADFAEAYSAYEQVTVSLYNKTRNHLSKKPFSKDKIKINFDAPTLLNG |
| | | WDLNKESDNKSIILRKDGNFYLAIMHPKHTKVFDCYSASEAAGKCYEKMNYKLLSGANKMLPKVFFSKKGI |
| | | ETFSPPQEILDLYKNNEHKKGATFKLESCHKLIDFFKRNIPKYKVHPTDNFGWDVFGFHFSPTSSYGDLSGFYR |
| | | EVEAQGYKLWFSDVSEAYINKCVEEGKLFLFQIYNKDFSPNSTGKPNLHTLYWKGLFEPENLKDVVLKLNGE |
| | | AEIFYRKHSIKHEDKTIHRAKDPIANKNADNPKKQSVFDYDIIKDKRYTQDKFFFHVPISLNFKSQGVVRFND |
| | | KINGLLAAQDDVHVIGIDRGERHLLYYTVVNGKGEVVEQGSLNQVATDQGYVVDYQQKLHAKEKERDQAR |
| | | KNWSTIENIKELKAGYLSQVVHKLAQLIVKHNAIVCLEDLNFGFKRGRFKVEKQVYQKFEKALIDKLNYLVF |
| | | KERGATQAGGYLNAYQLAAPFESFEKLGKQTGILYYVRSDYTSKIDPATGFVDFLKPKYESMAKSKVFFESF |
| | | ERIQWNQAKGYFEFEFDYKKMCPSRKFGDYRTRWVVCTFGDTRYQNRRNKSSGQWETETIDVTAQLKALF |
| | | AAYGITYNQEDNIKDAIAAVKYTKFYKQLYWLLRLTLSLRHSVTGTDEDFILSPVADENGVFFDSRKATDKQ |
| | | PKDADANGAYHIALKGLWNLQQIRQHDWNVEKPKKLNLAMKNEEWFGFAQKKKFRA |
| 5 | 1895859 | MIKSYDDFTKLYSLQKTLRFELKPIGKTLEHIKKSEIIESDETLEEQYAIVKNIIDRFHRKHIDEALSLVDFTKHL |
| | | DTFKTIQELYLKRGKTDREKKELEELSADLRKLIVSYLKGNVKQKTQHNLNPIKERFEILFGKELFTNEEFFTL |
| | | AENKEEKKAIQAFKGFTTYFKGFQENRKNMYSEEDKSTAIAYRIINENMPLFIENIARFQKVLDVIEKTKLTEL |
| | | KQNLKTELKGHSVSDIFRIEYFNNVLTQEGISRYNTIIGGKTTTEGVKIQGLNEIINLHNQQSKDVKLPILKPLH |
| | | KQILSEEYSTSFKIKAFENDNEVLKAIDTFWNEHIEKSIHPVTGKRFNILLKIENLCKKLEKYKDKEIEKLFIERK |
| | | NLSTVSHQVYGQWNIIRDALRMHLEMNNKNIKEKDIDKYLDNDAFAWKEIKDSIKIYKEHVEDAKELDENGI |
| | | VKYFSSMSINEEDDEKEYSISLIKNINEKYNNVKSILEEDRTGKSDLHQDKEKVAIIKEFLDSLKQLQWFLKLL |
| | | YVTVPLDEKDYEFYNELEVYYEALLPLNSLYNKVRNYMTRKPYSVEKFKLNFYSPTLLDGWDKNKETANLS |
| | | IILKKNGKYYLGIMNKENNTIFENFPKSKSNDYYEKMIYKLLPGPNKMLPKVFFSKKGLEYYKPSKEILRIYEK |
| | | GEFKKDKSGNFKKESLHTLIDFYKEAIAKNEDWKIFKFKFKNTREYEDISQFYRDVEEQGYLIIFEKVDANYV |
| | | DKLVEEGELYLFQIYNKDFSENKKSKGNPNLHTIYWESLFDNQNLKDVVYKLNGEAEVFYRKKSIDYPEEIY |
| | | NNGHHKEELNGKFNYPIIKDRRYTQDKFLFHVPITMNFISKEEKRVNQLACEYLSTTKEDVHIIGIDRGERHLL |
| | | YLSLIDKEGNIKKQLSLNTIKNENYDKEIDYRVKLDEKEKKRDEARKNWDVIENIKELKEGYMSQVIHIIAKM |
| | | MAEEKAILIMEDLNIGFKRGRFKVEKQVYQKFEKMMIDKLNYLVFKNKEPLEPGGSLNAYQLTSKFDSFKKL |
| | | GKQSGFIFYVPSAYTSKIDPTTGFYNFIRVDVPNLEKGKEFFSKFEKIIYNTKEDYFEFHCKYGKFVPEPKNKD |
| | | NDRKTKESLTYYNAIKDTVWVVCSTHHERYKIVRNKAGYYESQPVDVTKNLRDIFSEANINYSDGKDIKPIII |
| | | ESNNAKLLKSIAEQLKLILAMRYNNGKHDDDEKDYILSPVKNKQGKFFCSLDGDQSLPINADANGAYNIALK |
| | | GLLLIEKIKKQQGKAKDLYISNLEWFMFMMSR |
| 6 | 1895868 | MRTTTSLDAFTNRYALSKTLRFELKPIGNTQMMLEQNNVFAKDRAIREKYEKTKPWIDLLHREFVAESLQNA |
| | | QLGNLDDYYAALQNVQKITKDTNAEDKKRWKKGFEKQEKRLRKEVVALFDKAAHIWATQRYPQLKKKTK |
| | | DFLFEEGVFEHVLFARYGSAPDTTVKIVTSNPETGEVIDEREESIFKGWKGFTGYFDKFFETRKNFYKDNGTA |
| | | TAIATRAINQNLRRFAENMQKLTDIKNNYPELLAHTDFGDFDIAHAQSLDFYARTCLLQEGIDAYNKKFVGV |
| | | LKSAINEYQQKNKGVRISYPKTLDNQILGERERRLFDVIEDDRELHDVFRAFVDDGTVFAAEMRQLAQAFSA |
| | | QNGTYDYTQIYISKKGFETISRKYTHDTRAWHDALADVFKAKAKKRIATTASGEKKFPAYIPVAYITQALTLV |
| | | QESEDTECTWKERYASITENKTLEEGFFAIFADEFERLFVHMEATVQDTDYVVAEDKAKKLLSDGQITKNEQ |
| | | TTQIIKEYADALLRIYQMAKYFAVEKKSMWDDAVAIDDTFYETFKEIYGNTHSTIVASYNLLRNYLTKKPWE |
| | | DVQKWKLNFENPTLLDGWDKNKEAANFGVILRDGDKFYLGIMRKGHNNIFANQHHSNFEGQGLQKMVYK |
| | | FFPDPKKMFPKVCFSAKGMEFFAPSEEIVRIYKNAEFKSGDTFNVESMQKLIDFYKNALQKYDGWKIYDFKH |
| | | LKDTAQYTSNIGEFYDDVAKGGYQLGWQNISKEYVEEKNANGELYLFQIKNKDWNDGATGRKNLHTLYFE |
| | | YLFSEKNAAADFVFRLNGGAEVFYRPAAIESKTERRGNREVAAKKRYTQDKVFLHVPITLNRTAGDVKTSAF |
| | | NDAVNRFLAGNPDINIMGIDRGEKHLAYYSIIDQNGNRIVSGSFNTIGSKDYHALLTERQGAREEARKNWQR |
| | | VEQIKDLKKGYISLVVREIADLAIKHNAIIVLENLNMRFKQIRGGIEKSVYQQLEKALIEKLNFLVNKGEVDAT |
| | | KAGHLLRAYQLAAPFETFEKMGNQTGIIFYTTASYTSQVDPVTGWRPHVYLKYRNAQKTKEDILRIFDDIVF |
| | | NDEKQRFEFAYRHNGVSWTVCSSVERHRWNRSNNAGKGGYDVFPVEGEGSITQRLQEACAQRGIDTTRNIL |
| | | AQIDELDESASATVSFLRDLCFYFRLICQIRNTDDGADDINAQDFLMSPVEPFFDTRNAQEQYPQNGDENGAY |
| | | NIARKGIIILQKITAWGRSQDTQRRYPDTFVSQDEWDTFLTQHTT |
| 7 | 291253 | MIKSYDDFTKLYSLQKTLRFELKPIGKTLEHIKKSEIIESDETLEEQYAIVKNIIDRFHRKHIDEALSLVDFTKHL |
| | | DTFKTIQELYLKRGKTDREKKELEELSADLRKLIVSYLKGNVKQKTQHNLNPIKERFEILFGKELFTNEEFFTL |
| | | AENKEEKKAIQAFKGFTTYFKGFQENRKNMYSEEDKSTAIAYRIINENMPLFIENIARFQKVLDVIEKTKLTEL |
| | | KQNLKTELKGHSVSDIFRIEYFNNVLTQEGISRYNTIIGGKTTTEGVKIQGLNEIINLHNQQSKDVKLPILKPLH |
| | | KQILSEEYSTSFKIKAFENDNEVLKAIDTFWNEHIEKSIHPVTGKRFNILLKIENLCKKLEKYKDKEIEKLFIERK |
| | | NLSTVSHQVYGQWNIIRDALRMHLEMNNKNIKEKDIDKYLDNDAFAWKEIKDSIKIYKEHVEDAKELDENGI |
| | | VKYFSSMSINEEDDEKEYSISLIKNINEKYNNVKSILEEDRTGKSDLHQDKEKVAIIKEFLDSLKQLQWFLKLL |
| | | YVTVPLDEKDYEFYNELEVYYEALLPLNSLYNKVRNYMTRKPYSVEKFKLNFYSPTLLDGWDKNKETANLS |
| | | IILKKNGKYYLGIMNKENNTIFENFPKSKSNDYYEKMIYKLLPGPNKMLPKVFFSKKGLEYYKPSKEILRIYEK |
| | | GEFKKDKSGNFKKESLHTLIDFYKEAIAKNEDWKIFKFKFKNTREYEDISQFYRDVEEQGYLIIFEKVDANYV |
| | | DKLVEEGELYLFQIYNKDFSENKKSKGNPNLHTIYWESLFDNQNLKDVVYKLNGEAEVFYRKKSIDYPEEIY |
| | | NNGHHKEELNGKFNYPIIKDRRYTQDKFLFHVPITMNFISKEEKRVNQLACEYLSTTKEDVHIIGIDRGERHLL |
| | | YLSLIDKEGNIKKQLSLNTIKNENYDKEIDYRVKLDEKEKKRDEARKNWDVIENIKELKEGYMSQVIHIIAKM |
| | | MAEEKAILIMEDLNIGFKRGRFKVEKQVYQKFEKMMIDKLNYLVFKNKEPLEPGGSLNAYQLTSKFDSFKKL |
| | | GKQSGFIFYVPSAYTSKIDPTTGFYNFIRVDVPNLEKGKEFFSKFEKIIYNTKEDYFEFHCKYGKFVPEPKNKD |
| | | NDRKTKESLTYYNAIKDTVWVVCSTHHERYKIVRNKAGYYESQPVDVTKNLRDIFSEANINYSDGKDIKPIII |
| | | ESNNAKLLKSIAEQLKLILAMRYNNGKHDDDEKDYILSPVKNKQGKFFCSLDGDQSLPINADANGAYNIALK |
| | | GLLLIEKIKKQQGKAKDLYISNLEWFMFMMSR |
| 8 | 265918 | MKYTDFTGIYSVSKTLRFELIPQGSTVENMKREGILNNDMHRADSYKEMKKLIDEYHKAFIERCLSDFSLKYD |
| | | DTGKHDSLEEYFFYYEQKRNDKTKKIFEDIQVALRKQISKRFTGDTAFKRLFKKELIKEDLPSFVKNDPVKTE |
| | | LIKEFSDFTTYFQEFHKNRKNMYTSDAKSTAIAYRIINENLPKFIDNINAFDIVAKVPEMQEHFKTIADELRSHL |
| | | QVGNDIDKMFNLQFFNKVLTQSQLDVYNAVIGGKSEGNKKIQGINEYINLYNQQHKKARLPMLKLLYKQILS |
| | | DRVAISWLQDEFDNDQDMLDTIEAFYNKLNSNETGVLGEGKLKQILMGLDGYNLDGVFLRNDLQLSEVSQR |
| | | LCGGWNIIKDAMISDLKRSVQKKKKETDADFEERVSKLFSAQNSFSIAYINQCLGQAGIRCKIQDYFACLGAK |
| | | EGENEAETTPDIFDQIAEAYHGAAPILNARPSSHNLAQDIEKVKAIKALLDALKRLQRFVKPLLGRGDEGDKD |
| | | NFFYGDFMPIWEVLDQLTPLYNKVRNRMTRKPYSQEKIKLNFENSTLLNGWDLNKEHDNTSVILRREGLYYL |
| | | GIMNKNYNKIFDANNVETIGDCYEKMIYKLLPGPNKMLPKVFFSKSREQEFSPSKKILEIWESKSFKKGDNFN |
| | | LDDCHALIDFYKDSIAKHPDWNKFNFKFSDTQSYTNISDFYRDVSQQGYSLSFTKVSVDYVNRMVDEGKLYL |
| | | FQIYNKDFSPQSKGTPNMHTLYWRMLFDERNLHNVIYKLNGEAEVFYRKASLRCDRPTHPAHQPITCKNEND |
| | | SKRVCVFDYDIIKNRRYTVDKFMFHVPITINYKCTGSDNINQQVCDYLRSAGDDTHIIGIDRGERNLLYLVIID |
| | | QHGTIKEQFSLNEIVNEYKGNTYCTNYHTLLEEKEAGNKKARQDWQTIESIKELKEGYLSQVIHKISMLMQR |
| | | YHAIVVLEDLNGSFMRSRQKVEKQVYQKFEHMLINKLNYLVNKQYDATEPGGLLHALQLTSRMDSFKKLG |
| | | KQSGFLFYIPAWNTSKIDPVTGFVNLFDTRYCNEAKAKEFFEKFDDISYNDERDWFEFSFDYRHFTNKPTGTR |
| | | TQWTLCTQGTRVRTFRNPEKSNHWDNEEFDLTQAFKDLFNKYGIDIASGLKARIVNGQLTKETSAVKDFYES |
| | | LLKLLKLTLQMRNSVTGTDIDYLVSPVADKDGIFFDSRTCGSLLPANADANGAFNIARKGLMLLRQIQQSSID |
| | | AEKIQLAPIKNEDWLEFAQEKPYL |
| 9 | 1895869 | MLKEKQFKTFGDFTNLYELSKTLRFELRPTPETKDLLDKNKIIQTDKKIAENYQEIKKYFDKLHKKFIKEALSN |
| | | TQIDFSDFCKLWEQNSKDSGKIKDLSRKLRKSIKQAFDKKGADWHKRYLEKGIKLKKKNLDILFEERVLDILK |
| | | EEFKDDVDVKLFESFKGFSTYFTNFHESRKNFYKDDGTASAIATRIIDENLKRFCDNIKVKKHSKKLISELNER |
| | | EAKIFEADFYNRCLLQQGIDDYNQVIGDINKKINNLRQNKIENTPTLKILYKQILGDVRRQETEQDAFIEIKNNE |
| | | EVFDFLQDFIKHSDENNKYFKNLFYKFIEGKHSLDKIFLAKRFVNTISGKWFASWEVFGAELIKKFGNKKDLP |
| | | DFIPFAAVKDVLQNCNIPANELFKEKIKNDEDKNIYDIFINLWKEEFDSNLKKVEESKKEVENMIAEDKVYSN |
| | | KKEKRKNDNGEEIEIEIQKEKIKNYADAAMNIFRMMKYFLLEKNGKTVEGMGEDNNFYNELNIVFKGGEIDG |
| | | KVYEGVKTYLYYNEFRNYLTKKPFNEEKTKLNFDCGQILSGWDKNKESEKLGVILRKDNKYYLAIINKKHN |
| | | KIFDVKKNSYAYIVGDNFYEKMEYKLFPDAKRMIPKIAFAKNNKEKFGWTDEIQKIKNEYAEFQEGKKNDK |
| | | NLWKDKFNKNKMEKLITYYQNCLEKGGYKDIYNFRWKSPDKYSGIGEFNDEIDRQSYCLKFVKVDFNYVFE |
| | | KVKSGELYLFQIYNKDFSDKADRAQKENIHTEYFKLLFDQRNLDNVVLKLSGGAEIFYRPKTEGLPKKKDNK |
| | | GNEVVRHRRYADDKYFLHLPIQLNFGRGNLSGGEFNSKINQYLSEQREIKIIGIDRGEKHLAYYSVINQDGKIE |
| | | EIESLNTVNGIDYRKKLDELEKKREQERKSWQSISKIKDLKKGYISHVIKKICDLAIEHNAIIVFEDLSGGFKNS |
| | | RKKIEKQIYQNLELALATKLNYLTFKDKNFGESGHYLNAYQLAPKIDNYQDIKMQTGIVFYTPAGYTSSTCPQ |
| | | CGFRKTLKFDYTATISKAEDLIRGSKLNIVFEKEKNRFKINYLFNPIEKKKKKIKENELFADAGAKNEFTIYSD |
| | | VKRIKWHNTGTKRLEEAEGERLLENKNSRGRDKEYDINKCLTRLFRENKIDVNGDIIGQITKIKSLKLYQDLF |
| | | YYLFLATLIRNNVSGSDIDYIQCPSCHFHSDGGFQKQKFNGDANGAYNIARKGILILKKIKQFAAQDKDMKNF |
| | | GWKHLTVDINEWDKFTQK |
| 10 | 1895866 | MNKNFSNFTELYTLSKTLRFELKPVAQTKENIKKGKFLESDKKKADDYKDVKKIIDNYHKFFIDDVLKNASF |
| | | DWTVLEKEMSDFNKSKADDSKVEAEQKKLRDQIAKKLTSDKRFKALTASTPSDLFNKDKDFIDWFTQNSTK |
| | | DINKEALETFKRFSSYFKGFQENRKNVYSAEPIPTAVPYRLVNDNFPKFLQNIALFKIIQEKCPQVISDVEKELA |
| | | SYLGKEKLADIFTVQAFNKYLCQGGKENQRGIDFYNNILGGIAEKEGGINLRGINQFLNLYWQQHQDFAKQN |
| | | KRIKMIPLFKQILSDRSSLSFKIESINTDQELLTSITEYADKLETKSNDEKKSVLDICSDLFASIKAQNLQEIYVN |
| | | RKDINSISRILTGDWSWLQSRMNVYADEVFTTKAEKTRWQKSIDGDEGENKSKGVFSLAELNSVLEYSSENV |
| | | SETDVRITDFFDHRNRFYYEKESGLFKQGDELVALSIRESCEDILAKRKAMDEAFANVSENNSLRDNSEDVAK |
| | | IKIYLDCVQELLHRIKPLKVNGLGDPAFYAVFDTVYNSLSEVISLYNKTRNYITKKAANPEKYKLNFDNPTLA |
| | | DGWDLNKEQANTSVLMRKDGMYYLGIMNPKDKPKFAEKYECGNEACYEKMIYKQFDATKQIPKCSTQVKE |
| | | VKKHFQSGATDSIILNDKSKFKLDLVITKEIWFLNNHVWNGEKFVPKRESNETRPKKFQIGYYKQTGDLGGY |
| | | KEALNIWISFCKTFLQSYISSSIYDYDFKESSNYDSLDEFYNYLNATCYKLSFINIPEATISQMVSEGKLYLFQIY |
| | | NKDFAPGASGMPNMHTLYWKNLFSEENLKNVVLKLNGEAELFYRPAGIKEPVIHAKGSYLVNRITKDGEPIP |
| | | EKIHDEIYRNANGKLESLSKEATEYKASHKVVIKEAKHDIIKDRHYTEPKFLFHVPLTINFKASGNSYINENVR |
| | | RFLKNNPDVNVIGLDRGERHLIYLSLINQKGEIIKQFTFNEVERNKNGQVIKVNYHEKLDQREKVRGAARKS |
| | | WQAIGKIAELKEGYLSAVIHQLTKLMVEYNAIVVMEDLNFGFKRGRFHVEKQVYQKFEHMLIDKLNYLVFK |
| | | DRGLTEAGGVLNGYQLAGQFESFQKLGKQSGMLFYVPAGYTSKIDPKTGFASMSNFKDLTNVHKKRAFFSK |
| | | FDDIHFDDATGSFVFTFDYKNFDGKAKEEMKRTKWSVYSKDKRIVYLSKTKSYEDVQPTEKIKASLESVGIE |
| | | YMSGNNLIDSIMVIGAELKDGAKPSKEIADFWDRLLYNFKLILQMRNSNAKTGEDYIISPVMADDGTFFDSRE |
| | | EFKKGENAKMPVDADANGAYHIALKGLSLLKRFDAASENELKKFDMKISNVDWFKFAQEKSYAE |
| 11 | 1895860 | MNFDEFTNLYSISKTLRFELKPVGKTLDWITKNKLVEEDEKRAEDYKTVKKIIDRYHKEFIEEAFESALNANSF |
| | | QRDMKDLIESYSELYYKEDKSDKDKKELENISSQMRQEIVSCFEGNYSDEVKKKFSNLFKKELIKQDLLNFCQ |
| | | PSEKEVIEKFADFTTYFGGFHENRKNMYSSEEKSTAISHRIVHENLPRFLDNLKVENSLKEEYQDFPWNQLEF |
| | | NLKNIDPSLKLEDYFSKKGFILTFTQKGIDRYNLILGGKSLDSGQKVQGLNELINLYRQKKCLDRRKIPNLKEL |
| | | YKQILSDRVKFSFIPEKFLSDNSLLEAIRNFHEEVINAKIEIDSKQVNFLEALKNTIQGIKAHKLEKIYISNDLGL |
| | | TNISNFLYGEWSKIKSALFYYYDEKIADKGKGDKQSKKYQEGREKWIKKDYYSVQKLNEAITLYSKYSEDGF |
| | | EATPLEEYFSKFKTRDENRAELDILSNIEQSYSELKPLLEKEYLKNKNLKSDKYNIEKIKNYMDSIKILQNFLKP |
| | | LTPKKILEEKDLGFYNEFETYVESLQSFNRLYNKVRNYLTEKEYSVEKFKLNFENSTLLNGWDENKESANLGI |
| | | ILRDEDKYYLGIMNKNYKKIFSNIPKLKSSEAYYEKMVYKLLPGANKMLPKVFFAKSNIGFFQPSQKILDIKNS |
| | | ESFKKGDNFNLKDCHELIDFYKESISKHEDWSKFNFHFSETSSYQDISEFFREVEAQGYKITFNQVSKDYIHSL |
| | | VEYGKLYLFQIYSKDFSEKKKSNGKPNLHTIYFKALFDEENLKDVVVKLNGEAEVFYRKKSINYDPEIREKGH |
| | | HYEELKDKFTYPIIKDKRYAEDKFHFHFPITLNFKPKERSNFNLEVRKFLKASKDVNIIGIDRGERHLLYLVMI |
| | | NQEGKILKQLSLNTIQNEKQAVNYKEILQKREIARNQARKSWRTVENIKELKEGYLSQVVHKISKMMVEHKA |
| | | IVVLEDLNLGFKRGRQKVERQVYQKFEKMLIDKLNFLVLKDRKNNEPGGILKAYQLTDKFVSFENIGKQTGF |
| | | LFYVPAWNTSKIDPKTGFVNFLHLKYENVQQAKDLIGKFDSIRYNKQKDWFEFQVTTDKFFTKENLPESRTW |
| | | TICSTPTTRYYTEKTTNSSIVTNEINVNEKLKELLSEFHFEDGKELKEKILQKDTKDFFSKLIFYLRVLTSLRQN |
| | | NGKIGDQEKDFILSPVADSKGNFFNSLEAKDDEPNDADANGAYHIALKGLMNLQVLKQTQEENLDKPNWKI |
| | | KNTDWLHFVWNRQN |
| 12 | 1895861 | MNFDKFTNLYSISKTLRFELKPVGKTLDWITKNKLVEEDEKRAEDYKTVKKIIDRYHKEFIEEAFESALNANS |
| | | FQRDMKDLIESYSELYYKEDKSDKDKKELENISSQMRQEIVSCFEGNYSDEVKKKFSNLFKKELIKQDLLNFC |
| | | QPSEKEVIEKFADFTTYFGGFHENRKNMYSSEEKSTAISHRIVHENLPRFLDNLKVFNSLKEEYQDFPWNQLE |
| | | FNLKNIDPSLKLEDYFSKKGFILTFTQKGIDRYNLILGGKSLDSGQKVQGLNELINLYRQKKCLDRRKIPNLKE |
| | | LYKQILSDRVKFSFIPEKFLSDNSLLEAIRNFHEEVINAKIEIDSKQVNFLEALKNTIQGIKAHKLEKIYISNDLG |
| | | LTNISNFLYGEWSKIKSALFYYYDEKIADKGKGDKQSKKYQEGREKWIKKDYYSVQKLNEAITLYSKYSEDG |
| | | FEATPLEEYFSKFKTRDENRAELDILSNIEQSYSELKPLLEKEYLKNKNLKSDEYNIEKIKNYMDSIKILQNFLK |
| | | PLTPKKILEEKDLGFYNEFETYVESLQSFNGLYNKVRNYLTEKEYSVEKFKLNFENSTLLNGWDENKESANL |
| | | GIILRDEDKYYLGIMNKNYKKIFSNIPKLKSSEAYYEKMVYKLLPGANKMLPKVFFAKSNIDFFQPSQKILDIK |
| | | NSESFKKGDNFNLKDCHELIDFYKESISKHEDWSKFNFHFSETSSYQDISEFFREVEAQGYKITFNQVSKDYIH |
| | | SLVEYGKLYLFQIYSKDFSEKKKSNGKPNLHTIYFKALFDEENLKDVVVKLNGEAEVFYRKKSINYDPEIREK |
| | | GHHYEKLKDKFTYPIIKDKRYAEDKFHFHFPITLNFKPKERSNFNLEVRKFLKASKDVNIIGIDRGERHLLYLV |
| | | MINQEGKILKQLSLNTIQNEKQAVNYKEILQKREIARNQARKSWGTVENIKELKEGYLSQVVHKISKMMVEH |
| | | KAIVVLEDLNLGFKRGRQKVERQVYQKFEKMLIDKLNFLVLKDRKNNEPGGILKAYQLTDKFVSFENIGKQT |
| | | GFLFYVPAWNTSKIDPKTGFVNFLHLKYENVQQAKDLIGKFDSIRYNKQKDWFEFQVTTDKFFTKENLPESR |
| | | TWTICSTPTTRYYTEKTTNSSIVTNEINVNEKLKELLSEFHFEDGKELKEKILQKDTKDFFSKLIFYLRVLTSLR |
| | | QNNGKIGDQEKDFILSPVADSKGNFFNSLEAKDDEPNDADANGAYHIALKGLMNLQVLKQTQEENLDKPNW |
| | | KIKNTDWLHFVWNRQN |
| 13 | 1895864 | MNRTLDDFTNLYSLSKTLRFELKPIAQTEENIRKGKFLESDKRKSDDYKDVKTIIDNYHKYFIDDVLKNACLD |
| | | WSQLEKEIIEYNKNKSDDSALAAEQKKLREEINKLFSTDKRFKALTAATPNDLFKSILPEWFGNNELPGVNKQ |
| | | AVETFQKFSSYFTGFQENRQNVYKSEPIPTAVPYRLVNDNFPKFLQNIVVFKIIQEKCPQVISDVENELASYLG |
| | | NEKLKDIFAVSSFNKYLAQKGAEYQKGIEFYNQILGGIVEQESGQNLRGINQFLNLYWQQHPDFAKNNRRIK |
| | | MVPLFKQILSDRSSLSFKIASIESDEELKAALLECADRLEESTDDKNSVFEECLDLFGTIKDQNLNEIYINRKDIN |
| | | SVSRILTGDWSWLQSRMNVYADEKFTTKAEKARWQKTIDGDEGENKSKGFYSLAELNNVLEYSSENVSETEI |
| | | RITDYFEHRNRFYIEKETDNFIQGDEFVAFSIKESCSDILSKRKNMNSALENHSQEHPLRDDSGTVEIIKAYLDS |
| | | VQDLLHRIKPLKVNGVGDASFYSAFDDIYASLSEVVSVYNKTRNYITKKAASPEKYKLNFDNPTLADGWDL |
| | | NKEQANTSILLRKDGMYYLGIMNPKNKPKFLEKYENTNNEKCYEKMIYKLLPGPNKMLPKVFFSTKGKEVF |
| | | NPPEYILQGYEQGKHKKGESFDISFCHELIDWFKSAINQHEDWKKFDFHFSDTKTYRDISDFYREVTEQGYKL |
| | | TFINIPESEIQAKVSEGKLYLFQIYNKDFAKGATGMPNMHTLYWKNLFSEENLKNVVLKLNGEAELFYRPAGI |
| | | NEPIVHKAGSYLVNKVSEDGETIPDSIYQEIYKNANGMLNSLSEVAERYKAEHNVIIKKAAHDIIKDKHYTEA |
| | | KFLFHVPLTTNFMAPSMKPAVCTAIQNENVRKFLKNNPDVNIIGLDRGERHLIYLSLINQKGEIIKQFTFNEVE |
| | | RKKNNQIVKVNYHDKLSQREKERDAARKSWQTIGKIAELKEGYLSAVIHQLTKLMVEYNAIVVMEDLNFGF |
| | | KRGRFHVEKQVYQKFEHMLIDKLNYLVFKDRGLTEAGGVLNGYQLAGQFESFQKLGKQSGMLFYVPAGYT |
| | | SKIDPKTGFASMLNFKDLTNVHKKRAFFSKFEDIHFDSVTNSFMFTFDYKNFDGKAKEEMQQTKWSVYSKD |
| | | KRIVYFPKSKSYENVDVTSKLKALFDNAGIKYQSGNNILDDVMAIGADLKDGTKPSKEVAEFWDGLLYNFK |
| | | LVPQMRNSNARTGEDYIISPVMADDGTFFDSREELKKCGNAKLPVDADANGAYHIALKGLSLLKRFDAADE |
| | | DDLKNLKINISNSDWFKFAQEKNYAK |
| 14 | 266750 | MKKFINLYPVQKTLRFELRPVGETLKNIEEKGILTADEHRSYAYGKVKKFIDEYHKAYISKSLSGLVLDASAL |
| | | NDVLLFSGRTDETLRNKASKAMDRLRKQIADSFDTKSLFSDKVIKEDLPAFLDDDDKTQLVAEFGNFTTYFS |
| | | GFNENRKNMYTSEPKATGIAYRLINENLPKFISNMKSFGSIREPLADQIKELKSYFEPMLEGIQVEDFFSDITYY |
| | | STLLTQEMITIYNTLIGGKSEGATKTKGLNEYVNLYNQTHKDKLPKFVPLYKQILSDRDSLSWLPETFKSDGE |
| | | LLESVETFHRLFESRIVNGNDGTISLKELLVSMEGYDLNGIYLSTGAGLNSIMKSHTNDWDALNKAIEEDFNR |
| | | KYPCTKDKDMPKFLEKRARYVKSFDSLSIADINGYIGEGKALQHYFMTLGASSDNERTLFERINESYSAIRELL |
| | | NTEYNKNLIQQDEDIEKLKTYLDSIKELQWFIKPLNGTGTESGRDGRFYGDFDKLYGILDKVTPLYNMVRNY |
| | | VTRKPYSEEKIKLNFDNSTLLAGWDTNKERDNSGIILRKGGRYYLAIMDKMFNKSFDLDSLPCKGECYEKMD |
| | | YKLLPGPNKMLPKVFFSESRIDEFAPSKSLLEHYSKGTHKKGVDFNLNDCHELIDFFKASIEKHKDWKQFNFH |
| | | FSDTVTYSDISGFYKEVEQQGYKITFRDVSCSYIDQLVNEGKLYLFQIYNKDFSEYSKGTPNMHTLYWKALFS |
| | | EENLKDVVYKLNGDAEVFFRRKSIQYDSEKMKKGFHHETLKDKFSYPIIKDRRFTMDKFQLHVPITMNFKSF |
| | | GINNINMMVREYIRDGEIEHILGIDRGERNLLYISLIDLNGNIVKQLSLNEIINKYNNYEYKTDYHSLLEKKEVN |
| | | RLDARKNWKTIENIKEIKEGYLSQVIHIISQMIVEYKAIVVLEDLNSGFLRGRQKVEKSVYQQFEHMLINKLN |
| | | YLVDKKSPNLEDGGLMRAYQFTSKFESFQKLGRQSGFLFYIPAWNTSKIDPVTGFTNFISTRYESIDSARELLG |
| | | KFEDIRYNAENDWFEFVIGDYSSFNPKAVGRKEWTLCTYGTRIRSFKNEKKNNEWDCEEIHLTEEFKKLFGS |
| | | HGIDYADNLMGSILIQSNKDFFSGILSLLGLMLQLRNSRTGTEEDYIISPVAGQDGRFFDSREYSSADAPLPTD |
| | | ADANGAYNIARKGLWAIKQISIADDVSKVNLAISNESWLKFVQG |
| 15 | 275402 | MFDQFTNLYELSKTLRFELKPVGKTRENMNEHLKYDEVLKTFMADQKIEDAYQTLKPVLDALHERFINDSLE |
| | | LDEVKNIDFSEYLEKYRDKDLKNKDFEALERKLRVKFKKLYEKVGENWIAKAGFNDKGKPVFKEKSFKILTE |
| | | KGVLDFIKKNADNFTFVSKEQLEGAIKVFENFFTYFTGFNRNRENYYETGKEAYTAVATRIIHDNLPKFCDNL |
| | | IVFESRSKDYLGAYELLKGRGLSLVTKEGRPLMPIVDNIFRIDYFRKCLSQAQIEEYNKQIGNANFVVNLYNQ |
| | | VFSGNSDFKKLLSFKLLYKQIGCGKRRSFFVELTHDTIAEADKARGEGKKAFSVEELVLLAKQAGEIYFQGKS |
| | | DDGIINTVPELLDYLEKKSDYRGIYWSKTAINTVSGKYFANWFDLKNKFKEAKVFEKNKKGSEEEVKIPDVV |
| | | ELEDLFSVLNEWSDWKTTLFKSSIMQDAKKRELIEKSPNASTALLSLLFADIKDHAECFIDEANEVSQVKDFK |
| | | KEENKILIKLWMDHALVVCKMLKYFLVREDKVKKVKGALMDATIVEALNNILSGQVEVDKNGKKIAAEVD |
| | | WFEWYNYFRNYLTKKPQDDAKKNKLKLNFECASLLGGWSDGQEKEKGAVLLKKDNFYYLGILKKKNLFD |
| | | TEKENNPVYQNPTFSAGRLILANLKFSTLAGKGFLYEFGKAYGDMGAEDPDKAIKCLKKIIKDRYEEKYPLL |
| | | ARIAATHYSDKKVFDKDIQETLKECYVCRFEPVSWIKVEEYVDDGELYLFQIYSKDFATNNTGKNDLQTLYW |
| | | KAIFDEYSSFQLNGRGEIFYRKQAIKEKIVKVGYEGKPWVIADKRFTLEEGKFAFHCPIKINYKAKNYSQPKFA |
| | | FSEINKIINDGFGKFSNIYFLGIDRGEKHLAYYCLVDQNSKIIEQGSLNMPFVDASGNPRMVKTERKFIQEDGK |
| | | EQLEIVECKDYNELLEARAGNRDYARKNWQVIGTIKELKEGYISQVVHKIAGLCANKEHPVFIVLEDLNTGF |
| | | KRGRQKIEKSVYQKLELALAKKLNFLVDKNAKLGEVGSVTKALQLTPPVNHYADIKNKKQVGVMLYTRAN |
| | | YTSQTDPKTGWRKSVYLKPGSEENIKNQILEQFSDILFDGKDFVFVYADANTGTEWKLYSGINGISLDRFTVK |
| | | KGVKNEWCIEKQDIVEILEGVFSKFEKNRSFLSQIKDEGRELTKISDHTAWESLRFAIELIQKIRNTGITSRDND |
| | | FLLSPVRDSAGNHFDSRVYLDKEQRGEKIDLPSCGDANGAYNIARKGIIMWEHINRGYRPYVSDDEWDAWL |
| | | AGVTNWEKWREGNEKLLKYNK |
| 16 | 287482 | MKKFIAKYNISKTLRFELKPIGKTLENIQKNGLISKDEMLAENYKEMKKTIDEFHKEFIHKTLSNASLDKENLK |
| | | EIAELYTAPAEKRKEDNYKNQLEAYQKALRKEIVDRFKNDELFKNLFSEKLIKNHLQGFAQRKGLRFYSEFN |
| | | NFTTYFVGFHQNRKNIYTDQAHSTGIAYRLIHENLPKFIHNISVFEKIQEKKIPLTILENQFMSKLGNDTLADVF |
| | | RLEYYNCLLTQEGIDFINQLIGGYVNENQEKIQGLNEYINLYNQQQKDKKDRLPKMKMLYKQILSHRESISFL |
| | | PQSFKESQEVLDAVRDYYLSNLLEYRAEDKEEPENILEKIQERLSHIADYDLSKIYIRNDKAITDISQTIFGHWG |
| | | LIHSALEFDFRQRNVGKKTTQKQEENVEKYLKSDYYSIAEIENALISYKESTQNEKLVEVFNKTSHPIADYFKT |
| | | YFKAKEKSKEGKEYTLIANIQAKYTCVEGLLNTDYPKDKKLHTDKDNLRNLKAFLDAILEFLHFVKPLILRK |
| | | NDFLEKDENFYSVLETYYTQLQLLVPLYNKVRNFATQKPYSTEKIKLNFENSTLLNGWDKNKESDNTCVLFR |
| | | KGKDFYLGIMNKKYNKIFENVPKPKSDTTFLKMEYKLLPGANKMLPKVFFSKKNIAYYAPDSEILRIRNHSTH |
| | | TKDGEPQKGYEKQAFKLEDCHKMIDFFKSSIEKHPEWKNFNFIFSDTQHYQSIDEFYKEVEELGYSIGFVEVD |
| | | EEYINQCVNEGKLYLFQIYNKDFSPNSKGKPNLHTLYWKAIFDPENLKDVIYKLNGEAEVFYRKKSISADNIIV |
| | | HKAQKPVENKNPLNTKKYSTFEYDIIKDRHYTKDKFLFHVPITLNFKSKKGKENINQEVLDYLKENPDVNIIGI |
| | | DRGERHLLYLTIINSKGEILHQESLNKVKNEVYGIEIETDYHLLLNKKEEERNKARKEWGTIENIKELKEGYIS |
| | | TITHKIAKLMVQYNAIVVMEDLNSGFKRGRIKVEKQVYQKFEKMLIEKLNYLVFKDVPNYEPGGLYNALQL |
| | | TNKFESFEKLGKQSGFLFYIPAWNTSKIDPTTGFVNLFGGIKYESIHQAVNFFEKFQSIRYNTKEAYFEFAFDY |
| | | NDFTTKAEGTKTQWTVCTHGERIINFRNTEKNSEWDSKTVQPTQEIKNLLNEYHIAYEQGQDIKPAITAQNDK |
| | | KFFEELLKQFKNTLQMRNSVINSDVDYLISPVMNAKGEFFDTRKTDSTLPQNADANGAYHIAKKGLLYVQQI |
| | | IDGVKKTDLTNKKWLKFVQNNL |
| 17 | 299207 | MGFKYMQAYRCLKKFVNQYTLQKTLRFELVPNPITKKILEIESAKKSFLNDRERNKAREELKEMLKELDNEFI |
| | | QEALRDFQFSKQDLQELWEKYKKDKKSIQDKDLKKLESKIEKRFSCFSSSKYGKIKFTKQNILSEKVIKILKEK |
| | | YKDNQQKQKIIDLFDRFTTYITDLKQNRENYYKAEGKTSQFATRAVRDNLIRFFDNKFIFENKYSQNFGFSEE |
| | | EKKIFLLSYYNGCLLQKGIDRYNELVGNLNSKINQYILKHKNNHDFKKSDYPFFKKLDKQIVSSKDVRKSIEIN |
| | | DDQELIECLDCFYRFAQGIIPAIKKLWQKFFEPERELGKIYLPRNKANRLSYYFFGDWSLLSNVYGRGEKKQE |
| | | FVSFRKIKEYLQDFDNLEFRKLTANQDIIKKEKDNFANFIDAWKYEGEVLFSDKKEKSIFIFPEGKKEFKSLVD |
| | | LEKGFLEKVNWFRKKIENKEKISDKEESKFKMAVKDYLDRVLDIFRLVDNFYLKEEQRNKIPVEELDSEFYKF |
| | | FDEYQEKISDFNPYKYYDEFRNYLTKRNVVADKIRIAFDEGQLLGGWDLNKEKKKLGLIFRKNGNYYLGIIN |
| | | KKKDKNIFDKNKHPEAFVESSKWEKMEYKLLGDLKRQIPRIAFSEKNRDIFGYNSEIQKIKEEYDQLQNSKKE |
| | | NRDNWHKKFNKRKLIKLISYYQKVLEFHPERYKENYKLSWKKPKEYNSLGEFYDDIDKQMYRIKFVNVSDD |
| | | YLEEKVVKGELYLFQIFNKDFSSYKESNSKENLHTIYFRELFSEENLKNPIFKLSGGAQIFFRDKLEEYQIKTKK |
| | | DKKGKTVIDRPRYAKDKILFHLPIELNYKAGKANKFNQKINKFLASNPDIKILGIDRGEKHLLYYSLIDTNGKI |
| | | LKSGSLNKIKIGDKEIDFHQKLRERAKEREEARKSWQSITQIKNLKKGYISLVIKKIADLAINENAIIVLEDLNTP |
| | | FKQKRGGIFEASIYQQFEKALIEKLNFYVDKEKKNHRRAIQLTAPFTTFERIDKQSGIIFYVPANYTSVTCPECG |
| | | WRQRLYLKYKNINQAVKDFERIKVIYEPTKDRFVFEYGFEVKDNKKKNSFKGIDKIYSDVERLWWNPKYNE |
| | | NGKYGRIEKLGLGEITKELKNVFKEMEIDYHQEGNLSEQLTSKTDKKYSKHWRELYWLFRMIAQIRNLDREN |
| | | DEENKDFILCPACHFDSRDKNTLIPNGDANGGYNIARKGIILLQRITANPQKPILTISNRDWDEFVRR |
| 18 | 264288 | MIESKLSKYLNLYSISKTLRFELKPVGDTLKNFKSDNILEKDKEKALKSKEVKKILDEYYKEKIDEILNNNLFE |
| | | KSILDEYIKNQNIKENIKIIKDIMKSTIVSRFESKNILENGKEVTTLKLLTSELVFKSLDIMLEKANRIEDLKKIKL |
| | | FDKYTTYYTDFFKNREFIFKGKDQGSIAYRLIDENLDIFMDNLNNYNQNVPEEIKNKFNNKSIDFLVDIESFNS |
| | | VLTQKGIELYNLLISGQTLEKENKVQGLNELINLHNQLNKTKIPLMKILNKQILSDKQTFSFVLDIIEDDETVFA |
| | | LIDEYIKNTCFINLFENKDINYDEVYITNNQNITLLSKKLLSNWNEIKNLLYSWYEKNIDHKTENKNFLEKRTR |
| | | FFKNTDHFSIKFLSNLTDNRVNSSFFITESNDYILKIKSSIDEYRKIIRTNIKNLKKSEEAVLKIKALLDSIKEYQE |
| | | FLKQFLMSDIRVQTLKSLEEGVNSLSSKDSLFYNDFDNSYNNIFSIVAIYDKIRNYLTKKPFKTDKIKINFNCST |
| | | LLNGWDLNKEKANLGVIFKKYDINIKKDIYFLGIIKDKKIFDKLKETDEQNCYEKMEYKLFPGANKMLPKIFIS |
| | | AKAYRANLSEEFLKNYSEKKHTKSNLDEKFLAEYILYMQKSLNDYYKDEFNFKFNKAESYKQIDEFYRDVEE |
| | | QGYKLKFKKISESYINKCILENKLYLFQIYCKDFSNYSHGLPNTQTIYLQSLFSNDNLNNPYMKLLGNAEIFYR |
| | | EKSLEIIETHKANEQLQNKNVKNLKKKSQFNYSLIKDKRYTEDKFLFHFPITINYKSGNIRNINQLINANINDFE |
| | | HVIGIDRGERNLVYIVVSDLKGNIKDQISLNEIVNSHNENEYRTDYNDLLEKKAKGRDEEKKNWSEIENIKEL |
| | | KEGYISQVVSKIKDLALKYNALIVLENLNFGFKNSRIKVEKQVYQRLETQLINKLNYVIDKKDSRTYLNGIQL |
| | | TNPINNLSEIGSQSGIVIYIPAWNTSKIDPTTGFVNLIYGLKYKNIADSKEFISKIKDIRFNGKYYEFDIDFGEFNN |
| | | KLKETKLNWTICTYGTRVETRRDEKNNNEWVSKEVDLTKRFEELLKKYNIHSDFKNQILLQNSSDFFEEFIHIF |
| | | KLILQIRNSIVGTKIDYMISPVKNPSGKFFDTREGIKTLPLDADANGAYNIARKGIMIIDRIKNTTIGEKIDYKISN |
| | | EEYLLYLQNQ |
| 19 | 1895875 | CTSRRRIAMKSIFDNFTGLYSLSKTLRFELKPEVETKKRFDNWIKEVESNNESTAENLIAKDYKIFNAYKTLKP |
| | | ILDSIHEQYITIALQSSVAKKIDFTCFLPEYSDKTKKLSKIEEKLRKEIGETFKVSEEFFFEKIKAVEPKVKSKSK |
| | | DSILKDQYVLTYIEKNIDLFTSPSLSEDELKQHLKAFKGFFTYFSGYNINRENYYDCSSEKNTAIATRIVNDNLP |
| | | IFCDNKIRFEKNKKCYLEAYQVLKNAGIETKIKLSDDTFSEAKPISEDIFEMSYYNNCLSQSEIETYNKMIGNA |
| | | NLIINLYNQHAGASDNNFKRLPVFEQLHKQIGCGKKKSLFFSLKKDKETELSLEERQSEEILSVEKLLNVTAEA |
| | | GNAIFNGSKESNAYSINFFTDFLLEQTDWHGIYWSDTATAYISGRYLLNWHELKDQLKNYSSCASFDKKREE |
| | | QVKLNEAVELAGLFEVIDKENVENIFKTSVLEKYSDVIKTDEPVSKNLIKIICADLKCSLDYFNTNSKKILSMD |
| | | SYKTKSAYADEDDASVMQIKNWYDSIKEILRIVGLFKVKKNKMKGSLSIPEFENCVDAILSPEGIYWNKWYD |
| | | GIRNYLTKKPQDDVKENMLKLNFGSSSLLGGWSDGQEKVKISTILKFENDIFLCILKNKKFFDTGKKENPVYK |
| | | GEGGFRLILRNLKFQTLAGKGFVSAYKQGYSEMGKENPQKAMECLIAFIKENYVKKYPALKKIVEKKYTSKK |
| | | EFDAAIQDVLKECYECSFVPIDWNIIQTAEKKGDIFLFKTSSKDFKEKSSGKKDLQTIYWESVLSENSPHQLCA |
| | | GAEIFMRKPIQAKSPVVHKAGDILINKKFSDNKTVPDEFYKELMKFVSNIKQPVENEQVVKDLLFKSELAKNN |
| | | TKEYLDKVKFKIAKHYIEKDRRFYGETKYFFHCPIKLNYNQKSYSKPEYAIKEMNDLINEGLSKQEAVHFIGL |
| | | DRGEKHLVYLCELDENCNIIECRSLNVINGTDYMLKLQEKSDNRQKSRQNWQKIDNISNLKDGYTSHVVHA |
| | | VTEKAVRTPSYIVLEDLSRQMMQGRQKIEKSLYQKFEVALAKKLNFIVEKDIPQGEPGSVSLPLQLTPPLNTF |
| | | KDIENKKQFGIMMYTRANYTSVTDPVTGWRKTIYIKNGSEEDIKNQILEKFSDFGFDGKDYYFEYKDANTEY |
| | | TWRLYSGKDGEPLPRFQNKKQLQNDKNIWVPEKIDVVEILDKLFSDFNKEKSFKEQIMNGINLKKIDGRSETA |
| | | WQSLLYAINIIQQIRNSGISEKDNNFLYSPVRNNNGIHFDTRIQDNKTLPADADANGAYNIARKGIIMYNHIQQ |
| | | WKKEGALEKDLDLFVSDEEWDLWLLNKKAWKEKLTYFASKSKKQKKQ |
| 20 | 265956 | MEKEKTKSIFDNFTRQYSLSKTLRFELKPIGKTRENMNANLEYDENLRTFLADQKIEDAYQILKPVFDKIHEEF |
| | | ITKSLESAAAKEIDFSEYFNLYKKQKNEQNKDERKKLDKLIQNEEKKLRKEFEKFYLIAGEEFKIIAGKDSNGK |
| | | DILKKNGYKVLTEAGILAYIKNKLLANPAWLDDLKIPEVKVLVENKEKNKEKVEKALEQMKRFFTYFGGFN |
| | | QNRENYYDVSANKNGEGKSTAVATRVIEENLPKFTDNLLSYEKNKDEYDKVFLKLQNDNKELRLRNPRWD |
| | | GIDKNTEFIPAYDIKNDGTSFAIGNFVNCFSQSEIDAYNAKVANVNYLVNLYYQQTKNKLPKFKILFKQIGCG |
| | | EKKDFIKLIKNNAQLKAVLEKAKLAGEKYLINDKDKDAVGAFMDYLLNLDDFKNIYWSDKAMNTISGKYFG |
| | | NWGNLKDKLLKAKVFNKDKNSGEAKIPQAVSLSDLFEVLDAERDWQGKGILFRENFKENNEEKQDIIKNSRT |
| | | PHDALLKMICKDIENLAQKFVDGADEILKIKEGDYQKDESKIAIKTWLDSALFVGQILKYWRVKARFSVDSN |
| | | FTDILDKVKVSETVQDYDVVRNYLTQKPQNKLGRLKLNFENSTLAGGWDVNKEKDNACLILQDKKGKQYL |
| | | VVTECSGAGIFEQTAQNSLYAPDASGWTKMNYKLIPGPNKMLPKVLFFSSKWIKANPTPDNIKTIYEKGTFKK |
| | | GDNFSQNDLHALIDFYKDQLKKYPLPEESWNRIFDFDFSDTKKYKSIDQFYTEVERQGYKIEFVPINKGKIDEL |
| | | VEGGRIYLFEIKSKDSNLKNGKEKTSAKDLQTIYWNEIFGDAENKPKLNGGAEIFYRSALEEADLKKKNWKN |
| | | KEIIENFRFSKEKFILHCPITLNPCLKNNRINDLVNKNFAKSKNQLFLGIDRGEKHLAYYSLVNRKGQILEQGSF |
| | | NTINGQDYWDKLDKKQGDRDEARKNWTTIGNIKDLKDGYISQVVRKIVDLAVYNEGDRKKGFRETPAFIVL |
| | | EDLNIGFKRGRQKIEKQVYQKLELALAKKLNFLVDKNAKVGEMASVDNALQFTPAVREFSDIKGKQFGIML |
| | | YTNPSFTSKTDPITGWRQSIFIKKGSVKYIQGQIMGSFSDFGFNGKDYYFGYEDVNTGKEWTLYSGKNGRELD |
| | | RYRDKFSEKEGKKHWNPEKIEIVKKLEKIFDGFDKNKSFKEQIKSGKQINKIDEHTAWESLRFTIDVIQQIRNT |
| | | GNNEKDNDFILSPVMNDNGDFFDSRDIKDNEKFPQNGDGNGAYNIACKGIIMSEHIKRGLNLFIRSEEWDAW |
| | | LAGEKIWEKYIAKNLKIRKKVS |
| 21 | 266110 | MKQFINLYPVQKTLRFELRPVGETMKNIEEKGILTADEHRAYAYGKVKKFIDEYHKAIITKNLSGLVLDSSAL |
| | | NDVLIFSGKTDVTLRNKANKAMEKLRKQIADSFDLKSLFSEKVIKIDLPAFLNDDDKNQLVAEFGNFTTYFSG |
| | | FNENRRNMYTAEPKATGIAYRLINDNLPKFIGNIKAFEAVKEPLANQIEELKSQFEPMLEGIQLEDLFSDVTDY |
| | | SVLLTQEMITLYNTLIGGKSEGSVKIKGLNEYINLYNQTHKDKLPKFVSLYKQILSDRDSLSWLPETFKSDSEL |
| | | MESIESFHRLFESRISNGDDETISLKELLVSIADYDLKGIYMSTGTGLTSVMKSHTGDWNTLNKAMEEDFDRK |
| | | YPCTKEKDMPKHLEKRAKYIKSFDSLSIADINVYIGEEKALQSYFMTLGASSENGKTLFERIEENYCVIKDLLN |
| | | TEYSKNLTQQDDDIKKIKTYLDSIKELQWFIKPLNGTGSESGRDGRFYGDFNELYGLLDKVTPLYNMVRNYV |
| | | TRKPYSEEKIKLNFDNSTLLAGWDVNKERDNSGVILRKDGLYYLAIMDKKFNKSFDMDSYPCEGECYEKMD |
| | | YKLLPGPNKMLPKVFFSKSRISEFTPSQSILDHYSRGTHKKGEDFSIDDCHKLIDFFKASIEKHEDWNKFGFHFS |
| | | DTSTYNDISGFYREVEFQGYKISFRNVSCTYISRLVSEGKLYLFKIYNKDFSTFSKGTPNMHTLYWKALFSEDN |
| | | LKDVVYKLNGDAEVFFRRKSIQYDDEKMKKGFHHEQLKDKFSYPIIKDRRYTVDKFQLHVPITMNFKSYGID |
| | | NINMMVREYIKDGEIDHIIGIDRGERNLLYISMIDLDGNIVKQLSLNEIINRHNSCEYRTDYHSLLAKKEGDRL |
| | | DARKNWKTIENIKELKEGYLSQVIHIIAQMMVEYNAIVVLEDLNSGFMRGRQKVEKSVYQQFEHMLIDKLN |
| | | YLVDKKRPVMEEGGLMRAYQLTSKFESFQKLGRQSGFLFYVPAWNTSKIDPVTGFTNFISTKYENIDSARELL |
| | | GKFKDIRYNAEKGWFEFDIDDYSSENPKAVGRQEWTLCTYGTRIRTFRNERKNNEWDSEEIHLTEEFKRLFGS |
| | | FRIDYTENLLGSILIQSSKEFFSIILSLLGLTLQLRNSKTGTDEDYIISPIAGPDGRFFDSREYDSKDSTLPSDADA |
| | | NGAYNIARKGLWTLRQISLADDLSKVNLAISNEGWLKFVQK |
| 22 | 274638 | MNKNFSNFTELYTLSKTLRFELKPVAQTKENIKKGKFLESDKKKADDYKDVKKIIDNYHKFFIDDVLKNASF |
| | | DWTVLEKEMSDFNKSKADDSKVEAEQKKLRDQIAKKLTSDKRFKALTASTPSDLFNKDKDFIDWFTQNSTK |
| | | DINKEALETFKRFSSYFKGFQENRKNVYSAEPIPTAVPYRLVNDNFPKFLQNIALFKIIQEKCPQVISDVEKELA |
| | | SYLGKEKLADIFTVQAFNKYLCQGGKENQRGIDFYNNILGGIAEKEGGINLRGINQFLNLYWQQHQDFAKQN |
| | | KRIKMIPLFKQILSDRSSLSFKIESINTDQELLTSITEYADKLETKSNDEKKSVLDICSDLFASIKAQNLQEIYVN |
| | | RKDINSISRILTGDWSWLQSRMNVYADEVFTTKAEKTRWQKSIDGDEGENKSKGVFSLAELNSVLEYSSENV |
| | | SETDVRITDFFDHRNRFYYEKESGLFKQGDELVALSIRESCEDILAKRKAMDEAFANVSENNSLRDNSEDVAK |
| | | IKIYLDCVQELLHRIKPLKVNGLGDPAFYAVFDTVYNSLSEVISLYNKTRNYITKKAANPEKYKLNFDNPTLA |
| | | DGWDLNKEQANTSVLMRKDGMYYLGIMNPKDKPKFAEKYECGNEACYEKMIYKQFDATKQIPKCSTQVKE |
| | | VKKHFQSGATDSIILNDKSKFKLDLVITKEIWFLNNHVWNGEKFVPKRESNETRPKKFQIGYYKQTGDLGGY |
| | | KEALNIWISFCKTFLQSYISSSIYDYDFKESSNYDSLDEFYNYLNATCYKLSFINIPEATISQMVSEGKLYLFQIY |
| | | NKDFAPGASGMPNMHTLYWKNLFSEENLKNVVLKLNGEAELFYRPAGIKEPVIHAKGSYLVNRITKDGEPIP |
| | | EKIHDEIYRNANGKLESLSKEATEYKASHKVVIKEAKHDIIKDRHYTEPKFLFHVPLTINFKASGNSYINENVR |
| | | RFLKNNPDVNVIGLDRGERHLIYLSLINQKGEIIKQFTFNEVERNKNGQVIKVNYHEKLDQREKVRGAARKS |
| | | WQAIGKIAELKEGYLSAVIHQLTKLMVEYNAIVVMEDLNFGFKRGRFHVEKQVYQKFEHMLIDKLNYLVFK |
| | | DRGLTEAGGVLNGYQLAGQFESFQKLGKQSGMLFYVPAGYTSKIDPKTGFASMSNFKDLTNVHKKRAFFSK |
| | | FDDIHFDDATGSFVFTFDYKNFDGKAKEEMKRTKWSVYSKDKRIVYLSKTKSYEDVQPTEKIKASLESVGIE |
| | | YMSGNNLIDSIMVIGAELKDGAKPSKEIADFWDRLLYNFKLILQMRNSNAKTGEDYIISPVMADDGTFFDSRE |
| | | EFKKGENAKMPVDADANGAYHIALKGLSLLKRFDAASENELKKFDMKISNVDWFKFAQEKSYAE |
| 23 | 278683 | MENMANSLKDFTNIYQLSKTLRFELKPIGKTEEHINRKLIIMHDEKRGEDYKSVTKLIDDYHRKFIHETLDPAH |
| | | FDWNPLAEALIQSGSKNNKALPAEQKEMREKIISMFTSQAVYKKLFKKELFSELLPEMIKSELVSDLEKQAQL |
| | | DAVKSFDKFSTYFTGFHENRKNIYSKKDTSTSIAFRIVHQNFPKFLANVRAYTLIKERAPEVIDKAQKELSGIL |
| | | GGKTLDDIFSIESFNNVLTQDKIDYYNQIIGGVSGKAGDKKLRGVNEFSNLYRQQHPEVASLRIKMVPLYKQI |
| | | LSDRTTLSFVPEALKDDEQAINAVDGLRSELERNDIFNRIKRLFGKNNLYSLDKIWIKNSSISAFSNELFKNWSF |
| | | IEDALKEFKENEFNGARSAGKKAEKWLKSKYFSFADIDAAVKSYSEQVSADISSAPSASYFAKFTNLIETAAE |
| | | NGRKFSYFAAESKAFRGDDGKTEIIKAYLDSLNDILHCLKPFETEDISDIDTEFYSAFAEIYDSVKDVIPVYNAV |
| | | RNYTTQKPFSTEKFKLNFENPALAKGWDKNKEQNNTAIILMKDGKYYLGVIDKNNKLRADDLADDGSAYG |
| | | YMKMNYKFIPTPHMELPKVFLPKRAPKRYNPSREILLIKENKTFIKDKNFNRTDCHKLIDFFKDSINKHKDWR |
| | | TFGFDFSDTDSYEDISDFYMEVQDQGYKLTFTRLSAEKIDKWVEEGRLFLFQIYNKDFADGAQGSPNLHTLY |
| | | WKAIFSEENLKDVVLKLNGEAELFFRRKSIDKPAVHAKGSMKVNRRDIDGNPIDEGTYVEICGYANGKRDM |
| | | ASLNAGARGLIESGLVRITEVKHELVKDKRYTIDKYFFHVPFTINFKAQGQGNINSDVNLFLRNNKDVNIIGID |
| | | RGERNLVYVSLIDRDGHIKLQKDFNIIGGMDYHAKLNQKEKERDTARKSWKTIGTIKELKEGYLSQVVHEIV |
| | | RLAVDNNAVIVMEDLNIGFKRGRFKVEKQVYQKFEKMLIDKLNYLVFKDAGYDAPCGILKGLQLTEKFESFT |
| | | KLGKQCGIIFYIPAGYTSKIDPTTGFVNLFNINDVSSKEKQKDFIGKLDSIRFDAKRDMFTFEFDYDKFRTYQTS |
| | | YRKKWAVWTNGKRIVREKDKDGKFRMNDRLLTEDMKNILNKYALAYKAGEDILPDVISRDKSLASEIFYVF |
| | | KNTLQMRNSKRDTGEDFIISPVLNAKGRFFDSRKTDAALPIDADANGAYHIALKGSLVLDAIDEKLKEDGRID |
| | | YKDMAVSNPKWFEFMQTRKFDF |
| 24 | 282110 | MVAFVDEFVGQYPVSKTLRFEARPVPETKKWLESDQCSVLFNDQKRNEYYGVLKELLDDYYRAYIEDTLAA |
| | | FTLDKALLENAYGLYCNRDNNAFSSCCEKLRKDLVKAFANLKDYLLGSDQLKDLVKLKAKVADSAGKSKK |
| | | KIEVDSRLLAWLKNNAKYSAEDRVKYCEAIEAFEGFVTYLTNYKQARENMFSSEDKSTAIAFRVIDQNMVTY |
| | | FGNIRIYEKIKAKYPELYSALKGFEKFFYPAGYSEILSQNKIDEYNYKCIGRPVEDADYKGVNSIINEYRQKKGI |
| | | KSRELPVMSMLYKQILSDRDNDFMSEVITSNEEAIECAKAGYKKSYILFNELLQLFNNSFIEENYEYIYIKTQPL |
| | | TELSQSLFGDWSVLRNALDNAKYDKEIINLAELVRYFSEYCQILDADDAVKIKNKFILEDYFVQDTLEAALPD |
| | | LDKITQYKTYLDNMLQVIRKYRLFSMYNGRKKLDVPEKGMDFSNEFNDIYDKLSEFSILYDRIRNFATKRPYS |
| | | DEKMKLSFNMPTMLAGWDYNNETANGCFLFIKDGKYYLGVADSKSKNIFDLKKNPHLWDKYSFENAFYKI |
| | | KYKQISGAAKMLPKTFLPSKGKPLSEEKKKKYGHLVTDRIIEIRDNGLYMKSAGDRKAVIEWIDFMKSAIAIH |
| | | PEWNEYFKFHFKNTEEYASANEFYEDMDKQTYSLEKVKIPAAYVDELVSQHKLYLFQLYTKDFSDKKKKKG |
| | | TDNLHTMYWNGVFSDENLKAIAEGTKPIIKLNGEAEMFMRNPSIPRKITHERNKPIANKNPLNNKKESLFSYD |
| | | LIKDKRYTERKFYFHCPITLNFRAAKPTKYNEKINRFVENNPDVCIIGIDRGERHLLYYTVINQTGDILEQGSLN |
| | | KISSSYTNNKGEKVNKETDYHALLDRKEKGKHAAQQAWETIENIKELKAGYLSQVVYKLTRLMLKYNAVIV |
| | | LENLNVGFKRGRTKVEKQVYQKFEKAMIDKLNYLVFKERGYEMNGSYAKGLQLTDKFESFEKIDIDKQTGC |
| | | IYYVIQSYTSHIDPKTGFVNLLNSKLKYESVSKAQETIRKFDRISYNAKADYFEFSFDYGKFGIDMFRNEWVV |
| | | CTCGDWRWEYSAKDRETKAYCVTDRLKELFKSHGIDYVGGENIVSKIADVADKRFLSTLLFYLRLVFKMRY |
| | | TVSGTDNENDFILSPVEYAPGKFFDSREAAATEPMNADANGAYHIALKGLMTIRGIENGKLHKFGKGGEKAA |
| | | WFKFMQNQEYKNNG |
| 25 | 288190 | MLNKFTNQYSLSKTLRFELKPVGETAGYLDDFKSQHLKEFVATDKQRADDYLIIKELIDDYHRAYIEEKLSAP |
| | | VDPKTGELFLNSQDFEDAYSYYQQLKVDSKDPKHRETWEKTQAELRKKLVKAFVGKADLFGEKLIKQHLPE |
| | | QLKKEGRWKENKKAVENFNKFTTYFSGFHENRKNMYTDKDQSTAIAFRLMNENLPRFFNNCDKYKKLKEK |
| | | YPELLFENNKDILAKLGVDSLNDVFTPSFFINLFTQTGIDNYQLLLGGKSEDTGEKLKGLNEQINLFRQQKQKE |
| | | AREQAQKENKKAKKITDLTGFTGLYKQILSDRQSASFILDEFENDRELLTKLNEFITLATKPEGTLEKLENALN |
| | | HLNEIDPQKVFIKTAGLTDISQTLFHSYQIITTALHEFAENNLKTKKERENYLKQKAFSLAELDERLVDYIQDL |
| | | EDSDPLHQQLQALNDPLQPIKGYLQEAITRAQQETDERPNLEQTIKEVEPLLTLKELSKNRQAPKKEGDKGGK |
| | | GFQQIQKIQKMLDAFMAVSHALKPLHLVDGRKPIDMPEMDSGFYGEFSEAYEEYNQSCITLYNKSRNHLTKK |
| | | PFKTDKIKINFDSPTLLKGWDANKESDNASLLFEKDGLYYLGIMHPKHKRLFDYIIGIDDIGNENKTKAKTDL |
| | | YNQITTNKSGYRKIVYKLLPGANKMLPKVFFSRSRIAFFAPSDEVQRIRNTASHSKNGKPQKGFKKADFNLKD |
| | | CHTMIDFFKDSINKHPEWKQFGFKFSPTESYEDLSGFYREVEQQGYRIDFHTIKESYIDECIKQGKLFLFQIYNK |
| | | DFSTYSKGKPNLHTLYWKGLFEAENLKDTVLKLNGEAEIFYRKHSIKPNDIITHRANETIENKSKNYDKKTST |
| | | FAYDIIKDKRYTQDKFQFHVPITLNFKAPKAPGVSHFNNQINKAVAKSKDTHIIGIDRGERHLLYYTVINQKGE |
| | | IIEQDTLNKIKAHKDYEVDYQQKLHDKETERQKARRSWSSVENIKELKAGYLSHVVHKLAVLIKEHNAIVCL |
| | | EDLNFGFKRGRFKVEKQVYQKFEKTLIDKLNYLVFKDAEENEAGHYLKAYQLTAPFESFEKMGKQSGILYY |
| | | VQAAYTSKIDPATGFINFLYTRYQSLSRSKEFFESFDFIRYNQAQDYFEFSFDYKNFPIRQNLKNYQTRWLACT |
| | | YGDERYANKRNKQNQWESEIVNVTQELKTLFNKASIDFQNGKDLKDAIAQTKDTKFYKKLYWLLGLTLSLR |
| | | HSKTGTEIDFILSPIQGKDGKFFDSREASNKHPKDADANGAYNIALKGLWNLQQIKQHDWNTEKPKKLNLN |
| | | MKNEDWFGFIAERASL |
| 26 | 298174 | MSQDNQKQFKTFQDFTNLYELSKTLRFELRPIGKTKELLEEQRVFQKDKKILENYKIAKKYFDKLHKKFINDA |
| | | LNNADLNFEEYKKAYFAYIKKNKDNRKKEFAELKKQETNLRKEVKSLFNHCAKSWKEKYNKVDVETKKDD |
| | | LGILFEKETLNILQYEFRAEQYPDVEYADEKGEKKNIFESFKGFTTYFTNFHNSRKNFYKDDGTVSAIPTRIIND |
| | | NLRKFIENSEIYSHTQNKIEFTNEEKKIFELDYYNKCFLQGGIDEYNKIIGDINSKINKYRQDKKDRQNKLPFLK |
| | | ILYKQILEDIGKQETKQNGFIEISNNGEVFEVLEKFIEHNENNNKKFKEIFSSFINNQNEYELDKIYLASRFVNTI |
| | | SNKWFNGWELFRGLILEKLNDNKSKSKQKKKLPYFIPFSQIKEILARTDIKAEDLFKKDILNKVGYTNQNVYD |
| | | VFISIWKNEFKENVKKYEETLSKVKAMIKEDKEYTNKKEKLENGVEVEVQKKKIENYADSALAIYRMMKYF |
| | | LLEKGKSSIDMQGDTDNKFYNDYDNAMENAKTWLYYNEFRNYLTKKPFNEDKIKLNFENGKLFDGWDQN |
| | | KEPDNFGVILKKDNRYYLGVAEKESANKIFRKKKGSSSEKIQEAYNFNGGYYEKMEYKQVPGLNKMIPKCLI |
| | | APYFKKRGNKKIIDRSKPGAKKLEKLGINPDDNFLLNYIENKHAKGDNFDREFFNEYVAFVKNSIPKYPDWE |
| | | CFDFNLKEIKDYDNLMDFYKDLERNSWKVWFEKVSEKYINEKIENGELYLFQIYNKDFSEKRTGNKNLHTIY |
| | | FESIFNEENIKDPVIKLNGQAEIFFREGNLKIEEELRTKKRNKKPHKIIKYKRYTENKILFHCPIKLNFAKSDNKI |
| | | NLKVNELLANNKDINIIGIDRGEKHLAYYSVIDQDGKIIDIGSLNKINNVDYLAKLDEKEKSIEKQQKSWQTIE |
| | | NIKELKQGYISQVIKKICDLAIKYNAIIVFEDLNSGFKRGRQKIKKQIYQKLELALINKLNYLVNKNEENGKAG |
| | | HFLSAYQLASKIDNFKDIGKQSGIVFYTQASYTSKTCPKCGFRKNITLKYINDKQVKDDLKKLKIKPVDDYFKI |
| | | EYKLSDFSENNKKKKNKDNELLAEYQRKNDFIIYSNVKRMRWHNINTKLAKRELDGEKIIKKFPPKGVIKEY |
| | | DINKCLKRMFKGKINIEKGDIIDQLIKANFNKDYYWELMYFLNLILEIRNSVSETDIDYIQCSKCGFHSDGGFQ |
| | | NKEFNGDANGAYNIARKGRLILEKINQHQDLSNLKTGDLAINIEEWDKAVNNWDKFVQK |
| 27 | 287476 | MKSLAQFQNLYALQKTLRFELKPEGHTRETFNRWLEEIEKEQASENENIVYQDLLRAKKYEKIKIILDEYHKD |
| | | FIEQALAYANLTELEKYEELYRKSNRTSEEEEEFENTKESLRKQIANIFIKNPNKTVQERWKFLFSKKLIQNELI |
| | | VWVKGNYELLSEKLKNEFPDESSIISTIEDFKYFTTYFRNYHENRKNLYSNEDKFSTIAHRLIHENLPKFIDNIAI |
| | | YQKAKAVLNINEVEKELGLPEDTLDKIFSLDFFSKALTQKGIDQYNYFLGGKTENEVKKIKGLNEFINLYNQQ |
| | | QQDKNQRLPFLKVLYKLPLFERTSTSFRFEPIENDRDLIERIGKFYYNDLKQYRDDSQGDTTDILSGINTLLRH |
| | | VHDYRDGLYVNGGITLTQISQKIFGSWSYINNALAYFYDTYIDASGVDHQGERKPKKQKQIQEKTKWLKQK |
| | | QFPVILVEKALSEYKSIETNEDLKTRISDTTLCDFFKRCGNDDNGQDLFDRIEARLREKNEEGYSLEDLLKKEF |
| | | TTERKLMQDKTKTLLIKNFLDVIQGDKDDITAGLLHFVKCLIPRTEISEKNELFYSGMEKYLNILSEVTPLYNK |
| | | ARNYLTQKPYSIEKVKLNFENSTLLDGWDENEESDNSCVLLRKRGYYYLGIMNKKHNMIFDRKIYPKATEGE |
| | | AYYEKMIYKLLPGAYKMLPKVFFSEKNIDYFKPSEEILRIRNTASYSKNGQPQEGYQKASFSIEDCRKYIDFFK |
| | | KCIANHWDWQKFNFNFSPTEYYQSIDEFYREIERQGYKIDFVKIPESYINQLIKENKLYLFKIYNKDFSEKKKS |
| | | KGKDNLHTLYWKMLFDEKNLKDVVLKLNGEAEVFFRQKSILYNEEIWNKGHHYSELKDRFSYPIISNKRYA |
| | | EDKFFLHVPITLNFKADGINNVNNMVNEFIKDNRDIHIIGIDRGERHLLYVSVINQKGDIVEQCSLNEIVTEYN |
| | | GKIFKKNYHEELDNLEKERDRARKDWQTIANIKELKEGYLSHVIHKISKLILKYNAIVVMEDLNSGFKRGRQ |
| | | KVEKQVYQNFEKQLIEKLNYLVLKESNVDEPGGVLRAYQLANKFETFKKLGKQSGIIFYVPAAYTSAIDPVT |
| | | GYIQYLYPLKQADSVEKARKFYSQFKRISYNPHKQWFEFSFDYNDFNIIYHGKSSWTICTTNTERYMWNRLL |
| | | NNGHGGEELVYVTNELELLFGEYNIIYGDGKDIKQQITDVQDIDVDRTAKQFYKRINELLNLTLKLRHNNGK |
| | | KGADEEDYILSPVEPYFDSRFESRKPSMQQTLPINADANGAFNIARKGLLLLERLNQLGVEEFEKTKKSNNKK |
| | | TQWLPHELWVEYAQNHTRK |
| 28 | 21528 | MLKSYDYFTKLYSLQKTLRFELKPIGKTLEHIKNSGIIESDETLEEQYAIVKNIIDKLHRKHIDEALSLVDFTKH |
| | | LDTLKTFQELYLKRGKTDKEKEELEKLSADLRKLIVSYLKGNVKEKTQHNLNPIKERFEILFGKELFTNEEFFL |
| | | LAENEKEKKAIQAFKGFTTYFKGFQENRKNMYSEEGNSTSIAYRIINENLPLFIENIARFQKVMSTIEKTTIKKL |
| | | EQNLKTELKKHNLPGIFTIEYFNNVLTQEGISRYNTIIGGKTTHEGVKIQGLNEIINLYNQQSKDVKLPILKPLH |
| | | KQILSEEYSTSFKIKAFENDNEVLKAIDTFWNEHIEKSIHPVTGNKFNILSKIENLCDQLQKYKDKDLEKLFIER |
| | | KNLSTVSHQVYGQWNIIRDALRMHLEMNNKNIKEKDIDKYLDNDAFSWKEIKDSIKIYKEHVEDAKELNEN |
| | | GIIKYFSAMSINEEDDEKEYSISLIKNINEKYNNVKSILQEDRTGKSDLHQDKEKVGIIKEFLDSLKQLQWFLRL |
| | | LYVTVPLDEKDYEFYNELEVYYEALLPLNSLYNKVRNYMTRKPYSVEKFKLNFNSPTLLDGWDKNKETANL |
| | | SIILRKNGKYYLGIMNKENNTIFEYYPGTKSNDYYEKMIYKLLPGPNKMLPKVFFSKKGLEYYNPPKEILNIYE |
| | | KGEFKKDKSGNFKKESLHTLIDFYKEAIAKNEDWEVFNFKFKNTKEYEDISQFYRDVEEQGYLITFEKVDAN |
| | | YVDKLVKEGKLYLFQIYNKDFSENKKSKGNPNLHTIYWKGLYDSENLKNVVYKLNGEAEVFYRKKSIDYPE |
| | | EIYNHGHHKEELLGKFNYPIIKDRRYTQDKFLFHVPITMNFISKEEKRVNQLACEYLSATKEDVHIIGIDRGER |
| | | HLLYLSLIDKEGNIKKQLSLNTIKNENYDKEIDYRVKLDEKEKKRDEARKNWDVIENIKELKEGYMSQVIHII |
| | | AKMMVEEKAILIMEDLNIGFKRGRFKVEKQVYQKFEKMLIDKLNYLVFKNKNPLEPGGSLNAYQLTSKFDSF |
| | | KKLGKQSGFIFYVPSAYTSKIDPTTGFYNFIQVDVPNLEKGKEFFSKFEKIIYNTKEDYFEFHCKYGKFVSEPK |
| | | NKDNDRKTKESLTYYNAIKDTVWVVCSTNHERYKIVRNKAGYYESHPVDVTKNLKDIFSQANINYNEGKDI |
| | | KPIIIESNNAKLLKSIAEQLKLILAMRYNNGKHGDDEKDYILSPVKNKQGKFFCTLDGNQTLPINADANGAYN |
| | | IALKGLLLIEKIKKQQGKIKDLYISNLEWFMFMMSR |
| 93 | 2237668 | MPQSNSQQKKPSYNGRTGYALHTARLSYVGKEGMRSAKIPLDGEDSTRNTLWDNVRADYIGQVGEMNIGDW |
| | | YEGEQKGAHVYALADFWLDSLRAGVIFSHKEKEMHGVIAHLNGDETFSKKFSLALAERNPELAALADNTALY |
| | | QFFISGKRPGKKTDEEFFWSVRNSVFPRFRNVEDATVDAYARRLVDTVLKQTKDDTEADTLKIVLAKEYVGF |
| | | DPTPDIPYKHTCFFHPQTSLLPDASAKQILAMYKELATQKDPTLYGYDGENESDVMKQIFGLENNQGAFANYF |
| | | NTTLADFRAGGEHTEQAIVTVSPYWATHRNELHARIMFLVERARMLPEPHLTKGWHDYRSTFGGKIESWVSN |
| | | VLRQEKEIAKQLFGGIEESGKGARKKETFVRGHKQILEEVLADTCLSDDTKAIAHECFDLVLRMEISGITDADL |
| | | LLYRTMLGDARIGMNVDYQTAYPELKGKTEKERKQEEKEKRAYRKYATLYTNIKLIPNFLGDSKRIVYKKFIQ |
| | | SAEILHTSIATLASLEMTLSEAPISPKFTSDDEAEIFVTKLYETLRKKYAVLNATRFRGVIEGIFRHLKGTLNGEM |
| | | FDMMNVLERADETESTQKIEYVCYTPPYARVHPRRRTVDIIIKNEDIPALLDTLLVALKPRWDDIMAGGDIGEL |
| | | ADAVEMEKIRMGIVVALWYRHPVARILDELSEELFSSAHAFAKLKGNPETFSGEDLGRLLQSGILSEMKGAITT |
| | | MSRVEYTERYTGQLMNTETHMPLCRTEDGWHILSGKYQKKEKDGMIAVPIKKGKGFEGETDYTTEYIHTQDL |
| | | LHLNTSVYQLQFLDKTLPQGKRTISWWDAHDIAFSLNSYSFIAERNMRIDWDISQKKFRITPGEEKRLFVSIPFS |
| | | FSPKNKATDTLEKRTRYMGIDVGEYGLAWTIIETDAVHKKVVRILKQGFIYEPLTHTVRTYVATLKNLQTKGT |
| | | FGMPNTKLERLRENAVGSLRNKVHDIAMRYDAIPVYEFEISNFETGSKRTTVIYNSVKRADVGRGGSEADKAE |
| | | ASLVWGKTGKNFGKEIGAYATSYICSRCGYSPYEASVIEGTRATLDMEKKLSRPSLNDFLKDHSAYKTREEFA |
| | | MRGNKESIWKERRGNSAIYVCQHCGHVTDADVQASYWIAVKSFAKSLRSKKEEKDNSPVSFEEMSAIHDISSG |
| | | TTADNKRITRLRIDI |
| 94 | 2236875 | LDPAVIATIDDLIETFFTSNGDTIERDKQNQYWRDQWGLDKSLYDLDGDKDLTFYPLPKLEMTPDAGAHDCLR |
| | | KYRNWIDDNQDTFGLDDQEAKEAFLRLCWGNLQASGNYNAFSNYFNQIVNTLQSEDGTDDVVDDLCTASDV |
| | | WSGQENHLRERIAFLADGARRLGQPKCVADWSKYRSTLGGRLASWLSNTIRQEEFLAETTQKQVEELTDIATR |
| | | LDNEFYAEAFSAADDTRDRQLASEVETAQDTLRKLRDNEGENPDALSVYRDSIGRLRSLLNRAHQILGDDTVD |
| | | AYEAYPALHEQVRLVPKFVGGAKEARFSKYRNSLHMLKRGVTFLQEEAPDAPDAAGVRNSDEEFESYFLRQL |
| | | NALQRFYQDANSQRFRDIVSDLFARFDIDISAVSSAKTFYISPYSNRDNRQVINVDTDNKDVSNLLARWANEIK |
| | | PYWDDILATENWEEIMDAVKIDRIRYGWIGRLSADTTIHIADDLSDHFPEAETYRDLHGDTLSGTQADNFLQR |
| | | VILSEMEGRVNEASREEFTVRYVVQPQDAGKKSNYPLVVSSNGGDKITAGTRMQWYIKDNDTSFAHSETNRS |
| | | VWTLSDSKTFANGLEKKYVKPQTNLFAIKTSKYQLQFLDNALPGGAKRFRDDGGFDLELQDHSFIAEETLSVN |
| | | WNFDTGNLHLESTDKNMYAAIPVAVIPENEDSDRDNRTRYLGIDVGEYGLAYTIIETDPEVSILKTGFEHEPTL |
| | | RNIRKQVTNIKSKQRTGTFGVPSTKLARLRKQAVTKLRNRMHDLAVRFDAIPVYEWQISNFETGSARVTNVY |
| | | NSVKRADTSSGMGSDAESGEAELVWGDSSWRVGREVSAFATSNMCSNCFESVYAYPDEAFSLQSVESEYAY |
| | | YTVDGTQENVMAFLSDTDPEGISGNKLKQAVKDFSRPSIDTLVERQTNDFDLSKGKTTELIRQRGNSALFECPF |
| | | CHAVNDADIQASLFIGLRAILKDRTGEDEEITIPGLLTFAESADV |
| 95 | 2236834 | MNLFMTEDTNRKKYKGITGYSLHNKRLEYTGKSSIRSIKYPLIPNNWQIYHSFCDDIINDYERINGAVNITTYAK |
| | | NPKIYSLFDFWIDSLRTGVIFRESANSLKNSLEKLRSKKSTAKISFDLMKVPAKKKLDSELFINKFVLSTGVRSTS |
| | | TKQSLENKLLGCFKEKYRTNADVKKIAKKISSDLLPNRKALSQTEQKKFWHENYNLQIPAKKEKSSRNLTFYIL |
| | | PELELDKNLSVLGCIKRLKSIFDKENAKYLGLTSNFNAFSNYLNEVFSLFYDNKLKRISAALKEISKDWEGRED |
| | | GLIKRLEFLSQKSRLLGKPRLTKSWADYRSVWGGKLQSWYSGFDRQASLIRKAIKDNESELTKLKKEVVKPRS |
| | | SKKKRYLFQKKNLTRLIDHLLKLSPKIKSKNNLEFAQIDLYRDLLSQLRINLNEFYQKFLQTKKDKKEGKAVD |
| | | KVYKSLFKNLRLVPDFFGESKKKNYEKYIFKTIPIIKQGIAITHTIEGQLLRGVDFSLVTQDYFYRQLEKINCKLR |
| | | SNALNSSQKIHQFQNIIKTYNKGILSKKDEVFYKSQYSNNKKLKLIAWKNLNYGEELKKIYFKLKEEYSLNKSP |
| | | DKLLDYIEILKIRIGWLLFLSSIRINTSEFDFSLFEKAKTYSQNFGSILKGEDRSFFIMSLILSEIRGAASLFSRKFFT |
| | | ARYVAQIINSEKKFPLIYIPKKKNDKNITLPSRINRMPDSNKKLLNKIDTSLMFFYPHQWGVVIPRNNKEITNGK |
| | | ESIKTIDFTGKNSQGLKVDEHSFEQIFNIYSSKYQIHFLDRLLYKTKKWKKISLALSEPFLIVEEEVKVKWNLVT |
| | | SKPELLTDKNKKLFISIPFNLNPETKWRNQNKIIEKRNNYIGIDVGEYGLAWAVVSFREKNPKIIDKGFVFDPVV |
| | | RKIADRIQDIKESQTKGTFGVPSTALARVRENAIKSLRNKIHYLALKYNAKIIFEYQINNFETGSNRVSKVYRSV |
| | | KVSDVKLENKADELVKNQVWGNYSKYIGNHISAYATSYTCTKCYRSIYDECSKDKRYLIVKKWKNILDIKINK |
| | | SLIAKGYTKKQQINTGDKLSYKVTMNALKGYARPPANSEALSEINIQEKDLQKFVTKRGNCAIFICPFCGHVAD |
| | | ADIQAALNIACRGYLRDNKTKKFDYLEAMKIYMKNVKFPIVSLEYIQPTLIDKKIRLTEKPKQKELRIKHS |
| 96 | 2236172 | VKKKYKKHKGITGYTLHNKRLVYSGNSSIRSIKLPLKFNGNPQILQDYYDSLIYDYEKMNGPVNISTFFKDPSL |
| | | YSLFNFWLDCLRVGVIFASSTASLQDTVYQVFGVLSPTQKAIKAIPEHIRKELNEKNFIEKVLLSEGMRSRTEKD |
| | | SFINKMKKCFDIPQNTPEQELPGEKLIILETIQKIAEKMFVNGQVVDYKTQKDIWKEELNVEFPQKPTSKSTFYII |
| | | PKLLDRSKYSDLVDLEHITPIDLINNFKSFVSETWGLDEENKAVTDKTKSLVGLDSKFNGFSNFLNFVFKLLLD |
| | | ADIEQIKQKLNEIFDLSHAWNDNITDLDERLIYLSQKAKKLGKPKFRNGWHEYRSVFGGKLSSWFSNLLNQEK |
| | | EIREQLIKHREEIEEVLKFVINNDSSSSIVTTLDSLKGLSETILNSENILEKEKEINLYREILSEFKFNFNYFYQKNS |
| | | DKKNSEKDVKKAYPNLFKNLRVIPNFFGGSKYEQYDKYVYKTFKIINEGTQITLDLEKILLKNLETNNENIQID |
| | | KEYFQKQLQTLLNKIKTDALEYKEIKNIVELILKKYNNGNLPKEFEAFYVSPYSRDKRLKQINLPDELDFTQELL |
| | | NVHKKLSSFNVGEFNADIVKFLSFVEILKIRIGWVLNLTKNNDLDVSRYDFTQFEKAKTYVDNFGTILKNDQR |
| | | NIFINNFILSEIRGALSKASRKEFTERFVIQYVDSEKKNLLVYVPNIANNKNIITPHGVTIPKNMNELLSEKNKET |
| | | KNFRRSLFFYPHRWKVSLFELKKVKNSNFNALSLQKKNFTNLVPVNIDDDNTFDICTSKYQIHFLDRLLYQTK |
| | | KWKRMHIEFSSPALILEVKNNVKWDLETQRPHLSIKQQVLYINYPINLKAEESKNHIQTQAKNYLGIDTGEYGL |
| | | AWVALDFSDLSAPKLLDKGFIYDDSLRKIRDRVDEIKDTQIKGTFGIPSTKLARIRKHTITTLRNKVHDLLLKYN |
| | | AKVIYEYSISNFETGSQRVTKIYRSIKVSDVFAETDADKSVKKLVWGKENFNTGNHISAYATSYTCSKCCRSIY |
| | | FEQEKQNENVVVEKDNNLLKVKLREGVFVWAYSSDKSISEGSVVKNEDLIKMVREYARPPENSEALKRVNPK |
| | | INDIFEKRGSSVIFICPYDDCNHLSDADIQAAQNIAIKGFLSDLRNSPKRNSAKINDEGKLQEFIEYIQNPKNKVPI |
| | | IELIE |
| 97 | 2237025 | SRPSDVTGIMANRQISPVDSTNNFIFTLYVSFDTVLYARILAFLTLSCLLVGFLEYIYPLGVIYLIMPTNQQYDGL |
| | | TGYQLHTERLRHTGKSGIRTFKLPLKTNDQELFGNFFENIKHDHEGQIGATNITTWTEEQKSSRQIYSLLDFWL |
| | | DSVRAGVVFASSSAELNELLSVCGDEDAVDEQVKTAMRAINPTFFKHLKFNDFKEEIALKEGMRSSSTKNSVT |
| | | RRLYKCLDCTEEDAPEEIQQAVADLIKTFFTTDGKIISRNDQDDYWQVHFGLDKSLYKLDSDIKLTFSFLPDIPF |
| | | TPEADAHMCLDKYRCWIDENAEKFQLESKEDKTNFLQIHWGIEGNYNAFSNYFNQIIDLLREEEGRQKLLDAL |
| | | LNTSDLWEGAETELKKRFDFLVDKAKQLPKAKCVDSWSDYRTTFGGRLSSWLSNTLNQEEFIKDTVSDQKEE |
| | | LKQIIKNSEINLYKKAFSAADDNRAVELSHEAVIAQKTLEKLQKSSSFEPQLLGVYRDNLGRLRSLLNKAHQLL |
| | | PDEIENKSAHEVYSALHERIRLMPKFIGGAKAARFEKYVKSLQILKQGASFLENFEEKVKEAMKSSVSVEVEEI |
| | | SEGYFLRQLNTLKRFYDNANDSRFKGKLSSLFDKDLGVNIEAVSNRETFYISPYSKHDNRAVISVEVANYQQL |
| | | LKSWVNELKPYWGNIIATENWGEIIDAMQLERIRIGWIYKLYPKLTFAISDDLDELFAKAATYRDLHGHEVEGS |
| | | LAANFLQRIILSEIEGRVKEASREKFTVRYVIQPVGSEKTTKYPLFVTSNKDKLSATERKQWYIRDINSSFSRSET |
| | | DKKGWILKNGKDFSNGFQEKYISDNTDLFKIISSKYQLQFLDKALPNGAKKFGKDGDVNLKLSEHSFIAEETLQ |
| | | ASWDFSTDTVSLKSVDKDLFVAIPVNIEPESQRSSREERTRYLGIDVGEYGLAYTLIETIPKVRVIKSGFLYEKTL |
| | | RNIRKQVTNIKNSQRLGTFGVPSTKLARLRTQAITKLRNKVHDLVVRYDAIPVYEWQISNFETKSGRVTKIYNS |
| | | VKRADTNSGANENADGDEAALVWGKRPYKIGQEVSARATSNMCSNCFKSIYQFTEQDFTEESRSGEYVEFKI |
| | | AKNTVKGFVPNDEKITSKQKMINAVHDYARPSVDILVKRNDVGVDKEILVNFLEHRGASALYECPFCSHLSDA |
| | | DVQASLFIGLRAICRDQDDETTKVKISDLMDFAQKQNIDPISADFIQ |
| 98 | 2235244 | LKRIAKFRHDKPVKREAWSKGYRVHKNRIINKVTRSIKYPLVVKDEWKKRLIDDAAHDYRWLVGPINYSDW |
| | | CRDPNQYSILEFWIDFLCVGGVFQSSHSNICRLAIQLSGGSVFEQEWKDLSPFVRANLIQGIKPAEFIGFLTAEFR |
| | | SSSNPKNFISKFFEGSNEDLESLTNEFASIVDFIKAKDISLLRKSLPSCKKIAPNLWEKAVGSHSTNELLKLLTKY |
| | | TRVMLVAEPSHSDRVFSQTVLQSNDQDDPELTGPLPSHKVGKASYLFIPEFIREVNLDKISKLDLSAKSKLAVE |
| | | QVKKLSELTSDFKQIENQSEAYFGLSTSFNELSNFLGILIRTLRNAPEAILKDQIALCAPLDKDILKITLDWLCDR |
| | | AQALPENPRFETNWAEYRSYLGGKIKSWFSNYENFFEIPQAASSQQNNNREKKLGNRSAIRALNLKKEAFEKA |
| | | RETFKGDKGTLEKIDLAYRLLGSISPEVLQCDEGLKLYQQFNDELLVLNETINQKFQDAKRDIKAKKEKESFEK |
| | | LQRNLSSPLPRIPEFFGERAKKGYQKARVSPKLARHLLECLNDWLARFAKVEESAFSEKEFQRILDWLRTSDFL |
| | | PVFIRKSKDPPSWLRYIARVATGKYYFWVSEYSRKRVQIIDKPIAQNPLKELISWFLLNKDAFSRDNELFKGLSS |
| | | KMVTLARIMAGILRDRGEGLKELQAMTSKLDNIGLLHPSFSVPVTDSLKDAAFYRAFFSELEGLLNIGRSRLIIE |
| | | RITLQSQQSKNKKTRRPLMPEPFINEDKEVFLAFPKFETKNKVKGTRVVYNSPDEVNWLLSPIRSSKGQLSFMF |
| | | RCLSEDAKIMTTSGGCSYIVEFKKLLEAQEEVLSIHDCDIIPRAFVSIPFTLERESEETKPDWKPNRFMGVDIGEY |
| | | AVAYCVIEKGTDSIEILDCGIVRNGAHRVLKEKVDRLKRRQRSMTFGAMDTSIAAARESLVGNYRNRLHAIAL |
| | | KHGAKLVYEYEVSAFESGGNRIKKVYETLKKSDCTGETEADKNARKHIWGETNAVGDQIGAGWTSQTCAKC |
| | | GRSFGADLKAGNFGVAVPVPEKVEDSKGHYAYHEFPFEDGLKVRGFLKPNKIISDQKELAKAVHAYMRPPLV |
| | | ALGKRKLPKNARYRRGNSSLFRCPFSDCGFTADADIQAAYNIAVKQLYKPKKGYPKERKWQDFVILKPKEPS |
| | | KLFDKQFYRPN |
| 99 | 2236994 | MLPFGAGALIIILNMQKGYNLHKKRIDLLNGEILRTIKYPLKPLNENSEKNKKQFELQIINDDLKIRGDLNLNDY |
| | | LNYTFKNNHIYNLFDFWIDSLKCGVIWQPSFNNLKNLIEKIYLNKTISEEIFDNSIPEIKKFFDKNKFIENFILRTGI |
| | | RENKSKKSFEKDLIKSLNDEFNKEEINKEAQDLIDKIINTFFNENKLKKLTIEEQNNLWLNKFNIDKSKLKTKEL |
| | | KYNYTFFIIPELIKFKENNLENLLNIRYEFIKNKNLNIEILQEILGLENNFNSFSNYFNEIIKNLQNDEYNLIYEDFIS |
| | | FYKFTNEEKEKILQSLKYLSEKSKQLGSPQILNLSWADYRSILGGKIQSWFTNYLQRKEKLEEQLNSLKENINK |
| | | TKEYLLENFQENTDLKEIIYFLDKLFELSEVSKINNLFFDIHSTCKLKINNFFQKNIKQNEDDTVESNENLKGIFE |
| | | KIYKPLNFYGQSRRENNIKIINSLQTVELGIIYTKKILNFLKNDLDFNKEENFEEIYRKNLNFLWNKLKENSLNSK |
| | | NFKSKYKEIIIKNLNSYSNIKFEKIIKRPSYYVFYKSIYSKGTNLQIEFNKEINFKNIFIENINELYNFLINLEDKEIIT |
| | | DKNLLLDWIELSKNISSYFIKFTNIEKINLKEIFKDKDLNFFSNIAKYFETFGNVVEKQGLSYFVQSIFFSEFKGAA |
| | | TLYTKNKYLAKYNIQIIHSNEKFPLYYYSNNLKNHNKLLNPKDLIREKHNYFVSLKKIKILKNKDLEFLLLNKD |
| | | NFQKIYNSFEDNKNDLLKLESSFYQIQFLEKFIYKPKNWENSKIELSEWSFILEKEYEIKWDLENKIPILNEIQNS |
| | | KKNKLYIAIPFKISSYQKEYEEIKKSKENYILGIDVGEYGIGWVLINYKNKELKIINKGFFVSKNIAKIKDEFSKIQ |
| | | KKSQKGIFNESSNLISEIRKNAIGELRNKIHSVFIFYHSKNICYEYSINAFETGSGKTTAIYKSIKKSDTLSIIDADK |
| | | KEQMHIWGKKLNKEIEIGKNLSSYASSYICSKCGKSLYQLKETDLENNEIKIIEREGNILKIKTPYGNVYAYSLK |
| | | KEHKVDFIFNKTKSSFKELQKIIKDFARPPLNNNSEVIRKFTNLKENEIDILHQKRGSSCVFVCPFCLHVSDADIQ |
| | | AALMMSIRGVINNETKSKGEKIDYFQKNLEFLNELKEKPVIDL |
| 100 | 2237634 | MGQRGERCVRRKKSFKGYRLHAERLALASEASNLETDTIRTIKFPLVGTDPLVKDAFAAVRHDYLAIHGPLTF |
| | | HDWVGSQGRESYSLVDFWIDAVAIGGVFGASASKLRQLAAELAGEAPVEAFKARYAAAVNPALEHLEDWAK |
| | | IAEFFSKDLRGRAEGCFRKGFRDEPAAREVLSRYNQARKELRQSESYRSSEDKGALLWECRARALFGFSAPQD |
| | | QFPSRPGGATEDSLGAEMPDLKLTFLLVPSCIQESVGGGKCSAPWRIDRTEPSFSLQQGRTSAVLQLLGFVEGL |
| | | KGGAGLSEEKDDLLGLTDNYNALQNFFNVFLQELREDPSSVARAMVCLYPAKLHDSIRDEIVSKCEALSRLAQ |
| | | RLAQPKLAANWSEYRSFLGGEILSWVSNQKRLIGELTTGLQSKVEALEEVKGRLAEQRFTGALSQPKWQLLPS |
| | | QIAEVIQVLHGLQASLGAGGVVPRDFARTFDLFQDLLASLNADLNRFDQLHGLFNRASRQRAGRPGPRAARST |
| | | AQDDAEQTDSLQSKLSKRVQQVPAFFGETKKAHYPKFRAAPALISWFVDTLKTAFAAAQVTKRPLTPEEVER |
| | | MLRWLADTAEYGSLEEKILKAETCPLRHLLQLRRFPRSSRPYCYKSRYVRQRREPALSAEQFSAWMGTSPELS |
| | | ADGVVAWFADEVNRTLSGVKGGRNELLPLFRALIKLWPGVIRLGGNPSADVRERLLPRLVEFFGSAKHGEMR |
| | | KESFPRLSAYLDFVGALSGECSLEDAQADRILSKLLAECSGLLALASYSSFIARYSLQHQKGAQTNLLVQGAED |
| | | PRTARFSMELVGLRPASGGVAHANAHAPDLRLLMKAQVFNRNTSPFRGGGAVLSIQSSKYQVQFLRRALKEA |
| | | QAGAWAVAPSGSFLIAETDVRVRPNWTRGEMEVITDEAALPRLYSSIPFNLSVKRDPAPEPEMFVRPGGANDE |
| | | RGRVIGVDIGEYALTFSVVEWSRAGERVQFRVLDTHERVLKDGAHRVLKDEVRRLKKRQVRGTFSTRSSAVA |
| | | DLRETLIGAYRAKLHDLVVRYRAPLVFEFNVSAFEAGGNKIKKIYDSVKISDTGGDSDADKNVRKHYWGKKF |
| | | QKGFVTGREIGAKHTSQLCSSCRRVALEELDELLRSESFEDKKELDAHGRYLWHEVNVGGTKFRAFLGRDESL |
| | | SKPESVKRFVRAALRPPLKRFQRKLPDAARKRRGNSALFWCPFIDCNHLSDCDVQASVVIAVKAVLNQLFKEE |
| | | YKKGWLKLVQDRARVELPVVALEARYFRNPYAPREAEGLN |
| 101 | 2237034 | MTKDNSQHNGLTGYRLHTKRLGHTGNAGIRTFKLPIHAEDNSRHDSFFADIKNDHEKQLGATNITSWLESREG |
| | | DADMYSLLDFWLDSLRAGVVFAPKSRALNKLLADIAGSSIDEQVKASMREKQPAFVQAVNFADFKEKFALKG |
| | | AIRSTSTKDSVRRRFLQCFDEDEDDLDPAVIATIDDLIETFFTSNGDTIERDKQNQYWRDQWGLDKSLYDLDG |
| | | DKDLTFYPLPKLEMTPDAGAHDCLRKYRNWIDDNQDTFGLDDQEAKEAFLRLCWGNLQASGNYNAFSNYFN |
| | | QIVNTLQSEDGIDDVVDDLCTASDVWSGQENHLRERIAFLADGARRLGQPKCVADWSKYRSTLGGRLASWLS |
| | | NTIRQEEFLAETTQKQVEELTDIATRLDNEFYAEAFSAADDTRDRQLASEVETAQDTLRKLRDNEGFDPDALS |
| | | VYRDSIGHLRSLLNRAHQILGDDTVDAYEAYPALHEQVRLVPKFVGGAKEARFSKYRNSLHMLKRGVTFLQE |
| | | EAPDAPDAAGVRNSDEEFESYFLRQLNALQRFYQDANSQRFRDIVSDLFARFDIDISAVSSAKTFYISPYSNRDN |
| | | RQVINVDTDNKDVSKLLARWANEIKPYWDDILATENWEEIMDAVKIDRIRYGWIGRLSADTTIHIADDLSDHF |
| | | PEAETYRDLHGDTLSGTQADNFLQRVILSEMEGRVNEASREEFTVRYVVQPQDAGKKSNYPLVVSSNGGDKI |
| | | TAGTRMQWYIKDNDTSFAHSETNRSVWTLSDSKTFANGLEKKYVKPQTNLFAIKTSKYQLQFLDNALPGGAK |
| | | RFRDDGGFDLELQDHSFIAEETLSVNWNFDTGNLHLESTDKNMYAAIPIAIIPENEDSDRDNRTRYLGIDVGEY |
| | | GLAYTIIETEPEVSILETGFEHEPTLRNIRKQVTNIKNKQRTGTFGVPSTKLARLRKQAVTKLRNRMHDLAVRF |
| | | DAILVYEWQISNFETGSARVTNVYNSVKRADTSSGMGSDAESGEAELVWGDSSWRVGREVSAFATSNMCSN |
| | | CFESVYAYPDEAFSLQSVESEYAYYTVDGTQENVMAFLPDTDPEGISGGKLKQAIKDFSRPSIDTLVERQTNDF |
| | | DLSKGKTTELIRHRGNSALFECPFCHAVNDADIQASLFIGLRAILKDRTGEDEEITIPGLLTFAESADV |
| 102 | 2236881 | MGQRGERCVRRKKSFKGYRLHAERLALASEASNLETDTIRTIKFPLVGTDPLVKDAFAAVRHDYLAIHGPLTF |
| | | HDWVGSQGQESYSVVDFWIDAVAIGGVFGSSASKLRQLAAGLAGEDPVEACKGRYVSGVGPALESTLDWAQ |
| | | VADFLGKDARANVKWCFKEEFREAPAAHDIIARYNQAKTELQQSEPYRSSEHKDVLLRECRERALFGFSVPQ |
| | | DPLPLVPGSVAEESLGLEAADLRLTFLLVPSFIQDSVEGGKCSAPWTVDSTDFSFSLNQGRTQAVLQVLGFAKR |
| | | LKEGARLSQGEEELLGLTDNFNAFSNFFNLFLHKLRADPSSVARMMVSLYPARLHDSIRDEIVSKCEALSRLAQ |
| | | RLAQPKLATNWSEYRSFLGGEILSWVSNQKRLIDELATGLQSKVEALEEVKGRLAEQRFTGALSQPKWQLLPS |
| | | QIAEVIQVLHGLQASLGAGGVVPRDFARTFDLFQDLLASLNADLNRFDQLHGLFNRASRQRAGRPGPRAARST |
| | | AQDDAEQTDSLQSKLSKRVQQVPAFFGETKKAHYPKFRAAPALISWFVDTLKTAFAAAQVTKRPLTPEEVER |
| | | MLRWLADTAEYGSLEEKILKAETCPLRHLLQLRRFPRSSRPYCYKSSYARQRREPALSAEQFSAWMGTSPELS |
| | | ADGVVAWFADEVNRTLSGVKGGRNELLPLFRALIKLWPGVIRLGGNPSADVRERLLPRLVEFFGSAKHGEMR |
| | | KESFPRLSAYLDFVGALSGECSLEDAQADRILSKLLAECSGLLALASYSSFIARYSLQHQKGAQTNLLVQGAED |
| | | PRTARFSMELVGLRPASGGVAHANAHAPDLRLLMKAQVFNRNTSPFRGGGAVLSIQSSKYQVQFLRRALKEA |
| | | QAGAWAVAPSGSFLIAETDVRVRPNWTRGEMEVITDEAALPRLYSSIPFNLSVKRDPAPEPEMFVRPGGANDE |
| | | RGRVIGVDIGEYALTFSVVEWSRAGERVQFRVLDTHERVLKDGAHRVLKDEVRRLKKRQVRGTFSTRSSAVA |
| | | DLRETLIGAYRAKLHDLVVRYRAPLVFEFNVSAFEAGGNKIKKIYDSVKISDTGGDSDADKNVRKHYWGKKF |
| | | QKGFVTGREIGAKHTSQLCSSCRRVALEELDELLRSESFEDKKELDAHGRYLWHEVNVGGTKFRAFLGRDESL |
| | | SKPESVKRFVRAALRPPLKRFQRKLPDAARKRRGNSALFWCPFIDCNHLSDCDVQASVVIAVKAVLNQLFKEE |
| | | YKKGWLKLVQDRARVELPVVALEARYFRNPYAPREAEGLN |
| 103 | 2237533 | MANRQISPVDSTNNFIFTLYVSFDTVLYARILAFLTLSCLLVGFLEYIYPLGVIYLIMPTNQQYDGLTGYQLHTE |
| | | RLRHTGKSGIRTFKLPLKTNDQELFGNFFENIKHDHEGQIGATNITTWTEEQKSSRQIYSLLDFWLDSVRAGVV |
| | | FASSSAELNELLSVCGDEDAVDEQVKTAMRAINPTFFKHLKFNDFKEEIALKEGMRSSSTKNSVTRRLYKCLD |
| | | CTEEDAPEEIQQAVADLIKTFFTTDGKIISRNDQDDYWQVHFGLDKSLYKLDSDIKLTFSFLPDIPFTPEADAHM |
| | | CLDKYRCWIDENAEKFQLESKEDKTNFLQIHWGIEGNYNAFSNYFNQIIDLLREEEGRQKLLDALLNTSDLWE |
| | | GAETELQKRFDFLVDKAKQLPKAKCVDSWSDYRTTFGGRLSSWLSNTLNQEEFIKETVSDQKKELKQIIEKNL |
| | | YKKAFSAADDNRAVELSHEAVIAQKTLEKLQKSSSFEPQLLGVYRDNLGRLRSLLNKAHQLLPDEIENKSAHE |
| | | VYSALHERIRLMPKFIGGAKAARFEKYVKSLQILKQGASFLENFEEKVKEAMKSSVSVEVEEISEGYFLRQLNT |
| | | LKRFYDNANDSRFKGKLSSLFDKDLGVNIEAVSNRETFYISPYSKHDNRAVISVEVANYQQLLKSWVNELKPY |
| | | WGNIIATENWGEIIDAMQLERIRIGWIYKLYPKLTFAISDDLDELFAKAATYRDLHGHEVEGSLAANFLQRIILS |
| | | EIEGRVKEASREKFTVRYVIQPVGSEKTTKYPLFVTSNKDKLSATERKQWYIRDINSSFSRSETDKKGWILKNG |
| | | KDFSNGFQEKYISDNTDLFKIISSKYQLQFLDKALPNGAKKFGKDGDVNLKLSEHSFIAEETLQASWDFSTDTV |
| | | SLKSVDKDLFVAIPVNIEPESQRSSREERTRYLGIDVGEYGLAYTLIETIPKVRVIKSGFLYEKTLRNIRKQVTNI |
| | | KNSQRLGTFGVPSTKLARLRTQAITKLRNKVHDLVVRYDAIPVYEWQISNFETKSGRVTKIYNSVKRADTNSG |
| | | ANENADGDEAALVWGKRPYKIGQEVSARATSNMCSNCFKSIYQFTEQDFTEESRSGEYVEFKIAKNTVKGFVP |
| | | NDEKITSKQKMINAVHDYARPSVDILVKRNDVGVDKEILVNFLEHRGASALYECPFCSHLSDADVQASLFIGL |
| | | RAICRDQDDETTKVKISDLMDFAQKQNIDPISADFIQ |
| 104 | 2236715 | MSVARSKIVDWRGRTGYALQSHRLLFAARVSRDKKIGTVRTIRFPVKVEDRRISSWLTGSAISEYNTLIGSPLN |
| | | ELLSATSNGAGRITTLLDFWLDALRMGAFFQPSARRLTDLVFIFAGRGKDSPAELIWRSVPARLRKVLRRKVFI |
| | | DEFVLESEMRRSKEIKNLRLGLLNVFKEEAGSFPETERIANNLILFLKRSKSGKLREGEKREAWKKELEENFQL |
| | | KQGILDQWPDTGNLSFCLHPALEPDFERKTERRELIEKRLQELVRLARGTRCEVRLSEQRKSAAKKRGERSEE |
| | | KRRAKVEQGKQQQQRKAIDFLGLDVECNGLNNFFGEVFEKLRSERGLKQLKEQILVLPLWQGKEDELERRLR |
| | | FLAASARSLPASKLTGKWSDYRGTLGGKIKSWWSNYMGFKLKLAKLLPAEEAASACLVKLLSSSREEEGDLE |
| | | ELRRNTLVCLRKNLGYLEEITASGGGAEALVKLYRKYGYLRGEIRRNLIRLIYQGKIFETEREKLIKKAGGGPIS |
| | | PRKCLPSQARKILDNLPRLPRFLGEAAGEEVKLMQEGPRALKIAYELALDADRQLREGSFCPPARDRYLHVLN |
| | | SLLSLYHRLNSNILTATVERIIGEATRKKEVPPSGRLFYVSPKAPLHVRERLVDAPGISADGSKELAKQWERLIT |
| | | LTSPSSPERFLQHTTEQQMDEVELLKIRLAFLLHCYPKTSIAVERWRDLACRVPQTSASLLSALESPSESRLRRE |
| | | ERSVVLNELGSVLRGIVTRKSRRAITLRSTIQLMGAEKKLQLIAAPVNPRDARWKRWERKFAREKSLSGQALS |
| | | RFYRIPHRFGYYLKVGAGFRGRDGRGDNKREAGAQVKCLAFKEKGNEAELRSIPAARIAWIRTSREQLQFLE |
| | | WLLVKPRRKRAILLKRREPMLVVERDLVLKWKREKLEAEAGTSPKLFINQPFQIIPQFEDENVLRLPYRRFLGV |
| | | DIGEYGVGWCVLEEKGEKLRIVRRGFLYEPLVREVREYVAAWRESQVFGLFASPETKLARLRENAAHKIRNR |
| | | LHDIALRYHAQPVYEYQVSAFEAGGDRVKRLYDSIKRADKIFDSRAPRALKSERVSVWGDNSKWGEEEVIRP |
| | | GGAEVSARATSRTCTSCWRSVLDEGLVPREEKFRVEWLGQGVIRLNKEGSGRRQVSFFGFLSGKYKGVTEVIG |
| | | QEARALAEKFSRPPLPEGKKGVPRLSVALRFAFRERRRRGFSVPSLEQLAQFYGKRGSRALFICPFCLHIADAD |
| | | QQAALLVALTRWYSLEYGGGRLSGKLGRKAQGAKYFDLLDLLRRIEGSSPLRKKIELLTVSFP |
| 105 | 2237917 | MANRQISPVDSTNNFIFTLYVSFDTVLYARILAFLTLSCLLVGFLEYIYPLGVIYLIMPTNQQYDGLTGYQLHTE |
| | | RLRHTGKSGIRTFKLPLKTNDQELFGNFFENIKHDHEGQIGATNITTWTEEQKSSRQIYSLLDFWLDSVRAGVV |
| | | FASSSAELNELLSVCGDEDAVDEQVKTAMRAINPTFFKHLKFNDFKEEIALREGMRSSSTENSVTRRLYKCLD |
| | | CTEEDAPEEIQQAVADLIKTFFTTDGKIISRNDQDDYWQVHFGLDKSLYKLDSDIKLTFSFLPDIPFTPEADAHM |
| | | CLDKYRCWIDENAEKFQLESKEDKTNFLQIHWGIEGNYNAFSNYFNQIIDLLREEEGRQKLLDALLNTSDLWE |
| | | GAETELQKRFDFLVDKAKQLPKAKCVDSWSDYRTTFGGRLSSWLSNTLNQEEFIKETVSDQKKELKQIIEKNL |
| | | YKKAFSAADDNRAVELSHEAVIAQKTLEKLQKSSSFEPQLLGVYRDNLGRLRSLLNKAHQLLPDLNKAHQLL |
| | | PDEIENKSAHEVYSALHERIRLMPKFIGGAKAARFEKYVKSLQILKQGASFLENFEEKVKEAMKSSVSVEVEEI |
| | | SEGYFLRQLNTLKRFYDNANDSRFKGKLSSLFDKDLGVNIEAVSNRETFYISPYSKHDNRAVISVEVANYQQL |
| | | LKSWVNELKPYWGNIIATENWGEIIDAMQLERIRIGWIYKLYPKLTFAISDDLDELFAKAATYRDLHGHEVEGS |
| | | LAANFLQRIILSEIEGRVKEASREKFTVRYVIQPVGSEKTTKYPLFVTSNKDKLSATERKQWYIRDINSSFSRSET |
| | | DKKGWILKNGKDFSNGFQEKYISDNTDLFKIISSKYQLQFLDKALPNGAKKFGKDGDVNLKLSEHSFIAEETLQ |
| | | ASWDFSTDTVSLKSVDKDLFVAIPVNIEPESQRSSREERTRYLGIDVGEYGLAYTLIETIPKVRVIKSGFLYEKTL |
| | | RNIRKQVTNIKNSQRLGTFGVPSTKLARLRTQAITKLRNKVHDLVVRYDAIPVYEWQISNFETKSGRVTKIYNS |
| | | VKRADTNSGANENADGDEAALVWGKRPYKIGQEVSARATSNMCSNCFKSIYQFTEQDFTEESRSGEYVEFKI |
| | | AKNTVKGFVPNDEKITSKQKMINAVHDYARPSVDILVKRNDVGVDKEILVNFLEHRGASALYECPFCSHLSDA |
| | | DVQASLFIGLRAICRDQDDETTKVKISDLMDFAQKQNIDPISADFIQ |
| 106 | 2237394 | VELIVNSFFNNDGQLILDGESQFDFWEKEYNLDKGIIESAKPKGKYTDITFVIIPELISNLNTKQTLEDLINKRAT |
| | | WLSNRELDEDNKLLASILGFSDNANGFSNYLGVVLRGLQEEDGDKEALYEAQKTIFPRLENYRDKVLESLDFL |
| | | SQKAKLLGETSLPLVNCWHDYRSVFGGKLQSWFTNFQKRKEELDGQISQFKESLFKAKSYLQKENFGEEADK |
| | | EKEDILSFLSLLGNFFTDEQKSIKIEENYQLFEPLIALIRRRLNFFYQKYIQKEGDETKVNKFKEFEGLYKTIYKP |
| | | VAFYGYSAKRANEKFVNKTVPILEDGIENIKKIIFYLQDSFSVQKTFEDVKQEREEIDDPYRKFLQFFWNKYLD |
| | | ASINSYLFLGKYEEIIKENVEDNKWKEISDKNKKGRYVFYKSPYTKGTLEKIPIKANSYLDKLQVSVLELVEFIL |
| | | SFKKDTLLSDSALLLDWVELSKNIISLLLKENTKETYKLDGLLLENFSQAKKYQELFKREDYPKNEFSFIIQSLV |
| | | LSEIRGAATLYSKREYIASYSVQVVNSDEKFKLYYIPKEEIPITPDTIKSKPDSIERKQLMKPHCFVVALDKVSEE |
| | | KKNQNFNAILLSKSNTTAVFLPDESLQKLCKLSSSPYQLQFLDKYLFRPAGWENIDISLGEWNFVVEKGYKIN |
| | | WDLQTKKPKLLPITDSEQAKKNKIYLAIPFNLSPNNQIKQSAPLKSIAKGNEKKEKDLSRLNYPILGVDVGEYG |
| | | LAYCLVKFDFDKNTYKITGIKLGSDEKETFGFIEDKNIGNIKDKFSEIQQKARQGAFDEEDTIVARVRENAIGHL |
| | | RNRIHIVVTQEKSSSVYEDSITNFETGSGRTTKIYNSVKRSDTESGTKADEQIHNHIWGEKTKLVGRNVSAYAS |
| | | SYICVNCLKSLYQIKKEDLTKMTITQKDGRSRVVTISSPYGDIKGYVSKEEKYDVGYQFRGNDEDLKKLRKIV |
| | | QDFARPPVSNRSEVLEKYAKPILETGRIEEWRKRRGNSSIFVCPFCQYVADADIQAAFMMAVRGYLRFSGIVPS |
| | | LSNKNKQDQEKEKTAGESFLEQTQKQLKNIDINKIKSAFSIRI |
| 107 | 2237771 | MLEVLRKDVEHDSRHLYGASNLSDYQNKTVTYSVIDFWLDAIKAGGLFQPSAIKLKNFVSEENIKADSKLIAF |
| | | MDQENLSTNLKSLPRAQTLENKDRLAKKVAMWFIEEFREKDFVLSAINEIVAGLTSSTGNQTKQKEFLLSYLG |
| | | LNKDFFDKETTLTYFLHPDIPNLIDKGITVNELLEQRYKFLQTKTGENEVDIKKLQELLGLSDNFNALSHFLGKL |
| | | WMTLKNEGVEEIYNQMLGISNLWQDQEDQLKTRLNALSTKAQLLPYPKMANHWGDYRSSLGGKLQSWSTN |
| | | YFDYTQKSLQKLTAYKKDINLIISSLEKQKDNTEYMMLLKTAKESALMLNNLSDVPDKDRLNVLSATLAELRI |
| | | GVNEVFQKSPSVFGYTKDEELLTFDQAFPVLTKPTPKYPEFFGDSKVNKFKSFERAKQTIENGVAVYNNLDVI |
| | | MAKPNNLDETRILKFLDGWITYHSGCNIETIKSLVAELITSYSREPLENNRCRFYVNPNNPRNKLLKTVDVGKP |
| | | DLSILLQKMVVLKENINRHIPESVTNNNCDYLIDWVEFCKRYYSFLLVGSTQTIDALSLCTDKFPQAEAYLKLN |
| | | GTNLLPAPAVAKLINRFVSPEIKGAINLKSRKNITERNTLQMVGALDKWRLIVKLVDIKSKIANLPPQISTELQN |
| | | LKTTDDVIQAKQRSIRLAKFLPHHFMLYVATRTPVPQETHALVYHKSSGSYRPGAISGDSLFKIASTPEQLQFLE |
| | | MTLGLPKRYTNPQTQQDLLNIRIEEPSLITEHEYTDQTQAIHKPRVFVSVPFKIWPVYKNNVGHKTNKLGVDLG |
| | | EYGVAYSVITPKEQGGKMDFVCLDKGFLYEPQFRAISAEIAETKARQLRGTFSMASSALADLRESAHKSIKNRL |
| | | HFLATKHNAQLVYERQVDAFETAGNRVKKLYRTIKRGDVYPKQLTDEPIFKQYWGSYDKTNPPATEIGAYGT |
| | | SYFCPKCRKSIYQLDKTIVVVELEKQNGYPGVYLVHTEPKDISFYIYCEKDLPNETRGLIRCGKLFARPPLESVA |
| | | VDLCRHKGSLDRQFSSKALANRGSQPLFVCPFCLSCYDADIQASINIAIKRVLFENSGKNKDFNFYKESTADKV |
| | | KNYPEAKLD |
| 108 | 2237796 | MAQQIRRSTYATGSLAHAQRVAYSGGNESVKTIKFPTEMQLKADSPEILNGIAEAIMQDDIAILGARTEYSHYY |
| | | LAQQGKEVYSALDFWIDAVRLGMVFAQSAASLLQTVGVLSNAQTPIALMLGQSSVKELLNMHGAESFLAKR |
| | | TFGKGSSVSGVSKNIIKCFVQGHITEEAAEAKAKKLLALGPAEQKQYKKQLLGIAGIAQPEGYRNTLGITACLL |
| | | PGFETPDAYRAGRDVTLLYHVQRMQRLLSLDEVKEAYEFVGMHDSALSNFLNGNSNKGFLALLLRGEFDTLA |
| | | RGMMDMTPLWNEHNHDVLMNRLQALGRNAQKLSFTKPRFGNSFADHRKQISGKATAWFSGYCNKLDIAKE |
| | | QIPLVLEDAKMFMEMLCAVEIDYEKEEFMHLQLSAFIERMERAHERLDADGVVALKKAQYVLPIIREFANPIV |
| | | QREEAQQWLSLNIELVPDKRFTFKQAFPYLSDQGGADTEKGEQTKFQSVPRFFAEGAHAEYVAFSQAPMIFSV |
| | | YMENLRLLWDRLIGMERRTPRNWEEHLSKLLYYLYVDIYVECRTDACKKYVRGILETYAHVSRIPEHVPGIKR |
| | | FTIKPGYTPQGGLTKDQLVQCVLQIAQRLSNAGFEWKRISALERLDLVEVHKKAFAFLVGITHGSVDVSAYNW |
| | | LNNKSVVQYLDVLKTTDLGGIRLARFLQSCVCATLRGSATRMSEQMLTARFSVQTATTIPQCELVYAVSEYM |
| | | RKRRFVTPPSHVSRDVLDRKPASIVAQAIYNPEQMAGSMRFIPHRFGYQIQLPELARHESLNNALVVQKPTSTR |
| | | RYSFARFDAEKGPVLWVESSHYQQQYFDWFFFAPNNRPADVIPQGVSLVVEQDIALQWDFDALVVHMQPVH |
| | | EPRMYCPQPFKFVPRVESSVMQNRYMGIAPGTNEVAYAVIEVNGTRVSVINCGTFPLAGFANLSRAEEKKQKR |
| | | ERRVGSHAFSPDKRAIVLDNSANKLANQIHACAIQNRARLVWQWTPQTVQIAKGREVDMVYARARKSCAPK |
| | | NDSDFDAMKTKWGKIWSAKWEDGKHTLSTEVYYAESLFQSACNAPVPPECATALMV |
| 109 | 2237901 | MAGFGPAICLIQFLCSPVYPKTNIIFSMPEEKDRPKKYKGLTGYKLHLKRYEYAGGGSTRTIKFPINCENNEYRQ |
| | | ELIKKVKHDYKRLFGGMNITTWIQNPSCYSLLDFWLDTLRAGFIFGNTARELQKNIYYILYGKDVTEVEIKPKM |
| | | VAKKPDFVSWLDWKSFCQEFVQKEGVRSRDGVTIETIKKKIKKCLKKEYQQTEDQQTSLELEKIAKGIFEKSIA |
| | | EQEKFWRENYGLERLKKTGEITIFPLPDLDFDKNSKVETLIIKYIKLGKKMRVSVENLIGVGNNASGLSNYLNDI |
| | | FNKLLNNKNEEIINNINSLNSAWKNKQTDLDLRLQFLSNKAKLLGKPKLVNNWSDYRTDIGGKIQSWLSNRD |
| | | NQIKDIGEKLERQKAGLEKLKNEKVIGKIASNCLEYVTNSLGSNEIQTEDKLTIYREMVSDLRERVNFWQQEN |
| | | REVGEKYKTEIEAIKEKIKLVPLFVGKAKAEKYNNYLNCQNLINEGLAFLSEIEKITSKNNQVILTTEEKKKEIN |
| | | KLFSKYHQLNTSRFKGIIKKLFNRIGVDDISQYDPKKETFWENKHAKREKKIIEINSEKLNNFDIFQIAKDIAPRW |
| | | EPKTMIDEEMIDAIEIEKIRWGWLTKTGKVKLIDKNLVYLTRARELIKSVAKVEPGLLIQRFVLAEIKGAISQKS |
| | | RKFFTVRYVINPGKFNEKFPLVYVPKNGKKISRTNAKYWGYVKKEDISCSKNSEKIAYYWQDKSNRPKEGRL |
| | | TTTDSLLKIQTSKYQLRFLEDVFCDNRSKTWIKHAPTISSPALILEETVKVKWANNQVKLEIAKQQLYMSQPIV |
| | | FGYVDNKTRERLENQTRVLGIDVGEYGLAYSIIEKRESKIKLIKKGFIYEPALRLIRDNIAKNKLSQIKGTFSMPN |
| | | TKLQRMREQAISGLRNKVHDLVVRYKARPVYEFQISAFETGSNKTAKIYNSVKRSDVSESKPNNIDKTIKNTIW |
| | | GSTFVKVGSSVDARGTSRLCSSCKKSFYLYLHDDKKSKTKYKVENSQIKLDNQNWNIYNKTGANVLNSKEAS |
| | | TGLYDYMRPPMKTFLKRQERSTIKYTDKELEYFQKRRGDSAIFTCPKCGHVSDADIQASLIIGLKEFFNREYSS |
| | | QKQNFEKFMKYVSGLEIPTVGLEVD |
| 110 | 2237118 | VKAITGYRLHKERLLLSGGGVVRTIKYPIVPLNHASQKSKEFLKQFERGIIEDDLKIRGSLNLNDYLRDKDCYT |
| | | LFDFWEDSLRAGVLWMPKVNDLIDFFNQEHIDHIWQNASSKITYYFEKDKFKEILKRIPVRKTKKRANFLKDL |
| | | LSSVKKDFLQKGKDGEYEVINQEVNTLIGSIVNTFYDKDGRLVLEGEKNQSKFWKEEYGLDVYYLKQIQPAG |
| | | DYQKITFVIVPELIELEKNSSLENLLDKRREWLLSCFKKEEVKKEELEKILGLSDNFNAFSNYFGVVLQGLQNG |
| | | KTTEIFEAHKTIYPEVEKDSDKIKKALDFLAEKAKKIGKTKLSGVNGWHEYRSIFGGKLQSWFSNYQRRKNEL |
| | | GEQIDKLKIGLDKTKNYLEDTKFDSDEESRQKKEHILSLLARLKEVTTEEGGKSIENEKDYLLENDLLAQVRSN |
| | | LKFFYEKYIKEEGEEKPVNQFGEFKYIYQTIHKPVAFYGDVAKTKNEKIINKTFPILKDGIENIKKIVIYLKESFFP |
| | | EDFLDQKSSQETYKKSLQFFWNKYKENALNSLEFRNYYREILKEATADDQWQELQKKENKGRYVYYKSPYA |
| | | KGTLREIKTKEVKDFSERVTELYFRLAEKVLSYEKNNLLKDEKILLDWLEIAKHIVAKLLDYNTKERFHLKNLS |
| | | VDNLEKLKRYVDLFRGDGYSKSEYGFITQNLIFSEIRGAAALFSKKEYTAKYSVQVVSSDNKFKIFYFKKGENE |
| | | IDKSYLEERKKLMLPHHYAVALREVKTKKKGGGNSIVLEKNGFYPVFVNHDSKDYLFRLSSSPYQLQFLDKFL |
| | | YHPKGWEDIDIALSEWSFVLEKRFSIKWDLQEEKPKFNPINEGKKAQKNKLYIAIPFNLTPSKQAQKNAPLKIIA |
| | | KKGNKRDLSRIDYPILGVDVGEYGLAYCLIKIIYEQKPQNVKAIEILKDEKEKPVCGFIEDRNIANIKDKFSEIQK |
| | | RSKLGSFDEADTVVSRVRENAVGNLRNKVHAVITINKGLSLVYEDSISNFETGSGRTTKIYNSVKRADTKFETE |
| | | ADKAEHSHVWGKKTAIIGRDLSAYASSYTCIKCSHSLYEIKKSDLPSMKVIDVDGRILAITTPYGNKTTIRAYTS |
| | | EKEKYKTGYQFCPNDEEFKIFRKMVRDFARPPLSRNSEVLLKFASGLTEKNKIEMFKKSRGNSSVFLCPFCGFI |
| | | ADADIQAAFFMALRGYLRFSNTVSPSGKENNEKTTWENFLEKTQKILQKVEGKEITKYISLRF |
| 111 | 2236779 | MGNSRKYRGKTGYTLHAKRIEYSGQSKALRTIKFPLITNSGVLFSALHDRVQHDHLYLTGSTNIDDWINHGEIY |
| | | SLVDFWVDALRLGGFFQPDAKSMGRLLDLMEGRNTIYIQMVETMPTSLRRQIDTHQLINDLLKTTGFRKSSSP |
| | | ERIFARVVQAFKEEYRESQIARQTASIISKGVFEKDYGEEQVRFIAQAFKMKYIADDRSLTFFLNPYLEVDQESS |
| | | PALLVRMLEDYFTPYIEDGITNLDDLLGYGNEANAYGNFLGQVVPLLQAGKVGEIVDAFGKHSALWLGRERE |
| | | LRQRLQYLAERAQRFHKPLLVNSWADYRSVFGGKLQSWYSNYKRHMLQVRERLELQKPEFERVEFEINKAL |
| | | RENDDRYLRETKAAVEELGKILEALLEKYQIGGETIYEDDLMMYRDLLGQLRVRINSLWQRRRIFKDKKDGSE |
| | | SKPIHKQLRAIFRNMRVVPSFFGRSKFERNRRFLDSVRRLEEGFAILAVLRDRMLAKPLDWEGVDDKYLCRQL |
| | | DTMARMYRRGGTNLFTSKLTKILVDYGGKVPGERERFYVSPYSRERFHLVAPAGRKKLVPEALVDWWEELK |
| | | FKGNWKSITDKQLVDLLEIEKVRMGWLLRLHPDDEWKLDDLRPKYFEKARSLIEINNGRLSSWQFGQFVQAFI |
| | | LGEMRGTASLMTRRRFAVHHVVQPLESEKKFVLQAVFKNGETNEFGPGEQYVFAVTVPRLETGVPAEESGLT |
| | | SDYVVPIREKEVSNKEALCRNGGPKKLTDTFRIGGSRYQRQFLEWFVMPHRKKGRVSLKRSEPMVVAETDYL |
| | | SVWDKTSGGVKLTPRARRLFYNQPFAFTPRGLDTMIDHLPGRYLGVDIGEYGIAFAVIQVQEEDGKVELIDRG |
| | | FFFDRAVRKIRDRVNELRKRQMTGTFAIPDTAVARVRENAIHTLRNRIHDLVLRYKAKPIYEFEVSAFEVGSGR |
| | | VRKIYHSVKRSDVPSDIKADGLERKAIWGNQYKDGLVGNHIGAYATSQTCSRCRRSVLDWVDREVNYRVEID |
| | | PRTRRGKVWLSDGVKIEIFVDDSVDPDEVTKISGKEVNKYAYKFMRPPKDSPFLENRGVAVSEAFKQHRGNQ |
| | | AIFVCPFFDCHHISDADIQAAVHIAFKGYLKDKEKRNKTRYDFFDELGNLSLYPEVGFDDSLF |
| 112 | 2235858 | MEKSKRITGYHLHIARSLYSTKTIIRSIKFPLKSPSHDAGILFAENIRNDYLKQVGNIHPSAYFASTIEYSLLDLYV |
| | | EFLRQGFLFAPTATKLKSKVYKITYGKSLAEVDIFPNISQEFLNLVSKDLFDKTILQNSTVRNTSTKASLDKRYT |
| | | SIFKKEVLRETVLEQVTKMVDGVYRKNYEEQKQFWKNFINLEFSEKESNLTCYPLPNKGDKFSGKTPIQEIILS |
| | | HFESKKKDLFGTQRQDLTDEELKLARIELLEDIGLSANFNALSNFLGSIVPLLLTEKGIRELISKILNYSTVWKDK |
| | | EEILEGKIAILALTARKISLPSLTASWADYRSSFGGKIQSWVSNFLTQNVKLREALNEQNKEIQELKKEYTIDSSR |
| | | VIEINRQLLGNLTNQSQYQLYVDSLAYLNTEINMLRQERDVDFKLFQDKYKALSSNLPKIPNFIVQRKFTLFQD |
| | | FQNSLVKLEQGFSYIHSVYNELLGDIRVSQNPDIDGVLVRKTLDSLKRFYCYLEEGSYYKNVFKEFFLSIGHTV |
| | | SEIEGTDKRFFIAPQSRDKQLSLIIVSESTFEIIKDKLLKIPIDFSWKHQSISELLAITEVMKIITAFYLKMIPNTKEF |
| | | TLDENTILHFPAGQRYLKTLGKSLNIKHFSFFLQRYIFAELRGCLNLLSRETYIERYVVQTLGSDKSFPLVLFPKD |
| | | KEQKPSITNRAYWGIVSQRPSRNQKDQDDTLNGYIFKSKEYNQGAGSFKENTKIFPNEAVFQIRSSKYQLQFLD |
| | | NPLNDLGDRSLWKRVSVKLSEHSFIVEDTMRVTWDREKMRPNISCIKTDIYVSIPINISPNIKPHAVNPSRYMGI |
| | | DVGEYGLAYTIFETNPKFKILKQGFLYNANLRKIKSHIKRGKEKQVRGTFSSPSTSIQLIREQAITSIRNTVHDLVI |
| | | RYKAKPVYEWQISNFETGSGKISKIYNSIKTSDTNSDTDAEKGAKELVWGKGTKTIGKEINAYATSTICSKCFK |
| | | SYLLEIGLKDSDKFTLEKIGNETFIKINNTLIRYFSRKPITNTDDAKSAIYGTMRPPQYSEIVKSLDIEDRFFSTRG |
| | | NSAIFYCPFCKHISDADIQASLFIGLKGYLKDLEKKTETTKNNTQVDRKTKSLDETRRMIKEARLLVKDPILFDR |
| | | NNFLSFGEHLVNT |
| 113 | 2237006 | LILEGKKQDNFWLNEFGIDKSIIQKTKPEGELKDITFVIIPELIVDSFNKEYEVDSLIEKRRVWLKKRFNKEKEIE |
| | | KNLQLILGLSNNFNGFSNFLGKGLRAFQGGKILMIFEAMSKINPSIKNQENKEKVLEALNFLSDKSKFFPLRPSL |
| | | NIVKSWADYRTFFGGKLQSWYSNGIRRKNELKVQVKEIYEFLTKAQDYLAAKISENDENKRYARKELESINLK |
| | | INRLKQFIENENFDISSEDRYLIFDTLLSSLRTQLNLYYQKYLSSEEDNIRENKDLKGIYQKIYKPIAFFGKATKR |
| | | KNKKVIEETVPIIESGINNLFSLMKKLEKTFLPKNTFSKVKNKNEDEETNLRNLVDYYQNKVSHKMLNSLTFV |
| | | NKLEEVMKSVIEENDWDKLHSNKYVFYKSEYQKGALELIPLKKGSWIDIFEKIILEMTSYLLTFDLTDLLKDKK |
| | | ILLDWIEIAKNTLAKLIKFNTCDVFTLDELNLDLRNFPKAMDYIKIFKITKVDKNELNFIVQSFILSELKGAATLF |
| | | SKEKYLAKYNVQVINADKKFKLFYKPNDGFIEREVDRKNLLKPHQYFVALDKIEDKKIKEKANFLLITKEDIKP |
| | | VFIKEENFSKLYKISSSFYQIQFLDKFIYMPEEFKDLGIRLSEWNFVLEREYKIDWDLCTKRPKMTFIENSKKNK |
| | | LYLSIPFNVFYKSKKKDVSLSKVISNRLSYPILGIDVGEYGLAYLLAEFSDKKIKILKKGFIEDRNIANIKDKFAEI |
| | | QLKARTGIFNEEDTTIARVRENAIGNLRNKVHYILTSDRGASIIYERSISNFETGSGRTTKIYDSVKRADTEFETE |
| | | ADKFIHNHVWGKNTKYVGRSLSAYGSSYICSKCHRSIYQFKKEDLREIKLLMREGNILTFLTPIGKVWGYSKD |
| | | EKFKKDYQFKPTEKDFKEFIKILKDFARPPVGKNKTEVLEKFFLKDNDKKAKIDEFRKKRGASAIFICPFCGFIA |
| | | DADIQAAFVMAVRGYIRFKESQIKEENKSLILEKTINYLKEVQFKPEDIFLPF |
| 114 | 2237276 | MSKNLTGYRLHLKRILFSGGEITRTIKFPLLTHNDDVFIKNFKKNLIDDDLKIRGDLNLNDYLKYTALGNPIYTI |
| | | FDFWTDSLRAGVIWADKPSSLIDFIGDYYQNEKSPCNLVWEKANEEIKEYFDKVKFKEILISDPLRSTSQKDSFK |
| | | NRLIKCLKNEHFEKKEKNFLVKSLAAKDIIERIANAFFDDKGNLILEGKEQNKFWLDNFGIDKSIIEKAKPKGEL |
| | | ADITFTVIPELIEDLEKECDVEYLVEKRRFWLKKRFNDTKEVEEKLEKILCLSNNFNGFSNFFGKALRLFQKGEI |
| | | EIIFRAITKIYPQLESPENKKRVFKCLSFLSEKSKLIYKEPSLNMVKNWADYRSFFGGKLQSWYGNTLRRKKEL |
| | | DDQIKNFKNSLNIAKKYLFDTNLNQEVDEDQLKIIRLKIERIEEFLNKYNIVSEKNYSLFYEILSSLKSHLNLYYQ |
| | | KYILKGDEEKSIEKDDHFSGIYEKIYKPIAFFGKASREKNKKIVESTIPIIEDGINNILLLINYLKPSFSPRDTFEKV |
| | | KNKDETEEIILRKLLDYYWNKRKNKIINSIDFLNKIEEIIRENIDEKKWQNLQIKENWNKYVFYKSRYQKGLVN |
| | | QIEIKTSNFLEVFEKTVLGLTSYLLNFNLEQLLGDKKLLLDWVETAKNILSKLIRFNNKESFVFDTLGINLKNFP |
| | | KARDYIEAFNIKEVSKNELGFIIQSLIFSEIRGAATLFSKEKYLAKYNVQVIGPEEKFKLFYQPKDNLIEDDRKKL |
| | | MKPHQYLIALDEIKEKNLKDKFNFLKLLKNEIKPVFIDEKDFSKLYKISSSVYQIQFLDKFIYLPEKWKDIKVSLS |
| | | EWNFIVEEEYQPKWNLRDKRPEFKLIEDSKRNKLYVAIPFKISFKGKKDNVVIKRVIDKRLEYPILGIDVGEYGL |
| | | AYALVKFFEDKIEILDKGFIEDKNIAKIKDRFREIQQRAKSGIFNEVDTVVSRVRENAVGNLRNKVHFVLTDKE |
| | | GASIVYEYSISNFESGSGRTTKIYDSVKRADTEFESEADKQIHAHIWGKNSKYIGRNLSAYGSSY |
| 115 | 2237023 | MSKKVSLSGYRLHVKRLHFAADTPTMRTFKFPLVRKCGKKADFTDVPQELIEAIKHDDLYIRGPSNLVDYLRF |
| | | RNKKIYTLIDFWIDSLRAGVIFMPSTRALADFVTRIKEGKTIFEKIVEEADLRFKQYFNVSKFINEFVAKKEMRK |
| | | GKSRDSFVNTLAKILREDLSEREKIGTERFIAELADVFFDARELKILGFNAQNEVWQARFGLNKEAVDLDFDLT |
| | | FFIFPELASKQKVFITPENALEIFEQLIQKRISWFVAHGYSKEKAEREVDRVIGLSDNFNAFSNYFGEVLSILKNN |
| | | RVEDVFKLLTKLYPKIGEIEKRQLAWLAERLQGLEYPLMPGTNGWHEYRTLIGGKLQSWWSNHKNRRQEQM |
| | | EQIERLKRELPNLQEFVKHYPENELEIDEEVMISRKIEIEELVEKISEVIKQPDNIFTDLYRYEFIETLIRQLRQRLN |
| | | EYFQLYAKGDDDKSPEDNKYLKEFWKIKIYKPRSFYGYAQRERLKKIVETTIPTIKQGIKLTQHLLLTLPSFVEY |
| | | ERASKEKEGGKKEARSDIGLRRFLEALRRKIKDDKLNTKIFKDLYINILQPYLPNEDISLLTDKRKLNRYTFYKS |
| | | SYSTSTQKELELPAIDYSKEAENIIKEIVEFLVELKEEKFLKEPGLLLDFIETSKVVIARMFRWMDQEEEWHLHQ |
| | | FKFDIFPAAQKFVKELKEEKVKTPMLRFIIMSQVFSEFRGAATVFSKKYVTAKWNVQVVGSDKKFKLAITPKD |
| | | KHIDVNNLHKVLAVNDPEVLKNIKWNVVISSKVGNLQRMITLKNKNGEFGITKEKIDEGNLLQICTSKHHLQF |
| | | LERLIYKPQNWKNIKIKVSEPSLIVEERYQLTWDLKTALPITTSHNVNKSRLYYALPLQFEVERSKRSQLAEIKF |
| | | HFEVNPSLALKKYYFLGADIGEYGIAWVVLQVDEGTREIIVKDWGFEYDKQIKKIQDEFREIQQKARLGVFLE |
| | | DTTLLAKLRENAIGYLRNKLHDRLIDYGGHIVYEANISGFEVGVGRITKIYDSVKRADVGNHPDMGRDVGKK |
| | | LVQHIWGLKIGGKKNSPGWQVGAAGTSYTCICCGQSIYQLGNNKKNKLKEDKDKKDK |
| 116 | 2237289 | SQLIDERKKYFSEKDLEEILGLSDNFNAFSHYFNKFFLLLYQDKQEKIFECYQKIFSFSGEDRKRIKDALDFLSEK |
| | | SKLLGLPKIVNSWSDYRSVFGGKIKSWFSNYLNREDKAKKQEKKIKEGLEKVNKFLLDFIQKNQVDSDLQQEI |
| | | KFYYDKLNQFINSYQNQEFFHQQELFLLFSDLLAEYREKLNRFYQKYLSDKEKEEKKVDEFPLFKDLFEKYEG |
| | | PISFYGKTKLEDNKKIIDLTFKTIKVGLNLIRRLLLDLYNSFDFKNSDNNNQERDLRRIFELLLNKIPATKTFREK |
| | | YLSILKDNFDQKTYKDITLKPSQYTFEENIYSRENRKLIELPSKNFEELLSKIIKDLTDFSLSFKNDDLFADIYLLS |
| | | DLVELAKTLISLVINYSNKSQFDSYKNELIDDTYQKAKKYLETFKISFFNSKKEANYFYQTRVLSELKGAVPLFS |
| | | KKYYQAKYNIQILKSNEIFPLFVKFSDLLKKEEINDINKLKLIFKKPYRYLIALKKIKFKKNQQQSSVIHLDKKN |
| | | KDLVLISPQDEDFLFKLTSSFYQLQFLDRFVYPVKKWLDVDITLSEWSFILEKKYKINWDFNNGKPEFSEIDSRL |
| | | YLNIPFKIKAINQQKILKPKELFLGIDVGEYGVGYALVNFKDEEIKIIKSGFIRSKNIASIRDKYRLLQDRSKKGV |
| | | YFSSTNVVQEVRENAIGEIRNQIHDILIKNNADLIYEYNISNFETGSGRITKIYDSIKKSDVYAENEADKSVIQHV |
| | | WGIKKSIASHLSAYGSSYTCSNCGRSIFSFSENDICSSKVIKRDGNIITIQTPKGEVFAYSKDKKFNVGYSFSQEK |
| | | SKEEMKNLFMKIVKAYARPPLLKSEVFLAQKKLDKEFLEKFKKERGNSAIFVCPFVDCQSLADADIQAAFIMA |
| | | LRGYLKKKKGKDINYLEESLNYLQNFKGKINFSNLLH |
| 117 | 2237415 | EPDLDPIACFDRLKNIMSVEEWKDVYSLLGIDDNCNALSNYFREVLEDFQQENTQSLKKKFKNVIHVWNEDQS |
| | | GLEERLHWLEKQAQKLSKSSLAKSWADYRSNFGGKLKSWLSNYHRQKQLIEDGLKNQKEEFQKIDTQKDSP |
| | | DFQQEGSVKIAQLSKQLLKIINETLNKEINLTYQRFFLYRDLLAELRTQLNYYHQAQDKSEELVREVYPHIFKD |
| | | LELVPQFFGKAKTQRYQKYIKTAELIEQGFNFLNNNFTSFSTNQKQIETDVLRKKLESLHRYYFTANTNRYKPL |
| | | IETILTKYTETIPPENHKYFKHPRSTDPRTKQIELNLENTNTINDLERIQKNLNLESDWKHLSAAQLIDYIELLKIK |
| | | WSIAVLLSDQKILVKPDELSLFDKASAYLSIFGNSIPSEKISQFIQSFVLSELKGAVNLLSRKEFITRSTSQFIESHE |
| | | KFPIVYLPLDKKFGKDHIPEELRTEFPKTAEELLGDKSKIPDFKILSYYPHKWAVIDRDHIKNTPVENYIPALVIQ |
| | | GKKRDEFKVKYVDKNELLQIRTSTYQLQFLEKFLRPPAKWRDIQLRISEPSLILEEKFKVSWDRQNLQVKLDPA |
| | | GSRQLFYSLPFNLEPQIDTAARSKAIDNRNAYLGIDVGEYGLAWSCLKFNQESKCEQLGNGFIFSHELREIREH |
| | | VANMKNRQLKGTFGMPSTSLARIRESVIHSLRNKLHDLALRYNAKLIYEMQISNFETGSARVSKVYRSVKTAD |
| | | VHPENDADRAVMEQTWGKGTKYMGTHLSAYATSQTCSKCHKTIADDLDWESSFEVKRQVEGSIFELQNNQE |
| | | IFKVYSDRKLEIGQQLDEKTAKNLALKFMRPPAESDVLNWKNVSVPEEFTATRGNSAIFVCPFCDHVADADIQ |
| | | AATNIAFRGYLRDKYKEEKSDERFDYIGKIKKHAEKNEYPLNELRV |
| 118 | 2237512 | MPTGYSLQTKRLLYSGGKATRSFKFPLVPFSQKSEAAYKDFIGEVRQDAVNMMGALQLFDWKQKGETYSLA |
| | | DFYLDSLRAGVIFAHKFDAMKSVVLALGGGKDPQAFFAEQLRAHHPDLADLVDMGAFYNFCTSNSRQRNKT |
| | | KEELLKILAKSLFTKTSWKDQSDRVAIYVGQITEWVYDKTNAQKSDAAKEFLGFNPKPESNFDFTAVISLDLPF |
| | | VPDMSAYNLVERIGAYIEGQAAYTQYKAEDRLSAVLGLYNKQGAFSNFFNEILSDLKKEKGHTWFTAMTQAL |
| | | PAWQKREPELRERITFLAERAKQLTAPALARGWHDYRTSLGGKIESWASNFVRQKDEIRKQLFGHETITERGH |
| | | DKRKKIWRNGHKQTLENILEDDIVKKHSDAVALACAELKCIATMELHITDALLSLYSDQLGQLRIALNEAYQA |
| | | EYPENLEKNGKERKQWESGHRANRKYAQIYDDIKLIPRFPGEAKYEKYRKLVLAPKLVKTAVAYLQQSSEML |
| | | RKQSCPEHADEECLQRMLETLRRKYFVLNGTIAAHMIRNALKPFACSVDGKPVTMDDVLNEKQRENNKDYW |
| | | YFWRAKQARARFGRKIDMKYDKNVDAALTKIAVAIAPRWTDRIASGDIGDLLDACELEKIRMGILVDATVGD |
| | | TWPLGLDTLPGEMFEKAHAFKEVIGDRPATRAEFGRFLQSCVLSELKGALNKMSRKEYTERYVVQPIASHEKY |
| | | PLFNHEKQWGIALDKKTEGAIKISQKSKAKGFDDKAFIMEPQREERLLTIRTSKHHLQFLQKTLGGKDSWWHR |
| | | KKMGIENGEYSFIAEVPMRLRWDFGAETLQIEEADKPRLFVSIPFTLTPEQKKEESHLAAGKRKRYMGIDVGEF |
| | | GLAWTVLEMRGDAPHTVGHGFIYERLTRNIRNYVQTLKTRQMRGTFGMPSTKLERLRENAITSLRNQVHAIA |
| | | MKYDTIPVYEAEISAFETGGNKVKAIYDSVKRADTGRGGIEAEKMEADLVWGKWNGKGNAGKQIGAYATSY |
| | | LCTKCLHSLYDKTPEADRSAEKKTTRPSLDDERVRKMGLPETWKNQRGNSAVYVCKCGHVSDADMQASYW |
| | | IALKRALRDKKYGEQTDDSNEKKTVEIDDLRAAHRENPTVITLQSDGSVHYAPSSAL |
| 119 | 2237401 | MTKRKSRLAGYRLHRERILFSGGQVIRTIKFPLAPLNPSEETALFLKDFERTTIADDLKIRGDLNINDYLAYTAE |
| | | GKPLYTLFDFWVDSLRAGVIWMPKGTKLIDFLASQYNTTSPFDRIWEKASPRITSIFKKDKFKGIFLSDPIRNTST |
| | | KNSFYKRILESLKDDLVKENIIISTDAKKEVELIVNSFFNDDGKLILDGESQFNFWEKEYNLDKDIIESAKPKGR |
| | | YADITFVIIPELICDLNTKQSLDNLINKREAWLSDKKLLSSILGLSDNFNGFSNYLGVVLRGLQEEDGDKEALYE |
| | | AQKTIFPRLENYRNKVLESLDFLSQKAKLLGETSLPLVNGWHDYRSAFGGKLQSWFTNFQKRKEELDGQISQF |
| | | KENLFKAKNYLQKENFGEESDKEKEDILSFLSLLENFFTDEQKSIKIEENYQLFEPLLALIKRRLNFFYQKYIQKE |
| | | GDETKVNKFKEFEGLYKTIYKPVAFYGYSAKRANEKFVNKTTPILEDGIENIKRLISYLQDSFSVQKTFEDIKQE |
| | | KEEIDDPYRKFLQFFWNKYVDASINSPLFLEKYEEIIKENIEDNKWKEISDKNKKGRYVFYKSPYAKGTLEKIPI |
| | | KANSYLDKLQVSVLELAEFILSFKKDTLLFDSALLLDWIELSKNIISLLLKFNTKEAYKFDGLLLENFSQAKKYQ |
| | | ELFKREDYPKNEFSFIIQSLVLSETRGAATLYSKKEYIASYSVQVVGADGRYKLYYIPKEEIPITTDTIKSKPESIG |
| | | RRQLMKPHCFAVALNKVLEKKKNQNFNAILLSKSNITAVFLPDESLQKLFKLSSSPYQLQFLDKYLYRPAGWE |
| | | NIDISLGEWSFVVEKKYQINWDLQSKKPKLLPITDSEQAKENKIYLAIPFNLSPNNQIKQSAPLKSIAKGNEKIEK |
| | | DLSRLNYPILGVDVGEYGLAYCLIKFDFDKNTYKITGIKLGSDEKETFGFIEDKNIGNIKDKFSEIQQKARQGAF |
| | | DEEDTIVARVRENAIGHLRNRIHIVVTQEKSSSVYEDSITNFETGSGRTTKIYNSVKRADTESGTDADKQIHNHI |
| | | WGDKTKWVGRNVSAYASSYTCVNCLKSLYQIKKEDLAKMIIIQKDGRVVIISSPYGDMKGYVSKEEKYDVGY |
| | | QFRGTDEDLKKLRKIVQDFARPPVSNRSEVLEKYAKPILETGRVEEWRKRRGNSSIFVCPFCQYVADADIQAAF |
| | | MMAVRGYLRFSGIVPSQSNKKKQDQEEEKTAGESFLEQTQKQLKNIDRSRIKNAFLLKI |
| 120 | 2237018 | MFNQKKGYRLHLERIIYSGGEITRSIKYLLASYSDSQKHKEILNNFSQGLYNDDLKIRGCLNLNDLVNNNQIYN |
| | | LADFWIDSLRAGVIWQSSASSLIDLIKRLNHQETIGEKIFNNANERIKRFFNSEKFIKEIILSDPKRIGSKKQAFYN |
| | | SLFDILKDEFKKQEKNEKIILDNQAEQLIKEIIDAFYSNDGTFIMEGEEKQNNFWQEKFNIDKNIIKKEKEDILKD |
| | | VGDITAFIHPSLLSLKSDVSQLIDERKKYFSEKDLEEILGLSDNFNAFSHYFNKFFLLLYQDKQEKIFECYQKIFS |
| | | FSQEDRKRIKDALDFLSEKSKLLGLPKIVNSWSDYRSVFGGKIKSWFSNYLNREDKAKKQEKKIKEGLDKVNK |
| | | FLLNFIQKNQIDPDLQQEIKFYHDKLNQFINGYQNQEFFHQQEIFLLFSDLLAEYKTRLNFFYQKYLSDKEKEEK |
| | | KVDEFPLFKDLFEKYEGPISFYGKTKLEDNKKIIDLTFKTIKVGLNLIRRLLLDLYNSFDFKNSDNNNQERDLRR |
| | | IFEFLLNKIPATKTFREKYLSILKDNFDQQTYKEMTLKPSRYTFVENIYSRENRKLIELPSKNFEESLSKIIKDLTD |
| | | FSLSFKNGDLFVDIYLLSDLVELAKTLISLLINYSNKSQFDSYKNELIDDTYQKAKKYLETFKISFFNSKKEANY |
| | | FYQTRVLSELKGAVALFSKKYYQAKYNIQILKSNEIFPLFVKFSDLLKKEEINDINKLKLIFKKPYRYLIALKKIK |
| | | FKKNQQQSSVLHLDKKNKELVLISSQDEDFLCKLTSSFYQLQFLDRFVYPVKKWLDVDIALSEWSFILEKKYKI |
| | | NWDFNSGKPEFSEIDSRLYLNIPFKIKAINQQKILKPKELFLGIDVGEYGVGYALVNFKDEEIKIIKSGFIGSKNIA |
| | | SIRDKYRLLQDRSKKGVYFSSTNVVQEVRENAIGEIRNQIHDILIKNNADLIYEYNISNFETGSGRITKIYDSIKKS |
| | | DVYAENEADKSVIQHVWGIKKPIASHLSAYGSSYTCSNCGRSIFSFSEDDICSSKVIKRDGNIITIQTPKGEVFAY |
| | | SKDKKFNVGYSFSQEKSKEEMKNLFMKIVKAYARPPLLKSEVFLAQKKLDKEFLEKFKKERGNSAIFVCPFVE |
| | | CQFLADADIQAAFIMALRGYLKKKKGKDINYLEESLNYLQNFKGKINFSNLLH |
| 121 | 2237138 | MFNQKKGYRLHLERIIYSGGEITRSIKYLLASYSDSQKNKELLNNFSQDLYNDDLKIRGCLNLNDLVNNNQIYN |
| | | LADFWIDSLRAGVIWQSSASSLIDLIKRSNHQETIGEKIFNNANERIKRFFNSEKFIKEIILSDPKRIGSKKQAFYN |
| | | SLFDILKDEFKKQEKNEKIILDNQAEQLIKEIIDAFYSNDGTFIMEGEEKQNNFWQEKFNIDKNMIKKEKEDILK |
| | | DVGDITAFIHPSLLSLKSDVSQLIDERKKYFSEKDLEEILGLSDNFNAFSHYFNKFFLLLYQDKQEKIFECYQKIF |
| | | YFSQEDRKRIKGALDFLSEKSKLLGLPKIVNSWSDYRSVFGGKIKSWFSNYLNREDKAKKQEKKIKEGLDKVN |
| | | KFLLNFIQKNQIDPDLQQEIKFYHDKLNQFINSYQNQEFFHQQEIFLLFSDLLAEYKTRLNFFYQKYLSDKEKEE |
| | | KKVDEFPLFKDLFEKYEGPISFYGKTKLEDNKKIIDLTFKTIKVGLNLIRRLLLDLYNSFDFKNSDNNNQERDLR |
| | | RIFEFLLNKIPATKTFREKYLSILKDNFDQQTYKEMTLKPSRYTFVENIYSRENRKLIELPSKNFEESLSKIIKDLT |
| | | DFSLSFKNGDLFVDIYLLSDLVELAKTLISLLINYSNKSQFDSYKNELIDDTYQKAKKYLETFKISFFNSKKEAN |
| | | YFYQTRVLSELKGAVSLFSKKYYQAKYNIQILKSNEIFPLFVKFSDLLKKEEINDNNKLKLIFKKPYRYLIALKK |
| | | IKFKKNQQQSSVLHLDKKNKELVLISSQDEDFLFKLTSSFYQLQFLDRFVYPVKKWLDVDIALSEWSFILEKKY |
| | | KINWDFNSGKPEFSEIDSRLYLNIPFKIKAINQQKILKPKELFLGIDVGEYGVGYALVNFKDEEIKIIKSGFIGSKN |
| | | IASIRDKYRLLQDRSKKGVYFSSTNVVQEVRENAIGEIRNQIHDILIKNNADLIYEYNISNFETGSGRITKIYDSIK |
| | | RSDVYAENEADKSVIQHVWGIKKPIASHLSAYGSSYTCSNCGRSIFSFSENDICSSKVIKRDGNIITIQTPKGEVF |
| | | AYSKDKKFNVGYSFSQEKSKEEMKNLFMKIVKAYARPPLLKSEVFLAQKKLDKEFLEKFKKERGNSAIFVCPF |
| | | VECQFLADADIQAAFIMALRGYLKKKKGKDINYLEESLNYLQNFKGKINFSNLLN |
| 122 | 2237649 | MFNQKKGYRLHLERIIYSGGEITRSIKYLLASHSDSQKNKELLNNFSQDLYNDDLKIRGCLNLNDLVNNNQIYN |
| | | LADFWIDSLRAGVIWQSSASSLIDFIKRLNHQETIGEKIFNNANERIKRFFNSEKFIKEIILSEPKRISSKKQAFYNS |
| | | LFDILKDEFKKQEKNEKIIIDNKAEQLIKEIVDAFYSNDGVFLMEGEEKQNKFWQEKFNIDKNMIKKEKEDILK |
| | | DLGDITAFIHPPLIILKGDVSQLIDERKKYFSEKDLEEILGLSDNFNAFSHYFNKFFLLLCQDKQEKIFECYQKIFS |
| | | FSQEDRKRIKDALDFLSEKSKLLGLPKIVNSWSDYRSVFGGKIKSWFSNYLNREDKAKKQEKKIKEGLEKVNK |
| | | FLLDFIQKNQVDSDLQQEIKFYYDKLNQFINSYQNQEFFHQQELFLLFSDLLAEYREKLNRFYQKYLSDKEKEE |
| | | KKVDEFPLFKDLFEKYEGPISFYGKTKLEDNKKIIDLTFKTIKVGLNLIRRLLLDLYNSSDFKNSDNNNQERDLR |
| | | RIFEFLLNKIPATKTFREKYLSILKDNFDQQTYKEMTLKPSRYTFVENIYSRENRKLIELPSKNFEELLSKIIKDLT |
| | | DFSLSFKNDDLFVDIYLLSDLVELAKTLISLVINYSNKSQFDSYKNELIDNTYQKAEKYLETFKISFFNSKKEAN |
| | | YFYQTRVLSELKGAVALFSKKYYQAKYNIQILKSNEIFPLFVKFSDLLKKEEINDINKLKLIFKKPYRYLIALKKI |
| | | KFKKNQQQSSVIHLDKKNKDLVLISPQDEDFLFKLTSSFYQLQFLDRFVYPVKKWLDVDITLSEWSFILEKKYK |
| | | INWDFNSGKPEFSEIDSRLYLNIPFKIKAINQQKILKPKELFLGIDVGEYGVGYALVNFKDEEIKIIKSGFIRSKNI |
| | | ASIRDKYRLLQDRSKKGVYFSSTNVVQEVRENAIGEIRNQIHDILIKNNADLIYEYNISNFETGSGRITKIYDSIK |
| | | KSDVYAENEADKSVIQHVWGIKKSIASHLSAYGSSYTCSNCGRSIFSFSENDICSSKVIKRDGNIITIQTPKGEVF |
| | | AYSKDKKFNIGYSFSQEKSKEEMKNLFMKIVKAYARPPLLKSEVLLTQKKLDREFLEKFKKERGNSAIFVCPFV |
| | | DCQSLADSDIQAAFIMALRGYLKKKKGKDINYLEESLNYLQNFKGKINFSNLLH |
| 123 | 2237594 | MFNQKKGYRLHLERIIYSGGEITRSIKYLLASYSDSQKNKELLNNFSQDLYNDDLKIRGCLNLNDLVNNNQIYN |
| | | LADFWIDSLRAGVIWQSSASSIIDLIKRLNHQETIGEKIFNNANERIKRFFNSEKFIKEIILSEPKRISSKKQAFYNS |
| | | LFDILKDEFKKQEKNEKIIIDNKAEQLIKEIVDAFYSNDGVFLMEGEEKQNKFWQEKFNIDKNMIKKEKEDILK |
| | | DVGDITAFIHPPLIILKGDVSQLIDERKKYFSEKDLEEILGLSDNFNAFSHYFNKFFLLLYQYKQEKIFECYQKIFS |
| | | FSQEDRKRIKDALDFLSEKSKLLGLPKIVNSWSDYRSVFGGKIKSWFSNYLNREDKAKKQEKKIKEGLEKVNK |
| | | FLLDFIQKNQVDSDLQQEIKFYYDKLNQFINSYQNQEFFHQQELFLLFSDLLAEYREKLNRFYQKYLSDKEKEE |
| | | KKVDEFPLFKDLFEKYEGPISFYGKTKLEDNKKIIDLTFKTIKVGLNLICRLLLDLYNSSDFKNSDNNNQERDLR |
| | | RIFEFLLNKIPATKTFREKYLSILKDNFDQQTYKEMTLKPSRYTFVENIYSRENRKLIELPSKNFEELLSKIIKDLT |
| | | DFSLSFKNDDLFADIYLLSDLVELAKILISLLINYSNKSQFDSYKNELIDDTYQKAKKYLEIFKISFFNSKKEANY |
| | | FYQTRVLSELKGAVALFSKKYYQAKYNIQILKSNEIFPLFVKFSDLLKKEEINDINKLKLIFKKPYRYLIALKKIK |
| | | FKKNQQQSSVIHLDKKNKELVLISPQDEDFLFKLTSSFYQLQFLDRFVYPVKKWLDVDITLSEWSFILEKKYKI |
| | | NWDFNSGKPEFSEIDSRLYINIPFKIKAINQQKILKPKELFLGIDVGEYGVGYALVNFKDEEIKIIKSGFIRSKNIAS |
| | | IRDKYRLLQDRSKKGVYFFSTNVVQEVRENAIGEIRNQIHDILIKNNADLIYEYNISNFETGSGRITKIYDSIKKS |
| | | DVYAENEADKFVIQHVWGIKKSIASHLSAYGSSYTCSNCGRSIFSFYENDICSSKVIKRDGNIITIKTPKGEVFAY |
| | | SKDKKFNIGYSFSQEKSKEEMKNLFMKIVKAYARPPLLKSEVLLTQKKLDREFLEKFKKERGNSAIFVCPFVDC |
| | | QSLADSDIQAAFIMALRGYLKKKKGKDINYLEESLNYLQNFKGKINFSNLLN |
| 124 | 2236768 | AIIAFDHVLKLSQWLSKQIESANSPSSSGSTHTYSPKSLFGLENNFNALSNTLGSVLNSLRGVRSKSAHNTHPEEI |
| | | VKYMIKICRPLEKHKDKLDTIFKTLSQRVKDLEPKFVANWAVYRSFLGGKIQSWFSNFENFFFVDSPDLFTEEL |
| | | GGLKANFSDASNVTVKSSNGSLQTPEPLENQKSERTTAKKDRDKEKLNRSGVVRLCAKKVAVEECMRYLEKI |
| | | GCLLRECPESVKTKIKVDFEGRINQVLVFLENLHNQIKSTCAFLNRGAALIYFGQDLGLSTEMNVSSPFSEAFSA |
| | | YEATIQQIKALTPELNQLTQALDGFFVENFSRTTRRRKSNKKTKQVPKELSGFQTSFEANLKKLKELVQEIADE |
| | | LPKVPYFFGEAASRRHLKAIFAPELAKTLFEGVLTWFEQVNEQLETNSNLELYLQRELSLKEKQRILQIFKSSDD |
| | | FRVFAQKQLKSRFNNQSLANLSLRKAWLQLLHDAKRDPKGQANNNRVYYKSEYSRSHSEVISAPSLNLRQVL |
| | | NFVLFLKSSLLNSFESVPPTFDIPQKAEEGAVLGQVWTRLQKFVVLIRSVLPMFFETSSDSNDSSISSQENNSECL |
| | | SPQMSEFQVPRPLLTLMFKMLKDCLYRVGENLSQKFEAIEEILVQPNLLEDLIGIGCKQLITMNFEASAAVGHG |
| | | GTHAAAPLADEDSLHSLSPFLKDLQTYYFSLIEGLLKVARPRRFLERYVVQFINGGSRESRLAKSKEDEEKYITL |
| | | LPSVPHFFRKASSPKADNFAQELRVTLFDWLFSWLERLYEKSKQSANFEAYFNRGLTPKEKSRLLKILTNSQGF |
| | | VDFAEKVLEASRSPELGFQLEDLTGVWLPCFETVRRLNQHGVSHNLQNKSSNEVDLLLSEIDLKLKDVLAFLIT |
| | | LKQAVFKLLIGREKAVEGQSTSSFDEMALLQKFWLVARSSISTILGISGISSSRGKKRRFARRQSSSAEQSFTSPL |
| | | QTFPVDVFHGILWLLEKSLKEFPSKLKERFKEQDFRSLQNLLAPSFSWVSEDGKLMGDNELDLQSLKEQALQT |
| | | ESTELYLQFLAKYFKVFEARFLVLFEGLLWPAVLSSKGDVVSLIARLKKVDTQGRKNLWDFFVELKKFALGSK |
| | | NLKGFEEHLEPRIRTSRYQLIFLNRFFNRVLRERWPSSKETEWIEPEIVLNEPSLILERWCFVRPKKFEPPEQFEN |
| | | LKKFGDLYLLDLAEQIDNPILFLAQPFTISVSVKDKQV |
| 125 | 2237014 | MFNQKKGYRLHLERIIYSGGEITRSIKYLLASYSDSQKNKELLNNFSQDLYNDDLKIRGCLNLNDLVNNNQIYN |
| | | LADFWIDSLRAGVIWQSSASSLIDLIKRLNHQETIGEKIFNNANERIKRFFNSEKFIKEIILSEPKRISSKKQAFYNS |
| | | LFDILKDEFKKQEKNEKIIIDNKAEQLIKEIVDAFYSNDGVFLMEGEEKQNNFWQEKFNIDKNMIKKEKEDILK |
| | | DVGDITAFIHPPLIILKGDFSQLIDERKKYFSEKDLEEILGLSDNFNAFSHYFNKFFLLLYQDKQEKIFECYQKIFS |
| | | FSQEDRKRIKDALDFLSEKSKLLGLPKIVNSWSDYRSVFGGKIKSWFSNYLNREDKAKKQEKKIKEGLEKVNK |
| | | FLLDFIQKNQVDSDLQQEIKFYYDKLNQFINSYQNQEFFHQQELFLLFSDLLAEYREKLNRFYQKYLSDKEKEE |
| | | KKVDEFPLFKDLFEKYEGPISFYGKTKLEDNKKIIDLTFKTIKVGLNLIRRLLLDLYNSSDFKNSDNNNQERDLR |
| | | RIFEFLLNKIPATKTFREKYLSILKDNFDQQTYKEMTLKPSRYTFVENIYSRENRKLIELPSKNFEELLSKIIKDLT |
| | | DFSLSFKNDDLFVDIYLLSDLVELAKTLISLVINYSNKSQFDSYKNELIDDTYQKAKKYLETFKISFFNSKKEAN |
| | | YFYQTRVLSELKGAVALFSKKYYQAKYNIQILKSNEIFPLFVKFSDLLKKEEINDINKLKLIFKKPYRYLIALKKI |
| | | KFKKKQQQSSVIHLDKKNKDLVLISPQDEDFLFKLTSSFYQLQFLDRFVYPVKKWLDVDITLSEWSFILEKKYK |
| | | INWDFNNGKPEFSEIDSRLYLNIPFKIKAINQQKILKPKELFLGIDVGEYGVGYALVNFKDEEIKIIKSGFIRSKNI |
| | | ASIRDKYRLLQDRSKKGVYFSSTNVVQEVRENAIGEIRNQIHDILIKNNADLIYEYNISNFETGSGRITKIYDSIK |
| | | KSDVYAENEADKSVIQHVWGIKKSIASHLSAYGSSYTCSNCGRSIFSFSENDICSSKVIKKDGNIITIQTPKGEVF |
| | | AYSKDKKFNIGYSFSQEKNKEEMKNLFMKIVKAYARPPLLKSEVLLTQKKLDREFLEKFKKERGNSAIFVCPF |
| | | VDCQSLADSDIQAAFIMALRGYLKKKKGKDINYLEESLNYLQNFKGKINFSNLLH |
| 126 | 2237881 | MPPIQPAQKKHPKAVKYLGQTGYTLHASRFSYTGMGVMRSIKFPLYHSERKVYDTFCQQIISDYKLVQGATNL |
| | | ESFLVDEQSYSLIDFWVDSLRAGVVFQPTEAQMSAFLRQHYQMTDVLEIAFSNMDRELRDKLDYLAFRQKIM |
| | | LSTGLRPTSKFPGFLKCFKPDLQKDEAVIQLANQIFNHLVHDGVALTQSEQKEYWLSNWKLNLPEKSSTGESS |
| | | TFFILPELQLAQDMTVGQCLDQRRQLFPDQMLTILGFENTFSAFSNYYGKVITELRSGDVENIEKTIKSMSSSW |
| | | QGNEVELKKRLEFLSARAQLLPVSYLAKYPDYRTSFGGKLQGWYSNYEAQIKRIGEQIGQFGEAIQKAIDDVR |
| | | VVGVHENDVGFQESHEQRKSVLLTNLDQLKQLQLQLADADNRDFVTLELFRDLLSYVRTDLNWIYQTYLQT |
| | | DKEKKEEKMVESRYRGLFVKLKLVPEFFGDSRKKQFAKYRDQVVPILRDGQAIVDKLKTELLTSPDYSFVDQ |
| | | QFFVKQLQKLLDKLQTKSWNTKKYQQMVEEVVKKYNNGLMPGRQQAFFLSPYARNKHCQVIQIGVSLDWG |
| | | SELSDLIKQLNHSFSLDQNPTELQDWLETQKWLIGWLLRANPNLEIDSAEWPTQVFTKARTFQQQFGTKLSGG |
| | | GRNYFVNSLIFSEMRGAASSFSRREIAMRYVVQPIASLNKYPLVLKTKQENPDLKDKHGLVNQPHRWYLASQ |
| | | KPISKKESTFESEVGLVTEDNDGKRFGLLRSAYFDPGTLLEIRTSKYQIHFLDRLLYAGRKWCDLDVTASDHSL |
| | | IVEDTYRVNWNLQTGQPGVQKIDENRRLYVSVPVNIKPVLDNDEKKVKKQAIKDGRQRYLGIDVGEYGLAW |
| | | VVLDFASSLNPRRIAQGFIFNQSLRKIKDKAEEIKDTQVTGTFTVPSTKLARVRENAITSLRNQVHDLVIKYNAK |
| | | PIYEHSISNFETGSNKTTKIYRSVKVSDVVAENPADDAVKSHVWGSKYITVGNHISSYATSYTCSQCHQSIYDQ |
| | | GLESQSEVKRHVVKEIYGRVIIVQLANNRAVYGYVPPKRDEVCFPYHTGSQLSDKEVYTLVKNFARPPLDKSE |
| | | AVSFVKNKKPNQLPEKELSLLQESRGNSSIFICPFVNCLHVADADMQAAQNIALRGYLKDQEPESIRKKREKNR |
| | | EAFDYMGAVNMYFKTVKLESMNLEIRT |
| 127 | 2236658 | MAKRVSLSGYRLHVKRLHFASEAPTVRTFKFPLVRKCGKKTDFTDVPQELIEAIKHDDLYIRGPSNLVDYLRF |
| | | KDRKIYTLIDFWIDSLRAGVIFWQDARRLRTDITRIYSVSFLDEYLKEKSPKLFKYFKPADFLNKVVIKRIDLRK |
| | | QGKKEKLKKLLISLIRDEYKNNNRSEGELGGEDIVVDLEAKNLVDDLTSNLLKNGQFLPEKDRVSYWLTKWN |
| | | IYVQELKEITEYDFTALIFPEISLDLRVEVNEENVLEILNQILIQRLIWIAKTPGTVKEDTRSLLLKYSDELRKAVA |
| | | HPNLLSEILNEAWEDIYKEVYKEEARNSQRTKNDKKDELEEKVIKKTNSEFKKKIVDPVANALKELLGLSNKA |
| | | SALSNYFNEFLEYLKKSNTELIINYYASLGLPKKKLVEIREKLFWLSEKAKQFHKSAGKPKLSKLLGKGINNWH |
| | | DLRTVFGGRLESWLTNHIERLAKQYRQIVILKKSLPKIIEFTNKYPDDYRDINPDEINNRKQEIQGLIEEIKAKVG |
| | | NPSVVQDNLEDYEVLEHLVKALRQRVNEYFQLYFRDDLERRNTKLSQVKELKDFWSCRVYSPRSFYGYAQR |
| | | KRLRKIIETTIPTIKTGISLTRYFLKTLKELPDYTKATQEKDKRKVREDAGIRRLLEILRSKYKDKKLNSDKFKDF |
| | | YRELLLRYATGKDLASNSKTFYRSEYSTSTQQEIELTVSDYAREVEKIVTEITEFVLSVTDKELLNDAFLLLDWI |
| | | ETARLVVARFFRWMDQEKEWDVTKFRLSTFPAANKFIGRLKNKQVKTPMFRFIIMSFVFSEFRGAATVFSKKY |
| | | LISKWNVQVVGSDKKFKLALAPKNQDVDIERMYQLIVRRRPEVLKNIRWQVIIYTKQGNSQQLITLKNKSGSF |
| | | SLSKEKINDTNLLQICTSKYHLQFLERLIYQPQNWQDIEIKISEPSLIVEEVDEILGWDLNSGRPELKKLEKNRPIL |
| | | YYALPMQFAVKRAENTITQLQKIKQAKPEDVYHLGADIGEYGIAWAVIKIENGQVVIRDYGFEYDKQIRKIQD |
| | | KFNEVQQKARQGVFLEASTALAELRENAIGYLRNKLHNKLIDYGAQIIYEANISGFEVGVGRVTKIYDSVKRA |
| | | DVGNHPDVSTKIYKSLIKHIWGLEAPRAKGDRMRDKSPGWQVGAAGTSYTCICCGQSIYQLGNNKKNKLKED |
| | | KDKKDK |
| 128 | 2237521 | MPRSPKRKPAYNGRTGYALHTARLSYVGKEGTRSVKIPLDGHQETVDTFWDHVKGDYIGQVGEMNLADWID |
| | | GERSGTHGYSLADFWLDTLRVGGIFSHKEHDMRAIIAHMNGDTSFEKRFVDAVTERHPTLASLADTRALYNFF |
| | | ISGSRNGNKTPDQIFQKVRNSVFPAYRNIEHTEVDAHTRRLVVASSIEGKTGDGDSGKIIYAKELVGFDPTPDIP |
| | | YANTFFIHPGIPVRFGASAKELLFSYRTFVTQIHPELYGFDGENENDTLKKAFGLENNQGAFSQYFNAILADLRT |
| | | GGSTLEHAIKAVSPYWRDHDEDLHVRIAYLAEKAHMLPEPYLAKGWHEYRSTFGGKIESWVSNVLRQEQEIR |
| | | GQLFGEMKKSKKRNDKKEAFVPGHKQVLESALADAILSDAVKKNINDCALLVSRMETSGVTDADLLLYRAIL |
| | | GDVRIGMNTDYQMAYPVLDGKYEKERREEEKTKRAYRKYAPLYKDIKLIPNFLGDSKRIIYKKFVRSAELLHT |
| | | GVAAMAYAEDVLATAPFSPRFVSDTDADAFLCKLYDVLRRKYGTLNATRFRGVIEGIFRNLKVSIDGESENSA |
| | | SALLPDNAERNGITYVCYMSPYARPNPRLKYADIAITTEEIPKVLDTLLVALKPYWNDVMQSGDMGELADAV |
| | | EMEKIRAGIIVALWYKNTLSTQDILAKLPEELFESAHAFARMKGNPETVSGDDMGRLLQTGILSEMKGAIAKM |
| | | SRMEYVERYTNQRMNSEEEMPLCKTDTGWHIASEKTNKKELAGLVPVSAKKGKVFSGEGDYVSAFARPDTL |
| | | FRLNTSAYQLQFLDKTLPHGNRATSWWNAHGVDFSLASYSFIAERPVYIHWNVAGGTFSLERSENKRLFVSIP |
| | | FSLSPEKKPADVLEHRTRYMGIDVGEYGLAWTIIETDREQKKISRILKQGFIYEPLTHAIRTYVGSLKQQQVKGT |
| | | FGMPNTKLERLRENAVGSLRNKVHHIAMRYDAIPVYEFEISNFETGSKRTTVIYNSVKRADVGRGGSEADKAD |
| | | ANLVWGKNGKNFGKQIGAYATSYICSRCGYSPYEAFQTSGVRETLNDEKKRSRPSLRDFLTTHPAFATHEAFV |
| | | SRGDKESAWKERRGNSGIYVCQRCGHITDADVQASYWIAAKSFANNLRGKKEQTDADSEKIPPVSFKELLLV |
| | | HGIDDDHVADNKHITRLNTRL |
| 129 | 2236756 | MFNQKKGYRLHLERIIYSGGEITRSIKYLLAGYSDSQEHKELLNNFSQDLYNDDLKIRGRLNLNDLVNNNQIYN |
| | | LADFWIDSLRAGVIWQSSASSLIDLIKRLNHQETIGEKIFNNANERIKRFFNSEKFIKEIILSDPKRIGSKKQAFYN |
| | | SLFDILKDEFKKQEKNEKIILDNQAEQLIKEIIDAFYSNDGTFIMEGEKKQNKFWQEKFNIDKNMIKKEKEDILK |
| | | DVGDITAFIHPSLVFLKGDISQLIDKRKKYFSEKDLEEILGLSDNFNAFSHYFNKFFLLLYQDKQEKIFQCYQKIF |
| | | SFSQEDRKRIKDALDFLSEKSKLLGLPKIVNSWSDYRSVFGGKIKSWFSNYLNREDKAKKQEKKIKEGLEKVN |
| | | KFLLDFIQKNQVDPDLQQEIKFYYDKLNQFINSYQNQEFFHQQEIFLLFSDLLAEYKEKLNRFYQKYLSDKEKE |
| | | EKKVDEFPLFKDLFEKYEGPISFYGKTKLEDNKKIIDLTFKTIKVGLNLIRRLLLDLYNSFDFKNSDNNNQERDL |
| | | RRIFEFLLTKNPSVKIFREKYLSILKDNFDQKTYKDIILKPSQYTFEKNIYSRENKKLIELPSKNFEELLSKIIKDLT |
| | | DFSLSFKNDDLFPDVYLLSDLVELAKTLISLLINYSNKSQFDSYKNELIDDTYQKAKKYLETFKISFFNSKKEVN |
| | | YFYQTRIFSELKGAVALFSKKYYQAKYNIQILKSNEIFPLFVKFSDLLKKEEINDNNKLKLIFKKPYRYLIALKKI |
| | | KFKKNQQQSSVLHLDKKNKELVLISSQDEDFLCKLTSSFYQLQFLDRFVYPVKKWLDVDIALSEWSFILEKKY |
| | | KINWDFNSGKPEFSEINSRLYLNIPFKIKAINQQKILKPKELFLGIDVGEYGVGYALVNFKDEEIKIIKSGFIRSKN |
| | | IASIRDKYRLLQDRSKKGVYFSSTNVVQEVRENAIGEIRNQIHDILIKNNADLIYEYNISNFETGSGRITKIYDSIK |
| | | KSDVYAENEADKSVIQHVWGIKKPIASHLSAYGSSYTCSNCGRSIFSFSEDDICSSKVIKRDGNIITIQTPKGEVF |
| | | AYSKDKKFNVDYSFSQEKSKEEMKNLFMKIVKAYARPPLLKSEVFLAQKKLDKEFLEKFKKERGNSAIFVCPF |
| | | VDCQSLADADIQAAFIMALRGYLKKKKGKDINYLEESLNYLQNFKGKINFSNLLH |
| 130 | 2237128 | MLPFGAGALIIILNMQKGYNLHKKRIDLLKGEILRTIKYPLKPLNENSEKNKKQFELQIINDDLKIRGDLNLNDY |
| | | LNYTFKNNHIYNLFDFWIDSLKCGVIWQPSFNNLKKLIEKIYLNKTISEEIFDNSIPEIKKFFDKNKFIENFILKTGI |
| | | RENRSKKSFEKDLIKSLNDEFNKEEINKEAQDLIDKIINTFFNENKLKKLTIEEQNNLWLNKFNIDKSKLKTKKL |
| | | KYNYTFFIIPELINFEENNLENLLNIRCEFIKNKNLNIKILQEILGLENKFNSFSNYFNKIIKNLQNDKYNLIYEDFI |
| | | SFYKFTNEEKEKILQSLKYLSEKSKQLGSPQILNLSWADYRSILGGKIQSWFTNYLQRKEKLEEQLNRLKENIN |
| | | KTKEYLLKNFQKNTDLKEIIYFLDKLLELSEVSKINNLFFDIHSTCKLKINNFFQKNIKQNEDDTIESNENLKGIF |
| | | EKIYKPLNFYGQSRKENNIKIINSLQTVELGIIYTKKILNFLKNDLDFNKEENFEEIYRKNLNFLWNKLKENSLNS |
| | | KNFKSKYKEIIIKNLNSYSNIKFEKIIKRPSYYVFYKSIYSKGTNLQIEFNKEINFKNIFIENINELYNFLINLEDKEII |
| | | IDKNLLLDWVELSKNISSYFIKFTNIEKINLKEIFKDKDLNFFSNIAKYFETFGNVVEKQGLSYFIQSIFFSEFKGA |
| | | ATLYTKNKYLAKYNIQIIDSNKKFPLYYYSNNLKNHNKLLNPKDLIKEKHNYFVSLKKIKSKNKDLEFLLLNK |
| | | DNFEKIYNSFEDNKNDLLKLESSFYQIQFLEKFIYKPKNWKNSKIKLSEWIFILEKEYEIKWDLENKIPILNEIQNS |
| | | KKNKLYIAIPFKISSDQKEYEEIKKSKENYILGVDVGEYGIGWVLINYKNKELKIINKGFFVSKNIAKIKDEFSKI |
| | | QKKSQKGIFNESSNLISEIRKNAIGELRNKIHSVFIFYHSKNICYEYSINAFETGSGKTTTIYKSIKKSDTLSGIDAD |
| | | KKEQKHIWGKELNKIEIGKNLSSYASSYICSKCGKSLYQLKETDLENNEIKIIEREGNILKIKTPYGNVYAYSSK |
| | | KEHQVDFIFNKTKSSFKELQKIIKDFARPPLNNNSEVIRKFTNLKENEIDILHQKRGSSCVFVCPFCLHVSDADIQ |
| | | AALMMSIRGVINNETKNKGEKIDYFQKNLEFLNELKEKPVIDL |
| 131 | 2236760 | MANRQISPVDSTNNFIFTLYVSFDTVLYARILAFLTLSCLLVGFLEYIYPLGVIYLIMPTNQQYDGLTGYQLHTE |
| | | RLRHTGKSGIRTFKLPLKTNDQELFGNFFENIKHDHEGQIGATNITTWTEEQKSSRQIYSLLDFWLDSVRAGVV |
| | | FASSSAELNELLSVCGDEDAVDEQVKTAMRAINPTFFKHLKFNDFKEEIALKEGMRSSSTKNSVTRRLYKCLD |
| | | CTEEDAPEEIQQAVADLIKTFFTTDGKIISRNDQDDYWQVHFGLDKSLYKLDSDIKLTFSFLPDIPFTPEADAHK |
| | | CLDKYRCWIDENAEKFQLESKEDKTNFLQIHWGIEGNYNAFSNYFNQIIDLLREEEGRQKLLDALLNTSDLWE |
| | | GAETELQKRFDFLVDKAKQLPKAKCVDSWSDYRTTFGGRLSSWLSNTLNQEEFIKDTVSDQKEELNQKEELK |
| | | QIIKNSEINLYKKAFSAADDNRAVELSHEAVIAQKTLEKLQKSSSFEPQLLGVYRDNLGRLRSLLNKAHQLLPD |
| | | EIENKSAHEVYSALHERIRLMPKFIGGAKAARFEKYVKSLQILKQGASFLENFEEKVKEAMKSSVSVEVEEISE |
| | | GYFLRQLNTLKRFYDNANDSRFKGKLSSLFDKDLGVNIEAVSNRETFYISPYSKHDNRAVISVEVANYQQLLK |
| | | SWVNELKPYWGNIIATENWGEIIDAMQLERIRIGWIYKLYPKLTFAISDDLDELFAKAATYRDLHGHEVEGSLA |
| | | ANFLQRIILSEIEGRVKEASREKFTVRYVIQPVGSEKTTKYPLFVTSNKDKLSATERKQWYIRDINSSFSRSETDK |
| | | KGWILKNGKDFSNGFQEKYISDNTDLFKIISSKYQLQFLDKALPNGAKKFGKDGDVNLKLSEHSFIAEETLQAS |
| | | WDFSTDTVSLKSVDKDLFVAIPVNIEPESQRSSREERTRYLGIDVGEYGLAYTLIETIPKVRVIKSGFLYEKTLRN |
| | | IRKQVTNIKNSQRLGTFGVPSTKLARLRTQAITKLRNKVHDLVVRYDAIPVYEWQISNFETKSGRVTKIYNSVK |
| | | RADTNSGANENADGDEAALVWGKRPYKIGQEVSARATSNMCSNCFKSIYQFTEQDFTEESRSGEYVEFKIAK |
| | | NTVKGFVPNDEKITSKQKMINAVHDYARPSVDILVKRNDVGVDKEILVNFLEHRGASALYECPFCSHLSDADV |
| | | QASLFIGLRAICRDQDDETTKVKISDLMDFAQKQNIDPISADFIQ |
| 132 | 2236848 | MGNVQSTRAEQNATAPSRQPKIKHPPSEKGYTLHDRRIAAAGGDKAIRSIKYEVVFADQDRQRVIEAIRADHL |
| | | AINGPVNFRDFVRHEIEGTPRKVSFVEAWFAMLRAGAVFQPATGALTNLAARVRGGSPLKKFRDTFRPLLIAE |
| | | YAELIDWAELFDFVSKGTRDTTNGQMGGKILRCFSADAKESTDPATVAIIKRAMDSFERVYPLIRVFKGDGEA |
| | | KKRHELRQKIAIFLKEPPPAEDGRGAASVAIQDSPTVLLARGPGVPAAASRMITATAIEKTLSEIEGWASALSAG |
| | | DVEEEAANALSLSTLLGLGGAGFSSLANFGNDFLPGLADQTKLPNLAATQAALLGQDAAGLAQRLAAVQASL |
| | | GDVDWNMQLANGPGDYRTDIGGKLQSWLSNRDRCRKELATALDELLVELQDKAAVLLEHYSDATKVGQEV |
| | | ARWAARTTVVKAAVTGLTTRIPLAKGAIEQNQENAQELLDLLADDIAALNQELNEWFQVVVESRRRQGPPAG |
| | | RRSRSPGGRRGEASFSVEWFESRPADELDFRKIFKQLSKRLQRIPLFLGLQQQRTYVKAYWAPRLVRVLHNFL |
| | | LADLKALKEGATPSALSAPEKQRVLAWWRKVRDQCIGAEQARFARMVGAAAPLHPFQKVPDDAVIYKNPRS |
| | | RARVRVLAESDHPALGSHDIIDWLLENLSLGLPALTQHGWDLYLALVELAKSFIPILLERSSAIDRRIDAARLAV |
| | | DVEGRWWGPAPFEKLKVALALRGGETGALSTQDYQYLVNLLCTELRGLVGILSRKEITQNYTLQSINGSQSDL |
| | | LVAAAQAVDVAPDKARFMLHLPRLLRGAAAEPTPPLLRWIKGNESTVIVPTAPRGVVGEIRSSKYQVQFLRW |
| | | ALAKNLEKRWGKKRCQLEVSGTSLVLEERVPLKLDWSTERLTPLHDQVTSRIFSVVPFSFIPPTSQEGLLPISSN |
| | | RFIGVDVGEFALCYSGIEVAGDKGSADVKVLGSGVIFDPQLRKLSKRVRELREAQRGGNFSYRSTRVARLRES |
| | | LISSLRSRLHAVLLRYGATLVFEFDISAFEVGGNRIAKVYDSVKRTDTIPDNDAEKAERKHCWGRTNFRSGAE |
| | | VGARGTSQTCTKCGRWAKAHVKEHLSGPAIKASALVREDSNVYAAKVGAASLRLFLKQPPGPEALLDEGALT |
| | | TAIHDYMRPPLAQASAQIGDPVAAAAIRGNSSFFRCPFADCNHVADADLQAAFNIAMKGACAQLWKHKGDD |
| | | WLLQFAAEGRPAVGIDSKDYAWPKGS |
| 133 | 2237517 | MQKARNGSNGWKTSSRHARTGYRLQLARLQRTGKEGVRSFKFPLMPYGGTGSELSAFAKSVEADYRNSVGL |
| | | LNLSDWYENEKSGGGHYSLVDFWLDSLRMGVIFAHKKVEMKNTLLLLNGDTSLRKRVLDAVWRRDARLYG |
| | | IADKENLYQFFISPSRPGKKTKEVFAKKLNGDLFPSVPESDVAHASEYVSRFSDWVFSGTKQAQVTALLGFDP |
| | | KANHSYPYMFFFSPHLSRQEEASPKQLLATYHSYLRDDRSPYREHDQEEQIKRVLGLYNNHGALCNYFNKAL |
| | | KKLQSKDSGAIVQAVRAASPYWSEHERELCTRVEYLARAAQKLPKATLDERGWHQYCSSFGGKLESWVSNTI |
| | | RQERLLRAELFSGEAAGEMSTEQDAQRFKKKKRKEKEEKARSSHEAALSAALADPILKEETRELVRQCQSALT |
| | | QLRTEISDDLLAAYRLLLGELRTALMQDFQLATFDDSEPGEEIKKLQAPYKPLFARVKLIPNFMGDTKRSVYEK |
| | | FIRAPRQIKEGIRCVTACADTLKQSACDPRISDAELVGHARKVYETLRRKYPALNATRYKHIVESLFAARRGVL |
| | | EDGTPFALSRLLASADDRRNRLDANRFVLWKSTFNRSQAARVTLDISADAAPAILKQLAVALAPRWDDIVVT |
| | | NDFGELLDACEMEKIRLGILVTLHHKTAVSIDIDALSPDLFGSAHAYFSFLGKPASLAGETLGRFLQAFILSELR |
| | | GALSKMSRTEHVERYVVQTLRSEEHFPLCLREHAGKRQWCIAAERPAVLRDRHTQCPEGKVAVQLKNGKDF |
| | | GNKNEFAAACADPTSLLAIRTSVYHLQFLDKALGGSSSWWSLRNFNLKMGEPSFIVERTVRTTWDIVKEQVSF |
| | | ERDEPRVYASIPFTVQGATHTAPLDERPRYMGIDVGEYGLAWAIVERGSGAKVGKIVASGFLFEPLTHEVRNF |
| | | VQTLKAKQSLGTFGVPTTKLERLRENAVTSLRNQVHDIALRYRATPVYEEEISNFESGGNRVKVIYNSVKRAD |
| | | VERSYDDDAGTMEADLVWGKKAKQFGKQVRAHATSYICTKCGRSPYEEQDKAQKEKMRGDSRPSVKDFKN |
| | | LESKQFTDWQQHPLLRQNSEGVSPWEERRGNSALYVCQYCNHVSDADRQAAYWIALKRILRDKDKRDKDK |
| | | KADADKEATNEKKFDLKKDVAALAAAHRKSGNPPVELKL |
| 134 | 2236651 | MIISKKSKKHLGQTGYTLHSHRLSHTGRSAMRSIKFPLFHTDGFASISGTGHKLITSIKSDSELVQGIGSIDSYIAD |
| | | TKAYSLINFWEDSLRAGVVFQPTEATLQDFLGKFSGSQDENQLIFKNFRYDFKYALDFEQFKKKILLSVGLRST |
| | | SKKSSLINCFKPEHRKDHRATQWVDTIFNDLFKDGKSMSGDAQIIYWREKWGLEKKNASSSSIKLTFTILPIFAF |
| | | SATKTLEECLSERRKLLSKESSYDINSILGFANTFSAFSNYYGEVVSAFRTDNFEGIYNALCDQPLGWLKKQKD |
| | | LRERLNYLSLQAKKLKEPRLATSYAAYRTSFGGKIQSWFSNYDSQLEKIYKQLHDHAQDLAQAQKDLSQIEIE |
| | | ENEAIEQRYYFLHDSLAQLTLLQKKLTGQKSMNITLIDTYNDLLSLVRGDLNWFYQHYLQTEVEIKQEKNIEN |
| | | RYKYLFSKLRIVPDFFGHSLAEKSNKYICKTIPQIEGGLLMISVLKQKLLDTSNFSFADEIFIRQQLNTLLRKVTT |
| | | KSINTKKYIVIFEKALEVFSGRKSFNNDEVFYISQYSRQKNLKIIQLSASISKDSLLHLISILDNPFLLNQDNSAEL |
| | | VDYLETQKIITSWMCRGYTEAEIDISGLDFENFKKAKTYLVQFGSKIPKNRLSYFLSTTIFAELRGSASQFSRKYI |
| | | LSRAVVQPMETLRNYKLLLQTAMDSSCSRLDIVQNPHRWYISTDKQLNTGDAVSKNFIMSGENSGKKFSTLKE |
| | | GNYNPESLFAIQSSKYQIQFLDRLIYQSRKWHDVDLLASDFSFILEQTCEIQWSLTLKRPTLKIKNELTKLYASIP |
| | | FTISPIVDKKELDEKKSFLKSRKKYLGLDVGEYGLAWVIMDFTQSKTPVRIKSGFIFDGALRKIKDRANEIKDLQ |
| | | MKGTFSVPYTKLARVREQAITSIRNKVHDLVLKHGAKPIYEFSISNFETGSNKTTKIYRSVKTSDVSTSNDTDA |
| | | DRAVASHVWGKNAVKIGNHISAYATSYTCSECWQTLYELDLSGKKEKTLEKVRQVEKNIYCATVNDKQVYA |
| | | YLPGISSKNLEKILSEELLKGIRSFARPPVEINEALSFAIKSSKKSISKELLSHFKSKRGNSALFICPFCLHISDADIQ |
| | | AAQNIAIRGYLKDVQLLSKPTEKTNDFNYISEVKKFSQNSKLPSVDLEDFEYS |
| 135 | 2237293 | MDKKSSNSTGYRLHKDRILFSGGEIMRTIKYPLVVEKNNLNSEEIVEKIRQAIINDDRVIRSDINLNDYIEYTKK |
| | | GNRLYTLIDFWQDCLRAGVIWQPSTSFLLYLINKLYSKPKAIELIENAKPDISRFFDVDKFSKCFILPGEIREGKIL |
| | | KTFKRELIEALKGEFKKGKKEKIKDEDDYLEKFVEKDARKLIREIADCFFSNDILVTHDLKEGKKEYQDRLWE |
| | | EKFGIKKGKLLENFKLPDHLRNFKNISFFIIPELSDKSKNFDELIELRRKWLLERKICVREDGDYLENEKKLDEEL |
| | | RNLVGLSDNCNPLSNFLGTVFCELLVPNNLNEDNALEKFYDVFTIVEPKIAELNIKDQIMGSLEFLRLRAKQLG |
| | | SPNLVNFSKSQNLKANESIKLDGWSLYRQNFGSKMQSWFTSYIERNKLLEDSLKNFKEKIKKAQNFIKNLKNIS |
| | | EEPQQEEEAQQEKEEIVELFEKIFSSLEKVNRENFEVFDSLLSSLRKRLNFFYQQYLYNEAKEGDDVKKHKILG |
| | | PIFKNIEKPIAFYGETQRKKNEKIVKDTIPILEEGTVFLTTLISNLLDSFSPKQVFPDVRKKDETEEIIYRKELQFFW |
| | | NKLKDLAVNSKEFEKEYQDIIESAVDESELSKLKELFVNKKKNGSKYNKYTFYKSKYTKGSIEEIKLKGSKEEY |
| | | LLRFEKLIKSLTNFLTQFNRNKLLQDKDLLLDWVELAKNIVSVLIRFSTNTEFSLNEIKAQSQFKKAKNYLELFK |
| | | LKKAKKKEFGFIIQSFILSEIKGAATLYSKRKYIASYSVQIVGSNNKFKLFYQPLDSSINISGGPKDFVTKKHKYL |
| | | IVFQDLKNVKNKDATENRINLLRLNKERKIPLVAYKDDLVSKSLLLSSSPYQLQFLDKYLYRPRGWENIDIKLN |
| | | EWSFVVEEAYDIEWDLNSKTPKLIPSPKSNRNKLYLAIPFTLKGNVKEPPLDKIVLKSETKKDHSRDKNRLNYP |
| | | ILGVDVGEYGVAWCLTKFDYNQDFSLRDIDIQGKGFIEDRNIGKIKDYFAEIQQKSRKGAYDEDDTTIAKVREN |
| | | AIGKLRNAIHSILTGSLEGASPVYEDAISNFETGSGKTIKIYNSVKRADTEFKSEADKAEHSLVWGKKDRNQET |
| | | KYIGRNVSAYASSYTCVNCLHTLFKVKKEDLSNIKILEKDGRIVTMSSPYGPDKKVRGYLSEKEKYEIGYQFKE |
| | | SEEDLKAFRKIVRDFARPPVNKNSEVLEKYAKEILAGNKIEEFRKKRGNSAIFVCPFCQFKADADIQAAFMMA |
| | | MRGYLRFSGIVPSKENSKNNPQESEDKSLKNSKKQSETGDTFLTKTAEYLQQLRFEIKEKIKEAVKVDF |
| 136 | 2237906 | MSDQGSAQYDGLTGYRLQTKRLGYTGKSGIRTFKLPLKTDDRELFDTFFADIRHDHERQLGATNITSWLDSKR |
| | | NDRDCYNLLDFWLDSLRAGVVFAPKASALNQLLAQLSGSSVDERVKESMRAANSAFLDALDWNTFREEIAL |
| | | KGAKRSTATPDSFRRDLIGCLQESEEKTPDTVIADVEAIVAQFFTDSGDTIEGKKQKQYWQEKFGLDKSLYDL |
| | | GGDTALTFYPVADIEFTPEDEAPDILQKYRQWVSNNADTFSLESPEAKQEFLRRRLGNLQESGNYNAFSNYFN |
| | | EIINTLQDEGGLDKLTQDLLAVSNMWSDQKEELRNRLRFLQERARRLGQPKCTKSWADYRSVLGGRLASWLS |
| | | NTFRQEQFINETIKKQKAELADLQNECANGVFVDIFSADDDDRDDGLENYLNTARDLLDDLQGQYGFKPEVL |
| | | KVYRDTLGRLRSLLNRASQISKQKDFTADSYPGLYERVRLIPRFVGAAKRERFQKYRRSLITVKKGIDYLQSVS |
| | | VAIDSAEADIDQGYFLRQLNALKRFYTQANSERFRHIVADLFTAVGVDADALGADDTFYISPYSNSTRGRNLIE |
| | | INDLPDTPTATLQAWSDKTAPYWEDILQTQNWGELMDAARLDRIRWGWITKILGDKLQVSLIDNLDTDFPQT |
| | | KVLRNLYGETVSGQTAGDFLQRVILSEMGGRIKEASREQFTVRYVVQPMKTNEQYPLWVTADDDGLTAQSR |
| | | DQWYVRYFQQDFSGDESDQSGWTIGAKGKKPQEGFEQAFLPDDADVFAINTSKYQLQFLDNVLPGGAKRFR |
| | | DDGGFSISFSEHSFIAEETMQADWDFTADRVELESVDKDIYTAVPVQIDPKESASARDQRSRYIGIDVGEYGLA |
| | | YTLLETEPELKVVKTGFIHEPTLRNIRTQMTNIKESQRKGTFGIPSTKLARIRSQAVTKLRSRIHDLVVRYDAKP |
| | | VYEWQIQHFETGSGRVTKIYNSVKRADVSVTVQDDADSAEAELVWGSNTWRVGMDINAYATSNMCSSCYRS |
| | | IYAYESPSFSLIDTDEDYAQYEIETDVSAWAHLPSPDPETADSDEIIRGIKEFSRPGIDVVANRSMNSCDFDHKQ |
| | | VQKLVGKRGNSCLFTCPFCNHVADADVQASLFIALRGLLRDKKSDEESVDVEELLASAQQEGLQVAGDIGS |
| 137 | 2237205 | MNKKSSNSTGYRLHKDRILFSGGEIMRTIKYPLVVEKNNLNSEEIVEKIRQAIINDDRVIRSDINLNDYIEYTKK |
| | | GNRLYTLIDFWQDCLRAGVIWQPSTSFLLYLINKLYSKPKAIELIENAKPDISRFFDVDKFSKCFILPGEIREGKIL |
| | | KTFKRELIEALKGEFKKGKKEKIKDEDDYLEKFVEKDARKLIREIADCFFSNDILVTHDLKEGKKEYQDRLWE |
| | | EKFGIKKGKLLENFKLPDHLRNFKNISFFIIPELSDKSKNFDELIELRRKWLLERKICVREDGDYLENEKKLDEEL |
| | | RNLVGLSDNCNPLSNFLGTVFCELLVPNNLNEDNALEKFYDVFTIVEPKIAELNIKDQIMGSLEFLRLRAKQLG |
| | | SPNLVNFSKSQNLKANESIKLDGWSLYRQNFGSKMQSWFTSYIERNKLLEDSLKNFKEKIKKAQNFIKNLKNIS |
| | | EEPQQEEEAQQEKEEIVELFEKIFSSLEKVNRENFEVFDSLLSSLRKRLNFFYQQYLYNEAKEGDDVKKHKILG |
| | | PIFKNIEKPIAFYGETQRKKNEKIVKDTIPILEEGTVFLTTLISNLLDSFSPKQVFPDVRKKDETEEIIYRKELQFFW |
| | | NKLKDLAVNSKEFEKEYQDIIESAVDESELSKLKELFVNKKKNGSKYNKYTFYKSKYTKGSIEEIKLKGSKEEY |
| | | LLRFEKLIKSLTNFLTQFNRNKLLQDKDLLLDWVELAKNIVSVLIRFSINTEFSLNEIKAQSQFKKAKNYLELFK |
| | | LKKAKKKEFGFIIQSFILSEIKGAATLYSKRKYIASYSVQIVGSNNKFKLFYQPLDSSINISGGPKDFVTKKHKYL |
| | | IVFQDLKNVKNKDATENRINLLRLNKERKIPLVAYKDDLVSKSLLLSSSPYQLQFLDKYLYRPRGWENIDIKLN |
| | | EWSFVVEEAYDIEWDLNSKTPKLIPSPKSNRNKLYLAIPFTLKGNVKEPPLDKIVLKSETKKDHSRDKNRLNYP |
| | | ILGVDVGEYGVAWCLTKFDYNQDFSLRDIDIQGKGFIEDRNIGKIKDYFAEIQQKSRKGAYDEDDTTIAKVREN |
| | | AIGKLRNAIHSILTGSLEGASPVYEDAISNFETGSGKTIKIYNSVKRADTEFKSEADKAEHSLVWGKKDRNQET |
| | | KYIGRNVSAYASSYTCVNCLHTLFKVKKEDLSNIKILEKDGRIVTMSSPYGPDKKVRGYLSEKEKYEIGYQFKE |
| | | SEEDLKAFRKIVRDFARPPVNKNSEVLEKYAKEILAGNKIEEFRKKRGNSAIFVCPFCQFKADADIQAAFMMA |
| | | MRGYLRFSGIVPSKENSKNNPQESEDKSLKNSKKQSETGDTFLTKTAEYLQQLRFEIKEKIKEAVKVDF |
| 138 | 2237365 | VQGRAQYMDKKAKKIVGYKLHKNRIIFSGGNIIRTIKYPLVTDEKSKQFLEDFKAKVISDDRVVRGDVNLNDY |
| | | LNYSYQGNGFYTLFDFWLDSLRAGVVWAPKSVKLKEYSQVFFGLKSNLDELWNGVNDEIKRVFDKEKFLNN |
| | | FILKKEIRSTATRNSFQKKLKDSLIKDVKTDDEEVENLINSLVSKFFDSNGHIKLNGRDEQNKAWKCFGIDKEKI |
| | | FSKKADNQSRNSQLGDITFLIIPKLISFNKEENEEKIEDLVEKRRDWLKNLRIVKKQQEDYLNNILGISENFNGFS |
| | | NYFGSVLRFLKKGKTEEVVTALMSYLPQDDYFRNNIRNALEFLSQKAKKIPNVSMPKLVSGWADYRSVFGGK |
| | | IQSWFSNFNKRQSELEKKLDKFKKSLSDTKKFFLKDERIKSKEETKTAVIFIERLESFSNKFIKSEKGFKVFETLL |
| | | SALKKQLNLLYQQYLFDEKKEGDNVRKDRVLGALYQKIDKPIAFYAETQKSRNKKFIEDTISILEEGIDFLEKLI |
| | | RDFVGTFAVNYAFMGEKRKDETEESVYRKQLQFLWNKWKDAEINSSEFKNEYKKLLTSVLGEKEAKDIFNRK |
| | | NLGRYVFYKSKYAKGNVIEIKLSNGDEKNYLQEFDNLVKHLTKFLLSFSKDKLISNEKLLLDWLELGKNIISNL |
| | | IRFNQKTEFDLTSIKSLVKFEKARNYIECFKIEKIEKKEYGFIIQSLLLAELRGAATLYSKKSYIASYSVQVIGSDN |
| | | KFKLYYRPLENIGSLEEMLNKPKTERKDLVRIRHRYLISPFGFRKIGNKNFPENTLETIRLGKKKAEVETLFLND |
| | | LNSRAFYLQSSYYQLQFLDKYLYASKDWQNVDVELNEWSFAVEEAYRIKWNLDQKRPVFERDLKNRHNKLY |
| | | LAIPFSLKSRGERDLFLLNKMLKSQGEKDKQRLERDKNRLNYPILGVDVGEYGLAWALVKFNLDPDLQGMSI |
| | | LGRGFIEDKNVGKIKDYYVELQQRSRLGTYDSYDTTIVRVRENAIGKLRNYLHAVLLNLDEGGTVVYEDSISN |
| | | FETGSGKTVRIYNSVKRADTEFEAKADKSAHEQVWGKGTKYVGRALSAYASSYTCVNCCRSIYQFKAGDQD |
| | | KILAERKEGNIVFLKTPLGEALAYSKNKLSAKSGKNRFKEMMKILKDFARPPLEKSEVIRKYVFEKGIISEAKIN |
| | | SLKRKRGNSAVFVCPFCQFVADADIQAALMMAIRGYLRFSGLVSSLAKTEDKSLEVKDSLSETGNSFLKHTIFT |
| | | LEGIPAEDKTQIKETVKLONTILLAN |
| 139 | 2236971 | MAESKQMQCRKCGASMKYEVIGLGKKSCRYMCPDCGNHTSARKIQNKKKRDKKYGSASKAQSQRIAVAGA |
| | | LYPDKKVQTIKTYKYPADLNGEVHDSGVAEKIAQAIQEDEIGLLGPSSEYACWIASQKQSEPYSVVDFWFDAV |
| | | CAGGVFAYSGARLLSTVLQLSGEESVLRAALASSPFVDDINLAQAEKFLAVSRRTGQDKLGKRIGECFAEGRL |
| | | EALGIKDRMREFVQAIDVAQTAGQRFAAKLKIFGISQMPEAKQWNNDSGLTVCILPDYYVPEENRADQLVVL |
| | | LRRLREIAYCMGIEDEAGFEHLGIDPGALSNFSNGNPKRGFLGRLLNNDIIALANNMSAMTPYWEGRKGELIER |
| | | LAWLKHRAEGLYLKEPHFGNSWADHRSRIFSRIAGWLSGCAGKLKIAKDQISGVRTDLFLLKRLLDAVPQSAP |
| | | SPDFIASISALDRFLEAAESSQDPAEQVRALYAFHLNAPAVRSIANKAVQRSDSQEWLIKELDAVDHLEFNKAF |
| | | PFFSDTGKKKKKGANSNGAPSEEEYTETESIQQPEDAEQEVNGQEGNGASKNQKKFQRIPRFFGEGSRSEYRIL |
| | | TEAPQYFDMFCNNMRAIFMQLESQPRKAPRDFKCFLKNRLQKLYKQTFLNARSNKCRALLESVLISWGEFYT |
| | | YGANEKKFRLRHEASERSSDPDYVVQQALEIARRLFLFGFEWRDCSAGERVDLVEIHKKAISFLLAITQAEVSV |
| | | GSYNWLGNSTVSRYLSVAGTDTLYGTQLEEFLNATVLSQMRGLAIRLSSQELKDGFDVQLESSCQDNLQHLL |
| | | VYRASRDLAACKRATCPAELDPKILVLPAGAFIASVMKMIERGDEPLAGAYLRHRPHSFGWQIRVRGVAEVG |
| | | MDQGTALAFQKPTESEPFKIKPFSAQYGPVLWLNSSSYSQSQYLDGFLSQPKNWSMRVLPQAGSVRVEQRVA |
| | | LIWNLQAGKMRLERSGARAFFMPVPFSFRPSGSGDEAVLAPNRYLGLFPHSGGIEYAVVDVLDSAGFKILERG |
| | | TIAVNGFSQKRGERQEEAHREKQRRGISDIGRKKPVQAEVDAANELHRKYTDVATRLGCRIVVQWAPQPKPG |
| | | TAPTAQTVYARAVRTEAPRSGNQEDHARMKSSWGYTWSTYWEKRKPEDILGISTQVYWTGGIGESCPAVAV |
| | | ALLGHIRATSTQTEWEKEEVVFGRLKKFFPS |
| 140 | 2237255 | MTKDCKTGYKLHLQRLIFSGGEVTRSVKFPLKTIHDSSAPLKECFENKIIADDLKLRGGINLPDYQQNPNNYGL |
| | | AQFWIDSLRAGVIWQPTASKLTDFVRRYFKEPSKGDEIFAKAARVNTKLRFFEKETFVETIILKGPKRSGGSSG |
| | | GFRNELIGTVKMEYKEGKNKLSPQAQPVIDQILNDFYDQNGKLKLDANKQNEYWLKEFDIDKSLLEKEQSGA |
| | | ANKELAKLTFFIIPQLVNLDKELTPSQLLEQRKSLFLNPDADSLIEDGKLAELLGLKNNFNALSHYLNYAFQLL |
| | | YSDKTDEIFQAFTERAPTIAPQEKEVKEALEFLAHKAKKLGSPQTGRGWHEYRSVFGGKIQSWFSNYQRREKE |
| | | LEKQTQKLQQGIKNAQILIDERVRDDSLLKQEAQAVTDLLNQISHVLGKNTDNLQESKEYEVFLDLLAQARRE |
| | | LNFFYQKYLKEEDDETAINKHKELKYFYSAVYKPMTFFAQKRLEQNEKTLRKTIAIIEEGLQIAYRFLDDNQAN |
| | | FDPSSTFEKVKRKNETPEFLYRRSLQRIWGRLKDKRINSAEFRQKYSQILKKALEDESRWNKICADPNRYCFYK |
| | | SPYAKGTSTEIALKSAEFLAEFRSLVEDLYGFAVSLKDTSIFDNEGQLQDKNELLKTLLADLVRFSTKERFDIAG |
| | | MDLSQFEKAQKYLELLNRDKLDKNEAAYFYQSLIFSELRGAITVFTNKKYKSRYVLQLMRSPNKFPLFYYSRI |
| | | KALDAKALKLNRKDIRGQDLLSQNPHYYCIASSQKVEPAKKVQSSPFLRLLRKGTEIVGLEEADHKYLLKISTS |
| | | PYQLQFLERFVSPIGDWKKVDITLSEWSFIVEKNYAVDWNRKTKQPTFTQNGETKLFVSIPFQISAEKLKEEAG |
| | | LTNLSAEHLAHPILGIDVGEYGVGWVLAKFSDGIADILRRGFIRDRNIAAIQDNFASIQARAKKGVFNASSNTV |
| | | AQVRENAIGAVRNQIHDILTQETASVVYEYSISNFETGGGRVTRVYNSVKRADVYPETGADKLEHAHVWGDK |
| | | YANPGTHLSAYASSYTCASCARSIYKFNQKEIGQARIIAREGNILTIETPKGKVCGYGRDKKFREGYHFKNSKE |
| | | GFKEFMKLVKDFGRPPVSTRSETLSRFYAHLSKKQLDQFAQQRGNSAIFVCPFVDCHNVADADIQAAFMMAI |
| | | RGYLNWSEQVKTDEKEKKDFLGKTLEFLHSLKNRHQLSKEFSVSADVS |
| 141 | 2237413 | RTFKYPLVKTCKKHGKQKATNFLDVPHDLIEAIKHDDLYIRGPSNLVDYIKYKDAKIYTLIDFWLDSLRAGVV |
| | | FWPDNKSMQMDLMQINDFNLYEKFLHNAPMILRIFEPATFVKEILFLDIRGGNKKSKLREKLEKSVKNNINNN |
| | | RELALQAKELINKISGALIKGQNLLAPKDRKVYWLSEWNIDIDKIKNFFNLNITAIPHPDVTSFMENELTPENIISI |
| | | INSIIISHIEFAFRYPNLLPTTLKNIFEKIKIENKEFFTKDLKIKVSLNEILLKIKSLATNKTNKRGNSEEKSSRKIIED |
| | | LTDKYLKKTFGLSKKAAGLSNYLNKFLRYLKQQKTKKLLDYYKDKLGFNQKSQLKTIELRLIWLSKRADKFL |
| | | SAADKPKVFGVKGWHDYRQIINGKIASWLTNHIKRLSEQTEQLKNLIKSFNKIVEFINKYPKEEQKYNMQSNP |
| | | AHKENILKLIKQIRELAKNVSQVQENIKQYKDVASLVKALRQELNEYSQLYYRNQEGQKVKPSEISYLKDFWD |
| | | LRIYKPRSFYGYAQREKLEKIARSTIPTIKSGINLVQGLLKQLQNLPNYANAEKETDKNSIRKSAGFRRLLEALA |
| | | RKIRDHKINTKPFRDKYLQILQNYANSYELKNLNKYVFFKSNYSTSTQHEIELKNINYADEAKSVVTNLIEFLSS |
| | | FTQKDLLKDPQLLLDWVETAKLVIARMFKWMDQQKEWDLNNFNLEVFQATKDFIKSLKSNDVKTRMFRYVI |
| | | INLMFSELRGASIMFSRKLIKTKWNISFVDSLSKYKLMAISQKDNNKIQLSQNLPQRLKWYVIIPKKLAKSIKKL |
| | | EQPLLYFTKISTKKIKGENIVYERFIKLGKDKLGYRIIQVPNIINNMTLQICTSKYQLQFLERLVYQPKNWQNIIIH |
| | | ISNPSLIVEETHEISWEHNKNKITLSKIQTTNSPILYYALPLQLIVNRPNKKNTPLEAIKKRLHKIKTISNDNKRQK |
| | | AWYELNNDILYYLGADIGEYGIAWVITKINEKNNIISVEEYGFEFDNQIRKIQDKFNEIQQKARKGVFLEASTEL |
| | | AKLRENAIGYLRNKLHDKLIKYKGHIIYEANISGFESGVGRTTKIYDSVKRADIGNHPDVTKDIGKQLANHIWG |
| | | LKKGNKENSPGWQVSATGTSYTCINCGQTIYGLGREKDNWERTGTIAGNIHKFKHPIQNITILGYKENFKGQQI |
| | | DYKTLRKIAKNFARPPLVSIDKNTGKLKPSAVSEQFIINKDVLTEKQLEVVKKKRGNSAIFVCPFEGCHAVTDA |
| 142 | 2237262 | VHTPKVSGIKAFQVVFNSESIGDKDMNLVHSTPKVSGIKAIRHSGNSESIGDKGFPVERLLPSTLFRFYWYKVM |
| | | SKKLTGYTLHNKRLEYTGKSAIRSIKYPLKPPTQEELDSFSEKIVWDYEHLVGPLNISSYNKNPNMYSLVDFWI |
| | | DSLRAGVIWQSKSNLLINLVRKLERDILPTEKIFEQIDFKLKSKLDREKVIATIILNTGIRSLSTHKSLRNRFLKCF |
| | | KEEFWDNTDVLACVDRWSQALIVKGKSIPSKEQRLYWEKEFNIKISSFSEGVKLSPKFTFYINPSLEFNPNLSLD |
| | | KCLERVQNIEDNPESLLGLDNNFSAFSNYFNEVFAILYKGEVKNLVDTVVEMCPKWSDKKEALEERLKFLSRR |
| | | AKELGLPKLSNSWADYRMVIGGKLKSWFSNYRGQLEKIKEDLEEHREALKTLRKDIEKIEVIKDDPFTKTMKG |
| | | DLTEQRVVLLKLLETLLVGDKIAIDDFELYQFLLADFKGLLNTAYQKSIQTEKEKKEDKDVSKKYNALYKKLH |
| | | NMSRFFGDAKKERYQKIVNSSLPAIDIGLKILADILEKLETTKEQDFSLITEEYVKKQLEKLNKKYKSRAFNYN |
| | | KFNQITEQILKKYNNGNLPKFNEVFYKHPREKSINNKELPIEISNPPETFRYLIDKYKIDLDWKTLNPNEVADFV |
| | | EVYKFTLGWLLTLNKSLKVDFSRYDMPLFPKASAFIKREGGELEGYSLNKVIFSFIASEIKGFVTLYTRETFVVR |
| | | YVIQTIASEKKFPLLCIQEDKKEETQDFSRKWGVLIRGEEPKRESKIKCLKIVGKKNVTSAKGEYVSPDFIWHIK |
| | | TSKYQIQFLNRLFCKTKKWNSMDIELSEPCLILEEKWKILWDKEKMKPVFEKEENDQKVYYSMPFDLKPVAN |
| | | QKQRKQRAEEIENRTAYLGLDVGEFGVAYVAMKVDDNKIKILEKGFITDPALRKIRTHIQSFKERQKAATFAN |
| | | PSTKIARVREMAIHSLRNQIHNLALKYNAKIIYEISISNFETGGNKIAKIYRSLKVSDVYPENGADRAVVKAVW |
| | | GERSKLMGNHISSYATSYTCIKCFRTPFELDLDKKYSFSKEGGKVVVDINNTKVSGFVRKSTDNDSKSNKLEV |
| | | KGKVALGWIKDYARPPISEVLLPESNLEQKLHNRGNSYIYRCPFCGYVTDADIQAAFNIACRGYISEIKKGKKG |
| | | KGFEYIKEVKTLWSKNNNPWKEFEFPI |

TABLE 2 provides illustrative sequences of exemplary heterologous polypeptide modifications of effector protein(s) that are useful in the compositions, systems and methods described herein.

TABLE 2
SEQUENCES OF EXEMPLARY HETEROLOGOUS
POLYPEPTIDE MODIFICATIONS OF EFFECTOR PROTEIN(S)

| SEQ | | |
|---|---|---|
| ID NO: | Description | Protein or Sequence* |
| 29 | NLS | KRPAATKKAGQAKKKK |
| 30 | NLS | PKKKRKV |
| 31 | NLS | LPPLERLTL |
| 145 | EEP | GLFXALLXLLXSLWXLLLXA |
| 146 | NLS | KR(K/R)R |
| 147 | NLS | (P/R)XXKR(^{∧}D/E)(K/R) |
| 148 | NLS | KRX(W/F/Y)XXAF |
| 149 | NLS | (R/P)XXKR(K/R)(^{∧}D/E) |
| 150 | NLS | LGKR(K/R)(W/F/Y) |
| 151 | NLS | KRX₁₀K(K/R)(K/R) |
| 152 | NLS | K(K/R)RK |
| 153 | NLS | KRX₁₁K(K/R)(K/R) |
| 154 | NLS | KRX₁₂K(K/R)(K/R) |
| 155 | NLS | KRX₁₀K(K/R)X(K/R) |
| 156 | NLS | KRX₁₁K(K/R)X(K/R) |
| 157 | NLS | KRX₁₂K(K/R)X(K/R) |
| 158 | NLS | APKKKRKVGIHGVPAA |

*wherein X is any naturally occurring amino acid; and
^{∧}D/E is any naturally occurring amino acid except Asp or Glu

TABLE 3 provides illustrative PAM sequences that are useful in the compositions, systems and methods described herein.

TABLE 1
EXEMPLARY PAM SEQUENCES

| Effector Protein | |
|---|---|
| SEQ ID NO: | PAM Sequence* (5′ → 3′) |
| — | NNN |
| — | NNNN |
| — | NNNNN |
| — | NNNNNN |
| — | NNNNNNN |
| 1-28 | YTTN |
| 1-28 | TTYN |
| 1-28 | TTTN |
| 2, 8-9 | TTCN |
| 2 | CTTN |

*wherein each N is independently any one of A, G, C, or T; wherein Y is C or T

TABLE 4 provides illustrative repeat sequences for use in guide nucleic acids that are useful in the compositions, systems and methods described herein.

TABLE 4
EXEMPLARY REPEAT SEQUENCES
FOR USE IN GUIDE NUCLEIC ACIDS

| Effector | Repeat Sequence | Repeat |
|---|---|---|
| Protein | (5′ → 3′), | Sequence |
| SEQ ID NO: | shown as RNA | SEQ ID NO: |
| 1-28 | AGAUUUCUACUUUUGUAGAU | 74 |
| 1-28 | AGAUUUCUACUAUUUGUAGAU | 75 |
| 1-28 | AGAUUUCUACUGUUGUAGAU | 76 |
| 1-28 | AGAUUUCUACUUUCGUAGAU | 77 |
| 1-28 | UAAUUUCUACUAAGUGUAGAU | 78 |
| 1-28 | AGAUUUCUACUUUUGUAGAU | 74 |
| 1-28 | GAAUUUCUACUAUUGUAGAU | 79 |

TABLE 5 provides illustrative crRNA sequences that are useful in the compositions, systems and methods described herein, wherein bold font denotes the repeat sequence and underline denotes the spacer sequence.

TABLE 5
EXEMPLARY CRRNA SEQUENCES
FOR USE IN SINGLE GUIDE SYSTEMS

| Effector | crRNA Sequence | crRNA |
|---|---|---|
| Protein | (5′ → 3′), | Sequence |
| SEQ ID NO: | shown as RNA | SEQ ID NO: |
| 1-28 | AGAUUUCUACUUUUGUAGAUU | 34 |
| | AUUAAAUACUCGUAUUGCU | |
| 6 | UAAUUUCUACUAAGUGUAGAU | 163 |
| | AGCUGCUCGAAUUGGCUUUG | |
| 3 and 5 | GAAUUUCUACUAUUGUAGAUA | 164 |
| | AGCCAAUUCGAGCAGCUGA | |

TABLE 6 provides illustrative sgRNA sequences that are useful in the compositions, systems and methods described herein.

TABLE 6
EXEMPLARY SGRNA SEQUENCES FOR USE IN SINGLE GUIDE SYSTEMS

| Effector | | sgRNA |
|---|---|---|
| Protein | | Sequence |
| SEQ ID NO. | sgRNA Sequence (5′ → 3′), shown as RNA | SEQ ID NO: |
| 1-28 | AGAUUUCUACUUUUGUAGAUUAUUAAAUACUCGUAUUGCUGUUCGAUUAU | 57 |
| 1-28 | AGAUUUCUACUUUUGUAGAUUAUUAAAUACUCGUAUUGCUGUUCG | 58 |
| 1-28 | AGAUUUCUACUUUUGUAGAUUAUUAAAUACUCGUAUUGCUGU | 59 |
| 1-28 | AGAUUUCUACUUUUGUAGAUUAUUAAAUACUCGUAUUGCUG | 60 |
| 1-28 | AGAUUUCUACUUUUGUAGAUUAUUAAAUACUCGUAUUGCU | 34 |
| 1-28 | AGAUUUCUACUUUUGUAGAUUAUUAAAUACUCGUAUUGC | 61 |
| 1-28 | AGAUUUCUACUUUUGUAGAUUAUUAAAUACUCGUAUUG | 62 |
| 1-28 | AGAUUUCUACUUUUGUAGAUUAUUAAAUACUCGUAUU | 63 |
| 1-28 | AGAUUUCUACUUUUGUAGAUUAUUAAAUACUCGUAU | 64 |
| 1-28 | AGAUUUCUACUUUUGUAGAUCACAGCUUGUCUGUAAGCGGAUGCCAUAUG | 65 |
| 1-28 | AGAUUUCUACUUUUGUAGAUCACAGCUUGUCUGUAAGCGGAUGCC | 66 |
| 1-28 | AGAUUUCUACUUUUGUAGAUCACAGCUUGUCUGUAAGCGGAU | 67 |
| 1-28 | AGAUUUCUACUUUUGUAGAUCACAGCUUGUCUGUAAGCGGA | 68 |
| 1-28 | AGAUUUCUACUUUUGUAGAUCACAGCUUGUCUGUAAGCGG | 69 |
| 1-28 | AGAUUUCUACUUUUGUAGAUCACAGCUUGUCUGUAAGCG | 70 |
| 1-28 | AGAUUUCUACUUUUGUAGAUCACAGCUUGUCUGUAAGC | 71 |
| 1-28 | AGAUUUCUACUUUUGUAGAUCACAGCUUGUCUGUAAG | 72 |
| 1-28 | AGAUUUCUACUUUUGUAGAUCACAGCUUGUCUGUAA | 73 |
| 1-28 | AGAUUUCUACUAUUUGUAGAUUAUUAAAUACUCGUAUUGCU | 80 |
| 1-28 | AGAUUUCUACUGUUGUAGAUUAUUAAAUACUCGUAUUGCU | 81 |
| 1-28 | AGAUUUCUACUUUCGUAGAUUAUUAAAUACUCGUAUUGCU | 82 |
| 1-28 | UAAUUUCUACUAAGUGUAGAUUAUUAAAUACUCGUAUUGCU | 83 |
| 1-28 | UAUUAAAUACUCGUAUUGCU | 43 |

TABLE 7 provides illustrative target nucleic acids that are useful in the compositions, systems and methods described herein.

TABLE 7
EXEMPLARY TARGET NUCLEIC ACIDS
Exemplary targets

AAVS1, ABCA4, ABCB11, ABCC8, ABCD1, ABCG5, ABCG8, ACAD9, ACADM, ACADVL, ACAT1, ACOX1, ACSF3, ADA,
ADAMTS2, ADGRG1, AGA, AGL, AGPS, AGXT, AHI1, AIRE, ALDH3A2, ALDOB, ALG6, ALK, ALKBH5, ALMS1, ALPL, AMRC9,
AMT, ANAPC10, ANAPC11, ANGPTL3, APC, Apo(a), APOCIII, APOEε4, APOL1, APP, AQP2, AR, ARFRP1, ARG1, ARH,
ARL13B, ARL6, ARSA, ARSB, ASL, ASNS, ASPA, ASS1, ATM, ATP6V1B1, ATP7A, ATP7B, ATRX, ATXN1, ATXN10, ATXN2,
ATXN3, ATXN7, ATXN8OS, AXIN1, AXIN2, B2M, BACE-1, BAK1, BAP1, BARD1, BAX2, BBS1, BBS10, BBS12, BBS2, BCKDHA,
BCKDHB, BCL2L2, BCS1L, BEST1, Betaglobin gene, BLM, BMPR1A, BRAF, BRAFV600E, BRCA1, BRCA2, BRIP1, BSND,
C9orf72, C282Y, CA4, CACNA1A, CAH1, CAPN3, CASR, CBS, CCNB1 CC2D2A, CCR5, CD1, CD2, CD3, CD3D, CD3Z, CD4,
CD5, CD6, CD7, CD8A, CD8B, CD9, CD14, CD18, CD19, CD21, CD22, CD23, CD27, CD28, CD30, CD33, CD34, CD36, CD38,
CD40, CD40L, CD44, CD46, CD47, CD48, CD52, CD55, CD57, CD58, CD59, CD68, CD69, CD72, CD73, CD74, CD79A, CD80,
CD81, CD83, CD84, CD86, CD90, CD93, CD96, CD99, CD100, CD123, CD160, CD163, CD164, CD164L2, CD166, CD200,
CD204, CD207, CD209, CD226, CD244, CD247, CD274, CD276, CD300, CD320, CDC73, CDH1, CDH23, CDK11, CDK4,
CDKN1A, CDKN1B, CDKN1C, CDKN2A, CDKN2B, CEBPA, CELA3B, CEP290, CERKL, CFB, CFTR, CHCHD10, CHEK2, CHM,
CHRNE, CIITA, CLN3, CLN5, CLN6, CLN8, CLRN1, CLTA, CMT1A, CNBP, CNGB1, CNGB3, COL1A1, COL1A2, COL27A1,
COL4A3, COL4A4, COL4A5, COL7A1, CPS1, CPT1A, CPT2, CRB1, CREBBP, CRX, CRYAA, CINNA1, CTNNB1, CTNND2,
CTNS, CTSK, CXCL12, CYBA, CYBB, CYP11B1, CYP11B2, CYP17A1, CYP19A1, CYP27A1, DBT, DCC, DCLRE1C, DERL2,
DFNA36, DFNB31, DGAT2, DHCR7, DHDDS, DICER1, DIS3L2, DLD, DMD, DMPK, DNAH5, DNAI1, DNAI2, DNM2, DNMT1,
DPC4, DUX4, DYSF, EDA, EDN3, EDNRB, EGFR, EIF2B5, EMC2, EMC3, EMD, EMX1, EN1, EPCAM, ERCC6, ERCC8, ESCO2,
ETFA, ETFDH, ETHE1, EVC, EVC2, EYS, F5, F9, FX1, FAH, FAM161A, FANCA, FANCB, FANCC, FANCD1, FANCD2, FANCE,
FANCF, FANCG, FANCI, FANCJ, FANCL, FANCM, FANCN, FANCP, FANCS, FBN1, FGF14, FGFR2, FGFR3, FGA, FGB, FGG,
FH, FHL1, FIX, FKRP, FKTN, FLCN, FMR1, FOXP3, FSCN2, FSHD1, FUS, FUT8, FVIII, FXI, FXII, FXN, G6PC, GAA, GALC,
GALK1, GALT, GAMT, GATA2, GATA-4, GBA, GBE1, GCDH, GCGR, GDNF, GFAP, GFM1, GHR, GJB1, GJB2, GLA, GLB1,
GLDC, GLE1, GNE, GNPTAB, GNPTG, GNS, GPC3, GPR98, GREM1, GRHPR, GRIN2B, H2AFX, H2AX, HADHA, HAX1, HBA1,
HBA2, HBB, HER2, HEXA, HEXB, HFE, HGSNAT, HLCS, HMGCL, HOGA1, HOXB13, HPRPF3, HPRT1, HPS1, HPS3, HRAS,
HRD1, HSD17B4, HSD3B2, HTT, HUS1, HYAL1, HYLS1, IDS, IDUA, IFITM5, IKBKAP, IL2RG, IL7R, IMPDH1, INPP5E, IRF4,
ITGB2, ITPR1, IVD, JAG1, JAK1, JAK3, KCNC3, KCND3, KCNJ11, KLHL7, KRAS, LAMA2, LAMA3, LAMB3, LAMC2, LCA5,
LDLR, LDLRAP 1, LHX3, LIFR, LIPA, LMNA, LOR, LOXHD1, LPL, LRAT, LRP6, LRPPRC, LRRK2, MADR2, MAN2B1, MAPT,
MAX, MCM6, MCOLN1, MECP2, MED17, MEFV, MEN1, MERTK, MESP2, MET, METex14, MFN2, MFSD8, MIA3, MITF, MKL2,
MKS1, MLC1, MLH1, MLH3, MMAA, MMAB, MMACHC, MMADHC, MMD, MPI, MPL, MPV17, MSH2, MSH3, MSH6,
MTHFD1L, MTHFR, MTM1, MTRR, MTTP, MUT, MUTYH, MYC, MYH7, MYO7A, NAGLU, NAGS, NBN, NDRG1, NDUFAF5,
NDUFS6, NEB, NF1, NF2, NKX2-5, NOG, NOTCH1, NOTCH2, NPC1, NPC2, NPHP1, NPHS1, NPHS2, NRAS, NR2E3, NTHL1,
NTRK, NTRK1, OAT, OCT4, OFD1, OPA3, OTC, PAH, PALB2, PAQR8, PAX3, PC, PCCA, PCCB, PCDH15, PCSK9, PD1,
PDCD1, PDE6B, PDGFRA, PDHA1, PDHB, PEX1, PEX10, PEX12, PEX13, PEX14, PEX16, PEX19, PEX2, PEX26, PEX3, PEX5,
PEX6, PEX7, PFKM, PHGDH, PHOX2B, PKD1, PKD2, PKHD1, PKK, PLEKHG4, PMM2, PMP22, PMS1, PMS2, PNPLA3,
POLD1, POLE, POMGNT1, POT1, POU5F1, PPM1A, PPP2R2B, PPT1, PRCD, PRKAG2, PRKAR1A, PRKCG, PRNP, PROM1,
PROP1, PRPF31, PRPF8, PRPH2, PRPS1, PSAP, PSD95, PSEN1, PSEN2, PSRC1, PTCH1, PTEN, PTS, PUS1, PYGM, RAB23,
RAD50, RAD51C, RAD51D, RAG1, RAG2, RAPSN, RARS2, RB1, RDH12, RECQL4, RET, RHO, RICTOR, RMRP, ROS1, RP1, RP2,
RPE65, RPGR, RPGRIP1L, RPL32P3, RS1, RTCA, RTEL1, RUNX1, SACS, SAMHD1, SCN1A, SCN2A, SDHA, SDHAF2, SDHB,
SDHC, SDHD, SEL1L, SEPSECS, SERPINA1, SERPINC1, SERPING1, SGCA, SGCB, SGCG, SGSH, SIRT1, SLC12A3, SLC12A6,
SLC17A5, SLC22A5, SLC25A13, SLC25A15, SLC26A2, SLC26A4, SLC35A3, SLC35B4 SLC37A4, SLC39A4, SLC4A11, SLC6A8,
SLC7A7, SMAD3, SMAD4, SMARCA4, SMARCAL1, SMARCB1, SMARCE1, SMN1, SMPD1, SNAI2, SNCA, SNRNP200, SOD1,
SOX10, SPARA7, SPTBN2, STAR, STAT3, STK11, SUFU, SUMF1, SYNE1, SYNE2, SYS1, TARDBP, TAT, TBK1, TBP, TCIRG1,
TCTN3, TECPR2, TERC, TERT, TFR2, TGFBR2, TGM1, TH, TLE3, TMEM127, TMEM138, TMEM216, TMEM43, TMEM67,
TMPRSS6, TOP1, TOPORS, TP53, TPP1, TRAC, TRMU, TSC1, TSC2, TSFM, TSPAN14, TTBK2, TTC8, TTPA, TTR, TULP1,
TYMP, UBE2G2, UBE2J1, UBE3A, USH1C, USH1G, USH2A, VEGF, VHL, VPS13A, VPS13B, VPS35, VPS45, VRK1, VSX2, VWF,
WAS, WDR19, WDR48, WFS1, WNT10A, WRN, WS2B, WS2C, WT1, XPA, XPC, XPF, XRCC3, YAP1, ZAC1, ZEB1, ZFYVE26, and
ZNF423

TABLE 8 provides illustrative diseases and syndromes for compositions, systems and methods described herein.

TABLE 8
DISEASES AND SYNDROMES
Exemplary Diseases and Syndromes

11-hydroxylase deficiency; 17,20-desmolase deficiency; 17-hydroxylase deficiency; 3-hydroxyisobutyrate aciduria; 3-
hydroxysteroid dehydrogenase deficiency; 46,XY gonadal dysgenesis; AAA syndrome; ABCA3 deficiency; ABCC8-associated
hyperinsulinism; aceruloplasminemia; acromegaly; achondrogenesis type 2; acral peeling skin syndrome; acrodermatitis
enteropathica; adrenocortical micronodular hyperplasia; adrenoleukodystrophies; adrenomyeloneuropathies; Aicardi-Goutieres
syndrome; Alagille disease (also called Alagille Syndrome); Alexander Disease, Alpers syndrome; alpha-1 antitrypsin deficiency
(AATD); alpha-mannosidosis; Alstrom syndrome; Alzheimer's disease; amebic dysentery; amelogenesis imperfecta; amish type
microcephaly; amyotrophic lateral sclerosis (ALS); anaplastic large cell lymphoma; anauxetic dysplasia; androgen insensitivity
syndrome; angiopathic thrombosis; antiphospholipid syndrome; Antley-Bixler syndrome; APECED, Apert syndrome, aplasia of
lacrimal and salivary glands, arginase-1 deficiency; argininosuccinic aciduria; argininemia; arrhythmogenic right ventricular
dysplasia; Arts syndrome; ARVD2; arylsulfatase deficiency type metachromatic leukodystrophy; ataxia telangiectasia;
atherosclerotic cardiovascular disease; autoimmune lymphoproliferative syndrome; autoimmune polyglandular syndrome type 1;
autosomal dominant anhidrotic ectodermal dysplasia; autosomal dominant deafness; autosomal dominant polycystic kidney disease;
autosomal recessive microtia; autosomal recessive renal glucosuria; autosomal visceral heterotaxy; babesiosis; balantidial
dysentery; Bardet-Biedl syndrome; Bartter syndrome; basal cell nevus syndrome; Batten disease; benign recurrent intrahepatic
cholestasis; beta-mannosidosis; β-thalassemia; Bethlem myopathy; Blackfan-Diamond anemia; Bleeding disorder (coagulation);
blepharophimosis; Byler disease; C syndrome; CADASIL; Calcific Aortic Stenosis; calcification of joints and arteries; carbamoyl
phosphate synthetase deficiency; cardiofaciocutaneous syndrome; Carney triad; carnitine palmitoyltransferase deficiencies;
cartilage-hair hypoplasia; cblC type of combined methylmalonic aciduria; CD18 deficiency; CD3Z-associated primary T-cell
immunodeficiency; CD40L deficiency; CDAGS syndrome; CDG1A; CDG1B; CDG1M; CDG2C; CEDNIK syndrome; central core
disease; centronuclear myopathy; cerebral capillary malformation; cerebrooculofacioskeletal syndrome type 4;
cerebrooculogacioskeletal syndrome; cerebrotendinous xanthomatosis; Chaga's Disease; Charcot Marie Tooth Disease; cherubism;
CHILD syndrome; chronic granulomatous disease; chronic recurrent multifocal osteomyelitis; cirrhosis; citrin deficiency;
citrullinemia type I; citrullinemia type II; classic hemochromatosis; CNPPB syndrome; cobalamin C disease; Cockayne syndrome;
coenzyme Q10 deficiency; Coffin-Lowry syndrome; Cohen syndrome; combined deficiency of coagulation factors V; common
variable immune deficiency 3; complement hyperactivation; complete androgen insentivity; cone rod dystrophies; conformational
diseases; congenital adrenal hyperplasia; congenital bile adid synthesis defect type 1; congenital bile adid synthesis defect type 2;
congenital defect in bile acid synthesis type; congenital erythropoietic porphyria; congenital generalized osteosclerosis; congenital
hyperplasia (CAH); Cornelia de Lange syndrome; coronary heart disease; Cousin syndrome; Cowden disease; COX deficiency; Cri
du chat syndrome; Crigler-Najjar disease; Crigler-Najjar syndrome type 1; Crisponi syndrome; Crouzon syndrome; Currarino
syndrome; Curth-Macklin type ichthyosis hystrix; cutis laxa; cystic fibrosis; cystinosis; d-2-hydroxyglutaric aciduria; DDP
syndrome; Dejerine-Sottas disease; Denys-Drash syndrome; Dercum disease; desmin cardiomyopathy; desmin myopathy; DGUOK-
associated mitochondrial DNA depletion; diabetes Type I; diabetes Type II; disorders of glutamate metabolism; distal spinal
muscular atrophy type 5; DNA repair diseases; dominant optic atrophy; Doyne honeycomb retinal dystrophy; Dravet Syndrome;
Duchenne muscular dystrophy; dyskeratosis congenita; Ehlers-Danlos syndrome type 4; Ehlers-Danlos syndromes; Elejalde disease;
Ellis-van Creveld disease; Emery-Dreifuss muscular dystrophies; encephalomyopathic mtDNA depletion syndrome; encephalitis;
enzymatic diseases; EPCAM-associated congenital tufting enteropathy; epidermolysis bullosa with pyloric atresia; epilepsy; fabry
disease; facioscapulohumeral muscular dystrophy; Factor V Leiden Thrombophilia; Faisalabad histiocytosis; familial atypical
mycobacteriosis; familial capillary malformation-arteriovenous; Familial Creutzfeld-Jakob Disease; familial esophageal achalasia;
familial glomuvenous malformation; familial hemophagocytic lymphohistiocytosis; familial mediterranean fever; familial
megacalyces; familial schwannomatosis; familial spina bifida; familial splenic asplenia/hypoplasia; familial thrombotic
thrombocytopenia purpura; Fanconi disease (Fanconi anemia); Feingold syndrome; FENIB; fibrodysplasia ossificans progressiva;
FKTN; Fragile X syndrome; Francois-Neetens fleck corneal dystrophy; Frasier syndrome; Friedreich's ataxia; FTDP-17; Fuchs
Corneal Dystrophy; fucosidosis; G6PD deficiency; galactosialidosis; Galloway syndrome; Gardner syndrome; Gaucher disease;
Gitelman syndrome; GLUT1 deficiency; GM2- Gangliosidoses (e.g., Tay Sachs Disease, Sandhoff Disease) glycogen storage
disease type 1b; glycogen storage disease type 2; glycogen storage disease type 3; glycogen storage disease type 4; glycogen storage
disease type 9a; glycogen storage diseases; GM1-gangliosidosis; Greenberg syndrome; Greig cephalopolysyndactyly syndrome;
hair genetic diseases; hairy cell leukemia; HANAC syndrome; harlequin type ichtyosis congenita; HDR syndrome; hearing loss;
hemochromatosis type 3; hemochromatosis type 4; hemolytic anemia; hemolytic uremic syndrome; hemophilia A; hemophilia B;
hereditary angioedema type 3; hereditary angioedemas; hereditary hemorrhagic telangiectasia; hereditary hypofibrinogenemia;
hereditary intraosseous vascular malformation; hereditary leiomyomatosis and renal cell cancer; hereditary neuralgic amyotrophy;
hereditary sensory and autonomic neuropathy type; Hermansky-Pudlak disease; HHH syndrome; HHT2; hidrotic ectodermal
dysplasia type 1; hidrotic ectodermal dysplasias; histiocytic sarcoma; HNF4A-associated hyperinsulinism; HNPCC; homozygous
familial hypercholesterolemia; human immunodeficiency with microcephaly; Human monkeypox (MPX); human papilloma virus
(HPV) infection; Huntington's disease; hyper-IgD syndrome; hyperinsulinism-hyperammonemia syndrome; hypercholesterolemia;
hypertrophy of the retinal pigment epithelium; hypochondrogenesis; hypohidrotic ectodermal dysplasia; ICF syndrome; idiopathic
congenital intestinal pseudo-obstruction; immunodeficiency 13; immunodeficiency 17; immunodeficiency 25; immunodeficiency
with hyper-IgM type 1; immunodeficiency with hyper-IgM type 3; immunodeficiency with hyper-IgM type 4; immunodeficiency
with hyper-IgM type 5; immunoglobulin alpha deficiency; inborn errors of thyroid metabolism; infantile myofibromatosis; infantile
visceral myopathy; infantile X-linked spinal muscular atrophy; intrahepatic cholestasis of pregnancy; IPEX syndrome; IRAK4
deficiency; isolated congenital asplenia; Jeune syndrome; Johanson-Blizzard syndrome; Joubert syndrome; JP-HHT syndrome;
juvenile hemochromatosis; juvenile hyalin fibromatosis; juvenile nephronophthisis; Kabuki mask syndrome; Kallmann syndromes;
Kartagener syndrome; KCNJ11-associated hyperinsulinism; Kearns-Sayre syndrome; Kostmann disease; Kozlowski type of
spondylometaphyseal dysplasia; Krabbe disease; LADD syndrome; late infantile-onset neuronal ceroid lipofuscinosis; LCK
deficiency; LDHCP syndrome; Leber Congenital Amaurosis Type 10; Legius syndrome; Leigh syndrome; lethal congenital
contracture syndrome 2; lethal congenital contracture syndromes; lethal contractural syndrome type 3; lethal neonatal CPT
deficiency type 2; lethal osteosclerotic bone dysplasia; leukocyte adhesion deficiency; Li Fraumeni syndrome; LIG4 syndrome;
lipodystrophy; lissencephaly type 1; lissencephaly type 3; Loeys-Dietz syndrome; low phospholipid-associated cholelithiasis; Lynch
Syndrome; lysinuric protein intolerance; a lysosomal storage disease (e.g., Hunter syndrome, Hurler syndrome); macular dystrophy;
Maffucci syndrome; Majeed syndrome; mannose-binding protein deficiency; mantle cell lymphoma; Marfan disease; Marshall
syndrome; MASA syndrome; mastocytosis; MCAD deficiency; McCune-Albright syndrome; MCKD2; Meckel syndrome; MECP2
Duplication Syndrome; Meesmann corneal dystrophy; megacystis-microcolon-intestinal hypoperistalsis; megaloblastic anemia type
1; MEHMO; MELAS; Melnick-Needles syndrome; MEN2s; meningitis; Menkes disease; metachromatic leukodystrophies;
methymalonic acidemia due to transcobalamin receptor defect; methylmalonic acidurias; methylvalonic aciduria; microcoria-
congenital nephrosis syndrome; microvillous atrophy; migraine; mitochondrial neurogastrointestinal encephalomyopathy;
monilethrix; monosomy X; mosaic trisomy 9 syndrome; Mowat-Wilson syndrome; mucolipidosis type 2; mucolipidosis type Ma;
mucolipidosis type IV; mucopolysaccharidoses; mucopolysaccharidosis type 3A; mucopolysaccharidosis type 3C;
mucopolysaccharidosis type 4B; multiminicore disease; multiple acyl-CoA dehydrogenation deficiency; multiple cutaneous and
mucosal venous malformations; multiple endocrine neoplasia type 1; multiple sulfatase deficiency; mycosis fungoides; myotonic
dystrophy; NAIC; nail-patella syndrome; nemaline myopathies; neonatal diabetes mellitus; neonatal surfactant deficiency;
nephronophtisis; Netherton disease; neurofibromatoses; neurofibromatosis type 1; Niemann-Pick disease type A; Niemann-Pick
disease type B; Niemann-Pick disease type C; NKX2E; non-alcoholic fatty liver disease (NAFLD); non-alcoholic steatohepatitis
(NASH); Noonan syndrome; North American Indian childhood cirrhosis; NROB1 duplication-associated DSD; ocular genetic
diseases; oculo-auricular syndrome; OLEDAID; oligomeganephronia; oligomeganephronic renal hypolasia; Ollier disease; Opitz-
Kaveggia syndrome; ornithine transcarbamylase deficiency (OTCD); orofaciodigital syndrome type 1; orofaciodigital syndrome
type 2; osseous Paget disease; osteogenesis imperfecta; otopalatodigital syndrome type 2; OXPHOS diseases; palmoplantar
hyperkeratosis; panlobar nephroblastomatosis; Parkes-Weber syndrome; Parkinson's disease; partial deletion of 21q22.2-q22.3;
Pearson syndrome; Pelizaeus-Merzbacher disease; Pendred syndrome; pentalogy of Cantrell; peroxisomal acyl-CoA-oxidase
deficiency; Peutz-Jeghers syndrome; Pfeiffer syndrome; Pierson syndrome; pigmented nodular adrenocortical disease; pipecolic
acidemia; Pitt-Hopkins syndrome; plasmalogens deficiency; platelet glycoprotein IV deficiency; pleuropulmonary blastoma and
cystic nephroma; polycystic kidney disease; polycystic ovarian disease; polycystic lipomembranous osteodysplasia; Pompe disease,
including infantile onset Pompe Disease (IOPD) and late onset Pompe Disease (LOPD); porphyrias; PRKAG2 cardiac syndrome,
premature ovarian failure; primary erythermalgia; primary hemochromatoses; primary hyperoxaluria; progressive familial
intrahepatic cholestasis; propionic acidemia; protein-losing enteropathy; pyruvate decarboxylase deficiency; RAPADILINO
syndrome; renal cystinosis; retinitis pigmentosa; Rett Syndrome; rhabdoid tumor predisposition syndrome; Rieger syndrome; ring
chromosome 4; Roberts syndrome; Robinow-Sorauf syndrome; Rothmund-Thomson syndrome; severe combined
immunodeficiency disorder (SCID); Saethre-Chotzen syndrome; Sandhoff disease; SC phocomelia syndrome; SCAS; Schinzel
phocomelia syndrome; short rib-polydactyly syndrome type 1; severe hypertriglyceridemia; short rib-polydactyly syndrome type 4;
short-rib polydactyly syndrome type 2; short-rib polydactyly syndrome type 3; Shwachman disease; Shwachman-Diamond disease;
sickle cell anemia; Silver-Russell syndrome; Simpson-Golabi-Behmel syndrome; Smith-Lemli-Opitz syndrome; SPG7-associated
hereditary spastic paraplegia; spherocytosis; spinocerebellar ataxia; spinal muscular atrophy; split-hand/foot malformation with long
bone deficiencies; spondylocostal dysostosis; sporadic amyotrophic lateral sclerosis; sporadic visceral myopathy with inclusion
bodies; storage diseases; Stargardt macular dystrophy; STRA6-associated syndrome; stroke; Tay-Sachs disease; thanatophoric
dysplasia; thrombophilia due to antithrombin III deficiency; thyroid metabolism diseases; Tourette syndrome; transcarbamylase
deficiency; transthyretin-associated amyloidosis; trisomy 13; trisomy 22; trisomy 2p syndrome; tuberous sclerosis; tufting
enteropathy; urea cycle diseases; Usher Syndrome; Van Den Ende-Gupta syndrome; Van der Woude syndrome; variegated mosaic
aneuploidy syndrome; VLCAD deficiency; von Hippel-Lindau disease; von Willebrand disease; Waardenburg syndrome; WAGR
syndrome; Walker-Warburg syndrome; Werner syndrome; Wilson disease; Wiskott-Aldrich Syndrome; Wolcott-Rallison
syndrome; Wolfram syndrome; X-linked agammaglobulinemia; X-linked chronic idiopathic intestinal pseudo-obstruction; X-linked
cleft palate with ankyloglossia; X-linked dominant chondrodysplasia punctata; X-linked ectodermal dysplasia; X-linked Emery-
Dreifuss muscular dystrophy; X-linked lissencephaly; X-linked lymphoproliferative disease; X-linked visceral heterotaxy;
xanthinuria type 1; xanthinuria type 2; xeroderma pigmentosum; XPV; and Zellweger disease

### EXAMPLES

The following examples are included for illustrative purposes only and are not intended to limit the scope of the invention.

### Example 1. Effector Proteins Cleave DNA at High Temperatures

Some Cas effectors are more resistant to higher temperatures (>50° C.) in in vitro applications where high temp is desirable. A screen was carried out to identify heat-resistant trans cleavage nucleases.

Varying concentration of purified effector proteins represented by SEQ ID NOs: 1-28 were tested in a trans cleavage assay for trans cleavage activity. The target nucleic acid was double stranded DNA containing 20 bp randomized PAM protospacers, and the reporter nucleic acid was single stranded DNA. The assay was performed at 37° C. in a buffer of 20 mM Tricine pH 9 at 37° C., 15 mM Mg(OAc)2, 1 mM TCEP, and 0.2 mg/mL BSA.

The target nucleic acid contained a protospacer sequence having a spacer sequence of TATTAAATACTCGTATTGCT (SEQ ID NO: 32) with an additional seven nucleotides flanking each side of the spacer sequence. This target duplex is designed to be amenable to cleavage by any effector proteins regardless of its PAM preference. The entire sequence of the sense strand of the target nucleic acid was: GGTGGTAATGCCATGTAATATGCCTTCGAANNNNNNNGCAGGCATACACTGAAG TTCNNNNNNNCTACAAACTCTTCCTGTTAGTTAGCNNNNNNNAACTTGACACTTA ATGCTTGNNNNNNNTGCAAGGGGTGTTATGAGCCATCCTNNNNNNNCCAGCAGT TTGGCCAGCCACNNNNNNNTTGACCTTTGTTACTACTCTAGCCANNNNNNNTATT AAATACTCGTATTGCTNNNNNNNCCCAGTCACGACGTTGTAAAACGGANNNNN NNCACAGCTTGTCTGTAAGCGGNNNNNNNGTACTAGCCTGTGTGAAATTGTTATC CGCT (SEQ ID NO: 33), with bold letters indicating the protospacer sequence.

A single guide RNA, a crRNA, containing a repeat sequence and spacer sequence were used in the reaction at a concentration of 50 nM. The crRNA sequence was the sequence of SEQ ID NO: 34, with lower case letter denoting the repeat sequence and uppercase letters denoting the spacer sequence.

The nucleic acid reporter was used in the reaction at a concentration of 200 nM. The T12 reporter nucleic acid was referred to as/56-FAM/TTTTTTTTTTTT/3IABkFQ/(SEQ ID NO: 35), wherein the sequence of the reporter nucleic acid is TTTTTTTTTTTT (SEQ ID NO: 35), the 56-FAM represents a fluorescein, and the 3IABkFQ represents a fluorescein quencher. Detectable signal from the reporter was recorded every minute for 10 minutes. trans cleavage activity is indicated by a fluorescent signal that is released upon cleavage of this reporter. Fluorescence was quantified and compared between samples to determine relative trans cleavage activity among samples.

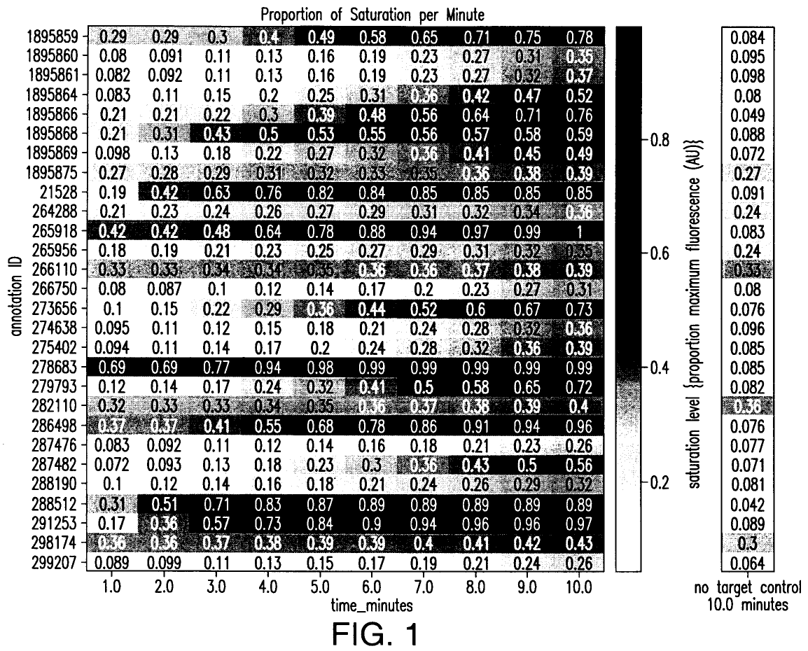

Results of the trans cleavage assay are shown in FIG. 1. A heat map of the first ten minutes of the time courses of active effector proteins was plotted as the fold of saturation (saturation representing the digestion of the entire 200 nM reporter pool) for each effector protein. A non-target control shows the background level of non-target dependent (E. coli background) nuclease activity at the longest time point. In general, these effector proteins had low background activity, with target dependent activity representing that of the effector proteins being easily identifiable. All effector proteins provided measurable trans cleavage activity in this assay. Values are normalized to the saturation point of digestion of 200 nM T12 reporter.

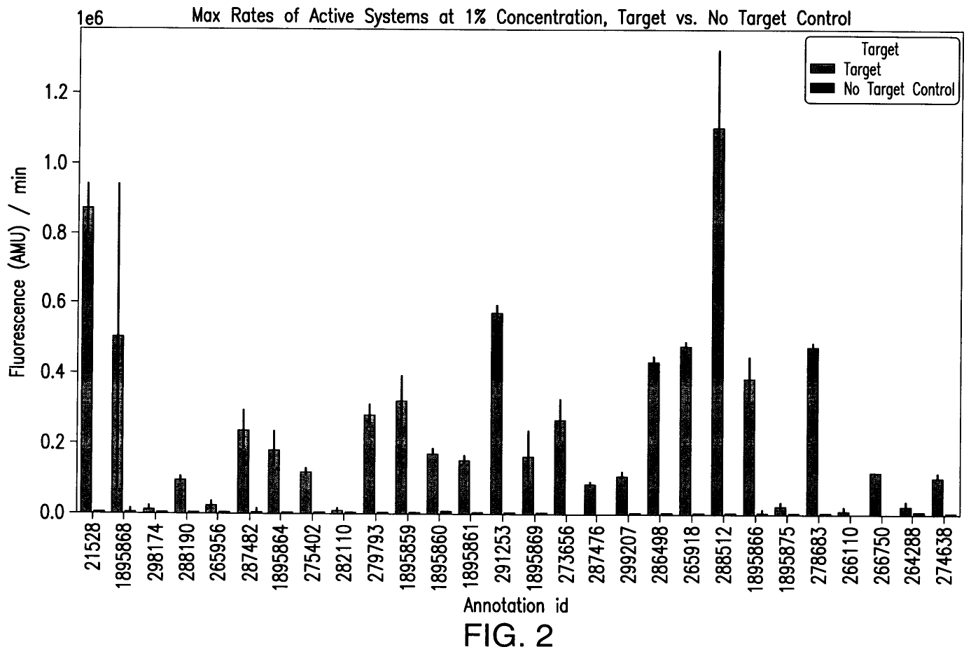

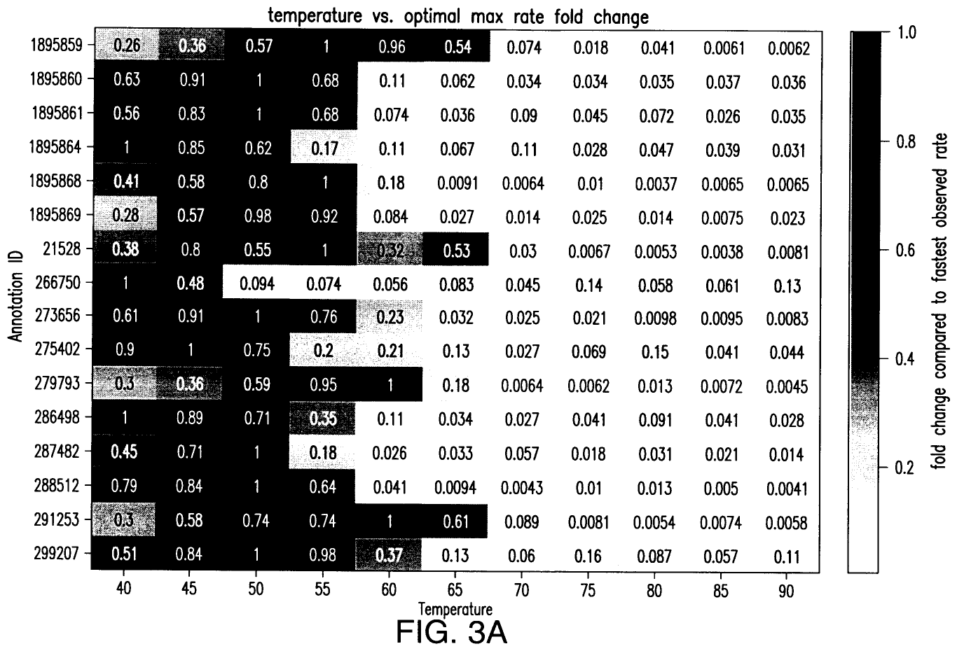

FIG. 2 shows the maximum rate of each effector protein in this assay. Max rates (the four points in the time course with the highest slope) were heat map plotted.

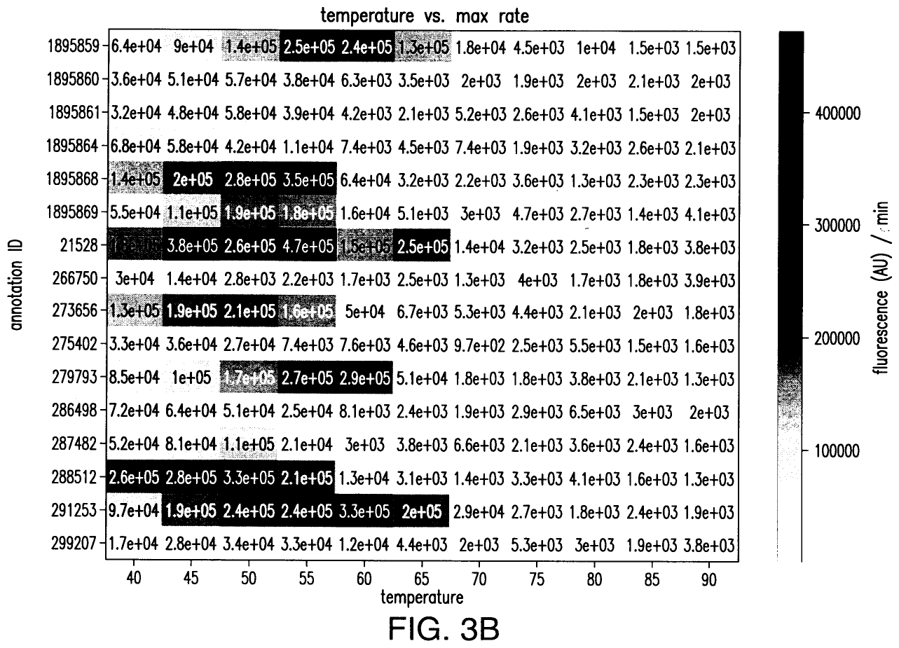

Select effector proteins that exhibited trans cleavage activity at 37° C. were similarly tested at temperatures ranging from 40° C. to 90° C. FIG. 3A shows normalized heat map data where the peak max rate is the maximum value (=1) and the activity at other temperatures is relative to this max rate. FIG. 3B shows relative, non-normalized heat map data. Optimal temperature for trans cleavage activity varied among the effector proteins. Proteins 21528, 1895859, 279793 and 291253 demonstrated notable trans cleavage activity at 65° C.

### Example 2. Effector Proteins Cleave DNA at High Salt Concentrations

Nuclease activity is sensitive to the salt concentration in the reaction. Some Cas effectors are more resistant to added salt in in vitro applications. A screen was carried out to identify salt-resistant trans cleavage nucleases. Effector proteins were tested in a trans cleavage assay for trans cleavage activity at varying salt concentrations. The target nucleic acid was double stranded DNA having a randomized PAM, and the reporter nucleic acid was single stranded DNA. The assay was performed at 37° C. in a buffer of 20 mM Tricine pH 9, 15 mM Mg(OAc)2, 1 mM TCEP, 0.2 mg/mL BSA, and varying concentrations of potassium acetate or sodium chloride.

The target nucleic acid contained a protospacer sequence having a spacer sequence of TATTAAATACTCGTATTGCT (SEQ ID NO: 32) with an additional seven nucleotides flanking each side of the spacer sequence. This target duplex is designed to be amenable to cleavage by any effector proteins regardless of its PAM preference. The entire sequence of the sense strand of the target nucleic acid was:

(SEQ ID NO: 33)
GGTGGTAATGCCATGTAATATGCCTTCGAANNNNNNNGCAGGCATA
CACTGAAGTTCNNNNNNNCTACAAACTCTTCCTGTTAGTTAGCNNN
NNNNAACTTGACACTTAATGCTTGNNNNNNNTGCAAGGGGTGTTAT
GAGCCATCCTNNNNNNNCCAGCAGTTTGGCCAGCCACNNNNNNNTT
GACCTTTGTTACTACTCTAGCCANNNNNNNTATTAAATACTCGTAT
TGCTNNNNNNNCCCAGTCACGACGTTGTAAAACGGANNNNNNNCAC
AGCTTGTCTGTAAGCGGNNNNNNNGTACTAGCCTGTGTGAAATTGT
TATCCGCT.

A single guide RNA, a crRNA, containing a repeat sequence and spacer sequence were used in the reaction at a concentration of 50 nM. The crRNA sequence was the sequence of SEQ ID NO: 34, with lower case letter denoting the repeat sequence and uppercase letters denoting the spacer sequence.

The nucleic acid reporter was used in the reaction at a concentration of 200 nM. The reporter nucleic acid was referred to as/56-FAM/TTTTTTTTTTTT/3IABkFQ/(SEQ ID NO: 35), wherein the sequence of the reporter nucleic acid is TTTTTTTTTTTT (SEQ ID NO: 35), the 56-FAM represents a fluorescein, and the 3IABkFQ represents a fluorescein quencher. Detectable signal from the reporter was measured every minute for 10 minutes. trans cleavage activity is indicated by a fluorescent signal that is released upon cleavage of this reporter. Fluorescence was quantified and compared between samples to determine relative trans cleavage activity among samples.

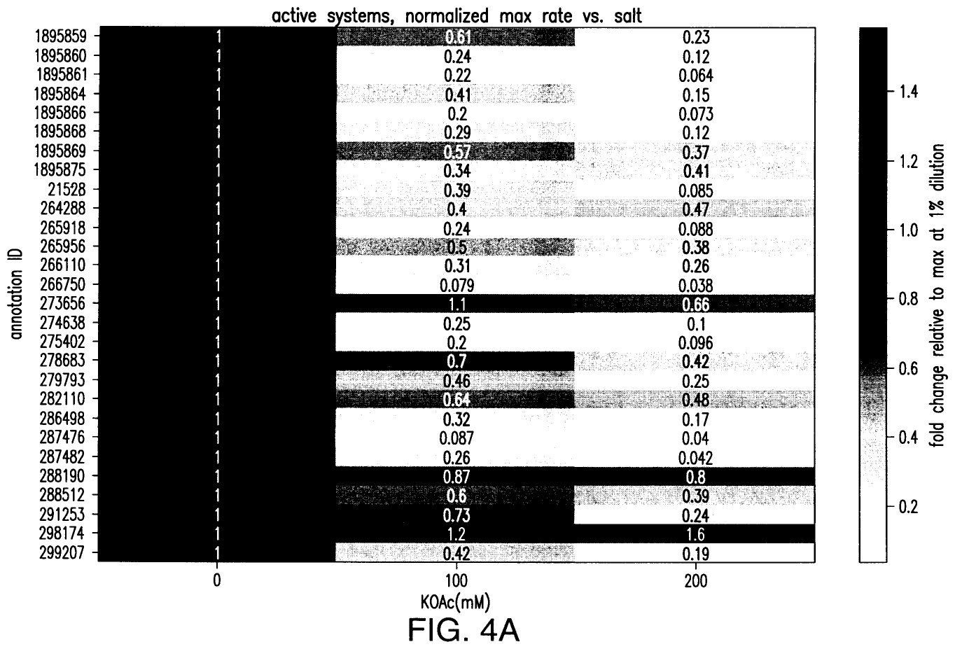

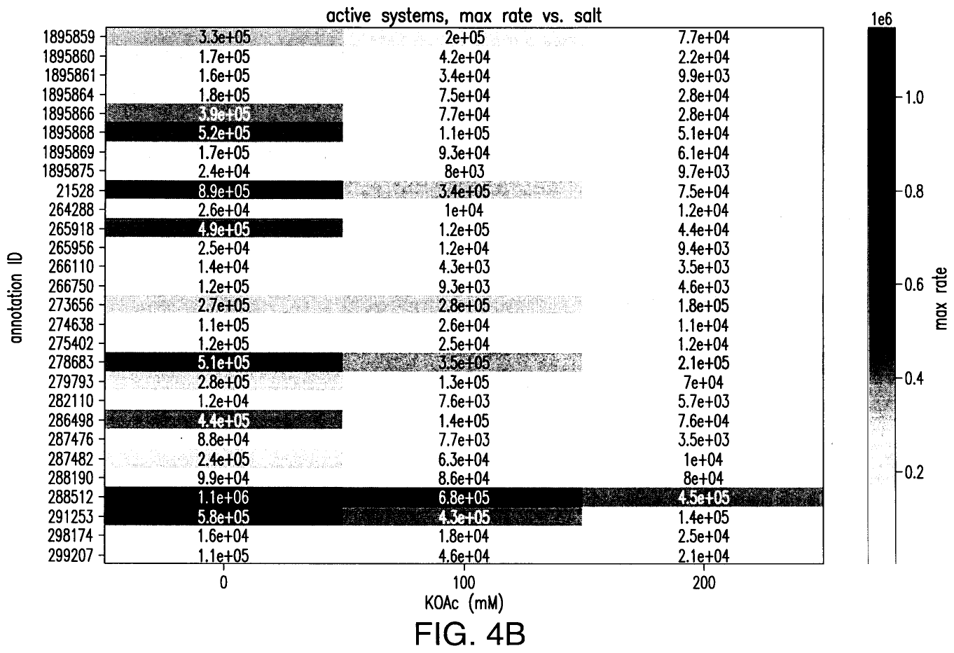

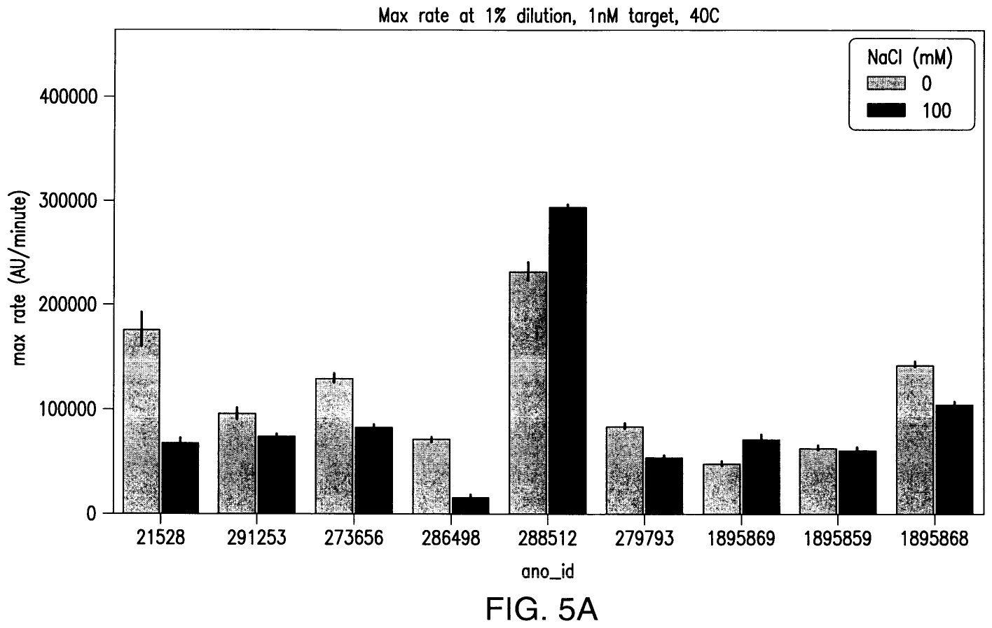

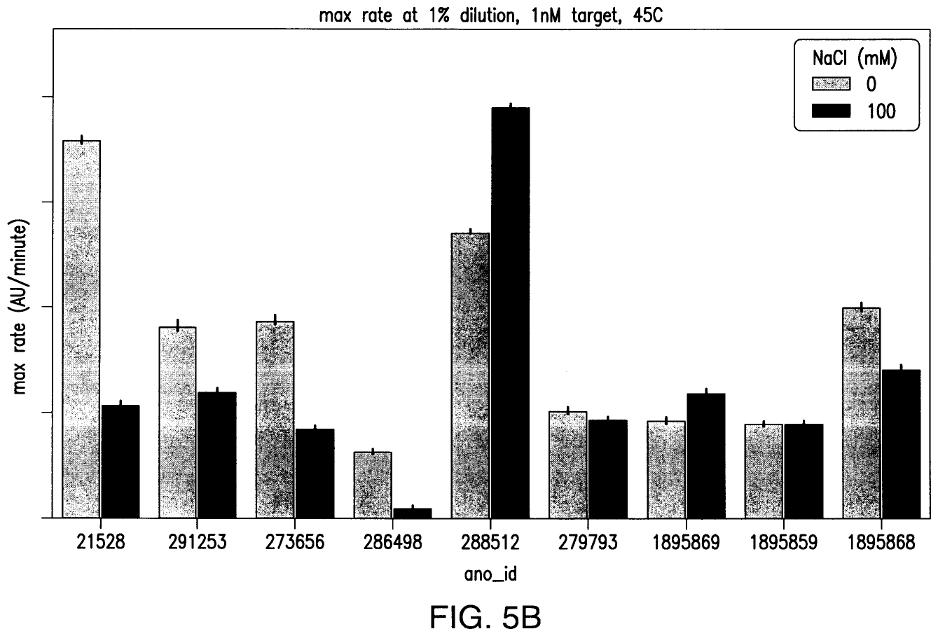

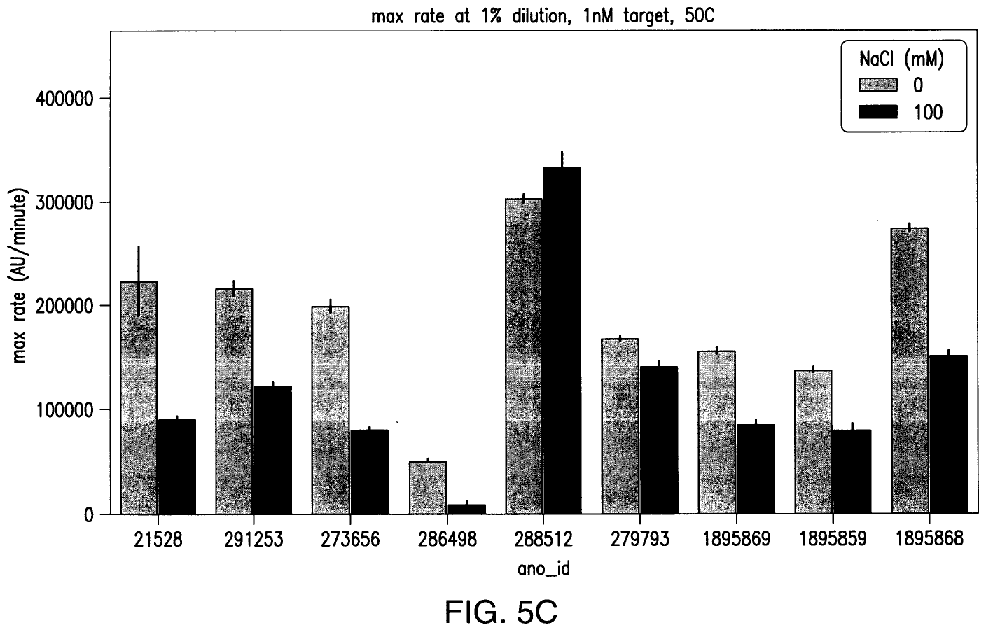

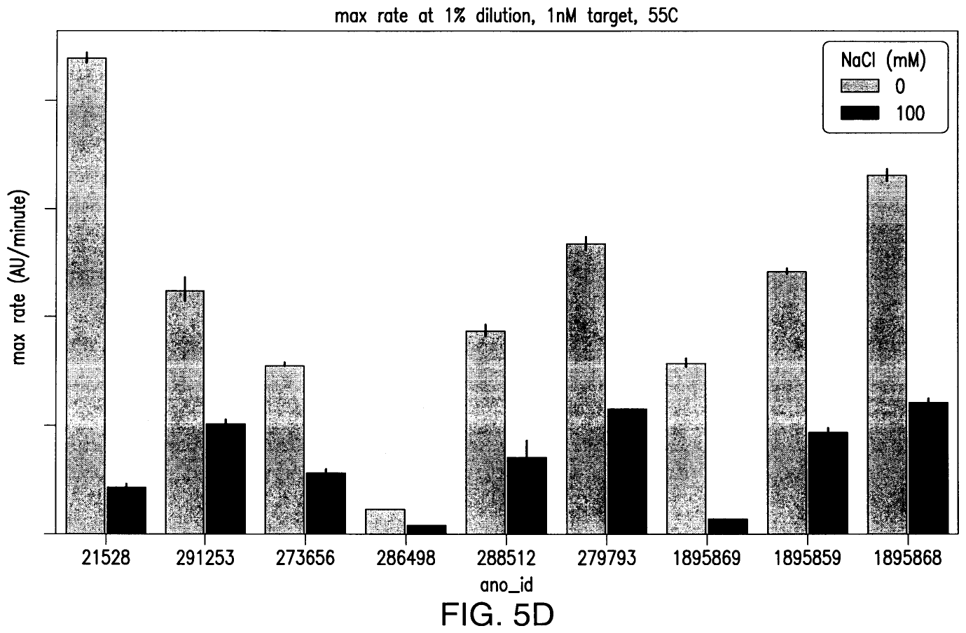

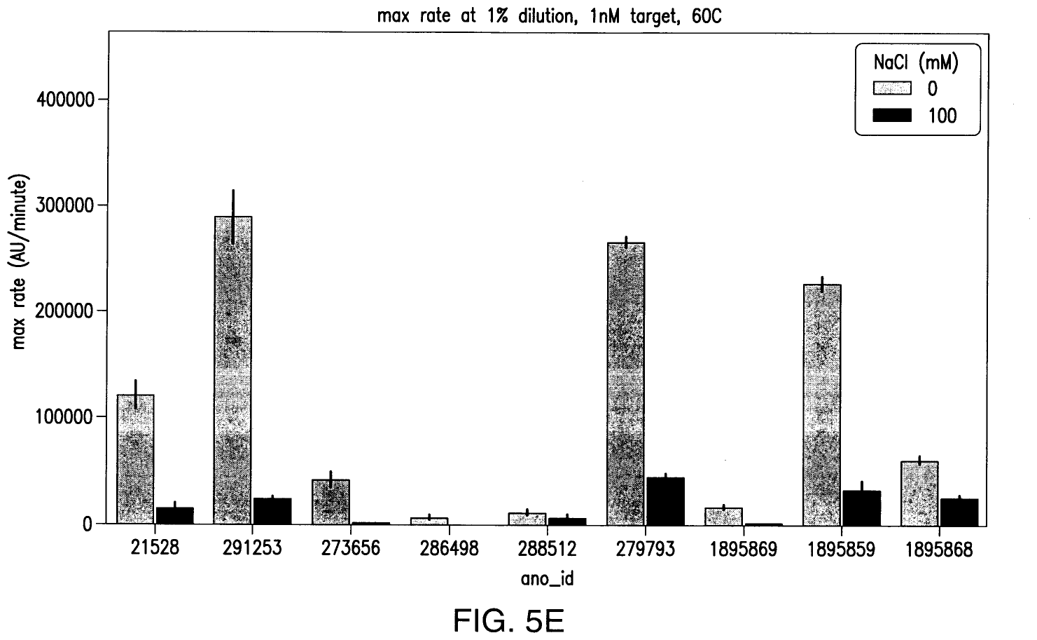

The results of these assays are shown in FIGS. 4A, 4B, 5A, 5B, 5C, 5D, and 5E.

In general, FIGS. 4A and 4B shows salt titration profiles of various effector proteins. In particular, FIG. 4A shows normalized heat map data where the no salt maximum rate values for each system are normalized to 1 and the activity at other potassium acetate concentrations is relative to this rate. Moreover, FIG. 4B shows a heat map of the corresponding non-normalized max rate values. Effector proteins 1895859, 1895869, 265956, 273656, 278683, 282110, 288190, 288512, 291253, and 298174 remained more than 50% active in the presence of salt when compared to no-salt controls.

In general, FIGS. 5A, 5B, 5C, 5D, and 5E show the maximum rate of trans cleavage activity of tested effector proteins in this assay in the presence of 100 mM sodium chloride at varying temperatures. FIGS. 5A, 5B, 5C, 5D, and 5E shows maximum rate of trans cleavage activity in the presence of 100 mM sodium chloride at 40° C., 45° C., 50° C., 55° C., and 60° C., respectively. Notably, effector proteins 21528, 291253, 273656, 286498, 288512, 279793, 1895869, 1895859, and 1895868 remained active in 100 mM sodium chloride at 55° C., and effector proteins 21528, 291253, 288512, 279793, 1895859, and 1895868 remained active in 100 mM sodium chloride at 60° C.

### Example 3. Effector Proteins Edit Genomic DNA in Mammalian Cells

Effector proteins are tested for their ability to produce indels in a mammalian cell line (e.g., HEK293T cells). Briefly, a plasmid encoding the effector proteins and a guide RNA are delivered by lipofection to the mammalian cells. This is performed with a variety of guide RNAs targeting several loci adjacent to biochemically determined PAM sequences. Indels in the loci are detected by next generation sequencing of PCR amplicons at the targeted loci and indel percentage is calculated as the fraction of sequencing reads containing insertions or deletions relative to an unedited reference sequence. “No plasmid” and Cas9 are included as negative and positive controls, respectively.

### Example 4. Base Editing

A nucleic acid vector encoding a fusion protein is constructed for base editing. The fusion protein comprises a catalytically inactive variant of an effector protein fused to a deaminase. The fusion protein and at least one guide nucleic acid is tested for its ability to edit a target sequence in eukaryotic cells. Cells are transfected with the nucleic acid vector and guide nucleic acid. After sufficient incubation, DNA is extracted from the transfected cells. Target sequences are PCR amplified and sequenced by NGS and MiSeq. The presence of base modifications is analyzed from sequencing data. Results are recorded as a change in % base call relative to the negative control.

### Example 5. Activation of Gene Expression with Cas Effector Fusion Polypeptide

A single stranded reporter nucleic acid encoding a fluorescent protein (e.g., enhanced green fluorescent protein (EGFP)) and a eukaryotic promoter is generated with a target sequence that is known to be recognized by complexes of effector proteins disclosed herein and corresponding guide nucleic acids. A nucleic acid vector encoding the Cas effector fused to a transcriptional activator; a guide nucleic acid; and the single stranded reporter nucleic acid encoding EGFP are introduced to eukaryotic cells via lipofection and EGFP expression is quantified by flow cytometry. Relative amounts of RNA, indicative of relative gene expression, are quantified with RT-qPCR.

### Example 6. Reduction of Gene Expression with Cas Effector Fusion Polypeptide

A single stranded reporter nucleic acid encoding a fluorescent protein (e.g., enhanced green fluorescent protein (EGFP)) and a pSV40 promoter that drives constitutive expression of EGFP is generated with a target sequence that is known to be recognized by complexes of effector proteins disclosed herein and corresponding guide nucleic acids. A nucleic acid vector encoding the Cas effector fused to a transcriptional repressor; a guide nucleic acid; and the single stranded reporter nucleic acid encoding EGFP are introduced to eukaryotic cells via lipofection and EGFP expression is quantified by flow cytometry. Relative amounts of RNA, indicative of relative gene expression, are quantified with RT-qPCR.

### Example 7. Generating a Catalytically Inactive Variant of a CRISPR Cas Effector Protein

Extensive work has been done to evaluate the overall domain structure of the CRISPR Cas enzymes in the last decade. These data can be an effective reference when trying to identify a catalytic residue of a Cas nuclease. By selecting the residue of a Cas nuclease of interest that aligns at the same relative location as the catalytic residue of a known nuclease when the Cas nuclease and known nuclease are aligned for maximal sequence identity, one can identify the catalytic residue of the Cas nuclease.

Sequence or structural analogs of a Cas nuclease provide an additional or supplemental way to predict the catalytic residues of the novel Cas nuclease relative to the previous description in this Example. Catalytic residues are usually highly conserved and can be identified in this manner.

Alternatively, or additionally to the description already provided in this Example, computational software may be used to predict the structure of a Cas nuclease.

### Example 8. PAM Screening for Effector Proteins

To assess the PAM preferences of exemplary effector proteins described herein, an in vitro enrichment (IVE) experiment assaying for nucleic acid cleaving activity was carried out.

Effector proteins (PROTEIN ID NOS: 273656, 286498, 279793, 288512, 1895859, 1895868, 291253, 265918, 1895869, and 1895866 were screened by an IVE assay to determine PAM recognition by each effector protein. Briefly, 50 micromolars of purified effector proteins was combined with 1,000 ng of a plasmid library containing a randomized PAM sequence 5′ of a target protospacer (5′-NNNNNNN-3′, where N is any of A, C, G, T) in 10× Cutsmart buffer and were carried out for 1, 5, and 60 minutes at 37° C. Reactions were terminated with 1 μl of proteinase K and 5 μl of 500 mM EDTA for 30 minutes at 37° C. Any target plasmid that was successfully cleaved had an adapter ligated to the cut end, enabling PCR amplification. After magnetic bead purification and MiSeq sequencing, next generation sequencing was performed on cut sequences to identify enriched PAMs.

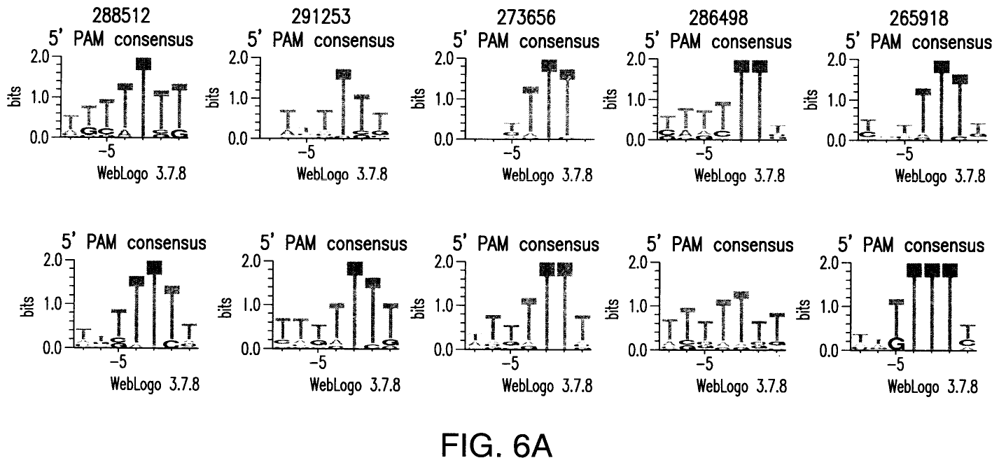

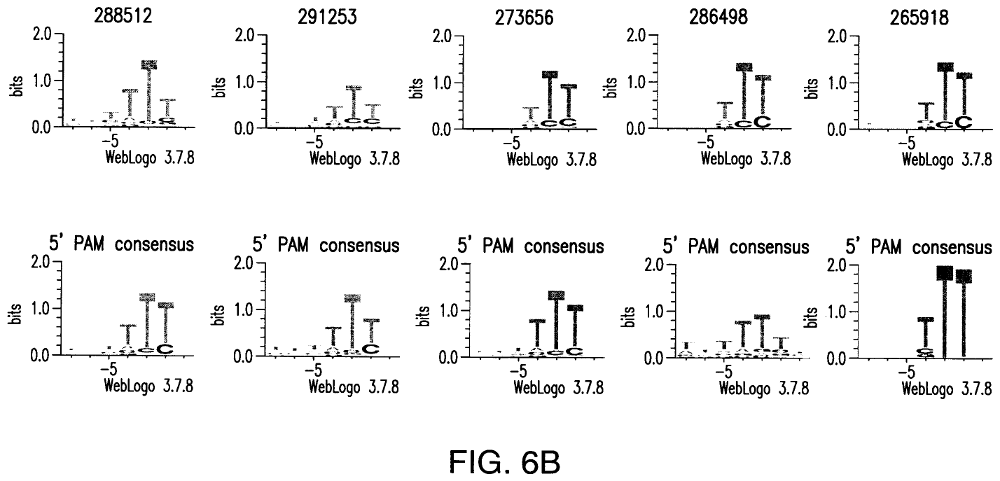

Results collected at the 1-, 5-, and 60-minute timepoints show that each complex had the same preference at each time point (data not shown). Results collected under high stringency conditions (using a 1% cutoff) can be seen in FIG. 6A. Results collected under low stringency conditions (using a 10% cutoff) can be seen in FIG. 6B. The results show that all candidates display a 5′ TTTN preference. PROTEIN ID Nos: 1895869 and 2695918 prefer TTTN and TTCN; 286498 prefers CTTN, TTTN, and TTCN.

### Example 9. Effector Proteins Spacer Length Titration

This example describes experiments performed to test preferred spacer lengths for exemplary effector proteins (PROTEIN ID NOS: 273656, 286498, 279793, 288512, 1895859, 1895868, 291253, 265918, 1895869, and 1895866).

The assay included 18 different guide RNAs, which were used with each protein; 9 featured spacer 1 (SP1 or S1) and 9 featured spacer 2 (SP2 or S2); all featured a 5′-AGAUUUCUACUUUUGUAGAU-3′ (SEQ ID NO: 74) repeat sequence. All guide RNAs targeted a 5′-TTTG-3′ PAM. The spacers tested ranged in length from 16 to 30 nucleotides. Sequences assayed can be seen in TABLE 9.

TABLE 9
Spacer Length Titration

| Spacer | Length | Spacer sequence | sgRNA sequence |
|---|---|---|---|
| SP1 | 30 | UAUUAAAUACUCGUAUUGCUGUU | (SEQ ID NO: 57) |
| | | CGAUUAU (SEQ ID NO: 39) | |
| SP1 | 25 | UAUUAAAUACUCGUAUUGCUGUU | (SEQ ID NO: 58) |
| | | CG (SEQ ID NO: 40) | |
| SP1 | 22 | UAUUAAAUACUCGUAUUGCUGU | (SEQ ID NO: 59) |
| | | (SEQ ID NO: 41) | |
| SP1 | 21 | UAUUAAAUACUCGUAUUGCUG | (SEQ ID NO: 60) |
| | | (SEQ ID NO: 42) | |
| SP1 | 20 | UAUUAAAUACUCGUAUUGCU | (SEQ ID NO: 34) |
| | | (SEQ ID NO: 43) | |
| SP1 | 19 | UAUUAAAUACUCGUAUUGC | (SEQ ID NO: 61) |
| | | (SEQ ID NO: 44) | |
| SP1 | 18 | UAUUAAAUACUCGUAUUG | (SEQ ID NO: 62) |
| | | (SEQ ID NO: 45) | |
| SP1 | 17 | UAUUAAAUACUCGUAUU | (SEQ ID NO: 63) |
| | | (SEQ ID NO: 46) | |
| SP1 | 16 | UAUUAAAUACUCGUAU | (SEQ ID NO: 64) |
| | | (SEQ ID NO: 47) | |
| SP2 | 30 | CACAGCUUGUCUGUAAGCGGAUG | (SEQ ID NO: 65) |
| | | CCAUAUG (SEQ ID NO: 48) | |
| SP2 | 25 | CACAGCUUGUCUGUAAGCGGAUG | (SEQ ID NO: 66) |
| | | CC (SEQ ID NO: 49) | |
| SP2 | 22 | CACAGCUUGUCUGUAAGCGGAU | (SEQ ID NO: 67) |
| | | (SEQ ID NO: 50) | |
| SP2 | 21 | CACAGCUUGUCUGUAAGCGGA | (SEQ ID NO: 68) |
| | | (SEQ ID NO: 51) | |
| SP2 | 20 | CACAGCUUGUCUGUAAGCGG | (SEQ ID NO: 69) |
| | | (SEQ ID NO: 52) | |
| SP2 | 19 | CACAGCUUGUCUGUAAGCG | (SEQ ID NO: 70) |
| | | (SEQ ID NO: 53) | |
| SP2 | 18 | CACAGCUUGUCUGUAAGC | (SEQ ID NO: 71) |
| | | (SEQ ID NO: 54) | |
| SP2 | 17 | CACAGCUUGUCUGUAAG | (SEQ ID NO: 72) |
| | | (SEQ ID NO: 55) | |
| SP2 | 16 | CACAGCUUGUCUGUAA | (SEQ ID NO: 73) |
| | | (SEQ ID NO: 56) | |

Effector proteins were incubated with guide RNA in a 1:1 ratio to form RNPs (ribonucleoproteins) at a final concentration of 50 nM and allowed to incubate for 20 minutes at room temperature (at 37° C.), followed by addition of appropriate target nucleic acid at a final concentration of 0.1 nM. Cleavage activity was detected by fluorescence signal produced upon cleavage of a fluorophore-quencher reporter (included in the assay at 200 nM).

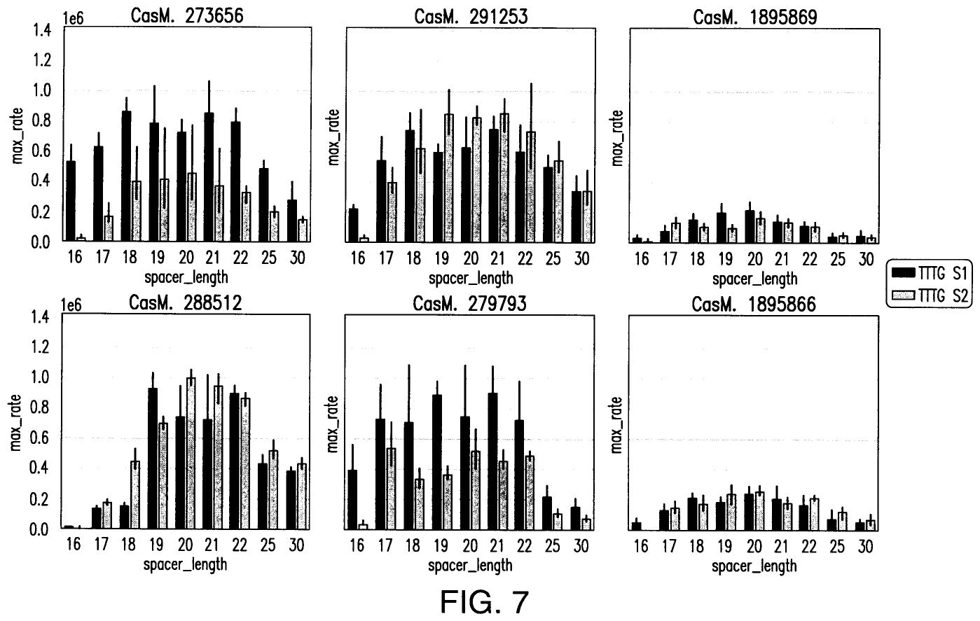

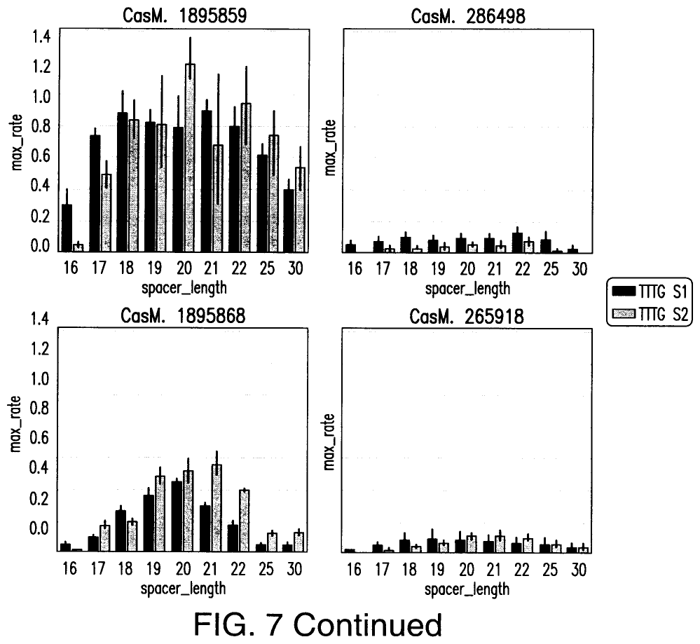

Results can be seen in FIG. 7. Top performers generally preferred spacers of length 20+/−3 bp, regardless of target.

### Example 10. Effector Proteins Repeat Comparison

This example describes experiments performed to test preferred repeats for exemplary effector proteins (PROTEIN ID NOS: 273656, 286498, 279793, 288512, 1895859, 1895868, 291253, 265918, 1895869, and 1895866).

The assay included 6 different (partial, 20 nt) repeats, which were tested with each exemplary effector protein. All sequences featured the same spacer sequence, 20 bp of S1, (SEQ ID NO: 43) and all guide RNAs targeted a 5′-TTTG-3′ S1 target. Sequences assayed can be seen in TABLE 10.

TABLE 10
Repeat Comparison

| | Repeat | Repeat | sgRNA |
|---|---|---|---|
| RNA No. | Name | Sequence | Sequence |
| R7898 | UAUUU | (SEQ ID NO: 75) | (SEQ ID NO: 80) |
| UAUUU-20 | | | |
| SP1 | | | |
| R7899 | UGUU | (SEQ ID NO: 76) | (SEQ ID NO: 81) |
| UGUU-20 | | | |
| SP1 | | | |
| R7900 | UUUC | (SEQ ID NO: 77) | (SEQ ID NO: 82) |
| UUUC-20 | | | |
| SP1 | | | |
| M08-20 | M08 | (SEQ ID NO: 78) | (SEQ ID NO: 83) |
| SP1 | | | |
| R4797-20 | R4797 | (SEQ ID NO: 74) | (SEQ ID NO: 43) |
| SP1 | | | |
| R4799-20 | R4799 | (SEQ ID NO: 79) | (SEQ ID NO: 43) |
| SP1 | | | |

Effector proteins were incubated with the described RNAs to form RNPs at a final concentration of 50 nM and allowed to incubate for 20 minutes at room temperature (at 37° C.) followed by addition of appropriate target nucleic acid at a final concentration of 0.1 nM. All assays were run at 50° C.

Cleavage activity was detected by fluorescence signal produced upon cleavage of a fluorophore-quencher reporter (T12) (included in the assay at 200 nM) in a QS5 machine.

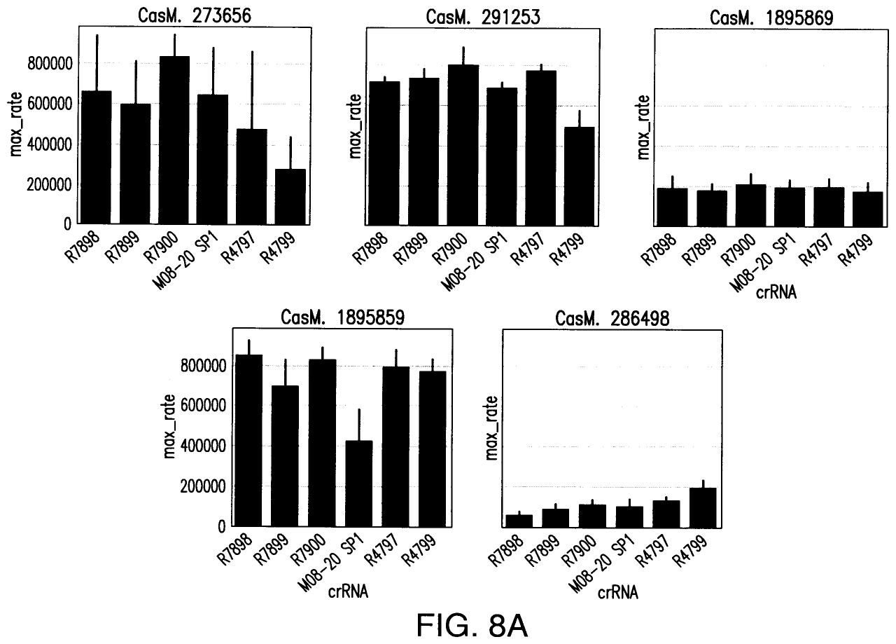

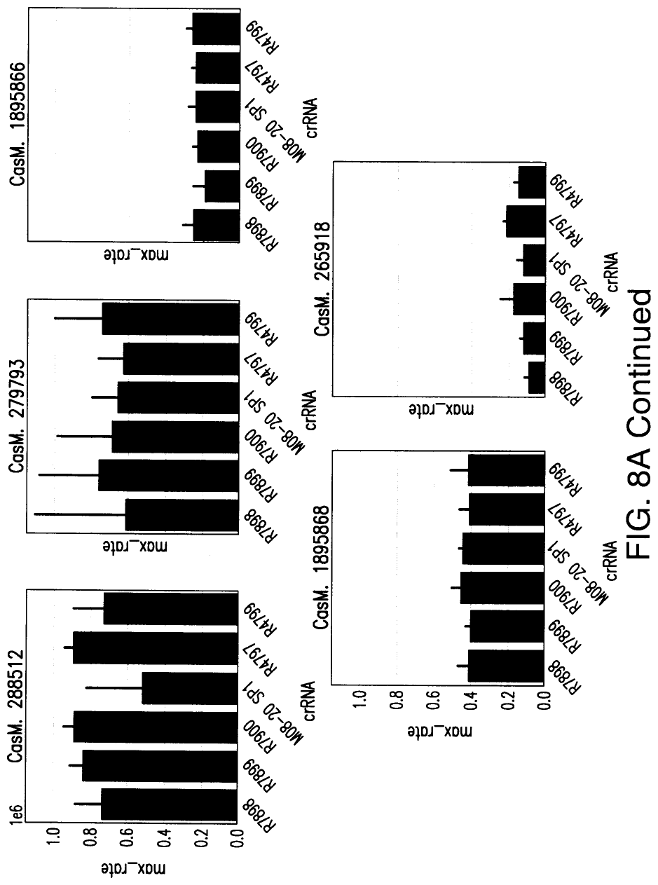

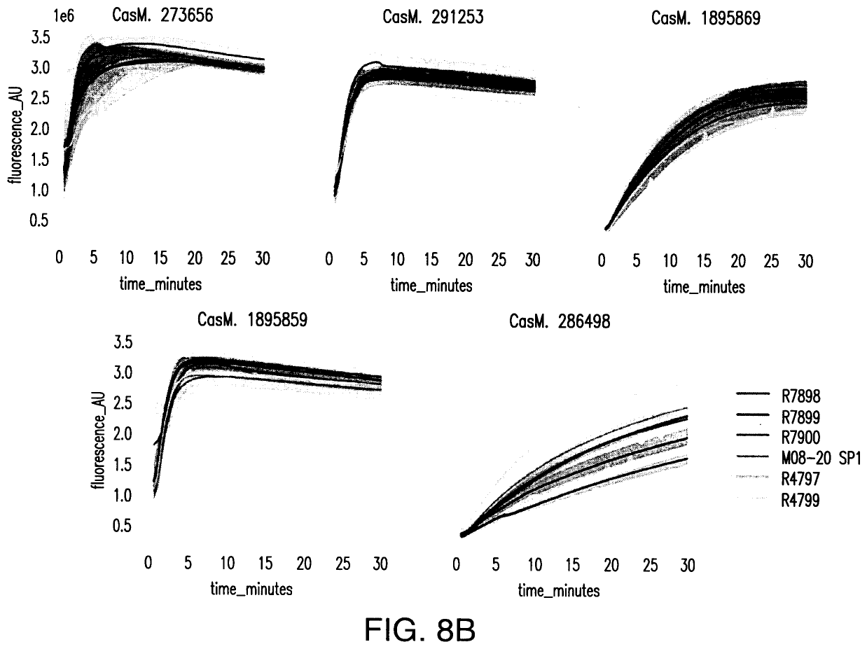

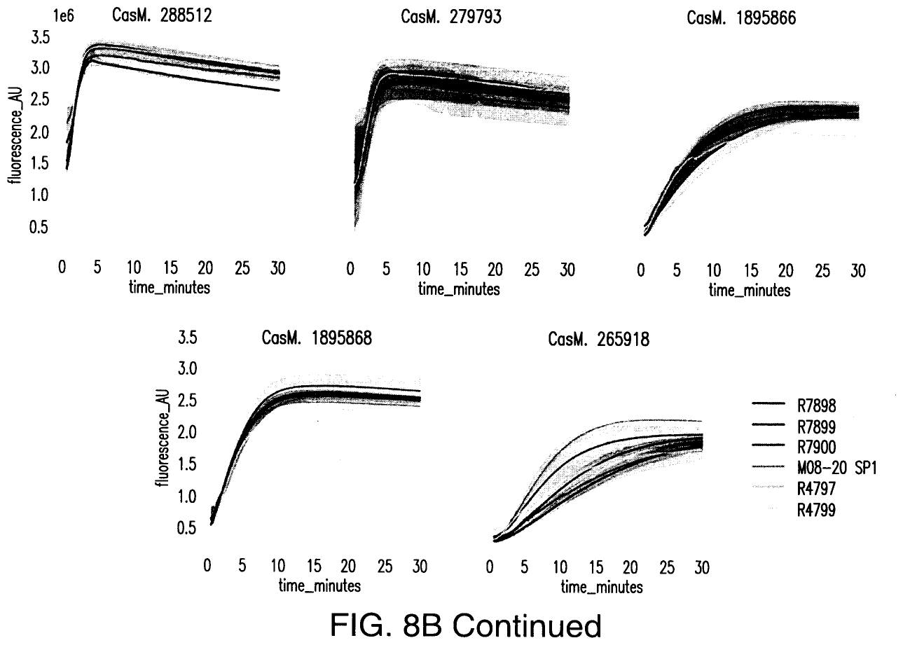

Results can be seen in FIG. 8A and FIG. 8B. The repeats tested supported roughly equal reaction profiles across all systems. The results in FIG. 8B show that all systems detected targets in less than 10 minutes. FIG. 8B also shows that PROTEIN ID NOS: 273656, 291253, 1895859, 288512, and 279793 reached maximal detection rates in less than about 5 minutes, whereas PROTEIN ID NO: 1895868 took between about 10 to 15 minutes and PROTEIN ID NOS: 1895869, 1895866, and 265918 took about 20 minutes or more to reach maximal detection rates.

### Example 11. Limit of Detection Assay

The following describes an experiment carried out to determine limit of detection (LOD) for effector proteins (PROTEIN ID NOS: 273656, 286498, 279793, 288512, 1895859, 291253, 265918, 1895869, 1895866, 21528) by determining the minimum target that each system is able to react with to produce meaningful data.

Effector proteins were incubated with RNAs to form RNPs under conditions similar to conditions in Examples 9 and 10. Effector Proteins were complexed with the same control RNA and targeted a 5′TTG S1 target (1.1 kb). Use of the control RNA allows the assay to utilize the same screening substrate between all RNP systems. The substrate was serially diluted to final concentrations ranging from 1 nM to 0.1 fM. Next, dilutions of 200 nM of fluorescent reporter (200 μM T12) and a different substrate were added to each RNP, and fluorescence was measured using a QS5 machine. A no-substrate, reporter only solution was also used as a control. All assays were run at 50° C. LOD was then determined by comparing the maximum rate of reaction and the general shape of the fluorescent readout time course to that of the no-substrate control.

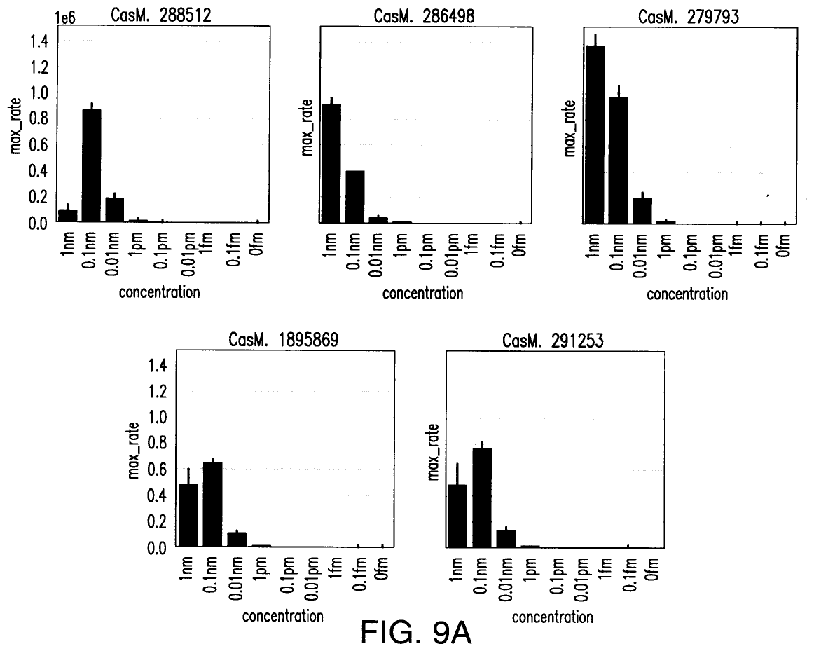

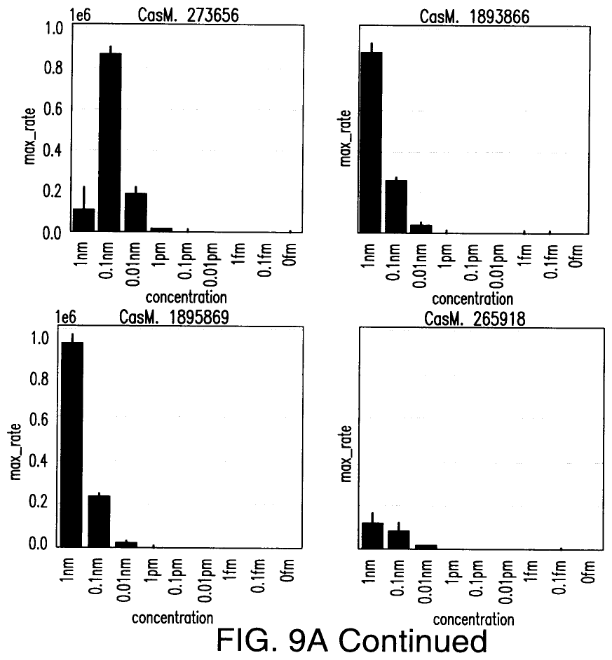

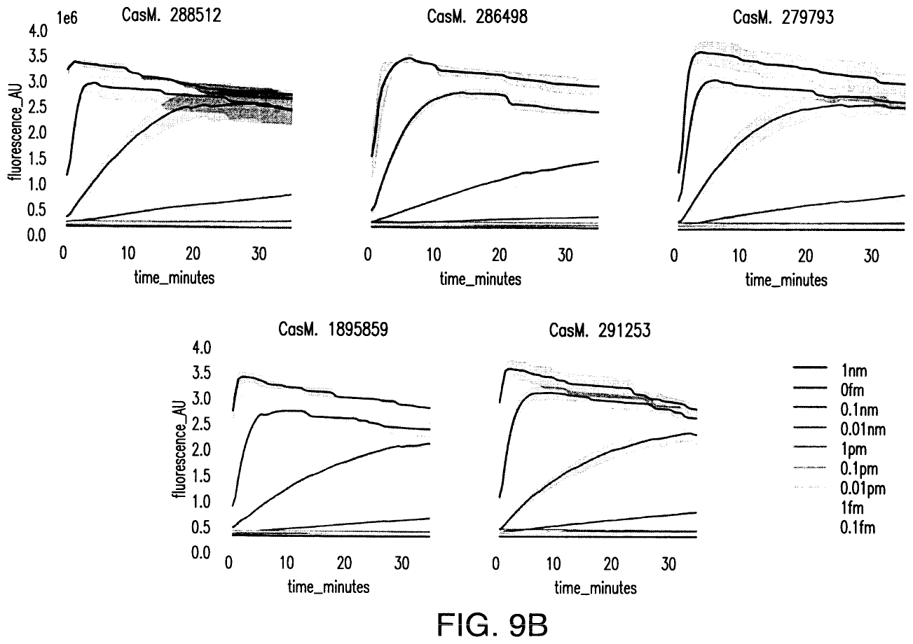

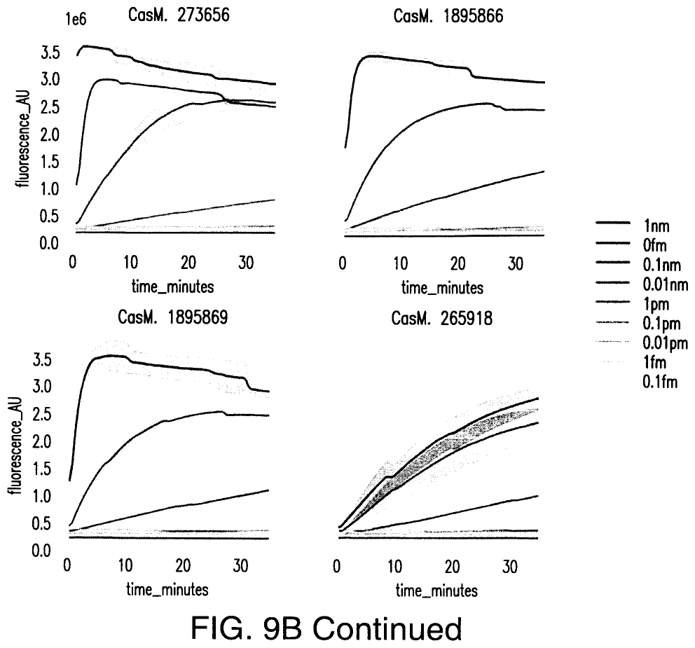

Results can be seen in TABLE 11 and FIGS. 9A and 9B. FIG. 9B shows that all exemplary enzymes tested detected target as low as 0.01 nm in less than 10 minutes. FIG. 9B also shows that PROTEIN ID NOS: 288512, 286498, 279793, 1895859, 291253, 273656, 1895866, and 1895869 reached maximal detection rates of the target at 1 nm in less than about 5 minutes; PROTEIN ID NOS: 288512, 279793, 291253, and 273656, reached maximal detection rates of the target at 0.1 nm in less than about 5 minutes; and PROTEIN ID NOS: 288512, 279793, 273656, at 0.01 nm in less than about 30 minutes.

TABLE 11
Limit of Detection

| Effector Protein SEQ ID NO: | PROTEIN ID NO: | LOD (pM) |
|---|---|---|
| 1 | 273656 | 1 |
| 2 | 286498 | 1 |
| 3 | 279793 | 1 |
| 4 | 288512 | 1 |
| 5 | 1895859 | 1 |
| 7 | 291253 | 1 |
| 8 | 265918 | 10 |
| 9 | 1895869 | 10 |
| 10 | 1895866 | 10 |
| 28 | 21528 | 1 |

### Example 12. Reporter Screen

This example describes experiments to determine the reporter preferences of various exemplary effector proteins (PROTEIN ID NOS: 273656, 286498, 279793, 288512, 1895859, 1895868, 291253, 265918, 1895869, and 1895866) and to identify reports that are suitable for multiplexing. Ten different reporters were tested (9 ssDNA and 1 ssRNA). The tested reporters are shown in TABLE 12.

TABLE 12
Reporter Descriptions

| | 5′ | | |
|---|---|---|---|
| Reporter | Detection | | 3′ |
| Name | Moiety* | Sequence (SEQ ID NO:) | Quencher* |
| 20 NT | /56-FAM/ | CAAGCTTCCCGTTCTCAGCC | /3BHQ_1/ |
| mixed | | (SEQ ID NO: 84) | |
| 10 TA- | /56-FAM/ | ATTATTATTA | /3IABkFQ/ |
| mixed | | (SEQ ID NO: 85) | |
| N12 | /FAM/ | NNNNNNNNNNNN, wherein | /IaBkFQ/ |
| | | each N is independently | |
| | | A, C, G or T | |
| | | (SEQ ID NO: 86) | |
| T12 | /56-FAM/ | TTTTTTTTTTTT | /3IABkFQ/ |
| | | (SEQ ID NO: 35) | |
| C12 | /56-FAM/ | CCCCCCCCCCCC | /3IABkFQ/ |
| | | (SEQ ID NO: 87) | |
| A12 | /56-FAM/ | AAAAAAAAAAAA | /3IABkFQ/ |
| | | (SEQ ID NO: 88) | |
| T5 | /56-FAM/ | TTTTT | /3IABkFQ/ |
| | | (SEQ ID NO: 89) | |
| 12N-RNA | /56-FAM/ | rNrNrNrNrNrNrNrNrNrNr | /3IABkFQ/ |
| | | NrN, wherein each rN is | |
| | | independently A, G, C, | |
| | | U or T | |
| | | (SEQ ID NO: 90) | |
| H12 | /56-FAM/ | HHHHHHHHHHHH, wherein | /3IABkFQ/ |
| | | each H is independently | |
| | | A, C, or T | |
| | | (SEQ ID NO: 91) | |
| T8 | /56-FAM/ | TTTTTTTT | /3IABkFQ/ |
| | | (SEQ ID NO: 92) | |

/56-FAM/: 5′ 6-Fluorescein (Integrated DNA Technologies)
/3IABkFQ/: 3′ Iowa Black FQ (Integrated DNA Technologies)
/3BHQ_1/: 3′ Black Hole Quencher®-1 Integrated DNA Technologies)
/IaBkFQ/: 3′ Iowa Black FQ (Integrated DNA Technologies)
/FAM/: fluorescein amidite
*TABLE 12 refers to the detection moiety and quencher moiety as their tradenames and their source is identified. However, alternatives, generics, or non-tradename moieties with similar function from other sources can also be used.

Effector proteins were incubated with guide RNAs at a 1:1 ratio to form RNPs at a final concentration of 50 nM under conditions similar to conditions in Examples 9-11. Guide RNAs were designed to target a 5′-TTTG-3′ PAM. The RNPs were combined with the appropriate DNA substrate (0.1 nM) and 200 nM fluorescent reporter and then placed into a qRT-PCR (QS5) machine for 30 minutes at 50C. Fluorescent readout was then used to evaluate the impact of each RNA and spacer length on enzyme performance.

Systems have widely differential ability to utilize short reporters (e.g., T5). Differential activity on T5 was first observed in DESPEC 3 and 5 systems demonstrated notable T5 signal.

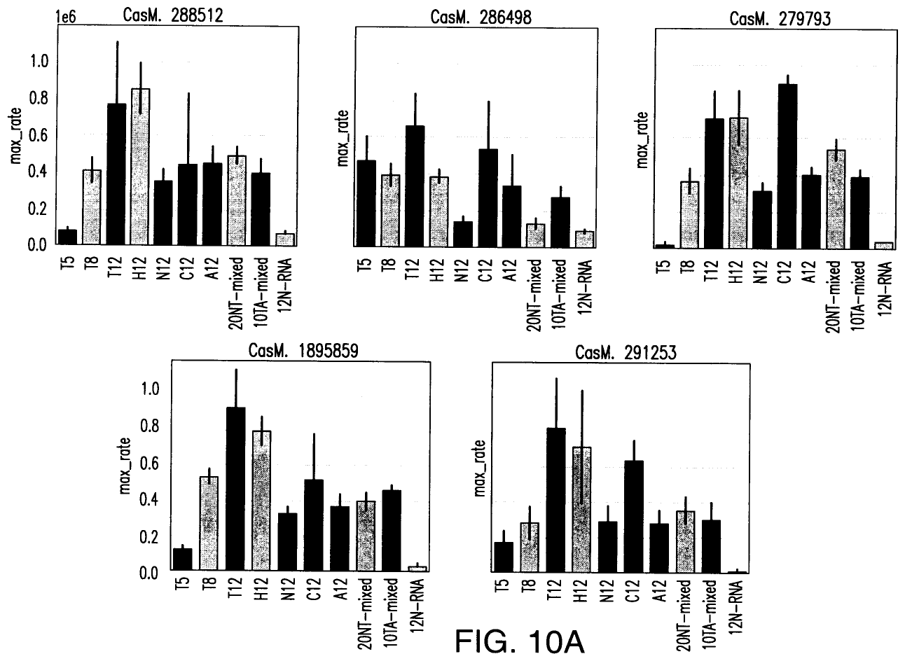

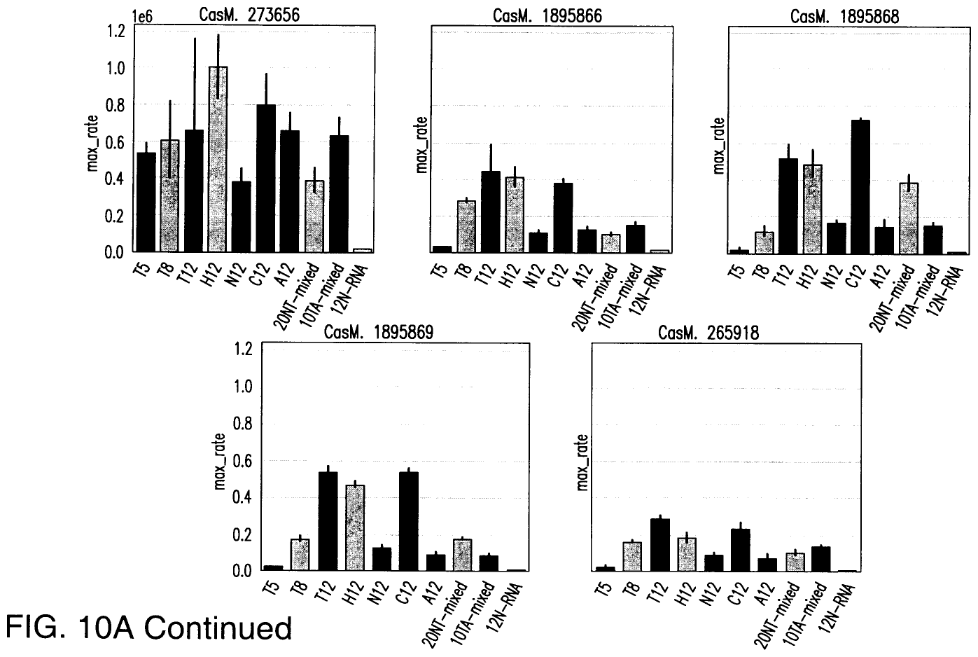

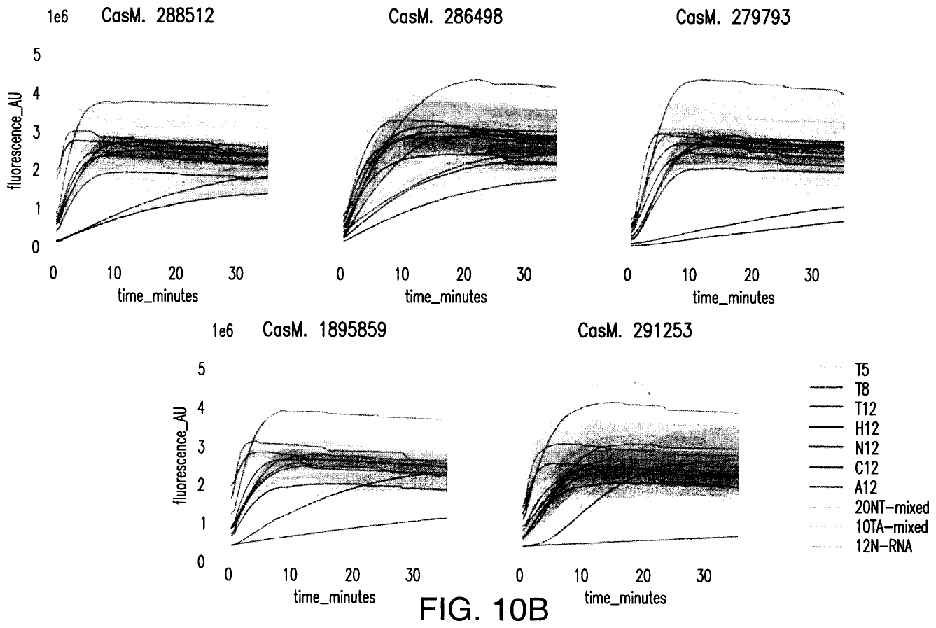

Results can be seen in FIGS. 10A and 10B. Generally, T-rich and H-rich (not G) reporters of length 12 nucleotides were most preferred between all systems, with one system showing a preference for C-rich reporters of length 12. FIG. 10B shows that all systems detected cleavage of the assayed reporters within less than 10 minutes, whereas maximal cleavage took between 5 and 30 minutes for most reporters, with reporters 12N-RNA and T5 being the least detectable reporters for most systems. FIG. 10B also shows that PROTEIN ID NOS: 288512, 1895859, 273656, 1895868 can reach maximal T12 and H12 digestion rate in less than 10 minutes.

Also, T12 or H12 (no Gs) reporters performed better than N12, suggesting secondary structure influences in the latter and identifying H12 as a particularly suitable screening reporter.

### Example 13. Thermostability and Salt Resistance

TABLE 13 shows the optimum temperature range and salt tolerance of effector proteins as determined from the assays described in the above examples.

TABLE 13
Thermostability and Salt Resistance of Effector Proteins

| PROTEIN | SEQ | Optimal | 100 mM Salt |
|---|---|---|---|
| ID NO: | ID NO: | Temp (C.) | Tolerance |
| 273656 | 1 | 50-55 | ++ |
| 286498 | 2 | 45 | ++ |
| 279793 | 3 | 50-60 | ++ |
| 288512 | 4 | 40-50 | +++ |
| 1895859 | 5 | 55-60 | +++ |
| 1895868 | 6 | 55-60 | ++ |
| 291253 | 7 | 60 | +++ |
| 265918 | 8 | 45-50 | ++ |
| 1895869 | 9 | 50-55 | ++ |
| 1895866 | 10 | ~40 | +++ |
| 21528 | 28 | 40-60 | ++ |

### Example 14. Two-Pot Detection with Effector Proteins at High Temperature

The following assay was carried out to assess compatibility of the effector proteins (PROTEIN ID NOs: 1895868 (SEQ ID NO: 6), 279793 (SEQ ID NO: 3), 189869 (SEQ ID NO: 5) with amplification product from a reverse transcription loop-mediated isothermal amplification (RT-LAMP)-based amplification assay.

To generate the amplification product, 2 μL of Influenza B (IVB) target RNA (for a final concentration of 100 copies (100 cp) per reaction or 0 copies (0 cp) per reaction) was combined with and 8 μL RT-LAMP mix of the following components (listed at final concentration): IB1 LAMP trans-cleavage buffer, dNTPs (1 mM), SYTO9 (50 μM), RNase Inhibitor, Bst 2.0 DNA polymerase, Warmstart RTx reverse transcriptase, and IVB LAMP primer mix. IB1 Buffer comprised: 20mMTris HCl pH8.8, 50 mM KoAc, 10 mM NH4SO4, 0.1% Tween 20, 5 mM MgOAc. IVB LAMP primers are as set forth in TABLE 14. The target RNA was then amplified using RT-LAMP for 60 minutes at 55° C.

TABLE 14
LAMP Primers

| | | SEQ |
|---|---|---|
| | | ID |
| Sequence | | NO: |
| TGCTAAACTTGTTGCTACTG | (F3) | 165 |
| TTCTTCCGTGACCAGTCT | (B3) | 166 |
| CGCTCGAAGAGTGAATTGAGGATGATCTT | (FIP) | 167 |
| ACAGTGGAGGATG | | |
| CTGCGGTGGGAGTCTTATCCGTCTCCCTC | (BIP) | 168 |
| TTCTGGTGAT | | |
| ATCCGATGGCCATCTTCTT | (LF) | 169 |
| CAATTTGGTCAAGAGCACCG | (LB) | 170 |

To determine the compatibility of the amplification product with the effector proteins (PROTEIN ID NOs: 1895868 (SEQ ID NO: 6), 279793 (SEQ ID NO: 3), 189869 (SEQ ID NO: 5), the amplification product of RT-LAMP was titrated into a trans-cleavage reaction to assess where the break point was, if any, and to determine whether the product can inhibit a trans-cleavage reaction. Briefly, 1895868 (SEQ ID NO: 6), 279793 (SEQ ID NO: 3), or 189869 (SEQ ID NO:5) effector proteins were complexed with crRNA for 30 minutes at 37° C. to form RNPs. Effector protein and crRNA combinations are set forth in TABLE 15, where bold indicates the repeat sequence and underline indicates the spacer sequence.

TABLE 15
ASSAYED EFFECTOR PROTEIN AND CRRNA COMBINATIONS

| Effector | Repeat | | crRNA |
|---|---|---|---|
| Protein | sequence | | SEQ ID |
| SEQ ID NO. | SEQ ID NO: | crRNA | NO: |
| 6 | 78 | UAAUUUCUACUAAGUGUAGAU | 163 |
| | | AGCUGCUCGAAUUGGCUUUG | |
| 3 and 5 | 79 | GAAUUUCUACUAUUGUAGAUA | 164 |
| | | AGCCAAUUCGAGCAGCUGA | |

The 1× concentration of effector protein was 40 nM and the final concentration of crRNAs was 40 nM. 1 μL of these RNPs was combined with a 16 μL mix of the following components for a total volume of ˜18 μL (listed at final concentration): IB1 LAMP trans-cleavage buffer, FQ reporter (1000 nM), TIPP (0.2 U), and varying volumes of RT-LAMP amplification product (1 μL to 8 μL). The FQ reporter used was the C12 reporter/5Alex594N/CCCCCCCCCCCC/3IAbRQSp/(SEQ ID NO: 87). Reactions were carried out at 58° C. for 60 minutes.

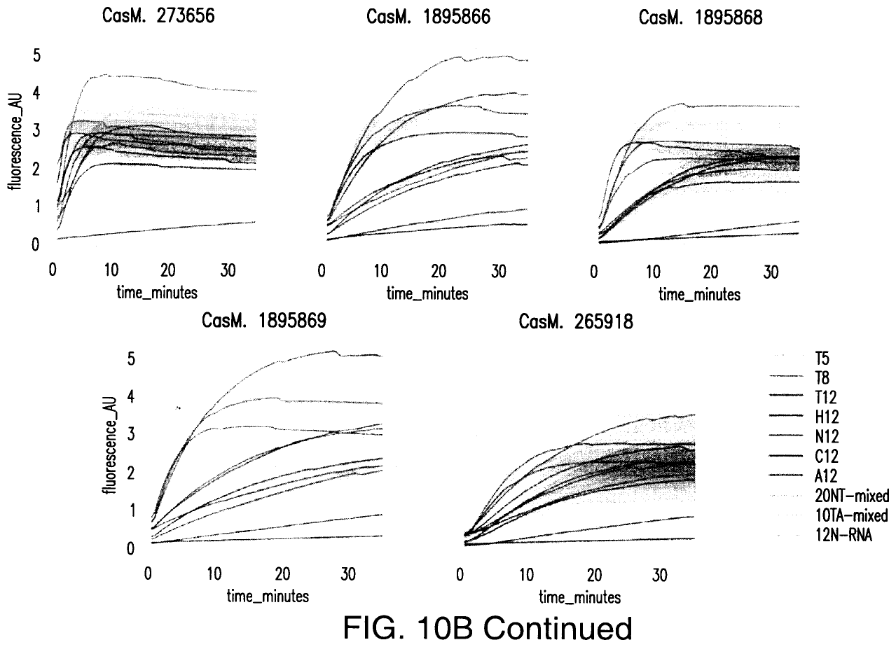

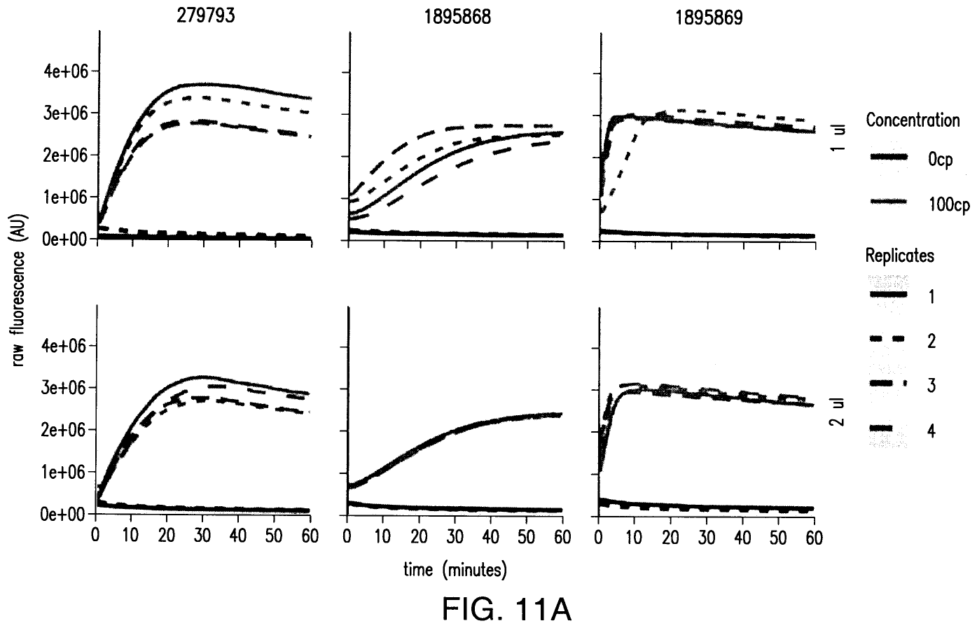

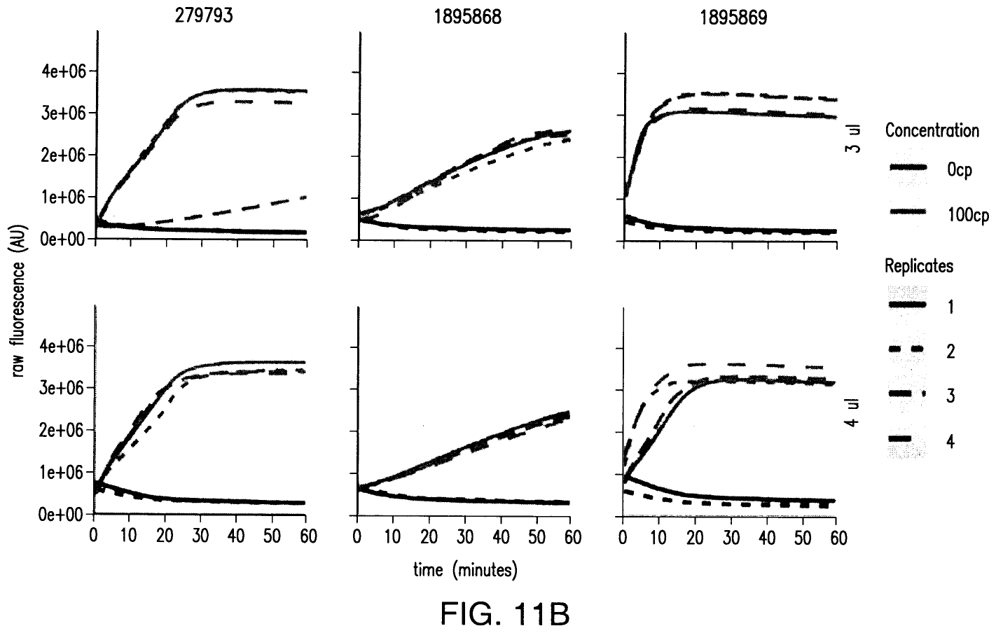

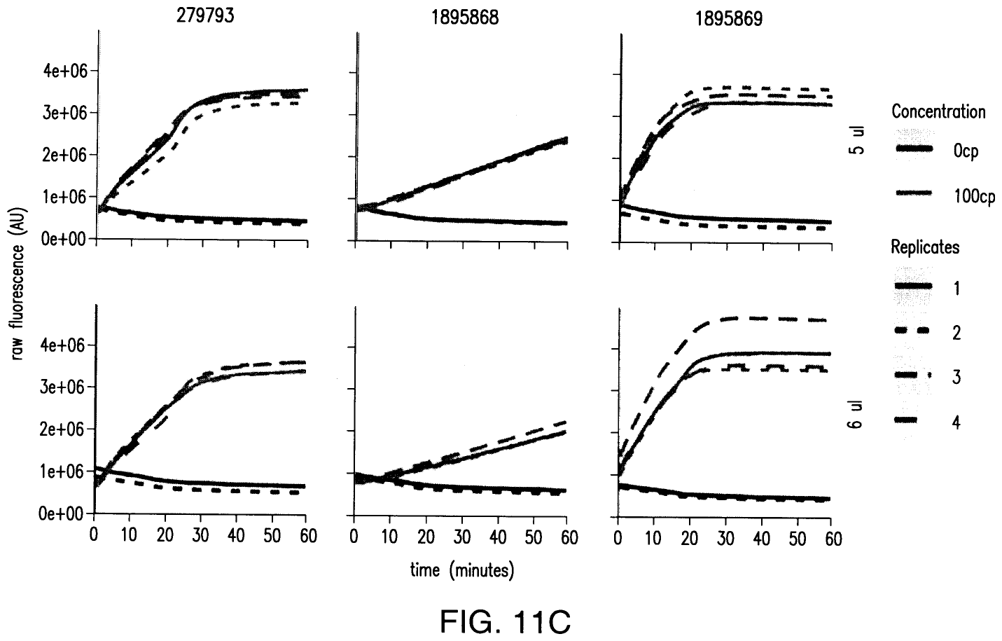

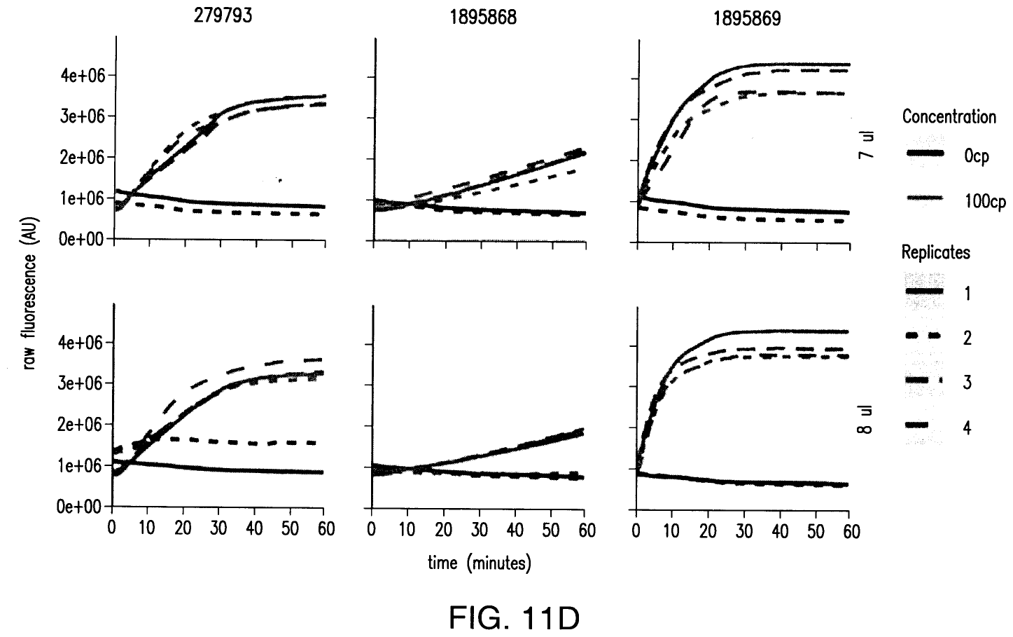

Trans-cleavage activity was detected by fluorescence signal upon cleavage of a fluorophore-quencher reporter in the trans-cleavage reaction. FIGS. 11A-11D shows performance of effector proteins 1895868 (SEQ ID NO: 6), 279793 (SEQ ID NO: 3), and 189869 (SEQ ID NO: 5) in the trans-cleavage reaction run at 58° C. with varying amounts of RT-LAMP amplification product added as the substrate.

All three effector proteins generated robust signals under the conditions tested, with some being more tolerant than others of higher concentrations of amplification product, indicating that the effectors proteins are compatible with assays utilizing RT-LAMP.

### Example 15: One-Pot Detection with Effector Proteins at High Temperature

The assay was carried out to assess compatibility of the effector proteins (PROTEIN ID NO: 1895868 (SEQ ID NO: 6)) with a one-pot detection method comprising RT-LAMP and DETECTR at high temperatures. Briefly, 1895868 (SEQ ID NO: 6) effector proteins were complexed with crRNAs, each comprising a repeat sequence of SEQ ID NO: 78 and targeting RSVB, IVB, SC2, or H3N2 for 30 minutes at 37° C. The 1× concentration of proteins was 40 nM and the final concentration of crRNAs was 320 nM. 1 μL of these RNPs was combined with a 9 μL mix of the following components for a total volume of ˜10 μL (listed at final concentration): IB1 LAMP trans-cleavage buffer, dNTPs (1 mM), SYTO9 (1 μM), RNase Inhibitor, Bst 2.0 DNA polymerase, glycerol-free Warmstart RTx reverse transcriptase, LAMP primer mix (RSVB, IVB, SARS-CoV-2 (SC2), or H3N2 Influenza A (IVA)), TIPP, target RNA (50 copies 50 (cp) per reaction or 0 copies (0 cp) per reaction of RSVB, IVB, SC2, or H3N2 substrate), and a C12 FQ reporter. The FQ reporter used was the C12 reporter/5Alex594N/CCCCCCCCCCCC/3IAbRQSp/(SEQ ID NO: 87). Reactions were carried out at 53° C. for 60 minutes. RT-LAMP activity was monitored by SYTO9 fluorescence signal generated during amplification and trans-cleavage activity was monitored by fluorescence signal upon cleavage of a fluorophore-quencher reporter by the activated RNP.

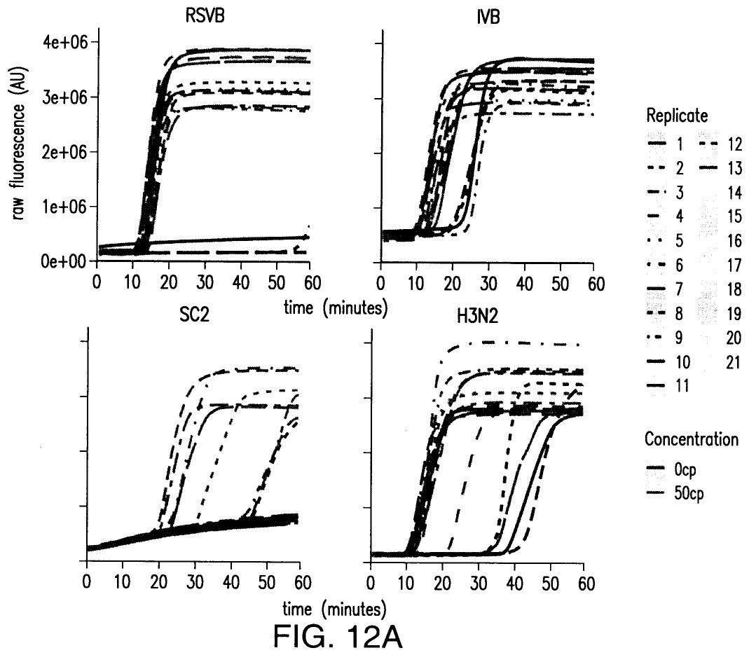

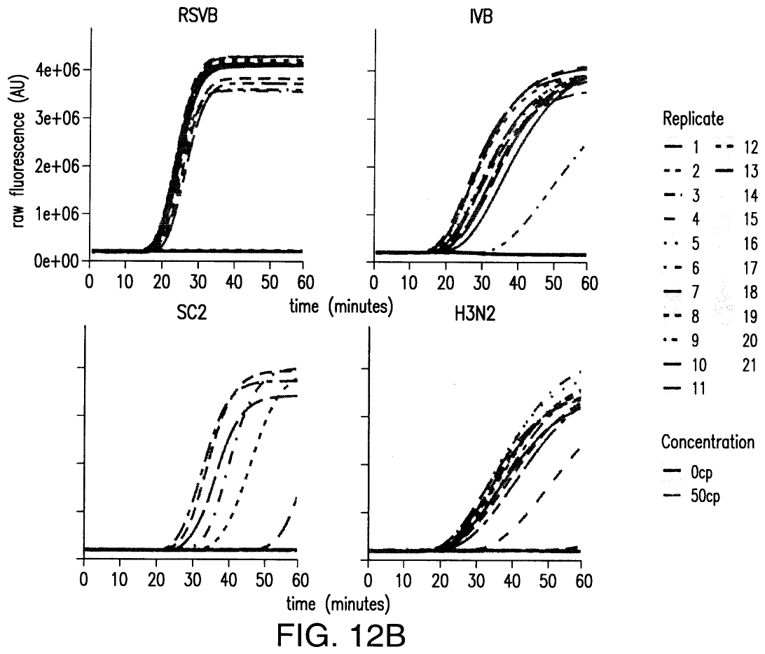

FIG. 12A shows RT-LAMP product generation in the one-pot RT-LAMP trans-cleavage reaction run at 53° C. for various target substrates. FIG. 12B shows performance of effector protein 1895868 (SEQ ID NO: 6) in the one-pot RT-LAMP trans-cleavage reaction run at 53° C. for various target substrates. For each target substrate, the effector protein was able to detect the target and generate sufficient fluorescence signal so as to distinguish between target with 50 copies per reaction (50 cp/rxn) and no target control with 0 copies per reaction (0 cp/rxn) within about 20 minutes or less under the conditions tested, even when non-specific RT-LAMP product was generated (as in some of the 0 copies per reaction negative control conditions for the IVB and H3N2 target substrates).

### Example 16: One-Pot Detection Sensitivity Assay

The following assay was carried out to determine the detection sensitivity for effector proteins (PROTEIN ID NO: 1895868 (SEQ ID NO: 6)) in a HotPot (RT-LAMP+DETECTR) assay under certain conditions by determining the minimum target that each system is able to react with to produce meaningful data under the conditions tested.

Briefly, 1895868 (SEQ ID NO: 6) effector proteins were complexed with crRNA comprising a repeat sequence of SEQ ID NO: 78 and targeting RSVB or RNaseP for 30 minutes at 37° C. The 1× concentration of proteins was 40 nM and the final concentration of crRNAs was 320 nM. 1 μL of these RNPs was combined with a 9 μL mix of the following components for a total volume of ˜10 μL (listed at final concentration): IB1 LAMP trans-cleavage buffer, dNTPs (1 mM), SYTO9 (1 μM), RNase Inhibitor, Bst 2.0 DNA polymerase, glycerol-free Warmstart RTx reverse transcriptase, LAMP primer mix (RSVB or RNaseP), TIPP, RSVB or RNaseP target RNA (varying concentrations), and a C12 FQ reporter. The FQ reporter used was the C12 reporter/5Alex594N/CCCCCCCCCCCC/3IAbRQSp/(SEQ ID NO: 87). The RSVB target RNA or RNaseP target RNA was serially diluted to final concentrations ranging from 100 copies (100 cp) per reaction to 0 copies (0 cp) per reaction. Reactions were carried out at 53° C. for 60 minutes. RT-LAMP activity was monitored by SYTO9 fluorescence signal generated during amplification and trans-cleavage activity was monitored by fluorescence signal upon cleavage of a fluorophore-quencher reporter by the activated RNP. The detection sensitivity of the effector proteins under the assay conditions was then determined by comparing the maximum rate of reaction and the general shape of the fluorescent readout time course to that of the no-substrate control (0 copies (0 cp)).

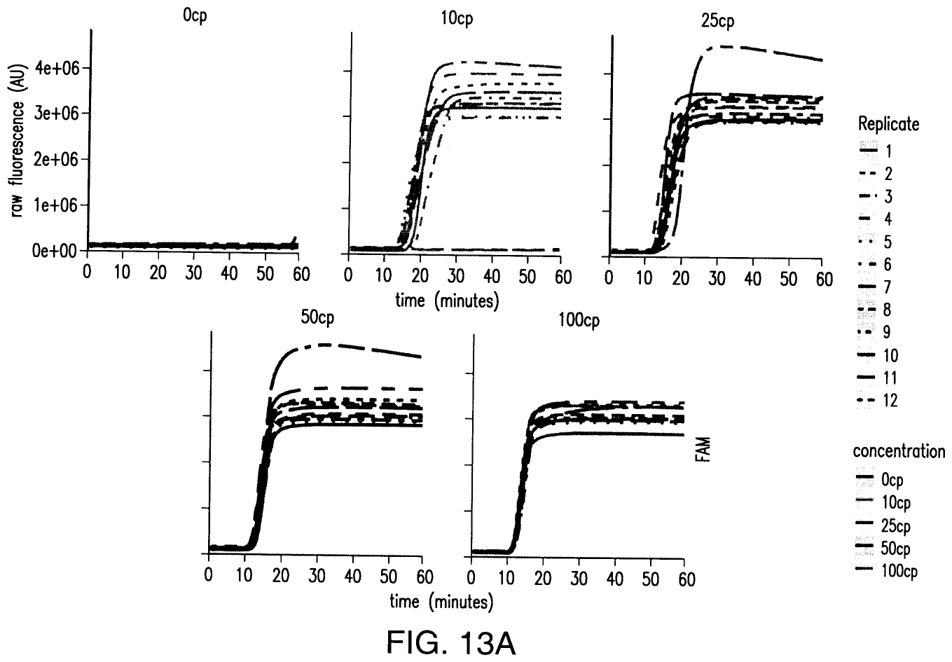

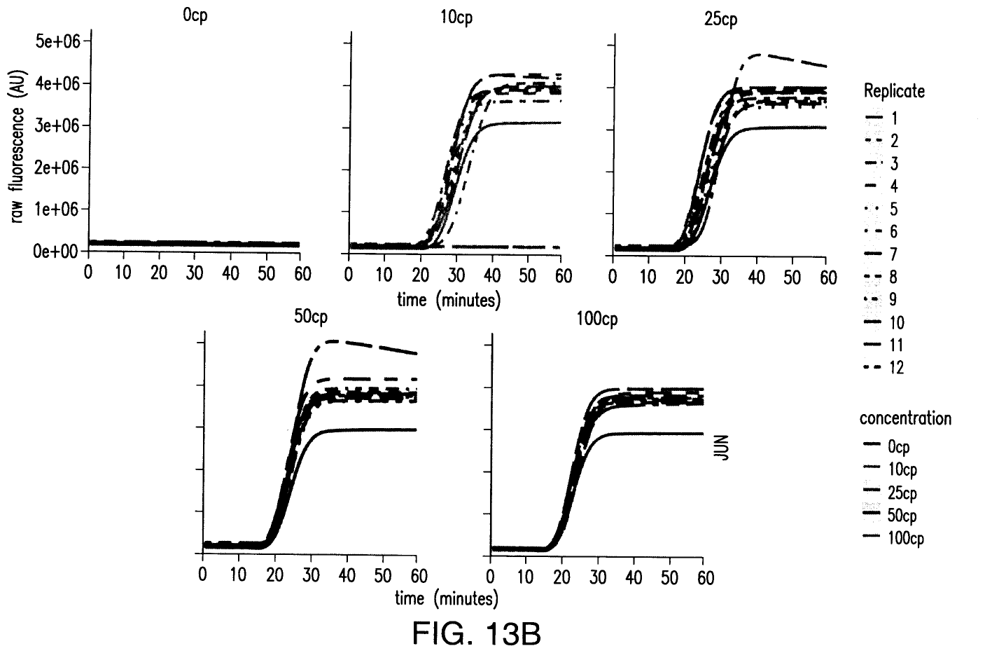

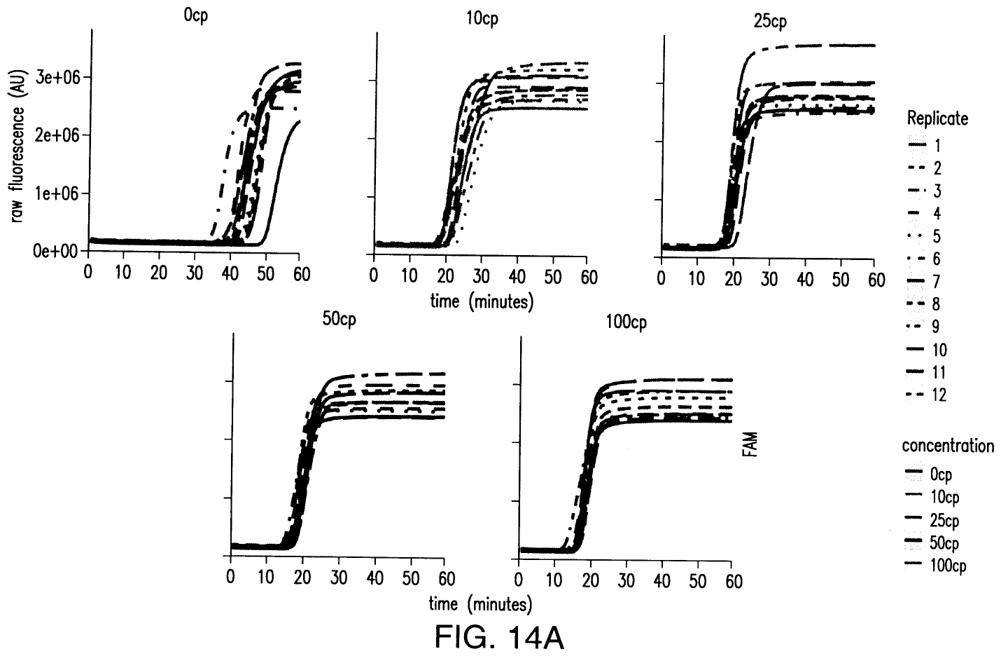

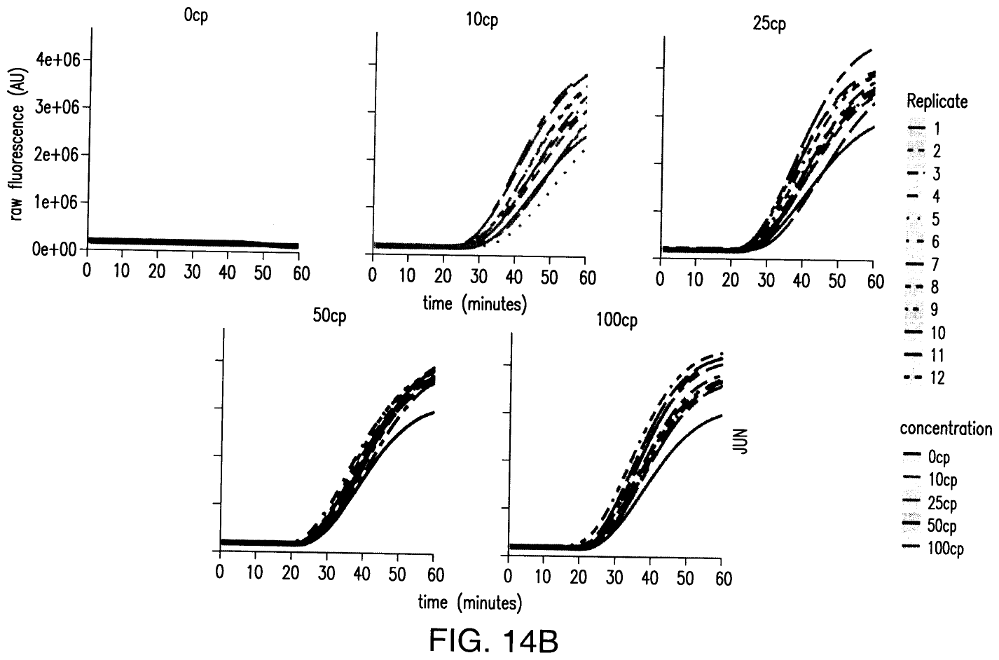

FIGS. 13A and 14A show RT-LAMP product generation in the one-pot RT-LAMP trans-cleavage reaction run at 53° C. for RSVB and RNaseP target substrates, respectively. FIGS. 13B and 14B show performance of effector protein 1895868 (SEQ ID NO: 6) in the one-pot RT-LAMP trans-cleavage reaction run at 53° C. for RSVB and RNaseP target substrates, respectively. The effector protein was able to detect all replicates of the RNaseP target, indicating a detection sensitivity of the effector proteins under the assay conditions of at least 10 copies (10 cp) per reaction, while the RSVB target was successfully detected in all but one replicate (FIG. 13A, second graph from the left), indicating a detection sensitivity of about 10 copies (10 cp) per reaction under the conditions tested. Importantly, this detection was target sequence specific, as exemplified in the case of the negative control RNaseP target condition where non-specific RT-LAMP amplification product was generated but no reporter fluorescence was observed.

### Example 17: One-Pot DETECTR Compatibility with Crude Sample Prep

The following assay was carried out to assess compatibility and detection sensitivity of the effector proteins (PROTEIN ID NO: 1895868 (SEQ ID NO: 6)) and crRNA comprising a repeat sequence of SEQ ID NO: 78 and targeting RNaseP or RSVB with low pH sample preparation followed by a HotPot (RT-LAMP+DETECTR) assay by determining the minimum target that each system is able to react with to produce meaningful data under the conditions tested.

Briefly, a crude nasal fluid sample comprising varying starting concentrations of added RSVB target RNA substrate (300 copies (300 cp), 150 copies (150 cp), 75 copies (75 cp), or 0 copies (0 cp) per reaction) was added to a low pH lysis buffer (comprising KOAc pH 4) and incubated for 5 minutes at 95° C. A neutralization buffer (comprising Tris base and KOH) was then added to stop the lysis reaction and raise the pH of the reaction mixture to pH 8.8 to be compatible with the trans-cleavage detection assay. The crude sample was lysed and then combined with the following components for a total volume of ˜15 μL (listed at final concentration): NH₄SO₄ (10 mM), Tween 20 (0.1%), MgOAc (5 mM), 1895868 effector protein (SEQ ID NO: 6) (40 nM), RSVB or RNaseP crRNA (320 nM), dNTPs (1 mM), SYTO9 (1 μM), RNase Inhibitor, Bst 2.0 DNA polymerase, glycerol-free Warmstart RTx reverse transcriptase, LAMP primer mix (RSVB or RNaseP), TIPP, RSVB or RNaseP target RNA (varying concentrations), and a C12 FQ reporter. The FQ reporter used was the C12 reporter/5Alex594N/CCCCCCCCCCCC/3IAbRQSp/(SEQ ID NO: 87).

Reactions were carried out at 53° C. for 60 minutes. RNaseP was used as a positive control for extraction and was expected to be present in any sample where RNA was successfully extracted from the nasal matrix. A negative control reaction was performed without nasal matrix (shown as 0 copies (0 cp) per reaction in the RNaseP column). RT-LAMP activity was monitored by SYTO9 fluorescence signal generated during amplification and trans-cleavage activity was monitored by fluorescence signal upon cleavage of a fluorophore-quencher reporter by the activated RNP. The detection sensitivity of the effector proteins under the assay conditions was then determined by comparing the maximum rate of reaction and the general shape of the fluorescent readout time course to that of the no-substrate control (0 copies (0 cp)).

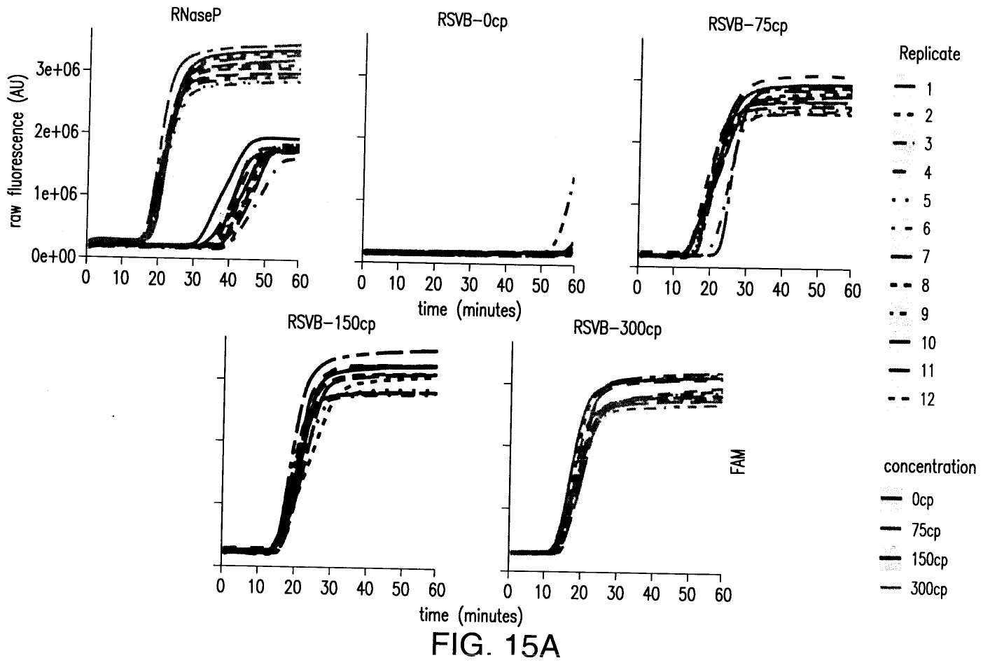

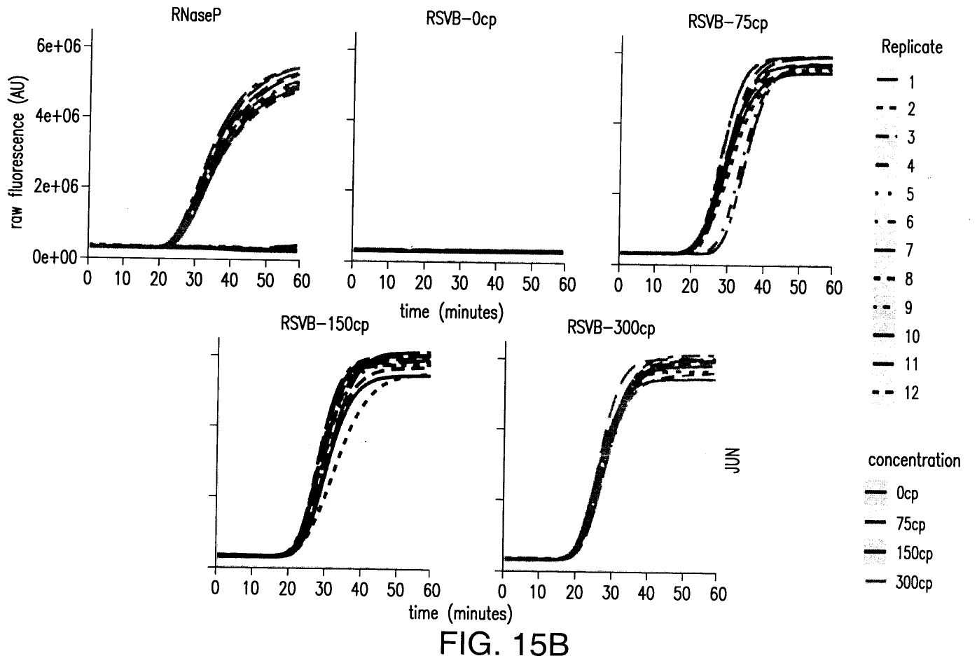

FIG. 15A shows RT-LAMP product generation in the one-pot RT-LAMP trans-cleavage reaction run at 53° C. for RSVB or RNaseP target substrates. FIG. 15B shows performance of effector protein 1895868 (SEQ ID NO: 6) in the one-pot RT-LAMP trans-cleavage reaction run at 53° C. for RSVB or RNaseP target substrates. The effector protein was able to successfully detect the RNaseP present in the nasal matrix, indicating successful extraction of RNA during sample preparation. The effector protein was also able to detect all replicates having RSVB spiked in, indicating a detection sensitivity of the effector proteins under the assay conditions of at least 75 copies (75 cp) per reaction under the conditions tested.

### Example 18. PAM Screening for Effector Proteins

Effector proteins and guide RNA combinations described herein are screened by an in vitro enrichment (IVE) assay to determine PAM recognition by each effector protein-guide RNA complex. Briefly, effector proteins are complexed with corresponding guide RNAs for 15 minutes at 37° C. The complexes are added to an IVE reaction mix. PAM screening reactions use 10 μl of RNP in 100 μl reactions with 1,000 ng of a 5′ PAM library in 1× Cutsmart buffer and are carried out for 15 minutes at 25° C., 45 minutes at 37° C., and 15 minutes at 45° C. Reactions are terminated with 1 μl of proteinase K and 5 μl of 500 mM EDTA for 30 minutes at 37° C. Next generation sequencing is performed on cut sequences to identify enriched PAMs.

### Example 19. Detecting Target Nucleic Acids

Effector proteins are tested for trans cleavage. Briefly, partially purified (e.g., nickel-NTA purified) effector proteins are incubated with crRNA and tracrRNA (or an sgRNA) in a trans cleavage buffer (e.g., 20 mM Tricine, 15 mM MgCl2, 0.2 mg/ml BSA, 1 mM TCEP (pH 9 at 37° C.)) at room temperature for about 10 to about 30 minutes, followed by addition of a target nucleic acid to produce effector-protein guide complexes. trans cleavage activity is detected by fluorescence signal upon cleavage of a fluorophore-quencher reporter. Dilutions of the effector-protein guide complexes are performed, and the assay repeated at various concentrations of the effector-protein guide complexes.

### Example 20. Effector Proteins Edit Genomic DNA in Mammalian Cells

Effector proteins are tested for their ability to produce indels in a mammalian cell line (e.g., HEK293T cells). Briefly, a plasmid encoding the effector proteins and a guide RNA are delivered by lipofection to the mammalian cells. This is performed with a variety of guide RNAs targeting several loci adjacent to biochemically determined PAM sequences. Indels in the loci are detected by next generation sequencing of PCR amplicons at the targeted loci and indel percentage is calculated as the fraction of sequencing reads containing insertions or deletions relative to an unedited reference sequence. “No plasmid” and Cas9 are included as negative and positive controls, respectively.

What is claimed is:

1. A system comprising:
(a) a polypeptide, or a nucleic acid encoding the polypeptide, wherein the polypeptide comprises an amino acid sequence that is at least 75% identical to any one of SEQ ID NOS: 7, 6, 3, 5, 1, 2, 4, 8-28 or 93-142; and
(b) an engineered guide nucleic acid, or a nucleic acid that encodes the engineered guide nucleic acid, wherein the engineered guide nucleic acid comprises a first region and a second region, wherein the first region comprises a nucleic acid sequence that is complementary to the target sequence in the target nucleic acid, wherein the first region and the second region are heterologous to each other.

2.-11. (canceled)

12. The system of claim 1, wherein engineered guide nucleic comprises a repeat sequence, wherein the repeat sequence comprises a nucleotide sequence that is at least 75% identical to any one of SEQ ID NOS: 74-79.

13.-28. (canceled)

29. The system of claim 1, wherein the target sequence is located adjacent to a protospacer adjacent motif (PAM) sequence in a target nucleic acid, wherein the polypeptide recognizes the PAM, and wherein the PAM comprises any one of NNN, NNNN, NNNNN, NNNNNN, NNNNNNN, YTTN, TTYN, TTTN, TTCN, or CTTN, wherein each N is independently any one of A, G, C, or T, and wherein Y is C or T.

30.-39. (canceled)

40. The system of claim 1, wherein the system further comprises an additional guide nucleic acid that binds a different loci of the target nucleic acid than the guide nucleic acid.

41.-45. (canceled)

46. The system of claim 1, wherein the polypeptide comprises an activity in a solution comprising salt, wherein the concentration of a salt in the solution is from about 0.001 mM to 200 mM.

47. The system of claim 1, wherein the polypeptide comprises an activity in a solution, wherein a temperature of the solution is from about 37° C. to about 65° C.

48.-54. (canceled)

55. The system of claim 1, wherein the polypeptide comprises an enhanced activity compared to a Cas12 protein.

56. The system of claim 1, comprising a salt in a solution comprising the polypeptide.

57. The system of claim 56, wherein the salt is potassium acetate, sodium chloride, or ammonium sulfate.

58.-59. (canceled)

60. The system of claim 1, comprising a solution comprising the polypeptide wherein the solution is from about 37° C. to about 65° C.

61. (canceled)

62. The system of claim 1, wherein the system further comprises one or more of:
(a) a detection reagent; and/or
(b) an amplification reagent.

63.-65. (canceled)

66. A system for detecting a target nucleic acid, comprising the system of claim 62, wherein the detection reagent comprises a reporter comprising a reporter nucleic acid and a detection moiety.

67. The system of claim 66, wherein cleavage of the reporter generates a detectable product or detectable signal from the detectable moiety.

68.-72. (canceled)

73. The system of claim 66, wherein the polypeptide is effective to cleave the reporter in response to formation of a complex comprising the polypeptide, the engineered guide nucleic acid, and the target nucleic acid.

74.-85. (canceled)

86. A method of detecting a presence of a target nucleic acid in a sample, comprising the steps of:
(a) contacting the sample with:
(i) the system of claim 1; and
(b) cleaving a reporter with the polypeptide in response to formation of a complex comprising the polypeptide, an engineered guide nucleic acid, and a target sequence in a target nucleic acid, thereby producing a detectable product; and
(c) detecting the detectable product, thereby detecting the presence of the target nucleic acid in the sample.

87.-111. (canceled)

112. A method of modifying a target nucleic acid, the method comprising contacting the target nucleic acid with the system of claim 1, thereby producing a modified target nucleic acid.

113.-141. (canceled)

142. A system comprising:
(a) a polypeptide, or a nucleic acid encoding the polypeptide, and an engineered guide nucleic acid, or a nucleic acid that encodes the engineered guide nucleic acid;
(b) a polypeptide, or a nucleic acid encoding the polypeptide, and an engineered guide nucleic acid comprising a single guide nucleic acid;
(c) a polypeptide, or a nucleic acid encoding the polypeptide, an engineered guide nucleic acid, and a donor nucleic acid;
(d) a polypeptide, or a nucleic acid encoding the polypeptide, an engineered guide nucleic acid comprising a single guide nucleic acid, and a donor nucleic acid;
(e) an mRNA encoding a polypeptide, and an engineered guide nucleic acid;
(f) an mRNA encoding a polypeptide, an engineered guide RNA, and a donor nucleic acid;
(g) one or more recombinant expression vectors comprising: i) a nucleic acid encoding a polypeptide; and ii) an engineered guide nucleic acid;
(h) one or more recombinant expression vectors comprising: i) a nucleic acid encoding a polypeptide; ii) an engineered guide nucleic acid; and iii) and a donor nucleic acid;
(i) one or more recombinant expression vectors comprising: i) a nucleic acid encoding a polypeptide; and ii) an engineered guide nucleic acid comprising a single guide nucleic acid; or
(j) one or more recombinant expression vectors comprising: i) a nucleic acid encoding a polypeptide; ii) an engineered guide nucleic acid comprising a single guide nucleic acid; and iii) and a donor nucleic acid;
wherein the polypeptide comprises an amino acid sequence that is at least 85% identical to any one of SEQ ID NOS: 7, 6, 3, 5, 1, 2, 4, 8-28 or 93-142.

143.-147. (canceled)

148. A device comprising:
(a) a polypeptide, or a nucleic acid encoding the polypeptide, and an engineered guide nucleic acid, or a nucleic acid that encodes the engineered guide nucleic acid;
(b) a polypeptide, or a nucleic acid encoding the polypeptide, and an engineered guide nucleic acid comprising a single guide nucleic acid;
(c) a polypeptide, or a nucleic acid encoding the polypeptide, an engineered guide nucleic acid, and a donor nucleic acid;
(d) a polypeptide, or a nucleic acid encoding the polypeptide, an engineered guide nucleic acid comprising a single guide nucleic acid, and a donor nucleic acid;
(e) an mRNA encoding a polypeptide, and an engineered guide nucleic acid;
(f) an mRNA encoding a polypeptide, an engineered guide RNA, and a donor nucleic acid;
(g) one or more recombinant expression vectors comprising: i) a nucleic acid encoding a polypeptide; and ii) an engineered guide nucleic acid;
(h) one or more recombinant expression vectors comprising: i) a nucleic acid encoding a polypeptide; ii) an engineered guide nucleic acid; and iii) and a donor nucleic acid;
(i) one or more recombinant expression vectors comprising: i) a nucleic acid encoding a polypeptide; and ii) an engineered guide nucleic acid comprising a single guide nucleic acid; or
(j) one or more recombinant expression vectors comprising: i) a nucleic acid encoding a polypeptide; ii) an engineered guide nucleic acid comprising a single guide nucleic acid; and iii) and a donor nucleic acid;
wherein the polypeptide comprises an amino acid sequence that is at least 85% identical to any one of SEQ ID NOS: 7, 6, 3, 5, 1, 2, 4, 8-28 or 93-142.

149.-150. (canceled)

151. A microfluidic device comprising:
(a) a sample interface configured to receive a sample comprising nucleic acids;
(b) a chamber fluidically connected to the sample interface;
wherein the chamber comprises a polypeptide and an engineered guide nucleic acid,
wherein the polypeptide comprises an amino acid sequence that is at least 85% identical to any one of SEQ ID NOS: 7, 6, 3, 5, 1, 2, 4, 8-28 or 93-142.

152.-169. (canceled)

170. A composition comprising:
(a) a polypeptide, or a nucleic acid encoding the polypeptide, and an engineered guide nucleic acid, or a nucleic acid that encodes the engineered guide nucleic acid;
(b) a polypeptide, or a nucleic acid encoding the polypeptide, and an engineered guide nucleic acid comprising a single guide nucleic acid;
(c) a polypeptide, or a nucleic acid encoding the polypeptide, an engineered guide nucleic acid, and a donor nucleic acid;
(d) a polypeptide, or a nucleic acid encoding the polypeptide, an engineered guide nucleic acid comprising a single guide nucleic acid, and a donor nucleic acid;
(e) an mRNA encoding a polypeptide, and an engineered guide nucleic acid;
(f) an mRNA encoding a polypeptide, an engineered guide RNA, and a donor nucleic acid;
(g) one or more recombinant expression vectors comprising: i) a nucleic acid encoding a polypeptide; and ii) an engineered guide nucleic acid;
(h) one or more recombinant expression vectors comprising: i) a nucleic acid encoding a polypeptide; ii) an engineered guide nucleic acid; and iii) and a donor nucleic acid;
(i) one or more recombinant expression vectors comprising: i) a nucleic acid encoding a polypeptide; and ii) an engineered guide nucleic acid comprising a single guide nucleic acid; or
(j) one or more recombinant expression vectors comprising: i) a nucleic acid encoding a polypeptide; ii) an engineered guide nucleic acid comprising a single guide nucleic acid; and iii) and a donor nucleic acid;
wherein the polypeptide comprises an amino acid sequence that is at least 85% identical to any one of SEQ ID NOS: 7, 6, 3, 5, 1, 2, 4, 8-28 or 93-142.

171.-180. (canceled)
